(19) United States
(12) Patent Application Publication
(10) Pub. No.: US 2025/0161494 A1
(43) Pub. Date: May 22, 2025

(54) TRANSMEMBRANE RECEPTOR GENE EDITING

(71) Applicant: ORTHOBIO THERAPEUTICS, INC., Vail, CO (US)

(72) Inventors: Peter J. Millet, Edwards, CO (US); Iain Alasdair Russell, Cambridge (GB); Matthew J. Allen, Swaffham Bulbeck (GB); George Gentsch, Cambridge (GB)

(21) Appl. No.: 18/729,791

(22) PCT Filed: Jan. 19, 2023
(86) PCT No.: PCT/US2023/060939

§ 371 Date: Jul. 17, 2024

Related U.S. Application Data
(60) Provisional application No. 63/301,033, filed on Jan. 19, 2022.
(60) Provisional application No. 63/390,222, filed on Jul. 18, 2022.

(51) Int. Cl. A61K 48/00; C12N 9/22; C12N 15/11; C12N 15/86

(52) U.S. Cl. CPC A61K 48/0058; C12N 9/22; C12N 15/11; C12N 15/86; C12N 2310/20; C12N 2750/14143

(57) ABSTRACT

Provided herein are compositions and methods for ablating intracellular signaling through specific cell surface receptors as means of treatment for various conditions of a pro-inflammatory character. In some aspects, the compositions and methods are to prevent the progression of osteoarthritis and other arthritides and to treat osteoarthritis and other arthritides in a mammalian joint. In some aspects, the compositions and method are for treating or preventing localized nociception, inflammation, degeneration, or morphological changes associated with back or spine conditions or disorders.

### CROSS REFERENCE TO RELATED APPLICATIONS

This application claims priority to U.S. Provisional Patent Application No. 63/301,033, filed Jan. 19, 2022, U.S. Provisional Patent Application No. 63/303,479, filed Jan. 26, 2022, and U.S. Provisional Patent Application No. 63/390,222, filed Jul. 18, 2022, the contents of which are hereby incorporated by reference herein, in their entireties, for all purposes.

### BACKGROUND OF THE INVENTION

Receptor-ligand interactions are responsible for transmission of various signals across the plasma membrane of a receptor-expressing cell. In most instances, a circulating ligand will bind to a particular receptor anchored into cellular membrane (with or without co-receptors), which, by different mechanisms and often through activity of the receptor's cytoplasmic domain, results in the transduction of signaling pathways from the cell surface to the interior. Such signaling events can, in turn, impact a variety of cellular activities, including modulation of expression of various gene products that can further impact cellular functions.

The ubiquity of the receptor-ligand paradigm in cellular biology means that numerous diseases, illnesses, and conditions are caused, wholly or in part, by aberrant or excessive signaling through various cellular receptors, and various approaches have been utilized to address this. Small and large molecules can be used to disrupt receptor-ligand interactions, though issues of off-target effects and potential immunogenicity remain.

More recently, genetic approaches have been explored to either transiently reduce (i.e., knockdown), e.g., in the case of siRNA, or permanently ablate (i.e., genetic knockout), the expression of a given ligand or receptor. In both instances, such reduction or ablation can result in ‘leakiness.’ For example, a lower-affinity receptor may bind ligand present in excess in the absence of the native receptor, or conversely, unoccupied receptors may bind other ligands in absence of its native ligand. In either instance, this leakiness can result in additional issues at the organismal level. Therefore, more advanced genetic tools are needed to address the myriad of diseases, illnesses and conditions without the leakiness observed with traditional genetic knockdown or knockout.

### BRIEF SUMMARY OF THE INVENTION

Provided herein are compositions and methods for silencing the signaling functionality of one or more cellular receptors in an animal in need thereof to treat a disease, illness or condition caused by aberrant or excessive signaling through said receptor.

In some embodiments, receptor signaling is silenced by CRISPR editing of the gene encoding the receptor. In some embodiments, the CRISPR editing results in ablation of a transmembrane domain (e.g., generation of soluble decoy receptor). In some embodiments, the CRISPR editing results in ablation of a cytoplasmic domain (e.g., generation of a membrane-bound decoy receptor). In some embodiments, the CRISPR editing results targets the extracellular domain (e.g., generation of a complete knock out).

In contrast to complete ablation or transient knockdown of gene expression of a receptor or ligand, which has been associated with potentially important off-target effects and leakiness, the targeting of a receptor's transmembrane domain or cytoplasmic domain, as described herein, comprises a novel approach for blocking intracellular signaling of one or more receptor-ligand interactions without the issues of excess ligand or unoccupied receptors to contribute to more deleterious outcomes.

### BRIEF DESCRIPTION OF THE DRAWINGS

The presently disclosed embodiments will be further explained with reference to the attached drawings. The drawings shown are not necessarily to scale, with emphasis instead generally being placed upon illustrating the principles of the presently disclosed embodiments.

FIG. 1 illustrates the exons to be edited by CRISPR methods herein described to generate the indicated type of interference (e.g., genetic knockout, soluble decoy receptor, or membrane-bound decoy receptor) for the indicated exemplary gene targets.

FIGS. 2A and 2B illustrate the different types of CRISPR edits to (A) canine IL1RAP gene and (B) human TNFRSF4 gene. For cIL1RAP, the splicing map at top indicates which exons are edited with sgRNA shown, aligned with the corresponding domains in the full length cIL1RAP protein, such that OCP02 edits in the ectodomain, OCP07 edits in the transmembrane domain, and OCP10 edits in the TIR domain (bottom). For TNFRSF4, the splicing map at bottom is labeled with the encoded protein domains and aligned with the sgRNA binding sites and the interference type generated. For instance, those sgRNAs that generate soluble decoy receptors cluster within the area encoding the transmembrane domain, while those generating transmembrane decoy receptors cluster within the encoded cytoplasmic domain and ablate the TRAF binding sites. In either case, synthesis of the ectodomain is unimpacted, allowing binding of the ligand.

FIGS. 3A and 3B illustrate a non-exhaustive list of disease states associated with IL1R1 and IL1RAP activity.

FIGS. 4A and 4B illustrate a non-exhaustive list of disease states associated with (A) IL6R and (B) IL6ST activity.

FIG. 5 illustrates a non-exhaustive list of disease states associated with TNFRSF1A activity.

FIG. 6 illustrates a non-exhaustive list of disease states associated with TNFRSF1B activity.

FIG. 7 illustrates a non-exhaustive list of disease states associated with TNFRSF3 activity.

FIG. 8 illustrates a non-exhaustive list of disease states associated with TNFRSF4 activity.

FIG. 9 illustrates a non-exhaustive list of disease states associated with TNFRSF11A activity.

FIGS. 10A and 10B illustrate a non-exhaustive list of disease states associated with (A) TGFRB1 and (B) TGFBR2 activity.

FIGS. 11A and 11B illustrate the design of exemplary sgRNAs that target canine IL1R1, including (A) a summary of select sgRNAs based on off-target risks, on-target efficacy, and frameshift likelihood and (B) AlphaFold2 models of wild-type and decoy IL1R1 receptors, as predicted to be generated by OCR13 and OCR14.

FIG. 12 illustrates the in vitro performance of the tested sgRNA candidates that target canine IL1RAP, as deduced from Sanger traces. ND, not determined.

FIGS. 13A, 13B, 13C, and 13D illustrate the effect of various Cas9 variants on the in-vitro editing performance of select candidate sgRNAs that target canine IL1R1.

FIGS. 14A and 14B illustrate the design of exemplary sgRNAs that target canine IL1RAP, including (A) a summary of select sgRNAs based on off-target risks, on-target efficiency and frameshift likelihood and (B) AlphaFold2-predicted models of the 3D structure of normal and OCP07-edited IL1RAP.

FIG. 15 illustrates the in vitro performance of the tested sgRNA candidates that target canine IL1RAP, as deduced from Sanger traces.

FIGS. 16A, 16B, 16C illustrate (A) select sgRNAs targeting IL1RAP for testing and their editing efficacy with wildtype Cas9, (B) the effect of the indicated Cas9 variants on editing efficacy in canine monocytes and (C) a comparison of editing efficacy between AR-Cas9 and WT-Cas9 in canine synovial fibroblasts.

FIGS. 17A, 17B, and 17C illustrate the editing efficacy of the indicated IL1RAP-directed sgRNAs in (A) canine monocytes, (B) canine chondrocytes and (C) canine synovial fibroblasts.

FIG. 18 illustrates the impact of IL1R1 editing on silencing transcriptional induction of PTGS2 in response to 4 hours exposure to IL1β in canine synoviocytes.

FIG. 19 illustrates the impact of IL1R1 editing on silencing transcriptional induction of IL6 in response to 4 hours exposure to IL1β in canine synoviocytes.

FIG. 20 illustrates the experimental design underlying transcriptome analyses, among other assays. Generally, IL1β treatment will induce pro-inflammatory signaling exclusively through the IL1 receptor, whereas MSU crystals and LPS trigger inflammation by different means (in addition to inducing IL1 signaling).

FIGS. 21A and 21B illustrate the transcriptional upregulation of PTGS2 (COX-2) in wild type control and IL1RAP-edited canine monocytes at (A) 4 and (B) 24 hours of exposure to LPS (1 ug/ml), IL1β (100 pM), MSU crystals (400 μg/ml), and PBS (1×).

FIGS. 22A and 22B illustrate the impact of IL1RAP editing on silencing transcriptional induction of PTGS2 in (A) canine chondrocytes and (B) canine synoviocytes in response to 4 hours exposure to IL1β.

FIGS. 23A and 23B illustrate luciferase activity levels (RLU) of (A) canine synovial fibroblasts at 8 hours and (B) canine chondrocytes at 24 hours post exposure to luciferase mRNA containing LNPs at the indicated concentration.

FIGS. 24A and 24B illustrate results of synovial fluid cytology assays collected (A) at baseline and (B) at 4 weeks after injection of saline, low dose LNP formulation A and low-dose LNP formulation B.

FIGS. 25A, 25B, 25C, 25D, 25E, 25F, 25G, and 25H collectively illustrate, for control and IL1RAP-edited cells, (A) a principal component analysis plot of normalized gene counts following IL1β or MSU treatment. Two heatmaps of select gene expression as result of (B) IL1β or (C) MSU treatment, and Venn diagrams of upregulated or downregulated genes following (D, E) IL1β or (F, G) MSU treatment. (H) Transcriptome analysis of the top 500 IL1B-responsive genes in IL1RAP-edited canine monocytes DH82.

FIGS. 26A, 26B, 26C, 26D, 26E, and 26F collectively illustrate SEQ ID NOs: 680-824 (A-D) the crRNA sequences generated by the bioinformatic methods herein described that target human IL1R1 to generate a genetic knockout, a soluble decoy receptor, a membrane-bound decoy receptor, or other form and (E-H) additional information regarding the chromosome 2 genomic coordinates (assembly hg38) of the bound DNA, DNA strand targeted, exon targeted, and several predicted performance metrics.

FIGS. 27A, 27B, 27C, 27D, 27E, 27F, 27G, and 27H collectively illustrate SEQ ID NOs: 825-892 and SEQ ID NOs: 3336-3420 (A-D) the crRNA sequences generated by the bioinformatic methods herein described that target canine IL1R1 to generate a genetic knockout, a soluble decoy receptor, or a membrane-bound decoy receptor, or other form and (E-H) additional information includes the chromosome 10 genomic coordinates (assembly canFam3) of the bound DNA, the DNA strand targeted, the exon targeted, and several predicted performance metrics.

FIGS. 28A, 28B, and 28C collectively illustrate SEQ ID NOs: 893-967, the crRNA sequences generated by the bioinformatic methods herein described that target equine IL1R1 to generate (A) a genetic knockout, (B) a soluble decoy receptor, or (C) a membrane-bound decoy receptor. Additional information includes the chromosome 15 genomic coordinates (assembly equCab3) of the bound DNA, the DNA strand targeted, the exon targeted, and several predicted performance metrics.

FIGS. 29A, 29B, and 29C collectively illustrate SEQ ID NOs: 968-1039, the crRNA sequences generated by the bioinformatic methods herein described that target feline IL1R1 to generate (A) a genetic knockout, (B) a soluble decoy receptor, or (C) a membrane-bound decoy receptor. Additional information includes the chromosome A3 genomic coordinates (assembly felCat9) of the bound DNA, the DNA strand targeted, the exon targeted, and several predicted performance metrics.

FIGS. 30A, 30B, 30C, 30D, 30E, 30F, 30G, and 30H collectively illustrate SEQ ID NOs: 1040-1203, (A-D) the crRNA sequences generated by the bioinformatic methods herein described that target human IL1RAP to generate a genetic knockout, a soluble decoy receptor, a membrane-bound decoy receptor or other form and (E-H) additional information regarding the chromosome 3 genomic coordinates (assembly hg38) of the bound DNA, DNA strand targeted, exon targeted, and several predicted performance metrics.

FIGS. 31A, 31B, 31C, 31D, 31E, and 31F collectively illustrate SEQ ID NOs: 1204-1271 and SEQ ID NOs: 3421-3490 (A-C) the crRNA sequences generated by the bioinformatic methods herein described that target canine IL1RAP to generate a genetic knockout, a soluble decoy receptor, a membrane-bound decoy receptor, or other form and (D-F) additional information includes the chromosome 34 genomic coordinates (assembly canFam3) of the bound DNA, the DNA strand targeted, the exon targeted, and several predicted performance metrics.

FIGS. 32A, 32B, and 32C collectively illustrate SEQ ID NOs: 1272-1348, the crRNA sequences generated by the bioinformatic methods herein described that target equine IL1RAP to generate (A) a genetic knockout, (B) a soluble decoy receptor, or (C) a membrane-bound decoy receptor. Additional information includes the chromosome 19 genomic coordinates (assembly equCab3) of the bound DNA, the DNA strand targeted, the exon targeted, and several predicted performance metrics.

FIGS. 33A, 33B, and 33C collectively illustrate SEQ ID NOs: 1349-1424, the crRNA sequences generated by the bioinformatic methods herein described that target feline IL1RAP to generate (A) a genetic knockout, (B) a soluble decoy receptor, or (C) a membrane-bound decoy receptor. Additional information includes where the chromosome C2 genomic coordinates (assembly felCat9) bound, DNA strand targeted, exon targeted, and several predicted performance metrics.

FIGS. 34A, 34B, 34C, 34D, 34E, and 34F collectively illustrate SEQ ID NOs: 1425-1546, (A-C) the crRNA sequences generated by the bioinformatic methods herein described that target human TGFBR1 to generate a genetic knockout, a soluble decoy receptor, a membrane-bound decoy receptor or other form and (D-F) additional information regarding the chromosome 9 genomic coordinates (assembly hg38) of the bound DNA, DNA strand targeted, exon targeted, and several predicted performance metrics.

FIGS. 35A, 35B, 35C, 35D, 35E, 35F, 35G, and 35H collectively illustrate SEQ ID NOs: 1547-1745, (A-E) the crRNA sequences generated by the bioinformatic methods herein described that target human TGFBR2 to generate a genetic knockout, a soluble decoy receptor, a membrane-bound decoy receptor or other form and (F-J) additional information regarding the chromosome 3 genomic coordinates (assembly hg38) of the bound DNA, DNA strand targeted, exon targeted, and several predicted performance metrics.

FIGS. 36A, 36B, 36C, 36D, 36E, 36F, 36G, 36H, 36I, and 36J collectively illustrate SEQ ID NOs: 1746-1968, (A-E) the crRNA sequences generated by the bioinformatic methods herein described that target human IL6R to generate a genetic knockout, a soluble decoy receptor, a membrane-bound decoy receptor or other form and (F-J) additional information regarding the chromosome 1 genomic coordinates (assembly hg38) of the bound DNA, DNA strand targeted, exon targeted, and several predicted performance metrics.

FIGS. 37A, 37B, 37C, 37D, 37E, 37F, 37G, 37H, 37I, and 37J collectively illustrate SEQ ID NOs: 1969-2178, (A-E) the crRNA sequences generated by the bioinformatic methods herein described that target human IL6ST to generate a genetic knockout, a soluble decoy receptor, a membrane-bound decoy receptor or other form and (F-J) additional information regarding the chromosome 5 genomic coordinates (assembly hg38) of the bound DNA, DNA strand targeted, exon targeted, and several predicted performance metrics.

FIGS. 38A, 38B, 38C, 38D, 38E, 38F, 38G, 38H, 38I, and 38J collectively illustrate SEQ ID NOs: 2179-2395, (A-E) the crRNA sequences generated by the bioinformatic methods herein described that target human TNFRSF1A to generate a genetic knockout, a soluble decoy receptor, a membrane-bound decoy receptor or other form (F-J) additional information regarding the chromosome 12 genomic coordinates (assembly hg38) of the bound DNA, DNA strand targeted, exon targeted, and several predicted performance metrics.

FIGS. 39A, 39B, 39C, 39D, 39E, 39F, 39G, 39H, 39I, and 39J collectively illustrate SEQ ID NOs: 2396-2642, (A-E) the crRNA sequences generated by the bioinformatic methods herein described that target human TNFRSF1B to generate a genetic knockout, a soluble decoy receptor, a membrane-bound decoy receptor or other form and (F-J) additional information regarding the chromosome 1 genomic coordinates (assembly hg38) of the bound DNA, DNA strand targeted, exon targeted, and several predicted performance metrics.

FIGS. 40A, 40B, 40C, 40D, 40E, 40F, 40G, 40H, 40I, and 40J collectively illustrate SEQ ID NOs: 2643-2866, (A-E) the crRNA sequences generated by the bioinformatic methods herein described that target human TNFRSF3 to generate a genetic knockout, a soluble decoy receptor, a membrane-bound decoy receptor or other form and (F-J) additional information regarding the chromosome 12 genomic coordinates (assembly hg38) of the bound DNA, DNA strand targeted, exon targeted, and several predicted performance metrics.

FIGS. 41A, 41B, 41C, 41D, 41E, 41F, 41G, and 41H collectively illustrate SEQ ID NOs: 2867-3041, (A-D) the crRNA sequences generated by the bioinformatic methods herein described that target human TNFRSF4 to generate a genetic knockout, a soluble decoy receptor, a membrane-bound decoy receptor or other form and (E-H) additional information regarding the chromosome 1 genomic coordinates (assembly hg38) of the bound DNA, DNA strand targeted, exon targeted, and several predicted performance metrics.

FIGS. 42A, 42B, 42C, 42D, 42E, 42F, 42G, 42H, 42I, 42J, 42K, and 42L collectively illustrate SEQ ID NOs: 3042-3335, (A-F) the crRNA sequences generated by the bioinformatic methods herein described that target human TNFRSF11A to generate a genetic knockout, a soluble decoy receptor, a membrane-bound decoy receptor or other form and (G-L) additional information regarding the chromosome 18 genomic coordinates (assembly hg38) of the bound DNA, DNA strand targeted, exon targeted, and several predicted performance metrics.

FIGS. 43A and 43B illustrate (A) a schematic view showing the targeted domains of TGFBR1 and the orientation of various sgRNAs predicted to generate knockouts, membrane-bound decoy receptors, and soluble decoy receptors (ECD: Extracellular Domain; TMD: Transmembrane Domain; ICD: Intracellular Domain; GSM: GS rich Motif); and (B) a set of parameters considered for designing sgRNAs against TGFBR1, as well as example crRNA sequences.

FIGS. 44A and 44B illustrate (A) a schematic view showing the targeted domains of TGFBR2 and the orientation of various sgRNAs predicted to generate knockouts, membrane-bound decoy receptors, and soluble decoy receptors (ECD: Extracellular Domain; TMD: Transmembrane Domain; ICD: Intracellular Domain; GSM: GS rich Motif); and (B) a set of parameters considered for designing sgRNAs against TGFBR2, as well as example crRNA sequences.

FIGS. 45A and 45B show a summary of the efficiency for editing the human TGFBR1 gene in THP-1 cells using the identified guides and (A) wild type SpCas9 or (B) ARCas9.

FIGS. 46A and 46B show a summary of the efficiency for editing the human TGFBR2 gene in THP-1 cells using the identified guides and (A) wild type SpCas9 or (B) ARCas9.

FIGS. 47A, 47B, 47C, and 47D illustrate relative levels of (A, C) TGFB1 and (B, D) TIMP1 gene expression in THP-1 cells that are unedited (WT) or edited to knock out TGFB1 (OHTG), TGFBR1 (OHTIR), or TGFBR2 (OHTIIR) following challenge by LPS (panels A and B) or TGF-beta (panels C and D) for 6 hours.

FIGS. 48A, 48B, 48C, 48D, 48E, 48F, 48G, 48H, 48I, 48J, and 48K collectively illustrate human-directed crRNA sequences targeting (A) hIL1R1 (SEQ ID NOs: 3491-3513), (B) hIL1RAP (SEQ ID NOs: 3514-3543), (C) hIL6R (SEQ ID NOs: 3544-3566), (D) hIL6ST (SEQ ID NOs: 3567-3606), (E) hTNFRSF1A (SEQ ID NOs: 3607-3647), (F) hTNFRSF1B (SEQ ID NOs: 3648-3692), (G) hTNFRSF3 (SEQ ID NOs: 3693-3713), (H) hTNFRSF4 (SEQ ID NOs: 3714-3740), (I) hTNFRSF11A (SEQ ID NOs: 3741-3788), (J) hTGFBR1 (SEQ ID NOs: 3789-3813), and (K) hTGFBR2 (SEQ ID NOs: 3814-3865) for use in sgRNAs to validate in vitro editing with different modes of delivery.

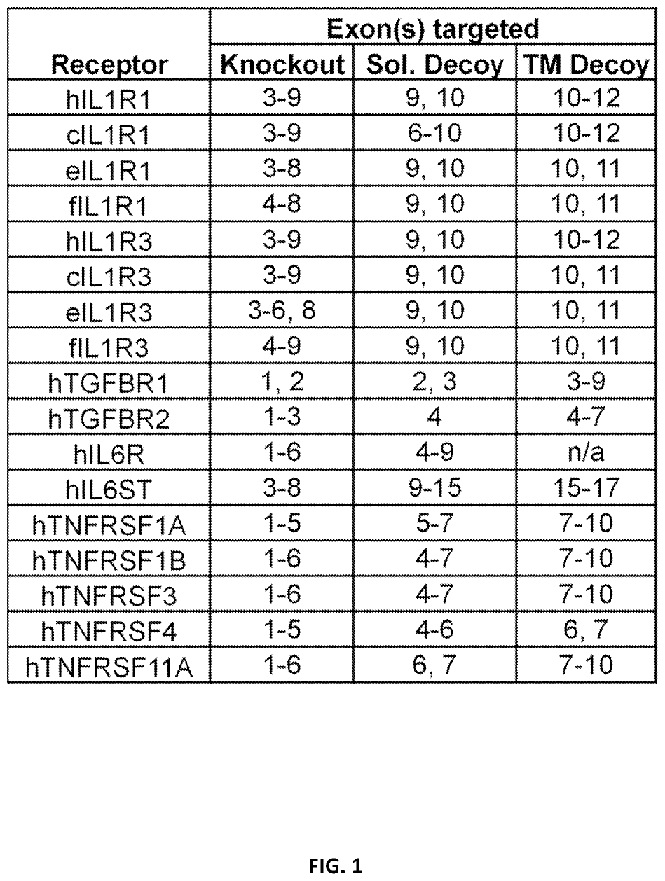

### DETAILED DESCRIPTION OF THE INVENTION

### I. Introduction

Provided herein are compositions and methods for silencing the signaling functionality of one or more cellular receptors in an animal in need thereof to treat a disease, illness or condition caused by aberrant or excessive signaling through said receptor.

In some embodiments, receptor signaling is silenced by CRISPR editing of the gene encoding the receptor. In some embodiments, the CRISPR editing results in ablation of a transmembrane domain (i.e., generation of a soluble decoy receptor). In some embodiments, the CRISPR editing results in ablation of a cytoplasmic domain (i.e., generation of a membrane-bound decoy receptor).

### II. Definitions

Unless defined otherwise, all technical and scientific terms used herein have the same meaning as is commonly understood by one of skill in the art to which this invention belongs. All patents and publications referred to herein are incorporated by reference in their entireties.

The terms “Interleukin 1 receptor type 1” or “IL1R1” refer to the genes (NCBI Gene ID: 3554 [human], NCBI Gene ID: 481328 [canine], NCBI Gene ID: 100009699 [equine], NCBI Gene ID: 101080705 [feline]) or an encoded gene product (e.g., UniProt: P14778; NP_001307909.1 [human], XP_038536135.1 [canine], NP_001075263.2 [equine], XP_023107327.2 [feline]), as well as sequence variants, proteins harboring conservative amino acid substitutions, and glycoforms thereof. Canonically, the proteins encoded by the genes listed above are capable of binding all forms of the pro-inflammatory cytokine interleukin 1 (IL1 or IL1) to mediate interleukin-1-dependent activation of NF-kappa-B, MAPK and other signaling pathways. This intracellular signaling involves the recruitment of adapter molecules such as TOLLIP, MYD88, and IRAK1 or IRAK2 via TIR-TIR interactions with the cytoplasmic domains of receptor/coreceptor subunits. IL1R1 can also bind the Interleukin 1 receptor antagonist (IL1Ra or IL1Ra or IL1RN), which prevents association with IL1RAP to form a signaling-competent complex. In some instances, and merely for the sake of disambiguation, a prefix is added when referring to the protein or gene of a particular species (with h, c, e, and f, referring to the human, canine, equine, and feline forms, respectively).

In certain embodiments, any region of an IL1R1 gene (e.g., 5′ untranslated region [UTR], exon 1, exon 2, exon 3, exon 4, exon 5, exon 6, exon 7, exon 8, exon 9, exon 10, exon 11, exon 12, exon 13, exon 14, exon 15, exon 16, exon 17, exon 18, exon 19, exon 20, exon 21, any intervening intronic regions, intron/exon junctions, the 3′ UTR, or polyadenylation signal) is targeted by an RNA-guided nuclease to alter the gene. In some embodiments, the IL1R1 gene targeted by an RNA-guided nuclease is from a mammal. In some embodiments, the IL1R1 gene targeted by an RNA-guided nuclease is from a human (hIL1R1). In some embodiments, the IL1R1 gene targeted by an RNA-guided nuclease is from a dog (cIL1R1). In some embodiments, the IL1R1 gene targeted by an RNA-guided nuclease is from a horse (eIL1R1). In some embodiments, the IL1R1 gene targeted by an RNA-guided nuclease is from a cat (fIL1R1).

The terms “Interleukin 1 Receptor Accessory Protein,” “IL1RAP,” or “IL1RAP” refer to the genes (NCBI Gene ID: 3556 [human], NCBI Gene ID: 488126 [canine], NCBI Gene ID: 100068726 [equine], NCBI Gene ID: 101094125 [feline]) or an encoded gene product (e.g., UniProt: Q9NPH3; NP_002173.1 [human], XP_038318680.1 [canine], XP_001498597.2 [equine], XP_044893081.1 [feline]), as well as sequence variants, proteins harboring conservative amino acid substitutions, and glycoforms thereof. Canonically, the proteins encoded by the genes listed above are capable of associating with IL1R1 bound to IL1 to form the high affinity interleukin-1 receptor complex that mediates interleukin-1-dependent activation of NF-kappa-B and other signaling pathways through the recruitment of adapter molecules such as TOLLIP, MYD88, and IRAK1 or IRAK2 via TIR-TIR interactions with the cytoplasmic domains of receptor/coreceptor subunits. In some instances, and merely for the sake of disambiguation, a prefix is added when referring to the protein or gene of a particular species (with h, c, e, and f, referring to the human, canine, equine, and feline forms, respectively).

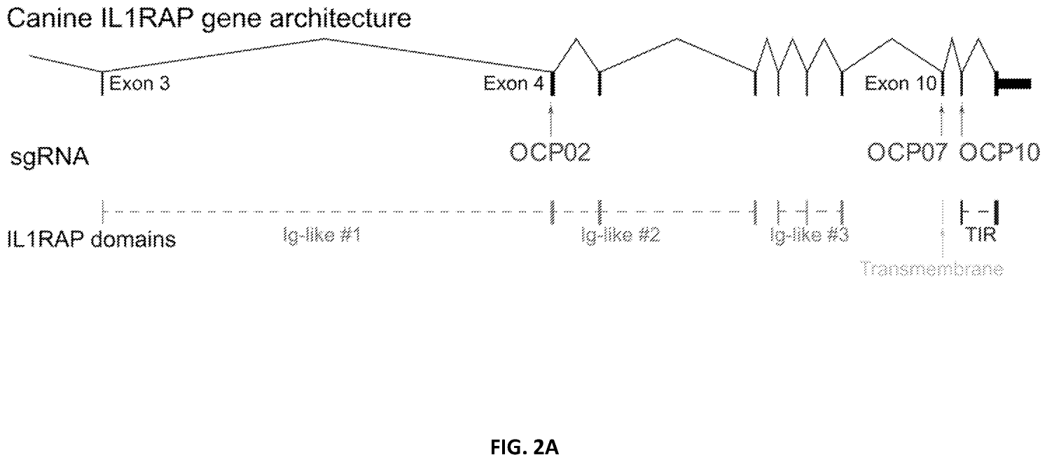

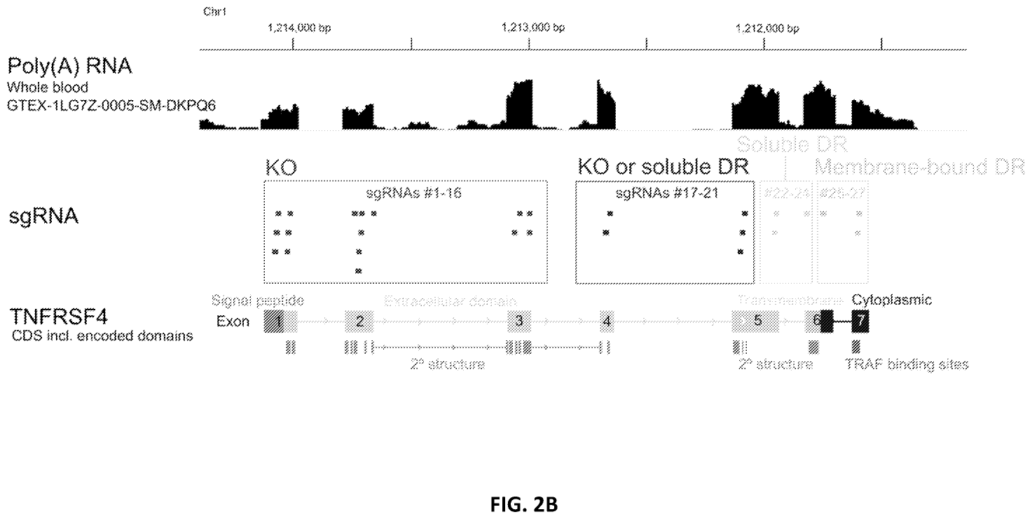

In certain embodiments, any region of an IL1RAP gene (e.g., 5′ untranslated region [UTR], exon 1, exon 2, exon 3, exon 4, exon 5, exon 6, exon 7, exon 8, exon 9, exon 10, exon 11, exon 12, exon 13, exon 14, exon 15, exon 16, exon 17, any intervening intronic regions, intron/exon junctions, the 3′ UTR, or polyadenylation signal) is targeted by an RNA-guided nuclease to alter the gene (see, e.g., FIG. 2A). In some embodiments, the IL1RAP gene targeted by an RNA-guided nuclease is from a mammal. In some embodiments, the IL1RAP gene targeted by an RNA-guided nuclease is from a human (hIL1RAP). In some embodiments, the IL1RAP gene targeted by an RNA-guided nuclease is from a dog (cIL1RAP). In some embodiments, the IL1RAP gene targeted by an RNA-guided nuclease is from a horse (eIL1RAP). In some embodiments, the IL1RAP gene targeted by an RNA-guided nuclease is from a cat (fIL1RAP).

The term “interleukin-1 receptor complex” or “IL1R complex” refers to any number of the protein receptors that comprises the family of transmembrane protein receptors responsible for transmitting or modulating intracellular signaling via binding of the pro-inflammatory cytokine interleukin 1 (IL1). These members include IL1R1, IL1R2, which primarily functions as a decoy receptor, IL1RAP, and IL1RL1, which can complex with other family members in the IL33 or IL36 signaling system.

The terms “Transforming Growth Factor Beta Receptor 1” or “TGFBR1” refer to the genes (NCBI Gene ID: 7046 [human], NCBI Gene ID: 481628 [canine], NCBI Gene ID: 100034117 [equine], NCBI Gene ID: 101094057 [feline]) or an encoded gene product (e.g., UniProt: P36897; NP_004603.1 [human], XP_038538191.1 [canine], XP_023485510.1 [equine], XP_023098269.1 [feline]), as well as sequence variants, proteins harboring conservative amino acid substitutions, and glycoforms thereof. Canonically, the proteins encoded by the genes listed above are transmembrane serine/threonine kinases forming, with TGFBR2, the native receptor for the TGF-beta cytokines TGFB1, TGFB2 and TGFB3. When bound to its ligand, TGFBR1 is phosphorylated by TGFBR2, activating intracellular signaling regulating multiple physiological and pathological processes through release of SMAD2, which can then translocate to the nucleus or activation of other cytoplasmic signaling mediators. In some instances, and merely for the sake of disambiguation, a prefix is added when referring to the protein or gene of a particular species (with h, c, e, and f, referring to the human, canine, equine, and feline forms, respectively).

In certain embodiments, any region of an TGFBR1 gene (e.g., 5′ untranslated region [UTR], exon 1, exon 2, exon 3, exon 4, exon 5, exon 6, exon 7, exon 8, exon 9, exon 10, exon 11, exon 12, any intervening intronic regions, intron/exon junctions, the 3′ UTR, or polyadenylation signal) is targeted by an RNA-guided nuclease to alter the gene. In some embodiments, the TGFBR1 gene targeted by an RNA-guided nuclease is from a mammal. In some embodiments, the TGFBR1 gene targeted by an RNA-guided nuclease is from a human (hTGFBR1). In some embodiments, the TGFBR1 gene targeted by an RNA-guided nuclease is from a dog (cTGFBR1). In some embodiments, the TGFBR1 gene targeted by an RNA-guided nuclease is from a horse (eTGFBR1). In some embodiments, the TGFBR1 gene targeted by an RNA-guided nuclease is from a cat (fTGFBR1).

The terms “Transforming Growth Factor Beta Receptor 2” or “TGFBR2” refer to the genes (NCBI Gene ID: 7048 [human], NCBI Gene ID: 477039 [canine], NCBI Gene ID: 100033860 [equine], NCBI Gene ID: 101091725 [feline]) or an encoded gene product (e.g., UniProt: P37173; NP_003233.4 [human], XP_038288013.1 [canine], XP_023475502.1 [equine], XP_023116415.1 [feline]), as well as sequence variants, proteins harboring conservative amino acid substitutions, and glycoforms thereof Canonically, the proteins encoded by the genes listed above are transmembrane serine/threonine kinases forming, with TGFBR2, the native receptor for the TGF-beta cytokines TGFB1, TGFB2 and TGFB3. When bound to its ligand, TGFBR1 is phosphorylated by TGFBR2, activating intracellular signaling regulating multiple physiological and pathological processes through release of SMAD2, which can then translocate to the nucleus or activation of other cytoplasmic signaling mediators. In some instances, and merely for the sake of disambiguation, a prefix is added when referring to the protein or gene of a particular species (with h, c, e, and f, referring to the human, canine, equine, and feline forms, respectively).

In certain embodiments, any region of an TGFBR2 gene (e.g., 5′ untranslated region [UTR], exon 1, exon 2, exon 3, exon 4, exon 5, exon 6, exon 7, exon 8, exon 9, exon 10, any intervening intronic regions, intron/exon junctions, the 3′ UTR, or polyadenylation signal) is targeted by an RNA-guided nuclease to alter the gene. In some embodiments, the TGFBR2 gene targeted by an RNA-guided nuclease is from a mammal. In some embodiments, the TGFBR2 gene targeted by an RNA-guided nuclease is from a human (hTGFBR2). In some embodiments, the TGFBR2 gene targeted by an RNA-guided nuclease is from a dog (cTGFBR2). In some embodiments, the TGFBR2 gene targeted by an RNA-guided nuclease is from a horse (eTGFBR2). In some embodiments, the TGFBR2 gene targeted by an RNA-guided nuclease is from a cat (fTGFBR2).

The terms “Interleukin-6 Receptor” or “IL6R” refer to the genes (NCBI Gene ID: 3560 [human], NCBI Gene ID: 612271 [canine], NCBI Gene ID: 102148787 [equine], NCBI Gene ID: 101085689 [feline]) or an encoded gene product (e.g., UniProt: P08887; CAA41231.1 [human], XP_038527979.1 [canine], XP_023496854.1 [equine], XP_023103841.2 [feline]), as well as sequence variants, proteins harboring conservative amino acid substitutions, and glycoforms thereof. Canonically, the proteins encoded by the genes listed above are transmembrane proteins capable of binding to interleukin-6, its native ligand. This binding event triggers intracellular signaling events that result in pro-inflammatory responses. See generally, Wolf, J., et al. (2014). Cytokine, 70 (1), 11-20. In some instances, and merely for the sake of disambiguation, a prefix is added when referring to the protein or gene of a particular species (with h, c, e, and f, referring to the human, canine, equine, and feline forms, respectively).

In certain embodiments, any region of an IL6R gene (e.g., 5′ untranslated region [UTR], exon 1, exon 2, exon 3, exon 4, exon 5, exon 6, exon 7, exon 8, exon 9, exon 10, exon 11, exon 12, exon 13, exon 14, exon 15, any intervening intronic regions, intron/exon junctions, the 3′ UTR, or polyadenylation signal) is targeted by an RNA-guided nuclease to alter the gene. In some embodiments, the IL6R gene targeted by an RNA-guided nuclease is from a mammal. In some embodiments, the IL6R gene targeted by an RNA-guided nuclease is from a human (hIL6R). In some embodiments, the IL6R gene targeted by an RNA-guided nuclease is from a dog (cIL6R). In some embodiments, the IL6R gene targeted by an RNA-guided nuclease is from a horse (eIL6R). In some embodiments, the IL6R gene targeted by an RNA-guided nuclease is from a cat (fIL6R).

The terms “Interleukin-6 Cytokine Family Signal Transducer,” “GP130,” or “IL6ST” refer to the genes (NCBI Gene ID: 3572 [human], NCBI Gene ID: 403545 [canine], NCBI Gene ID: 100051700 [equine], NCBI Gene ID: 101089832 [feline]) or an encoded gene product (e.g., UniProt: P40189; NP_001177910.1 [human], NP_001273950.1 [canine], XP_023481030.1 [equine], XP_011281205.1 [feline]), as well as sequence variants, proteins harboring conservative amino acid substitutions, and glycoforms thereof. Canonically, the proteins encoded by the genes listed above are signal transducers shared by many cytokines, including interleukin 6 (IL6), ciliary neurotrophic factor (CNTF), leukemia inhibitory factor (LIF), and oncostatin M (OSM) and function as a part of the cytokine receptor complex. Activation of this protein is dependent upon the binding of cytokines to their receptors (e.g., IL6 to IL6R). Knockout studies in mice suggest that this gene plays a critical role in regulating myocyte apoptosis. See generally, Martínez-Pérez, C., et al. (2021). Journal of Personalized Medicine, 11 (7), 618. In some instances, and merely for the sake of disambiguation, a prefix is added when referring to the protein or gene of a particular species (with h, c, e, and f, referring to the human, canine, equine, and feline forms, respectively

In certain embodiments, any region of an IL6ST gene (e.g., 5′ untranslated region [UTR], exon 1, exon 2, exon 3, exon 4, exon 5, exon 6, exon 7, exon 8, exon 9, exon 10, exon 11, exon 12, exon 13, exon 14, exon 15, exon 16, exon 17, exon 18, exon 19, exon 20, any intervening intronic regions, intron/exon junctions, the 3′ UTR, or polyadenylation signal) is targeted by an RNA-guided nuclease to alter the gene. In some embodiments, the IL6ST gene targeted by an RNA-guided nuclease is from a mammal. In some embodiments, the IL6ST gene targeted by an RNA-guided nuclease is from a human (hIL6ST). In some embodiments, the IL6ST gene targeted by an RNA-guided nuclease is from a dog (cIL6ST). In some embodiments, the IL6ST gene targeted by an RNA-guided nuclease is from a horse (eIL6ST). In some embodiments, the IL6ST gene targeted by an RNA-guided nuclease is from a cat (fIL6ST).

The terms “Tumor Necrosis Factor Receptor 1” or “TNFRSF1A” refer to the genes (NCBI Gene ID: 7132 [human], NCBI Gene ID: 403634 [canine], NCBI Gene ID: 100059548 [equine], NCBI Gene ID: 493957 [feline]) or an encoded gene product (e.g., UniProt: P19438; NP_001056.1 [human], XP_038295153.1 [canine], XP_023498787.1 [equine], NP_001009361.1 [feline]), as well as sequence variants, proteins harboring conservative amino acid substitutions, and glycoforms thereof. Canonically, the proteins encoded by the genes listed above are transmembrane receptor proteins capable of binding Tumor Necrosis Factor Alpha (TNFA) or lymphotoxin alpha (LTA), its principal ligand. Upon binding to TNFA, the receptor trimerizes and is activated, transmitting intracellular signaling cascades with role in various processes, including apoptosis and inflammation. See generally, Ward-Kavanagh, L. K., et al. (2016). Immunity, 44 (5), 1005-1019. In some instances, and merely for the sake of disambiguation, a prefix is added when referring to the protein or gene of a particular species (with h, c, e, and f, referring to the human, canine, equine, and feline forms, respectively).

In certain embodiments, any region of an TNFRSF1A gene (e.g., 5′ untranslated region [UTR], exon 1, exon 2, exon 3, exon 4, exon 5, exon 6, exon 7, exon 8, exon 9, exon 10, exon 11, any intervening intronic regions, intron/exon junctions, the 3′ UTR, or polyadenylation signal) is targeted by an RNA-guided nuclease to alter the gene. In some embodiments, the TNFRSF1A gene targeted by an RNA-guided nuclease is from a mammal. In some embodiments, the TNFRSF1A gene targeted by an RNA-guided nuclease is from a human (hTNFRSF1A). In some embodiments, the TNFRSF1A gene targeted by an RNA-guided nuclease is from a dog (cTNFRSF1A). In some embodiments, the TNFRSF1A gene targeted by an RNA-guided nuclease is from a horse (eTNFRSF1A). In some embodiments, the TNFRSF1A gene targeted by an RNA-guided nuclease is from a cat (fTNFRSF1A).

The terms “Tumor Necrosis Factor Receptor 2” or “TNFRSF1B” refer to the genes (NCBI Gene ID: 7133 [human], NCBI Gene ID: 487437 [canine], NCBI Gene ID: 100055840 [equine], NCBI Gene ID: 101080392 [feline]) or an encoded gene product (e.g., UniProt: P20333; XP_011540362.1 [human], XP_038387905.1 [canine], XP_023491528.1 [equine], XP_023113905.2 [feline]), as well as sequence variants, proteins harboring conservative amino acid substitutions, and glycoforms thereof. Canonically, the proteins encoded by the genes listed above are transmembrane receptor proteins capable of binding TNFA or LTA and are implicated in pro-survival pathways through downstream activation of NFkB pathway. See generally, Ward-Kavanagh, L. K., et al. (2016). Immunity, 44 (5), 1005-1019. In some instances, and merely for the sake of disambiguation, a prefix is added when referring to the protein or gene of a particular species (with h, c, e, and f, referring to the human, canine, equine, and feline forms, respectively).

In certain embodiments, any region of an TNFRSF1B gene (e.g., 5′ untranslated region [UTR], exon 1, exon 2, exon 3, exon 4, exon 5, exon 6, exon 7, exon 8, exon 9, exon 10, exon 11, exon 13, any intervening intronic regions, intron/exon junctions, the 3′ UTR, or polyadenylation signal) is targeted by an RNA-guided nuclease to alter the gene. In some embodiments, the TNFRSF1B gene targeted by an RNA-guided nuclease is from a mammal. In some embodiments, the TNFRSF1B gene targeted by an RNA-guided nuclease is from a human (hTNFRSF1B). In some embodiments, the TNFRSF1B gene targeted by an RNA-guided nuclease is from a dog (cTNFRSF1B). In some embodiments, the TNFRSF1B gene targeted by an RNA-guided nuclease is from a horse (eTNFRSF1B). In some embodiments, the TNFRSF1B gene targeted by an RNA-guided nuclease is from a cat (fTNFRSF1B).

The terms “Lymphotoxin Beta Receptor” or “TNFRSF3” refer to the genes (NCBI Gene ID: 4055 [human], NCBI Gene ID: 486728 [canine], NCBI Gene ID: 100059650 [equine], NCBI Gene ID: 101081146 [feline]) or an encoded gene product (e.g., UniProt: P36941; NP_001257916.1 [human], XP_038295148.1 [canine], XP_001492220.3 [equine], XP_003988366.4 [feline]), as well as sequence variants, proteins harboring conservative amino acid substitutions, and glycoforms thereof. Canonically, the proteins encoded by the genes listed above play a role in signaling during the development of lymphoid and other organs, lipid metabolism, immune response, and programmed cell death. Major ligands of this receptor include lymphotoxin alpha/beta and tumor necrosis factor ligand superfamily member 14 (TNFSF14). Activity of this receptor has also been linked to carcinogenesis. See generally, Seymour, R., et al. (2006). Veterinary Pathology, 43 (4), 401-423. In some instances, and merely for the sake of disambiguation, a prefix is added when referring to the protein or gene of a particular species (with h, c, e, and f, referring to the human, canine, equine, and feline forms, respectively).

In certain embodiments, any region of an TNFRSF3 gene (e.g., 5′ untranslated region [UTR], exon 1, exon 2, exon 3, exon 4, exon 5, exon 6, exon 7, exon 8, exon 9, exon 10, exon 11, exon 12, any intervening intronic regions, intron/exon junctions, the 3′ UTR, or polyadenylation signal) is targeted by an RNA-guided nuclease to alter the gene. In some embodiments, the TNFRSF3 gene targeted by an RNA-guided nuclease is from a mammal. In some embodiments, the TNFRSF3 gene targeted by an RNA-guided nuclease is from a human (hTNFRSF3). In some embodiments, the TNFRSF3 gene targeted by an RNA-guided nuclease is from a dog (cTNFRSF3). In some embodiments, the TNFRSF3 gene targeted by an RNA-guided nuclease is from a horse (eTNFRSF3). In some embodiments, the TNFRSF3 gene targeted by an RNA-guided nuclease is from a cat (fTNFRSF3).

The terms “OX40 Receptor,” “OX40,” “CD134,” or “TNFRSF4” refer to the genes (NCBI Gene ID: 7293 [human], NCBI Gene ID: 489600 [canine], NCBI Gene ID: 100066167 [equine], NCBI Gene ID: 493665 [feline]) or an encoded gene product (e.g., UniProt: P43489; XP_011540377.1 [human], XP_038520220.1 [canine], XP_001503612.3 [equine], NP_001009200.1 [feline]), as well as sequence variants, proteins harboring conservative amino acid substitutions, and glycoforms thereof. Canonically, the proteins encoded by the genes listed above specifically bind to OX40 ligand (OX40L) and are predominantly expressed on lymphocytes. The result of this binding is transient downstream intracellular signaling mediated via TRAF2 and other effector proteins that often occurs in response to the presence of antigen. See generally, Ward-Kavanagh, L. K., et al. (2016). Immunity, 44 (5), 1005-1019. In some instances, and merely for the sake of disambiguation, a prefix is added when referring to the protein or gene of a particular species (with h, c, e, and f, referring to the human, canine, equine, and feline forms, respectively).

In certain embodiments, any region of an TNFRSF4 gene (e.g., 5′ untranslated region [UTR], exon 1, exon 2, exon 3, exon 4, exon 5, exon 6, exon 7, any intervening intronic regions, intron/exon junctions, the 3′ UTR, or polyadenylation signal) is targeted by an RNA-guided nuclease to alter the gene. In some embodiments, the TNFRSF4 gene targeted by an RNA-guided nuclease is from a mammal. In some embodiments, the TNFRSF4 gene targeted by an RNA-guided nuclease is from a human (hTNFRSF4). In some embodiments, the TNFRSF4 gene targeted by an RNA-guided nuclease is from a dog (cTNFRSF4). In some embodiments, the TNFRSF4 gene targeted by an RNA-guided nuclease is from a horse (eTNFRSF4). In some embodiments, the TNFRSF4 gene targeted by an RNA-guided nuclease is from a cat (fTNFRSF4).

The terms “TNF Receptor Superfamily Member 11A” or “TNFRSF11A” refer to the genes (NCBI Gene ID: 8792 [human], NCBI Gene ID: 483957 [canine], NCBI Gene ID: 100056617 [equine], NCBI Gene ID: 101090651 [feline]) or an encoded gene product (e.g., UniProt: Q9Y6Q6; NP_001257878.1 [human], XP_038509502.1 [canine], XP_023503703.1 [equine], XP_023096972.1 [feline]), as well as sequence variants, proteins harboring conservative amino acid substitutions, and glycoforms thereof. Canonically, the proteins encoded by the genes listed above are transmembrane proteins capable of interacting with n various TRAF family proteins, through which the receptors induce the activation of NF-kappa B and MAPK8/JNK pathways. This receptor, with its ligand, is an important regulator of the interaction between T cells and dendritic cells and is also an essential mediator for bone metabolism and development. See generally, Xue, J. Y., et al. (2021). Journal of Bone and Mineral Metabolism, 39 (1), 45-53. In some instances, and merely for the sake of disambiguation, a prefix is added when referring to the protein or gene of a particular species (with h, c, e, and f, referring to the human, canine, equine, and feline forms, respectively).

In certain embodiments, any region of an TNFRSF11A gene (e.g., 5′ untranslated region [UTR], exon 1, exon 2, exon 3, exon 4, exon 5, exon 6, exon 7, exon 8, exon 9, exon 10, exon 11, exon 12, any intervening intronic regions, intron/exon junctions, the 3′ UTR, or polyadenylation signal) is targeted by an RNA-guided nuclease to alter the gene. In some embodiments, the TNFRSF11A gene targeted by an RNA-guided nuclease is from a mammal. In some embodiments, the TNFRSF11A gene targeted by an RNA-guided nuclease is from a human (hTNFRSF11A). In some embodiments, the TNFRSF11A gene targeted by an RNA-guided nuclease is from a dog (cTNFRSF11A). In some embodiments, the TNFRSF11A gene targeted by an RNA-guided nuclease is from a horse (eTNFRSF11A). In some embodiments, the TNFRSF11A gene targeted by an RNA-guided nuclease is from a cat (fTNFRSF11A).

The term “treatment” refers to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. For example, a composition, method, or system of the present disclosure may be administered as a prophylactic treatment to a subject that has a predisposition for a given condition (e.g., arthritis). “Treatment”, as used herein, covers any treatment of a disease in a mammal, particularly in a human, canine, feline, or equine, and includes: (a) preventing the disease from occurring in a subject which may be predisposed to the disease but has not yet been diagnosed as having it; (b) inhibiting the disease, i.e., arresting its development or progression; and (c) relieving the disease, i.e., causing regression of the disease and/or relieving one or more disease symptoms.

“Treatment” is also meant to encompass delivery of an agent in order to provide for a pharmacologic effect, even in the absence of a disease or condition. For example, “treatment” encompasses delivery of a composition that can elicit an immune response or confer immunity in the absence of a disease condition, e.g., in the case of a vaccine. It is understood that compositions and methods of the present disclosure are applicable to treat all mammals, including, but not limited to human, canine, feline, equine, and bovine subjects.

The term “therapeutically effective” refers to the amount of a composition or combination of compositions as described herein that is sufficient to effect the intended application including, but not limited to, disease treatment. A therapeutically effective amount may vary depending upon the intended application (in vitro or in vivo), or the subject and disease condition being treated (e.g., the weight, age and gender of the subject), the severity of the disease condition, or the manner of administration. The term also applies to a dose that will induce a particular response in target cells (e.g., the reduction of platelet adhesion and/or cell migration). The specific dose will vary depending on the particular composition(s) chosen, the dosing regimen to be followed, whether the composition is administered in combination with other compositions or compounds, timing of administration, the tissue to which it is administered, and the physical delivery system in which the composition is carried.

The term “joint disease” is defined as measurable abnormalities in the cells or tissues of the joint that could lead to illness, for example, metabolic and molecular derangements triggering anatomical and/or physiological changes in the joint. Including, but not limited to, radiographic detection of joint space narrowing, subchondral sclerosis, subchondral cysts, and osteophyte formation.

“Joint illness” is defined in human subjects as symptoms that drive the subject to seek medical intervention, for example, subject reported pain, stiffness, swelling, or immobility. For non-human mammals, “joint illness” is defined, for example, as lameness, observable changes in gait, weight bearing, allodynia, or exploratory behavior.

A “back or spine condition or disorder” includes, but is not limited to, low back pain, neck pain, discogenic disorders, adolescent idiopathic scoliosis, adult degenerative scoliosis, cervical degenerative disc disease, cervical disc herniation, cervical myelopathy, cervical stenosis, compression fractures, degenerative spondylolisthesis, isthmic spondylolisthesis, low back sprains and strains, lumbar degenerative disc disease, lumbar disc herniation, lumbar stenosis, neck sprain (whiplash) and strain, neck strain, osteoporosis, and whiplash. Generally, such disorders or conditions contribute to or cause localized nociception, inflammation, or morphological changes (e.g., fibrosis, degeneration, osteolysis, osteogenesis) at the cervical, thoracic, lumbar or sacral spine, or surrounding tissues.

“Low back pain” is defined as measurable or discernible pain or discomfort (either chronic or sporadic) in a given subject, encompassing at least the lumbar-spinal region of a mammal. The pain may present as being localized to the lower back (e.g., muscle ache) or as shooting, burning, stinging, and/or radiating sensations throughout the subject's back and/or extremities. The pain may be idiopathic or may be associated with one or more (diagnosed or undiagnosed) underlying conditions including, but not limited to, chronic inflammation, arthritis, osteoporosis, trauma (e.g., post-surgical), neuropathies, musculo-skeletal abnormalities (e.g., slipped discs or spinal stenosis), herniated nucleus pulposus (HNP), annular ligament tears, facet joint arthritis, radicular nerve compression, and/or degenerative disorders.

“Neck pain” is defined as measurable or discernable pain or discomfort associated with the cervical spine or adjacent ligaments, muscles, and/or tendons. The pain may manifest as localized pain in the neck or shooting, stinging, burning, and/or radiating sensations throughout the back or extremities, including, but not limited to, the subject's head, shoulders, arms, legs, and/or back. Neck pain may be idiopathic or associated with one or more (diagnosed or undiagnosed) underlying conditions, including, but not limited to, rheumatoid arthritis, osteoporosis, fibromyalgia, chronic inflammation, herniated disc, spondylosis, spinal stenosis, whiplash, and/or degenerative disorders.

The terms “polynucleotide,” “nucleotide,” and “nucleic acid” are used interchangeably herein to refer to all forms of nucleic acid, oligonucleotides, including deoxyribonucleic acid (DNA) and ribonucleic acid (RNA). Polynucleotides include genomic DNA, cDNA and antisense DNA, and spliced or unspliced mRNA, rRNA, tRNA, lncRNA, RNA antagomirs, and inhibitory DNA or RNA (RNAi, e.g., small or short hairpin (sh) RNA, microRNA (miRNA), aptamers, small or short interfering (si) RNA, trans-splicing RNA, or antisense RNA). Polynucleotides also include non-coding RNA, which include for example, but are not limited to, RNAi, miRNAs, lncRNAs, RNA antagomirs, aptamers, and any other non-coding RNAs known to those of skill in the art. Polynucleotides include naturally occurring, synthetic, and intentionally altered or modified polynucleotides as well as analogues and derivatives. The term “polynucleotide” also refers to a polymeric form of nucleotides of any length, including deoxyribonucleotides or ribonucleotides, or analogs thereof, and is synonymous with nucleic acid sequence. A polynucleotide may comprise modified nucleotides, such as methylated nucleotides and nucleotide analogs, and may be interrupted by non-nucleotide components. If present, modifications to the nucleotide structure may be imparted before or after assembly of the polymer. The term polynucleotide, as used herein, refers interchangeably to double- and single-stranded molecules. Unless otherwise specified or required, any embodiment as described herein encompassing a polynucleotide encompasses both the double-stranded form and each of two complementary single-stranded forms known or predicted to make up the double-stranded form. Polynucleotides can be single, double, or triplex, linear or circular, and can be of any length. In discussing polynucleotides, a sequence or structure of a particular polynucleotide may be described herein according to the convention of providing the sequence in the 5′ to 3′ direction.

The term “gene” or “nucleotide sequence encoding a polypeptide” refers to the segment of DNA involved in producing a polypeptide chain. The DNA segment may include regions preceding and following the coding region (leader and trailer) involved in the transcription/translation of the gene product and the regulation of the transcription/translation, as well as intervening sequences (introns) between individual coding segments (exons). For example, a gene includes a polynucleotide containing at least one open reading frame capable of encoding a particular protein or polypeptide after being transcribed and translated.

The terms “extracellular domain” and “ectodomain” may be used interchangeably and, when referring to transmembrane cellular receptors, is defined as the portion of the protein that is exposed to the extracellular environment and is able to engage with and/or bind a ligand.

The terms “cytoplasmic domain” and “intracellular domain” may be used interchangeably and, when referring to transmembrane receptors, define the portion of the protein that is exposed to the cytoplasm. In many instances, these portions of the proteins comprise signaling domains to recruit and associate with various intracellular factors. Following engagement with a ligand via the extracellular domain, the interaction effects changes that may result in new association, dissociation or recruitment of various cytoplasmic factors that aid in transducing a signal.

The term “transmembrane domain,” which may be abbreviated as “TM,” as it refers to transmembrane receptors, is defined as the portion of the protein is embedded within the plasma membrane (i.e., not exposed to either the extracellular environment or the cytosol). Transmembrane domains are generally of a more hydrophobic character than either the extracellular or cytoplasmic portions and often adopt higher order helical structures. Though its primary role is an anchor, ligand-induced conformational changes to particular receptors have been shown to impact the transmembrane domain such that it is integral to the subsequent intracellular signaling.

The term “receptor” refers to a protein capable of binding another cognate protein (i.e., its ligand) with high affinity. This receptor-ligand interaction may be 1:1, or result in multimerization, wherein numerous proteins aggregate to bind one or more ligands. Receptors are generally present at the cell surface, such that they may most efficiently encounter a ligand and initiate intracellular signaling.

The term “intracellular signaling” refers to cellular changes that result due to events occurring at the cell surface. Typically, a soluble ligand binds its receptor at the cell surface, which can induce changes in the receptor, such that associated intracellular factors are also affected. These factors may then impact others within the cell, and this cascade continues until, in many cases, a particular factor is able to alter gene expression in the nucleus in response to the stimulus at the surface.

The term “RNA-guided nuclease” refers to an enzyme capable of breaking the backbone of, for example, a DNA molecule. The activity of RNA-guided nucleases is directed by a nucleic acid molecule (i.e., guide RNA). Once properly oriented to form a functional ribonucleoprotein complex, the enzyme locates a specific position within a target nucleic acid (e.g., a gene or locus) via sequence complementarity with a portion of the guide RNA. Non-exhaustive examples of RNA-guided nucleases include Cas9, Cas12 and Cas12a (previously known as Cpf1).

The term “Cas9” refers to an RNA-guided, double-stranded DNA-binding nuclease protein or nickase protein, or a variant thereof and may be used to refer to either naturally-occurring or recombinant Cas9 nucleases variants (e.g., ES-Cas9, HF-Cas9, PE-Cas9, and AR-Cas9). The wildtype Cas9 nuclease has two functional domains, e.g., RuvC and HNH, that simultaneously cut both strands of double stranded DNA, resulting in a double-strand break. Cas9 enzymes described herein may comprise a HNH or HNH-like nuclease domain and/or a RuvC or RuvC-like nuclease domain without impacts on the ability to induce double-strand breaks in genomic DNA (e.g., at a target locus) when both functional domains are active. The Cas9 enzyme may comprise one or more catalytic domains of a Cas9 protein derived from bacteria belonging to the group consisting of Corynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, and Campylobacter. In some embodiments, the two catalytic domains are derived from different bacteria species.

As used herein, “PAM” refers to a Protospacer Adjacent Motif and is necessary for an RNA-guided nuclease to bind a target nucleic acid. In many instances, the PAM directly abuts the complementary sequence in the target. Naturally-occurring Cas9, for example, molecules recognize specific PAM sequences (see, e.g., Table 1). In some embodiments, a Cas9 molecule has the same PAM specificities as a naturally occurring Cas9 molecule. In other embodiments, a Cas9 molecule has a PAM specificity not associated with a naturally occurring Cas9 molecule. In other embodiments, a Cas9 molecule's PAM specificity is not associated with the naturally occurring Cas9 molecule to which it has the closest sequence homology. For example, a naturally occurring Cas9 molecule can be altered such that the PAM sequence recognition is altered to decrease off target sites, improve specificity, or eliminate a PAM recognition requirement. In an embodiment, a Cas9 molecule may be altered (e.g., to lengthen a PAM recognition sequence, improve Cas9 specificity to high level of identity, to decrease off target sites, and/or increase specificity). In an embodiment, the length of the PAM recognition sequence is at least 4, 5, 6, 7, 8, 9, 10 or 15 amino acids in length. In some embodiments, a Cas9 molecule may be altered to ablate PAM recognition.

The terms “guide RNA,” “gRNA” or “sgRNA” may be used interchangeably and refer to an RNA molecule, preferably a synthetic RNA molecule, composed of a targeting (crRNA) sequence and scaffold. These molecules, once loaded onto a functional RNA-guided nuclease can direct sequence-specific cleavage of a target nucleic acid.

An sgRNA can be administered or formulated, e.g., as a synthetic RNA, or as a nucleic acid comprising a sequence encoding the gRNA, which is then expressed in the target cells. As would be evident to one of ordinary skill in the art, various tools may be used in the design and/or optimization of an sgRNA in order to, for example, increase specificity and/or precision of genomic editing at a particular site.

In general, candidate sgRNAs may be designed and identified by first locating suitable PAMs within a genomic sequence. Then additional calculations may be utilized to predict on-target and off-target efficiencies. Available web-based tools to aid in the initial design and modeling of candidate sgRNAs include, without limitation, CRISPRseek, CRISPR Design Tool, Cas-OFFinder, E-CRISP, ChopChop, CasOT, CRISPR direct, CRISPOR, BREAKING-CAS, CrispRGold, and CCTop. See, e.g., Safari, F. et al. (2017). Current Pharmaceutical Biotechnology, 18 (13): 1038-54, which is incorporated by reference herein in its entirety for all purposes. Such tools are also described, for example, in PCT Publication No. WO2014093701A1 and Liu, G. et al. (2020). Computational approaches for effective CRISPR guide RNA design and evaluation. Computational and Structural Biotechnology Journal, 18:35-44, each of which is incorporated by reference herein in its entirety for all purposes. Candidate sgRNAs may be further assessed by experimental screening or other methodologies.

The terms “CRISPR RNA” or “crRNA” refer to the portion of an sgRNA molecule with complementarity to the target nucleic acid.

The phrase “pharmaceutically acceptable” refers to those compounds, materials, compositions, and/or dosage forms that are, within the scope of sound medical judgment, suitable for use in contact with the tissues of human beings and animals without excessive toxicity, irritation, allergic response, or other problems or complications commensurate with a reasonable benefit/risk ratio.

The terms “pharmaceutically acceptable carrier” or “pharmaceutically acceptable excipient” are intended to include any and all solvents, dispersion media, coatings, antibacterial and antifungal agents, isotonic and absorption delaying agents, and inert ingredients. The use of such pharmaceutically acceptable carriers or pharmaceutically acceptable excipients for active pharmaceutical ingredients is well known in the art. Except insofar as any conventional pharmaceutically acceptable carrier or pharmaceutically acceptable excipient is incompatible with the active pharmaceutical ingredient, its use in the therapeutic compositions of the disclosure is contemplated. Additional active pharmaceutical ingredients, such as other drugs, can also be incorporated into the described compositions and methods.

The term “pharmaceutically acceptable excipient” is intended to include vehicles and carriers capable of being co-administered with a compound to facilitate the performance of its intended function. The use of such media for pharmaceutically active substances is well known in the art. Examples of such vehicles and carriers include solutions, solvents, dispersion media, delay agents, emulsions and the like. Any other conventional carrier suitable for use with the multi-binding compounds also falls within the scope of the present disclosure.

As used herein, the term “a”, “an”, or “the” generally is construed to cover both the singular and the plural forms.

The terms “about” and “approximately” mean within a statistically meaningful range of a value. Such a range can be within an order of magnitude, preferably within 50%, more preferably within 20%, more preferably still within 10%, and even more preferably within 5% of a given value or range. The allowable variation encompassed by the terms “about” or “approximately” depends on the particular system under study, and can be readily appreciated by one of ordinary skill in the art. Moreover, as used herein, the terms “about” and “approximately” mean that compositions, amounts, formulations, parameters, shapes and other quantities and characteristics are not and need not be exact, but may be approximate and/or larger or smaller, as desired, reflecting tolerances, conversion factors, rounding off, measurement error and the like, and other factors known to those of skill in the art. In general, a dimension, size, formulation, parameter, shape or other quantity or characteristic is “about” or “approximate,” whether or not expressly stated to be such. It is noted that embodiments of very different sizes, shapes and dimensions may employ the described arrangements.

The term “substantially” as used herein can refer to a majority of, or mostly, as in at least about 50%, 60%, 70%, 80%, 90%, 95%, 96%, 97%, 98%, 99%, 99.5%, 99.9%, 99.99%, or at least about 99.999% or more.

The transitional terms “comprising,” “consisting essentially of,” and “consisting of,” when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinary associated with the specified material(s). The term “consisting essentially of” limits the scope of a claim to the specified elements, steps or material(s) and those that do not materially affect the basic and novel characteristic(s) of the claimed methods and compositions. All compositions, methods, and kits described herein that embody the present disclosure can, in alternate embodiments, be more specifically defined by any of the transitional terms “comprising,” “consisting essentially of,” and “consisting of.”

### III. Methods

### A. CRISPR

Unless otherwise defined, all technical and scientific terms used herein have the same meaning as commonly understood by one of ordinary skill in the art to which this disclosure belongs. Although methods and materials similar or equivalent to those described herein may be used in the practice or testing of the present disclosure, suitable methods and materials are described below. All publications, patent applications, patents, and other references mentioned herein are incorporated by reference in their entirety. In addition, the materials, methods, and examples are illustrative only and not intended to be limiting.

In one aspect, the present disclosure encompasses compositions relating to clustered regularly interspaced short palindromic repeats (CRISPR) and CRISPR-associated RNA-guided nucleases and associated methods, components, and compositions (hereafter, CRISPR/Cas systems). Such systems minimally require at least one isolated or non-naturally-occurring RNA-guided nuclease (e.g., a Cas9 protein) and at least one isolated or non-naturally-occurring guide RNA (e.g., an sgRNA) to effectuate augmentation of a nucleic acid sequence (e.g., genomic DNA).

In some embodiments, a CRISPR/Cas system effectuates the alteration of a targeted gene or locus in a eukaryotic cell by effecting an alteration of the sequence at a target position (e.g., by creating an insertion or deletion (collectively, an indel) resulting in loss-of-function of (i.e., knocking out) the affected gene or allele; e.g., a nucleotide substitution resulting in a truncation, nonsense mutation, or other type of loss-of-function of an encoded product of, for example, one or more IL1R1, IL1RAP, TGFBR1, TGFBR2, IL6R, IL6ST, TNFRSF1A, TNFRSF1B, TNFRSF3, TNFRSF4, or TNFRSF11A gene (i.e., mRNA or protein); a deletion of one or more nucleotides resulting in a truncation, nonsense mutation, or other type of loss-of-function of an encoded product of, for example, one or more IL1R1, IL1RAP, TGFBR1, TGFBR2, IL6R, IL6ST, TNFRSF1A, TNFRSF1B, TNFRSF3, TNFRSF4, or TNFRSF11A gene; e.g., loss-of-function of the encoded mRNA or protein by a single nucleotide, double nucleotide, or other frame-shifting deletion, or a deletion resulting in a premature stop codon; or an insertion resulting in a truncation, nonsense mutation, or other type of loss-of-function of an encoded gene product, such as an encoded gene product of, for example, one or more IL1R1, IL1RAP, TGFBR1, TGFBR2, IL6R, IL6ST, TNFRSF1A, TNFRSF1B, TNFRSF3, TNFRSF4, or TNFRSF11A gene (i.e., mRNA or protein); e.g., a single nucleotide, double nucleotide, or other frame-shifting insertions, or an insertion resulting in a premature stop codon. In some embodiments, a CRISPR/Cas system of the present disclosure provides for the alteration of a gene and/or encoded product of a gene, such that the altered product has a resultant loss-of-function and becomes a dominant negative or decoy (e.g., a transmembrane receptor incapable of initiating intracellular signaling or a soluble receptor).

In one aspect, CRISPR/Cas systems effectuate changes to the sequence of a nucleic acid through nuclease activity. For example, in the case of genomic DNA, the RNA-guided-nuclease locates a target position within a targeted gene or locus by sequence complementarity with the target genomic sequence (e.g., CRISPR RNA (crRNA) or a complementary component of a synthetic single guide RNA (sgRNA)) and cleaves the genomic DNA upon recognition of a particular, nuclease-specific motif called the protospacer adjacent motif (PAM). See generally, Collias, D., & Beisel, C. L. (2021). Nature Communications, 12 (1), 1-12.

Nuclease activity (i.e., cleavage) induces a double-strand break (DSB) in the case of genomic DNA. Endogenous cellular mechanisms of DSB repair, namely non-homologous end joining (NHEJ), microhomology-mediated end joining (MMEJ), and homologous recombination, result in erroneous repair at a given target position with some calculable frequency as a result of interference from said components of the CRISPR/Cas system, thereby introducing substitutions or indels into the genomic DNA. See generally Scully, R., et al. (2019). Nature Reviews Molecular Cell Biology, 20 (11), 698-714. At some frequency, these indels and/or substitutions may result in frameshifts, nonsense mutations (i.e., early stop codons) or truncations that impact the availability of gene products, such as mRNA and/or protein. In certain embodiments, the CRISPR/Cas system may induce a homology-directed repair (HDR) mechanism leading to insertions of non-random sequences at a target position through the use of templates (e.g., an HDR template) provided to the cell as part of the system along with the nuclease and gRNA. See Bloh, K., & Rivera-Torres, N. (2021). International Journal of Molecular Sciences, 22 (8), 3834.

In general, the minimum requirements of the CRISPR/Cas system will be dependent upon the nuclease (i.e., Cas protein) provided therewith. To this extent, these bacterially-derived nucleases have been functionally divided into Types I, III, and V, which all fall into Class 1 and Types II, IV, and VI that are grouped into Class 2.

### Class 1 CRISPR/Cas Systems:

The exact components, compositions, and methods for effectuating a change in a targeted nucleic acid sequence using a Class 1 CRISPR/Cas system will vary, but should minimally include: a nuclease (selected from at least Types I, and III), at least one guide RNA selected from 1) sgRNA or 2) a combination of crRNA and tracrRNA. These CRISPR/Cas systems have been categorized together as Class 1 CRISPR/Cas systems due to their similarities in requirements and mode of action within a eukaryotic cell. To this end, compositions, components, and methods among Class 1 constituents may be considered functionally interchangeable, and the following details, provided merely for exemplary purposes, do not represent an exhaustive list of class members:

Cas3 (see Table 1) is the prototypical Type I DNA nuclease that functions as the effector protein as part of a larger complex (the Cascade complex comprising Cse1, Cse2,), that is capable of genome editing. See generally He, L., et al. (2020). Genes, 11 (2), 208. Unlike other CRISPR/Cas systems, Type I systems localize to the DNA target without the Cas3 nuclease via the Cascade complex, which then recruits Cas3 to cleave DNA upon binding and locating the 3′ PAM. The Cascade complex is also responsible for processing crRNAs such that they can be used to guide it to the target position. Because of this functionality, Cascade has the ability to process multiple arrayed crRNAs from a single molecule. See. Luo, M. (2015). Nucleic Acids Research, 43 (1), 674-681. As such, Type I system may be used to edit multiple targeted genes or loci from a single molecule.

Because the natural Cas3 substrate is ssDNA, its function in genomic editing is thought to be as a nickase; however, when targeted in tandem, the resulting edit is a result of blunt end cuts to opposing strands to approximate a blunt-cutting endonuclease, such as Cas9. See Pickar-Oliver, A., & Gersbach, C. A. (2019). Nature Reviews Molecular Cell Biology, 20 (8), 490-507.

Like Type I nucleases, the Type III system relies upon a complex of proteins to effect nucleic acid cleavage. Particularly, Cas 10 possesses the nuclease activity to cleave ssDNA in prokaryotes. See Tamulaitis, G. Trends in Microbiology, 25 (1), 49-61 (2017). Interestingly, this CRISPR/Cas system, native to archaea, exhibits dual specificity and targets both ssDNA and ssRNA. Aside from this change, the system functions much like Type I in that the crRNA targets an effector complex (similar to Cascade) in a sequence-dependent manner. Similarly, the effector complex processes crRNAs prior to association. The dual nature of this nuclease makes its applications to genomic editing potentially more powerful, as both genomic DNA and, in some cases, mRNAs with the same sequence may be targeted to silence particular targeted genes.

### Class 2 CRISPR/Cas Systems:

The exact components, compositions, and methods for effectuating a change in a targeted nucleic acid sequence using a Class 2 CRISPR/Cas system will vary but should minimally include: a nuclease (selected from at least Types II, and V), at least one guide RNA selected from 1) sgRNA or 2) a combination of crRNA and tracrRNA. These CRISPR/Cas systems have been categorized together as Class 2 CRISPR/Cas systems due to their similarities in requirements and mode of action within a eukaryotic cell. To this end, compositions, components, and methods among Class 2 constituents may be considered functionally interchangeable, and the following details, provided merely for exemplary purposes, do not represent an exhaustive list of class members:

Type II nucleases are the best-characterized CRISPR/Cas systems, particularly the canonical genomic editing nuclease Cas9 (see Table 1). Multiple Cas9 proteins, derived from various bacterial species, have been isolated. The primary distinction between these nucleases is the PAM, a required recognition site within the targeted dsDNA. After association with a gRNA molecule, the crRNA (or targeting domain of a sgRNA) orients the nuclease at the proper position, but the protein's recognition of the PAM is what induces a cleavage event near that site, resulting in a blunt DSB.

In addition to the naturally-derived Cas9 proteins, several engineered variants have similarly been reported. These range from Cas9 with enhanced specific (i.e., less off-target activity), such as espCas9. Others have been catalytically modified via point mutations in the RuvC (e.g., D10A) and HNH (e.g., H840A) domains such that they induce only single-strand breaks (i.e., Cas9 nickases). See Frock, R. et al. (2015). Nature Biotechnology, 33 (2), 179-186. These have also been shown to be less error-prone in editing. Such mitigation of off-target effects becomes paramount when selecting for a desired insertion (i.e., a knock in mutation, in which a desired nucleotide sequence is introduced into a target nucleic acid molecule) rather than a deletion. Indeed, less off-target effects may aid in the preferred DNA repair mechanism (HDR, in most instances for knock in mutations). See generally Naeem, M., et al. (2020). Cells, 9 (7), 1608.

Additional exemplary further engineered variants of canonical Cas proteins (e.g., mutants, chimeras, and include the following (each of which are hereby incorporated by reference in their entireties for all purposes): WO2015035162A2, WO2019126716A1, WO2019126774A1, WO2014093694A1, WO2014150624A1, US20190225955A1, U.S. patent Ser. No. 11/427,818, U.S. patent Ser. No. 11/242,542, U.S. patent Ser. No. 11/098,297, U.S. patent Ser. No. 10/876,100, U.S. patent Ser. No. 10/767,193, U.S. patent Ser. No. 10/494,621, and U.S. patent Ser. No. 10/100,291.

For the avoidance of doubt, SpCas9 collectively refers to any one of the group consisting of espCas9 (also referred to herein as ES-Cas9 or esCas9), HF-Cas9, PE-Cas9, ARCas9 (also referred to as AR-Cas9).

Like the canonical Cas9 systems, Type V nucleases only require a synthetic sgRNA with a targeting domain complementary to a genomic sequence to carry out genomic editing. These nucleases contain a RuvC domain but lack the HNH domain of Type II nucleases. Further, Cas12, for example, leaves a staggered cut in the dsDNA substrate distal to the PAM, as compared to Cas9's blunt cut next to the PAM. Both Cas12a, also known as Cpf1, and Cas12b, also known as C2c1 (see Table 1), act as part of larger complex of two gRNA-associated nucleases that acts on dsDNA as a quaternary structure, nicking each strand simultaneously. See Zetsche, B. et al. (2015). Cell, 163 (3): 759-771; see also Liu, L. et al. (2017). Molecular Cell, 65 (2): 310-322. Additionally, Cas12b (C2cl) is a highly accurate nuclease with little tolerance for mismatches. See Yang, H. et al. (2016). Cell, 167 (7): 1814-1828.e12.

TABLE 1
Exemplary list of Cas nucleases and their requirements

| | | | Spacer |
|---|---|---|---|
| | | Type of end generated | length |
| Nuclease (Species) | PAM (5′ → 3′) | (nucleic acid target) | (nt) |
| Cas9 (S. pyogenes) | NGG | Blunt (dsDNA) | 20 |
| Cas9 (S. aureus) | NNGRRT | Blunt (dsDNA) | 20 |
| Cas9 (C. jejuni) | NNNNRYAC | Blunt (dsDNA) | 22 |
| Cas9 (S. thermophilus) | NNAGAAW | Blunt (dsDNA) | 20 |
| Cas9 (N. meningitidis) | NNNNGATT | Blunt (dsDNA) | 24 |
| Cas9 (F. novicida) | NGG | Blunt (dsDNA) | 21 |
| Cas12a (L. bacterium) | TTTV | 5' staggered | 23-25 |
| | | (dsDNA/ssDNA) | |
| Cas 12a (Acidamino- | TTTN | 5' staggered | 24 |
| coccus sp.) | | (dsDNA/ssDNA) | |
| Cas3 (E. coli) | CTT/CCT/CAT/CTC | None/blunt (ssDNA) | 32 |

See generally Wang, J., Zhang, C., & Feng, B. (2020). Journal of Cellular and Molecular Medicine, 24(6), 3256-3270, where N = any nucleotide; R = any purine (A or G); Y = any pyrimidine (C or T); W = A or T; V = A, C or G.

In one aspect, the CRISPR/Cas system of the present disclosure comprises at least one RNA-guided nuclease (e.g. a Cas protein) derived from one or more of the following selected bacterial genera: Corynebacterium, Sutterella, Legionella, Treponema, Filifactor, Eubacterium, Streptococcus, Lactobacillus, Mycoplasma, Bacteroides, Flavobacterium, Spirochaeta, Azospirillum, Gluconacetobacter, Neisseria, Roseburia, Parvibaculum, Nitratifractor, Campylobacter, Pseudomonas, Streptomyces, Staphylococcus, Francisella, Acidaminococcus, Lachnospiraceae, Leptotrichia, and Prevotella. In some embodiments, the Cas protein is derived from Deltaproteobacteria or Planctomycetes bacterial species.

Some aspects of the present disclosure provide strategies, methods, compositions, and treatment modalities for altering a targeted sequence within a gene locus (e.g., altering the sequence of wild type and/or of a mutant sequence within a cell or within a mammal) by insertion or deletion of one or more nucleotides mediated by an RNA-guided nuclease and one or more guide RNAs (gRNAs), resulting in loss of function of the targeted gene product. In some embodiments, the loss of function results in “knocking out” the gene of interest (i.e., generation of a “knock out”) by ablating gene expression. In some embodiments, the loss function results in a non-functional gene product (i.e., a gene product without all functionality of the wildtype gene product). In some embodiments, the loss of function results in expression of gene product with different characteristics (e.g., different binding affinity or different cellular localization).

In certain embodiments, the targeted gene is selected from IL1R1, IL1RAP, TGFBR1, TGFBR2, IL6R, IL6ST, TNFRSF1A, TNFRSF1B, TNFRSF3, TNFRSF4, TNFRSF11A, and combinations thereof. In some embodiments, any region of the targeted gene (e.g., a promoter region, a 5′ untranslated region, a 3′ untranslated region, an exon, an intron, or an exon/intron border) is targeted by an RNA-guided nuclease to alter the gene. In some embodiments, a non-coding region of the targeted gene (e.g., an enhancer region, a promoter region, an intron, 5′ UTR, 3′ UTR, polyadenylation signal) is targeted to alter the gene.

### CRISPR Guide RNAs:

In one aspect, the CRISPR/Cas system of the present disclosure further provides a gRNA molecule (e.g., an isolated or non-naturally occurring RNA molecule) that interacts with the RNA-guided nuclease. In certain embodiments, the gRNA is an sgRNA comprising a crRNA sequence comprising a nucleotide sequence which is complementary to a sequence in a target nucleic acid. In some embodiments, the sgRNA further comprises an RNA scaffolding portion (i.e. tracrRNA) that interacts with the RNA-guided nuclease, such that the crRNA is positioned to scan a target nucleic acid for complementarity. In some embodiments, the system is further, optionally, comprised of an oligonucleotide—an HDR template with homology to either side of the target position. See Bloh, K., & Rivera-Torres, N. (2021). International Journal of Molecular Sciences, 22 (8): 3834.

In an embodiment, the RNA-guided nuclease and sgRNA are configured to orient an associated nuclease such that a cleavage event, (e.g., a double strand break or a single strand break) occurs sufficiently close to a complementary sequence in the targeted nucleic acid, thereby facilitating an alteration in the nucleic acid sequence. In some embodiments, the crRNA is 20 nucleotides in length. In some embodiments, the crRNA is 14, 15, 16, 17, 18, 19, 20, 21, 22, 23, 24, 25, 26, 27, 28, 29, or 30 nucleotides in length.

In some embodiments, the crRNA orients the RNA-guided nuclease such that a cleavage event occurs within 1, 2, 3, 4, 5, 10, 15, 20, 25, 30, 35, 40, 45, 50, 55, 60, 65, 70, 75, 80, 85, 90, 95, 100, 125, 150, 175, or 200 nucleotides away from the complementary sequence in the targeted nucleic acid. The double- or single-strand break may be positioned upstream or downstream of the complementary sequence in the targeted nucleic acid. In some embodiments, the cleavage event occurs within a targeted gene. In some embodiments, the cleavage event occurs upstream of a targeted gene.

In certain embodiments, a second gRNA molecule, comprising a second crRNA orients a second RNA-guided nuclease, such that a cleavage event occurs sufficiently close to a complementary sequence in the targeted nucleic acid, thereby facilitating an alteration in the nucleic acid sequence. In some embodiments, the first gRNA and the second gRNA promote a cleavage event within a single targeted gene. In some embodiments, the first gRNA and the second gRNA promote a cleavage event within different targeted genes. In some embodiments, the second crRNA is 20 nucleotides in length. In some embodiments, the second crRNA is 14, 15, 16, 17, 18, 19, 20, 21, 22, 23, 24, 25, 26, 27, 28, 29, or 30 nucleotides in length.

In some embodiments, the second crRNA orients the RNA-guided nuclease such that a cleavage event occurs within 1, 2, 3, 4, 5, 10, 15, 20, 25, 30, 35, 40, 45, 50, 55, 60, 65, 70, 75, 80, 85, 90, 95, 100, 125, 150, 175, or 200 nucleotides away from the complementary sequence in the targeted nucleic acid. The double- or single-strand break may be positioned upstream or downstream of the complementary sequence in the targeted nucleic acid. In some embodiments, the cleavage event occurs within a targeted gene. In some embodiments, the cleavage event occurs upstream of a targeted gene.

In some embodiments, the targeting domains of the first gRNA and the second gRNA are configured such that a cleavage event is positioned, independently for each of the gRNA molecules, within 1, 2, 3, 4, 5, 10, 15, 20, 25, 30, 35, 40, 45, 50, 55, 60, 65, 70, 75, 80, 85, 90, 95, 100, 125, 150, 175, or 200 nucleotides of the others cleavage event. In some embodiments, the first gRNA and the second gRNA molecules alter the targeted nucleic acid sequences simultaneously. In some embodiments, the first gRNA and the second gRNA molecules alter the targeted nucleic acid sequences sequentially.

In some embodiments, a single-strand break is accompanied by a second single-strand break, positioned by the crRNA of a first gRNA and a second gRNA, respectively. For example, the crRNA may orient the associated RNA-guided nucleases such that a cleavage event, (e.g., the two single-strand breaks), are positioned within 1, 2, 3, 4, 5, 10, 15, 20, 25, 30, 35, 40, 45, 50, 55, 60, 65, 70, 75, 80, 85, 90, 95, 100, 125, 150, 175, or 200 nucleotides of one another. In some embodiments, a first crRNA and a second crRNA are configured to orient associated RNA-guided nucleases such that, for example, two single-strand breaks occurs at the same position, or within 1, 2, 3, 4, 5, 10, 15, 20, 25, 30, 35, 40, 45, 50 nucleotides of one another, on opposing strands of genomic DNA, thereby essentially approximating a double strand break.

In some embodiments, the nucleic acid encoding one or more crRNAs is selected from any of SEQ ID NOs: 680-1039. In some embodiments, the nucleic acid encoding one or more crRNAs target hIL1R1 and is selected from any of SEQ ID NOs: 680-824. In some embodiments, the nucleic acid encoding one or more crRNAs target cIL1R1 and is selected from any of SEQ ID NOs: 825-892 and SEQ ID NOs: 3336-3420. In some embodiments, the nucleic acid encoding one or more crRNAs target eIL1R1 and is selected from any of SEQ ID NOs: 893-967. In some embodiments, the nucleic acid encoding one or more crRNAs target fIL1R1 and is selected from any of SEQ ID NOs: 968-1039.

In some embodiments, the nucleic acid encoding one or more crRNAs is selected from any of SEQ ID NOs: 1040-1424. In some embodiments, the nucleic acid encoding one or more crRNAs target hIL1RAP and is selected from any of SEQ ID NOs: 1040-1203. In some embodiments, the nucleic acid encoding one or more crRNAs target cIL1RAP and is selected from any of SEQ ID NOs: 1204-1271 and SEQ ID NOs: 3421-3490. In some embodiments, the nucleic acid encoding one or more crRNAs target eIL1RAP and is selected from any of SEQ ID NOs: 1272-1348. In some embodiments, the nucleic acid encoding one or more crRNAs target fIL1RAP and is selected from any of SEQ ID NOs: 1349-1424.

In some embodiments, the nucleic acid encoding one or more crRNAs is selected from any of SEQ ID NOs: 1425-1546. In some embodiments, the nucleic acid encoding one or more crRNAs target hTGFBR1 and is selected from any of SEQ ID NOs: 1425-1546.

In some embodiments, the nucleic acid encoding one or more crRNAs is selected from any of SEQ ID NOs: 1547-1745. In some embodiments, the nucleic acid encoding one or more crRNAs target hTGFBR2 and is selected from any of SEQ ID NOs: 1547-1745.

In some embodiments, the nucleic acid encoding one or more crRNAs is selected from any of SEQ ID NOs: 1746-1968. In some embodiments, the nucleic acid encoding one or more crRNAs target hIL6R and is selected from any of SEQ ID NOs: 1746-1968.

In some embodiments, the nucleic acid encoding one or more crRNAs is selected from any of SEQ ID NOs: 1969-2178. In some embodiments, the nucleic acid encoding one or more crRNAs target hIL6ST and is selected from any of SEQ ID NOs: 1969-2178.

In some embodiments, the nucleic acid encoding one or more crRNAs is selected from any of SEQ ID NOs: 2179-2395. In some embodiments, the nucleic acid encoding one or more crRNAs target hTNFRSF1A and is selected from any of SEQ ID NOs: 2179-2395.

In some embodiments, the nucleic acid encoding one or more crRNAs is selected from any of SEQ ID NOs: 2396-2642. In some embodiments, the nucleic acid encoding one or more crRNAs target hTNFRSF1B and is selected from any of SEQ ID NOs: 2396-2642.

In some embodiments, the nucleic acid encoding one or more crRNAs is selected from any of SEQ ID NOs: 2643-2866. In some embodiments, the nucleic acid encoding one or more crRNAs target hTNFRSF3 and is selected from any of SEQ ID NOs: 2643-2866.

In some embodiments, the nucleic acid encoding one or more crRNAs is selected from any of SEQ ID NOs: 2867-3041. In some embodiments, the nucleic acid encoding one or more crRNAs target hTNFRSF4 and is selected from any of SEQ ID NOs: 2867-3041.

In some embodiments, the nucleic acid encoding one or more crRNAs is selected from any of SEQ ID NOs: 3042-3335. In some embodiments, the nucleic acid encoding one or more crRNAs target hTNFRSF11A and is selected from any of SEQ ID NOs: 3042-3335.

In some embodiments a nucleic acid encodes a second sgRNA molecule. In some embodiments, a nucleic acid encodes a third sgRNA molecule. In some embodiments, a nucleic acid encodes a fourth sgRNA molecule.

In certain embodiments, a nucleic acid may comprise (a) a sequence encoding a first sgRNA, comprising a crRNA that is complementary with a sequence in a targeted gene, (b) a sequence encoding a second sgRNA, comprising a crRNA that is complementary with a sequence in a second targeted gene, and (c) a sequence encoding an RNA-guided nuclease (e.g., Cas9). Optionally, (d) and (e) are sequences encoding a third sgRNA and a fourth sgRNA, respectively. In some embodiments, the second targeted gene is the same as the first targeted gene. In other embodiments, the second targeted gene is different from the first targeted gene. In some embodiments, (a), (b), and (c) are encoded within the same nucleic acid molecule (e.g., the same vector). In some embodiments, (a) and (b) are encoded within the same nucleic acid molecule. In some embodiments, (a), (b) and (d) are encoded within the same nucleic acid molecule. In some embodiments, (a), (b) and (e) are encoded within the same nucleic acid molecule. In some embodiments, (a), (b), (d) and (e) are encoded within the same nucleic acid molecule. In some embodiments, (a), (b), and (c) are encoded within separate nucleic acid molecules. When more than two sgRNAs are used, any combination of (a), (b), (c), (d) and (e) may be encoded within a single or separate nucleic acid molecules.

In one aspect, the nucleic acid molecules (i.e., those encoding (a), (b), (c), (d) or (e)) are delivered to a target cell (i.e., any combination of the encoded RNA-guided nuclease of (c) and at least one encoded gRNA molecule of (a), (b), (d), or (e) contact a target cell). In some embodiments, said nucleic acid molecules are delivered to a target cell in vivo. In other embodiments, said nucleic acid molecules are delivered to a target cell ex vivo. In some embodiments, said nucleic acid molecules are delivered to a target cell in vitro. In certain embodiments, said nucleic acid molecules are delivered to a target cell as DNA. In other embodiments, said nucleic acid molecules are delivered to a target cell as RNA (e.g., mRNA). In some embodiments, the products of said nucleic acid molecules are delivered as an assembled ribonucleoprotein (RNP).

In some embodiments, contacting a target cell comprises delivering said RNA-guided nuclease of (c), as a protein with at least one said nucleic acid molecules selected from (a), (b), (d), and (e). In some embodiments, contacting a target cell comprises delivering said encoded RNA-guided nuclease of (c), as DNA with at least one said nucleic acid molecules selected from (a), (b), (d), and (e). In some embodiments, contacting a target cell comprises delivering said encoded RNA-guided nuclease of (c), as mRNA with at least one said nucleic acid molecules selected from (a), (b), (d), and (e).

In certain embodiments, CRISPR components are delivered to a target cell via nanoparticles. Exemplary nanoparticles that may be used with all CRISPR/Cas systems disclosed herein include, at least, lipid nanoparticles or liposomes, hydrogel nanoparticles, metalorganic nanoparticles, gold nanoparticles, magnetic nanoparticles and virus-like particles. See generally Xu, C. F. et al. (2021). Advanced Drug Delivery Reviews, 168:3-29.

### B. TALEN

In one aspect, the present disclosure contemplates use of methods, components, and compositions relating to Transcription Activator-Like Effector Nucleases (TALENs) to effectuate augmentation of a ‘nucleic acid sequence (e.g., a targeted gene.

TALE stands for “Transcription Activator-Like Effector” proteins, which include TALENs (“Transcription Activator-Like Effector Nucleases”). A method of using a TALE system for gene editing may also be referred to herein as a TALE method. TALEs are naturally occurring proteins from the plant pathogenic bacteria genus Xanthomonas, and contain DNA-binding domains composed of a series of 33-35-amino-acid repeat domains that each recognizes a single base pair. TALE specificity is determined by two hypervariable amino acids that are known as the repeat-variable di-residues (RVDs). Modular TALE repeats are linked together to recognize contiguous DNA sequences. A specific RVD in the DNA-binding domain recognizes a base in the target locus, providing a structural feature to assemble predictable DNA-binding domains. The DNA binding domains of a TALE are fused to the catalytic domain of a type IIS FokI endonuclease to make a targetable TALE nuclease. To induce site-specific mutation, two individual TALEN arms, separated by a 14-20 base pair spacer region, bring FokI monomers in close proximity to dimerize and produce a targeted double-strand break.

Several large, systematic studies utilizing various assembly methods have indicated that TALE repeats can be combined to recognize virtually any user-defined sequence. Custom-designed TALE arrays are also commercially available through Cellectis Bioresearch (Paris, France), Transposagen Biopharmaceuticals (Lexington, KY, USA), and Life Technologies (Grand Island, NY, USA). TALE and TALEN methods suitable for use in the present disclosure are described in U.S. Patent Application Publication Nos. US 2011/0201118 A1; US 2013/0117869 A1; US 2013/0315884 A1; US 2015/0203871 A1 and US 2016/0120906 A1, the disclosures of which are incorporated by reference herein.

Non-limiting examples of genes that may be silenced or inhibited by permanently gene-editing via a TALE method include IL1R1, IL1RAP, TGFBR1, TGFBR2, IL6R, IL6ST, TNFRSF1A, TNFRSF1B, TNFRSF3, TNFRSF4, TNFRSF11A, and combinations thereof. Non-limiting examples of genes that may be augmented such that their resultant products function as decoys or dominant negatives by permanently gene-editing via a TALE method include IL1R1, IL1RAP, TGFBR1, TGFBR2, IL6R, IL6ST, TNFRSF1A, TNFRSF1B, TNFRSF3, TNFRSF4, TNFRSF11A, and combinations thereof. Non-limiting examples of genes that may be enhanced by permanently gene-editing via a TALE method include IL1R1, IL1RAP, TGFBR1, TGFBR2, IL6R, IL6ST, TNFRSF1A, TNFRSF1B, TNFRSF3, TNFRSF4, TNFRSF11A, and combinations thereof. In an aspect, the disclosure provides compositions for up-regulation of protein receptors (including wildtype or genetically edited), including those that bind to anti-inflammatory cytokines via a TALE method.

Examples of systems, methods, and compositions for altering the expression of a target gene sequence by a TALE method, and which may be used in accordance with embodiments of the present disclosure, are described in U.S. Pat. No. 8,586,526, which is incorporated by reference herein.

### C. Zinc-Finger Nucleases (ZFN)

In one aspect, the present disclosure contemplates use of methods, components, and compositions relating to zinc-finger nucleases (ZFNs) to effectuate augmentation of a ‘nucleic acid sequence (e.g., a targeted gene).

An individual zinc finger contains approximately 30 amino acids in a conserved ββα configuration. Several amino acids on the surface of the α-helix typically contact 3 bp in the major groove of DNA, with varying levels of selectivity. Zinc fingers have two protein domains. The first domain is the DNA binding domain, which includes eukaryotic transcription factors and contain the zinc finger. The second domain is the nuclease domain, which includes the FokI restriction enzyme and is responsible for the catalytic cleavage of DNA.

The DNA-binding domains of individual ZFNs typically contain between three and six individual zinc finger repeats and can each recognize between 9 and 18 base pairs. If the zinc finger domains are specific for their intended target site then even a pair of 3-finger ZFNs that recognize a total of 18 base pairs can, in theory, target a single locus in a mammalian genome. One method to generate new zinc-finger arrays is to combine smaller zinc-finger “modules” of known specificity. The most common modular assembly process involves combining three separate zinc fingers that can each recognize a 3 base pair DNA sequence to generate a 3-finger array that can recognize a 9 base pair target site. Alternatively, selection-based approaches, such as oligomerized pool engineering (OPEN) can be used to select for new zinc-finger arrays from randomized libraries that take into consideration context-dependent interactions between neighboring fingers. Engineered zinc fingers are available commercially; Sangamo Biosciences (Richmond, CA, USA) has developed a propriety platform (CompoZr®) for zinc-finger construction in partnership with Sigma-Aldrich (St. Louis, MO, USA).

Non-limiting examples of genes that may be silenced or inhibited by permanently gene-editing via a zinc finger method include IL1R1, IL1RAP, TGFBR1, TGFBR2, IL6R, IL6ST, TNFRSF1A, TNFRSF1B, TNFRSF3, TNFRSF4, TNFRSF11A, and combinations thereof. Non-limiting examples of genes that may be augmented such that their resultant products function as decoys or dominant negatives by permanently gene-editing via a zinc finger method include IL1R1, IL1RAP, TGFBR1, TGFBR2, IL6R, IL6ST, TNFRSF1A, TNFRSF1B, TNFRSF3, TNFRSF4, TNFRSF11A, and combinations thereof. Non-limiting examples of genes that may be enhanced by permanently gene-editing via a zinc finger method include IL1R1, IL1RAP, TGFBR1, TGFBR2, IL6R, IL6ST, TNFRSF1A, TNFRSF1B, TNFRSF3, TNFRSF4, TNFRSF11A, and combinations thereof. In an aspect, the disclosure provides compositions for up-regulation of protein receptors (including wildtype or genetically edited), including those that bind to anti-inflammatory cytokines via a zinc finger method.

Examples of systems, methods, and compositions for altering the expression of a target gene sequence by a zinc finger method, which may be used in accordance with embodiments of the present disclosure, are described in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, which are incorporated by reference herein.

Other examples of systems, methods, and compositions for altering the expression of a target gene sequence by a zinc finger method, which may be used in accordance with embodiments of the present disclosure, are described in Beane, et al., Mol. Therapy, 2015, 23 1380-1390, the disclosure of which is incorporated by reference herein.

### IV. Joint Disease or Illness

### A. Introduction

As described herein, embodiments of the present disclosure provide compositions and methods for improving joint function and treating joint disease. In particular embodiments, compositions and methods are provided to gene-edit synovial fibroblasts, synoviocytes, chondrocytes, tissue (resident) macrophages, or other cells to reduce pro-inflammatory signaling mediated by the binding of inflammatory cytokines—including, but not limited to, IL1α, IL1β, TNFα, IL6, IL8, IL18, IL33, matrix metalloproteinases (MMPs), TGFβ1, TGFβ2, and combinations thereof—to their cognate receptor(s). Some embodiments are used for treating various forms of arthritis and other inflammatory joint diseases. Some embodiments are further useful for treating canine lameness due to osteoarthritis. Some embodiments are further useful for treating equine lameness due to joint disease. Some embodiments are further useful for treating feline lameness due to joint disease. Some embodiments are also useful for treating post-traumatic arthritis, gout, pseudogout, psoriatic arthritis, and other inflammation-mediated or immune-mediated joint diseases.

Treatment of osteoarthritis, degenerative joint disease, and other joint dysfunctions is complex, and few long-term options exist for either symptomatic relief or restoring joint function. Osteoarthritis (OA) is the leading cause of disability due to pain. See, Neogi, T. (2013). Osteoarthritis Cartilage, 21 (9): 1145-53. OA and similar diseases impact all mammal species, including working animals, domestic pets, and their owners. The common mechanistic thread among joint diseases is the presence of acute of chronic inflammation, which is driven by increased levels of pro-inflammatory cytokine signaling. Joint diseases tend to take a progressive course that encompasses discomfort, pain, and—especially in the case of OA—disability, depending on the degree of disease progression.

Psoriatic arthritis (PsA) is another chronic inflammatory joint disease, in which the joint symptoms are accompanied by skin lesions, such as those commonly associated with psoriasis. See, Boehncke, W. et al. (2014). British Journal of Dermatology, 170 (4): 772-786. Like other forms of arthritis, such as OA, PsA is caused by pro-inflammatory signaling of a host of cytokines, including IL1. Indeed, PsA morbidity has been shown to correlate with single nucleotide polymorphisms (SNPs) that impact the activity of the IL1 gene locus. See, Rahman, P. et al. (2006). Arthritis and Rheumatism, 54 (7): 2321-2325. These studies also implicate inflammatory cytokine signaling, in general, and IL1 more specifically, in disease progression.

Gout is a chronic inflammatory condition that affects joints. The underlying cause is monosodium urate (MSU) crystal deposition and the resultant host response, particularly in joint structures (as well as subcutaneous tissues and other sites). See, Dalbeth, N., & Stamp, L. (2014). Annals of the Rheumatic Diseases, 73 (9): 1598-1600. The clinical manifestations include recurrent acute flares of severe inflammatory arthritis and tendinobursitis. IL1 and other pro-inflammotory mediators are a major contributor to this host response. See, Dinarello, C. A. (2014). Molecular Medicine, 20 (1): S43-S58. To this end, effective blockade of these signaling pathways may provide relief to gout patients.

The current standard of care for many joint disease patients includes anti-inflammatory medications (e.g., NSAIDs) or anti-rheumatics (e.g., methotrexate [inhibitor of AICAR] or adalimumab [anti-TNF alpha monoclonal antibody]). See, Friedman, B., & Cronstein, B. (2019). Joint Bone Spine, 86 (3): 301-307. All of these treatments require repeated dosing for continued effectiveness, which may lead to toxicity issues or tolerance over time. As such, new methods and compositions to treat joint disease and illness are acutely needed to treat these chronic conditions.

In one aspect, the compositions and methods herein described are directed to treat joint disease or illness in a mammal in need thereof. In some embodiments, the joint disease or illness is osteoarthritis. In some embodiments, the joint disease or illness is psoriatic arthritis. In some embodiments, the joint disease or illness is gout.

Among the advantages of the present disclosure over treatments currently available for mammals afflicted with one or more joint disease or illness include the period of relief from symptoms. Upon genetic editing of a cell within a joint, pro-inflammatory signaling is silenced through the targeted gene for the life of that cell and any mitotic progeny. By contrast, biologic treatments require periodic dosing, which may magnify the impact of the host of potentially severe side effects. Among various genetic approaches, the present disclosure is also superior due to, among other reasons, a resistance to leakiness by virtue of modifying a protein receptor, rather than ablating expression of a ligand, which may result in compensatory effects (e.g., buildup of other factors due to lack of negative feedback).

In some embodiments, the present disclosure includes a method for the treatment or prevention of a joint disease or condition in a subject in need thereof, the method comprising administering, to a joint of the subject, a pharmaceutical composition comprising a therapeutically effective amount of a Clustered Regularly Interspaced Short Palindromic Repeats (CRISPR) gene-editing system, the system comprising (i) a CRISPR Associated (Cas) protein; and (ii) at least one guide RNA targeting an IL1R1 gene, IL1RAP gene, TGFBR1 gene, TGFBR2 gene, IL6R gene, IL6ST gene, TNFRSF1A gene, TNFRSF1B gene, TNFRSF3 gene, TNFRSF4 gene, or TNFRSF11A gene or a combination thereof. In some embodiments, the joint disease or condition is osteoarthritis. In some embodiments, the joint disease or condition is psoriatic arthritis. In some embodiments, the joint disease or condition is gout.

In some embodiments, the present disclosure includes a method for the treatment or prevention of an arthritis. Non-limiting examples of arthritis the can be treated using the compositions and methods described herein include post-tramatic arthritis, osteoarthritis (a degenerative condition that affects the joints, most commonly the hips, knees, and hands), rheumatoid arthritis (an autoimmune disorder that causes inflammation in the joints and surrounding tissue), psoriatic arthritis (a type of arthritis that occurs in people with psoriasis, a skin condition characterized by scaly red patches), gout (a type of arthritis caused by the buildup of uric acid crystals in the joints), lupus (a chronic autoimmune disorder that can cause inflammation and damage to the joints, as well as other organs), ankylosing spondylitis (a type of arthritis that primarily affects the spine, causing inflammation and stiffness), reactive arthritis (a type of arthritis that occurs as a reaction to an infection in the body), septic arthritis (a type of arthritis caused by an infection in the joint), juvenile idiopathic arthritis (a form of arthritis that affects children under the age of 16), and fibromyalgia (a chronic pain disorder that can cause widespread pain and stiffness, including in the joints).

In some embodiments, the present disclosure includes a method for the treatment or prevention of pseudogout, Crystal arthropathies (caused by the formation of crystals in the joints, such as gout and pseudogout), or CPPD disease (calcium pyrophosphate deposition disease) also called chondroclacinosis.

In some embodiments, the present disclosure includes a method for the treatment or prevention of rheumatoid arthritis, psoriasis, asthma, inflammatory bowel disease, multiple sclerosis, Alzheimer's disease, Type 2 diabetes, cardiovascular disease, or cancer. In some embodiments, these disorders are treated by administering a CRISPR composition, as described herein, targeting an IL1 receptor, e.g., IL1R1 or IL1AP.

### B. Osteoarthritis

In one aspect, the present disclosure encompasses treatments for osteoarthritis (OA). In some embodiments, OA treatment comprises a therapeutically effective amount of a Clustered Regularly Interspaced Short Palindromic Repeats (CRISPR) gene-editing system, the system comprising: (i) a CRISPR Associated (Cas) protein; and (ii) at least one guide RNA targeting IL1R1. In some embodiments, the OA treatment comprises a CRISPR gene-editing system targeting hIL1R1. In some embodiments, the OA treatment comprises a CRISPR gene-editing system targeting cIL1R1. In some embodiments, the OA treatment comprises a CRISP gene-editing system R targeting eIL1R1. In some embodiments, the OA treatment comprises a CRISPR gene-editing system targeting fIL1R1.

In some embodiments, the CRISPR gene-editing system for the treatment of OA comprises one or more sgRNAs targeting exon 1 of hIL1R1. In some embodiments, the CRISPR gene-editing system for the treatment of OA comprises one or more sgRNAs targeting exon 2 of hIL1R1. In some embodiments, the CRISPR gene-editing system for the treatment of OA comprises one or more sgRNAs targeting exon 3 of hIL1R1. In some embodiments, the CRISPR gene-editing system for the treatment of OA comprises one or more sgRNAs targeting exon 4 of hIL1R1. In some embodiments, the CRISPR gene-editing system for the treatment of OA comprises one or more sgRNAs targeting exon 5 of hIL1R1. In some embodiments, the CRISPR gene-editing system for the treatment of OA comprises one or more sgRNAs targeting exon 6 of hIL1R1. In some embodiments, the CRISPR gene-editing system for the treatment of OA comprises one or more sgRNAs targeting exon 7 of hIL1R1. In some embodiments, the CRISPR gene-editing system for the treatment of OA comprises one or more sgRNAs targeting exon 8 of hIL1R1. In some embodiments, the CRISPR gene-editing system for the treatment of OA comprises one or more sgRNAs targeting exon 9 of hIL1R1. In some embodiments, the CRISPR gene-editing system for the treatment of OA comprises one or more sgRNAs targeting exon 10 of hIL1R1. In some embodiments, the CRISPR gene-editing system for the treatment of OA comprises one or more sgRNAs targeting exon 11 of hIL1R1. In some embodiments, the CRISPR gene-editing system for the treatment of OA comprises one or more sgRNAs targeting exon 12 of hIL1R1. In some embodiments, the CRISPR gene-editing system for the treatment of OA comprises one or more sgRNAs targeting exon 13 of hIL1R1. In some embodiments, the CRISPR gene-editing system for the treatment of OA comprises one or more sgRNAs targeting exon 14 of hIL1R1. In some embodiments, the CRISPR gene-editing system for the treatment of OA comprises one or more sgRNAs targeting exon 15 of hIL1R1. In some embodiments, the CRISPR gene-editing system for the treatment of OA comprises one or more sgRNAs targeting exon 16 of hIL1R1. In some embodiments, the CRISPR gene-editing system for the treatment of OA comprises one or more sgRNAs targeting exon 17 of hIL1R1. In some embodiments, the CRISPR gene-editing system for the treatment of OA comprises one or more sgRNAs targeting exon 18 of hIL1R1. In some embodiments, the CRISPR gene-editing system for the treatment of OA comprises one or more sgRNAs targeting exon 19 of hIL1R1. In some embodiments, the CRISPR gene-editing system for the treatment of OA comprises one or more sgRNAs targeting exon 20 of hIL1R1. In some embodiments, the CRISPR gene-editing system for the treatment of OA comprises one or more sgRNAs targeting exon 21 of hIL1R1.

In some embodiments, the CRISPR gene-editing system for the treatment of OA comprises one or more sgRNAs targeting exon 1 of cIL1R1. In some embodiments, the CRISPR gene-editing system for the treatment of OA comprises one or more sgRNAs targeting exon 2 of cIL1R1. In some embodiments, the CRISPR gene-editing system for the treatment of OA comprises one or more sgRNAs targeting exon 3 of cIL1R1. In some embodiments, the CRISPR gene-editing system for the treatment of OA comprises one or more sgRNAs targeting exon 4 of cIL1R1. In some embodiments, the CRISPR gene-editing system for the treatment of OA comprises one or more sgRNAs targeting exon 5 of cIL1R1. In some embodiments, the CRISPR gene-editing system for the treatment of OA comprises one or more sgRNAs targeting exon 6 of cIL1R1. In some embodiments, the CRISPR gene-editing system for the treatment of OA comprises one or more sgRNAs targeting exon 7 of cIL1R1. In some embodiments, the CRISPR gene-editing system for the treatment of OA comprises one or more sgRNAs targeting exon 8 of cIL1R1. In some embodiments, the CRISPR gene-editing system for the treatment of OA comprises one or more sgRNAs targeting exon 9 of cIL1R1. In some embodiments, the CRISPR gene-editing system for the treatment of OA comprises one or more sgRNAs targeting exon 10 of cIL1R1. In some embodiments, the CRISPR gene-editing system for the treatment of OA comprises one or more sgRNAs targeting exon 11 of cIL1R1. In some embodiments, the CRISPR gene-editing system for the treatment of OA comprises one or more sgRNAs targeting exon 12 of cIL1R1. In some embodiments, the CRISPR gene-editing system for the treatment of OA comprises one or more sgRNAs targeting exon 13 of cIL1R1. In some embodiments, the CRISPR gene-editing system for the treatment of OA comprises one or more sgRNAs targeting exon 14 of cIL1R1. In some embodiments, the CRISPR gene-editing system for the treatment of OA comprises one or more sgRNAs targeting exon 15 of cIL1R1. In some embodiments, the CRISPR gene-editing system for the treatment of OA comprises one or more sgRNAs targeting exon 16 of cIL1R1.

In some embodiments, the CRISPR gene-editing system for the treatment of OA comprises one or more sgRNAs targeting exon 1 of eIL1R1. In some embodiments, the CRISPR gene-editing system for the treatment of OA comprises one or more sgRNAs targeting exon 2 of eIL1R1. In some embodiments, the CRISPR gene-editing system for the treatment of OA comprises one or more sgRNAs targeting exon 3 of eIL1R1. In some embodiments, the CRISPR gene-editing system for the treatment of OA comprises one or more sgRNAs targeting exon 4 of eIL1R1. In some embodiments, the CRISPR gene-editing system for the treatment of OA comprises one or more sgRNAs targeting exon 5 of eIL1R1. In some embodiments, the CRISPR gene-editing system for the treatment of OA comprises one or more sgRNAs targeting exon 6 of eIL1R1. In some embodiments, the CRISPR gene-editing system for the treatment of OA comprises one or more sgRNAs targeting exon 7 of eIL1R1. In some embodiments, the CRISPR gene-editing system for the treatment of OA comprises one or more sgRNAs targeting exon 8 of eIL1R1. In some embodiments, the CRISPR gene-editing system for the treatment of OA comprises one or more sgRNAs targeting exon 9 of eIL1R1. In some embodiments, the CRISPR gene-editing system for the treatment of OA comprises one or more sgRNAs targeting exon 10 of eIL1R1. In some embodiments, the CRISPR gene-editing system for the treatment of OA comprises one or more sgRNAs targeting exon 11 of eIL1R1. In some embodiments, the CRISPR gene-editing system for the treatment of OA comprises one or more sgRNAs targeting exon 12 of eIL1R1. In some embodiments, the CRISPR gene-editing system for the treatment of OA comprises one or more sgRNAs targeting exon 13 of eIL1R1. In some embodiments, the CRISPR gene-editing system for the treatment of OA comprises one or more sgRNAs targeting exon 14 of eIL1R1. In some embodiments, the CRISPR gene-editing system for the treatment of OA comprises one or more sgRNAs targeting exon 15 of eIL1R1.

In some embodiments, the CRISPR gene-editing system for the treatment of OA comprises one or more sgRNAs targeting exon 1 of fIL1R1. In some embodiments, the CRISPR gene-editing system for the treatment of OA comprises one or more sgRNAs targeting exon 2 of fIL1R1. In some embodiments, the CRISPR gene-editing system for the treatment of OA comprises one or more sgRNAs targeting exon 3 of fIL1R1. In some embodiments, the CRISPR gene-editing system for the treatment of OA comprises one or more sgRNAs targeting exon 4 of fIL1R1. In some embodiments, the CRISPR gene-editing system for the treatment of OA comprises one or more sgRNAs targeting exon 5 of fIL1R1. In some embodiments, the CRISPR gene-editing system for the treatment of OA comprises one or more sgRNAs targeting exon 6 of fIL1R1. In some embodiments, the CRISPR gene-editing system for the treatment of OA comprises one or more sgRNAs targeting exon 7 of fIL1R1. In some embodiments, the CRISPR gene-editing system for the treatment of OA comprises one or more sgRNAs targeting exon 8 of fIL1R1. In some embodiments, the CRISPR gene-editing system for the treatment of OA comprises one or more sgRNAs targeting exon 9 of fIL1R1. In some embodiments, the CRISPR gene-editing system for the treatment of OA comprises one or more sgRNAs targeting exon 10 of fIL1R1. In some embodiments, the CRISPR gene-editing system for the treatment of OA comprises one or more sgRNAs targeting exon 11 of fIL1R1. In some embodiments, the CRISPR gene-editing system for the treatment of OA comprises one or more sgRNAs targeting exon 12 of fIL1R1. In some embodiments, the CRISPR gene-editing system for the treatment of OA comprises one or more sgRNAs targeting exon 13 of fIL1R1. In some embodiments, the CRISPR gene-editing system for the treatment of OA comprises one or more sgRNAs targeting exon 14 of fIL1R1. In some embodiments, the CRISPR gene-editing system for the treatment of OA comprises one or more sgRNAs targeting exon 15 of fIL1R1. In some embodiments, the CRISPR gene-editing system for the treatment of OA comprises one or more sgRNAs targeting exon 16 of fIL1R1. In some embodiments, the CRISPR gene-editing system for the treatment of OA comprises one or more sgRNAs targeting exon 17 of fIL1R1. In some embodiments, the CRISPR gene-editing system for the treatment of OA comprises one or more sgRNAs targeting exon 18 of fIL1R1.

In some embodiments, the CRISPR gene-editing system for the treatment of OA comprises one or more sgRNAs targeting IL1RAP. In some embodiments, the CRISPR gene-editing system for the treatment of OA comprises one or more sgRNAs targeting hIL1RAP. In some embodiments, the CRISPR gene-editing system for the treatment of OA comprises one or more sgRNAs targeting cIL1RAP. In some embodiments, the CRISPR gene-editing system for the treatment of OA comprises one or more sgRNAs targeting eIL1RAP. In some embodiments, the CRISPR gene-editing system for the treatment of OA comprises one or more sgRNAs targeting fIL1RAP.

In some embodiments, the CRISPR gene-editing system for the treatment of OA comprises one or more sgRNAs targeting exon 1 of hIL1RAP. In some embodiments, the CRISPR gene-editing system for the treatment of OA comprises one or more sgRNAs targeting exon 2 of hIL1RAP. In some embodiments, the CRISPR gene-editing system for the treatment of OA comprises one or more sgRNAs targeting exon 3 of hIL1RAP. In some embodiments, the CRISPR gene-editing system for the treatment of OA comprises one or more sgRNAs targeting exon 4 of hIL1RAP. In some embodiments, the CRISPR gene-editing system for the treatment of OA comprises one or more sgRNAs targeting exon 5 of hIL1RAP. In some embodiments, the CRISPR gene-editing system for the treatment of OA comprises one or more sgRNAs targeting exon 6 of hIL1RAP. In some embodiments, the CRISPR gene-editing system for the treatment of OA comprises one or more sgRNAs targeting exon 7 of hIL1RAP. In some embodiments, the CRISPR gene-editing system for the treatment of OA comprises one or more sgRNAs targeting exon 8 of hIL1RAP. In some embodiments, the CRISPR gene-editing system for the treatment of OA comprises one or more sgRNAs targeting exon 9 of hIL1RAP. In some embodiments, the CRISPR gene-editing system for the treatment of OA comprises one or more sgRNAs targeting exon 10 of hIL1RAP. In some embodiments, the CRISPR gene-editing system for the treatment of OA comprises one or more sgRNAs targeting exon 11 of hIL1RAP. In some embodiments, the CRISPR gene-editing system for the treatment of OA comprises one or more sgRNAs targeting exon 12 of hIL1RAP. In some embodiments, the CRISPR gene-editing system for the treatment of OA comprises one or more sgRNAs targeting exon 13 of hIL1RAP. In some embodiments, the CRISPR gene-editing system for the treatment of OA comprises one or more sgRNAs targeting exon 14 of hIL1RAP. In some embodiments, the CRISPR gene-editing system for the treatment of OA comprises one or more sgRNAs targeting exon 15 of hIL1RAP. In some embodiments, the CRISPR gene-editing system for the treatment of OA comprises one or more sgRNAs targeting exon 16 of hIL1RAP. In some embodiments, the CRISPR gene-editing system for the treatment of OA comprises one or more sgRNAs targeting exon 17 of hIL1RAP.

In some embodiments, the CRISPR gene-editing system for the treatment of OA comprises one or more sgRNAs targeting exon 1 of cIL1RAP. In some embodiments, the CRISPR gene-editing system for the treatment of OA comprises one or more sgRNAs targeting exon 2 of cIL1RAP. In some embodiments, the CRISPR gene-editing system for the treatment of OA comprises one or more sgRNAs targeting exon 3 of cIL1RAP. In some embodiments, the CRISPR gene-editing system for the treatment of OA comprises one or more sgRNAs targeting exon 4 of cIL1RAP. In some embodiments, the CRISPR gene-editing system for the treatment of OA comprises one or more sgRNAs targeting exon 5 of cIL1RAP. In some embodiments, the CRISPR gene-editing system for the treatment of OA comprises one or more sgRNAs targeting exon 6 of cIL1RAP. In some embodiments, the CRISPR gene-editing system for the treatment of OA comprises one or more sgRNAs targeting exon 7 of cIL1RAP. In some embodiments, the CRISPR gene-editing system for the treatment of OA comprises one or more sgRNAs targeting exon 8 of cIL1RAP. In some embodiments, the CRISPR gene-editing system for the treatment of OA comprises one or more sgRNAs targeting exon 9 of cIL1RAP. In some embodiments, the CRISPR gene-editing system for the treatment of OA comprises one or more sgRNAs targeting exon 10 of cIL1RAP. In some embodiments, the CRISPR gene-editing system for the treatment of OA comprises one or more sgRNAs targeting exon 11 of cIL1RAP. In some embodiments, the CRISPR gene-editing system for the treatment of OA comprises one or more sgRNAs targeting exon 12 of cIL1RAP. In some embodiments, the CRISPR gene-editing system for the treatment of OA comprises one or more sgRNAs targeting exon 13 of cIL1RAP. In some embodiments, the CRISPR gene-editing system for the treatment of OA comprises one or more sgRNAs targeting exon 14 of cIL1RAP. In some embodiments, the CRISPR gene-editing system for the treatment of OA comprises one or more sgRNAs targeting exon 15 of cIL1RAP. In some embodiments, the CRISPR gene-editing system for the treatment of OA comprises one or more sgRNAs targeting exon 16 of cIL1RAP.

In some embodiments, the CRISPR gene-editing system for the treatment of OA comprises one or more sgRNAs targeting exon 1 of eIL1RAP. In some embodiments, the CRISPR gene-editing system for the treatment of OA comprises one or more sgRNAs targeting exon 2 of eIL1RAP. In some embodiments, the CRISPR gene-editing system for the treatment of OA comprises one or more sgRNAs targeting exon 3 of eIL1RAP. In some embodiments, the CRISPR gene-editing system for the treatment of OA comprises one or more sgRNAs targeting exon 4 of eIL1RAP. In some embodiments, the CRISPR gene-editing system for the treatment of OA comprises one or more sgRNAs targeting exon 5 of eIL1RAP. In some embodiments, the CRISPR gene-editing system for the treatment of OA comprises one or more sgRNAs targeting exon 6 of eIL1RAP. In some embodiments, the CRISPR gene-editing system for the treatment of OA comprises one or more sgRNAs targeting exon 7 of eIL1RAP. In some embodiments, the CRISPR gene-editing system for the treatment of OA comprises one or more sgRNAs targeting exon 8 of eIL1RAP. In some embodiments, the CRISPR gene-editing system for the treatment of OA comprises one or more sgRNAs targeting exon 9 of eIL1RAP. In some embodiments, the CRISPR gene-editing system for the treatment of OA comprises one or more sgRNAs targeting exon 10 of eIL1RAP. In some embodiments, the CRISPR gene-editing system for the treatment of OA comprises one or more sgRNAs targeting exon 11 of eIL1RAP. In some embodiments, the CRISPR gene-editing system for the treatment of OA comprises one or more sgRNAs targeting exon 12 of eIL1RAP. In some embodiments, the CRISPR gene-editing system for the treatment of OA comprises one or more sgRNAs targeting exon 13 of eIL1RAP.

In some embodiments, the CRISPR gene-editing system for the treatment of OA comprises one or more sgRNAs targeting exon 1 of fIL1RAP. In some embodiments, the CRISPR gene-editing system for the treatment of OA comprises one or more sgRNAs targeting exon 2 of fIL1RAP. In some embodiments, the CRISPR gene-editing system for the treatment of OA comprises one or more sgRNAs targeting exon 3 of fIL1RAP. In some embodiments, the CRISPR gene-editing system for the treatment of OA comprises one or more sgRNAs targeting exon 4 of fIL1RAP. In some embodiments, the CRISPR gene-editing system for the treatment of OA comprises one or more sgRNAs targeting exon 5 of fIL1RAP. In some embodiments, the CRISPR gene-editing system for the treatment of OA comprises one or more sgRNAs targeting exon 6 of fIL1RAP. In some embodiments, the CRISPR gene-editing system for the treatment of OA comprises one or more sgRNAs targeting exon 7 of fIL1RAP. In some embodiments, the CRISPR gene-editing system for the treatment of OA comprises one or more sgRNAs targeting exon 8 of fIL1RAP. In some embodiments, the CRISPR gene-editing system for the treatment of OA comprises one or more sgRNAs targeting exon 9 of fIL1RAP. In some embodiments, the CRISPR gene-editing system for the treatment of OA comprises one or more sgRNAs targeting exon 10 of fIL1RAP. In some embodiments, the CRISPR gene-editing system for the treatment of OA comprises one or more sgRNAs targeting exon 11 of fIL1RAP. In some embodiments, the CRISPR gene-editing system for the treatment of OA comprises one or more sgRNAs targeting exon 12 of fIL1RAP. In some embodiments, the CRISPR gene-editing system for the treatment of OA comprises one or more sgRNAs targeting exon 13 of fIL1RAP. In some embodiments, the CRISPR gene-editing system for the treatment of OA comprises one or more sgRNAs targeting exon 14 of fIL1RAP.

In some embodiments, the CRISPR gene-editing system for the treatment of OA comprises one or more sgRNAs targeting TGFBR1. In some embodiments, the CRISPR gene-editing system for the treatment of OA comprises one or more sgRNAs targeting hTGFBR1. In some embodiments, the CRISPR gene-editing system for the treatment of OA comprises one or more sgRNAs targeting cTGFBR1. In some embodiments, the CRISPR gene-editing system for the treatment of OA comprises one or more sgRNAs targeting e TGFBR1. In some embodiments, the CRISPR gene-editing system for the treatment of OA comprises one or more sgRNAs targeting fTGFBR1.

In some embodiments, the CRISPR gene-editing system for the treatment of OA comprises one or more sgRNAs targeting exon 1 of hTGFBR1. In some embodiments, the CRISPR gene-editing system for the treatment of OA comprises one or more sgRNAs targeting exon 2 of hTGFBR1. In some embodiments, the CRISPR gene-editing system for the treatment of OA comprises one or more sgRNAs targeting exon 3 of hTGFBR1. In some embodiments, the CRISPR gene-editing system for the treatment of OA comprises one or more sgRNAs targeting exon 4 of hTGFBR1. In some embodiments, the CRISPR gene-editing system for the treatment of OA comprises one or more sgRNAs targeting exon 5 of hTGFBR1. In some embodiments, the CRISPR gene-editing system for the treatment of OA comprises one or more sgRNAs targeting exon 6 of hTGFBR1. In some embodiments, the CRISPR gene-editing system for the treatment of OA comprises one or more sgRNAs targeting exon 7 of hTGFBR1. In some embodiments, the CRISPR gene-editing system for the treatment of OA comprises one or more sgRNAs targeting exon 8 of hTGFBR1. In some embodiments, the CRISPR gene-editing system for the treatment of OA comprises one or more sgRNAs targeting exon 9 of hTGFBR1. In some embodiments, the CRISPR gene-editing system for the treatment of OA comprises one or more sgRNAs targeting exon 10 of hTGFBR1. In some embodiments, the CRISPR gene-editing system for the treatment of OA comprises one or more sgRNAs targeting exon 11 of hTGFBR1.

In some embodiments, the CRISPR gene-editing system for the treatment of OA comprises one or more sgRNAs targeting exon 1 of cTGFBR1. In some embodiments, the CRISPR gene-editing system for the treatment of OA comprises one or more sgRNAs targeting exon 2 of cTGFBR1. In some embodiments, the CRISPR gene-editing system for the treatment of OA comprises one or more sgRNAs targeting exon 3 of cTGFBR1. In some embodiments, the CRISPR gene-editing system for the treatment of OA comprises one or more sgRNAs targeting exon 4 of cTGFBR1. In some embodiments, the CRISPR gene-editing system for the treatment of OA comprises one or more sgRNAs targeting exon 5 of cTGFBR1. In some embodiments, the CRISPR gene-editing system for the treatment of OA comprises one or more sgRNAs targeting exon 6 of cTGFBR1. In some embodiments, the CRISPR gene-editing system for the treatment of OA comprises one or more sgRNAs targeting exon 7 of cTGFBR1. In some embodiments, the CRISPR gene-editing system for the treatment of OA comprises one or more sgRNAs targeting exon 8 of cTGFBR1. In some embodiments, the CRISPR gene-editing system for the treatment of OA comprises one or more sgRNAs targeting exon 9 of cTGFBR1.

In some embodiments, the CRISPR gene-editing system for the treatment of OA comprises one or more sgRNAs targeting exon 1 of eTGFBR1. In some embodiments, the CRISPR gene-editing system for the treatment of OA comprises one or more sgRNAs targeting exon 2 of eTGFBR1. In some embodiments, the CRISPR gene-editing system for the treatment of OA comprises one or more sgRNAs targeting exon 3 of eTGFBR1. In some embodiments, the CRISPR gene-editing system for the treatment of OA comprises one or more sgRNAs targeting exon 4 of eTGFBR1. In some embodiments, the CRISPR gene-editing system for the treatment of OA comprises one or more sgRNAs targeting exon 5 of eTGFBR1. In some embodiments, the CRISPR gene-editing system for the treatment of OA comprises one or more sgRNAs targeting exon 6 of eTGFBR1. In some embodiments, the CRISPR gene-editing system for the treatment of OA comprises one or more sgRNAs targeting exon 7 of eTGFBR1. In some embodiments, the CRISPR gene-editing system for the treatment of OA comprises one or more sgRNAs targeting exon 8 of eTGFBR1. In some embodiments, the CRISPR gene-editing system for the treatment of OA comprises one or more sgRNAs targeting exon 9 of eTGFBR1. In some embodiments, the CRISPR gene-editing system for the treatment of OA comprises one or more sgRNAs targeting exon 10 of eTGFBR1. In some embodiments, the CRISPR gene-editing system for the treatment of OA comprises one or more sgRNAs targeting exon 11 of eTGFBR1. In some embodiments, the CRISPR gene-editing system for the treatment of OA comprises one or more sgRNAs targeting exon 12 of eTGFBR1.

In some embodiments, the CRISPR gene-editing system for the treatment of OA comprises one or more sgRNAs targeting exon 1 of fTGFBR1. In some embodiments, the CRISPR gene-editing system for the treatment of OA comprises one or more sgRNAs targeting exon 2 of fTGFBR1. In some embodiments, the CRISPR gene-editing system for the treatment of OA comprises one or more sgRNAs targeting exon 3 of fTGFBR1. In some embodiments, the CRISPR gene-editing system for the treatment of OA comprises one or more sgRNAs targeting exon 4 of fTGFBR1. In some embodiments, the CRISPR gene-editing system for the treatment of OA comprises one or more sgRNAs targeting exon 5 of fTGFBR1. In some embodiments, the CRISPR gene-editing system for the treatment of OA comprises one or more sgRNAs targeting exon 6 of fTGFBR1. In some embodiments, the CRISPR gene-editing system for the treatment of OA comprises one or more sgRNAs targeting exon 7 of fTGFBR1. In some embodiments, the CRISPR gene-editing system for the treatment of OA comprises one or more sgRNAs targeting exon 8 of fTGFBR1. In some embodiments, the CRISPR gene-editing system for the treatment of OA comprises one or more sgRNAs targeting exon 9 of fTGFBR1. In some embodiments, the CRISPR gene-editing system for the treatment of OA comprises one or more sgRNAs targeting exon 10 of fTGFBR1. In some embodiments, the CRISPR gene-editing system for the treatment of OA comprises one or more sgRNAs targeting exon 11 of fTGFBR1.

In some embodiments, the CRISPR gene-editing system for the treatment of OA comprises one or more sgRNAs targeting TGFBR2. In some embodiments, the CRISPR gene-editing system for the treatment of OA comprises one or more sgRNAs targeting hTGFBR2. In some embodiments, the CRISPR gene-editing system for the treatment of OA comprises one or more sgRNAs targeting cTGFBR2. In some embodiments, the CRISPR gene-editing system for the treatment of OA comprises one or more sgRNAs targeting eTGFBR2. In some embodiments, the CRISPR gene-editing system for the treatment of OA comprises one or more sgRNAs targeting fTGFBR2.

In some embodiments, the CRISPR gene-editing system for the treatment of OA comprises one or more sgRNAs targeting exon 1 of hTGFBR2. In some embodiments, the CRISPR gene-editing system for the treatment of OA comprises one or more sgRNAs targeting exon 2 of hTGFBR2. In some embodiments, the CRISPR gene-editing system for the treatment of OA comprises one or more sgRNAs targeting exon 3 of hTGFBR2. In some embodiments, the CRISPR gene-editing system for the treatment of OA comprises one or more sgRNAs targeting exon 4 of hTGFBR2. In some embodiments, the CRISPR gene-editing system for the treatment of OA comprises one or more sgRNAs targeting exon 5 of hTGFBR2. In some embodiments, the CRISPR gene-editing system for the treatment of OA comprises one or more sgRNAs targeting exon 6 of hTGFBR2. In some embodiments, the CRISPR gene-editing system for the treatment of OA comprises one or more sgRNAs targeting exon 7 of hTGFBR2. In some embodiments, the CRISPR gene-editing system for the treatment of OA comprises one or more sgRNAs targeting exon 8 of hTGFBR2. In some embodiments, the CRISPR gene-editing system for the treatment of OA comprises one or more sgRNAs targeting exon 9 of hTGFBR2.

In some embodiments, the CRISPR gene-editing system for the treatment of OA comprises one or more sgRNAs targeting exon 1 of cTGFBR2. In some embodiments, the CRISPR gene-editing system for the treatment of OA comprises one or more sgRNAs targeting exon 2 of cTGFBR2. In some embodiments, the CRISPR gene-editing system for the treatment of OA comprises one or more sgRNAs targeting exon 3 of cTGFBR2. In some embodiments, the CRISPR gene-editing system for the treatment of OA comprises one or more sgRNAs targeting exon 4 of cTGFBR2. In some embodiments, the CRISPR gene-editing system for the treatment of OA comprises one or more sgRNAs targeting exon 5 of cTGFBR2. In some embodiments, the CRISPR gene-editing system for the treatment of OA comprises one or more sgRNAs targeting exon 6 of cTGFBR2. In some embodiments, the CRISPR gene-editing system for the treatment of OA comprises one or more sgRNAs targeting exon 7 of cTGFBR2. In some embodiments, the CRISPR gene-editing system for the treatment of OA comprises one or more sgRNAs targeting exon 8 of cTGFBR2.

In some embodiments, the CRISPR gene-editing system for the treatment of OA comprises one or more sgRNAs targeting exon 1 of eTGFBR2. In some embodiments, the CRISPR gene-editing system for the treatment of OA comprises one or more sgRNAs targeting exon 2 of eTGFBR2. In some embodiments, the CRISPR gene-editing system for the treatment of OA comprises one or more sgRNAs targeting exon 3 of eTGFBR2. In some embodiments, the CRISPR gene-editing system for the treatment of OA comprises one or more sgRNAs targeting exon 4 of eTGFBR2. In some embodiments, the CRISPR gene-editing system for the treatment of OA comprises one or more sgRNAs targeting exon 5 of eTGFBR2. In some embodiments, the CRISPR gene-editing system for the treatment of OA comprises one or more sgRNAs targeting exon 6 of eTGFBR2. In some embodiments, the CRISPR gene-editing system for the treatment of OA comprises one or more sgRNAs targeting exon 7 of eTGFBR2. In some embodiments, the CRISPR gene-editing system for the treatment of OA comprises one or more sgRNAs targeting exon 8 of eTGFBR2. In some embodiments, the CRISPR gene-editing system for the treatment of OA comprises one or more sgRNAs targeting exon 9 of eTGFBR2. In some embodiments, the CRISPR gene-editing system for the treatment of OA comprises one or more sgRNAs targeting exon 10 of eTGFBR2.

In some embodiments, the CRISPR gene-editing system for the treatment of OA comprises one or more sgRNAs targeting exon 1 of fTGFBR2. In some embodiments, the CRISPR gene-editing system for the treatment of OA comprises one or more sgRNAs targeting exon 2 of fTGFBR2. In some embodiments, the CRISPR gene-editing system for the treatment of OA comprises one or more sgRNAs targeting exon 3 of fTGFBR2. In some embodiments, the CRISPR gene-editing system for the treatment of OA comprises one or more sgRNAs targeting exon 4 of fTGFBR2. In some embodiments, the CRISPR gene-editing system for the treatment of OA comprises one or more sgRNAs targeting exon 5 of fTGFBR2. In some embodiments, the CRISPR gene-editing system for the treatment of OA comprises one or more sgRNAs targeting exon 6 of fTGFBR2. In some embodiments, the CRISPR gene-editing system for the treatment of OA comprises one or more sgRNAs targeting exon 7 of fTGFBR2. In some embodiments, the CRISPR gene-editing system for the treatment of OA comprises one or more sgRNAs targeting exon 8 of fTGFBR2.

In some embodiments, the CRISPR gene-editing system for the treatment of OA comprises one or more sgRNAs targeting IL6R. In some embodiments, the CRISPR gene-editing system for the treatment of OA comprises one or more sgRNAs targeting hIL6R. In some embodiments, the CRISPR gene-editing system for the treatment of OA comprises one or more sgRNAs targeting cIL6R. In some embodiments, the CRISPR gene-editing system for the treatment of OA comprises one or more sgRNAs targeting eIL6R. In some embodiments, the CRISPR gene-editing system for the treatment of OA comprises one or more sgRNAs targeting fIL6R.

In some embodiments, the CRISPR gene-editing system for the treatment of OA comprises one or more sgRNAs targeting exon 1 of hIL6R. In some embodiments, the CRISPR gene-editing system for the treatment of OA comprises one or more sgRNAs targeting exon 2 of hIL6R. In some embodiments, the CRISPR gene-editing system for the treatment of OA comprises one or more sgRNAs targeting exon 3 of hIL6R. In some embodiments, the CRISPR gene-editing system for the treatment of OA comprises one or more sgRNAs targeting exon 4 of hIL6R. In some embodiments, the CRISPR gene-editing system for the treatment of OA comprises one or more sgRNAs targeting exon 5 of hIL6R. In some embodiments, the CRISPR gene-editing system for the treatment of OA comprises one or more sgRNAs targeting exon 6 of hIL6R. In some embodiments, the CRISPR gene-editing system for the treatment of OA comprises one or more sgRNAs targeting exon 7 of hIL6R. In some embodiments, the CRISPR gene-editing system for the treatment of OA comprises one or more sgRNAs targeting exon 8 of hIL6R. In some embodiments, the CRISPR gene-editing system for the treatment of OA comprises one or more sgRNAs targeting exon 9 of hIL6R. In some embodiments, the CRISPR gene-editing system for the treatment of OA comprises one or more sgRNAs targeting exon 10 of hIL6R. In some embodiments, the CRISPR gene-editing system for the treatment of OA comprises one or more sgRNAs targeting exon 11 of hIL6R In some embodiments, the CRISPR gene-editing system for the treatment of OA comprises one or more sgRNAs targeting exon 12 of hIL6R. In some embodiments, the CRISPR gene-editing system for the treatment of OA comprises one or more sgRNAs targeting exon 13 of hIL6R. In some embodiments, the CRISPR gene-editing system for the treatment of OA comprises one or more sgRNAs targeting exon 14 of hIL6R. In some embodiments, the CRISPR gene-editing system for the treatment of OA comprises one or more sgRNAs targeting exon 15 of hIL6R.

In some embodiments, the CRISPR gene-editing system for the treatment of OA comprises one or more sgRNAs targeting exon 1 of cIL6R. In some embodiments, the CRISPR gene-editing system for the treatment of OA comprises one or more sgRNAs targeting exon 2 of cIL6R. In some embodiments, the CRISPR gene-editing system for the treatment of OA comprises one or more sgRNAs targeting exon 3 of cIL6R. In some embodiments, the CRISPR gene-editing system for the treatment of OA comprises one or more sgRNAs targeting exon 4 of cIL6R. In some embodiments, the CRISPR gene-editing system for the treatment of OA comprises one or more sgRNAs targeting exon 5 of cIL6R. In some embodiments, the CRISPR gene-editing system for the treatment of OA comprises one or more sgRNAs targeting exon 6 of cIL6R. In some embodiments, the CRISPR gene-editing system for the treatment of OA comprises one or more sgRNAs targeting exon 7 of cIL6R. In some embodiments, the CRISPR gene-editing system for the treatment of OA comprises one or more sgRNAs targeting exon 8 of cIL6R. In some embodiments, the CRISPR gene-editing system for the treatment of OA comprises one or more sgRNAs targeting exon 9 of cIL6R. In some embodiments, the CRISPR gene-editing system for the treatment of OA comprises one or more sgRNAs targeting exon 10 of cIL6R. In some embodiments, the CRISPR gene-editing system for the treatment of OA comprises one or more sgRNAs targeting exon 11 of cIL6R. In some embodiments, the CRISPR gene-editing system for the treatment of OA comprises one or more sgRNAs targeting exon 12 of cIL6R. In some embodiments, the CRISPR gene-editing system for the treatment of OA comprises one or more sgRNAs targeting exon 13 of cIL6R.

In some embodiments, the CRISPR gene-editing system for the treatment of OA comprises one or more sgRNAs targeting exon 1 of eIL6R. In some embodiments, the CRISPR gene-editing system for the treatment of OA comprises one or more sgRNAs targeting exon 2 of eIL6R. In some embodiments, the CRISPR gene-editing system for the treatment of OA comprises one or more sgRNAs targeting exon 3 of eIL6R. In some embodiments, the CRISPR gene-editing system for the treatment of OA comprises one or more sgRNAs targeting exon 4 of eIL6R. In some embodiments, the CRISPR gene-editing system for the treatment of OA comprises one or more sgRNAs targeting exon 5 of eIL6R. In some embodiments, the CRISPR gene-editing system for the treatment of OA comprises one or more sgRNAs targeting exon 6 of eIL6R. In some embodiments, the CRISPR gene-editing system for the treatment of OA comprises one or more sgRNAs targeting exon 7 of eIL6R. In some embodiments, the CRISPR gene-editing system for the treatment of OA comprises one or more sgRNAs targeting exon 8 of eIL6R. In some embodiments, the CRISPR gene-editing system for the treatment of OA comprises one or more sgRNAs targeting exon 9 of eIL6R. In some embodiments, the CRISPR gene-editing system for the treatment of OA comprises one or more sgRNAs targeting exon 10 of eIL6R. In some embodiments, the CRISPR gene-editing system for the treatment of OA comprises one or more sgRNAs targeting exon 11 of eIL6R.

In some embodiments, the CRISPR gene-editing system for the treatment of OA comprises one or more sgRNAs targeting exon 1 of fIL6R. In some embodiments, the CRISPR gene-editing system for the treatment of OA comprises one or more sgRNAs targeting exon 2 of fIL6R. In some embodiments, the CRISPR gene-editing system for the treatment of OA comprises one or more sgRNAs targeting exon 3 of fIL6R. In some embodiments, the CRISPR gene-editing system for the treatment of OA comprises one or more sgRNAs targeting exon 4 of fIL6R. In some embodiments, the CRISPR gene-editing system for the treatment of OA comprises one or more sgRNAs targeting exon 5 of fIL6R. In some embodiments, the CRISPR gene-editing system for the treatment of OA comprises one or more sgRNAs targeting exon 6 of fIL6R. In some embodiments, the CRISPR gene-editing system for the treatment of OA comprises one or more sgRNAs targeting exon 7 of fIL6R. In some embodiments, the CRISPR gene-editing system for the treatment of OA comprises one or more sgRNAs targeting exon 8 of fIL6R. In some embodiments, the CRISPR gene-editing system for the treatment of OA comprises one or more sgRNAs targeting exon 9 of fIL6R. In some embodiments, the CRISPR gene-editing system for the treatment of OA comprises one or more sgRNAs targeting exon 10 of fIL6R.

In some embodiments, the CRISPR gene-editing system for the treatment of OA comprises one or more sgRNAs targeting IL6ST. In some embodiments, the CRISPR gene-editing system for the treatment of OA comprises one or more sgRNAs targeting hIL6ST. In some embodiments, the CRISPR gene-editing system for the treatment of OA comprises one or more sgRNAs targeting cIL6ST. In some embodiments, the CRISPR gene-editing system for the treatment of OA comprises one or more sgRNAs targeting eIL6ST. In some embodiments, the CRISPR gene-editing system for the treatment of OA comprises one or more sgRNAs targeting fIL6ST.

In some embodiments, the CRISPR gene-editing system for the treatment of OA comprises one or more sgRNAs targeting exon 1 of hIL6ST. In some embodiments, the CRISPR gene-editing system for the treatment of OA comprises one or more sgRNAs targeting exon 2 of hIL6ST. In some embodiments, the CRISPR gene-editing system for the treatment of OA comprises one or more sgRNAs targeting exon 3 of hIL6ST. In some embodiments, the CRISPR gene-editing system for the treatment of OA comprises one or more sgRNAs targeting exon 4 of hIL6ST. In some embodiments, the CRISPR gene-editing system for the treatment of OA comprises one or more sgRNAs targeting exon 5 of hIL6ST. In some embodiments, the CRISPR gene-editing system for the treatment of OA comprises one or more sgRNAs targeting exon 6 of hIL6ST. In some embodiments, the CRISPR gene-editing system for the treatment of OA comprises one or more sgRNAs targeting exon 7 of hIL6ST. In some embodiments, the CRISPR gene-editing system for the treatment of OA comprises one or more sgRNAs targeting exon 8 of hIL6ST. In some embodiments, the CRISPR gene-editing system for the treatment of OA comprises one or more sgRNAs targeting exon 9 of hIL6ST. In some embodiments, the CRISPR gene-editing system for the treatment of OA comprises one or more sgRNAs targeting exon 10 of hIL6ST. In some embodiments, the CRISPR gene-editing system for the treatment of OA comprises one or more sgRNAs targeting exon 11 of hIL6ST. In some embodiments, the CRISPR gene-editing system for the treatment of OA comprises one or more sgRNAs targeting exon 12 of hIL6ST. In some embodiments, the CRISPR gene-editing system for the treatment of OA comprises one or more sgRNAs targeting exon 13 of hIL6ST. In some embodiments, the CRISPR gene-editing system for the treatment of OA comprises one or more sgRNAs targeting exon 14 of hIL6ST. In some embodiments, the CRISPR gene-editing system for the treatment of OA comprises one or more sgRNAs targeting exon 15 of hIL6ST. In some embodiments, the CRISPR gene-editing system for the treatment of OA comprises one or more sgRNAs targeting exon 16 of hIL6ST. In some embodiments, the CRISPR gene-editing system for the treatment of OA comprises one or more sgRNAs targeting exon 17 of hIL6ST. In some embodiments, the CRISPR gene-editing system for the treatment of OA comprises one or more sgRNAs targeting exon 18 of hIL6ST. In some embodiments, the CRISPR gene-editing system for the treatment of OA comprises one or more sgRNAs targeting exon 19 of hIL6ST.

In some embodiments, the CRISPR gene-editing system for the treatment of OA comprises one or more sgRNAs targeting exon 1 of cIL6ST. In some embodiments, the CRISPR gene-editing system for the treatment of OA comprises one or more sgRNAs targeting exon 2 of cIL6ST. In some embodiments, the CRISPR gene-editing system for the treatment of OA comprises one or more sgRNAs targeting exon 3 of cIL6ST. In some embodiments, the CRISPR gene-editing system for the treatment of OA comprises one or more sgRNAs targeting exon 4 of cIL6ST. In some embodiments, the CRISPR gene-editing system for the treatment of OA comprises one or more sgRNAs targeting exon 5 of cIL6ST. In some embodiments, the CRISPR gene-editing system for the treatment of OA comprises one or more sgRNAs targeting exon 6 of cIL6ST. In some embodiments, the CRISPR gene-editing system for the treatment of OA comprises one or more sgRNAs targeting exon 7 of cIL6ST. In some embodiments, the CRISPR gene-editing system for the treatment of OA comprises one or more sgRNAs targeting exon 8 of cIL6ST. In some embodiments, the CRISPR gene-editing system for the treatment of OA comprises one or more sgRNAs targeting exon 9 of cIL6ST. In some embodiments, the CRISPR gene-editing system for the treatment of OA comprises one or more sgRNAs targeting exon 10 of cIL6ST. In some embodiments, the CRISPR gene-editing system for the treatment of OA comprises one or more sgRNAs targeting exon 11 of cIL6ST In some embodiments, the CRISPR gene-editing system for the treatment of OA comprises one or more sgRNAs targeting exon 12 of cIL6ST. In some embodiments, the CRISPR gene-editing system for the treatment of OA comprises one or more sgRNAs targeting exon 13 of cIL6ST. In some embodiments, the CRISPR gene-editing system for the treatment of OA comprises one or more sgRNAs targeting exon 14 of cIL6ST. In some embodiments, the CRISPR gene-editing system for the treatment of OA comprises one or more sgRNAs targeting exon 15 of cIL6ST. In some embodiments, the CRISPR gene-editing system for the treatment of OA comprises one or more sgRNAs targeting exon 16 of cIL6ST. In some embodiments, the CRISPR gene-editing system for the treatment of OA comprises one or more sgRNAs targeting exon 17 of cIL6ST. In some embodiments, the CRISPR gene-editing system for the treatment of OA comprises one or more sgRNAs targeting exon 18 of cIL6ST.

In some embodiments, the CRISPR gene-editing system for the treatment of OA comprises one or more sgRNAs targeting exon 1 of eIL6ST. In some embodiments, the CRISPR gene-editing system for the treatment of OA comprises one or more sgRNAs targeting exon 2 of eIL6ST. In some embodiments, the CRISPR gene-editing system for the treatment of OA comprises one or more sgRNAs targeting exon 3 of eIL6ST. In some embodiments, the CRISPR gene-editing system for the treatment of OA comprises one or more sgRNAs targeting exon 4 of eIL6ST. In some embodiments, the CRISPR gene-editing system for the treatment of OA comprises one or more sgRNAs targeting exon 5 of eIL6ST. In some embodiments, the CRISPR gene-editing system for the treatment of OA comprises one or more sgRNAs targeting exon 6 of eIL6ST. In some embodiments, the CRISPR gene-editing system for the treatment of OA comprises one or more sgRNAs targeting exon 7 of eIL6ST. In some embodiments, the CRISPR gene-editing system for the treatment of OA comprises one or more sgRNAs targeting exon 8 of eIL6ST. In some embodiments, the CRISPR gene-editing system for the treatment of OA comprises one or more sgRNAs targeting exon 9 of eIL6ST. In some embodiments, the CRISPR gene-editing system for the treatment of OA comprises one or more sgRNAs targeting exon 10 of eIL6ST. In some embodiments, the CRISPR gene-editing system for the treatment of OA comprises one or more sgRNAs targeting exon 11 of eIL6ST In some embodiments, the CRISPR gene-editing system for the treatment of OA comprises one or more sgRNAs targeting exon 12 of eIL6ST. In some embodiments, the CRISPR gene-editing system for the treatment of OA comprises one or more sgRNAs targeting exon 13 of eIL6ST. In some embodiments, the CRISPR gene-editing system for the treatment of OA comprises one or more sgRNAs targeting exon 14 of eIL6ST. In some embodiments, the CRISPR gene-editing system for the treatment of OA comprises one or more sgRNAs targeting exon 15 of eIL6ST. In some embodiments, the CRISPR gene-editing system for the treatment of OA comprises one or more sgRNAs targeting exon 16 of eIL6ST. In some embodiments, the CRISPR gene-editing system for the treatment of OA comprises one or more sgRNAs targeting exon 17 of eIL6ST. In some embodiments, the CRISPR gene-editing system for the treatment of OA comprises one or more sgRNAs targeting exon 18 of eIL6ST.

In some embodiments, the CRISPR gene-editing system for the treatment of OA comprises one or more sgRNAs targeting exon 1 of fIL6ST. In some embodiments, the CRISPR gene-editing system for the treatment of OA comprises one or more sgRNAs targeting exon 2 of fIL6ST. In some embodiments, the CRISPR gene-editing system for the treatment of OA comprises one or more sgRNAs targeting exon 3 of fIL6ST. In some embodiments, the CRISPR gene-editing system for the treatment of OA comprises one or more sgRNAs targeting exon 4 of fIL6ST. In some embodiments, the CRISPR gene-editing system for the treatment of OA comprises one or more sgRNAs targeting exon 5 of fIL6ST. In some embodiments, the CRISPR gene-editing system for the treatment of OA comprises one or more sgRNAs targeting exon 6 of fIL6ST. In some embodiments, the CRISPR gene-editing system for the treatment of OA comprises one or more sgRNAs targeting exon 7 of fIL6ST. In some embodiments, the CRISPR gene-editing system for the treatment of OA comprises one or more sgRNAs targeting exon 8 of fIL6ST. In some embodiments, the CRISPR gene-editing system for the treatment of OA comprises one or more sgRNAs targeting exon 9 of fIL6ST. In some embodiments, the CRISPR gene-editing system for the treatment of OA comprises one or more sgRNAs targeting exon 10 of fIL6ST. In some embodiments, the CRISPR gene-editing system for the treatment of OA comprises one or more sgRNAs targeting exon 11 of fIL6ST In some embodiments, the CRISPR gene-editing system for the treatment of OA comprises one or more sgRNAs targeting exon 12 of fIL6ST. In some embodiments, the CRISPR gene-editing system for the treatment of OA comprises one or more sgRNAs targeting exon 13 of fIL6ST. In some embodiments, the CRISPR gene-editing system for the treatment of OA comprises one or more sgRNAs targeting exon 14 of fIL6ST. In some embodiments, the CRISPR gene-editing system for the treatment of OA comprises one or more sgRNAs targeting exon 15 of fIL6ST. In some embodiments, the CRISPR gene-editing system for the treatment of OA comprises one or more sgRNAs targeting exon 16 of fIL6ST. In some embodiments, the CRISPR gene-editing system for the treatment of OA comprises one or more sgRNAs targeting exon 17 of fIL6ST. In some embodiments, the CRISPR gene-editing system for the treatment of OA comprises one or more sgRNAs targeting exon 18 of fIL6ST. In some embodiments, the CRISPR gene-editing system for the treatment of OA comprises one or more sgRNAs targeting exon 19 of fIL6ST. In some embodiments, the CRISPR gene-editing system for the treatment of OA comprises one or more sgRNAs targeting exon 20 of fIL6ST.

In some embodiments, the CRISPR gene-editing system for the treatment of OA comprises one or more sgRNAs targeting TNFRSF1A. In some embodiments, the CRISPR gene-editing system for the treatment of OA comprises one or more sgRNAs targeting hTNFRSF1A. In some embodiments, the CRISPR gene-editing system for the treatment of OA comprises one or more sgRNAs targeting cTNFRSF1A. In some embodiments, the CRISPR gene-editing system for the treatment of OA comprises one or more sgRNAs targeting eTNFRSF1A. In some embodiments, the CRISPR gene-editing system for the treatment of OA comprises one or more sgRNAs targeting fTNFRSF1A.

In some embodiments, the CRISPR gene-editing system for the treatment of OA comprises one or more sgRNAs targeting exon 1 of hTNFRSF1A. In some embodiments, the CRISPR gene-editing system for the treatment of OA comprises one or more sgRNAs targeting exon 2 of hTNFRSF1A. In some embodiments, the CRISPR gene-editing system for the treatment of OA comprises one or more sgRNAs targeting exon 3 of hTNFRSF1A. In some embodiments, the CRISPR gene-editing system for the treatment of OA comprises one or more sgRNAs targeting exon 4 of hTNFRSF1A. In some embodiments, the CRISPR gene-editing system for the treatment of OA comprises one or more sgRNAs targeting exon 5 of hTNFRSF1A. In some embodiments, the CRISPR gene-editing system for the treatment of OA comprises one or more sgRNAs targeting exon 6 of hTNFRSF1A. In some embodiments, the CRISPR gene-editing system for the treatment of OA comprises one or more sgRNAs targeting exon 7 of hTNFRSF1A. In some embodiments, the CRISPR gene-editing system for the treatment of OA comprises one or more sgRNAs targeting exon 8 of hTNFRSF1A. In some embodiments, the CRISPR gene-editing system for the treatment of OA comprises one or more sgRNAs targeting exon 9 of hTNFRSF1A. In some embodiments, the CRISPR gene-editing system for the treatment of OA comprises one or more sgRNAs targeting exon 10 of hTNFRSF1A. In some embodiments, the CRISPR gene-editing system for the treatment of OA comprises one or more sgRNAs targeting exon 11 of hTNFRSF1A.

In some embodiments, the CRISPR gene-editing system for the treatment of OA comprises one or more sgRNAs targeting exon 1 of cTNFRSF1A. In some embodiments, the CRISPR gene-editing system for the treatment of OA comprises one or more sgRNAs targeting exon 2 of cTNFRSF1A. In some embodiments, the CRISPR gene-editing system for the treatment of OA comprises one or more sgRNAs targeting exon 3 of cTNFRSF1A. In some embodiments, the CRISPR gene-editing system for the treatment of OA comprises one or more sgRNAs targeting exon 4 of cTNFRSF1A. In some embodiments, the CRISPR gene-editing system for the treatment of OA comprises one or more sgRNAs targeting exon 5 of cTNFRSF1A. In some embodiments, the CRISPR gene-editing system for the treatment of OA comprises one or more sgRNAs targeting exon 6 of cTNFRSF1A. In some embodiments, the CRISPR gene-editing system for the treatment of OA comprises one or more sgRNAs targeting exon 7 of cTNFRSF1A. In some embodiments, the CRISPR gene-editing system for the treatment of OA comprises one or more sgRNAs targeting exon 8 of cTNFRSF1A. In some embodiments, the CRISPR gene-editing system for the treatment of OA comprises one or more sgRNAs targeting exon 9 of cTNFRSF1A. In some embodiments, the CRISPR gene-editing system for the treatment of OA comprises one or more sgRNAs targeting exon 10 of cTNFRSF1A. In some embodiments, the CRISPR gene-editing system for the treatment of OA comprises one or more sgRNAs targeting exon 11 of cTNFRSF1A.

In some embodiments, the CRISPR gene-editing system for the treatment of OA comprises one or more sgRNAs targeting exon 1 of eTNFRSF1A. In some embodiments, the CRISPR gene-editing system for the treatment of OA comprises one or more sgRNAs targeting exon 2 of eTNFRSF1A. In some embodiments, the CRISPR gene-editing system for the treatment of OA comprises one or more sgRNAs targeting exon 3 of eTNFRSF1A. In some embodiments, the CRISPR gene-editing system for the treatment of OA comprises one or more sgRNAs targeting exon 4 of eTNFRSF1A. In some embodiments, the CRISPR gene-editing system for the treatment of OA comprises one or more sgRNAs targeting exon 5 of eTNFRSF1A. In some embodiments, the CRISPR gene-editing system for the treatment of OA comprises one or more sgRNAs targeting exon 6 of eTNFRSF1A. In some embodiments, the CRISPR gene-editing system for the treatment of OA comprises one or more sgRNAs targeting exon 7 of eTNFRSF1A. In some embodiments, the CRISPR gene-editing system for the treatment of OA comprises one or more sgRNAs targeting exon 8 of eTNFRSF1A. In some embodiments, the CRISPR gene-editing system for the treatment of OA comprises one or more sgRNAs targeting exon 9 of eTNFRSF1A. In some embodiments, the CRISPR gene-editing system for the treatment of OA comprises one or more sgRNAs targeting exon 10 of eTNFRSF1A.

In some embodiments, the CRISPR gene-editing system for the treatment of OA comprises one or more sgRNAs targeting exon 1 of fTNFRSF1A. In some embodiments, the CRISPR gene-editing system for the treatment of OA comprises one or more sgRNAs targeting exon 2 of fTNFRSF1A. In some embodiments, the CRISPR gene-editing system for the treatment of OA comprises one or more sgRNAs targeting exon 3 of fTNFRSF1A. In some embodiments, the CRISPR gene-editing system for the treatment of OA comprises one or more sgRNAs targeting exon 4 of fTNFRSF1A. In some embodiments, the CRISPR gene-editing system for the treatment of OA comprises one or more sgRNAs targeting exon 5 of fTNFRSF1A. In some embodiments, the CRISPR gene-editing system for the treatment of OA comprises one or more sgRNAs targeting exon 6 of fTNFRSF1A. In some embodiments, the CRISPR gene-editing system for the treatment of OA comprises one or more sgRNAs targeting exon 7 of fTNFRSF1A. In some embodiments, the CRISPR gene-editing system for the treatment of OA comprises one or more sgRNAs targeting exon 8 of fTNFRSF1A. In some embodiments, the CRISPR gene-editing system for the treatment of OA comprises one or more sgRNAs targeting exon 9 of fTNFRSF1A. In some embodiments, the CRISPR gene-editing system for the treatment of OA comprises one or more sgRNAs targeting exon 10 of fTNFRSF1A.

In some embodiments, the CRISPR gene-editing system for the treatment of OA comprises one or more sgRNAs targeting TNFRSF1B. In some embodiments, the CRISPR gene-editing system for the treatment of OA comprises one or more sgRNAs targeting hTNFRSF1B. In some embodiments, the CRISPR gene-editing system for the treatment of OA comprises one or more sgRNAs targeting cTNFRSF1B. In some embodiments, the CRISPR gene-editing system for the treatment of OA comprises one or more sgRNAs targeting eTNFRSF1B. In some embodiments, the CRISPR gene-editing system for the treatment of OA comprises one or more sgRNAs targeting fTNFRSF1B.

In some embodiments, the CRISPR gene-editing system for the treatment of OA comprises one or more sgRNAs targeting exon 1 of hTNFRSF1B. In some embodiments, the CRISPR gene-editing system for the treatment of OA comprises one or more sgRNAs targeting exon 2 of hTNFRSF1B. In some embodiments, the CRISPR gene-editing system for the treatment of OA comprises one or more sgRNAs targeting exon 3 of hTNFRSF1B. In some embodiments, the CRISPR gene-editing system for the treatment of OA comprises one or more sgRNAs targeting exon 4 of hTNFRSF1B. In some embodiments, the CRISPR gene-editing system for the treatment of OA comprises one or more sgRNAs targeting exon 5 of hTNFRSF1B. In some embodiments, the CRISPR gene-editing system for the treatment of OA comprises one or more sgRNAs targeting exon 6 of hTNFRSF1B. In some embodiments, the CRISPR gene-editing system for the treatment of OA comprises one or more sgRNAs targeting exon 7 of hTNFRSF1B. In some embodiments, the CRISPR gene-editing system for the treatment of OA comprises one or more sgRNAs targeting exon 8 of hTNFRSF1B. In some embodiments, the CRISPR gene-editing system for the treatment of OA comprises one or more sgRNAs targeting exon 9 of hTNFRSF1B. In some embodiments, the CRISPR gene-editing system for the treatment of OA comprises one or more sgRNAs targeting exon 10 of hTNFRSF1B. In some embodiments, the CRISPR gene-editing system for the treatment of OA comprises one or more sgRNAs targeting exon 11 of hTNFRSF1B In some embodiments, the CRISPR gene-editing system for the treatment of OA comprises one or more sgRNAs targeting exon 12 of hTNFRSF1B. In some embodiments, the CRISPR gene-editing system for the treatment of OA comprises one or more sgRNAs targeting exon 13 of hTNFRSF1B.

In some embodiments, the CRISPR gene-editing system for the treatment of OA comprises one or more sgRNAs targeting exon 1 of cTNFRSF1B. In some embodiments, the CRISPR gene-editing system for the treatment of OA comprises one or more sgRNAs targeting exon 2 of cTNFRSF1B. In some embodiments, the CRISPR gene-editing system for the treatment of OA comprises one or more sgRNAs targeting exon 3 of cTNFRSF1B. In some embodiments, the CRISPR gene-editing system for the treatment of OA comprises one or more sgRNAs targeting exon 4 of cTNFRSF1B. In some embodiments, the CRISPR gene-editing system for the treatment of OA comprises one or more sgRNAs targeting exon 5 of cTNFRSF1B. In some embodiments, the CRISPR gene-editing system for the treatment of OA comprises one or more sgRNAs targeting exon 6 of cTNFRSF1B. In some embodiments, the CRISPR gene-editing system for the treatment of OA comprises one or more sgRNAs targeting exon 7 of cTNFRSF1B. In some embodiments, the CRISPR gene-editing system for the treatment of OA comprises one or more sgRNAs targeting exon 8 of cTNFRSF1B. In some embodiments, the CRISPR gene-editing system for the treatment of OA comprises one or more sgRNAs targeting exon 9 of cTNFRSF1B. In some embodiments, the CRISPR gene-editing system for the treatment of OA comprises one or more sgRNAs targeting exon 10 of cTNFRSF1B. In some embodiments, the CRISPR gene-editing system for the treatment of OA comprises one or more sgRNAs targeting exon 11 of cTNFRSF1B.

In some embodiments, the CRISPR gene-editing system for the treatment of OA comprises one or more sgRNAs targeting exon 1 of eTNFRSF1B. In some embodiments, the CRISPR gene-editing system for the treatment of OA comprises one or more sgRNAs targeting exon 2 of eTNFRSF1B. In some embodiments, the CRISPR gene-editing system for the treatment of OA comprises one or more sgRNAs targeting exon 3 of eTNFRSF1B. In some embodiments, the CRISPR gene-editing system for the treatment of OA comprises one or more sgRNAs targeting exon 4 of eTNFRSF1B. In some embodiments, the CRISPR gene-editing system for the treatment of OA comprises one or more sgRNAs targeting exon 5 of eTNFRSF1B. In some embodiments, the CRISPR gene-editing system for the treatment of OA comprises one or more sgRNAs targeting exon 6 of eTNFRSF1B. In some embodiments, the CRISPR gene-editing system for the treatment of OA comprises one or more sgRNAs targeting exon 7 of eTNFRSF1B. In some embodiments, the CRISPR gene-editing system for the treatment of OA comprises one or more sgRNAs targeting exon 8 of eTNFRSF1B. In some embodiments, the CRISPR gene-editing system for the treatment of OA comprises one or more sgRNAs targeting exon 9 of eTNFRSF1B. In some embodiments, the CRISPR gene-editing system for the treatment of OA comprises one or more sgRNAs targeting exon 10 of eTNFRSF1B. In some embodiments, the CRISPR gene-editing system for the treatment of OA comprises one or more sgRNAs targeting exon 11 of eTNFRSF1B.

In some embodiments, the CRISPR gene-editing system for the treatment of OA comprises one or more sgRNAs targeting exon 1 of fTNFRSF1B. In some embodiments, the CRISPR gene-editing system for the treatment of OA comprises one or more sgRNAs targeting exon 2 of fTNFRSF1B. In some embodiments, the CRISPR gene-editing system for the treatment of OA comprises one or more sgRNAs targeting exon 3 of fTNFRSF1B. In some embodiments, the CRISPR gene-editing system for the treatment of OA comprises one or more sgRNAs targeting exon 4 of fTNFRSF1B. In some embodiments, the CRISPR gene-editing system for the treatment of OA comprises one or more sgRNAs targeting exon 5 of fTNFRSF1B. In some embodiments, the CRISPR gene-editing system for the treatment of OA comprises one or more sgRNAs targeting exon 6 of fTNFRSF1B. In some embodiments, the CRISPR gene-editing system for the treatment of OA comprises one or more sgRNAs targeting exon 7 of fTNFRSF1B. In some embodiments, the CRISPR gene-editing system for the treatment of OA comprises one or more sgRNAs targeting exon 8 of fTNFRSF1B. In some embodiments, the CRISPR gene-editing system for the treatment of OA comprises one or more sgRNAs targeting exon 9 of fTNFRSF1B. In some embodiments, the CRISPR gene-editing system for the treatment of OA comprises one or more sgRNAs targeting exon 10 of fTNFRSF1B.

In some embodiments, the CRISPR gene-editing system for the treatment of OA comprises one or more sgRNAs targeting TNFRSF3. In some embodiments, the CRISPR gene-editing system for the treatment of OA comprises one or more sgRNAs targeting hTNFRSF3. In some embodiments, the CRISPR gene-editing system for the treatment of OA comprises one or more sgRNAs targeting cTNFRSF3. In some embodiments, the CRISPR gene-editing system for the treatment of OA comprises one or more sgRNAs targeting eTNFRSF3. In some embodiments, the CRISPR gene-editing system for the treatment of OA comprises one or more sgRNAs targeting fTNFRSF3.

In some embodiments, the CRISPR gene-editing system for the treatment of OA comprises one or more sgRNAs targeting exon 1 of hTNFRSF3. In some embodiments, the CRISPR gene-editing system for the treatment of OA comprises one or more sgRNAs targeting exon 2 of hTNFRSF3. In some embodiments, the CRISPR gene-editing system for the treatment of OA comprises one or more sgRNAs targeting exon 3 of hTNFRSF3. In some embodiments, the CRISPR gene-editing system for the treatment of OA comprises one or more sgRNAs targeting exon 4 of hTNFRSF3. In some embodiments, the CRISPR gene-editing system for the treatment of OA comprises one or more sgRNAs targeting exon 5 of hTNFRSF3. In some embodiments, the CRISPR gene-editing system for the treatment of OA comprises one or more sgRNAs targeting exon 6 of hTNFRSF3. In some embodiments, the CRISPR gene-editing system for the treatment of OA comprises one or more sgRNAs targeting exon 7 of hTNFRSF3. In some embodiments, the CRISPR gene-editing system for the treatment of OA comprises one or more sgRNAs targeting exon 8 of hTNFRSF3. In some embodiments, the CRISPR gene-editing system for the treatment of OA comprises one or more sgRNAs targeting exon 9 of hTNFRSF3. In some embodiments, the CRISPR gene-editing system for the treatment of OA comprises one or more sgRNAs targeting exon 10 of hTNFRSF3. In some embodiments, the CRISPR gene-editing system for the treatment of OA comprises one or more sgRNAs targeting exon 11 of hTNFRSF3. In some embodiments, the CRISPR gene-editing system for the treatment of OA comprises one or more sgRNAs targeting exon 12 of hTNFRSF3.

In some embodiments, the CRISPR gene-editing system for the treatment of OA comprises one or more sgRNAs targeting exon 1 of cTNFRSF3. In some embodiments, the CRISPR gene-editing system for the treatment of OA comprises one or more sgRNAs targeting exon 2 of cTNFRSF3. In some embodiments, the CRISPR gene-editing system for the treatment of OA comprises one or more sgRNAs targeting exon 3 of cTNFRSF3. In some embodiments, the CRISPR gene-editing system for the treatment of OA comprises one or more sgRNAs targeting exon 4 of cTNFRSF3. In some embodiments, the CRISPR gene-editing system for the treatment of OA comprises one or more sgRNAs targeting exon 5 of cTNFRSF3. In some embodiments, the CRISPR gene-editing system for the treatment of OA comprises one or more sgRNAs targeting exon 6 of cTNFRSF3. In some embodiments, the CRISPR gene-editing system for the treatment of OA comprises one or more sgRNAs targeting exon 7 of cTNFRSF3. In some embodiments, the CRISPR gene-editing system for the treatment of OA comprises one or more sgRNAs targeting exon 8 of cTNFRSF3. In some embodiments, the CRISPR gene-editing system for the treatment of OA comprises one or more sgRNAs targeting exon 9 of cTNFRSF3. In some embodiments, the CRISPR gene-editing system for the treatment of OA comprises one or more sgRNAs targeting exon 10 of cTNFRSF3.

In some embodiments, the CRISPR gene-editing system for the treatment of OA comprises one or more sgRNAs targeting exon 1 of eTNFRSF3. In some embodiments, the CRISPR gene-editing system for the treatment of OA comprises one or more sgRNAs targeting exon 2 of eTNFRSF3. In some embodiments, the CRISPR gene-editing system for the treatment of OA comprises one or more sgRNAs targeting exon 3 of eTNFRSF3. In some embodiments, the CRISPR gene-editing system for the treatment of OA comprises one or more sgRNAs targeting exon 4 of eTNFRSF3. In some embodiments, the CRISPR gene-editing system for the treatment of OA comprises one or more sgRNAs targeting exon 5 of eTNFRSF3. In some embodiments, the CRISPR gene-editing system for the treatment of OA comprises one or more sgRNAs targeting exon 6 of eTNFRSF3. In some embodiments, the CRISPR gene-editing system for the treatment of OA comprises one or more sgRNAs targeting exon 7 of eTNFRSF3. In some embodiments, the CRISPR gene-editing system for the treatment of OA comprises one or more sgRNAs targeting exon 8 of eTNFRSF3. In some embodiments, the CRISPR gene-editing system for the treatment of OA comprises one or more sgRNAs targeting exon 9 of eTNFRSF3. In some embodiments, the CRISPR gene-editing system for the treatment of OA comprises one or more sgRNAs targeting exon 10 of eTNFRSF3.

In some embodiments, the CRISPR gene-editing system for the treatment of OA comprises one or more sgRNAs targeting exon 1 of fTNFRSF3. In some embodiments, the CRISPR gene-editing system for the treatment of OA comprises one or more sgRNAs targeting exon 2 of fTNFRSF3. In some embodiments, the CRISPR gene-editing system for the treatment of OA comprises one or more sgRNAs targeting exon 3 of fTNFRSF3. In some embodiments, the CRISPR gene-editing system for the treatment of OA comprises one or more sgRNAs targeting exon 4 of fTNFRSF3. In some embodiments, the CRISPR gene-editing system for the treatment of OA comprises one or more sgRNAs targeting exon 5 of fTNFRSF3. In some embodiments, the CRISPR gene-editing system for the treatment of OA comprises one or more sgRNAs targeting exon 6 of fTNFRSF3. In some embodiments, the CRISPR gene-editing system for the treatment of OA comprises one or more sgRNAs targeting exon 7 of fTNFRSF3. In some embodiments, the CRISPR gene-editing system for the treatment of OA comprises one or more sgRNAs targeting exon 8 of fTNFRSF3. In some embodiments, the CRISPR gene-editing system for the treatment of OA comprises one or more sgRNAs targeting exon 9 of fTNFRSF3. In some embodiments, the CRISPR gene-editing system for the treatment of OA comprises one or more sgRNAs targeting exon 10 of fTNFRSF3.

In some embodiments, the CRISPR gene-editing system for the treatment of OA comprises one or more sgRNAs targeting TNFRSF4. In some embodiments, the CRISPR gene-editing system for the treatment of OA comprises one or more sgRNAs targeting hTNFRSF4. In some embodiments, the CRISPR gene-editing system for the treatment of OA comprises one or more sgRNAs targeting cTNFRSF4. In some embodiments, the CRISPR gene-editing system for the treatment of OA comprises one or more sgRNAs targeting eTNFRSF4. In some embodiments, the CRISPR gene-editing system for the treatment of OA comprises one or more sgRNAs targeting fTNFRSF4.

In some embodiments, the CRISPR gene-editing system for the treatment of OA comprises one or more sgRNAs targeting exon 1 of hTNFRSF4. In some embodiments, the CRISPR gene-editing system for the treatment of OA comprises one or more sgRNAs targeting exon 2 of hTNFRSF4. In some embodiments, the CRISPR gene-editing system for the treatment of OA comprises one or more sgRNAs targeting exon 3 of hTNFRSF4. In some embodiments, the CRISPR gene-editing system for the treatment of OA comprises one or more sgRNAs targeting exon 4 of hTNFRSF4. In some embodiments, the CRISPR gene-editing system for the treatment of OA comprises one or more sgRNAs targeting exon 5 of hTNFRSF4. In some embodiments, the CRISPR gene-editing system for the treatment of OA comprises one or more sgRNAs targeting exon 6 of hTNFRSF4.

In some embodiments, the CRISPR gene-editing system for the treatment of OA comprises one or more sgRNAs targeting exon 1 of cTNFRSF4. In some embodiments, the CRISPR gene-editing system for the treatment of OA comprises one or more sgRNAs targeting exon 2 of cTNFRSF4. In some embodiments, the CRISPR gene-editing system for the treatment of OA comprises one or more sgRNAs targeting exon 3 of cTNFRSF4. In some embodiments, the CRISPR gene-editing system for the treatment of OA comprises one or more sgRNAs targeting exon 4 of cTNFRSF4. In some embodiments, the CRISPR gene-editing system for the treatment of OA comprises one or more sgRNAs targeting exon 5 of cTNFRSF4. In some embodiments, the CRISPR gene-editing system for the treatment of OA comprises one or more sgRNAs targeting exon 6 of cTNFRSF4. In some embodiments, the CRISPR gene-editing system for the treatment of OA comprises one or more sgRNAs targeting exon 7 of cTNFRSF4.

In some embodiments, the CRISPR gene-editing system for the treatment of OA comprises one or more sgRNAs targeting exon 1 of eTNFRSF4. In some embodiments, the CRISPR gene-editing system for the treatment of OA comprises one or more sgRNAs targeting exon 2 of eTNFRSF4. In some embodiments, the CRISPR gene-editing system for the treatment of OA comprises one or more sgRNAs targeting exon 3 of eTNFRSF4. In some embodiments, the CRISPR gene-editing system for the treatment of OA comprises one or more sgRNAs targeting exon 4 of eTNFRSF4. In some embodiments, the CRISPR gene-editing system for the treatment of OA comprises one or more sgRNAs targeting exon 5 of eTNFRSF4. In some embodiments, the CRISPR gene-editing system for the treatment of OA comprises one or more sgRNAs targeting exon 6 of eTNFRSF4. In some embodiments, the CRISPR gene-editing system for the treatment of OA comprises one or more sgRNAs targeting exon 7 of eTNFRSF4.

In some embodiments, the CRISPR gene-editing system for the treatment of OA comprises one or more sgRNAs targeting exon 1 of fTNFRSF4. In some embodiments, the CRISPR gene-editing system for the treatment of OA comprises one or more sgRNAs targeting exon 2 of fTNFRSF4. In some embodiments, the CRISPR gene-editing system for the treatment of OA comprises one or more sgRNAs targeting exon 3 of fTNFRSF4. In some embodiments, the CRISPR gene-editing system for the treatment of OA comprises one or more sgRNAs targeting exon 4 of fTNFRSF4. In some embodiments, the CRISPR gene-editing system for the treatment of OA comprises one or more sgRNAs targeting exon 5 of fTNFRSF4. In some embodiments, the CRISPR gene-editing system for the treatment of OA comprises one or more sgRNAs targeting exon 6 of fTNFRSF4. In some embodiments, the CRISPR gene-editing system for the treatment of OA comprises one or more sgRNAs targeting exon 7 of fTNFRSF4.

In some embodiments, the CRISPR gene-editing system for the treatment of OA comprises one or more sgRNAs targeting TNFRSF11A. In some embodiments, the CRISPR gene-editing system for the treatment of OA comprises one or more sgRNAs targeting hTNFRSF11A. In some embodiments, the CRISPR gene-editing system for the treatment of OA comprises one or more sgRNAs targeting cTNFRSF11A. In some embodiments, the CRISPR gene-editing system for the treatment of OA comprises one or more sgRNAs targeting eTNFRSF11A. In some embodiments, the CRISPR gene-editing system for the treatment of OA comprises one or more sgRNAs targeting fTNFRSF11A.

In some embodiments, the CRISPR gene-editing system for the treatment of OA comprises one or more sgRNAs targeting exon 1 of hTNFRSF11A. In some embodiments, the CRISPR gene-editing system for the treatment of OA comprises one or more sgRNAs targeting exon 2 of hTNFRSF11A. In some embodiments, the CRISPR gene-editing system for the treatment of OA comprises one or more sgRNAs targeting exon 3 of hTNFRSF11A. In some embodiments, the CRISPR gene-editing system for the treatment of OA comprises one or more sgRNAs targeting exon 4 of hTNFRSF11A. In some embodiments, the CRISPR gene-editing system for the treatment of OA comprises one or more sgRNAs targeting exon 5 of hTNFRSF11A. In some embodiments, the CRISPR gene-editing system for the treatment of OA comprises one or more sgRNAs targeting exon 6 of hTNFRSF11A. In some embodiments, the CRISPR gene-editing system for the treatment of OA comprises one or more sgRNAs targeting exon 7 of hTNFRSF11A. In some embodiments, the CRISPR gene-editing system for the treatment of OA comprises one or more sgRNAs targeting exon 8 of hTNFRSF11A. In some embodiments, the CRISPR gene-editing system for the treatment of OA comprises one or more sgRNAs targeting exon 9 of hTNFRSF11A. In some embodiments, the CRISPR gene-editing system for the treatment of OA comprises one or more sgRNAs targeting exon 10 of hTNFRSF11A. In some embodiments, the CRISPR gene-editing system for the treatment of OA comprises one or more sgRNAs targeting exon 11 of hTNFRSF11A. In some embodiments, the CRISPR gene-editing system for the treatment of OA comprises one or more sgRNAs targeting exon 12 of hTNFRSF11A.

In some embodiments, the CRISPR gene-editing system for the treatment of OA comprises one or more sgRNAs targeting exon 1 of cTNFRSF11A. In some embodiments, the CRISPR gene-editing system for the treatment of OA comprises one or more sgRNAs targeting exon 2 of cTNFRSF11A. In some embodiments, the CRISPR gene-editing system for the treatment of OA comprises one or more sgRNAs targeting exon 3 of cTNFRSF11A. In some embodiments, the CRISPR gene-editing system for the treatment of OA comprises one or more sgRNAs targeting exon 4 of cTNFRSF11A. In some embodiments, the CRISPR gene-editing system for the treatment of OA comprises one or more sgRNAs targeting exon 5 of cTNFRSF11A. In some embodiments, the CRISPR gene-editing system for the treatment of OA comprises one or more sgRNAs targeting exon 6 of cTNFRSF11A. In some embodiments, the CRISPR gene-editing system for the treatment of OA comprises one or more sgRNAs targeting exon 7 of cTNFRSF11A. In some embodiments, the CRISPR gene-editing system for the treatment of OA comprises one or more sgRNAs targeting exon 8 of cTNFRSF11A. In some embodiments, the CRISPR gene-editing system for the treatment of OA comprises one or more sgRNAs targeting exon 9 of cTNFRSF11A. In some embodiments, the CRISPR gene-editing system for the treatment of OA comprises one or more sgRNAs targeting exon 10 of cTNFRSF11A. In some embodiments, the CRISPR gene-editing system for the treatment of OA comprises one or more sgRNAs targeting exon 11 of cTNFRSF11A.

In some embodiments, the CRISPR gene-editing system for the treatment of OA comprises one or more sgRNAs targeting exon 1 of eTNFRSF11A. In some embodiments, the CRISPR gene-editing system for the treatment of OA comprises one or more sgRNAs targeting exon 2 of eTNFRSF11A. In some embodiments, the CRISPR gene-editing system for the treatment of OA comprises one or more sgRNAs targeting exon 3 of eTNFRSF11A. In some embodiments, the CRISPR gene-editing system for the treatment of OA comprises one or more sgRNAs targeting exon 4 of eTNFRSF11A. In some embodiments, the CRISPR gene-editing system for the treatment of OA comprises one or more sgRNAs targeting exon 5 of eTNFRSF11A. In some embodiments, the CRISPR gene-editing system for the treatment of OA comprises one or more sgRNAs targeting exon 6 of eTNFRSF11A. In some embodiments, the CRISPR gene-editing system for the treatment of OA comprises one or more sgRNAs targeting exon 7 of eTNFRSF11A. In some embodiments, the CRISPR gene-editing system for the treatment of OA comprises one or more sgRNAs targeting exon 8 of eTNFRSF11A. In some embodiments, the CRISPR gene-editing system for the treatment of OA comprises one or more sgRNAs targeting exon 9 of eTNFRSF11A. In some embodiments, the CRISPR gene-editing system for the treatment of OA comprises one or more sgRNAs targeting exon 10 of eTNFRSF11A.

In some embodiments, the CRISPR gene-editing system for the treatment of OA comprises one or more sgRNAs targeting exon 1 of fTNFRSF11A. In some embodiments, the CRISPR gene-editing system for the treatment of OA comprises one or more sgRNAs targeting exon 2 of fTNFRSF11A. In some embodiments, the CRISPR gene-editing system for the treatment of OA comprises one or more sgRNAs targeting exon 3 of fTNFRSF11A. In some embodiments, the CRISPR gene-editing system for the treatment of OA comprises one or more sgRNAs targeting exon 4 of fTNFRSF11A. In some embodiments, the CRISPR gene-editing system for the treatment of OA comprises one or more sgRNAs targeting exon 5 of fTNFRSF11A. In some embodiments, the CRISPR gene-editing system for the treatment of OA comprises one or more sgRNAs targeting exon 6 of fTNFRSF11A. In some embodiments, the CRISPR gene-editing system for the treatment of OA comprises one or more sgRNAs targeting exon 7 of fTNFRSF11A. In some embodiments, the CRISPR gene-editing system for the treatment of OA comprises one or more sgRNAs targeting exon 8 of fTNFRSF11A. In some embodiments, the CRISPR gene-editing system for the treatment of OA comprises one or more sgRNAs targeting exon 9 of fTNFRSF11A. In some embodiments, the CRISPR gene-editing system for the treatment of OA comprises one or more sgRNAs targeting exon 10 of fTNFRSF11A. In some embodiments, the CRISPR gene-editing system for the treatment of OA comprises one or more sgRNAs targeting exon 11 of fTNFRSF11A.

### C. Psoriatic Arthritis

In one aspect, the present disclosure encompasses treatments for psoriatic arthritis (PsA). In some embodiments, the psoriatic arthritis treatment comprises a therapeutically effective amount of a Clustered Regularly Interspaced Short Palindromic Repeats (CRISPR) gene-editing system, the system comprising: (i) a CRISPR Associated (Cas) protein; and (ii) at least one guide RNA targeting IL1R1. In some embodiments, the psoriatic arthritis treatment comprises a CRISPR gene-editing system targeting hIL1R1. In some embodiments, the psoriatic arthritis treatment comprises a CRISPR gene-editing system targeting cIL1R1. In some embodiments, the psoriatic arthritis treatment comprises a CRISP gene-editing system R targeting eIL1R1. In some embodiments, the psoriatic arthritis treatment comprises a CRISPR gene-editing system targeting fIL1R1.

In some embodiments, the CRISPR gene-editing system for the treatment of psoriatic arthritis comprises one or more sgRNAs targeting exon 1 of hIL1R1. In some embodiments, the CRISPR gene-editing system for the treatment of psoriatic arthritis comprises one or more sgRNAs targeting exon 2 of hIL1R1. In some embodiments, the CRISPR gene-editing system for the treatment of psoriatic arthritis comprises one or more sgRNAs targeting exon 3 of hIL1R1. In some embodiments, the CRISPR gene-editing system for the treatment of psoriatic arthritis comprises one or more sgRNAs targeting exon 4 of hIL1R1. In some embodiments, the CRISPR gene-editing system for the treatment of psoriatic arthritis comprises one or more sgRNAs targeting exon 5 of hIL1R1. In some embodiments, the CRISPR gene-editing system for the treatment of psoriatic arthritis comprises one or more sgRNAs targeting exon 6 of hIL1R1. In some embodiments, the CRISPR gene-editing system for the treatment of psoriatic arthritis comprises one or more sgRNAs targeting exon 7 of hIL1R1. In some embodiments, the CRISPR gene-editing system for the treatment of psoriatic arthritis comprises one or more sgRNAs targeting exon 8 of hIL1R1. In some embodiments, the CRISPR gene-editing system for the treatment of psoriatic arthritis comprises one or more sgRNAs targeting exon 9 of hIL1R1. In some embodiments, the CRISPR gene-editing system for the treatment of psoriatic arthritis comprises one or more sgRNAs targeting exon 10 of hIL1R1. In some embodiments, the CRISPR gene-editing system for the treatment of psoriatic arthritis comprises one or more sgRNAs targeting exon 11 of hIL1R1. In some embodiments, the CRISPR gene-editing system for the treatment of psoriatic arthritis comprises one or more sgRNAs targeting exon 12 of hIL1R1. In some embodiments, the CRISPR gene-editing system for the treatment of psoriatic arthritis comprises one or more sgRNAs targeting exon 13 of hIL1R1. In some embodiments, the CRISPR gene-editing system for the treatment of psoriatic arthritis comprises one or more sgRNAs targeting exon 14 of hIL1R1. In some embodiments, the CRISPR gene-editing system for the treatment of psoriatic arthritis comprises one or more sgRNAs targeting exon 15 of hIL1R1. In some embodiments, the CRISPR gene-editing system for the treatment of psoriatic arthritis comprises one or more sgRNAs targeting exon 16 of hIL1R1. In some embodiments, the CRISPR gene-editing system for the treatment of psoriatic arthritis comprises one or more sgRNAs targeting exon 17 of hIL1R1. In some embodiments, the CRISPR gene-editing system for the treatment of psoriatic arthritis comprises one or more sgRNAs targeting exon 18 of hIL1R1. In some embodiments, the CRISPR gene-editing system for the treatment of psoriatic arthritis comprises one or more sgRNAs targeting exon 19 of hIL1R1. In some embodiments, the CRISPR gene-editing system for the treatment of psoriatic arthritis comprises one or more sgRNAs targeting exon 20 of hIL1R1. In some embodiments, the CRISPR gene-editing system for the treatment of psoriatic arthritis comprises one or more sgRNAs targeting exon 21 of hIL1R1.

In some embodiments, the CRISPR gene-editing system for the treatment of psoriatic arthritis comprises one or more sgRNAs targeting exon 1 of cIL1R1. In some embodiments, the CRISPR gene-editing system for the treatment of psoriatic arthritis comprises one or more sgRNAs targeting exon 2 of cIL1R1. In some embodiments, the CRISPR gene-editing system for the treatment of psoriatic arthritis comprises one or more sgRNAs targeting exon 3 of cIL1R1. In some embodiments, the CRISPR gene-editing system for the treatment of psoriatic arthritis comprises one or more sgRNAs targeting exon 4 of cIL1R1. In some embodiments, the CRISPR gene-editing system for the treatment of psoriatic arthritis comprises one or more sgRNAs targeting exon 5 of cIL1R1. In some embodiments, the CRISPR gene-editing system for the treatment of psoriatic arthritis comprises one or more sgRNAs targeting exon 6 of cIL1R1. In some embodiments, the CRISPR gene-editing system for the treatment of psoriatic arthritis comprises one or more sgRNAs targeting exon 7 of cIL1R1. In some embodiments, the CRISPR gene-editing system for the treatment of psoriatic arthritis comprises one or more sgRNAs targeting exon 8 of cIL1R1. In some embodiments, the CRISPR gene-editing system for the treatment of psoriatic arthritis comprises one or more sgRNAs targeting exon 9 of cIL1R1. In some embodiments, the CRISPR gene-editing system for the treatment of psoriatic arthritis comprises one or more sgRNAs targeting exon 10 of cIL1R1. In some embodiments, the CRISPR gene-editing system for the treatment of psoriatic arthritis comprises one or more sgRNAs targeting exon 11 of cIL1R1. In some embodiments, the CRISPR gene-editing system for the treatment of psoriatic arthritis comprises one or more sgRNAs targeting exon 12 of cIL1R1. In some embodiments, the CRISPR gene-editing system for the treatment of psoriatic arthritis comprises one or more sgRNAs targeting exon 13 of cIL1R1. In some embodiments, the CRISPR gene-editing system for the treatment of psoriatic arthritis comprises one or more sgRNAs targeting exon 14 of cIL1R1. In some embodiments, the CRISPR gene-editing system for the treatment of psoriatic arthritis comprises one or more sgRNAs targeting exon 15 of cIL1R1. In some embodiments, the CRISPR gene-editing system for the treatment of psoriatic arthritis comprises one or more sgRNAs targeting exon 16 of cIL1R1.

In some embodiments, the CRISPR gene-editing system for the treatment of psoriatic arthritis comprises one or more sgRNAs targeting exon 1 of eIL1R1. In some embodiments, the CRISPR gene-editing system for the treatment of psoriatic arthritis comprises one or more sgRNAs targeting exon 2 of eIL1R1. In some embodiments, the CRISPR gene-editing system for the treatment of psoriatic arthritis comprises one or more sgRNAs targeting exon 3 of eIL1R1. In some embodiments, the CRISPR gene-editing system for the treatment of psoriatic arthritis comprises one or more sgRNAs targeting exon 4 of eIL1R1. In some embodiments, the CRISPR gene-editing system for the treatment of psoriatic arthritis comprises one or more sgRNAs targeting exon 5 of eIL1R1. In some embodiments, the CRISPR gene-editing system for the treatment of psoriatic arthritis comprises one or more sgRNAs targeting exon 6 of eIL1R1. In some embodiments, the CRISPR gene-editing system for the treatment of psoriatic arthritis comprises one or more sgRNAs targeting exon 7 of eIL1R1. In some embodiments, the CRISPR gene-editing system for the treatment of psoriatic arthritis comprises one or more sgRNAs targeting exon 8 of eIL1R1. In some embodiments, the CRISPR gene-editing system for the treatment of psoriatic arthritis comprises one or more sgRNAs targeting exon 9 of eIL1R1. In some embodiments, the CRISPR gene-editing system for the treatment of psoriatic arthritis comprises one or more sgRNAs targeting exon 10 of eIL1R1. In some embodiments, the CRISPR gene-editing system for the treatment of psoriatic arthritis comprises one or more sgRNAs targeting exon 11 of eIL1R1. In some embodiments, the CRISPR gene-editing system for the treatment of psoriatic arthritis comprises one or more sgRNAs targeting exon 12 of eIL1R1. In some embodiments, the CRISPR gene-editing system for the treatment of psoriatic arthritis comprises one or more sgRNAs targeting exon 13 of eIL1R1. In some embodiments, the CRISPR gene-editing system for the treatment of psoriatic arthritis comprises one or more sgRNAs targeting exon 14 of eIL1R1. In some embodiments, the CRISPR gene-editing system for the treatment of psoriatic arthritis comprises one or more sgRNAs targeting exon 15 of eIL1R1.

In some embodiments, the CRISPR gene-editing system for the treatment of psoriatic arthritis comprises one or more sgRNAs targeting exon 1 of fIL1R1. In some embodiments, the CRISPR gene-editing system for the treatment of psoriatic arthritis comprises one or more sgRNAs targeting exon 2 of fIL1R1. In some embodiments, the CRISPR gene-editing system for the treatment of psoriatic arthritis comprises one or more sgRNAs targeting exon 3 of fIL1R1. In some embodiments, the CRISPR gene-editing system for the treatment of psoriatic arthritis comprises one or more sgRNAs targeting exon 4 of fIL1R1. In some embodiments, the CRISPR gene-editing system for the treatment of psoriatic arthritis comprises one or more sgRNAs targeting exon 5 of fIL1R1. In some embodiments, the CRISPR gene-editing system for the treatment of psoriatic arthritis comprises one or more sgRNAs targeting exon 6 of fIL1R1. In some embodiments, the CRISPR gene-editing system for the treatment of psoriatic arthritis comprises one or more sgRNAs targeting exon 7 of fIL1R1. In some embodiments, the CRISPR gene-editing system for the treatment of psoriatic arthritis comprises one or more sgRNAs targeting exon 8 of fIL1R1. In some embodiments, the CRISPR gene-editing system for the treatment of psoriatic arthritis comprises one or more sgRNAs targeting exon 9 of fIL1R1. In some embodiments, the CRISPR gene-editing system for the treatment of psoriatic arthritis comprises one or more sgRNAs targeting exon 10 of fIL1R1. In some embodiments, the CRISPR gene-editing system for the treatment of psoriatic arthritis comprises one or more sgRNAs targeting exon 11 of fIL1R1. In some embodiments, the CRISPR gene-editing system for the treatment of psoriatic arthritis comprises one or more sgRNAs targeting exon 12 of fIL1R1. In some embodiments, the CRISPR gene-editing system for the treatment of psoriatic arthritis comprises one or more sgRNAs targeting exon 13 of fIL1R1. In some embodiments, the CRISPR gene-editing system for the treatment of psoriatic arthritis comprises one or more sgRNAs targeting exon 14 of fIL1R1. In some embodiments, the CRISPR gene-editing system for the treatment of psoriatic arthritis comprises one or more sgRNAs targeting exon 15 of fIL1R1. In some embodiments, the CRISPR gene-editing system for the treatment of psoriatic arthritis comprises one or more sgRNAs targeting exon 16 of fIL1R1. In some embodiments, the CRISPR gene-editing system for the treatment of psoriatic arthritis comprises one or more sgRNAs targeting exon 17 of fIL1R1. In some embodiments, the CRISPR gene-editing system for the treatment of psoriatic arthritis comprises one or more sgRNAs targeting exon 18 of fIL1R1.

In some embodiments, the CRISPR gene-editing system for the treatment of psoriatic arthritis comprises one or more sgRNAs targeting IL1RAP. In some embodiments, the CRISPR gene-editing system for the treatment of psoriatic arthritis comprises one or more sgRNAs targeting hIL1RAP. In some embodiments, the CRISPR gene-editing system for the treatment of psoriatic arthritis comprises one or more sgRNAs targeting cIL1RAP. In some embodiments, the CRISPR gene-editing system for the treatment of psoriatic arthritis comprises one or more sgRNAs targeting eIL1RAP. In some embodiments, the CRISPR gene-editing system for the treatment of psoriatic arthritis comprises one or more sgRNAs targeting fIL1RAP.

In some embodiments, the CRISPR gene-editing system for the treatment of psoriatic arthritis comprises one or more sgRNAs targeting exon 1 of hIL1RAP. In some embodiments, the CRISPR gene-editing system for the treatment of psoriatic arthritis comprises one or more sgRNAs targeting exon 2 of hIL1RAP. In some embodiments, the CRISPR gene-editing system for the treatment of psoriatic arthritis comprises one or more sgRNAs targeting exon 3 of hIL1RAP. In some embodiments, the CRISPR gene-editing system for the treatment of psoriatic arthritis comprises one or more sgRNAs targeting exon 4 of hIL1RAP. In some embodiments, the CRISPR gene-editing system for the treatment of psoriatic arthritis comprises one or more sgRNAs targeting exon 5 of hIL1RAP. In some embodiments, the CRISPR gene-editing system for the treatment of psoriatic arthritis comprises one or more sgRNAs targeting exon 6 of hIL1RAP. In some embodiments, the CRISPR gene-editing system for the treatment of psoriatic arthritis comprises one or more sgRNAs targeting exon 7 of hIL1RAP. In some embodiments, the CRISPR gene-editing system for the treatment of psoriatic arthritis comprises one or more sgRNAs targeting exon 8 of hIL1RAP. In some embodiments, the CRISPR gene-editing system for the treatment of psoriatic arthritis comprises one or more sgRNAs targeting exon 9 of hIL1RAP. In some embodiments, the CRISPR gene-editing system for the treatment of psoriatic arthritis comprises one or more sgRNAs targeting exon 10 of hIL1RAP. In some embodiments, the CRISPR gene-editing system for the treatment of psoriatic arthritis comprises one or more sgRNAs targeting exon 11 of hIL1RAP. In some embodiments, the CRISPR gene-editing system for the treatment of psoriatic arthritis comprises one or more sgRNAs targeting exon 12 of hIL1RAP. In some embodiments, the CRISPR gene-editing system for the treatment of psoriatic arthritis comprises one or more sgRNAs targeting exon 13 of hIL1RAP. In some embodiments, the CRISPR gene-editing system for the treatment of psoriatic arthritis comprises one or more sgRNAs targeting exon 14 of hIL1RAP. In some embodiments, the CRISPR gene-editing system for the treatment of psoriatic arthritis comprises one or more sgRNAs targeting exon 15 of hIL1RAP. In some embodiments, the CRISPR gene-editing system for the treatment of psoriatic arthritis comprises one or more sgRNAs targeting exon 16 of hIL1RAP. In some embodiments, the CRISPR gene-editing system for the treatment of psoriatic arthritis comprises one or more sgRNAs targeting exon 17 of hIL1RAP.

In some embodiments, the CRISPR gene-editing system for the treatment of psoriatic arthritis comprises one or more sgRNAs targeting exon 1 of cIL1RAP. In some embodiments, the CRISPR gene-editing system for the treatment of psoriatic arthritis comprises one or more sgRNAs targeting exon 2 of cIL1RAP. In some embodiments, the CRISPR gene-editing system for the treatment of psoriatic arthritis comprises one or more sgRNAs targeting exon 3 of cIL1RAP. In some embodiments, the CRISPR gene-editing system for the treatment of psoriatic arthritis comprises one or more sgRNAs targeting exon 4 of cIL1RAP. In some embodiments, the CRISPR gene-editing system for the treatment of psoriatic arthritis comprises one or more sgRNAs targeting exon 5 of cIL1RAP. In some embodiments, the CRISPR gene-editing system for the treatment of psoriatic arthritis comprises one or more sgRNAs targeting exon 6 of cIL1RAP. In some embodiments, the CRISPR gene-editing system for the treatment of psoriatic arthritis comprises one or more sgRNAs targeting exon 7 of cIL1RAP. In some embodiments, the CRISPR gene-editing system for the treatment of psoriatic arthritis comprises one or more sgRNAs targeting exon 8 of cIL1RAP. In some embodiments, the CRISPR gene-editing system for the treatment of psoriatic arthritis comprises one or more sgRNAs targeting exon 9 of cIL1RAP. In some embodiments, the CRISPR gene-editing system for the treatment of psoriatic arthritis comprises one or more sgRNAs targeting exon 10 of cIL1RAP. In some embodiments, the CRISPR gene-editing system for the treatment of psoriatic arthritis comprises one or more sgRNAs targeting exon 11 of cIL1RAP. In some embodiments, the CRISPR gene-editing system for the treatment of psoriatic arthritis comprises one or more sgRNAs targeting exon 12 of cIL1RAP. In some embodiments, the CRISPR gene-editing system for the treatment of psoriatic arthritis comprises one or more sgRNAs targeting exon 13 of cIL1RAP. In some embodiments, the CRISPR gene-editing system for the treatment of psoriatic arthritis comprises one or more sgRNAs targeting exon 14 of cIL1RAP. In some embodiments, the CRISPR gene-editing system for the treatment of psoriatic arthritis comprises one or more sgRNAs targeting exon 15 of cIL1RAP. In some embodiments, the CRISPR gene-editing system for the treatment of psoriatic arthritis comprises one or more sgRNAs targeting exon 16 of cIL1RAP.

In some embodiments, the CRISPR gene-editing system for the treatment of psoriatic arthritis comprises one or more sgRNAs targeting exon 1 of eIL1RAP. In some embodiments, the CRISPR gene-editing system for the treatment of psoriatic arthritis comprises one or more sgRNAs targeting exon 2 of eIL1RAP. In some embodiments, the CRISPR gene-editing system for the treatment of psoriatic arthritis comprises one or more sgRNAs targeting exon 3 of eIL1RAP. In some embodiments, the CRISPR gene-editing system for the treatment of psoriatic arthritis comprises one or more sgRNAs targeting exon 4 of eIL1RAP. In some embodiments, the CRISPR gene-editing system for the treatment of psoriatic arthritis comprises one or more sgRNAs targeting exon 5 of eIL1RAP. In some embodiments, the CRISPR gene-editing system for the treatment of psoriatic arthritis comprises one or more sgRNAs targeting exon 6 of eIL1RAP. In some embodiments, the CRISPR gene-editing system for the treatment of psoriatic arthritis comprises one or more sgRNAs targeting exon 7 of eIL1RAP. In some embodiments, the CRISPR gene-editing system for the treatment of psoriatic arthritis comprises one or more sgRNAs targeting exon 8 of eIL1RAP. In some embodiments, the CRISPR gene-editing system for the treatment of psoriatic arthritis comprises one or more sgRNAs targeting exon 9 of eIL1RAP. In some embodiments, the CRISPR gene-editing system for the treatment of psoriatic arthritis comprises one or more sgRNAs targeting exon 10 of eIL1RAP. In some embodiments, the CRISPR gene-editing system for the treatment of psoriatic arthritis comprises one or more sgRNAs targeting exon 11 of eIL1RAP. In some embodiments, the CRISPR gene-editing system for the treatment of psoriatic arthritis comprises one or more sgRNAs targeting exon 12 of eIL1RAP. In some embodiments, the CRISPR gene-editing system for the treatment of psoriatic arthritis comprises one or more sgRNAs targeting exon 13 of eIL1RAP.

In some embodiments, the CRISPR gene-editing system for the treatment of psoriatic arthritis comprises one or more sgRNAs targeting exon 1 of fIL1RAP. In some embodiments, the CRISPR gene-editing system for the treatment of psoriatic arthritis comprises one or more sgRNAs targeting exon 2 of fIL1RAP. In some embodiments, the CRISPR gene-editing system for the treatment of psoriatic arthritis comprises one or more sgRNAs targeting exon 3 of fIL1RAP. In some embodiments, the CRISPR gene-editing system for the treatment of psoriatic arthritis comprises one or more sgRNAs targeting exon 4 of fIL1RAP. In some embodiments, the CRISPR gene-editing system for the treatment of psoriatic arthritis comprises one or more sgRNAs targeting exon 5 of fIL1RAP. In some embodiments, the CRISPR gene-editing system for the treatment of psoriatic arthritis comprises one or more sgRNAs targeting exon 6 of fIL1RAP. In some embodiments, the CRISPR gene-editing system for the treatment of psoriatic arthritis comprises one or more sgRNAs targeting exon 7 of fIL1RAP. In some embodiments, the CRISPR gene-editing system for the treatment of psoriatic arthritis comprises one or more sgRNAs targeting exon 8 of fIL1RAP. In some embodiments, the CRISPR gene-editing system for the treatment of psoriatic arthritis comprises one or more sgRNAs targeting exon 9 of fIL1RAP. In some embodiments, the CRISPR gene-editing system for the treatment of psoriatic arthritis comprises one or more sgRNAs targeting exon 10 of fIL1RAP. In some embodiments, the CRISPR gene-editing system for the treatment of psoriatic arthritis comprises one or more sgRNAs targeting exon 11 of fIL1RAP. In some embodiments, the CRISPR gene-editing system for the treatment of psoriatic arthritis comprises one or more sgRNAs targeting exon 12 of fIL1RAP. In some embodiments, the CRISPR gene-editing system for the treatment of psoriatic arthritis comprises one or more sgRNAs targeting exon 13 of fIL1RAP. In some embodiments, the CRISPR gene-editing system for the treatment of psoriatic arthritis comprises one or more sgRNAs targeting exon 14 of fIL1RAP.

In some embodiments, the CRISPR gene-editing system for the treatment of psoriatic arthritis comprises one or more sgRNAs targeting TGFBR1. In some embodiments, the CRISPR gene-editing system for the treatment of psoriatic arthritis comprises one or more sgRNAs targeting hTGFBR1. In some embodiments, the CRISPR gene-editing system for the treatment of psoriatic arthritis comprises one or more sgRNAs targeting cTGFBR1. In some embodiments, the CRISPR gene-editing system for the treatment of psoriatic arthritis comprises one or more sgRNAs targeting e TGFBR1. In some embodiments, the CRISPR gene-editing system for the treatment of psoriatic arthritis comprises one or more sgRNAs targeting fTGFBR1.

In some embodiments, the CRISPR gene-editing system for the treatment of psoriatic arthritis comprises one or more sgRNAs targeting exon 1 of hTGFBR1. In some embodiments, the CRISPR gene-editing system for the treatment of psoriatic arthritis comprises one or more sgRNAs targeting exon 2 of hTGFBR1. In some embodiments, the CRISPR gene-editing system for the treatment of psoriatic arthritis comprises one or more sgRNAs targeting exon 3 of hTGFBR1. In some embodiments, the CRISPR gene-editing system for the treatment of psoriatic arthritis comprises one or more sgRNAs targeting exon 4 of hTGFBR1. In some embodiments, the CRISPR gene-editing system for the treatment of psoriatic arthritis comprises one or more sgRNAs targeting exon 5 of hTGFBR1. In some embodiments, the CRISPR gene-editing system for the treatment of psoriatic arthritis comprises one or more sgRNAs targeting exon 6 of hTGFBR1. In some embodiments, the CRISPR gene-editing system for the treatment of psoriatic arthritis comprises one or more sgRNAs targeting exon 7 of hTGFBR1. In some embodiments, the CRISPR gene-editing system for the treatment of psoriatic arthritis comprises one or more sgRNAs targeting exon 8 of hTGFBR1. In some embodiments, the CRISPR gene-editing system for the treatment of psoriatic arthritis comprises one or more sgRNAs targeting exon 9 of hTGFBR1. In some embodiments, the CRISPR gene-editing system for the treatment of psoriatic arthritis comprises one or more sgRNAs targeting exon 10 of hTGFBR1. In some embodiments, the CRISPR gene-editing system for the treatment of psoriatic arthritis comprises one or more sgRNAs targeting exon 11 of hTGFBR1.

In some embodiments, the CRISPR gene-editing system for the treatment of psoriatic arthritis comprises one or more sgRNAs targeting exon 1 of cTGFBR1. In some embodiments, the CRISPR gene-editing system for the treatment of psoriatic arthritis comprises one or more sgRNAs targeting exon 2 of cTGFBR1. In some embodiments, the CRISPR gene-editing system for the treatment of psoriatic arthritis comprises one or more sgRNAs targeting exon 3 of cTGFBR1. In some embodiments, the CRISPR gene-editing system for the treatment of psoriatic arthritis comprises one or more sgRNAs targeting exon 4 of cTGFBR1. In some embodiments, the CRISPR gene-editing system for the treatment of psoriatic arthritis comprises one or more sgRNAs targeting exon 5 of cTGFBR1. In some embodiments, the CRISPR gene-editing system for the treatment of psoriatic arthritis comprises one or more sgRNAs targeting exon 6 of cTGFBR1. In some embodiments, the CRISPR gene-editing system for the treatment of psoriatic arthritis comprises one or more sgRNAs targeting exon 7 of cTGFBR1. In some embodiments, the CRISPR gene-editing system for the treatment of psoriatic arthritis comprises one or more sgRNAs targeting exon 8 of cTGFBR1. In some embodiments, the CRISPR gene-editing system for the treatment of psoriatic arthritis comprises one or more sgRNAs targeting exon 9 of cTGFBR1.

In some embodiments, the CRISPR gene-editing system for the treatment of psoriatic arthritis comprises one or more sgRNAs targeting exon 1 of eTGFBR1. In some embodiments, the CRISPR gene-editing system for the treatment of psoriatic arthritis comprises one or more sgRNAs targeting exon 2 of eTGFBR1. In some embodiments, the CRISPR gene-editing system for the treatment of psoriatic arthritis comprises one or more sgRNAs targeting exon 3 of eTGFBR1. In some embodiments, the CRISPR gene-editing system for the treatment of psoriatic arthritis comprises one or more sgRNAs targeting exon 4 of eTGFBR1. In some embodiments, the CRISPR gene-editing system for the treatment of psoriatic arthritis comprises one or more sgRNAs targeting exon 5 of eTGFBR1. In some embodiments, the CRISPR gene-editing system for the treatment of psoriatic arthritis comprises one or more sgRNAs targeting exon 6 of eTGFBR1. In some embodiments, the CRISPR gene-editing system for the treatment of psoriatic arthritis comprises one or more sgRNAs targeting exon 7 of eTGFBR1. In some embodiments, the CRISPR gene-editing system for the treatment of psoriatic arthritis comprises one or more sgRNAs targeting exon 8 of eTGFBR1. In some embodiments, the CRISPR gene-editing system for the treatment of psoriatic arthritis comprises one or more sgRNAs targeting exon 9 of eTGFBR1. In some embodiments, the CRISPR gene-editing system for the treatment of psoriatic arthritis comprises one or more sgRNAs targeting exon 10 of eTGFBR1. In some embodiments, the CRISPR gene-editing system for the treatment of psoriatic arthritis comprises one or more sgRNAs targeting exon 11 of eTGFBR1. In some embodiments, the CRISPR gene-editing system for the treatment of psoriatic arthritis comprises one or more sgRNAs targeting exon 12 of eTGFBR1.

In some embodiments, the CRISPR gene-editing system for the treatment of psoriatic arthritis comprises one or more sgRNAs targeting exon 1 of fTGFBR1. In some embodiments, the CRISPR gene-editing system for the treatment of psoriatic arthritis comprises one or more sgRNAs targeting exon 2 of fTGFBR1. In some embodiments, the CRISPR gene-editing system for the treatment of psoriatic arthritis comprises one or more sgRNAs targeting exon 3 of fTGFBR1. In some embodiments, the CRISPR gene-editing system for the treatment of psoriatic arthritis comprises one or more sgRNAs targeting exon 4 of fTGFBR1. In some embodiments, the CRISPR gene-editing system for the treatment of psoriatic arthritis comprises one or more sgRNAs targeting exon 5 of fTGFBR1. In some embodiments, the CRISPR gene-editing system for the treatment of psoriatic arthritis comprises one or more sgRNAs targeting exon 6 of fTGFBR1. In some embodiments, the CRISPR gene-editing system for the treatment of psoriatic arthritis comprises one or more sgRNAs targeting exon 7 of fTGFBR1. In some embodiments, the CRISPR gene-editing system for the treatment of psoriatic arthritis comprises one or more sgRNAs targeting exon 8 of fTGFBR1. In some embodiments, the CRISPR gene-editing system for the treatment of psoriatic arthritis comprises one or more sgRNAs targeting exon 9 of fTGFBR1. In some embodiments, the CRISPR gene-editing system for the treatment of psoriatic arthritis comprises one or more sgRNAs targeting exon 10 of fTGFBR1. In some embodiments, the CRISPR gene-editing system for the treatment of psoriatic arthritis comprises one or more sgRNAs targeting exon 11 of fTGFBR1.

In some embodiments, the CRISPR gene-editing system for the treatment of psoriatic arthritis comprises one or more sgRNAs targeting TGFBR2. In some embodiments, the CRISPR gene-editing system for the treatment of psoriatic arthritis comprises one or more sgRNAs targeting hTGFBR2. In some embodiments, the CRISPR gene-editing system for the treatment of psoriatic arthritis comprises one or more sgRNAs targeting cTGFBR2. In some embodiments, the CRISPR gene-editing system for the treatment of psoriatic arthritis comprises one or more sgRNAs targeting eTGFBR2. In some embodiments, the CRISPR gene-editing system for the treatment of psoriatic arthritis comprises one or more sgRNAs targeting fTGFBR2.

In some embodiments, the CRISPR gene-editing system for the treatment of psoriatic arthritis comprises one or more sgRNAs targeting exon 1 of hTGFBR2. In some embodiments, the CRISPR gene-editing system for the treatment of psoriatic arthritis comprises one or more sgRNAs targeting exon 2 of hTGFBR2. In some embodiments, the CRISPR gene-editing system for the treatment of psoriatic arthritis comprises one or more sgRNAs targeting exon 3 of hTGFBR2. In some embodiments, the CRISPR gene-editing system for the treatment of psoriatic arthritis comprises one or more sgRNAs targeting exon 4 of hTGFBR2. In some embodiments, the CRISPR gene-editing system for the treatment of psoriatic arthritis comprises one or more sgRNAs targeting exon 5 of hTGFBR2. In some embodiments, the CRISPR gene-editing system for the treatment of psoriatic arthritis comprises one or more sgRNAs targeting exon 6 of hTGFBR2. In some embodiments, the CRISPR gene-editing system for the treatment of psoriatic arthritis comprises one or more sgRNAs targeting exon 7 of hTGFBR2. In some embodiments, the CRISPR gene-editing system for the treatment of psoriatic arthritis comprises one or more sgRNAs targeting exon 8 of hTGFBR2. In some embodiments, the CRISPR gene-editing system for the treatment of psoriatic arthritis comprises one or more sgRNAs targeting exon 9 of hTGFBR2.

In some embodiments, the CRISPR gene-editing system for the treatment of psoriatic arthritis comprises one or more sgRNAs targeting exon 1 of cTGFBR2. In some embodiments, the CRISPR gene-editing system for the treatment of psoriatic arthritis comprises one or more sgRNAs targeting exon 2 of cTGFBR2. In some embodiments, the CRISPR gene-editing system for the treatment of psoriatic arthritis comprises one or more sgRNAs targeting exon 3 of cTGFBR2. In some embodiments, the CRISPR gene-editing system for the treatment of psoriatic arthritis comprises one or more sgRNAs targeting exon 4 of cTGFBR2. In some embodiments, the CRISPR gene-editing system for the treatment of psoriatic arthritis comprises one or more sgRNAs targeting exon 5 of cTGFBR2. In some embodiments, the CRISPR gene-editing system for the treatment of psoriatic arthritis comprises one or more sgRNAs targeting exon 6 of cTGFBR2. In some embodiments, the CRISPR gene-editing system for the treatment of psoriatic arthritis comprises one or more sgRNAs targeting exon 7 of cTGFBR2. In some embodiments, the CRISPR gene-editing system for the treatment of psoriatic arthritis comprises one or more sgRNAs targeting exon 8 of cTGFBR2.

In some embodiments, the CRISPR gene-editing system for the treatment of psoriatic arthritis comprises one or more sgRNAs targeting exon 1 of eTGFBR2. In some embodiments, the CRISPR gene-editing system for the treatment of psoriatic arthritis comprises one or more sgRNAs targeting exon 2 of eTGFBR2. In some embodiments, the CRISPR gene-editing system for the treatment of psoriatic arthritis comprises one or more sgRNAs targeting exon 3 of eTGFBR2. In some embodiments, the CRISPR gene-editing system for the treatment of psoriatic arthritis comprises one or more sgRNAs targeting exon 4 of eTGFBR2. In some embodiments, the CRISPR gene-editing system for the treatment of psoriatic arthritis comprises one or more sgRNAs targeting exon 5 of eTGFBR2. In some embodiments, the CRISPR gene-editing system for the treatment of psoriatic arthritis comprises one or more sgRNAs targeting exon 6 of eTGFBR2. In some embodiments, the CRISPR gene-editing system for the treatment of psoriatic arthritis comprises one or more sgRNAs targeting exon 7 of eTGFBR2. In some embodiments, the CRISPR gene-editing system for the treatment of psoriatic arthritis comprises one or more sgRNAs targeting exon 8 of eTGFBR2. In some embodiments, the CRISPR gene-editing system for the treatment of psoriatic arthritis comprises one or more sgRNAs targeting exon 9 of eTGFBR2. In some embodiments, the CRISPR gene-editing system for the treatment of psoriatic arthritis comprises one or more sgRNAs targeting exon 10 of eTGFBR2.

In some embodiments, the CRISPR gene-editing system for the treatment of psoriatic arthritis comprises one or more sgRNAs targeting exon 1 of fTGFBR2. In some embodiments, the CRISPR gene-editing system for the treatment of psoriatic arthritis comprises one or more sgRNAs targeting exon 2 of fTGFBR2. In some embodiments, the CRISPR gene-editing system for the treatment of psoriatic arthritis comprises one or more sgRNAs targeting exon 3 of fTGFBR2. In some embodiments, the CRISPR gene-editing system for the treatment of psoriatic arthritis comprises one or more sgRNAs targeting exon 4 of fTGFBR2. In some embodiments, the CRISPR gene-editing system for the treatment of psoriatic arthritis comprises one or more sgRNAs targeting exon 5 of fTGFBR2. In some embodiments, the CRISPR gene-editing system for the treatment of psoriatic arthritis comprises one or more sgRNAs targeting exon 6 of fTGFBR2. In some embodiments, the CRISPR gene-editing system for the treatment of psoriatic arthritis comprises one or more sgRNAs targeting exon 7 of fTGFBR2. In some embodiments, the CRISPR gene-editing system for the treatment of psoriatic arthritis comprises one or more sgRNAs targeting exon 8 of fTGFBR2.

In some embodiments, the CRISPR gene-editing system for the treatment of psoriatic arthritis comprises one or more sgRNAs targeting IL6R. In some embodiments, the CRISPR gene-editing system for the treatment of psoriatic arthritis comprises one or more sgRNAs targeting hIL6R. In some embodiments, the CRISPR gene-editing system for the treatment of psoriatic arthritis comprises one or more sgRNAs targeting cIL6R. In some embodiments, the CRISPR gene-editing system for the treatment of psoriatic arthritis comprises one or more sgRNAs targeting eIL6R. In some embodiments, the CRISPR gene-editing system for the treatment of psoriatic arthritis comprises one or more sgRNAs targeting fIL6R.

In some embodiments, the CRISPR gene-editing system for the treatment of psoriatic arthritis comprises one or more sgRNAs targeting exon 1 of hIL6R. In some embodiments, the CRISPR gene-editing system for the treatment of psoriatic arthritis comprises one or more sgRNAs targeting exon 2 of hIL6R. In some embodiments, the CRISPR gene-editing system for the treatment of psoriatic arthritis comprises one or more sgRNAs targeting exon 3 of hIL6R. In some embodiments, the CRISPR gene-editing system for the treatment of psoriatic arthritis comprises one or more sgRNAs targeting exon 4 of hIL6R. In some embodiments, the CRISPR gene-editing system for the treatment of psoriatic arthritis comprises one or more sgRNAs targeting exon 5 of hIL6R. In some embodiments, the CRISPR gene-editing system for the treatment of psoriatic arthritis comprises one or more sgRNAs targeting exon 6 of hIL6R. In some embodiments, the CRISPR gene-editing system for the treatment of psoriatic arthritis comprises one or more sgRNAs targeting exon 7 of hIL6R. In some embodiments, the CRISPR gene-editing system for the treatment of psoriatic arthritis comprises one or more sgRNAs targeting exon 8 of hIL6R. In some embodiments, the CRISPR gene-editing system for the treatment of psoriatic arthritis comprises one or more sgRNAs targeting exon 9 of hIL6R. In some embodiments, the CRISPR gene-editing system for the treatment of psoriatic arthritis comprises one or more sgRNAs targeting exon 10 of hIL6R. In some embodiments, the CRISPR gene-editing system for the treatment of psoriatic arthritis comprises one or more sgRNAs targeting exon 11 of hIL6R In some embodiments, the CRISPR gene-editing system for the treatment of psoriatic arthritis comprises one or more sgRNAs targeting exon 12 of hIL6R. In some embodiments, the CRISPR gene-editing system for the treatment of psoriatic arthritis comprises one or more sgRNAs targeting exon 13 of hIL6R. In some embodiments, the CRISPR gene-editing system for the treatment of psoriatic arthritis comprises one or more sgRNAs targeting exon 14 of hIL6R. In some embodiments, the CRISPR gene-editing system for the treatment of psoriatic arthritis comprises one or more sgRNAs targeting exon 15 of hIL6R.

In some embodiments, the CRISPR gene-editing system for the treatment of psoriatic arthritis comprises one or more sgRNAs targeting exon 1 of cIL6R. In some embodiments, the CRISPR gene-editing system for the treatment of psoriatic arthritis comprises one or more sgRNAs targeting exon 2 of cIL6R. In some embodiments, the CRISPR gene-editing system for the treatment of psoriatic arthritis comprises one or more sgRNAs targeting exon 3 of cIL6R. In some embodiments, the CRISPR gene-editing system for the treatment of psoriatic arthritis comprises one or more sgRNAs targeting exon 4 of cIL6R. In some embodiments, the CRISPR gene-editing system for the treatment of psoriatic arthritis comprises one or more sgRNAs targeting exon 5 of cIL6R. In some embodiments, the CRISPR gene-editing system for the treatment of psoriatic arthritis comprises one or more sgRNAs targeting exon 6 of cIL6R. In some embodiments, the CRISPR gene-editing system for the treatment of psoriatic arthritis comprises one or more sgRNAs targeting exon 7 of cIL6R. In some embodiments, the CRISPR gene-editing system for the treatment of psoriatic arthritis comprises one or more sgRNAs targeting exon 8 of cIL6R. In some embodiments, the CRISPR gene-editing system for the treatment of psoriatic arthritis comprises one or more sgRNAs targeting exon 9 of cIL6R. In some embodiments, the CRISPR gene-editing system for the treatment of psoriatic arthritis comprises one or more sgRNAs targeting exon 10 of cIL6R. In some embodiments, the CRISPR gene-editing system for the treatment of psoriatic arthritis comprises one or more sgRNAs targeting exon 11 of cIL6R. In some embodiments, the CRISPR gene-editing system for the treatment of psoriatic arthritis comprises one or more sgRNAs targeting exon 12 of cIL6R. In some embodiments, the CRISPR gene-editing system for the treatment of psoriatic arthritis comprises one or more sgRNAs targeting exon 13 of cIL6R.

In some embodiments, the CRISPR gene-editing system for the treatment of psoriatic arthritis comprises one or more sgRNAs targeting exon 1 of eIL6R. In some embodiments, the CRISPR gene-editing system for the treatment of psoriatic arthritis comprises one or more sgRNAs targeting exon 2 of eIL6R. In some embodiments, the CRISPR gene-editing system for the treatment of psoriatic arthritis comprises one or more sgRNAs targeting exon 3 of eIL6R. In some embodiments, the CRISPR gene-editing system for the treatment of psoriatic arthritis comprises one or more sgRNAs targeting exon 4 of eIL6R. In some embodiments, the CRISPR gene-editing system for the treatment of psoriatic arthritis comprises one or more sgRNAs targeting exon 5 of eIL6R. In some embodiments, the CRISPR gene-editing system for the treatment of psoriatic arthritis comprises one or more sgRNAs targeting exon 6 of eIL6R. In some embodiments, the CRISPR gene-editing system for the treatment of psoriatic arthritis comprises one or more sgRNAs targeting exon 7 of eIL6R. In some embodiments, the CRISPR gene-editing system for the treatment of psoriatic arthritis comprises one or more sgRNAs targeting exon 8 of eIL6R. In some embodiments, the CRISPR gene-editing system for the treatment of psoriatic arthritis comprises one or more sgRNAs targeting exon 9 of eIL6R. In some embodiments, the CRISPR gene-editing system for the treatment of psoriatic arthritis comprises one or more sgRNAs targeting exon 10 of eIL6R. In some embodiments, the CRISPR gene-editing system for the treatment of psoriatic arthritis comprises one or more sgRNAs targeting exon 11 of eIL6R.

In some embodiments, the CRISPR gene-editing system for the treatment of psoriatic arthritis comprises one or more sgRNAs targeting exon 1 of fIL6R. In some embodiments, the CRISPR gene-editing system for the treatment of psoriatic arthritis comprises one or more sgRNAs targeting exon 2 of fIL6R. In some embodiments, the CRISPR gene-editing system for the treatment of psoriatic arthritis comprises one or more sgRNAs targeting exon 3 of fIL6R. In some embodiments, the CRISPR gene-editing system for the treatment of psoriatic arthritis comprises one or more sgRNAs targeting exon 4 of fIL6R. In some embodiments, the CRISPR gene-editing system for the treatment of psoriatic arthritis comprises one or more sgRNAs targeting exon 5 of fIL6R. In some embodiments, the CRISPR gene-editing system for the treatment of psoriatic arthritis comprises one or more sgRNAs targeting exon 6 of fIL6R. In some embodiments, the CRISPR gene-editing system for the treatment of psoriatic arthritis comprises one or more sgRNAs targeting exon 7 of fIL6R. In some embodiments, the CRISPR gene-editing system for the treatment of psoriatic arthritis comprises one or more sgRNAs targeting exon 8 of fIL6R. In some embodiments, the CRISPR gene-editing system for the treatment of psoriatic arthritis comprises one or more sgRNAs targeting exon 9 of fIL6R. In some embodiments, the CRISPR gene-editing system for the treatment of psoriatic arthritis comprises one or more sgRNAs targeting exon 10 of fIL6R.

In some embodiments, the CRISPR gene-editing system for the treatment of psoriatic arthritis comprises one or more sgRNAs targeting IL6ST. In some embodiments, the CRISPR gene-editing system for the treatment of psoriatic arthritis comprises one or more sgRNAs targeting hIL6ST. In some embodiments, the CRISPR gene-editing system for the treatment of psoriatic arthritis comprises one or more sgRNAs targeting cIL6ST. In some embodiments, the CRISPR gene-editing system for the treatment of psoriatic arthritis comprises one or more sgRNAs targeting eIL6ST. In some embodiments, the CRISPR gene-editing system for the treatment of psoriatic arthritis comprises one or more sgRNAs targeting fIL6ST.

In some embodiments, the CRISPR gene-editing system for the treatment of psoriatic arthritis comprises one or more sgRNAs targeting exon 1 of hIL6ST. In some embodiments, the CRISPR gene-editing system for the treatment of psoriatic arthritis comprises one or more sgRNAs targeting exon 2 of hIL6ST. In some embodiments, the CRISPR gene-editing system for the treatment of psoriatic arthritis comprises one or more sgRNAs targeting exon 3 of hIL6ST. In some embodiments, the CRISPR gene-editing system for the treatment of psoriatic arthritis comprises one or more sgRNAs targeting exon 4 of hIL6ST. In some embodiments, the CRISPR gene-editing system for the treatment of psoriatic arthritis comprises one or more sgRNAs targeting exon 5 of hIL6ST. In some embodiments, the CRISPR gene-editing system for the treatment of psoriatic arthritis comprises one or more sgRNAs targeting exon 6 of hIL6ST. In some embodiments, the CRISPR gene-editing system for the treatment of psoriatic arthritis comprises one or more sgRNAs targeting exon 7 of hIL6ST. In some embodiments, the CRISPR gene-editing system for the treatment of psoriatic arthritis comprises one or more sgRNAs targeting exon 8 of hIL6ST. In some embodiments, the CRISPR gene-editing system for the treatment of psoriatic arthritis comprises one or more sgRNAs targeting exon 9 of hIL6ST. In some embodiments, the CRISPR gene-editing system for the treatment of psoriatic arthritis comprises one or more sgRNAs targeting exon 10 of hIL6ST. In some embodiments, the CRISPR gene-editing system for the treatment of psoriatic arthritis comprises one or more sgRNAs targeting exon 11 of hIL6ST. In some embodiments, the CRISPR gene-editing system for the treatment of psoriatic arthritis comprises one or more sgRNAs targeting exon 12 of hIL6ST. In some embodiments, the CRISPR gene-editing system for the treatment of psoriatic arthritis comprises one or more sgRNAs targeting exon 13 of hIL6ST. In some embodiments, the CRISPR gene-editing system for the treatment of psoriatic arthritis comprises one or more sgRNAs targeting exon 14 of hIL6ST. In some embodiments, the CRISPR gene-editing system for the treatment of psoriatic arthritis comprises one or more sgRNAs targeting exon 15 of hIL6ST. In some embodiments, the CRISPR gene-editing system for the treatment of psoriatic arthritis comprises one or more sgRNAs targeting exon 16 of hIL6ST. In some embodiments, the CRISPR gene-editing system for the treatment of psoriatic arthritis comprises one or more sgRNAs targeting exon 17 of hIL6ST. In some embodiments, the CRISPR gene-editing system for the treatment of psoriatic arthritis comprises one or more sgRNAs targeting exon 18 of hIL6ST. In some embodiments, the CRISPR gene-editing system for the treatment of psoriatic arthritis comprises one or more sgRNAs targeting exon 19 of hIL6ST.

In some embodiments, the CRISPR gene-editing system for the treatment of psoriatic arthritis comprises one or more sgRNAs targeting exon 1 of cIL6ST. In some embodiments, the CRISPR gene-editing system for the treatment of psoriatic arthritis comprises one or more sgRNAs targeting exon 2 of cIL6ST. In some embodiments, the CRISPR gene-editing system for the treatment of psoriatic arthritis comprises one or more sgRNAs targeting exon 3 of cIL6ST. In some embodiments, the CRISPR gene-editing system for the treatment of psoriatic arthritis comprises one or more sgRNAs targeting exon 4 of cIL6ST. In some embodiments, the CRISPR gene-editing system for the treatment of psoriatic arthritis comprises one or more sgRNAs targeting exon 5 of cIL6ST. In some embodiments, the CRISPR gene-editing system for the treatment of psoriatic arthritis comprises one or more sgRNAs targeting exon 6 of cIL6ST. In some embodiments, the CRISPR gene-editing system for the treatment of psoriatic arthritis comprises one or more sgRNAs targeting exon 7 of cIL6ST. In some embodiments, the CRISPR gene-editing system for the treatment of psoriatic arthritis comprises one or more sgRNAs targeting exon 8 of cIL6ST. In some embodiments, the CRISPR gene-editing system for the treatment of psoriatic arthritis comprises one or more sgRNAs targeting exon 9 of cIL6ST. In some embodiments, the CRISPR gene-editing system for the treatment of psoriatic arthritis comprises one or more sgRNAs targeting exon 10 of cIL6ST. In some embodiments, the CRISPR gene-editing system for the treatment of psoriatic arthritis comprises one or more sgRNAs targeting exon 11 of cIL6ST In some embodiments, the CRISPR gene-editing system for the treatment of psoriatic arthritis comprises one or more sgRNAs targeting exon 12 of cIL6ST. In some embodiments, the CRISPR gene-editing system for the treatment of psoriatic arthritis comprises one or more sgRNAs targeting exon 13 of cIL6ST. In some embodiments, the CRISPR gene-editing system for the treatment of psoriatic arthritis comprises one or more sgRNAs targeting exon 14 of cIL6ST. In some embodiments, the CRISPR gene-editing system for the treatment of psoriatic arthritis comprises one or more sgRNAs targeting exon 15 of cIL6ST. In some embodiments, the CRISPR gene-editing system for the treatment of psoriatic arthritis comprises one or more sgRNAs targeting exon 16 of cIL6ST. In some embodiments, the CRISPR gene-editing system for the treatment of psoriatic arthritis comprises one or more sgRNAs targeting exon 17 of cIL6ST. In some embodiments, the CRISPR gene-editing system for the treatment of psoriatic arthritis comprises one or more sgRNAs targeting exon 18 of cIL6ST.

In some embodiments, the CRISPR gene-editing system for the treatment of psoriatic arthritis comprises one or more sgRNAs targeting exon 1 of eIL6ST. In some embodiments, the CRISPR gene-editing system for the treatment of psoriatic arthritis comprises one or more sgRNAs targeting exon 2 of eIL6ST. In some embodiments, the CRISPR gene-editing system for the treatment of psoriatic arthritis comprises one or more sgRNAs targeting exon 3 of eIL6ST. In some embodiments, the CRISPR gene-editing system for the treatment of psoriatic arthritis comprises one or more sgRNAs targeting exon 4 of eIL6ST. In some embodiments, the CRISPR gene-editing system for the treatment of psoriatic arthritis comprises one or more sgRNAs targeting exon 5 of eIL6ST. In some embodiments, the CRISPR gene-editing system for the treatment of psoriatic arthritis comprises one or more sgRNAs targeting exon 6 of eIL6ST. In some embodiments, the CRISPR gene-editing system for the treatment of psoriatic arthritis comprises one or more sgRNAs targeting exon 7 of eIL6ST. In some embodiments, the CRISPR gene-editing system for the treatment of psoriatic arthritis comprises one or more sgRNAs targeting exon 8 of eIL6ST. In some embodiments, the CRISPR gene-editing system for the treatment of psoriatic arthritis comprises one or more sgRNAs targeting exon 9 of eIL6ST. In some embodiments, the CRISPR gene-editing system for the treatment of psoriatic arthritis comprises one or more sgRNAs targeting exon 10 of eIL6ST. In some embodiments, the CRISPR gene-editing system for the treatment of psoriatic arthritis comprises one or more sgRNAs targeting exon 11 of eIL6ST In some embodiments, the CRISPR gene-editing system for the treatment of psoriatic arthritis comprises one or more sgRNAs targeting exon 12 of eIL6ST. In some embodiments, the CRISPR gene-editing system for the treatment of psoriatic arthritis comprises one or more sgRNAs targeting exon 13 of eIL6ST. In some embodiments, the CRISPR gene-editing system for the treatment of psoriatic arthritis comprises one or more sgRNAs targeting exon 14 of eIL6ST. In some embodiments, the CRISPR gene-editing system for the treatment of psoriatic arthritis comprises one or more sgRNAs targeting exon 15 of eIL6ST. In some embodiments, the CRISPR gene-editing system for the treatment of psoriatic arthritis comprises one or more sgRNAs targeting exon 16 of eIL6ST. In some embodiments, the CRISPR gene-editing system for the treatment of psoriatic arthritis comprises one or more sgRNAs targeting exon 17 of eIL6ST. In some embodiments, the CRISPR gene-editing system for the treatment of psoriatic arthritis comprises one or more sgRNAs targeting exon 18 of eIL6ST.

In some embodiments, the CRISPR gene-editing system for the treatment of psoriatic arthritis comprises one or more sgRNAs targeting exon 1 of fIL6ST. In some embodiments, the CRISPR gene-editing system for the treatment of psoriatic arthritis comprises one or more sgRNAs targeting exon 2 of fIL6ST. In some embodiments, the CRISPR gene-editing system for the treatment of psoriatic arthritis comprises one or more sgRNAs targeting exon 3 of fIL6ST. In some embodiments, the CRISPR gene-editing system for the treatment of psoriatic arthritis comprises one or more sgRNAs targeting exon 4 of fIL6ST. In some embodiments, the CRISPR gene-editing system for the treatment of psoriatic arthritis comprises one or more sgRNAs targeting exon 5 of fIL6ST. In some embodiments, the CRISPR gene-editing system for the treatment of psoriatic arthritis comprises one or more sgRNAs targeting exon 6 of fIL6ST. In some embodiments, the CRISPR gene-editing system for the treatment of psoriatic arthritis comprises one or more sgRNAs targeting exon 7 of fIL6ST. In some embodiments, the CRISPR gene-editing system for the treatment of psoriatic arthritis comprises one or more sgRNAs targeting exon 8 of fIL6ST. In some embodiments, the CRISPR gene-editing system for the treatment of psoriatic arthritis comprises one or more sgRNAs targeting exon 9 of fIL6ST. In some embodiments, the CRISPR gene-editing system for the treatment of psoriatic arthritis comprises one or more sgRNAs targeting exon 10 of fIL6ST. In some embodiments, the CRISPR gene-editing system for the treatment of psoriatic arthritis comprises one or more sgRNAs targeting exon 11 of fIL6ST In some embodiments, the CRISPR gene-editing system for the treatment of psoriatic arthritis comprises one or more sgRNAs targeting exon 12 of fIL6ST. In some embodiments, the CRISPR gene-editing system for the treatment of psoriatic arthritis comprises one or more sgRNAs targeting exon 13 of fIL6ST. In some embodiments, the CRISPR gene-editing system for the treatment of psoriatic arthritis comprises one or more sgRNAs targeting exon 14 of fIL6ST. In some embodiments, the CRISPR gene-editing system for the treatment of psoriatic arthritis comprises one or more sgRNAs targeting exon 15 of fIL6ST. In some embodiments, the CRISPR gene-editing system for the treatment of psoriatic arthritis comprises one or more sgRNAs targeting exon 16 of fIL6ST. In some embodiments, the CRISPR gene-editing system for the treatment of psoriatic arthritis comprises one or more sgRNAs targeting exon 17 of fIL6ST. In some embodiments, the CRISPR gene-editing system for the treatment of psoriatic arthritis comprises one or more sgRNAs targeting exon 18 of fIL6ST. In some embodiments, the CRISPR gene-editing system for the treatment of psoriatic arthritis comprises one or more sgRNAs targeting exon 19 of fIL6ST. In some embodiments, the CRISPR gene-editing system for the treatment of psoriatic arthritis comprises one or more sgRNAs targeting exon 20 of fIL6ST.

In some embodiments, the CRISPR gene-editing system for the treatment of psoriatic arthritis comprises one or more sgRNAs targeting TNFRSF1A. In some embodiments, the CRISPR gene-editing system for the treatment of psoriatic arthritis comprises one or more sgRNAs targeting hTNFRSF1A. In some embodiments, the CRISPR gene-editing system for the treatment of psoriatic arthritis comprises one or more sgRNAs targeting cTNFRSF1A. In some embodiments, the CRISPR gene-editing system for the treatment of psoriatic arthritis comprises one or more sgRNAs targeting eTNFRSF1A. In some embodiments, the CRISPR gene-editing system for the treatment of psoriatic arthritis comprises one or more sgRNAs targeting fTNFRSF1A.

In some embodiments, the CRISPR gene-editing system for the treatment of psoriatic arthritis comprises one or more sgRNAs targeting exon 1 of hTNFRSF1A. In some embodiments, the CRISPR gene-editing system for the treatment of psoriatic arthritis comprises one or more sgRNAs targeting exon 2 of hTNFRSF1A. In some embodiments, the CRISPR gene-editing system for the treatment of psoriatic arthritis comprises one or more sgRNAs targeting exon 3 of hTNFRSF1A. In some embodiments, the CRISPR gene-editing system for the treatment of psoriatic arthritis comprises one or more sgRNAs targeting exon 4 of hTNFRSF1A. In some embodiments, the CRISPR gene-editing system for the treatment of psoriatic arthritis comprises one or more sgRNAs targeting exon 5 of hTNFRSF1A. In some embodiments, the CRISPR gene-editing system for the treatment of psoriatic arthritis comprises one or more sgRNAs targeting exon 6 of hTNFRSF1A. In some embodiments, the CRISPR gene-editing system for the treatment of psoriatic arthritis comprises one or more sgRNAs targeting exon 7 of hTNFRSF1A. In some embodiments, the CRISPR gene-editing system for the treatment of psoriatic arthritis comprises one or more sgRNAs targeting exon 8 of hTNFRSF1A. In some embodiments, the CRISPR gene-editing system for the treatment of psoriatic arthritis comprises one or more sgRNAs targeting exon 9 of hTNFRSF1A. In some embodiments, the CRISPR gene-editing system for the treatment of psoriatic arthritis comprises one or more sgRNAs targeting exon 10 of hTNFRSF1A. In some embodiments, the CRISPR gene-editing system for the treatment of psoriatic arthritis comprises one or more sgRNAs targeting exon 11 of hTNFRSF1A.

In some embodiments, the CRISPR gene-editing system for the treatment of psoriatic arthritis comprises one or more sgRNAs targeting exon 1 of cTNFRSF1A. In some embodiments, the CRISPR gene-editing system for the treatment of psoriatic arthritis comprises one or more sgRNAs targeting exon 2 of cTNFRSF1A. In some embodiments, the CRISPR gene-editing system for the treatment of psoriatic arthritis comprises one or more sgRNAs targeting exon 3 of cINFRSF1A. In some embodiments, the CRISPR gene-editing system for the treatment of psoriatic arthritis comprises one or more sgRNAs targeting exon 4 of cTNFRSF1A. In some embodiments, the CRISPR gene-editing system for the treatment of psoriatic arthritis comprises one or more sgRNAs targeting exon 5 of cTNFRSF1A. In some embodiments, the CRISPR gene-editing system for the treatment of psoriatic arthritis comprises one or more sgRNAs targeting exon 6 of cTNFRSF1A. In some embodiments, the CRISPR gene-editing system for the treatment of psoriatic arthritis comprises one or more sgRNAs targeting exon 7 of cTNFRSF1A. In some embodiments, the CRISPR gene-editing system for the treatment of psoriatic arthritis comprises one or more sgRNAs targeting exon 8 of cTNFRSF1A. In some embodiments, the CRISPR gene-editing system for the treatment of psoriatic arthritis comprises one or more sgRNAs targeting exon 9 of cTNFRSF1A. In some embodiments, the CRISPR gene-editing system for the treatment of psoriatic arthritis comprises one or more sgRNAs targeting exon 10 of cTNFRSF1A. In some embodiments, the CRISPR gene-editing system for the treatment of psoriatic arthritis comprises one or more sgRNAs targeting exon 11 of cTNFRSF1A.

In some embodiments, the CRISPR gene-editing system for the treatment of psoriatic arthritis comprises one or more sgRNAs targeting exon 1 of eTNFRSF1A. In some embodiments, the CRISPR gene-editing system for the treatment of psoriatic arthritis comprises one or more sgRNAs targeting exon 2 of eTNFRSF1A. In some embodiments, the CRISPR gene-editing system for the treatment of psoriatic arthritis comprises one or more sgRNAs targeting exon 3 of eTNFRSF1A. In some embodiments, the CRISPR gene-editing system for the treatment of psoriatic arthritis comprises one or more sgRNAs targeting exon 4 of eTNFRSF1A. In some embodiments, the CRISPR gene-editing system for the treatment of psoriatic arthritis comprises one or more sgRNAs targeting exon 5 of eTNFRSF1A. In some embodiments, the CRISPR gene-editing system for the treatment of psoriatic arthritis comprises one or more sgRNAs targeting exon 6 of eTNFRSF1A. In some embodiments, the CRISPR gene-editing system for the treatment of psoriatic arthritis comprises one or more sgRNAs targeting exon 7 of eTNFRSF1A. In some embodiments, the CRISPR gene-editing system for the treatment of psoriatic arthritis comprises one or more sgRNAs targeting exon 8 of eTNFRSF1A. In some embodiments, the CRISPR gene-editing system for the treatment of psoriatic arthritis comprises one or more sgRNAs targeting exon 9 of eTNFRSF1A. In some embodiments, the CRISPR gene-editing system for the treatment of psoriatic arthritis comprises one or more sgRNAs targeting exon 10 of eTNFRSF1A.

In some embodiments, the CRISPR gene-editing system for the treatment of psoriatic arthritis comprises one or more sgRNAs targeting exon 1 of fTNFRSF1A. In some embodiments, the CRISPR gene-editing system for the treatment of psoriatic arthritis comprises one or more sgRNAs targeting exon 2 of fTNFRSF1A. In some embodiments, the CRISPR gene-editing system for the treatment of psoriatic arthritis comprises one or more sgRNAs targeting exon 3 of fTNFRSF1A. In some embodiments, the CRISPR gene-editing system for the treatment of psoriatic arthritis comprises one or more sgRNAs targeting exon 4 of fTNFRSF1A. In some embodiments, the CRISPR gene-editing system for the treatment of psoriatic arthritis comprises one or more sgRNAs targeting exon 5 of fINFRSF1A. In some embodiments, the CRISPR gene-editing system for the treatment of psoriatic arthritis comprises one or more sgRNAs targeting exon 6 of fTNFRSF1A. In some embodiments, the CRISPR gene-editing system for the treatment of psoriatic arthritis comprises one or more sgRNAs targeting exon 7 of fTNFRSF1A. In some embodiments, the CRISPR gene-editing system for the treatment of psoriatic arthritis comprises one or more sgRNAs targeting exon 8 of fTNFRSF1A. In some embodiments, the CRISPR gene-editing system for the treatment of psoriatic arthritis comprises one or more sgRNAs targeting exon 9 of fTNFRSF1A. In some embodiments, the CRISPR gene-editing system for the treatment of psoriatic arthritis comprises one or more sgRNAs targeting exon 10 of fTNFRSF1A.

In some embodiments, the CRISPR gene-editing system for the treatment of psoriatic arthritis comprises one or more sgRNAs targeting TNFRSF1B. In some embodiments, the CRISPR gene-editing system for the treatment of psoriatic arthritis comprises one or more sgRNAs targeting hTNFRSF1B. In some embodiments, the CRISPR gene-editing system for the treatment of psoriatic arthritis comprises one or more sgRNAs targeting cTNFRSF1B. In some embodiments, the CRISPR gene-editing system for the treatment of psoriatic arthritis comprises one or more sgRNAs targeting eTNFRSF1B. In some embodiments, the CRISPR gene-editing system for the treatment of psoriatic arthritis comprises one or more sgRNAs targeting fTNFRSF1B.

In some embodiments, the CRISPR gene-editing system for the treatment of psoriatic arthritis comprises one or more sgRNAs targeting exon 1 of hTNFRSF1B. In some embodiments, the CRISPR gene-editing system for the treatment of psoriatic arthritis comprises one or more sgRNAs targeting exon 2 of hTNFRSF1B. In some embodiments, the CRISPR gene-editing system for the treatment of psoriatic arthritis comprises one or more sgRNAs targeting exon 3 of hTNFRSF1B. In some embodiments, the CRISPR gene-editing system for the treatment of psoriatic arthritis comprises one or more sgRNAs targeting exon 4 of hTNFRSF1B. In some embodiments, the CRISPR gene-editing system for the treatment of psoriatic arthritis comprises one or more sgRNAs targeting exon 5 of hTNFRSF1B. In some embodiments, the CRISPR gene-editing system for the treatment of psoriatic arthritis comprises one or more sgRNAs targeting exon 6 of hTNFRSF1B. In some embodiments, the CRISPR gene-editing system for the treatment of psoriatic arthritis comprises one or more sgRNAs targeting exon 7 of hTNFRSF1B. In some embodiments, the CRISPR gene-editing system for the treatment of psoriatic arthritis comprises one or more sgRNAs targeting exon 8 of hTNFRSF1B. In some embodiments, the CRISPR gene-editing system for the treatment of psoriatic arthritis comprises one or more sgRNAs targeting exon 9 of hTNFRSF1B. In some embodiments, the CRISPR gene-editing system for the treatment of psoriatic arthritis comprises one or more sgRNAs targeting exon 10 of hTNFRSF1B. In some embodiments, the CRISPR gene-editing system for the treatment of psoriatic arthritis comprises one or more sgRNAs targeting exon 11 of hTNFRSF1B In some embodiments, the CRISPR gene-editing system for the treatment of psoriatic arthritis comprises one or more sgRNAs targeting exon 12 of hTNFRSF1B. In some embodiments, the CRISPR gene-editing system for the treatment of psoriatic arthritis comprises one or more sgRNAs targeting exon 13 of hTNFRSF1B.

In some embodiments, the CRISPR gene-editing system for the treatment of psoriatic arthritis comprises one or more sgRNAs targeting exon 1 of cTNFRSF1B. In some embodiments, the CRISPR gene-editing system for the treatment of psoriatic arthritis comprises one or more sgRNAs targeting exon 2 of cTNFRSF1B. In some embodiments, the CRISPR gene-editing system for the treatment of psoriatic arthritis comprises one or more sgRNAs targeting exon 3 of cTNFRSF1B. In some embodiments, the CRISPR gene-editing system for the treatment of psoriatic arthritis comprises one or more sgRNAs targeting exon 4 of cTNFRSF1B. In some embodiments, the CRISPR gene-editing system for the treatment of psoriatic arthritis comprises one or more sgRNAs targeting exon 5 of cTNFRSF1B. In some embodiments, the CRISPR gene-editing system for the treatment of psoriatic arthritis comprises one or more sgRNAs targeting exon 6 of cTNFRSF1B. In some embodiments, the CRISPR gene-editing system for the treatment of psoriatic arthritis comprises one or more sgRNAs targeting exon 7 of cTNFRSF1B. In some embodiments, the CRISPR gene-editing system for the treatment of psoriatic arthritis comprises one or more sgRNAs targeting exon 8 of cTNFRSF1B. In some embodiments, the CRISPR gene-editing system for the treatment of psoriatic arthritis comprises one or more sgRNAs targeting exon 9 of cTNFRSF1B. In some embodiments, the CRISPR gene-editing system for the treatment of psoriatic arthritis comprises one or more sgRNAs targeting exon 10 of cTNFRSF1B. In some embodiments, the CRISPR gene-editing system for the treatment of psoriatic arthritis comprises one or more sgRNAs targeting exon 11 of cTNFRSF1B.

In some embodiments, the CRISPR gene-editing system for the treatment of psoriatic arthritis comprises one or more sgRNAs targeting exon 1 of eTNFRSF1B. In some embodiments, the CRISPR gene-editing system for the treatment of psoriatic arthritis comprises one or more sgRNAs targeting exon 2 of eTNFRSF1B. In some embodiments, the CRISPR gene-editing system for the treatment of psoriatic arthritis comprises one or more sgRNAs targeting exon 3 of eTNFRSF1B. In some embodiments, the CRISPR gene-editing system for the treatment of psoriatic arthritis comprises one or more sgRNAs targeting exon 4 of eTNFRSF1B. In some embodiments, the CRISPR gene-editing system for the treatment of psoriatic arthritis comprises one or more sgRNAs targeting exon 5 of eTNFRSF1B. In some embodiments, the CRISPR gene-editing system for the treatment of psoriatic arthritis comprises one or more sgRNAs targeting exon 6 of eTNFRSF1B. In some embodiments, the CRISPR gene-editing system for the treatment of psoriatic arthritis comprises one or more sgRNAs targeting exon 7 of eTNFRSF1B. In some embodiments, the CRISPR gene-editing system for the treatment of psoriatic arthritis comprises one or more sgRNAs targeting exon 8 of eTNFRSF1B. In some embodiments, the CRISPR gene-editing system for the treatment of psoriatic arthritis comprises one or more sgRNAs targeting exon 9 of eTNFRSF1B. In some embodiments, the CRISPR gene-editing system for the treatment of psoriatic arthritis comprises one or more sgRNAs targeting exon 10 of eTNFRSF1B. In some embodiments, the CRISPR gene-editing system for the treatment of psoriatic arthritis comprises one or more sgRNAs targeting exon 11 of eTNFRSF1B.

In some embodiments, the CRISPR gene-editing system for the treatment of psoriatic arthritis comprises one or more sgRNAs targeting exon 1 of fTNFRSF1B. In some embodiments, the CRISPR gene-editing system for the treatment of psoriatic arthritis comprises one or more sgRNAs targeting exon 2 of fTNFRSF1B. In some embodiments, the CRISPR gene-editing system for the treatment of psoriatic arthritis comprises one or more sgRNAs targeting exon 3 of fTNFRSF1B. In some embodiments, the CRISPR gene-editing system for the treatment of psoriatic arthritis comprises one or more sgRNAs targeting exon 4 of fTNFRSF1B. In some embodiments, the CRISPR gene-editing system for the treatment of psoriatic arthritis comprises one or more sgRNAs targeting exon 5 of fTNFRSF1B. In some embodiments, the CRISPR gene-editing system for the treatment of psoriatic arthritis comprises one or more sgRNAs targeting exon 6 of fTNFRSF1B. In some embodiments, the CRISPR gene-editing system for the treatment of psoriatic arthritis comprises one or more sgRNAs targeting exon 7 of fTNFRSF1B. In some embodiments, the CRISPR gene-editing system for the treatment of psoriatic arthritis comprises one or more sgRNAs targeting exon 8 of fTNFRSF1B. In some embodiments, the CRISPR gene-editing system for the treatment of psoriatic arthritis comprises one or more sgRNAs targeting exon 9 of fTNFRSF1B. In some embodiments, the CRISPR gene-editing system for the treatment of psoriatic arthritis comprises one or more sgRNAs targeting exon 10 of fTNFRSF1B.

In some embodiments, the CRISPR gene-editing system for the treatment of psoriatic arthritis comprises one or more sgRNAs targeting TNFRSF3. In some embodiments, the CRISPR gene-editing system for the treatment of psoriatic arthritis comprises one or more sgRNAs targeting hTNFRSF3. In some embodiments, the CRISPR gene-editing system for the treatment of psoriatic arthritis comprises one or more sgRNAs targeting cTNFRSF3. In some embodiments, the CRISPR gene-editing system for the treatment of psoriatic arthritis comprises one or more sgRNAs targeting eTNFRSF3. In some embodiments, the CRISPR gene-editing system for the treatment of psoriatic arthritis comprises one or more sgRNAs targeting fTNFRSF3.

In some embodiments, the CRISPR gene-editing system for the treatment of psoriatic arthritis comprises one or more sgRNAs targeting exon 1 of hTNFRSF3. In some embodiments, the CRISPR gene-editing system for the treatment of psoriatic arthritis comprises one or more sgRNAs targeting exon 2 of hTNFRSF3. In some embodiments, the CRISPR gene-editing system for the treatment of psoriatic arthritis comprises one or more sgRNAs targeting exon 3 of hTNFRSF3. In some embodiments, the CRISPR gene-editing system for the treatment of psoriatic arthritis comprises one or more sgRNAs targeting exon 4 of hTNFRSF3. In some embodiments, the CRISPR gene-editing system for the treatment of psoriatic arthritis comprises one or more sgRNAs targeting exon 5 of hTNFRSF3. In some embodiments, the CRISPR gene-editing system for the treatment of psoriatic arthritis comprises one or more sgRNAs targeting exon 6 of hTNFRSF3. In some embodiments, the CRISPR gene-editing system for the treatment of psoriatic arthritis comprises one or more sgRNAs targeting exon 7 of hTNFRSF3. In some embodiments, the CRISPR gene-editing system for the treatment of psoriatic arthritis comprises one or more sgRNAs targeting exon 8 of hTNFRSF3. In some embodiments, the CRISPR gene-editing system for the treatment of psoriatic arthritis comprises one or more sgRNAs targeting exon 9 of hTNFRSF3. In some embodiments, the CRISPR gene-editing system for the treatment of psoriatic arthritis comprises one or more sgRNAs targeting exon 10 of hTNFRSF3. In some embodiments, the CRISPR gene-editing system for the treatment of psoriatic arthritis comprises one or more sgRNAs targeting exon 11 of hTNFRSF3. In some embodiments, the CRISPR gene-editing system for the treatment of psoriatic arthritis comprises one or more sgRNAs targeting exon 12 of hTNFRSF3.

In some embodiments, the CRISPR gene-editing system for the treatment of psoriatic arthritis comprises one or more sgRNAs targeting exon 1 of cTNFRSF3. In some embodiments, the CRISPR gene-editing system for the treatment of psoriatic arthritis comprises one or more sgRNAs targeting exon 2 of cTNFRSF3. In some embodiments, the CRISPR gene-editing system for the treatment of psoriatic arthritis comprises one or more sgRNAs targeting exon 3 of cTNFRSF3. In some embodiments, the CRISPR gene-editing system for the treatment of psoriatic arthritis comprises one or more sgRNAs targeting exon 4 of cTNFRSF3. In some embodiments, the CRISPR gene-editing system for the treatment of psoriatic arthritis comprises one or more sgRNAs targeting exon 5 of cTNFRSF3. In some embodiments, the CRISPR gene-editing system for the treatment of psoriatic arthritis comprises one or more sgRNAs targeting exon 6 of cTNFRSF3. In some embodiments, the CRISPR gene-editing system for the treatment of psoriatic arthritis comprises one or more sgRNAs targeting exon 7 of cTNFRSF3. In some embodiments, the CRISPR gene-editing system for the treatment of psoriatic arthritis comprises one or more sgRNAs targeting exon 8 of cTNFRSF3. In some embodiments, the CRISPR gene-editing system for the treatment of psoriatic arthritis comprises one or more sgRNAs targeting exon 9 of cTNFRSF3. In some embodiments, the CRISPR gene-editing system for the treatment of psoriatic arthritis comprises one or more sgRNAs targeting exon 10 of cTNFRSF3.

In some embodiments, the CRISPR gene-editing system for the treatment of psoriatic arthritis comprises one or more sgRNAs targeting exon 1 of eTNFRSF3. In some embodiments, the CRISPR gene-editing system for the treatment of psoriatic arthritis comprises one or more sgRNAs targeting exon 2 of eTNFRSF3. In some embodiments, the CRISPR gene-editing system for the treatment of psoriatic arthritis comprises one or more sgRNAs targeting exon 3 of eTNFRSF3. In some embodiments, the CRISPR gene-editing system for the treatment of psoriatic arthritis comprises one or more sgRNAs targeting exon 4 of eTNFRSF3. In some embodiments, the CRISPR gene-editing system for the treatment of psoriatic arthritis comprises one or more sgRNAs targeting exon 5 of eTNFRSF3. In some embodiments, the CRISPR gene-editing system for the treatment of psoriatic arthritis comprises one or more sgRNAs targeting exon 6 of eTNFRSF3. In some embodiments, the CRISPR gene-editing system for the treatment of psoriatic arthritis comprises one or more sgRNAs targeting exon 7 of eTNFRSF3. In some embodiments, the CRISPR gene-editing system for the treatment of psoriatic arthritis comprises one or more sgRNAs targeting exon 8 of eTNFRSF3. In some embodiments, the CRISPR gene-editing system for the treatment of psoriatic arthritis comprises one or more sgRNAs targeting exon 9 of eTNFRSF3. In some embodiments, the CRISPR gene-editing system for the treatment of psoriatic arthritis comprises one or more sgRNAs targeting exon 10 of eTNFRSF3.

In some embodiments, the CRISPR gene-editing system for the treatment of psoriatic arthritis comprises one or more sgRNAs targeting exon 1 of fTNFRSF3. In some embodiments, the CRISPR gene-editing system for the treatment of psoriatic arthritis comprises one or more sgRNAs targeting exon 2 of fTNFRSF3. In some embodiments, the CRISPR gene-editing system for the treatment of psoriatic arthritis comprises one or more sgRNAs targeting exon 3 of fTNFRSF3. In some embodiments, the CRISPR gene-editing system for the treatment of psoriatic arthritis comprises one or more sgRNAs targeting exon 4 of fTNFRSF3. In some embodiments, the CRISPR gene-editing system for the treatment of psoriatic arthritis comprises one or more sgRNAs targeting exon 5 of fTNFRSF3. In some embodiments, the CRISPR gene-editing system for the treatment of psoriatic arthritis comprises one or more sgRNAs targeting exon 6 of fTNFRSF3. In some embodiments, the CRISPR gene-editing system for the treatment of psoriatic arthritis comprises one or more sgRNAs targeting exon 7 of fTNFRSF3. In some embodiments, the CRISPR gene-editing system for the treatment of psoriatic arthritis comprises one or more sgRNAs targeting exon 8 of fTNFRSF3. In some embodiments, the CRISPR gene-editing system for the treatment of psoriatic arthritis comprises one or more sgRNAs targeting exon 9 of fTNFRSF3. In some embodiments, the CRISPR gene-editing system for the treatment of psoriatic arthritis comprises one or more sgRNAs targeting exon 10 of fTNFRSF3.

In some embodiments, the CRISPR gene-editing system for the treatment of psoriatic arthritis comprises one or more sgRNAs targeting TNFRSF4. In some embodiments, the CRISPR gene-editing system for the treatment of psoriatic arthritis comprises one or more sgRNAs targeting hTNFRSF4. In some embodiments, the CRISPR gene-editing system for the treatment of psoriatic arthritis comprises one or more sgRNAs targeting cTNFRSF4. In some embodiments, the CRISPR gene-editing system for the treatment of psoriatic arthritis comprises one or more sgRNAs targeting eTNFRSF4. In some embodiments, the CRISPR gene-editing system for the treatment of psoriatic arthritis comprises one or more sgRNAs targeting fTNFRSF4.

In some embodiments, the CRISPR gene-editing system for the treatment of psoriatic arthritis comprises one or more sgRNAs targeting exon 1 of hTNFRSF4. In some embodiments, the CRISPR gene-editing system for the treatment of psoriatic arthritis comprises one or more sgRNAs targeting exon 2 of hTNFRSF4. In some embodiments, the CRISPR gene-editing system for the treatment of psoriatic arthritis comprises one or more sgRNAs targeting exon 3 of hTNFRSF4. In some embodiments, the CRISPR gene-editing system for the treatment of psoriatic arthritis comprises one or more sgRNAs targeting exon 4 of hTNFRSF4. In some embodiments, the CRISPR gene-editing system for the treatment of psoriatic arthritis comprises one or more sgRNAs targeting exon 5 of hTNFRSF4. In some embodiments, the CRISPR gene-editing system for the treatment of psoriatic arthritis comprises one or more sgRNAs targeting exon 6 of hTNFRSF4.

In some embodiments, the CRISPR gene-editing system for the treatment of psoriatic arthritis comprises one or more sgRNAs targeting exon 1 of cTNFRSF4. In some embodiments, the CRISPR gene-editing system for the treatment of psoriatic arthritis comprises one or more sgRNAs targeting exon 2 of cTNFRSF4. In some embodiments, the CRISPR gene-editing system for the treatment of psoriatic arthritis comprises one or more sgRNAs targeting exon 3 of cTNFRSF4. In some embodiments, the CRISPR gene-editing system for the treatment of psoriatic arthritis comprises one or more sgRNAs targeting exon 4 of cTNFRSF4. In some embodiments, the CRISPR gene-editing system for the treatment of psoriatic arthritis comprises one or more sgRNAs targeting exon 5 of cTNFRSF4. In some embodiments, the CRISPR gene-editing system for the treatment of psoriatic arthritis comprises one or more sgRNAs targeting exon 6 of cTNFRSF4. In some embodiments, the CRISPR gene-editing system for the treatment of psoriatic arthritis comprises one or more sgRNAs targeting exon 7 of cTNFRSF4.

In some embodiments, the CRISPR gene-editing system for the treatment of psoriatic arthritis comprises one or more sgRNAs targeting exon 1 of eTNFRSF4. In some embodiments, the CRISPR gene-editing system for the treatment of psoriatic arthritis comprises one or more sgRNAs targeting exon 2 of eTNFRSF4. In some embodiments, the CRISPR gene-editing system for the treatment of psoriatic arthritis comprises one or more sgRNAs targeting exon 3 of eTNFRSF4. In some embodiments, the CRISPR gene-editing system for the treatment of psoriatic arthritis comprises one or more sgRNAs targeting exon 4 of eTNFRSF4. In some embodiments, the CRISPR gene-editing system for the treatment of psoriatic arthritis comprises one or more sgRNAs targeting exon 5 of eTNFRSF4. In some embodiments, the CRISPR gene-editing system for the treatment of psoriatic arthritis comprises one or more sgRNAs targeting exon 6 of eTNFRSF4. In some embodiments, the CRISPR gene-editing system for the treatment of psoriatic arthritis comprises one or more sgRNAs targeting exon 7 of eTNFRSF4.

In some embodiments, the CRISPR gene-editing system for the treatment of psoriatic arthritis comprises one or more sgRNAs targeting exon 1 of fTNFRSF4. In some embodiments, the CRISPR gene-editing system for the treatment of psoriatic arthritis comprises one or more sgRNAs targeting exon 2 of fTNFRSF4. In some embodiments, the CRISPR gene-editing system for the treatment of psoriatic arthritis comprises one or more sgRNAs targeting exon 3 of fTNFRSF4. In some embodiments, the CRISPR gene-editing system for the treatment of psoriatic arthritis comprises one or more sgRNAs targeting exon 4 of fTNFRSF4. In some embodiments, the CRISPR gene-editing system for the treatment of psoriatic arthritis comprises one or more sgRNAs targeting exon 5 of fTNFRSF4. In some embodiments, the CRISPR gene-editing system for the treatment of psoriatic arthritis comprises one or more sgRNAs targeting exon 6 of fTNFRSF4. In some embodiments, the CRISPR gene-editing system for the treatment of psoriatic arthritis comprises one or more sgRNAs targeting exon 7 of fTNFRSF4.

In some embodiments, the CRISPR gene-editing system for the treatment of psoriatic arthritis comprises one or more sgRNAs targeting TNFRSF11A. In some embodiments, the CRISPR gene-editing system for the treatment of psoriatic arthritis comprises one or more sgRNAs targeting hTNFRSF11A. In some embodiments, the CRISPR gene-editing system for the treatment of psoriatic arthritis comprises one or more sgRNAs targeting cTNFRSF11A. In some embodiments, the CRISPR gene-editing system for the treatment of psoriatic arthritis comprises one or more sgRNAs targeting eTNFRSF11A. In some embodiments, the CRISPR gene-editing system for the treatment of psoriatic arthritis comprises one or more sgRNAs targeting fTNFRSF11A.

In some embodiments, the CRISPR gene-editing system for the treatment of psoriatic arthritis comprises one or more sgRNAs targeting exon 1 of hTNFRSF11A. In some embodiments, the CRISPR gene-editing system for the treatment of psoriatic arthritis comprises one or more sgRNAs targeting exon 2 of hTNFRSF11A. In some embodiments, the CRISPR gene-editing system for the treatment of psoriatic arthritis comprises one or more sgRNAs targeting exon 3 of hTNFRSF11A. In some embodiments, the CRISPR gene-editing system for the treatment of psoriatic arthritis comprises one or more sgRNAs targeting exon 4 of hTNFRSF11A. In some embodiments, the CRISPR gene-editing system for the treatment of psoriatic arthritis comprises one or more sgRNAs targeting exon 5 of hTNFRSF11A. In some embodiments, the CRISPR gene-editing system for the treatment of psoriatic arthritis comprises one or more sgRNAs targeting exon 6 of hTNFRSF11A. In some embodiments, the CRISPR gene-editing system for the treatment of psoriatic arthritis comprises one or more sgRNAs targeting exon 7 of hTNFRSF11A. In some embodiments, the CRISPR gene-editing system for the treatment of psoriatic arthritis comprises one or more sgRNAs targeting exon 8 of hTNFRSF11A. In some embodiments, the CRISPR gene-editing system for the treatment of psoriatic arthritis comprises one or more sgRNAs targeting exon 9 of hTNFRSF11A. In some embodiments, the CRISPR gene-editing system for the treatment of psoriatic arthritis comprises one or more sgRNAs targeting exon 10 of hTNFRSF11A. In some embodiments, the CRISPR gene-editing system for the treatment of psoriatic arthritis comprises one or more sgRNAs targeting exon 11 of hTNFRSF11A. In some embodiments, the CRISPR gene-editing system for the treatment of psoriatic arthritis comprises one or more sgRNAs targeting exon 12 of hTNFRSF11A.

In some embodiments, the CRISPR gene-editing system for the treatment of psoriatic arthritis comprises one or more sgRNAs targeting exon 1 of cTNFRSF11A. In some embodiments, the CRISPR gene-editing system for the treatment of psoriatic arthritis comprises one or more sgRNAs targeting exon 2 of cTNFRSF11A. In some embodiments, the CRISPR gene-editing system for the treatment of psoriatic arthritis comprises one or more sgRNAs targeting exon 3 of cTNFRSF11A. In some embodiments, the CRISPR gene-editing system for the treatment of psoriatic arthritis comprises one or more sgRNAs targeting exon 4 of cTNFRSF11A. In some embodiments, the CRISPR gene-editing system for the treatment of psoriatic arthritis comprises one or more sgRNAs targeting exon 5 of cTNFRSF11A. In some embodiments, the CRISPR gene-editing system for the treatment of psoriatic arthritis comprises one or more sgRNAs targeting exon 6 of cTNFRSF11A. In some embodiments, the CRISPR gene-editing system for the treatment of psoriatic arthritis comprises one or more sgRNAs targeting exon 7 of cTNFRSF11A. In some embodiments, the CRISPR gene-editing system for the treatment of psoriatic arthritis comprises one or more sgRNAs targeting exon 8 of cTNFRSF11A. In some embodiments, the CRISPR gene-editing system for the treatment of psoriatic arthritis comprises one or more sgRNAs targeting exon 9 of cTNFRSF11A. In some embodiments, the CRISPR gene-editing system for the treatment of psoriatic arthritis comprises one or more sgRNAs targeting exon 10 of cTNFRSF11A. In some embodiments, the CRISPR gene-editing system for the treatment of psoriatic arthritis comprises one or more sgRNAs targeting exon 11 of cTNFRSF11A.

In some embodiments, the CRISPR gene-editing system for the treatment of psoriatic arthritis comprises one or more sgRNAs targeting exon 1 of eTNFRSF11A. In some embodiments, the CRISPR gene-editing system for the treatment of psoriatic arthritis comprises one or more sgRNAs targeting exon 2 of eTNFRSF11A. In some embodiments, the CRISPR gene-editing system for the treatment of psoriatic arthritis comprises one or more sgRNAs targeting exon 3 of eTNFRSF11A. In some embodiments, the CRISPR gene-editing system for the treatment of psoriatic arthritis comprises one or more sgRNAs targeting exon 4 of eTNFRSF11A. In some embodiments, the CRISPR gene-editing system for the treatment of psoriatic arthritis comprises one or more sgRNAs targeting exon 5 of eTNFRSF11A. In some embodiments, the CRISPR gene-editing system for the treatment of psoriatic arthritis comprises one or more sgRNAs targeting exon 6 of eTNFRSF11A. In some embodiments, the CRISPR gene-editing system for the treatment of psoriatic arthritis comprises one or more sgRNAs targeting exon 7 of eTNFRSF11A. In some embodiments, the CRISPR gene-editing system for the treatment of psoriatic arthritis comprises one or more sgRNAs targeting exon 8 of eTNFRSF11A. In some embodiments, the CRISPR gene-editing system for the treatment of psoriatic arthritis comprises one or more sgRNAs targeting exon 9 of eTNFRSF11A. In some embodiments, the CRISPR gene-editing system for the treatment of psoriatic arthritis comprises one or more sgRNAs targeting exon 10 of eTNFRSF11A.

In some embodiments, the CRISPR gene-editing system for the treatment of psoriatic arthritis comprises one or more sgRNAs targeting exon 1 of fTNFRSF11A. In some embodiments, the CRISPR gene-editing system for the treatment of psoriatic arthritis comprises one or more sgRNAs targeting exon 2 of fTNFRSF11A. In some embodiments, the CRISPR gene-editing system for the treatment of psoriatic arthritis comprises one or more sgRNAs targeting exon 3 of fTNFRSF11A. In some embodiments, the CRISPR gene-editing system for the treatment of psoriatic arthritis comprises one or more sgRNAs targeting exon 4 of fTNFRSF11A. In some embodiments, the CRISPR gene-editing system for the treatment of psoriatic arthritis comprises one or more sgRNAs targeting exon 5 of fTNFRSF11A. In some embodiments, the CRISPR gene-editing system for the treatment of psoriatic arthritis comprises one or more sgRNAs targeting exon 6 of fTNFRSF11A. In some embodiments, the CRISPR gene-editing system for the treatment of psoriatic arthritis comprises one or more sgRNAs targeting exon 7 of fTNFRSF11A. In some embodiments, the CRISPR gene-editing system for the treatment of psoriatic arthritis comprises one or more sgRNAs targeting exon 8 of fTNFRSF11A. In some embodiments, the CRISPR gene-editing system for the treatment of psoriatic arthritis comprises one or more sgRNAs targeting exon 9 of fTNFRSF11A. In some embodiments, the CRISPR gene-editing system for the treatment of psoriatic arthritis comprises one or more sgRNAs targeting exon 10 of fTNFRSF11A. In some embodiments, the CRISPR gene-editing system for the treatment of psoriatic arthritis comprises one or more sgRNAs targeting exon 11 of fTNFRSF11A.

### D. Gout

In one aspect, the present disclosure encompasses treatments for gout and other crystallopathies affecting the joint, e.g., octacalcium phosphate and calcium pyrophosphate dihydrate in horses. In some embodiments, the gout treatment comprises a therapeutically effective amount of a Clustered Regularly Interspaced Short Palindromic Repeats (CRISPR) gene-editing system, the system comprising: (i) a CRISPR Associated (Cas) protein; and (ii) at least one guide RNA targeting IL1R1. In some embodiments, the gout treatment comprises a CRISPR gene-editing system targeting hIL1R1. In some embodiments, the gout treatment comprises a CRISPR gene-editing system targeting cIL1R1. In some embodiments, the gout treatment comprises a CRISP gene-editing system R targeting eIL1R1. In some embodiments, the gout treatment comprises a CRISPR gene-editing system targeting fIL1R1.

In some embodiments, the CRISPR gene-editing system for the treatment of gout comprises one or more sgRNAs targeting exon 1 of hIL1R1. In some embodiments, the CRISPR gene-editing system for the treatment of gout comprises one or more sgRNAs targeting exon 2 of hIL1R1. In some embodiments, the CRISPR gene-editing system for the treatment of gout comprises one or more sgRNAs targeting exon 3 of hIL1R1. In some embodiments, the CRISPR gene-editing system for the treatment of gout comprises one or more sgRNAs targeting exon 4 of hIL1R1. In some embodiments, the CRISPR gene-editing system for the treatment of gout comprises one or more sgRNAs targeting exon 5 of hIL1R1. In some embodiments, the CRISPR gene-editing system for the treatment of gout comprises one or more sgRNAs targeting exon 6 of hIL1R1. In some embodiments, the CRISPR gene-editing system for the treatment of gout comprises one or more sgRNAs targeting exon 7 of hIL1R1. In some embodiments, the CRISPR gene-editing system for the treatment of gout comprises one or more sgRNAs targeting exon 8 of hIL1R1. In some embodiments, the CRISPR gene-editing system for the treatment of gout comprises one or more sgRNAs targeting exon 9 of hIL1R1. In some embodiments, the CRISPR gene-editing system for the treatment of gout comprises one or more sgRNAs targeting exon 10 of hIL1R1. In some embodiments, the CRISPR gene-editing system for the treatment of gout comprises one or more sgRNAs targeting exon 11 of hIL1R1. In some embodiments, the CRISPR gene-editing system for the treatment of gout comprises one or more sgRNAs targeting exon 12 of hIL1R1. In some embodiments, the CRISPR gene-editing system for the treatment of gout comprises one or more sgRNAs targeting exon 13 of hIL1R1. In some embodiments, the CRISPR gene-editing system for the treatment of gout comprises one or more sgRNAs targeting exon 14 of hIL1R1. In some embodiments, the CRISPR gene-editing system for the treatment of gout comprises one or more sgRNAs targeting exon 15 of hIL1R1. In some embodiments, the CRISPR gene-editing system for the treatment of gout comprises one or more sgRNAs targeting exon 16 of hIL1R1. In some embodiments, the CRISPR gene-editing system for the treatment of gout comprises one or more sgRNAs targeting exon 17 of hIL1R1. In some embodiments, the CRISPR gene-editing system for the treatment of gout comprises one or more sgRNAs targeting exon 18 of hIL1R1. In some embodiments, the CRISPR gene-editing system for the treatment of gout comprises one or more sgRNAs targeting exon 19 of hIL1R1. In some embodiments, the CRISPR gene-editing system for the treatment of gout comprises one or more sgRNAs targeting exon 20 of hIL1R1. In some embodiments, the CRISPR gene-editing system for the treatment of gout comprises one or more sgRNAs targeting exon 21 of hIL1R1.

In some embodiments, the CRISPR gene-editing system for the treatment of gout comprises one or more sgRNAs targeting exon 1 of cIL1R1. In some embodiments, the CRISPR gene-editing system for the treatment of gout comprises one or more sgRNAs targeting exon 2 of cIL1R1. In some embodiments, the CRISPR gene-editing system for the treatment of gout comprises one or more sgRNAs targeting exon 3 of cIL1R1. In some embodiments, the CRISPR gene-editing system for the treatment of gout comprises one or more sgRNAs targeting exon 4 of cIL1R1. In some embodiments, the CRISPR gene-editing system for the treatment of gout comprises one or more sgRNAs targeting exon 5 of cIL1R1. In some embodiments, the CRISPR gene-editing system for the treatment of gout comprises one or more sgRNAs targeting exon 6 of cIL1R1. In some embodiments, the CRISPR gene-editing system for the treatment of gout comprises one or more sgRNAs targeting exon 7 of cIL1R1. In some embodiments, the CRISPR gene-editing system for the treatment of gout comprises one or more sgRNAs targeting exon 8 of cIL1R1. In some embodiments, the CRISPR gene-editing system for the treatment of gout comprises one or more sgRNAs targeting exon 9 of cIL1R1. In some embodiments, the CRISPR gene-editing system for the treatment of gout comprises one or more sgRNAs targeting exon 10 of cIL1R1. In some embodiments, the CRISPR gene-editing system for the treatment of gout comprises one or more sgRNAs targeting exon 11 of cIL1R1. In some embodiments, the CRISPR gene-editing system for the treatment of gout comprises one or more sgRNAs targeting exon 12 of cIL1R1. In some embodiments, the CRISPR gene-editing system for the treatment of gout comprises one or more sgRNAs targeting exon 13 of cIL1R1. In some embodiments, the CRISPR gene-editing system for the treatment of gout comprises one or more sgRNAs targeting exon 14 of cIL1R1. In some embodiments, the CRISPR gene-editing system for the treatment of gout comprises one or more sgRNAs targeting exon 15 of cIL1R1. In some embodiments, the CRISPR gene-editing system for the treatment of gout comprises one or more sgRNAs targeting exon 16 of cIL1R1.

In some embodiments, the CRISPR gene-editing system for the treatment of gout comprises one or more sgRNAs targeting exon 1 of eIL1R1. In some embodiments, the CRISPR gene-editing system for the treatment of gout comprises one or more sgRNAs targeting exon 2 of eIL1R1. In some embodiments, the CRISPR gene-editing system for the treatment of gout comprises one or more sgRNAs targeting exon 3 of eIL1R1. In some embodiments, the CRISPR gene-editing system for the treatment of gout comprises one or more sgRNAs targeting exon 4 of eIL1R1. In some embodiments, the CRISPR gene-editing system for the treatment of gout comprises one or more sgRNAs targeting exon 5 of eIL1R1. In some embodiments, the CRISPR gene-editing system for the treatment of gout comprises one or more sgRNAs targeting exon 6 of eIL1R1. In some embodiments, the CRISPR gene-editing system for the treatment of gout comprises one or more sgRNAs targeting exon 7 of eIL1R1. In some embodiments, the CRISPR gene-editing system for the treatment of gout comprises one or more sgRNAs targeting exon 8 of eIL1R1. In some embodiments, the CRISPR gene-editing system for the treatment of gout comprises one or more sgRNAs targeting exon 9 of eIL1R1. In some embodiments, the CRISPR gene-editing system for the treatment of gout comprises one or more sgRNAs targeting exon 10 of eIL1R1. In some embodiments, the CRISPR gene-editing system for the treatment of gout comprises one or more sgRNAs targeting exon 11 of eIL1R1. In some embodiments, the CRISPR gene-editing system for the treatment of gout comprises one or more sgRNAs targeting exon 12 of eIL1R1. In some embodiments, the CRISPR gene-editing system for the treatment of gout comprises one or more sgRNAs targeting exon 13 of eIL1R1. In some embodiments, the CRISPR gene-editing system for the treatment of gout comprises one or more sgRNAs targeting exon 14 of eIL1R1. In some embodiments, the CRISPR gene-editing system for the treatment of gout comprises one or more sgRNAs targeting exon 15 of eIL1R1.

In some embodiments, the CRISPR gene-editing system for the treatment of gout comprises one or more sgRNAs targeting exon 1 of fIL1R1. In some embodiments, the CRISPR gene-editing system for the treatment of gout comprises one or more sgRNAs targeting exon 2 of fIL1R1. In some embodiments, the CRISPR gene-editing system for the treatment of gout comprises one or more sgRNAs targeting exon 3 of fIL1R1. In some embodiments, the CRISPR gene-editing system for the treatment of gout comprises one or more sgRNAs targeting exon 4 of fIL1R1. In some embodiments, the CRISPR gene-editing system for the treatment of gout comprises one or more sgRNAs targeting exon 5 of fIL1R1. In some embodiments, the CRISPR gene-editing system for the treatment of gout comprises one or more sgRNAs targeting exon 6 of fIL1R1. In some embodiments, the CRISPR gene-editing system for the treatment of gout comprises one or more sgRNAs targeting exon 7 of fIL1R1. In some embodiments, the CRISPR gene-editing system for the treatment of gout comprises one or more sgRNAs targeting exon 8 of fIL1R1. In some embodiments, the CRISPR gene-editing system for the treatment of gout comprises one or more sgRNAs targeting exon 9 of fIL1R1. In some embodiments, the CRISPR gene-editing system for the treatment of gout comprises one or more sgRNAs targeting exon 10 of fIL1R1. In some embodiments, the CRISPR gene-editing system for the treatment of gout comprises one or more sgRNAs targeting exon 11 of fIL1R1. In some embodiments, the CRISPR gene-editing system for the treatment of gout comprises one or more sgRNAs targeting exon 12 of fIL1R1. In some embodiments, the CRISPR gene-editing system for the treatment of gout comprises one or more sgRNAs targeting exon 13 of fIL1R1. In some embodiments, the CRISPR gene-editing system for the treatment of gout comprises one or more sgRNAs targeting exon 14 of fIL1R1. In some embodiments, the CRISPR gene-editing system for the treatment of gout comprises one or more sgRNAs targeting exon 15 of fIL1R1. In some embodiments, the CRISPR gene-editing system for the treatment of gout comprises one or more sgRNAs targeting exon 16 of fIL1R1. In some embodiments, the CRISPR gene-editing system for the treatment of gout comprises one or more sgRNAs targeting exon 17 of fIL1R1. In some embodiments, the CRISPR gene-editing system for the treatment of gout comprises one or more sgRNAs targeting exon 18 of fIL1R1.

In some embodiments, the CRISPR gene-editing system for the treatment of gout comprises one or more sgRNAs targeting IL1RAP. In some embodiments, the CRISPR gene-editing system for the treatment of gout comprises one or more sgRNAs targeting hIL1RAP. In some embodiments, the CRISPR gene-editing system for the treatment of gout comprises one or more sgRNAs targeting cIL1RAP. In some embodiments, the CRISPR gene-editing system for the treatment of gout comprises one or more sgRNAs targeting eIL1RAP. In some embodiments, the CRISPR gene-editing system for the treatment of gout comprises one or more sgRNAs targeting fIL1RAP.

In some embodiments, the CRISPR gene-editing system for the treatment of gout comprises one or more sgRNAs targeting exon 1 of hIL1RAP. In some embodiments, the CRISPR gene-editing system for the treatment of gout comprises one or more sgRNAs targeting exon 2 of hIL1RAP. In some embodiments, the CRISPR gene-editing system for the treatment of gout comprises one or more sgRNAs targeting exon 3 of hIL1RAP. In some embodiments, the CRISPR gene-editing system for the treatment of gout comprises one or more sgRNAs targeting exon 4 of hIL1RAP. In some embodiments, the CRISPR gene-editing system for the treatment of gout comprises one or more sgRNAs targeting exon 5 of hIL1RAP. In some embodiments, the CRISPR gene-editing system for the treatment of gout comprises one or more sgRNAs targeting exon 6 of hIL1RAP. In some embodiments, the CRISPR gene-editing system for the treatment of gout comprises one or more sgRNAs targeting exon 7 of hIL1RAP. In some embodiments, the CRISPR gene-editing system for the treatment of gout comprises one or more sgRNAs targeting exon 8 of hIL1RAP. In some embodiments, the CRISPR gene-editing system for the treatment of gout comprises one or more sgRNAs targeting exon 9 of hIL1RAP. In some embodiments, the CRISPR gene-editing system for the treatment of gout comprises one or more sgRNAs targeting exon 10 of hIL1RAP. In some embodiments, the CRISPR gene-editing system for the treatment of gout comprises one or more sgRNAs targeting exon 11 of hIL1RAP. In some embodiments, the CRISPR gene-editing system for the treatment of gout comprises one or more sgRNAs targeting exon 12 of hIL1RAP. In some embodiments, the CRISPR gene-editing system for the treatment of gout comprises one or more sgRNAs targeting exon 13 of hIL1RAP. In some embodiments, the CRISPR gene-editing system for the treatment of gout comprises one or more sgRNAs targeting exon 14 of hIL1RAP. In some embodiments, the CRISPR gene-editing system for the treatment of gout comprises one or more sgRNAs targeting exon 15 of hIL1RAP. In some embodiments, the CRISPR gene-editing system for the treatment of gout comprises one or more sgRNAs targeting exon 16 of hIL1RAP. In some embodiments, the CRISPR gene-editing system for the treatment of gout comprises one or more sgRNAs targeting exon 17 of hIL1RAP.

In some embodiments, the CRISPR gene-editing system for the treatment of gout comprises one or more sgRNAs targeting exon 1 of cIL1RAP. In some embodiments, the CRISPR gene-editing system for the treatment of gout comprises one or more sgRNAs targeting exon 2 of cIL1RAP. In some embodiments, the CRISPR gene-editing system for the treatment of gout comprises one or more sgRNAs targeting exon 3 of cIL1RAP. In some embodiments, the CRISPR gene-editing system for the treatment of gout comprises one or more sgRNAs targeting exon 4 of cIL1RAP. In some embodiments, the CRISPR gene-editing system for the treatment of gout comprises one or more sgRNAs targeting exon 5 of cIL1RAP. In some embodiments, the CRISPR gene-editing system for the treatment of gout comprises one or more sgRNAs targeting exon 6 of cIL1RAP. In some embodiments, the CRISPR gene-editing system for the treatment of gout comprises one or more sgRNAs targeting exon 7 of cIL1RAP. In some embodiments, the CRISPR gene-editing system for the treatment of gout comprises one or more sgRNAs targeting exon 8 of cIL1RAP. In some embodiments, the CRISPR gene-editing system for the treatment of gout comprises one or more sgRNAs targeting exon 9 of cIL1RAP. In some embodiments, the CRISPR gene-editing system for the treatment of gout comprises one or more sgRNAs targeting exon 10 of cIL1RAP. In some embodiments, the CRISPR gene-editing system for the treatment of gout comprises one or more sgRNAs targeting exon 11 of cIL1RAP. In some embodiments, the CRISPR gene-editing system for the treatment of gout comprises one or more sgRNAs targeting exon 12 of cIL1RAP. In some embodiments, the CRISPR gene-editing system for the treatment of gout comprises one or more sgRNAs targeting exon 13 of cIL1RAP. In some embodiments, the CRISPR gene-editing system for the treatment of gout comprises one or more sgRNAs targeting exon 14 of cIL1RAP. In some embodiments, the CRISPR gene-editing system for the treatment of gout comprises one or more sgRNAs targeting exon 15 of cIL1RAP. In some embodiments, the CRISPR gene-editing system for the treatment of gout comprises one or more sgRNAs targeting exon 16 of cIL1RAP.

In some embodiments, the CRISPR gene-editing system for the treatment of gout comprises one or more sgRNAs targeting exon 1 of eIL1RAP. In some embodiments, the CRISPR gene-editing system for the treatment of gout comprises one or more sgRNAs targeting exon 2 of eIL1RAP. In some embodiments, the CRISPR gene-editing system for the treatment of gout comprises one or more sgRNAs targeting exon 3 of eIL1RAP. In some embodiments, the CRISPR gene-editing system for the treatment of gout comprises one or more sgRNAs targeting exon 4 of eIL1RAP. In some embodiments, the CRISPR gene-editing system for the treatment of gout comprises one or more sgRNAs targeting exon 5 of eIL1RAP. In some embodiments, the CRISPR gene-editing system for the treatment of gout comprises one or more sgRNAs targeting exon 6 of eIL1RAP. In some embodiments, the CRISPR gene-editing system for the treatment of gout comprises one or more sgRNAs targeting exon 7 of eIL1RAP. In some embodiments, the CRISPR gene-editing system for the treatment of gout comprises one or more sgRNAs targeting exon 8 of eIL1RAP. In some embodiments, the CRISPR gene-editing system for the treatment of gout comprises one or more sgRNAs targeting exon 9 of eIL1RAP. In some embodiments, the CRISPR gene-editing system for the treatment of gout comprises one or more sgRNAs targeting exon 10 of eIL1RAP. In some embodiments, the CRISPR gene-editing system for the treatment of gout comprises one or more sgRNAs targeting exon 11 of eIL1RAP. In some embodiments, the CRISPR gene-editing system for the treatment of gout comprises one or more sgRNAs targeting exon 12 of eIL1RAP. In some embodiments, the CRISPR gene-editing system for the treatment of gout comprises one or more sgRNAs targeting exon 13 of eIL1RAP.

In some embodiments, the CRISPR gene-editing system for the treatment of gout comprises one or more sgRNAs targeting exon 1 of fIL1RAP. In some embodiments, the CRISPR gene-editing system for the treatment of gout comprises one or more sgRNAs targeting exon 2 of fIL1RAP. In some embodiments, the CRISPR gene-editing system for the treatment of gout comprises one or more sgRNAs targeting exon 3 of fIL1RAP. In some embodiments, the CRISPR gene-editing system for the treatment of gout comprises one or more sgRNAs targeting exon 4 of fIL1RAP. In some embodiments, the CRISPR gene-editing system for the treatment of gout comprises one or more sgRNAs targeting exon 5 of fIL1RAP. In some embodiments, the CRISPR gene-editing system for the treatment of gout comprises one or more sgRNAs targeting exon 6 of fIL1RAP. In some embodiments, the CRISPR gene-editing system for the treatment of gout comprises one or more sgRNAs targeting exon 7 of fIL1RAP. In some embodiments, the CRISPR gene-editing system for the treatment of gout comprises one or more sgRNAs targeting exon 8 of fIL1RAP. In some embodiments, the CRISPR gene-editing system for the treatment of gout comprises one or more sgRNAs targeting exon 9 of fIL1RAP. In some embodiments, the CRISPR gene-editing system for the treatment of gout comprises one or more sgRNAs targeting exon 10 of fIL1RAP. In some embodiments, the CRISPR gene-editing system for the treatment of gout comprises one or more sgRNAs targeting exon 11 of fIL1RAP. In some embodiments, the CRISPR gene-editing system for the treatment of gout comprises one or more sgRNAs targeting exon 12 of fIL1RAP. In some embodiments, the CRISPR gene-editing system for the treatment of gout comprises one or more sgRNAs targeting exon 13 of fIL1RAP. In some embodiments, the CRISPR gene-editing system for the treatment of gout comprises one or more sgRNAs targeting exon 14 of fIL1RAP.

In some embodiments, the CRISPR gene-editing system for the treatment of gout comprises one or more sgRNAs targeting TGFBR1. In some embodiments, the CRISPR gene-editing system for the treatment of gout comprises one or more sgRNAs targeting hTGFBR1. In some embodiments, the CRISPR gene-editing system for the treatment of gout comprises one or more sgRNAs targeting cTGFBR1. In some embodiments, the CRISPR gene-editing system for the treatment of gout comprises one or more sgRNAs targeting e TGFBR1. In some embodiments, the CRISPR gene-editing system for the treatment of gout comprises one or more sgRNAs targeting fTGFBR1.

In some embodiments, the CRISPR gene-editing system for the treatment of gout comprises one or more sgRNAs targeting exon 1 of hTGFBR1. In some embodiments, the CRISPR gene-editing system for the treatment of gout comprises one or more sgRNAs targeting exon 2 of hTGFBR1. In some embodiments, the CRISPR gene-editing system for the treatment of gout comprises one or more sgRNAs targeting exon 3 of hTGFBR1. In some embodiments, the CRISPR gene-editing system for the treatment of gout comprises one or more sgRNAs targeting exon 4 of hTGFBR1. In some embodiments, the CRISPR gene-editing system for the treatment of gout comprises one or more sgRNAs targeting exon 5 of hTGFBR1. In some embodiments, the CRISPR gene-editing system for the treatment of gout comprises one or more sgRNAs targeting exon 6 of hTGFBR1. In some embodiments, the CRISPR gene-editing system for the treatment of gout comprises one or more sgRNAs targeting exon 7 of hTGFBR1. In some embodiments, the CRISPR gene-editing system for the treatment of gout comprises one or more sgRNAs targeting exon 8 of hTGFBR1. In some embodiments, the CRISPR gene-editing system for the treatment of gout comprises one or more sgRNAs targeting exon 9 of hTGFBR1. In some embodiments, the CRISPR gene-editing system for the treatment of gout comprises one or more sgRNAs targeting exon 10 of hTGFBR1. In some embodiments, the CRISPR gene-editing system for the treatment of gout comprises one or more sgRNAs targeting exon 11 of hTGFBR1.

In some embodiments, the CRISPR gene-editing system for the treatment of gout comprises one or more sgRNAs targeting exon 1 of cTGFBR1. In some embodiments, the CRISPR gene-editing system for the treatment of gout comprises one or more sgRNAs targeting exon 2 of cTGFBR1. In some embodiments, the CRISPR gene-editing system for the treatment of gout comprises one or more sgRNAs targeting exon 3 of cTGFBR1. In some embodiments, the CRISPR gene-editing system for the treatment of gout comprises one or more sgRNAs targeting exon 4 of cTGFBR1. In some embodiments, the CRISPR gene-editing system for the treatment of gout comprises one or more sgRNAs targeting exon 5 of cTGFBR1. In some embodiments, the CRISPR gene-editing system for the treatment of gout comprises one or more sgRNAs targeting exon 6 of cTGFBR1. In some embodiments, the CRISPR gene-editing system for the treatment of gout comprises one or more sgRNAs targeting exon 7 of cTGFBR1. In some embodiments, the CRISPR gene-editing system for the treatment of gout comprises one or more sgRNAs targeting exon 8 of cTGFBR1. In some embodiments, the CRISPR gene-editing system for the treatment of gout comprises one or more sgRNAs targeting exon 9 of cTGFBR1.

In some embodiments, the CRISPR gene-editing system for the treatment of gout comprises one or more sgRNAs targeting exon 1 of eTGFBR1. In some embodiments, the CRISPR gene-editing system for the treatment of gout comprises one or more sgRNAs targeting exon 2 of eTGFBR1. In some embodiments, the CRISPR gene-editing system for the treatment of gout comprises one or more sgRNAs targeting exon 3 of eTGFBR1. In some embodiments, the CRISPR gene-editing system for the treatment of gout comprises one or more sgRNAs targeting exon 4 of eTGFBR1. In some embodiments, the CRISPR gene-editing system for the treatment of gout comprises one or more sgRNAs targeting exon 5 of eTGFBR1. In some embodiments, the CRISPR gene-editing system for the treatment of gout comprises one or more sgRNAs targeting exon 6 of eTGFBR1. In some embodiments, the CRISPR gene-editing system for the treatment of gout comprises one or more sgRNAs targeting exon 7 of eTGFBR1. In some embodiments, the CRISPR gene-editing system for the treatment of gout comprises one or more sgRNAs targeting exon 8 of eTGFBR1. In some embodiments, the CRISPR gene-editing system for the treatment of gout comprises one or more sgRNAs targeting exon 9 of eTGFBR1. In some embodiments, the CRISPR gene-editing system for the treatment of gout comprises one or more sgRNAs targeting exon 10 of eTGFBR1. In some embodiments, the CRISPR gene-editing system for the treatment of gout comprises one or more sgRNAs targeting exon 11 of eTGFBR1. In some embodiments, the CRISPR gene-editing system for the treatment of gout comprises one or more sgRNAs targeting exon 12 of eTGFBR1.

In some embodiments, the CRISPR gene-editing system for the treatment of gout comprises one or more sgRNAs targeting exon 1 of fTGFBR1. In some embodiments, the CRISPR gene-editing system for the treatment of gout comprises one or more sgRNAs targeting exon 2 of fTGFBR1. In some embodiments, the CRISPR gene-editing system for the treatment of gout comprises one or more sgRNAs targeting exon 3 of fTGFBR1. In some embodiments, the CRISPR gene-editing system for the treatment of gout comprises one or more sgRNAs targeting exon 4 of fTGFBR1. In some embodiments, the CRISPR gene-editing system for the treatment of gout comprises one or more sgRNAs targeting exon 5 of fTGFBR1. In some embodiments, the CRISPR gene-editing system for the treatment of gout comprises one or more sgRNAs targeting exon 6 of fTGFBR1. In some embodiments, the CRISPR gene-editing system for the treatment of gout comprises one or more sgRNAs targeting exon 7 of fTGFBR1. In some embodiments, the CRISPR gene-editing system for the treatment of gout comprises one or more sgRNAs targeting exon 8 of fTGFBR1. In some embodiments, the CRISPR gene-editing system for the treatment of gout comprises one or more sgRNAs targeting exon 9 of fTGFBR1. In some embodiments, the CRISPR gene-editing system for the treatment of gout comprises one or more sgRNAs targeting exon 10 of fTGFBR1. In some embodiments, the CRISPR gene-editing system for the treatment of gout comprises one or more sgRNAs targeting exon 11 of fTGFBR1.

In some embodiments, the CRISPR gene-editing system for the treatment of gout comprises one or more sgRNAs targeting TGFBR2. In some embodiments, the CRISPR gene-editing system for the treatment of gout comprises one or more sgRNAs targeting hTGFBR2. In some embodiments, the CRISPR gene-editing system for the treatment of gout comprises one or more sgRNAs targeting cTGFBR2. In some embodiments, the CRISPR gene-editing system for the treatment of gout comprises one or more sgRNAs targeting eTGFBR2. In some embodiments, the CRISPR gene-editing system for the treatment of gout comprises one or more sgRNAs targeting fTGFBR2.

In some embodiments, the CRISPR gene-editing system for the treatment of gout comprises one or more sgRNAs targeting exon 1 of hTGFBR2. In some embodiments, the CRISPR gene-editing system for the treatment of gout comprises one or more sgRNAs targeting exon 2 of hTGFBR2. In some embodiments, the CRISPR gene-editing system for the treatment of gout comprises one or more sgRNAs targeting exon 3 of hTGFBR2. In some embodiments, the CRISPR gene-editing system for the treatment of gout comprises one or more sgRNAs targeting exon 4 of hTGFBR2. In some embodiments, the CRISPR gene-editing system for the treatment of gout comprises one or more sgRNAs targeting exon 5 of hTGFBR2. In some embodiments, the CRISPR gene-editing system for the treatment of gout comprises one or more sgRNAs targeting exon 6 of hTGFBR2. In some embodiments, the CRISPR gene-editing system for the treatment of gout comprises one or more sgRNAs targeting exon 7 of hTGFBR2. In some embodiments, the CRISPR gene-editing system for the treatment of gout comprises one or more sgRNAs targeting exon 8 of hTGFBR2. In some embodiments, the CRISPR gene-editing system for the treatment of gout comprises one or more sgRNAs targeting exon 9 of hTGFBR2.

In some embodiments, the CRISPR gene-editing system for the treatment of gout comprises one or more sgRNAs targeting exon 1 of cTGFBR2. In some embodiments, the CRISPR gene-editing system for the treatment of gout comprises one or more sgRNAs targeting exon 2 of cTGFBR2. In some embodiments, the CRISPR gene-editing system for the treatment of gout comprises one or more sgRNAs targeting exon 3 of cTGFBR2. In some embodiments, the CRISPR gene-editing system for the treatment of gout comprises one or more sgRNAs targeting exon 4 of cTGFBR2. In some embodiments, the CRISPR gene-editing system for the treatment of gout comprises one or more sgRNAs targeting exon 5 of cTGFBR2. In some embodiments, the CRISPR gene-editing system for the treatment of gout comprises one or more sgRNAs targeting exon 6 of cTGFBR2. In some embodiments, the CRISPR gene-editing system for the treatment of gout comprises one or more sgRNAs targeting exon 7 of cTGFBR2. In some embodiments, the CRISPR gene-editing system for the treatment of gout comprises one or more sgRNAs targeting exon 8 of cTGFBR2.

In some embodiments, the CRISPR gene-editing system for the treatment of gout comprises one or more sgRNAs targeting exon 1 of eTGFBR2. In some embodiments, the CRISPR gene-editing system for the treatment of gout comprises one or more sgRNAs targeting exon 2 of eTGFBR2. In some embodiments, the CRISPR gene-editing system for the treatment of gout comprises one or more sgRNAs targeting exon 3 of eTGFBR2. In some embodiments, the CRISPR gene-editing system for the treatment of gout comprises one or more sgRNAs targeting exon 4 of eTGFBR2. In some embodiments, the CRISPR gene-editing system for the treatment of gout comprises one or more sgRNAs targeting exon 5 of eTGFBR2. In some embodiments, the CRISPR gene-editing system for the treatment of gout comprises one or more sgRNAs targeting exon 6 of eTGFBR2. In some embodiments, the CRISPR gene-editing system for the treatment of gout comprises one or more sgRNAs targeting exon 7 of eTGFBR2. In some embodiments, the CRISPR gene-editing system for the treatment of gout comprises one or more sgRNAs targeting exon 8 of eTGFBR2. In some embodiments, the CRISPR gene-editing system for the treatment of gout comprises one or more sgRNAs targeting exon 9 of eTGFBR2. In some embodiments, the CRISPR gene-editing system for the treatment of gout comprises one or more sgRNAs targeting exon 10 of eTGFBR2.

In some embodiments, the CRISPR gene-editing system for the treatment of gout comprises one or more sgRNAs targeting exon 1 of fTGFBR2. In some embodiments, the CRISPR gene-editing system for the treatment of gout comprises one or more sgRNAs targeting exon 2 of fTGFBR2. In some embodiments, the CRISPR gene-editing system for the treatment of gout comprises one or more sgRNAs targeting exon 3 of fTGFBR2. In some embodiments, the CRISPR gene-editing system for the treatment of gout comprises one or more sgRNAs targeting exon 4 of fTGFBR2. In some embodiments, the CRISPR gene-editing system for the treatment of gout comprises one or more sgRNAs targeting exon 5 of fTGFBR2. In some embodiments, the CRISPR gene-editing system for the treatment of gout comprises one or more sgRNAs targeting exon 6 of fTGFBR2. In some embodiments, the CRISPR gene-editing system for the treatment of gout comprises one or more sgRNAs targeting exon 7 of fTGFBR2. In some embodiments, the CRISPR gene-editing system for the treatment of gout comprises one or more sgRNAs targeting exon 8 of fTGFBR2.

In some embodiments, the CRISPR gene-editing system for the treatment of gout comprises one or more sgRNAs targeting IL6R. In some embodiments, the CRISPR gene-editing system for the treatment of gout comprises one or more sgRNAs targeting hIL6R. In some embodiments, the CRISPR gene-editing system for the treatment of gout comprises one or more sgRNAs targeting cIL6R. In some embodiments, the CRISPR gene-editing system for the treatment of gout comprises one or more sgRNAs targeting eIL6R. In some embodiments, the CRISPR gene-editing system for the treatment of gout comprises one or more sgRNAs targeting fIL6R.

In some embodiments, the CRISPR gene-editing system for the treatment of gout comprises one or more sgRNAs targeting exon 1 of hIL6R. In some embodiments, the CRISPR gene-editing system for the treatment of gout comprises one or more sgRNAs targeting exon 2 of hIL6R. In some embodiments, the CRISPR gene-editing system for the treatment of gout comprises one or more sgRNAs targeting exon 3 of hIL6R. In some embodiments, the CRISPR gene-editing system for the treatment of gout comprises one or more sgRNAs targeting exon 4 of hIL6R. In some embodiments, the CRISPR gene-editing system for the treatment of gout comprises one or more sgRNAs targeting exon 5 of hIL6R. In some embodiments, the CRISPR gene-editing system for the treatment of gout comprises one or more sgRNAs targeting exon 6 of hIL6R. In some embodiments, the CRISPR gene-editing system for the treatment of gout comprises one or more sgRNAs targeting exon 7 of hIL6R. In some embodiments, the CRISPR gene-editing system for the treatment of gout comprises one or more sgRNAs targeting exon 8 of hIL6R. In some embodiments, the CRISPR gene-editing system for the treatment of gout comprises one or more sgRNAs targeting exon 9 of hIL6R. In some embodiments, the CRISPR gene-editing system for the treatment of gout comprises one or more sgRNAs targeting exon 10 of hIL6R. In some embodiments, the CRISPR gene-editing system for the treatment of gout comprises one or more sgRNAs targeting exon 11 of hIL6R In some embodiments, the CRISPR gene-editing system for the treatment of gout comprises one or more sgRNAs targeting exon 12 of hIL6R. In some embodiments, the CRISPR gene-editing system for the treatment of gout comprises one or more sgRNAs targeting exon 13 of hIL6R. In some embodiments, the CRISPR gene-editing system for the treatment of gout comprises one or more sgRNAs targeting exon 14 of hIL6R. In some embodiments, the CRISPR gene-editing system for the treatment of gout comprises one or more sgRNAs targeting exon 15 of hIL6R.

In some embodiments, the CRISPR gene-editing system for the treatment of gout comprises one or more sgRNAs targeting exon 1 of cIL6R. In some embodiments, the CRISPR gene-editing system for the treatment of gout comprises one or more sgRNAs targeting exon 2 of cIL6R. In some embodiments, the CRISPR gene-editing system for the treatment of gout comprises one or more sgRNAs targeting exon 3 of cIL6R. In some embodiments, the CRISPR gene-editing system for the treatment of gout comprises one or more sgRNAs targeting exon 4 of cIL6R. In some embodiments, the CRISPR gene-editing system for the treatment of gout comprises one or more sgRNAs targeting exon 5 of cIL6R. In some embodiments, the CRISPR gene-editing system for the treatment of gout comprises one or more sgRNAs targeting exon 6 of cIL6R. In some embodiments, the CRISPR gene-editing system for the treatment of gout comprises one or more sgRNAs targeting exon 7 of cIL6R. In some embodiments, the CRISPR gene-editing system for the treatment of gout comprises one or more sgRNAs targeting exon 8 of cIL6R. In some embodiments, the CRISPR gene-editing system for the treatment of gout comprises one or more sgRNAs targeting exon 9 of cIL6R. In some embodiments, the CRISPR gene-editing system for the treatment of gout comprises one or more sgRNAs targeting exon 10 of cIL6R. In some embodiments, the CRISPR gene-editing system for the treatment of gout comprises one or more sgRNAs targeting exon 11 of cIL6R. In some embodiments, the CRISPR gene-editing system for the treatment of gout comprises one or more sgRNAs targeting exon 12 of cIL6R. In some embodiments, the CRISPR gene-editing system for the treatment of gout comprises one or more sgRNAs targeting exon 13 of cIL6R.

In some embodiments, the CRISPR gene-editing system for the treatment of gout comprises one or more sgRNAs targeting exon 1 of eIL6R. In some embodiments, the CRISPR gene-editing system for the treatment of gout comprises one or more sgRNAs targeting exon 2 of eIL6R. In some embodiments, the CRISPR gene-editing system for the treatment of gout comprises one or more sgRNAs targeting exon 3 of eIL6R. In some embodiments, the CRISPR gene-editing system for the treatment of gout comprises one or more sgRNAs targeting exon 4 of eIL6R. In some embodiments, the CRISPR gene-editing system for the treatment of gout comprises one or more sgRNAs targeting exon 5 of eIL6R. In some embodiments, the CRISPR gene-editing system for the treatment of gout comprises one or more sgRNAs targeting exon 6 of eIL6R. In some embodiments, the CRISPR gene-editing system for the treatment of gout comprises one or more sgRNAs targeting exon 7 of eIL6R. In some embodiments, the CRISPR gene-editing system for the treatment of gout comprises one or more sgRNAs targeting exon 8 of eIL6R. In some embodiments, the CRISPR gene-editing system for the treatment of gout comprises one or more sgRNAs targeting exon 9 of eIL6R. In some embodiments, the CRISPR gene-editing system for the treatment of gout comprises one or more sgRNAs targeting exon 10 of eIL6R. In some embodiments, the CRISPR gene-editing system for the treatment of gout comprises one or more sgRNAs targeting exon 11 of eIL6R.

In some embodiments, the CRISPR gene-editing system for the treatment of gout comprises one or more sgRNAs targeting exon 1 of fIL6R. In some embodiments, the CRISPR gene-editing system for the treatment of gout comprises one or more sgRNAs targeting exon 2 of fIL6R. In some embodiments, the CRISPR gene-editing system for the treatment of gout comprises one or more sgRNAs targeting exon 3 of fIL6R. In some embodiments, the CRISPR gene-editing system for the treatment of gout comprises one or more sgRNAs targeting exon 4 of fIL6R. In some embodiments, the CRISPR gene-editing system for the treatment of gout comprises one or more sgRNAs targeting exon 5 of fIL6R. In some embodiments, the CRISPR gene-editing system for the treatment of gout comprises one or more sgRNAs targeting exon 6 of fIL6R. In some embodiments, the CRISPR gene-editing system for the treatment of gout comprises one or more sgRNAs targeting exon 7 of fIL6R. In some embodiments, the CRISPR gene-editing system for the treatment of gout comprises one or more sgRNAs targeting exon 8 of fIL6R. In some embodiments, the CRISPR gene-editing system for the treatment of gout comprises one or more sgRNAs targeting exon 9 of fIL6R. In some embodiments, the CRISPR gene-editing system for the treatment of gout comprises one or more sgRNAs targeting exon 10 of fIL6R.

In some embodiments, the CRISPR gene-editing system for the treatment of gout comprises one or more sgRNAs targeting IL6ST. In some embodiments, the CRISPR gene-editing system for the treatment of gout comprises one or more sgRNAs targeting hIL6ST. In some embodiments, the CRISPR gene-editing system for the treatment of gout comprises one or more sgRNAs targeting cIL6ST. In some embodiments, the CRISPR gene-editing system for the treatment of gout comprises one or more sgRNAs targeting eIL6ST. In some embodiments, the CRISPR gene-editing system for the treatment of gout comprises one or more sgRNAs targeting fIL6ST.

In some embodiments, the CRISPR gene-editing system for the treatment of gout comprises one or more sgRNAs targeting exon 1 of hIL6ST. In some embodiments, the CRISPR gene-editing system for the treatment of gout comprises one or more sgRNAs targeting exon 2 of hIL6ST. In some embodiments, the CRISPR gene-editing system for the treatment of gout comprises one or more sgRNAs targeting exon 3 of hIL6ST. In some embodiments, the CRISPR gene-editing system for the treatment of gout comprises one or more sgRNAs targeting exon 4 of hIL6ST. In some embodiments, the CRISPR gene-editing system for the treatment of gout comprises one or more sgRNAs targeting exon 5 of hIL6ST. In some embodiments, the CRISPR gene-editing system for the treatment of gout comprises one or more sgRNAs targeting exon 6 of hIL6ST. In some embodiments, the CRISPR gene-editing system for the treatment of gout comprises one or more sgRNAs targeting exon 7 of hIL6ST. In some embodiments, the CRISPR gene-editing system for the treatment of gout comprises one or more sgRNAs targeting exon 8 of hIL6ST. In some embodiments, the CRISPR gene-editing system for the treatment of gout comprises one or more sgRNAs targeting exon 9 of hIL6ST. In some embodiments, the CRISPR gene-editing system for the treatment of gout comprises one or more sgRNAs targeting exon 10 of hIL6ST. In some embodiments, the CRISPR gene-editing system for the treatment of gout comprises one or more sgRNAs targeting exon 11 of hIL6ST. In some embodiments, the CRISPR gene-editing system for the treatment of gout comprises one or more sgRNAs targeting exon 12 of hIL6ST. In some embodiments, the CRISPR gene-editing system for the treatment of gout comprises one or more sgRNAs targeting exon 13 of hIL6ST. In some embodiments, the CRISPR gene-editing system for the treatment of gout comprises one or more sgRNAs targeting exon 14 of hIL6ST. In some embodiments, the CRISPR gene-editing system for the treatment of gout comprises one or more sgRNAs targeting exon 15 of hIL6ST. In some embodiments, the CRISPR gene-editing system for the treatment of gout comprises one or more sgRNAs targeting exon 16 of hIL6ST. In some embodiments, the CRISPR gene-editing system for the treatment of gout comprises one or more sgRNAs targeting exon 17 of hIL6ST. In some embodiments, the CRISPR gene-editing system for the treatment of gout comprises one or more sgRNAs targeting exon 18 of hIL6ST. In some embodiments, the CRISPR gene-editing system for the treatment of gout comprises one or more sgRNAs targeting exon 19 of hIL6ST.

In some embodiments, the CRISPR gene-editing system for the treatment of gout comprises one or more sgRNAs targeting exon 1 of cIL6ST. In some embodiments, the CRISPR gene-editing system for the treatment of gout comprises one or more sgRNAs targeting exon 2 of cIL6ST. In some embodiments, the CRISPR gene-editing system for the treatment of gout comprises one or more sgRNAs targeting exon 3 of cIL6ST. In some embodiments, the CRISPR gene-editing system for the treatment of gout comprises one or more sgRNAs targeting exon 4 of cIL6ST. In some embodiments, the CRISPR gene-editing system for the treatment of gout comprises one or more sgRNAs targeting exon 5 of cIL6ST. In some embodiments, the CRISPR gene-editing system for the treatment of gout comprises one or more sgRNAs targeting exon 6 of cIL6ST. In some embodiments, the CRISPR gene-editing system for the treatment of gout comprises one or more sgRNAs targeting exon 7 of cIL6ST. In some embodiments, the CRISPR gene-editing system for the treatment of gout comprises one or more sgRNAs targeting exon 8 of cIL6ST. In some embodiments, the CRISPR gene-editing system for the treatment of gout comprises one or more sgRNAs targeting exon 9 of cIL6ST. In some embodiments, the CRISPR gene-editing system for the treatment of gout comprises one or more sgRNAs targeting exon 10 of cIL6ST. In some embodiments, the CRISPR gene-editing system for the treatment of gout comprises one or more sgRNAs targeting exon 11 of cIL6ST In some embodiments, the CRISPR gene-editing system for the treatment of gout comprises one or more sgRNAs targeting exon 12 of cIL6ST. In some embodiments, the CRISPR gene-editing system for the treatment of gout comprises one or more sgRNAs targeting exon 13 of cIL6ST. In some embodiments, the CRISPR gene-editing system for the treatment of gout comprises one or more sgRNAs targeting exon 14 of cIL6ST. In some embodiments, the CRISPR gene-editing system for the treatment of gout comprises one or more sgRNAs targeting exon 15 of cIL6ST. In some embodiments, the CRISPR gene-editing system for the treatment of gout comprises one or more sgRNAs targeting exon 16 of cIL6ST. In some embodiments, the CRISPR gene-editing system for the treatment of gout comprises one or more sgRNAs targeting exon 17 of cIL6ST. In some embodiments, the CRISPR gene-editing system for the treatment of gout comprises one or more sgRNAs targeting exon 18 of cIL6ST.

In some embodiments, the CRISPR gene-editing system for the treatment of gout comprises one or more sgRNAs targeting exon 1 of eIL6ST. In some embodiments, the CRISPR gene-editing system for the treatment of gout comprises one or more sgRNAs targeting exon 2 of eIL6ST. In some embodiments, the CRISPR gene-editing system for the treatment of gout comprises one or more sgRNAs targeting exon 3 of eIL6ST. In some embodiments, the CRISPR gene-editing system for the treatment of gout comprises one or more sgRNAs targeting exon 4 of eIL6ST. In some embodiments, the CRISPR gene-editing system for the treatment of gout comprises one or more sgRNAs targeting exon 5 of eIL6ST. In some embodiments, the CRISPR gene-editing system for the treatment of gout comprises one or more sgRNAs targeting exon 6 of eIL6ST. In some embodiments, the CRISPR gene-editing system for the treatment of gout comprises one or more sgRNAs targeting exon 7 of eIL6ST. In some embodiments, the CRISPR gene-editing system for the treatment of gout comprises one or more sgRNAs targeting exon 8 of eIL6ST. In some embodiments, the CRISPR gene-editing system for the treatment of gout comprises one or more sgRNAs targeting exon 9 of eIL6ST. In some embodiments, the CRISPR gene-editing system for the treatment of gout comprises one or more sgRNAs targeting exon 10 of eIL6ST. In some embodiments, the CRISPR gene-editing system for the treatment of gout comprises one or more sgRNAs targeting exon 11 of eIL6ST In some embodiments, the CRISPR gene-editing system for the treatment of gout comprises one or more sgRNAs targeting exon 12 of eIL6ST. In some embodiments, the CRISPR gene-editing system for the treatment of gout comprises one or more sgRNAs targeting exon 13 of eIL6ST. In some embodiments, the CRISPR gene-editing system for the treatment of gout comprises one or more sgRNAs targeting exon 14 of eIL6ST. In some embodiments, the CRISPR gene-editing system for the treatment of gout comprises one or more sgRNAs targeting exon 15 of eIL6ST. In some embodiments, the CRISPR gene-editing system for the treatment of gout comprises one or more sgRNAs targeting exon 16 of eIL6ST. In some embodiments, the CRISPR gene-editing system for the treatment of gout comprises one or more sgRNAs targeting exon 17 of eIL6ST. In some embodiments, the CRISPR gene-editing system for the treatment of gout comprises one or more sgRNAs targeting exon 18 of eIL6ST.

In some embodiments, the CRISPR gene-editing system for the treatment of gout comprises one or more sgRNAs targeting exon 1 of fIL6ST. In some embodiments, the CRISPR gene-editing system for the treatment of gout comprises one or more sgRNAs targeting exon 2 of fIL6ST. In some embodiments, the CRISPR gene-editing system for the treatment of gout comprises one or more sgRNAs targeting exon 3 of fIL6ST. In some embodiments, the CRISPR gene-editing system for the treatment of gout comprises one or more sgRNAs targeting exon 4 of fIL6ST. In some embodiments, the CRISPR gene-editing system for the treatment of gout comprises one or more sgRNAs targeting exon 5 of fIL6ST. In some embodiments, the CRISPR gene-editing system for the treatment of gout comprises one or more sgRNAs targeting exon 6 of fIL6ST. In some embodiments, the CRISPR gene-editing system for the treatment of gout comprises one or more sgRNAs targeting exon 7 of fIL6ST. In some embodiments, the CRISPR gene-editing system for the treatment of gout comprises one or more sgRNAs targeting exon 8 of fIL6ST. In some embodiments, the CRISPR gene-editing system for the treatment of gout comprises one or more sgRNAs targeting exon 9 of fIL6ST. In some embodiments, the CRISPR gene-editing system for the treatment of gout comprises one or more sgRNAs targeting exon 10 of fIL6ST. In some embodiments, the CRISPR gene-editing system for the treatment of gout comprises one or more sgRNAs targeting exon 11 of fIL6ST In some embodiments, the CRISPR gene-editing system for the treatment of gout comprises one or more sgRNAs targeting exon 12 of fIL6ST. In some embodiments, the CRISPR gene-editing system for the treatment of gout comprises one or more sgRNAs targeting exon 13 of fIL6ST. In some embodiments, the CRISPR gene-editing system for the treatment of gout comprises one or more sgRNAs targeting exon 14 of fIL6ST. In some embodiments, the CRISPR gene-editing system for the treatment of gout comprises one or more sgRNAs targeting exon 15 of fIL6ST. In some embodiments, the CRISPR gene-editing system for the treatment of gout comprises one or more sgRNAs targeting exon 16 of fIL6ST. In some embodiments, the CRISPR gene-editing system for the treatment of gout comprises one or more sgRNAs targeting exon 17 of fIL6ST. In some embodiments, the CRISPR gene-editing system for the treatment of gout comprises one or more sgRNAs targeting exon 18 of fIL6ST. In some embodiments, the CRISPR gene-editing system for the treatment of gout comprises one or more sgRNAs targeting exon 19 of fIL6ST. In some embodiments, the CRISPR gene-editing system for the treatment of gout comprises one or more sgRNAs targeting exon 20 of fIL6ST.

In some embodiments, the CRISPR gene-editing system for the treatment of gout comprises one or more sgRNAs targeting TNFRSF1A. In some embodiments, the CRISPR gene-editing system for the treatment of gout comprises one or more sgRNAs targeting hTNFRSF1A. In some embodiments, the CRISPR gene-editing system for the treatment of gout comprises one or more sgRNAs targeting cTNFRSF1A. In some embodiments, the CRISPR gene-editing system for the treatment of gout comprises one or more sgRNAs targeting eTNFRSF1A. In some embodiments, the CRISPR gene-editing system for the treatment of gout comprises one or more sgRNAs targeting fTNFRSF1A.

In some embodiments, the CRISPR gene-editing system for the treatment of gout comprises one or more sgRNAs targeting exon 1 of hTNFRSF1A. In some embodiments, the CRISPR gene-editing system for the treatment of gout comprises one or more sgRNAs targeting exon 2 of hTNFRSF1A. In some embodiments, the CRISPR gene-editing system for the treatment of gout comprises one or more sgRNAs targeting exon 3 of hTNFRSF1A. In some embodiments, the CRISPR gene-editing system for the treatment of gout comprises one or more sgRNAs targeting exon 4 of hTNFRSF1A. In some embodiments, the CRISPR gene-editing system for the treatment of gout comprises one or more sgRNAs targeting exon 5 of hTNFRSF1A. In some embodiments, the CRISPR gene-editing system for the treatment of gout comprises one or more sgRNAs targeting exon 6 of hTNFRSF1A. In some embodiments, the CRISPR gene-editing system for the treatment of gout comprises one or more sgRNAs targeting exon 7 of hTNFRSF1A. In some embodiments, the CRISPR gene-editing system for the treatment of gout comprises one or more sgRNAs targeting exon 8 of hTNFRSF1A. In some embodiments, the CRISPR gene-editing system for the treatment of gout comprises one or more sgRNAs targeting exon 9 of hTNFRSF1A. In some embodiments, the CRISPR gene-editing system for the treatment of gout comprises one or more sgRNAs targeting exon 10 of hTNFRSF1A. In some embodiments, the CRISPR gene-editing system for the treatment of gout comprises one or more sgRNAs targeting exon 11 of hTNFRSF1A.

In some embodiments, the CRISPR gene-editing system for the treatment of gout comprises one or more sgRNAs targeting exon 1 of cTNFRSF1A. In some embodiments, the CRISPR gene-editing system for the treatment of gout comprises one or more sgRNAs targeting exon 2 of cTNFRSF1A. In some embodiments, the CRISPR gene-editing system for the treatment of gout comprises one or more sgRNAs targeting exon 3 of cTNFRSF1A. In some embodiments, the CRISPR gene-editing system for the treatment of gout comprises one or more sgRNAs targeting exon 4 of cTNFRSF1A. In some embodiments, the CRISPR gene-editing system for the treatment of gout comprises one or more sgRNAs targeting exon 5 of cTNFRSF1A. In some embodiments, the CRISPR gene-editing system for the treatment of gout comprises one or more sgRNAs targeting exon 6 of cTNFRSF1A. In some embodiments, the CRISPR gene-editing system for the treatment of gout comprises one or more sgRNAs targeting exon 7 of cTNFRSF1A. In some embodiments, the CRISPR gene-editing system for the treatment of gout comprises one or more sgRNAs targeting exon 8 of cTNFRSF1A. In some embodiments, the CRISPR gene-editing system for the treatment of gout comprises one or more sgRNAs targeting exon 9 of cTNFRSF1A. In some embodiments, the CRISPR gene-editing system for the treatment of gout comprises one or more sgRNAs targeting exon 10 of cTNFRSF1A. In some embodiments, the CRISPR gene-editing system for the treatment of gout comprises one or more sgRNAs targeting exon 11 of cTNFRSF1A.

In some embodiments, the CRISPR gene-editing system for the treatment of gout comprises one or more sgRNAs targeting exon 1 of eTNFRSF1A. In some embodiments, the CRISPR gene-editing system for the treatment of gout comprises one or more sgRNAs targeting exon 2 of eTNFRSF1A. In some embodiments, the CRISPR gene-editing system for the treatment of gout comprises one or more sgRNAs targeting exon 3 of eTNFRSF1A. In some embodiments, the CRISPR gene-editing system for the treatment of gout comprises one or more sgRNAs targeting exon 4 of eTNFRSF1A. In some embodiments, the CRISPR gene-editing system for the treatment of gout comprises one or more sgRNAs targeting exon 5 of eTNFRSF1A. In some embodiments, the CRISPR gene-editing system for the treatment of gout comprises one or more sgRNAs targeting exon 6 of eTNFRSF1A. In some embodiments, the CRISPR gene-editing system for the treatment of gout comprises one or more sgRNAs targeting exon 7 of eTNFRSF1A. In some embodiments, the CRISPR gene-editing system for the treatment of gout comprises one or more sgRNAs targeting exon 8 of eTNFRSF1A. In some embodiments, the CRISPR gene-editing system for the treatment of gout comprises one or more sgRNAs targeting exon 9 of eTNFRSF1A. In some embodiments, the CRISPR gene-editing system for the treatment of gout comprises one or more sgRNAs targeting exon 10 of eTNFRSF1A.

In some embodiments, the CRISPR gene-editing system for the treatment of gout comprises one or more sgRNAs targeting exon 1 of fTNFRSF1A. In some embodiments, the CRISPR gene-editing system for the treatment of gout comprises one or more sgRNAs targeting exon 2 of fTNFRSF1A. In some embodiments, the CRISPR gene-editing system for the treatment of gout comprises one or more sgRNAs targeting exon 3 of fTNFRSF1A. In some embodiments, the CRISPR gene-editing system for the treatment of gout comprises one or more sgRNAs targeting exon 4 of fINFRSF1A. In some embodiments, the CRISPR gene-editing system for the treatment of gout comprises one or more sgRNAs targeting exon 5 of fTNFRSF1A. In some embodiments, the CRISPR gene-editing system for the treatment of gout comprises one or more sgRNAs targeting exon 6 of fTNFRSF1A. In some embodiments, the CRISPR gene-editing system for the treatment of gout comprises one or more sgRNAs targeting exon 7 of fTNFRSF1A. In some embodiments, the CRISPR gene-editing system for the treatment of gout comprises one or more sgRNAs targeting exon 8 of fTNFRSF1A. In some embodiments, the CRISPR gene-editing system for the treatment of gout comprises one or more sgRNAs targeting exon 9 of fTNFRSF1A. In some embodiments, the CRISPR gene-editing system for the treatment of gout comprises one or more sgRNAs targeting exon 10 of fTNFRSF1A.

In some embodiments, the CRISPR gene-editing system for the treatment of gout comprises one or more sgRNAs targeting TNFRSF1B. In some embodiments, the CRISPR gene-editing system for the treatment of gout comprises one or more sgRNAs targeting hTNFRSF1B. In some embodiments, the CRISPR gene-editing system for the treatment of gout comprises one or more sgRNAs targeting cTNFRSF1B. In some embodiments, the CRISPR gene-editing system for the treatment of gout comprises one or more sgRNAs targeting eTNFRSF1B. In some embodiments, the CRISPR gene-editing system for the treatment of gout comprises one or more sgRNAs targeting fTNFRSF1B.

In some embodiments, the CRISPR gene-editing system for the treatment of gout comprises one or more sgRNAs targeting exon 1 of hTNFRSF1B. In some embodiments, the CRISPR gene-editing system for the treatment of gout comprises one or more sgRNAs targeting exon 2 of hTNFRSF1B. In some embodiments, the CRISPR gene-editing system for the treatment of gout comprises one or more sgRNAs targeting exon 3 of hTNFRSF1B. In some embodiments, the CRISPR gene-editing system for the treatment of gout comprises one or more sgRNAs targeting exon 4 of hTNFRSF1B. In some embodiments, the CRISPR gene-editing system for the treatment of gout comprises one or more sgRNAs targeting exon 5 of hTNFRSF1B. In some embodiments, the CRISPR gene-editing system for the treatment of gout comprises one or more sgRNAs targeting exon 6 of hTNFRSF1B. In some embodiments, the CRISPR gene-editing system for the treatment of gout comprises one or more sgRNAs targeting exon 7 of hTNFRSF1B. In some embodiments, the CRISPR gene-editing system for the treatment of gout comprises one or more sgRNAs targeting exon 8 of hTNFRSF1B. In some embodiments, the CRISPR gene-editing system for the treatment of gout comprises one or more sgRNAs targeting exon 9 of hTNFRSF1B. In some embodiments, the CRISPR gene-editing system for the treatment of gout comprises one or more sgRNAs targeting exon 10 of hTNFRSF1B. In some embodiments, the CRISPR gene-editing system for the treatment of gout comprises one or more sgRNAs targeting exon 11 of hTNFRSF1B In some embodiments, the CRISPR gene-editing system for the treatment of gout comprises one or more sgRNAs targeting exon 12 of hTNFRSF1B. In some embodiments, the CRISPR gene-editing system for the treatment of gout comprises one or more sgRNAs targeting exon 13 of hTNFRSF1B.

In some embodiments, the CRISPR gene-editing system for the treatment of gout comprises one or more sgRNAs targeting exon 1 of cTNFRSF1B. In some embodiments, the CRISPR gene-editing system for the treatment of gout comprises one or more sgRNAs targeting exon 2 of cTNFRSF1B. In some embodiments, the CRISPR gene-editing system for the treatment of gout comprises one or more sgRNAs targeting exon 3 of cTNFRSF1B. In some embodiments, the CRISPR gene-editing system for the treatment of gout comprises one or more sgRNAs targeting exon 4 of cTNFRSF1B. In some embodiments, the CRISPR gene-editing system for the treatment of gout comprises one or more sgRNAs targeting exon 5 of cTNFRSF1B. In some embodiments, the CRISPR gene-editing system for the treatment of gout comprises one or more sgRNAs targeting exon 6 of cTNFRSF1B. In some embodiments, the CRISPR gene-editing system for the treatment of gout comprises one or more sgRNAs targeting exon 7 of cTNFRSF1B. In some embodiments, the CRISPR gene-editing system for the treatment of gout comprises one or more sgRNAs targeting exon 8 of cTNFRSF1B. In some embodiments, the CRISPR gene-editing system for the treatment of gout comprises one or more sgRNAs targeting exon 9 of cTNFRSF1B. In some embodiments, the CRISPR gene-editing system for the treatment of gout comprises one or more sgRNAs targeting exon 10 of cTNFRSF1B. In some embodiments, the CRISPR gene-editing system for the treatment of gout comprises one or more sgRNAs targeting exon 11 of cTNFRSF1B.

In some embodiments, the CRISPR gene-editing system for the treatment of gout comprises one or more sgRNAs targeting exon 1 of eTNFRSF1B. In some embodiments, the CRISPR gene-editing system for the treatment of gout comprises one or more sgRNAs targeting exon 2 of eTNFRSF1B. In some embodiments, the CRISPR gene-editing system for the treatment of gout comprises one or more sgRNAs targeting exon 3 of eTNFRSF1B. In some embodiments, the CRISPR gene-editing system for the treatment of gout comprises one or more sgRNAs targeting exon 4 of eTNFRSF1B. In some embodiments, the CRISPR gene-editing system for the treatment of gout comprises one or more sgRNAs targeting exon 5 of eTNFRSF1B. In some embodiments, the CRISPR gene-editing system for the treatment of gout comprises one or more sgRNAs targeting exon 6 of eTNFRSF1B. In some embodiments, the CRISPR gene-editing system for the treatment of gout comprises one or more sgRNAs targeting exon 7 of eTNFRSF1B. In some embodiments, the CRISPR gene-editing system for the treatment of gout comprises one or more sgRNAs targeting exon 8 of eTNFRSF1B. In some embodiments, the CRISPR gene-editing system for the treatment of gout comprises one or more sgRNAs targeting exon 9 of eTNFRSF1B. In some embodiments, the CRISPR gene-editing system for the treatment of gout comprises one or more sgRNAs targeting exon 10 of eTNFRSF1B. In some embodiments, the CRISPR gene-editing system for the treatment of gout comprises one or more sgRNAs targeting exon 11 of eTNFRSF1B.

In some embodiments, the CRISPR gene-editing system for the treatment of gout comprises one or more sgRNAs targeting exon 1 of fTNFRSF1B. In some embodiments, the CRISPR gene-editing system for the treatment of gout comprises one or more sgRNAs targeting exon 2 of fTNFRSF1B. In some embodiments, the CRISPR gene-editing system for the treatment of gout comprises one or more sgRNAs targeting exon 3 of fTNFRSF1B. In some embodiments, the CRISPR gene-editing system for the treatment of gout comprises one or more sgRNAs targeting exon 4 of fTNFRSF1B. In some embodiments, the CRISPR gene-editing system for the treatment of gout comprises one or more sgRNAs targeting exon 5 of fTNFRSF1B. In some embodiments, the CRISPR gene-editing system for the treatment of gout comprises one or more sgRNAs targeting exon 6 of fTNFRSF1B. In some embodiments, the CRISPR gene-editing system for the treatment of gout comprises one or more sgRNAs targeting exon 7 of fTNFRSF1B. In some embodiments, the CRISPR gene-editing system for the treatment of gout comprises one or more sgRNAs targeting exon 8 of fTNFRSF1B. In some embodiments, the CRISPR gene-editing system for the treatment of gout comprises one or more sgRNAs targeting exon 9 of fTNFRSF1B. In some embodiments, the CRISPR gene-editing system for the treatment of gout comprises one or more sgRNAs targeting exon 10 of fTNFRSF1B.

In some embodiments, the CRISPR gene-editing system for the treatment of gout comprises one or more sgRNAs targeting TNFRSF3. In some embodiments, the CRISPR gene-editing system for the treatment of gout comprises one or more sgRNAs targeting hTNFRSF3. In some embodiments, the CRISPR gene-editing system for the treatment of gout comprises one or more sgRNAs targeting cTNFRSF3. In some embodiments, the CRISPR gene-editing system for the treatment of gout comprises one or more sgRNAs targeting eTNFRSF3. In some embodiments, the CRISPR gene-editing system for the treatment of gout comprises one or more sgRNAs targeting fTNFRSF3.

In some embodiments, the CRISPR gene-editing system for the treatment of gout comprises one or more sgRNAs targeting exon 1 of hTNFRSF3. In some embodiments, the CRISPR gene-editing system for the treatment of gout comprises one or more sgRNAs targeting exon 2 of hTNFRSF3. In some embodiments, the CRISPR gene-editing system for the treatment of gout comprises one or more sgRNAs targeting exon 3 of hTNFRSF3. In some embodiments, the CRISPR gene-editing system for the treatment of gout comprises one or more sgRNAs targeting exon 4 of hTNFRSF3. In some embodiments, the CRISPR gene-editing system for the treatment of gout comprises one or more sgRNAs targeting exon 5 of hTNFRSF3. In some embodiments, the CRISPR gene-editing system for the treatment of gout comprises one or more sgRNAs targeting exon 6 of hTNFRSF3. In some embodiments, the CRISPR gene-editing system for the treatment of gout comprises one or more sgRNAs targeting exon 7 of hTNFRSF3. In some embodiments, the CRISPR gene-editing system for the treatment of gout comprises one or more sgRNAs targeting exon 8 of hTNFRSF3. In some embodiments, the CRISPR gene-editing system for the treatment of gout comprises one or more sgRNAs targeting exon 9 of hTNFRSF3. In some embodiments, the CRISPR gene-editing system for the treatment of gout comprises one or more sgRNAs targeting exon 10 of hTNFRSF3. In some embodiments, the CRISPR gene-editing system for the treatment of gout comprises one or more sgRNAs targeting exon 11 of hTNFRSF3. In some embodiments, the CRISPR gene-editing system for the treatment of gout comprises one or more sgRNAs targeting exon 12 of hTNFRSF3.

In some embodiments, the CRISPR gene-editing system for the treatment of gout comprises one or more sgRNAs targeting exon 1 of cTNFRSF3. In some embodiments, the CRISPR gene-editing system for the treatment of gout comprises one or more sgRNAs targeting exon 2 of cTNFRSF3. In some embodiments, the CRISPR gene-editing system for the treatment of gout comprises one or more sgRNAs targeting exon 3 of cTNFRSF3. In some embodiments, the CRISPR gene-editing system for the treatment of gout comprises one or more sgRNAs targeting exon 4 of cTNFRSF3. In some embodiments, the CRISPR gene-editing system for the treatment of gout comprises one or more sgRNAs targeting exon 5 of cTNFRSF3. In some embodiments, the CRISPR gene-editing system for the treatment of gout comprises one or more sgRNAs targeting exon 6 of cTNFRSF3. In some embodiments, the CRISPR gene-editing system for the treatment of gout comprises one or more sgRNAs targeting exon 7 of cTNFRSF3. In some embodiments, the CRISPR gene-editing system for the treatment of gout comprises one or more sgRNAs targeting exon 8 of cTNFRSF3. In some embodiments, the CRISPR gene-editing system for the treatment of gout comprises one or more sgRNAs targeting exon 9 of cTNFRSF3. In some embodiments, the CRISPR gene-editing system for the treatment of gout comprises one or more sgRNAs targeting exon 10 of cTNFRSF3.

In some embodiments, the CRISPR gene-editing system for the treatment of gout comprises one or more sgRNAs targeting exon 1 of eTNFRSF3. In some embodiments, the CRISPR gene-editing system for the treatment of gout comprises one or more sgRNAs targeting exon 2 of eTNFRSF3. In some embodiments, the CRISPR gene-editing system for the treatment of gout comprises one or more sgRNAs targeting exon 3 of eTNFRSF3. In some embodiments, the CRISPR gene-editing system for the treatment of gout comprises one or more sgRNAs targeting exon 4 of eTNFRSF3. In some embodiments, the CRISPR gene-editing system for the treatment of gout comprises one or more sgRNAs targeting exon 5 of eTNFRSF3. In some embodiments, the CRISPR gene-editing system for the treatment of gout comprises one or more sgRNAs targeting exon 6 of eTNFRSF3. In some embodiments, the CRISPR gene-editing system for the treatment of gout comprises one or more sgRNAs targeting exon 7 of eTNFRSF3. In some embodiments, the CRISPR gene-editing system for the treatment of gout comprises one or more sgRNAs targeting exon 8 of eTNFRSF3. In some embodiments, the CRISPR gene-editing system for the treatment of gout comprises one or more sgRNAs targeting exon 9 of eTNFRSF3. In some embodiments, the CRISPR gene-editing system for the treatment of gout comprises one or more sgRNAs targeting exon 10 of eTNFRSF3.

In some embodiments, the CRISPR gene-editing system for the treatment of gout comprises one or more sgRNAs targeting exon 1 of fTNFRSF3. In some embodiments, the CRISPR gene-editing system for the treatment of gout comprises one or more sgRNAs targeting exon 2 of fTNFRSF3. In some embodiments, the CRISPR gene-editing system for the treatment of gout comprises one or more sgRNAs targeting exon 3 of fTNFRSF3. In some embodiments, the CRISPR gene-editing system for the treatment of gout comprises one or more sgRNAs targeting exon 4 of fTNFRSF3. In some embodiments, the CRISPR gene-editing system for the treatment of gout comprises one or more sgRNAs targeting exon 5 of fTNFRSF3. In some embodiments, the CRISPR gene-editing system for the treatment of gout comprises one or more sgRNAs targeting exon 6 of fTNFRSF3. In some embodiments, the CRISPR gene-editing system for the treatment of gout comprises one or more sgRNAs targeting exon 7 of fTNFRSF3. In some embodiments, the CRISPR gene-editing system for the treatment of gout comprises one or more sgRNAs targeting exon 8 of fTNFRSF3. In some embodiments, the CRISPR gene-editing system for the treatment of gout comprises one or more sgRNAs targeting exon 9 of fTNFRSF3. In some embodiments, the CRISPR gene-editing system for the treatment of gout comprises one or more sgRNAs targeting exon 10 of fTNFRSF3.

In some embodiments, the CRISPR gene-editing system for the treatment of gout comprises one or more sgRNAs targeting TNFRSF4. In some embodiments, the CRISPR gene-editing system for the treatment of gout comprises one or more sgRNAs targeting hTNFRSF4. In some embodiments, the CRISPR gene-editing system for the treatment of gout comprises one or more sgRNAs targeting cTNFRSF4. In some embodiments, the CRISPR gene-editing system for the treatment of gout comprises one or more sgRNAs targeting eTNFRSF4. In some embodiments, the CRISPR gene-editing system for the treatment of gout comprises one or more sgRNAs targeting fTNFRSF4.

In some embodiments, the CRISPR gene-editing system for the treatment of gout comprises one or more sgRNAs targeting exon 1 of hTNFRSF4. In some embodiments, the CRISPR gene-editing system for the treatment of gout comprises one or more sgRNAs targeting exon 2 of hTNFRSF4. In some embodiments, the CRISPR gene-editing system for the treatment of gout comprises one or more sgRNAs targeting exon 3 of hTNFRSF4. In some embodiments, the CRISPR gene-editing system for the treatment of gout comprises one or more sgRNAs targeting exon 4 of hTNFRSF4. In some embodiments, the CRISPR gene-editing system for the treatment of gout comprises one or more sgRNAs targeting exon 5 of hTNFRSF4. In some embodiments, the CRISPR gene-editing system for the treatment of gout comprises one or more sgRNAs targeting exon 6 of hTNFRSF4.

In some embodiments, the CRISPR gene-editing system for the treatment of gout comprises one or more sgRNAs targeting exon 1 of cTNFRSF4. In some embodiments, the CRISPR gene-editing system for the treatment of gout comprises one or more sgRNAs targeting exon 2 of cTNFRSF4. In some embodiments, the CRISPR gene-editing system for the treatment of gout comprises one or more sgRNAs targeting exon 3 of cTNFRSF4. In some embodiments, the CRISPR gene-editing system for the treatment of gout comprises one or more sgRNAs targeting exon 4 of cTNFRSF4. In some embodiments, the CRISPR gene-editing system for the treatment of gout comprises one or more sgRNAs targeting exon 5 of cTNFRSF4. In some embodiments, the CRISPR gene-editing system for the treatment of gout comprises one or more sgRNAs targeting exon 6 of cTNFRSF4. In some embodiments, the CRISPR gene-editing system for the treatment of gout comprises one or more sgRNAs targeting exon 7 of cTNFRSF4.

In some embodiments, the CRISPR gene-editing system for the treatment of gout comprises one or more sgRNAs targeting exon 1 of eTNFRSF4. In some embodiments, the CRISPR gene-editing system for the treatment of gout comprises one or more sgRNAs targeting exon 2 of eTNFRSF4. In some embodiments, the CRISPR gene-editing system for the treatment of gout comprises one or more sgRNAs targeting exon 3 of eTNFRSF4. In some embodiments, the CRISPR gene-editing system for the treatment of gout comprises one or more sgRNAs targeting exon 4 of eTNFRSF4. In some embodiments, the CRISPR gene-editing system for the treatment of gout comprises one or more sgRNAs targeting exon 5 of eTNFRSF4. In some embodiments, the CRISPR gene-editing system for the treatment of gout comprises one or more sgRNAs targeting exon 6 of eTNFRSF4. In some embodiments, the CRISPR gene-editing system for the treatment of gout comprises one or more sgRNAs targeting exon 7 of eTNFRSF4.

In some embodiments, the CRISPR gene-editing system for the treatment of gout comprises one or more sgRNAs targeting exon 1 of fTNFRSF4. In some embodiments, the CRISPR gene-editing system for the treatment of gout comprises one or more sgRNAs targeting exon 2 of fTNFRSF4. In some embodiments, the CRISPR gene-editing system for the treatment of gout comprises one or more sgRNAs targeting exon 3 of fTNFRSF4. In some embodiments, the CRISPR gene-editing system for the treatment of gout comprises one or more sgRNAs targeting exon 4 of fTNFRSF4. In some embodiments, the CRISPR gene-editing system for the treatment of gout comprises one or more sgRNAs targeting exon 5 of fTNFRSF4. In some embodiments, the CRISPR gene-editing system for the treatment of gout comprises one or more sgRNAs targeting exon 6 of fTNFRSF4. In some embodiments, the CRISPR gene-editing system for the treatment of gout comprises one or more sgRNAs targeting exon 7 of fTNFRSF4.

In some embodiments, the CRISPR gene-editing system for the treatment of gout comprises one or more sgRNAs targeting TNFRSF11A. In some embodiments, the CRISPR gene-editing system for the treatment of gout comprises one or more sgRNAs targeting hTNFRSF11A. In some embodiments, the CRISPR gene-editing system for the treatment of gout comprises one or more sgRNAs targeting cTNFRSF11A. In some embodiments, the CRISPR gene-editing system for the treatment of gout comprises one or more sgRNAs targeting eTNFRSF11A. In some embodiments, the CRISPR gene-editing system for the treatment of gout comprises one or more sgRNAs targeting fTNFRSF11A.

In some embodiments, the CRISPR gene-editing system for the treatment of gout comprises one or more sgRNAs targeting exon 1 of hTNFRSF11A. In some embodiments, the CRISPR gene-editing system for the treatment of gout comprises one or more sgRNAs targeting exon 2 of hTNFRSF11A. In some embodiments, the CRISPR gene-editing system for the treatment of gout comprises one or more sgRNAs targeting exon 3 of hTNFRSF11A. In some embodiments, the CRISPR gene-editing system for the treatment of gout comprises one or more sgRNAs targeting exon 4 of hTNFRSF11A. In some embodiments, the CRISPR gene-editing system for the treatment of gout comprises one or more sgRNAs targeting exon 5 of hTNFRSF11A. In some embodiments, the CRISPR gene-editing system for the treatment of gout comprises one or more sgRNAs targeting exon 6 of hTNFRSF11A. In some embodiments, the CRISPR gene-editing system for the treatment of gout comprises one or more sgRNAs targeting exon 7 of hTNFRSF11A. In some embodiments, the CRISPR gene-editing system for the treatment of gout comprises one or more sgRNAs targeting exon 8 of hTNFRSF11A. In some embodiments, the CRISPR gene-editing system for the treatment of gout comprises one or more sgRNAs targeting exon 9 of hTNFRSF11A. In some embodiments, the CRISPR gene-editing system for the treatment of gout comprises one or more sgRNAs targeting exon 10 of hTNFRSF11A. In some embodiments, the CRISPR gene-editing system for the treatment of gout comprises one or more sgRNAs targeting exon 11 of hTNFRSF11A. In some embodiments, the CRISPR gene-editing system for the treatment of gout comprises one or more sgRNAs targeting exon 12 of hTNFRSF11A.

In some embodiments, the CRISPR gene-editing system for the treatment of gout comprises one or more sgRNAs targeting exon 1 of cTNFRSF11A. In some embodiments, the CRISPR gene-editing system for the treatment of gout comprises one or more sgRNAs targeting exon 2 of cTNFRSF11A. In some embodiments, the CRISPR gene-editing system for the treatment of gout comprises one or more sgRNAs targeting exon 3 of cTNFRSF11A. In some embodiments, the CRISPR gene-editing system for the treatment of gout comprises one or more sgRNAs targeting exon 4 of cTNFRSF11A. In some embodiments, the CRISPR gene-editing system for the treatment of gout comprises one or more sgRNAs targeting exon 5 of cTNFRSF11A. In some embodiments, the CRISPR gene-editing system for the treatment of gout comprises one or more sgRNAs targeting exon 6 of cTNFRSF11A. In some embodiments, the CRISPR gene-editing system for the treatment of gout comprises one or more sgRNAs targeting exon 7 of cTNFRSF11A. In some embodiments, the CRISPR gene-editing system for the treatment of gout comprises one or more sgRNAs targeting exon 8 of cTNFRSF11A. In some embodiments, the CRISPR gene-editing system for the treatment of gout comprises one or more sgRNAs targeting exon 9 of cTNFRSF11A. In some embodiments, the CRISPR gene-editing system for the treatment of gout comprises one or more sgRNAs targeting exon 10 of cTNFRSF11A. In some embodiments, the CRISPR gene-editing system for the treatment of gout comprises one or more sgRNAs targeting exon 11 of cTNFRSF11A.

In some embodiments, the CRISPR gene-editing system for the treatment of gout comprises one or more sgRNAs targeting exon 1 of eTNFRSF11A. In some embodiments, the CRISPR gene-editing system for the treatment of gout comprises one or more sgRNAs targeting exon 2 of eTNFRSF11A. In some embodiments, the CRISPR gene-editing system for the treatment of gout comprises one or more sgRNAs targeting exon 3 of eTNFRSF11A. In some embodiments, the CRISPR gene-editing system for the treatment of gout comprises one or more sgRNAs targeting exon 4 of eTNFRSF11A. In some embodiments, the CRISPR gene-editing system for the treatment of gout comprises one or more sgRNAs targeting exon 5 of eTNFRSF11A. In some embodiments, the CRISPR gene-editing system for the treatment of gout comprises one or more sgRNAs targeting exon 6 of eTNFRSF11A. In some embodiments, the CRISPR gene-editing system for the treatment of gout comprises one or more sgRNAs targeting exon 7 of eTNFRSF11A. In some embodiments, the CRISPR gene-editing system for the treatment of gout comprises one or more sgRNAs targeting exon 8 of eTNFRSF11A. In some embodiments, the CRISPR gene-editing system for the treatment of gout comprises one or more sgRNAs targeting exon 9 of eTNFRSF11A. In some embodiments, the CRISPR gene-editing system for the treatment of gout comprises one or more sgRNAs targeting exon 10 of eTNFRSF11A.

In some embodiments, the CRISPR gene-editing system for the treatment of gout comprises one or more sgRNAs targeting exon 1 of fTNFRSF11A. In some embodiments, the CRISPR gene-editing system for the treatment of gout comprises one or more sgRNAs targeting exon 2 of fTNFRSF11A. In some embodiments, the CRISPR gene-editing system for the treatment of gout comprises one or more sgRNAs targeting exon 3 of fTNFRSF11A. In some embodiments, the CRISPR gene-editing system for the treatment of gout comprises one or more sgRNAs targeting exon 4 of fTNFRSF11A. In some embodiments, the CRISPR gene-editing system for the treatment of gout comprises one or more sgRNAs targeting exon 5 of fTNFRSF11A. In some embodiments, the CRISPR gene-editing system for the treatment of gout comprises one or more sgRNAs targeting exon 6 of fTNFRSF11A. In some embodiments, the CRISPR gene-editing system for the treatment of gout comprises one or more sgRNAs targeting exon 7 of fTNFRSF11A. In some embodiments, the CRISPR gene-editing system for the treatment of gout comprises one or more sgRNAs targeting exon 8 of fTNFRSF11A. In some embodiments, the CRISPR gene-editing system for the treatment of gout comprises one or more sgRNAs targeting exon 9 of fTNFRSF11A. In some embodiments, the CRISPR gene-editing system for the treatment of gout comprises one or more sgRNAs targeting exon 10 of fTNFRSF11A. In some embodiments, the CRISPR gene-editing system for the treatment of gout comprises one or more sgRNAs targeting exon 11 of fTNFRSF11A.

### V. Back or Spine Conditions or Disorders

### A. Introduction

Back or spine conditions or disorders, including low back pain, cervical pain, sacral pain, thoracic pain. and pain or inflammation associated with discogenic disorders e.g., degenerative disc disease (DDD) or internal disc disruption (IDD), are a major cause of morbidity and disability worldwide for which few long-term options for amelioration currently exist. Andersson GB. Epidemiological features of chronic low-back pain. Lancet. 1999; 354:581-585. Presently available treatments include surgical or less invasive options that often fail to offer long-term palliation. Ju, et al. Global Spine Journal (2020): 2192568220963058. All vertebrate species are affected by back or spine conditions or disorders, including working animals, domestic pets, and their owners. All suffer from the associated discomfort, pain, and disability, depending on the degree of disease progression.

Back or spine conditions or disorders, such as low back pain, are complex diseases characterized by a multitude of inputs contributing to a progressive course of disability. Among these contributors are morphological irregularities (e.g., disc disruptions), inflammation, changes in the localized cellular environment (e.g., vascularization and/or innervation) and degenerative changes. Peng, Bao-Gan. World Journal of Orthopedics 4.2 (2013): 42. Each contributing factor is driven by differential expression of various gene products, including at least pro-inflammatory cytokines, growth factors, pain signaling molecules, and other effector biomolecules. There is a pressing need for new methods and compositions to treat this spectrum of disease and its associated disability.

The present disclosure provides compositions and methods for back or spine conditions or disorders. Particularly, said conditions are treated by reducing pro-inflammatory signaling mediated by inflammatory cytokines, such as, IL1α, IL1β, TNF-α, IL6, IL8, IL18, IL33, matrix metalloproteinases (MMPs), or TGFB1, or TGFB2, binding to their cognate receptor(s). In some embodiments, such conditions or disorders include disorders of the intervertebral discs (IVDs). In some embodiments, the condition or disorder is DDD. In some embodiments, the condition or disorder is IDD. In some embodiments, the condition or disorder is low back pain.

Among the advantages of the present disclosure over treatments currently available for mammals afflicted with back or spine conditions or disorders include the period of relief from symptoms. Upon local administration (e.g., intradiscal injection) and subsequent genetic editing of a cell (e.g., a chondrocyte, a tenocyte, an osteocyte, a monocyte, a macrophage or the cells of the nucleus pulposus or annulus fibrosus), pro-inflammatory signaling is silenced through the targeted gene for the life of that cell. By contrast, biologic treatments require periodic dosing, which may magnify the impact of any side effects, which can be severe. Among various genetic approaches, the present disclosure is also superior due to the resistance to leakiness built in by virtue of modifying a protein receptor, rather than ablating its expression altogether.

### B. Low Back Pain

In one aspect, the present disclosure encompasses treatments for low back pain. In some embodiments, the low back pain treatment comprises a therapeutically effective amount of a Clustered Regularly Interspaced Short Palindromic Repeats (CRISPR) gene-editing system, the system comprising: (i) a CRISPR Associated (Cas) protein; and (ii) at least one guide RNA targeting IL1R1. In some embodiments, the low back pain treatment comprises a CRISPR gene-editing system targeting hIL1R1. In some embodiments, the low back pain treatment comprises a CRISPR gene-editing system targeting cIL1R1. In some embodiments, the low back pain treatment comprises a CRISP gene-editing system R targeting eIL1R1. In some embodiments, the low back pain treatment comprises a CRISPR gene-editing system targeting fIL1R1.

In some embodiments, the CRISPR gene-editing system for the treatment of low back pain comprises one or more sgRNAs targeting exon 1 of hIL1R1. In some embodiments, the CRISPR gene-editing system for the treatment of low back pain comprises one or more sgRNAs targeting exon 2 of hIL1R1. In some embodiments, the CRISPR gene-editing system for the treatment of low back pain comprises one or more sgRNAs targeting exon 3 of hIL1R1. In some embodiments, the CRISPR gene-editing system for the treatment of low back pain comprises one or more sgRNAs targeting exon 4 of hIL1R1. In some embodiments, the CRISPR gene-editing system for the treatment of low back pain comprises one or more sgRNAs targeting exon 5 of hIL1R1. In some embodiments, the CRISPR gene-editing system for the treatment of low back pain comprises one or more sgRNAs targeting exon 6 of hIL1R1. In some embodiments, the CRISPR gene-editing system for the treatment of low back pain comprises one or more sgRNAs targeting exon 7 of hIL1R1. In some embodiments, the CRISPR gene-editing system for the treatment of low back pain comprises one or more sgRNAs targeting exon 8 of hIL1R1. In some embodiments, the CRISPR gene-editing system for the treatment of low back pain comprises one or more sgRNAs targeting exon 9 of hIL1R1. In some embodiments, the CRISPR gene-editing system for the treatment of low back pain comprises one or more sgRNAs targeting exon 10 of hIL1R1. In some embodiments, the CRISPR gene-editing system for the treatment of low back pain comprises one or more sgRNAs targeting exon 11 of hIL1R1. In some embodiments, the CRISPR gene-editing system for the treatment of low back pain comprises one or more sgRNAs targeting exon 12 of hIL1R1. In some embodiments, the CRISPR gene-editing system for the treatment of low back pain comprises one or more sgRNAs targeting exon 13 of hIL1R1. In some embodiments, the CRISPR gene-editing system for the treatment of low back pain comprises one or more sgRNAs targeting exon 14 of hIL1R1. In some embodiments, the CRISPR gene-editing system for the treatment of low back pain comprises one or more sgRNAs targeting exon 15 of hIL1R1. In some embodiments, the CRISPR gene-editing system for the treatment of low back pain comprises one or more sgRNAs targeting exon 16 of hIL1R1. In some embodiments, the CRISPR gene-editing system for the treatment of low back pain comprises one or more sgRNAs targeting exon 17 of hIL1R1. In some embodiments, the CRISPR gene-editing system for the treatment of low back pain comprises one or more sgRNAs targeting exon 18 of hIL1R1. In some embodiments, the CRISPR gene-editing system for the treatment of low back pain comprises one or more sgRNAs targeting exon 19 of hIL1R1. In some embodiments, the CRISPR gene-editing system for the treatment of low back pain comprises one or more sgRNAs targeting exon 20 of hIL1R1. In some embodiments, the CRISPR gene-editing system for the treatment of low back pain comprises one or more sgRNAs targeting exon 21 of hIL1R1.

In some embodiments, the CRISPR gene-editing system for the treatment of low back pain comprises one or more sgRNAs targeting exon 1 of cIL1R1. In some embodiments, the CRISPR gene-editing system for the treatment of low back pain comprises one or more sgRNAs targeting exon 2 of cIL1R1. In some embodiments, the CRISPR gene-editing system for the treatment of low back pain comprises one or more sgRNAs targeting exon 3 of cIL1R1. In some embodiments, the CRISPR gene-editing system for the treatment of low back pain comprises one or more sgRNAs targeting exon 4 of cIL1R1. In some embodiments, the CRISPR gene-editing system for the treatment of low back pain comprises one or more sgRNAs targeting exon 5 of cIL1R1. In some embodiments, the CRISPR gene-editing system for the treatment of low back pain comprises one or more sgRNAs targeting exon 6 of cIL1R1. In some embodiments, the CRISPR gene-editing system for the treatment of low back pain comprises one or more sgRNAs targeting exon 7 of cIL1R1. In some embodiments, the CRISPR gene-editing system for the treatment of low back pain comprises one or more sgRNAs targeting exon 8 of cIL1R1. In some embodiments, the CRISPR gene-editing system for the treatment of low back pain comprises one or more sgRNAs targeting exon 9 of cIL1R1. In some embodiments, the CRISPR gene-editing system for the treatment of low back pain comprises one or more sgRNAs targeting exon 10 of cIL1R1. In some embodiments, the CRISPR gene-editing system for the treatment of low back pain comprises one or more sgRNAs targeting exon 11 of cIL1R1. In some embodiments, the CRISPR gene-editing system for the treatment of low back pain comprises one or more sgRNAs targeting exon 12 of cIL1R1. In some embodiments, the CRISPR gene-editing system for the treatment of low back pain comprises one or more sgRNAs targeting exon 13 of cIL1R1. In some embodiments, the CRISPR gene-editing system for the treatment of low back pain comprises one or more sgRNAs targeting exon 14 of cIL1R1. In some embodiments, the CRISPR gene-editing system for the treatment of low back pain comprises one or more sgRNAs targeting exon 15 of cIL1R1. In some embodiments, the CRISPR gene-editing system for the treatment of low back pain comprises one or more sgRNAs targeting exon 16 of cIL1R1.

In some embodiments, the CRISPR gene-editing system for the treatment of low back pain comprises one or more sgRNAs targeting exon 1 of eIL1R1. In some embodiments, the CRISPR gene-editing system for the treatment of low back pain comprises one or more sgRNAs targeting exon 2 of eIL1R1. In some embodiments, the CRISPR gene-editing system for the treatment of low back pain comprises one or more sgRNAs targeting exon 3 of eIL1R1. In some embodiments, the CRISPR gene-editing system for the treatment of low back pain comprises one or more sgRNAs targeting exon 4 of eIL1R1. In some embodiments, the CRISPR gene-editing system for the treatment of low back pain comprises one or more sgRNAs targeting exon 5 of eIL1R1. In some embodiments, the CRISPR gene-editing system for the treatment of low back pain comprises one or more sgRNAs targeting exon 6 of eIL1R1. In some embodiments, the CRISPR gene-editing system for the treatment of low back pain comprises one or more sgRNAs targeting exon 7 of eIL1R1. In some embodiments, the CRISPR gene-editing system for the treatment of low back pain comprises one or more sgRNAs targeting exon 8 of eIL1R1. In some embodiments, the CRISPR gene-editing system for the treatment of low back pain comprises one or more sgRNAs targeting exon 9 of eIL1R1. In some embodiments, the CRISPR gene-editing system for the treatment of low back pain comprises one or more sgRNAs targeting exon 10 of eIL1R1. In some embodiments, the CRISPR gene-editing system for the treatment of low back pain comprises one or more sgRNAs targeting exon 11 of eIL1R1. In some embodiments, the CRISPR gene-editing system for the treatment of low back pain comprises one or more sgRNAs targeting exon 12 of eIL1R1. In some embodiments, the CRISPR gene-editing system for the treatment of low back pain comprises one or more sgRNAs targeting exon 13 of eIL1R1. In some embodiments, the CRISPR gene-editing system for the treatment of low back pain comprises one or more sgRNAs targeting exon 14 of eIL1R1. In some embodiments, the CRISPR gene-editing system for the treatment of low back pain comprises one or more sgRNAs targeting exon 15 of eIL1R1.

In some embodiments, the CRISPR gene-editing system for the treatment of low back pain comprises one or more sgRNAs targeting exon 1 of fIL1R1. In some embodiments, the CRISPR gene-editing system for the treatment of low back pain comprises one or more sgRNAs targeting exon 2 of fIL1R1. In some embodiments, the CRISPR gene-editing system for the treatment of low back pain comprises one or more sgRNAs targeting exon 3 of fIL1R1. In some embodiments, the CRISPR gene-editing system for the treatment of low back pain comprises one or more sgRNAs targeting exon 4 of fIL1R1. In some embodiments, the CRISPR gene-editing system for the treatment of low back pain comprises one or more sgRNAs targeting exon 5 of fIL1R1. In some embodiments, the CRISPR gene-editing system for the treatment of low back pain comprises one or more sgRNAs targeting exon 6 of fIL1R1. In some embodiments, the CRISPR gene-editing system for the treatment of low back pain comprises one or more sgRNAs targeting exon 7 of fIL1R1. In some embodiments, the CRISPR gene-editing system for the treatment of low back pain comprises one or more sgRNAs targeting exon 8 of fIL1R1. In some embodiments, the CRISPR gene-editing system for the treatment of low back pain comprises one or more sgRNAs targeting exon 9 of fIL1R1. In some embodiments, the CRISPR gene-editing system for the treatment of low back pain comprises one or more sgRNAs targeting exon 10 of fIL1R1. In some embodiments, the CRISPR gene-editing system for the treatment of low back pain comprises one or more sgRNAs targeting exon 11 of fIL1R1. In some embodiments, the CRISPR gene-editing system for the treatment of low back pain comprises one or more sgRNAs targeting exon 12 of fIL1R1. In some embodiments, the CRISPR gene-editing system for the treatment of low back pain comprises one or more sgRNAs targeting exon 13 of fIL1R1. In some embodiments, the CRISPR gene-editing system for the treatment of low back pain comprises one or more sgRNAs targeting exon 14 of fIL1R1. In some embodiments, the CRISPR gene-editing system for the treatment of low back pain comprises one or more sgRNAs targeting exon 15 of fIL1R1. In some embodiments, the CRISPR gene-editing system for the treatment of low back pain comprises one or more sgRNAs targeting exon 16 of fIL1R1. In some embodiments, the CRISPR gene-editing system for the treatment of low back pain comprises one or more sgRNAs targeting exon 17 of fIL1R1. In some embodiments, the CRISPR gene-editing system for the treatment of low back pain comprises one or more sgRNAs targeting exon 18 of fIL1R1.

In some embodiments, the CRISPR gene-editing system for the treatment of low back pain comprises one or more sgRNAs targeting IL1RAP. In some embodiments, the CRISPR gene-editing system for the treatment of low back pain comprises one or more sgRNAs targeting hIL1RAP. In some embodiments, the CRISPR gene-editing system for the treatment of low back pain comprises one or more sgRNAs targeting cIL1RAP. In some embodiments, the CRISPR gene-editing system for the treatment of low back pain comprises one or more sgRNAs targeting eIL1RAP. In some embodiments, the CRISPR gene-editing system for the treatment of low back pain comprises one or more sgRNAs targeting fIL1RAP.

In some embodiments, the CRISPR gene-editing system for the treatment of low back pain comprises one or more sgRNAs targeting exon 1 of hIL1RAP. In some embodiments, the CRISPR gene-editing system for the treatment of low back pain comprises one or more sgRNAs targeting exon 2 of hIL1RAP. In some embodiments, the CRISPR gene-editing system for the treatment of low back pain comprises one or more sgRNAs targeting exon 3 of hIL1RAP. In some embodiments, the CRISPR gene-editing system for the treatment of low back pain comprises one or more sgRNAs targeting exon 4 of hIL1RAP. In some embodiments, the CRISPR gene-editing system for the treatment of low back pain comprises one or more sgRNAs targeting exon 5 of hIL1RAP. In some embodiments, the CRISPR gene-editing system for the treatment of low back pain comprises one or more sgRNAs targeting exon 6 of hIL1RAP. In some embodiments, the CRISPR gene-editing system for the treatment of low back pain comprises one or more sgRNAs targeting exon 7 of hIL1RAP. In some embodiments, the CRISPR gene-editing system for the treatment of low back pain comprises one or more sgRNAs targeting exon 8 of hIL1RAP. In some embodiments, the CRISPR gene-editing system for the treatment of low back pain comprises one or more sgRNAs targeting exon 9 of hIL1RAP. In some embodiments, the CRISPR gene-editing system for the treatment of low back pain comprises one or more sgRNAs targeting exon 10 of hIL1RAP. In some embodiments, the CRISPR gene-editing system for the treatment of low back pain comprises one or more sgRNAs targeting exon 11 of hIL1RAP. In some embodiments, the CRISPR gene-editing system for the treatment of low back pain comprises one or more sgRNAs targeting exon 12 of hIL1RAP. In some embodiments, the CRISPR gene-editing system for the treatment of low back pain comprises one or more sgRNAs targeting exon 13 of hIL1RAP. In some embodiments, the CRISPR gene-editing system for the treatment of low back pain comprises one or more sgRNAs targeting exon 14 of hIL1RAP. In some embodiments, the CRISPR gene-editing system for the treatment of low back pain comprises one or more sgRNAs targeting exon 15 of hIL1RAP. In some embodiments, the CRISPR gene-editing system for the treatment of low back pain comprises one or more sgRNAs targeting exon 16 of hIL1RAP. In some embodiments, the CRISPR gene-editing system for the treatment of low back pain comprises one or more sgRNAs targeting exon 17 of hIL1RAP.

In some embodiments, the CRISPR gene-editing system for the treatment of low back pain comprises one or more sgRNAs targeting exon 1 of cIL1RAP. In some embodiments, the CRISPR gene-editing system for the treatment of low back pain comprises one or more sgRNAs targeting exon 2 of cIL1RAP. In some embodiments, the CRISPR gene-editing system for the treatment of low back pain comprises one or more sgRNAs targeting exon 3 of cIL1RAP. In some embodiments, the CRISPR gene-editing system for the treatment of low back pain comprises one or more sgRNAs targeting exon 4 of cIL1RAP. In some embodiments, the CRISPR gene-editing system for the treatment of low back pain comprises one or more sgRNAs targeting exon 5 of cIL1RAP. In some embodiments, the CRISPR gene-editing system for the treatment of low back pain comprises one or more sgRNAs targeting exon 6 of cIL1RAP. In some embodiments, the CRISPR gene-editing system for the treatment of low back pain comprises one or more sgRNAs targeting exon 7 of cIL1RAP. In some embodiments, the CRISPR gene-editing system for the treatment of low back pain comprises one or more sgRNAs targeting exon 8 of cIL1RAP. In some embodiments, the CRISPR gene-editing system for the treatment of low back pain comprises one or more sgRNAs targeting exon 9 of cIL1RAP. In some embodiments, the CRISPR gene-editing system for the treatment of low back pain comprises one or more sgRNAs targeting exon 10 of cIL1RAP. In some embodiments, the CRISPR gene-editing system for the treatment of low back pain comprises one or more sgRNAs targeting exon 11 of cIL1RAP. In some embodiments, the CRISPR gene-editing system for the treatment of low back pain comprises one or more sgRNAs targeting exon 12 of cIL1RAP. In some embodiments, the CRISPR gene-editing system for the treatment of low back pain comprises one or more sgRNAs targeting exon 13 of cIL1RAP. In some embodiments, the CRISPR gene-editing system for the treatment of low back pain comprises one or more sgRNAs targeting exon 14 of cIL1RAP. In some embodiments, the CRISPR gene-editing system for the treatment of low back pain comprises one or more sgRNAs targeting exon 15 of cIL1RAP. In some embodiments, the CRISPR gene-editing system for the treatment of low back pain comprises one or more sgRNAs targeting exon 16 of cIL1RAP.

In some embodiments, the CRISPR gene-editing system for the treatment of low back pain comprises one or more sgRNAs targeting exon 1 of eIL1RAP. In some embodiments, the CRISPR gene-editing system for the treatment of low back pain comprises one or more sgRNAs targeting exon 2 of eIL1RAP. In some embodiments, the CRISPR gene-editing system for the treatment of low back pain comprises one or more sgRNAs targeting exon 3 of eIL1RAP. In some embodiments, the CRISPR gene-editing system for the treatment of low back pain comprises one or more sgRNAs targeting exon 4 of eIL1RAP. In some embodiments, the CRISPR gene-editing system for the treatment of low back pain comprises one or more sgRNAs targeting exon 5 of eIL1RAP. In some embodiments, the CRISPR gene-editing system for the treatment of low back pain comprises one or more sgRNAs targeting exon 6 of eIL1RAP. In some embodiments, the CRISPR gene-editing system for the treatment of low back pain comprises one or more sgRNAs targeting exon 7 of eIL1RAP. In some embodiments, the CRISPR gene-editing system for the treatment of low back pain comprises one or more sgRNAs targeting exon 8 of eIL1RAP. In some embodiments, the CRISPR gene-editing system for the treatment of low back pain comprises one or more sgRNAs targeting exon 9 of eIL1RAP. In some embodiments, the CRISPR gene-editing system for the treatment of low back pain comprises one or more sgRNAs targeting exon 10 of eIL1RAP. In some embodiments, the CRISPR gene-editing system for the treatment of low back pain comprises one or more sgRNAs targeting exon 11 of eIL1RAP. In some embodiments, the CRISPR gene-editing system for the treatment of low back pain comprises one or more sgRNAs targeting exon 12 of eIL1RAP. In some embodiments, the CRISPR gene-editing system for the treatment of low back pain comprises one or more sgRNAs targeting exon 13 of eIL1RAP.

In some embodiments, the CRISPR gene-editing system for the treatment of low back pain comprises one or more sgRNAs targeting exon 1 of fIL1RAP. In some embodiments, the CRISPR gene-editing system for the treatment of low back pain comprises one or more sgRNAs targeting exon 2 of fIL1RAP. In some embodiments, the CRISPR gene-editing system for the treatment of low back pain comprises one or more sgRNAs targeting exon 3 of fIL1RAP. In some embodiments, the CRISPR gene-editing system for the treatment of low back pain comprises one or more sgRNAs targeting exon 4 of fIL1RAP. In some embodiments, the CRISPR gene-editing system for the treatment of low back pain comprises one or more sgRNAs targeting exon 5 of fIL1RAP. In some embodiments, the CRISPR gene-editing system for the treatment of low back pain comprises one or more sgRNAs targeting exon 6 of fIL1RAP. In some embodiments, the CRISPR gene-editing system for the treatment of low back pain comprises one or more sgRNAs targeting exon 7 of fIL1RAP. In some embodiments, the CRISPR gene-editing system for the treatment of low back pain comprises one or more sgRNAs targeting exon 8 of fIL1RAP. In some embodiments, the CRISPR gene-editing system for the treatment of low back pain comprises one or more sgRNAs targeting exon 9 of fIL1RAP. In some embodiments, the CRISPR gene-editing system for the treatment of low back pain comprises one or more sgRNAs targeting exon 10 of fIL1RAP. In some embodiments, the CRISPR gene-editing system for the treatment of low back pain comprises one or more sgRNAs targeting exon 11 of fIL1RAP. In some embodiments, the CRISPR gene-editing system for the treatment of low back pain comprises one or more sgRNAs targeting exon 12 of fIL1RAP. In some embodiments, the CRISPR gene-editing system for the treatment of low back pain comprises one or more sgRNAs targeting exon 13 of fIL1RAP. In some embodiments, the CRISPR gene-editing system for the treatment of low back pain comprises one or more sgRNAs targeting exon 14 of fIL1RAP.

In some embodiments, the CRISPR gene-editing system for the treatment of low back pain comprises one or more sgRNAs targeting TGFBR1. In some embodiments, the CRISPR gene-editing system for the treatment of low back pain comprises one or more sgRNAs targeting hTGFBR1. In some embodiments, the CRISPR gene-editing system for the treatment of low back pain comprises one or more sgRNAs targeting cTGFBR1. In some embodiments, the CRISPR gene-editing system for the treatment of low back pain comprises one or more sgRNAs targeting e TGFBR1. In some embodiments, the CRISPR gene-editing system for the treatment of low back pain comprises one or more sgRNAs targeting fTGFBR1.

In some embodiments, the CRISPR gene-editing system for the treatment of low back pain comprises one or more sgRNAs targeting exon 1 of hTGFBR1. In some embodiments, the CRISPR gene-editing system for the treatment of low back pain comprises one or more sgRNAs targeting exon 2 of hTGFBR1. In some embodiments, the CRISPR gene-editing system for the treatment of low back pain comprises one or more sgRNAs targeting exon 3 of hTGFBR1. In some embodiments, the CRISPR gene-editing system for the treatment of low back pain comprises one or more sgRNAs targeting exon 4 of hTGFBR1. In some embodiments, the CRISPR gene-editing system for the treatment of low back pain comprises one or more sgRNAs targeting exon 5 of hTGFBR1. In some embodiments, the CRISPR gene-editing system for the treatment of low back pain comprises one or more sgRNAs targeting exon 6 of hTGFBR1. In some embodiments, the CRISPR gene-editing system for the treatment of low back pain comprises one or more sgRNAs targeting exon 7 of hTGFBR1. In some embodiments, the CRISPR gene-editing system for the treatment of low back pain comprises one or more sgRNAs targeting exon 8 of hTGFBR1. In some embodiments, the CRISPR gene-editing system for the treatment of low back pain comprises one or more sgRNAs targeting exon 9 of hTGFBR1. In some embodiments, the CRISPR gene-editing system for the treatment of low back pain comprises one or more sgRNAs targeting exon 10 of hTGFBR1. In some embodiments, the CRISPR gene-editing system for the treatment of low back pain comprises one or more sgRNAs targeting exon 11 of hTGFBR1.

In some embodiments, the CRISPR gene-editing system for the treatment of low back pain comprises one or more sgRNAs targeting exon 1 of cTGFBR1. In some embodiments, the CRISPR gene-editing system for the treatment of low back pain comprises one or more sgRNAs targeting exon 2 of cTGFBR1. In some embodiments, the CRISPR gene-editing system for the treatment of low back pain comprises one or more sgRNAs targeting exon 3 of cTGFBR1. In some embodiments, the CRISPR gene-editing system for the treatment of low back pain comprises one or more sgRNAs targeting exon 4 of cTGFBR1. In some embodiments, the CRISPR gene-editing system for the treatment of low back pain comprises one or more sgRNAs targeting exon 5 of cTGFBR1. In some embodiments, the CRISPR gene-editing system for the treatment of low back pain comprises one or more sgRNAs targeting exon 6 of cTGFBR1. In some embodiments, the CRISPR gene-editing system for the treatment of low back pain comprises one or more sgRNAs targeting exon 7 of cTGFBR1. In some embodiments, the CRISPR gene-editing system for the treatment of low back pain comprises one or more sgRNAs targeting exon 8 of cTGFBR1. In some embodiments, the CRISPR gene-editing system for the treatment of low back pain comprises one or more sgRNAs targeting exon 9 of cTGFBR1.

In some embodiments, the CRISPR gene-editing system for the treatment of low back pain comprises one or more sgRNAs targeting exon 1 of eTGFBR1. In some embodiments, the CRISPR gene-editing system for the treatment of low back pain comprises one or more sgRNAs targeting exon 2 of eTGFBR1. In some embodiments, the CRISPR gene-editing system for the treatment of low back pain comprises one or more sgRNAs targeting exon 3 of eTGFBR1. In some embodiments, the CRISPR gene-editing system for the treatment of low back pain comprises one or more sgRNAs targeting exon 4 of eTGFBR1. In some embodiments, the CRISPR gene-editing system for the treatment of low back pain comprises one or more sgRNAs targeting exon 5 of eTGFBR1. In some embodiments, the CRISPR gene-editing system for the treatment of low back pain comprises one or more sgRNAs targeting exon 6 of eTGFBR1. In some embodiments, the CRISPR gene-editing system for the treatment of low back pain comprises one or more sgRNAs targeting exon 7 of eTGFBR1. In some embodiments, the CRISPR gene-editing system for the treatment of low back pain comprises one or more sgRNAs targeting exon 8 of eTGFBR1. In some embodiments, the CRISPR gene-editing system for the treatment of low back pain comprises one or more sgRNAs targeting exon 9 of eTGFBR1. In some embodiments, the CRISPR gene-editing system for the treatment of low back pain comprises one or more sgRNAs targeting exon 10 of eTGFBR1. In some embodiments, the CRISPR gene-editing system for the treatment of low back pain comprises one or more sgRNAs targeting exon 11 of eTGFBR1. In some embodiments, the CRISPR gene-editing system for the treatment of low back pain comprises one or more sgRNAs targeting exon 12 of eTGFBR1.

In some embodiments, the CRISPR gene-editing system for the treatment of low back pain comprises one or more sgRNAs targeting exon 1 of fTGFBR1. In some embodiments, the CRISPR gene-editing system for the treatment of low back pain comprises one or more sgRNAs targeting exon 2 of fTGFBR1. In some embodiments, the CRISPR gene-editing system for the treatment of low back pain comprises one or more sgRNAs targeting exon 3 of fTGFBR1. In some embodiments, the CRISPR gene-editing system for the treatment of low back pain comprises one or more sgRNAs targeting exon 4 of fTGFBR1. In some embodiments, the CRISPR gene-editing system for the treatment of low back pain comprises one or more sgRNAs targeting exon 5 of fTGFBR1. In some embodiments, the CRISPR gene-editing system for the treatment of low back pain comprises one or more sgRNAs targeting exon 6 of fTGFBR1. In some embodiments, the CRISPR gene-editing system for the treatment of low back pain comprises one or more sgRNAs targeting exon 7 of fTGFBR1. In some embodiments, the CRISPR gene-editing system for the treatment of low back pain comprises one or more sgRNAs targeting exon 8 of fTGFBR1. In some embodiments, the CRISPR gene-editing system for the treatment of low back pain comprises one or more sgRNAs targeting exon 9 of fTGFBR1. In some embodiments, the CRISPR gene-editing system for the treatment of low back pain comprises one or more sgRNAs targeting exon 10 of fTGFBR1. In some embodiments, the CRISPR gene-editing system for the treatment of low back pain comprises one or more sgRNAs targeting exon 11 of fTGFBR1.

In some embodiments, the CRISPR gene-editing system for the treatment of low back pain comprises one or more sgRNAs targeting TGFBR2. In some embodiments, the CRISPR gene-editing system for the treatment of low back pain comprises one or more sgRNAs targeting hTGFBR2. In some embodiments, the CRISPR gene-editing system for the treatment of low back pain comprises one or more sgRNAs targeting cTGFBR2. In some embodiments, the CRISPR gene-editing system for the treatment of low back pain comprises one or more sgRNAs targeting eTGFBR2. In some embodiments, the CRISPR gene-editing system for the treatment of low back pain comprises one or more sgRNAs targeting fTGFBR2.

In some embodiments, the CRISPR gene-editing system for the treatment of low back pain comprises one or more sgRNAs targeting exon 1 of hTGFBR2. In some embodiments, the CRISPR gene-editing system for the treatment of low back pain comprises one or more sgRNAs targeting exon 2 of hTGFBR2. In some embodiments, the CRISPR gene-editing system for the treatment of low back pain comprises one or more sgRNAs targeting exon 3 of hTGFBR2. In some embodiments, the CRISPR gene-editing system for the treatment of low back pain comprises one or more sgRNAs targeting exon 4 of hTGFBR2. In some embodiments, the CRISPR gene-editing system for the treatment of low back pain comprises one or more sgRNAs targeting exon 5 of hTGFBR2. In some embodiments, the CRISPR gene-editing system for the treatment of low back pain comprises one or more sgRNAs targeting exon 6 of hTGFBR2. In some embodiments, the CRISPR gene-editing system for the treatment of low back pain comprises one or more sgRNAs targeting exon 7 of hTGFBR2. In some embodiments, the CRISPR gene-editing system for the treatment of low back pain comprises one or more sgRNAs targeting exon 8 of hTGFBR2. In some embodiments, the CRISPR gene-editing system for the treatment of low back pain comprises one or more sgRNAs targeting exon 9 of hTGFBR2.

In some embodiments, the CRISPR gene-editing system for the treatment of low back pain comprises one or more sgRNAs targeting exon 1 of cTGFBR2. In some embodiments, the CRISPR gene-editing system for the treatment of low back pain comprises one or more sgRNAs targeting exon 2 of cTGFBR2. In some embodiments, the CRISPR gene-editing system for the treatment of low back pain comprises one or more sgRNAs targeting exon 3 of cTGFBR2. In some embodiments, the CRISPR gene-editing system for the treatment of low back pain comprises one or more sgRNAs targeting exon 4 of cTGFBR2. In some embodiments, the CRISPR gene-editing system for the treatment of low back pain comprises one or more sgRNAs targeting exon 5 of cTGFBR2. In some embodiments, the CRISPR gene-editing system for the treatment of low back pain comprises one or more sgRNAs targeting exon 6 of cTGFBR2. In some embodiments, the CRISPR gene-editing system for the treatment of low back pain comprises one or more sgRNAs targeting exon 7 of cTGFBR2. In some embodiments, the CRISPR gene-editing system for the treatment of low back pain comprises one or more sgRNAs targeting exon 8 of cTGFBR2.

In some embodiments, the CRISPR gene-editing system for the treatment of low back pain comprises one or more sgRNAs targeting exon 1 of eTGFBR2. In some embodiments, the CRISPR gene-editing system for the treatment of low back pain comprises one or more sgRNAs targeting exon 2 of eTGFBR2. In some embodiments, the CRISPR gene-editing system for the treatment of low back pain comprises one or more sgRNAs targeting exon 3 of eTGFBR2. In some embodiments, the CRISPR gene-editing system for the treatment of low back pain comprises one or more sgRNAs targeting exon 4 of eTGFBR2. In some embodiments, the CRISPR gene-editing system for the treatment of low back pain comprises one or more sgRNAs targeting exon 5 of eTGFBR2. In some embodiments, the CRISPR gene-editing system for the treatment of low back pain comprises one or more sgRNAs targeting exon 6 of eTGFBR2. In some embodiments, the CRISPR gene-editing system for the treatment of low back pain comprises one or more sgRNAs targeting exon 7 of eTGFBR2. In some embodiments, the CRISPR gene-editing system for the treatment of low back pain comprises one or more sgRNAs targeting exon 8 of eTGFBR2. In some embodiments, the CRISPR gene-editing system for the treatment of low back pain comprises one or more sgRNAs targeting exon 9 of eTGFBR2. In some embodiments, the CRISPR gene-editing system for the treatment of low back pain comprises one or more sgRNAs targeting exon 10 of eTGFBR2.

In some embodiments, the CRISPR gene-editing system for the treatment of low back pain comprises one or more sgRNAs targeting exon 1 of fTGFBR2. In some embodiments, the CRISPR gene-editing system for the treatment of low back pain comprises one or more sgRNAs targeting exon 2 of fTGFBR2. In some embodiments, the CRISPR gene-editing system for the treatment of low back pain comprises one or more sgRNAs targeting exon 3 of fTGFBR2. In some embodiments, the CRISPR gene-editing system for the treatment of low back pain comprises one or more sgRNAs targeting exon 4 of fTGFBR2. In some embodiments, the CRISPR gene-editing system for the treatment of low back pain comprises one or more sgRNAs targeting exon 5 of fTGFBR2. In some embodiments, the CRISPR gene-editing system for the treatment of low back pain comprises one or more sgRNAs targeting exon 6 of fTGFBR2. In some embodiments, the CRISPR gene-editing system for the treatment of low back pain comprises one or more sgRNAs targeting exon 7 of fTGFBR2. In some embodiments, the CRISPR gene-editing system for the treatment of low back pain comprises one or more sgRNAs targeting exon 8 of fTGFBR2.

In some embodiments, the CRISPR gene-editing system for the treatment of low back pain comprises one or more sgRNAs targeting IL6R. In some embodiments, the CRISPR gene-editing system for the treatment of low back pain comprises one or more sgRNAs targeting hIL6R. In some embodiments, the CRISPR gene-editing system for the treatment of low back pain comprises one or more sgRNAs targeting cIL6R. In some embodiments, the CRISPR gene-editing system for the treatment of low back pain comprises one or more sgRNAs targeting eIL6R. In some embodiments, the CRISPR gene-editing system for the treatment of low back pain comprises one or more sgRNAs targeting fIL6R.

In some embodiments, the CRISPR gene-editing system for the treatment of low back pain comprises one or more sgRNAs targeting exon 1 of hIL6R. In some embodiments, the CRISPR gene-editing system for the treatment of low back pain comprises one or more sgRNAs targeting exon 2 of hIL6R. In some embodiments, the CRISPR gene-editing system for the treatment of low back pain comprises one or more sgRNAs targeting exon 3 of hIL6R. In some embodiments, the CRISPR gene-editing system for the treatment of low back pain comprises one or more sgRNAs targeting exon 4 of hIL6R. In some embodiments, the CRISPR gene-editing system for the treatment of low back pain comprises one or more sgRNAs targeting exon 5 of hIL6R. In some embodiments, the CRISPR gene-editing system for the treatment of low back pain comprises one or more sgRNAs targeting exon 6 of hIL6R. In some embodiments, the CRISPR gene-editing system for the treatment of low back pain comprises one or more sgRNAs targeting exon 7 of hIL6R. In some embodiments, the CRISPR gene-editing system for the treatment of low back pain comprises one or more sgRNAs targeting exon 8 of hIL6R. In some embodiments, the CRISPR gene-editing system for the treatment of low back pain comprises one or more sgRNAs targeting exon 9 of hIL6R. In some embodiments, the CRISPR gene-editing system for the treatment of low back pain comprises one or more sgRNAs targeting exon 10 of hIL6R. In some embodiments, the CRISPR gene-editing system for the treatment of low back pain comprises one or more sgRNAs targeting exon 11 of hIL6R In some embodiments, the CRISPR gene-editing system for the treatment of low back pain comprises one or more sgRNAs targeting exon 12 of hIL6R. In some embodiments, the CRISPR gene-editing system for the treatment of low back pain comprises one or more sgRNAs targeting exon 13 of hIL6R. In some embodiments, the CRISPR gene-editing system for the treatment of low back pain comprises one or more sgRNAs targeting exon 14 of hIL6R. In some embodiments, the CRISPR gene-editing system for the treatment of low back pain comprises one or more sgRNAs targeting exon 15 of hIL6R.

In some embodiments, the CRISPR gene-editing system for the treatment of low back pain comprises one or more sgRNAs targeting exon 1 of cIL6R. In some embodiments, the CRISPR gene-editing system for the treatment of low back pain comprises one or more sgRNAs targeting exon 2 of cIL6R. In some embodiments, the CRISPR gene-editing system for the treatment of low back pain comprises one or more sgRNAs targeting exon 3 of cIL6R. In some embodiments, the CRISPR gene-editing system for the treatment of low back pain comprises one or more sgRNAs targeting exon 4 of cIL6R. In some embodiments, the CRISPR gene-editing system for the treatment of low back pain comprises one or more sgRNAs targeting exon 5 of cIL6R. In some embodiments, the CRISPR gene-editing system for the treatment of low back pain comprises one or more sgRNAs targeting exon 6 of cIL6R. In some embodiments, the CRISPR gene-editing system for the treatment of low back pain comprises one or more sgRNAs targeting exon 7 of cIL6R. In some embodiments, the CRISPR gene-editing system for the treatment of low back pain comprises one or more sgRNAs targeting exon 8 of cIL6R. In some embodiments, the CRISPR gene-editing system for the treatment of low back pain comprises one or more sgRNAs targeting exon 9 of cIL6R. In some embodiments, the CRISPR gene-editing system for the treatment of low back pain comprises one or more sgRNAs targeting exon 10 of cIL6R. In some embodiments, the CRISPR gene-editing system for the treatment of low back pain comprises one or more sgRNAs targeting exon 11 of cIL6R. In some embodiments, the CRISPR gene-editing system for the treatment of low back pain comprises one or more sgRNAs targeting exon 12 of cIL6R. In some embodiments, the CRISPR gene-editing system for the treatment of low back pain comprises one or more sgRNAs targeting exon 13 of cIL6R.

In some embodiments, the CRISPR gene-editing system for the treatment of low back pain comprises one or more sgRNAs targeting exon 1 of eIL6R. In some embodiments, the CRISPR gene-editing system for the treatment of low back pain comprises one or more sgRNAs targeting exon 2 of eIL6R. In some embodiments, the CRISPR gene-editing system for the treatment of low back pain comprises one or more sgRNAs targeting exon 3 of eIL6R. In some embodiments, the CRISPR gene-editing system for the treatment of low back pain comprises one or more sgRNAs targeting exon 4 of eIL6R. In some embodiments, the CRISPR gene-editing system for the treatment of low back pain comprises one or more sgRNAs targeting exon 5 of eIL6R. In some embodiments, the CRISPR gene-editing system for the treatment of low back pain comprises one or more sgRNAs targeting exon 6 of eIL6R. In some embodiments, the CRISPR gene-editing system for the treatment of low back pain comprises one or more sgRNAs targeting exon 7 of eIL6R. In some embodiments, the CRISPR gene-editing system for the treatment of low back pain comprises one or more sgRNAs targeting exon 8 of eIL6R. In some embodiments, the CRISPR gene-editing system for the treatment of low back pain comprises one or more sgRNAs targeting exon 9 of eIL6R. In some embodiments, the CRISPR gene-editing system for the treatment of low back pain comprises one or more sgRNAs targeting exon 10 of eIL6R. In some embodiments, the CRISPR gene-editing system for the treatment of low back pain comprises one or more sgRNAs targeting exon 11 of eIL6R.

In some embodiments, the CRISPR gene-editing system for the treatment of low back pain comprises one or more sgRNAs targeting exon 1 of fIL6R. In some embodiments, the CRISPR gene-editing system for the treatment of low back pain comprises one or more sgRNAs targeting exon 2 of fIL6R. In some embodiments, the CRISPR gene-editing system for the treatment of low back pain comprises one or more sgRNAs targeting exon 3 of fIL6R. In some embodiments, the CRISPR gene-editing system for the treatment of low back pain comprises one or more sgRNAs targeting exon 4 of fIL6R. In some embodiments, the CRISPR gene-editing system for the treatment of low back pain comprises one or more sgRNAs targeting exon 5 of fIL6R. In some embodiments, the CRISPR gene-editing system for the treatment of low back pain comprises one or more sgRNAs targeting exon 6 of fIL6R. In some embodiments, the CRISPR gene-editing system for the treatment of low back pain comprises one or more sgRNAs targeting exon 7 of fIL6R. In some embodiments, the CRISPR gene-editing system for the treatment of low back pain comprises one or more sgRNAs targeting exon 8 of fIL6R. In some embodiments, the CRISPR gene-editing system for the treatment of low back pain comprises one or more sgRNAs targeting exon 9 of fIL6R. In some embodiments, the CRISPR gene-editing system for the treatment of low back pain comprises one or more sgRNAs targeting exon 10 of fIL6R.

In some embodiments, the CRISPR gene-editing system for the treatment of low back pain comprises one or more sgRNAs targeting IL6ST. In some embodiments, the CRISPR gene-editing system for the treatment of low back pain comprises one or more sgRNAs targeting hIL6ST. In some embodiments, the CRISPR gene-editing system for the treatment of low back pain comprises one or more sgRNAs targeting cIL6ST. In some embodiments, the CRISPR gene-editing system for the treatment of low back pain comprises one or more sgRNAs targeting eIL6ST. In some embodiments, the CRISPR gene-editing system for the treatment of low back pain comprises one or more sgRNAs targeting fIL6ST.

In some embodiments, the CRISPR gene-editing system for the treatment of low back pain comprises one or more sgRNAs targeting exon 1 of hIL6ST. In some embodiments, the CRISPR gene-editing system for the treatment of low back pain comprises one or more sgRNAs targeting exon 2 of hIL6ST. In some embodiments, the CRISPR gene-editing system for the treatment of low back pain comprises one or more sgRNAs targeting exon 3 of hIL6ST. In some embodiments, the CRISPR gene-editing system for the treatment of low back pain comprises one or more sgRNAs targeting exon 4 of hIL6ST. In some embodiments, the CRISPR gene-editing system for the treatment of low back pain comprises one or more sgRNAs targeting exon 5 of hIL6ST. In some embodiments, the CRISPR gene-editing system for the treatment of low back pain comprises one or more sgRNAs targeting exon 6 of hIL6ST. In some embodiments, the CRISPR gene-editing system for the treatment of low back pain comprises one or more sgRNAs targeting exon 7 of hIL6ST. In some embodiments, the CRISPR gene-editing system for the treatment of low back pain comprises one or more sgRNAs targeting exon 8 of hIL6ST. In some embodiments, the CRISPR gene-editing system for the treatment of low back pain comprises one or more sgRNAs targeting exon 9 of hIL6ST. In some embodiments, the CRISPR gene-editing system for the treatment of low back pain comprises one or more sgRNAs targeting exon 10 of hIL6ST. In some embodiments, the CRISPR gene-editing system for the treatment of low back pain comprises one or more sgRNAs targeting exon 11 of hIL6ST. In some embodiments, the CRISPR gene-editing system for the treatment of low back pain comprises one or more sgRNAs targeting exon 12 of hIL6ST. In some embodiments, the CRISPR gene-editing system for the treatment of low back pain comprises one or more sgRNAs targeting exon 13 of hIL6ST. In some embodiments, the CRISPR gene-editing system for the treatment of low back pain comprises one or more sgRNAs targeting exon 14 of hIL6ST. In some embodiments, the CRISPR gene-editing system for the treatment of low back pain comprises one or more sgRNAs targeting exon 15 of hIL6ST. In some embodiments, the CRISPR gene-editing system for the treatment of low back pain comprises one or more sgRNAs targeting exon 16 of hIL6ST. In some embodiments, the CRISPR gene-editing system for the treatment of low back pain comprises one or more sgRNAs targeting exon 17 of hIL6ST. In some embodiments, the CRISPR gene-editing system for the treatment of low back pain comprises one or more sgRNAs targeting exon 18 of hIL6ST. In some embodiments, the CRISPR gene-editing system for the treatment of low back pain comprises one or more sgRNAs targeting exon 19 of hIL6ST.

In some embodiments, the CRISPR gene-editing system for the treatment of low back pain comprises one or more sgRNAs targeting exon 1 of cIL6ST. In some embodiments, the CRISPR gene-editing system for the treatment of low back pain comprises one or more sgRNAs targeting exon 2 of cIL6ST. In some embodiments, the CRISPR gene-editing system for the treatment of low back pain comprises one or more sgRNAs targeting exon 3 of cIL6ST. In some embodiments, the CRISPR gene-editing system for the treatment of low back pain comprises one or more sgRNAs targeting exon 4 of cIL6ST. In some embodiments, the CRISPR gene-editing system for the treatment of low back pain comprises one or more sgRNAs targeting exon 5 of cIL6ST. In some embodiments, the CRISPR gene-editing system for the treatment of low back pain comprises one or more sgRNAs targeting exon 6 of cIL6ST. In some embodiments, the CRISPR gene-editing system for the treatment of low back pain comprises one or more sgRNAs targeting exon 7 of cIL6ST. In some embodiments, the CRISPR gene-editing system for the treatment of low back pain comprises one or more sgRNAs targeting exon 8 of cIL6ST. In some embodiments, the CRISPR gene-editing system for the treatment of low back pain comprises one or more sgRNAs targeting exon 9 of cIL6ST. In some embodiments, the CRISPR gene-editing system for the treatment of low back pain comprises one or more sgRNAs targeting exon 10 of cIL6ST. In some embodiments, the CRISPR gene-editing system for the treatment of low back pain comprises one or more sgRNAs targeting exon 11 of cIL6ST In some embodiments, the CRISPR gene-editing system for the treatment of low back pain comprises one or more sgRNAs targeting exon 12 of cIL6ST. In some embodiments, the CRISPR gene-editing system for the treatment of low back pain comprises one or more sgRNAs targeting exon 13 of cIL6ST. In some embodiments, the CRISPR gene-editing system for the treatment of low back pain comprises one or more sgRNAs targeting exon 14 of cIL6ST. In some embodiments, the CRISPR gene-editing system for the treatment of low back pain comprises one or more sgRNAs targeting exon 15 of cIL6ST. In some embodiments, the CRISPR gene-editing system for the treatment of low back pain comprises one or more sgRNAs targeting exon 16 of cIL6ST. In some embodiments, the CRISPR gene-editing system for the treatment of low back pain comprises one or more sgRNAs targeting exon 17 of cIL6ST. In some embodiments, the CRISPR gene-editing system for the treatment of low back pain comprises one or more sgRNAs targeting exon 18 of cIL6ST.

In some embodiments, the CRISPR gene-editing system for the treatment of low back pain comprises one or more sgRNAs targeting exon 1 of eIL6ST. In some embodiments, the CRISPR gene-editing system for the treatment of low back pain comprises one or more sgRNAs targeting exon 2 of eIL6ST. In some embodiments, the CRISPR gene-editing system for the treatment of low back pain comprises one or more sgRNAs targeting exon 3 of eIL6ST. In some embodiments, the CRISPR gene-editing system for the treatment of low back pain comprises one or more sgRNAs targeting exon 4 of eIL6ST. In some embodiments, the CRISPR gene-editing system for the treatment of low back pain comprises one or more sgRNAs targeting exon 5 of eIL6ST. In some embodiments, the CRISPR gene-editing system for the treatment of low back pain comprises one or more sgRNAs targeting exon 6 of eIL6ST. In some embodiments, the CRISPR gene-editing system for the treatment of low back pain comprises one or more sgRNAs targeting exon 7 of eIL6ST. In some embodiments, the CRISPR gene-editing system for the treatment of low back pain comprises one or more sgRNAs targeting exon 8 of eIL6ST. In some embodiments, the CRISPR gene-editing system for the treatment of low back pain comprises one or more sgRNAs targeting exon 9 of eIL6ST. In some embodiments, the CRISPR gene-editing system for the treatment of low back pain comprises one or more sgRNAs targeting exon 10 of eIL6ST. In some embodiments, the CRISPR gene-editing system for the treatment of low back pain comprises one or more sgRNAs targeting exon 11 of eIL6ST In some embodiments, the CRISPR gene-editing system for the treatment of low back pain comprises one or more sgRNAs targeting exon 12 of eIL6ST. In some embodiments, the CRISPR gene-editing system for the treatment of low back pain comprises one or more sgRNAs targeting exon 13 of eIL6ST. In some embodiments, the CRISPR gene-editing system for the treatment of low back pain comprises one or more sgRNAs targeting exon 14 of eIL6ST. In some embodiments, the CRISPR gene-editing system for the treatment of low back pain comprises one or more sgRNAs targeting exon 15 of eIL6ST. In some embodiments, the CRISPR gene-editing system for the treatment of low back pain comprises one or more sgRNAs targeting exon 16 of eIL6ST. In some embodiments, the CRISPR gene-editing system for the treatment of low back pain comprises one or more sgRNAs targeting exon 17 of eIL6ST. In some embodiments, the CRISPR gene-editing system for the treatment of low back pain comprises one or more sgRNAs targeting exon 18 of eIL6ST.

In some embodiments, the CRISPR gene-editing system for the treatment of low back pain comprises one or more sgRNAs targeting exon 1 of fIL6ST. In some embodiments, the CRISPR gene-editing system for the treatment of low back pain comprises one or more sgRNAs targeting exon 2 of fIL6ST. In some embodiments, the CRISPR gene-editing system for the treatment of low back pain comprises one or more sgRNAs targeting exon 3 of fIL6ST. In some embodiments, the CRISPR gene-editing system for the treatment of low back pain comprises one or more sgRNAs targeting exon 4 of fIL6ST. In some embodiments, the CRISPR gene-editing system for the treatment of low back pain comprises one or more sgRNAs targeting exon 5 of fIL6ST. In some embodiments, the CRISPR gene-editing system for the treatment of low back pain comprises one or more sgRNAs targeting exon 6 of fIL6ST. In some embodiments, the CRISPR gene-editing system for the treatment of low back pain comprises one or more sgRNAs targeting exon 7 of fIL6ST. In some embodiments, the CRISPR gene-editing system for the treatment of low back pain comprises one or more sgRNAs targeting exon 8 of fIL6ST. In some embodiments, the CRISPR gene-editing system for the treatment of low back pain comprises one or more sgRNAs targeting exon 9 of fIL6ST. In some embodiments, the CRISPR gene-editing system for the treatment of low back pain comprises one or more sgRNAs targeting exon 10 of fIL6ST. In some embodiments, the CRISPR gene-editing system for the treatment of low back pain comprises one or more sgRNAs targeting exon 11 of fIL6ST In some embodiments, the CRISPR gene-editing system for the treatment of low back pain comprises one or more sgRNAs targeting exon 12 of fIL6ST. In some embodiments, the CRISPR gene-editing system for the treatment of low back pain comprises one or more sgRNAs targeting exon 13 of fIL6ST. In some embodiments, the CRISPR gene-editing system for the treatment of low back pain comprises one or more sgRNAs targeting exon 14 of fIL6ST. In some embodiments, the CRISPR gene-editing system for the treatment of low back pain comprises one or more sgRNAs targeting exon 15 of fIL6ST. In some embodiments, the CRISPR gene-editing system for the treatment of low back pain comprises one or more sgRNAs targeting exon 16 of fIL6ST. In some embodiments, the CRISPR gene-editing system for the treatment of low back pain comprises one or more sgRNAs targeting exon 17 of fIL6ST. In some embodiments, the CRISPR gene-editing system for the treatment of low back pain comprises one or more sgRNAs targeting exon 18 of fIL6ST. In some embodiments, the CRISPR gene-editing system for the treatment of low back pain comprises one or more sgRNAs targeting exon 19 of fIL6ST. In some embodiments, the CRISPR gene-editing system for the treatment of low back pain comprises one or more sgRNAs targeting exon 20 of fIL6ST.

In some embodiments, the CRISPR gene-editing system for the treatment of low back pain comprises one or more sgRNAs targeting TNFRSF1A. In some embodiments, the CRISPR gene-editing system for the treatment of low back pain comprises one or more sgRNAs targeting hTNFRSF1A. In some embodiments, the CRISPR gene-editing system for the treatment of low back pain comprises one or more sgRNAs targeting cTNFRSF1A. In some embodiments, the CRISPR gene-editing system for the treatment of low back pain comprises one or more sgRNAs targeting eTNFRSF1A. In some embodiments, the CRISPR gene-editing system for the treatment of low back pain comprises one or more sgRNAs targeting fTNFRSF1A.

In some embodiments, the CRISPR gene-editing system for the treatment of low back pain comprises one or more sgRNAs targeting exon 1 of hTNFRSF1A. In some embodiments, the CRISPR gene-editing system for the treatment of low back pain comprises one or more sgRNAs targeting exon 2 of hTNFRSF1A. In some embodiments, the CRISPR gene-editing system for the treatment of low back pain comprises one or more sgRNAs targeting exon 3 of hTNFRSF1A. In some embodiments, the CRISPR gene-editing system for the treatment of low back pain comprises one or more sgRNAs targeting exon 4 of hTNFRSF1A. In some embodiments, the CRISPR gene-editing system for the treatment of low back pain comprises one or more sgRNAs targeting exon 5 of hTNFRSF1A. In some embodiments, the CRISPR gene-editing system for the treatment of low back pain comprises one or more sgRNAs targeting exon 6 of hTNFRSF1A. In some embodiments, the CRISPR gene-editing system for the treatment of low back pain comprises one or more sgRNAs targeting exon 7 of hTNFRSF1A. In some embodiments, the CRISPR gene-editing system for the treatment of low back pain comprises one or more sgRNAs targeting exon 8 of hTNFRSF1A. In some embodiments, the CRISPR gene-editing system for the treatment of low back pain comprises one or more sgRNAs targeting exon 9 of hTNFRSF1A. In some embodiments, the CRISPR gene-editing system for the treatment of low back pain comprises one or more sgRNAs targeting exon 10 of hTNFRSF1A. In some embodiments, the CRISPR gene-editing system for the treatment of low back pain comprises one or more sgRNAs targeting exon 11 of hTNFRSF1A.

In some embodiments, the CRISPR gene-editing system for the treatment of low back pain comprises one or more sgRNAs targeting exon 1 of cTNFRSF1A. In some embodiments, the CRISPR gene-editing system for the treatment of low back pain comprises one or more sgRNAs targeting exon 2 of cTNFRSF1A. In some embodiments, the CRISPR gene-editing system for the treatment of low back pain comprises one or more sgRNAs targeting exon 3 of cTNFRSF1A. In some embodiments, the CRISPR gene-editing system for the treatment of low back pain comprises one or more sgRNAs targeting exon 4 of cTNFRSF1A. In some embodiments, the CRISPR gene-editing system for the treatment of low back pain comprises one or more sgRNAs targeting exon 5 of cTNFRSF1A. In some embodiments, the CRISPR gene-editing system for the treatment of low back pain comprises one or more sgRNAs targeting exon 6 of cTNFRSF1A. In some embodiments, the CRISPR gene-editing system for the treatment of low back pain comprises one or more sgRNAs targeting exon 7 of cTNFRSF1A. In some embodiments, the CRISPR gene-editing system for the treatment of low back pain comprises one or more sgRNAs targeting exon 8 of cTNFRSF1A. In some embodiments, the CRISPR gene-editing system for the treatment of low back pain comprises one or more sgRNAs targeting exon 9 of cTNFRSF1A. In some embodiments, the CRISPR gene-editing system for the treatment of low back pain comprises one or more sgRNAs targeting exon 10 of cTNFRSF1A. In some embodiments, the CRISPR gene-editing system for the treatment of low back pain comprises one or more sgRNAs targeting exon 11 of cTNFRSF1A.

In some embodiments, the CRISPR gene-editing system for the treatment of low back pain comprises one or more sgRNAs targeting exon 1 of eTNFRSF1A. In some embodiments, the CRISPR gene-editing system for the treatment of low back pain comprises one or more sgRNAs targeting exon 2 of eTNFRSF1A. In some embodiments, the CRISPR gene-editing system for the treatment of low back pain comprises one or more sgRNAs targeting exon 3 of eTNFRSF1A. In some embodiments, the CRISPR gene-editing system for the treatment of low back pain comprises one or more sgRNAs targeting exon 4 of eTNFRSF1A. In some embodiments, the CRISPR gene-editing system for the treatment of low back pain comprises one or more sgRNAs targeting exon 5 of eTNFRSF1A. In some embodiments, the CRISPR gene-editing system for the treatment of low back pain comprises one or more sgRNAs targeting exon 6 of eTNFRSF1A. In some embodiments, the CRISPR gene-editing system for the treatment of low back pain comprises one or more sgRNAs targeting exon 7 of eTNFRSF1A. In some embodiments, the CRISPR gene-editing system for the treatment of low back pain comprises one or more sgRNAs targeting exon 8 of eTNFRSF1A. In some embodiments, the CRISPR gene-editing system for the treatment of low back pain comprises one or more sgRNAs targeting exon 9 of eTNFRSF1A. In some embodiments, the CRISPR gene-editing system for the treatment of low back pain comprises one or more sgRNAs targeting exon 10 of eTNFRSF1A.

In some embodiments, the CRISPR gene-editing system for the treatment of low back pain comprises one or more sgRNAs targeting exon 1 of fTNFRSF1A. In some embodiments, the CRISPR gene-editing system for the treatment of low back pain comprises one or more sgRNAs targeting exon 2 of fINFRSF1A. In some embodiments, the CRISPR gene-editing system for the treatment of low back pain comprises one or more sgRNAs targeting exon 3 of fTNFRSF1A. In some embodiments, the CRISPR gene-editing system for the treatment of low back pain comprises one or more sgRNAs targeting exon 4 of fTNFRSF1A. In some embodiments, the CRISPR gene-editing system for the treatment of low back pain comprises one or more sgRNAs targeting exon 5 of fTNFRSF1A. In some embodiments, the CRISPR gene-editing system for the treatment of low back pain comprises one or more sgRNAs targeting exon 6 of fTNFRSF1A. In some embodiments, the CRISPR gene-editing system for the treatment of low back pain comprises one or more sgRNAs targeting exon 7 of fTNFRSF1A. In some embodiments, the CRISPR gene-editing system for the treatment of low back pain comprises one or more sgRNAs targeting exon 8 of fTNFRSF1A. In some embodiments, the CRISPR gene-editing system for the treatment of low back pain comprises one or more sgRNAs targeting exon 9 of fTNFRSF1A. In some embodiments, the CRISPR gene-editing system for the treatment of low back pain comprises one or more sgRNAs targeting exon 10 of fTNFRSF1A.

In some embodiments, the CRISPR gene-editing system for the treatment of low back pain comprises one or more sgRNAs targeting TNFRSF1B. In some embodiments, the CRISPR gene-editing system for the treatment of low back pain comprises one or more sgRNAs targeting hTNFRSF1B. In some embodiments, the CRISPR gene-editing system for the treatment of low back pain comprises one or more sgRNAs targeting cTNFRSF1B. In some embodiments, the CRISPR gene-editing system for the treatment of low back pain comprises one or more sgRNAs targeting eTNFRSF1B. In some embodiments, the CRISPR gene-editing system for the treatment of low back pain comprises one or more sgRNAs targeting fTNFRSF1B.

In some embodiments, the CRISPR gene-editing system for the treatment of low back pain comprises one or more sgRNAs targeting exon 1 of hTNFRSF1B. In some embodiments, the CRISPR gene-editing system for the treatment of low back pain comprises one or more sgRNAs targeting exon 2 of hTNFRSF1B. In some embodiments, the CRISPR gene-editing system for the treatment of low back pain comprises one or more sgRNAs targeting exon 3 of hTNFRSF1B. In some embodiments, the CRISPR gene-editing system for the treatment of low back pain comprises one or more sgRNAs targeting exon 4 of hTNFRSF1B. In some embodiments, the CRISPR gene-editing system for the treatment of low back pain comprises one or more sgRNAs targeting exon 5 of hTNFRSF1B. In some embodiments, the CRISPR gene-editing system for the treatment of low back pain comprises one or more sgRNAs targeting exon 6 of hTNFRSF1B. In some embodiments, the CRISPR gene-editing system for the treatment of low back pain comprises one or more sgRNAs targeting exon 7 of hTNFRSF1B. In some embodiments, the CRISPR gene-editing system for the treatment of low back pain comprises one or more sgRNAs targeting exon 8 of hTNFRSF1B. In some embodiments, the CRISPR gene-editing system for the treatment of low back pain comprises one or more sgRNAs targeting exon 9 of hTNFRSF1B. In some embodiments, the CRISPR gene-editing system for the treatment of low back pain comprises one or more sgRNAs targeting exon 10 of hTNFRSF1B. In some embodiments, the CRISPR gene-editing system for the treatment of low back pain comprises one or more sgRNAs targeting exon 11 of hTNFRSF1B In some embodiments, the CRISPR gene-editing system for the treatment of low back pain comprises one or more sgRNAs targeting exon 12 of hTNFRSF1B. In some embodiments, the CRISPR gene-editing system for the treatment of low back pain comprises one or more sgRNAs targeting exon 13 of hTNFRSF1B.

In some embodiments, the CRISPR gene-editing system for the treatment of low back pain comprises one or more sgRNAs targeting exon 1 of cTNFRSF1B. In some embodiments, the CRISPR gene-editing system for the treatment of low back pain comprises one or more sgRNAs targeting exon 2 of cTNFRSF1B. In some embodiments, the CRISPR gene-editing system for the treatment of low back pain comprises one or more sgRNAs targeting exon 3 of cTNFRSF1B. In some embodiments, the CRISPR gene-editing system for the treatment of low back pain comprises one or more sgRNAs targeting exon 4 of cTNFRSF1B. In some embodiments, the CRISPR gene-editing system for the treatment of low back pain comprises one or more sgRNAs targeting exon 5 of cTNFRSF1B. In some embodiments, the CRISPR gene-editing system for the treatment of low back pain comprises one or more sgRNAs targeting exon 6 of cTNFRSF1B. In some embodiments, the CRISPR gene-editing system for the treatment of low back pain comprises one or more sgRNAs targeting exon 7 of cTNFRSF1B. In some embodiments, the CRISPR gene-editing system for the treatment of low back pain comprises one or more sgRNAs targeting exon 8 of CTNFRSF1B. In some embodiments, the CRISPR gene-editing system for the treatment of low back pain comprises one or more sgRNAs targeting exon 9 of cTNFRSF1B. In some embodiments, the CRISPR gene-editing system for the treatment of low back pain comprises one or more sgRNAs targeting exon 10 of cTNFRSF1B. In some embodiments, the CRISPR gene-editing system for the treatment of low back pain comprises one or more sgRNAs targeting exon 11 of cTNFRSF1B.

In some embodiments, the CRISPR gene-editing system for the treatment of low back pain comprises one or more sgRNAs targeting exon 1 of eTNFRSF1B. In some embodiments, the CRISPR gene-editing system for the treatment of low back pain comprises one or more sgRNAs targeting exon 2 of eTNFRSF1B. In some embodiments, the CRISPR gene-editing system for the treatment of low back pain comprises one or more sgRNAs targeting exon 3 of eTNFRSF1B. In some embodiments, the CRISPR gene-editing system for the treatment of low back pain comprises one or more sgRNAs targeting exon 4 of eTNFRSF1B. In some embodiments, the CRISPR gene-editing system for the treatment of low back pain comprises one or more sgRNAs targeting exon 5 of eTNFRSF1B. In some embodiments, the CRISPR gene-editing system for the treatment of low back pain comprises one or more sgRNAs targeting exon 6 of eTNFRSF1B. In some embodiments, the CRISPR gene-editing system for the treatment of low back pain comprises one or more sgRNAs targeting exon 7 of eTNFRSF1B. In some embodiments, the CRISPR gene-editing system for the treatment of low back pain comprises one or more sgRNAs targeting exon 8 of eTNFRSF1B. In some embodiments, the CRISPR gene-editing system for the treatment of low back pain comprises one or more sgRNAs targeting exon 9 of eTNFRSF1B. In some embodiments, the CRISPR gene-editing system for the treatment of low back pain comprises one or more sgRNAs targeting exon 10 of eTNFRSF1B. In some embodiments, the CRISPR gene-editing system for the treatment of low back pain comprises one or more sgRNAs targeting exon 11 of eTNFRSF1B.

In some embodiments, the CRISPR gene-editing system for the treatment of low back pain comprises one or more sgRNAs targeting exon 1 of fTNFRSF1B. In some embodiments, the CRISPR gene-editing system for the treatment of low back pain comprises one or more sgRNAs targeting exon 2 of fTNFRSF1B. In some embodiments, the CRISPR gene-editing system for the treatment of low back pain comprises one or more sgRNAs targeting exon 3 of fTNFRSF1B. In some embodiments, the CRISPR gene-editing system for the treatment of low back pain comprises one or more sgRNAs targeting exon 4 of fTNFRSF1B. In some embodiments, the CRISPR gene-editing system for the treatment of low back pain comprises one or more sgRNAs targeting exon 5 of fTNFRSF1B. In some embodiments, the CRISPR gene-editing system for the treatment of low back pain comprises one or more sgRNAs targeting exon 6 of fTNFRSF1B. In some embodiments, the CRISPR gene-editing system for the treatment of low back pain comprises one or more sgRNAs targeting exon 7 of fTNFRSF1B. In some embodiments, the CRISPR gene-editing system for the treatment of low back pain comprises one or more sgRNAs targeting exon 8 of fTNFRSF1B. In some embodiments, the CRISPR gene-editing system for the treatment of low back pain comprises one or more sgRNAs targeting exon 9 of fTNFRSF1B. In some embodiments, the CRISPR gene-editing system for the treatment of low back pain comprises one or more sgRNAs targeting exon 10 of fTNFRSF1B.

In some embodiments, the CRISPR gene-editing system for the treatment of low back pain comprises one or more sgRNAs targeting TNFRSF3. In some embodiments, the CRISPR gene-editing system for the treatment of low back pain comprises one or more sgRNAs targeting hTNFRSF3. In some embodiments, the CRISPR gene-editing system for the treatment of low back pain comprises one or more sgRNAs targeting cTNFRSF3. In some embodiments, the CRISPR gene-editing system for the treatment of low back pain comprises one or more sgRNAs targeting eTNFRSF3. In some embodiments, the CRISPR gene-editing system for the treatment of low back pain comprises one or more sgRNAs targeting fTNFRSF3.

In some embodiments, the CRISPR gene-editing system for the treatment of low back pain comprises one or more sgRNAs targeting exon 1 of hTNFRSF3. In some embodiments, the CRISPR gene-editing system for the treatment of low back pain comprises one or more sgRNAs targeting exon 2 of hTNFRSF3. In some embodiments, the CRISPR gene-editing system for the treatment of low back pain comprises one or more sgRNAs targeting exon 3 of hTNFRSF3. In some embodiments, the CRISPR gene-editing system for the treatment of low back pain comprises one or more sgRNAs targeting exon 4 of hTNFRSF3. In some embodiments, the CRISPR gene-editing system for the treatment of low back pain comprises one or more sgRNAs targeting exon 5 of hTNFRSF3. In some embodiments, the CRISPR gene-editing system for the treatment of low back pain comprises one or more sgRNAs targeting exon 6 of hTNFRSF3. In some embodiments, the CRISPR gene-editing system for the treatment of low back pain comprises one or more sgRNAs targeting exon 7 of hTNFRSF3. In some embodiments, the CRISPR gene-editing system for the treatment of low back pain comprises one or more sgRNAs targeting exon 8 of hTNFRSF3. In some embodiments, the CRISPR gene-editing system for the treatment of low back pain comprises one or more sgRNAs targeting exon 9 of hTNFRSF3. In some embodiments, the CRISPR gene-editing system for the treatment of low back pain comprises one or more sgRNAs targeting exon 10 of hTNFRSF3. In some embodiments, the CRISPR gene-editing system for the treatment of low back pain comprises one or more sgRNAs targeting exon 11 of hTNFRSF3. In some embodiments, the CRISPR gene-editing system for the treatment of low back pain comprises one or more sgRNAs targeting exon 12 of hTNFRSF3.

In some embodiments, the CRISPR gene-editing system for the treatment of low back pain comprises one or more sgRNAs targeting exon 1 of cTNFRSF3. In some embodiments, the CRISPR gene-editing system for the treatment of low back pain comprises one or more sgRNAs targeting exon 2 of cTNFRSF3. In some embodiments, the CRISPR gene-editing system for the treatment of low back pain comprises one or more sgRNAs targeting exon 3 of cTNFRSF3. In some embodiments, the CRISPR gene-editing system for the treatment of low back pain comprises one or more sgRNAs targeting exon 4 of cTNFRSF3. In some embodiments, the CRISPR gene-editing system for the treatment of low back pain comprises one or more sgRNAs targeting exon 5 of cTNFRSF3. In some embodiments, the CRISPR gene-editing system for the treatment of low back pain comprises one or more sgRNAs targeting exon 6 of cTNFRSF3. In some embodiments, the CRISPR gene-editing system for the treatment of low back pain comprises one or more sgRNAs targeting exon 7 of cTNFRSF3. In some embodiments, the CRISPR gene-editing system for the treatment of low back pain comprises one or more sgRNAs targeting exon 8 of cTNFRSF3. In some embodiments, the CRISPR gene-editing system for the treatment of low back pain comprises one or more sgRNAs targeting exon 9 of cTNFRSF3. In some embodiments, the CRISPR gene-editing system for the treatment of low back pain comprises one or more sgRNAs targeting exon 10 of cTNFRSF3.

In some embodiments, the CRISPR gene-editing system for the treatment of low back pain comprises one or more sgRNAs targeting exon 1 of eTNFRSF3. In some embodiments, the CRISPR gene-editing system for the treatment of low back pain comprises one or more sgRNAs targeting exon 2 of eTNFRSF3. In some embodiments, the CRISPR gene-editing system for the treatment of low back pain comprises one or more sgRNAs targeting exon 3 of eTNFRSF3. In some embodiments, the CRISPR gene-editing system for the treatment of low back pain comprises one or more sgRNAs targeting exon 4 of eTNFRSF3. In some embodiments, the CRISPR gene-editing system for the treatment of low back pain comprises one or more sgRNAs targeting exon 5 of eTNFRSF3. In some embodiments, the CRISPR gene-editing system for the treatment of low back pain comprises one or more sgRNAs targeting exon 6 of eTNFRSF3. In some embodiments, the CRISPR gene-editing system for the treatment of low back pain comprises one or more sgRNAs targeting exon 7 of eTNFRSF3. In some embodiments, the CRISPR gene-editing system for the treatment of low back pain comprises one or more sgRNAs targeting exon 8 of eTNFRSF3. In some embodiments, the CRISPR gene-editing system for the treatment of low back pain comprises one or more sgRNAs targeting exon 9 of eTNFRSF3. In some embodiments, the CRISPR gene-editing system for the treatment of low back pain comprises one or more sgRNAs targeting exon 10 of eTNFRSF3.

In some embodiments, the CRISPR gene-editing system for the treatment of low back pain comprises one or more sgRNAs targeting exon 1 of fTNFRSF3. In some embodiments, the CRISPR gene-editing system for the treatment of low back pain comprises one or more sgRNAs targeting exon 2 of fTNFRSF3. In some embodiments, the CRISPR gene-editing system for the treatment of low back pain comprises one or more sgRNAs targeting exon 3 of fTNFRSF3. In some embodiments, the CRISPR gene-editing system for the treatment of low back pain comprises one or more sgRNAs targeting exon 4 of fTNFRSF3. In some embodiments, the CRISPR gene-editing system for the treatment of low back pain comprises one or more sgRNAs targeting exon 5 of fTNFRSF3. In some embodiments, the CRISPR gene-editing system for the treatment of low back pain comprises one or more sgRNAs targeting exon 6 of fTNFRSF3. In some embodiments, the CRISPR gene-editing system for the treatment of low back pain comprises one or more sgRNAs targeting exon 7 of fTNFRSF3. In some embodiments, the CRISPR gene-editing system for the treatment of low back pain comprises one or more sgRNAs targeting exon 8 of fTNFRSF3. In some embodiments, the CRISPR gene-editing system for the treatment of low back pain comprises one or more sgRNAs targeting exon 9 of fTNFRSF3. In some embodiments, the CRISPR gene-editing system for the treatment of low back pain comprises one or more sgRNAs targeting exon 10 of fTNFRSF3.

In some embodiments, the CRISPR gene-editing system for the treatment of low back pain comprises one or more sgRNAs targeting TNFRSF4. In some embodiments, the CRISPR gene-editing system for the treatment of low back pain comprises one or more sgRNAs targeting hTNFRSF4. In some embodiments, the CRISPR gene-editing system for the treatment of low back pain comprises one or more sgRNAs targeting cTNFRSF4. In some embodiments, the CRISPR gene-editing system for the treatment of low back pain comprises one or more sgRNAs targeting eTNFRSF4. In some embodiments, the CRISPR gene-editing system for the treatment of low back pain comprises one or more sgRNAs targeting fTNFRSF4.

In some embodiments, the CRISPR gene-editing system for the treatment of low back pain comprises one or more sgRNAs targeting exon 1 of hTNFRSF4. In some embodiments, the CRISPR gene-editing system for the treatment of low back pain comprises one or more sgRNAs targeting exon 2 of hTNFRSF4. In some embodiments, the CRISPR gene-editing system for the treatment of low back pain comprises one or more sgRNAs targeting exon 3 of hTNFRSF4. In some embodiments, the CRISPR gene-editing system for the treatment of low back pain comprises one or more sgRNAs targeting exon 4 of hTNFRSF4. In some embodiments, the CRISPR gene-editing system for the treatment of low back pain comprises one or more sgRNAs targeting exon 5 of hTNFRSF4. In some embodiments, the CRISPR gene-editing system for the treatment of low back pain comprises one or more sgRNAs targeting exon 6 of hTNFRSF4.

In some embodiments, the CRISPR gene-editing system for the treatment of low back pain comprises one or more sgRNAs targeting exon 1 of cTNFRSF4. In some embodiments, the CRISPR gene-editing system for the treatment of low back pain comprises one or more sgRNAs targeting exon 2 of cTNFRSF4. In some embodiments, the CRISPR gene-editing system for the treatment of low back pain comprises one or more sgRNAs targeting exon 3 of cTNFRSF4. In some embodiments, the CRISPR gene-editing system for the treatment of low back pain comprises one or more sgRNAs targeting exon 4 of cTNFRSF4. In some embodiments, the CRISPR gene-editing system for the treatment of low back pain comprises one or more sgRNAs targeting exon 5 of cTNFRSF4. In some embodiments, the CRISPR gene-editing system for the treatment of low back pain comprises one or more sgRNAs targeting exon 6 of cTNFRSF4. In some embodiments, the CRISPR gene-editing system for the treatment of low back pain comprises one or more sgRNAs targeting exon 7 of cTNFRSF4.

In some embodiments, the CRISPR gene-editing system for the treatment of low back pain comprises one or more sgRNAs targeting exon 1 of eTNFRSF4. In some embodiments, the CRISPR gene-editing system for the treatment of low back pain comprises one or more sgRNAs targeting exon 2 of eTNFRSF4. In some embodiments, the CRISPR gene-editing system for the treatment of low back pain comprises one or more sgRNAs targeting exon 3 of eTNFRSF4. In some embodiments, the CRISPR gene-editing system for the treatment of low back pain comprises one or more sgRNAs targeting exon 4 of eTNFRSF4. In some embodiments, the CRISPR gene-editing system for the treatment of low back pain comprises one or more sgRNAs targeting exon 5 of eTNFRSF4. In some embodiments, the CRISPR gene-editing system for the treatment of low back pain comprises one or more sgRNAs targeting exon 6 of eTNFRSF4. In some embodiments, the CRISPR gene-editing system for the treatment of low back pain comprises one or more sgRNAs targeting exon 7 of eTNFRSF4.

In some embodiments, the CRISPR gene-editing system for the treatment of low back pain comprises one or more sgRNAs targeting exon 1 of fTNFRSF4. In some embodiments, the CRISPR gene-editing system for the treatment of low back pain comprises one or more sgRNAs targeting exon 2 of fTNFRSF4. In some embodiments, the CRISPR gene-editing system for the treatment of low back pain comprises one or more sgRNAs targeting exon 3 of fTNFRSF4. In some embodiments, the CRISPR gene-editing system for the treatment of low back pain comprises one or more sgRNAs targeting exon 4 of fTNFRSF4. In some embodiments, the CRISPR gene-editing system for the treatment of low back pain comprises one or more sgRNAs targeting exon 5 of fTNFRSF4. In some embodiments, the CRISPR gene-editing system for the treatment of low back pain comprises one or more sgRNAs targeting exon 6 of fTNFRSF4. In some embodiments, the CRISPR gene-editing system for the treatment of low back pain comprises one or more sgRNAs targeting exon 7 of fTNFRSF4.

In some embodiments, the CRISPR gene-editing system for the treatment of low back pain comprises one or more sgRNAs targeting TNFRSF11A. In some embodiments, the CRISPR gene-editing system for the treatment of low back pain comprises one or more sgRNAs targeting hTNFRSF11A. In some embodiments, the CRISPR gene-editing system for the treatment of low back pain comprises one or more sgRNAs targeting cTNFRSF11A. In some embodiments, the CRISPR gene-editing system for the treatment of low back pain comprises one or more sgRNAs targeting eTNFRSF11A. In some embodiments, the CRISPR gene-editing system for the treatment of low back pain comprises one or more sgRNAs targeting fTNFRSF11A.

In some embodiments, the CRISPR gene-editing system for the treatment of low back pain comprises one or more sgRNAs targeting exon 1 of hTNFRSF11A. In some embodiments, the CRISPR gene-editing system for the treatment of low back pain comprises one or more sgRNAs targeting exon 2 of hTNFRSF11A. In some embodiments, the CRISPR gene-editing system for the treatment of low back pain comprises one or more sgRNAs targeting exon 3 of hTNFRSF11A. In some embodiments, the CRISPR gene-editing system for the treatment of low back pain comprises one or more sgRNAs targeting exon 4 of hTNFRSF11A. In some embodiments, the CRISPR gene-editing system for the treatment of low back pain comprises one or more sgRNAs targeting exon 5 of hTNFRSF11A. In some embodiments, the CRISPR gene-editing system for the treatment of low back pain comprises one or more sgRNAs targeting exon 6 of hTNFRSF11A. In some embodiments, the CRISPR gene-editing system for the treatment of low back pain comprises one or more sgRNAs targeting exon 7 of hTNFRSF11A. In some embodiments, the CRISPR gene-editing system for the treatment of low back pain comprises one or more sgRNAs targeting exon 8 of hTNFRSF11A. In some embodiments, the CRISPR gene-editing system for the treatment of low back pain comprises one or more sgRNAs targeting exon 9 of hTNFRSF11A. In some embodiments, the CRISPR gene-editing system for the treatment of low back pain comprises one or more sgRNAs targeting exon 10 of hTNFRSF11A. In some embodiments, the CRISPR gene-editing system for the treatment of low back pain comprises one or more sgRNAs targeting exon 11 of hTNFRSF11A. In some embodiments, the CRISPR gene-editing system for the treatment of low back pain comprises one or more sgRNAs targeting exon 12 of hTNFRSF11A.

In some embodiments, the CRISPR gene-editing system for the treatment of low back pain comprises one or more sgRNAs targeting exon 1 of cTNFRSF11A. In some embodiments, the CRISPR gene-editing system for the treatment of low back pain comprises one or more sgRNAs targeting exon 2 of cTNFRSF11A. In some embodiments, the CRISPR gene-editing system for the treatment of low back pain comprises one or more sgRNAs targeting exon 3 of cTNFRSF11A. In some embodiments, the CRISPR gene-editing system for the treatment of low back pain comprises one or more sgRNAs targeting exon 4 of cTNFRSF11A. In some embodiments, the CRISPR gene-editing system for the treatment of low back pain comprises one or more sgRNAs targeting exon 5 of cTNFRSF11A. In some embodiments, the CRISPR gene-editing system for the treatment of low back pain comprises one or more sgRNAs targeting exon 6 of cTNFRSF11A. In some embodiments, the CRISPR gene-editing system for the treatment of low back pain comprises one or more sgRNAs targeting exon 7 of cTNFRSF11A. In some embodiments, the CRISPR gene-editing system for the treatment of low back pain comprises one or more sgRNAs targeting exon 8 of cTNFRSF11A. In some embodiments, the CRISPR gene-editing system for the treatment of low back pain comprises one or more sgRNAs targeting exon 9 of cTNFRSF11A. In some embodiments, the CRISPR gene-editing system for the treatment of low back pain comprises one or more sgRNAs targeting exon 10 of cTNFRSF11A. In some embodiments, the CRISPR gene-editing system for the treatment of low back pain comprises one or more sgRNAs targeting exon 11 of cTNFRSF11A.

In some embodiments, the CRISPR gene-editing system for the treatment of low back pain comprises one or more sgRNAs targeting exon 1 of eTNFRSF11A. In some embodiments, the CRISPR gene-editing system for the treatment of low back pain comprises one or more sgRNAs targeting exon 2 of eTNFRSF11A. In some embodiments, the CRISPR gene-editing system for the treatment of low back pain comprises one or more sgRNAs targeting exon 3 of eTNFRSF11A. In some embodiments, the CRISPR gene-editing system for the treatment of low back pain comprises one or more sgRNAs targeting exon 4 of eTNFRSF11A. In some embodiments, the CRISPR gene-editing system for the treatment of low back pain comprises one or more sgRNAs targeting exon 5 of eTNFRSF11A. In some embodiments, the CRISPR gene-editing system for the treatment of low back pain comprises one or more sgRNAs targeting exon 6 of eTNFRSF11A. In some embodiments, the CRISPR gene-editing system for the treatment of low back pain comprises one or more sgRNAs targeting exon 7 of eTNFRSF11A. In some embodiments, the CRISPR gene-editing system for the treatment of low back pain comprises one or more sgRNAs targeting exon 8 of eTNFRSF11A. In some embodiments, the CRISPR gene-editing system for the treatment of low back pain comprises one or more sgRNAs targeting exon 9 of eTNFRSF11A. In some embodiments, the CRISPR gene-editing system for the treatment of low back pain comprises one or more sgRNAs targeting exon 10 of eTNFRSF11A.

In some embodiments, the CRISPR gene-editing system for the treatment of low back pain comprises one or more sgRNAs targeting exon 1 of fTNFRSF11A. In some embodiments, the CRISPR gene-editing system for the treatment of low back pain comprises one or more sgRNAs targeting exon 2 of fTNFRSF11A. In some embodiments, the CRISPR gene-editing system for the treatment of low back pain comprises one or more sgRNAs targeting exon 3 of fTNFRSF11A. In some embodiments, the CRISPR gene-editing system for the treatment of low back pain comprises one or more sgRNAs targeting exon 4 of fTNFRSF11A. In some embodiments, the CRISPR gene-editing system for the treatment of low back pain comprises one or more sgRNAs targeting exon 5 of fTNFRSF11A. In some embodiments, the CRISPR gene-editing system for the treatment of low back pain comprises one or more sgRNAs targeting exon 6 of fTNFRSF11A. In some embodiments, the CRISPR gene-editing system for the treatment of low back pain comprises one or more sgRNAs targeting exon 7 of fTNFRSF11A. In some embodiments, the CRISPR gene-editing system for the treatment of low back pain comprises one or more sgRNAs targeting exon 8 of fTNFRSF11A. In some embodiments, the CRISPR gene-editing system for the treatment of low back pain comprises one or more sgRNAs targeting exon 9 of fTNFRSF11A. In some embodiments, the CRISPR gene-editing system for the treatment of low back pain comprises one or more sgRNAs targeting exon 10 of fTNFRSF11A. In some embodiments, the CRISPR gene-editing system for the treatment of low back pain comprises one or more sgRNAs targeting exon 11 of fTNFRSF11A.

### C. DDD

In one aspect, the present disclosure encompasses treatments for degenerative disc disorder (DDD). In some embodiments, the DDD treatment comprises a therapeutically effective amount of a Clustered Regularly Interspaced Short Palindromic Repeats (CRISPR) gene-editing system, the system comprising: (i) a CRISPR Associated (Cas) protein; and (ii) at least one guide RNA targeting IL1R1. In some embodiments, the DDD treatment comprises a CRISPR gene-editing system targeting hIL1R1. In some embodiments, the DDD treatment comprises a CRISPR gene-editing system targeting cIL1R1. In some embodiments, the DDD treatment comprises a CRISP gene-editing system R targeting eIL1R1. In some embodiments, the DDD treatment comprises a CRISPR gene-editing system targeting fIL1R1.

In some embodiments, the CRISPR gene-editing system for the treatment of DDD comprises one or more sgRNAs targeting exon 1 of hIL1R1. In some embodiments, the CRISPR gene-editing system for the treatment of DDD comprises one or more sgRNAs targeting exon 2 of hIL1R1. In some embodiments, the CRISPR gene-editing system for the treatment of DDD comprises one or more sgRNAs targeting exon 3 of hIL1R1. In some embodiments, the CRISPR gene-editing system for the treatment of DDD comprises one or more sgRNAs targeting exon 4 of hIL1R1. In some embodiments, the CRISPR gene-editing system for the treatment of DDD comprises one or more sgRNAs targeting exon 5 of hIL1R1. In some embodiments, the CRISPR gene-editing system for the treatment of DDD comprises one or more sgRNAs targeting exon 6 of hIL1R1. In some embodiments, the CRISPR gene-editing system for the treatment of DDD comprises one or more sgRNAs targeting exon 7 of hIL1R1. In some embodiments, the CRISPR gene-editing system for the treatment of DDD comprises one or more sgRNAs targeting exon 8 of hIL1R1. In some embodiments, the CRISPR gene-editing system for the treatment of DDD comprises one or more sgRNAs targeting exon 9 of hIL1R1. In some embodiments, the CRISPR gene-editing system for the treatment of DDD comprises one or more sgRNAs targeting exon 10 of hIL1R1. In some embodiments, the CRISPR gene-editing system for the treatment of DDD comprises one or more sgRNAs targeting exon 11 of hIL1R1. In some embodiments, the CRISPR gene-editing system for the treatment of DDD comprises one or more sgRNAs targeting exon 12 of hIL1R1. In some embodiments, the CRISPR gene-editing system for the treatment of DDD comprises one or more sgRNAs targeting exon 13 of hIL1R1. In some embodiments, the CRISPR gene-editing system for the treatment of DDD comprises one or more sgRNAs targeting exon 14 of hIL1R1. In some embodiments, the CRISPR gene-editing system for the treatment of DDD comprises one or more sgRNAs targeting exon 15 of hIL1R1. In some embodiments, the CRISPR gene-editing system for the treatment of DDD comprises one or more sgRNAs targeting exon 16 of hIL1R1. In some embodiments, the CRISPR gene-editing system for the treatment of DDD comprises one or more sgRNAs targeting exon 17 of hIL1R1. In some embodiments, the CRISPR gene-editing system for the treatment of DDD comprises one or more sgRNAs targeting exon 18 of hIL1R1. In some embodiments, the CRISPR gene-editing system for the treatment of DDD comprises one or more sgRNAs targeting exon 19 of hIL1R1. In some embodiments, the CRISPR gene-editing system for the treatment of DDD comprises one or more sgRNAs targeting exon 20 of hIL1R1. In some embodiments, the CRISPR gene-editing system for the treatment of DDD comprises one or more sgRNAs targeting exon 21 of hIL1R1.

In some embodiments, the CRISPR gene-editing system for the treatment of DDD comprises one or more sgRNAs targeting exon 1 of cIL1R1. In some embodiments, the CRISPR gene-editing system for the treatment of DDD comprises one or more sgRNAs targeting exon 2 of cIL1R1. In some embodiments, the CRISPR gene-editing system for the treatment of DDD comprises one or more sgRNAs targeting exon 3 of cIL1R1. In some embodiments, the CRISPR gene-editing system for the treatment of DDD comprises one or more sgRNAs targeting exon 4 of cIL1R1. In some embodiments, the CRISPR gene-editing system for the treatment of DDD comprises one or more sgRNAs targeting exon 5 of cIL1R1. In some embodiments, the CRISPR gene-editing system for the treatment of DDD comprises one or more sgRNAs targeting exon 6 of cIL1R1. In some embodiments, the CRISPR gene-editing system for the treatment of DDD comprises one or more sgRNAs targeting exon 7 of cIL1R1. In some embodiments, the CRISPR gene-editing system for the treatment of DDD comprises one or more sgRNAs targeting exon 8 of cIL1R1. In some embodiments, the CRISPR gene-editing system for the treatment of DDD comprises one or more sgRNAs targeting exon 9 of cIL1R1. In some embodiments, the CRISPR gene-editing system for the treatment of DDD comprises one or more sgRNAs targeting exon 10 of cIL1R1. In some embodiments, the CRISPR gene-editing system for the treatment of DDD comprises one or more sgRNAs targeting exon 11 of cIL1R1. In some embodiments, the CRISPR gene-editing system for the treatment of DDD comprises one or more sgRNAs targeting exon 12 of cIL1R1. In some embodiments, the CRISPR gene-editing system for the treatment of DDD comprises one or more sgRNAs targeting exon 13 of cIL1R1. In some embodiments, the CRISPR gene-editing system for the treatment of DDD comprises one or more sgRNAs targeting exon 14 of cIL1R1. In some embodiments, the CRISPR gene-editing system for the treatment of DDD comprises one or more sgRNAs targeting exon 15 of cIL1R1. In some embodiments, the CRISPR gene-editing system for the treatment of DDD comprises one or more sgRNAs targeting exon 16 of cIL1R1.

In some embodiments, the CRISPR gene-editing system for the treatment of DDD comprises one or more sgRNAs targeting exon 1 of eIL1R1. In some embodiments, the CRISPR gene-editing system for the treatment of DDD comprises one or more sgRNAs targeting exon 2 of eIL1R1. In some embodiments, the CRISPR gene-editing system for the treatment of DDD comprises one or more sgRNAs targeting exon 3 of eIL1R1. In some embodiments, the CRISPR gene-editing system for the treatment of DDD comprises one or more sgRNAs targeting exon 4 of eIL1R1. In some embodiments, the CRISPR gene-editing system for the treatment of DDD comprises one or more sgRNAs targeting exon 5 of eIL1R1. In some embodiments, the CRISPR gene-editing system for the treatment of DDD comprises one or more sgRNAs targeting exon 6 of eIL1R1. In some embodiments, the CRISPR gene-editing system for the treatment of DDD comprises one or more sgRNAs targeting exon 7 of eIL1R1. In some embodiments, the CRISPR gene-editing system for the treatment of DDD comprises one or more sgRNAs targeting exon 8 of eIL1R1. In some embodiments, the CRISPR gene-editing system for the treatment of DDD comprises one or more sgRNAs targeting exon 9 of eIL1R1. In some embodiments, the CRISPR gene-editing system for the treatment of DDD comprises one or more sgRNAs targeting exon 10 of eIL1R1. In some embodiments, the CRISPR gene-editing system for the treatment of DDD comprises one or more sgRNAs targeting exon 11 of eIL1R1. In some embodiments, the CRISPR gene-editing system for the treatment of DDD comprises one or more sgRNAs targeting exon 12 of eIL1R1. In some embodiments, the CRISPR gene-editing system for the treatment of DDD comprises one or more sgRNAs targeting exon 13 of eIL1R1. In some embodiments, the CRISPR gene-editing system for the treatment of DDD comprises one or more sgRNAs targeting exon 14 of eIL1R1. In some embodiments, the CRISPR gene-editing system for the treatment of DDD comprises one or more sgRNAs targeting exon 15 of eIL1R1.

In some embodiments, the CRISPR gene-editing system for the treatment of DDD comprises one or more sgRNAs targeting exon 1 of fIL1R1. In some embodiments, the CRISPR gene-editing system for the treatment of DDD comprises one or more sgRNAs targeting exon 2 of fIL1R1. In some embodiments, the CRISPR gene-editing system for the treatment of DDD comprises one or more sgRNAs targeting exon 3 of fIL1R1. In some embodiments, the CRISPR gene-editing system for the treatment of DDD comprises one or more sgRNAs targeting exon 4 of fIL1R1. In some embodiments, the CRISPR gene-editing system for the treatment of DDD comprises one or more sgRNAs targeting exon 5 of fIL1R1. In some embodiments, the CRISPR gene-editing system for the treatment of DDD comprises one or more sgRNAs targeting exon 6 of fIL1R1. In some embodiments, the CRISPR gene-editing system for the treatment of DDD comprises one or more sgRNAs targeting exon 7 of fIL1R1. In some embodiments, the CRISPR gene-editing system for the treatment of DDD comprises one or more sgRNAs targeting exon 8 of fIL1R1. In some embodiments, the CRISPR gene-editing system for the treatment of DDD comprises one or more sgRNAs targeting exon 9 of fIL1R1. In some embodiments, the CRISPR gene-editing system for the treatment of DDD comprises one or more sgRNAs targeting exon 10 of fIL1R1. In some embodiments, the CRISPR gene-editing system for the treatment of DDD comprises one or more sgRNAs targeting exon 11 of fIL1R1. In some embodiments, the CRISPR gene-editing system for the treatment of DDD comprises one or more sgRNAs targeting exon 12 of fIL1R1. In some embodiments, the CRISPR gene-editing system for the treatment of DDD comprises one or more sgRNAs targeting exon 13 of fIL1R1. In some embodiments, the CRISPR gene-editing system for the treatment of DDD comprises one or more sgRNAs targeting exon 14 of fIL1R1. In some embodiments, the CRISPR gene-editing system for the treatment of DDD comprises one or more sgRNAs targeting exon 15 of fIL1R1. In some embodiments, the CRISPR gene-editing system for the treatment of DDD comprises one or more sgRNAs targeting exon 16 of fIL1R1. In some embodiments, the CRISPR gene-editing system for the treatment of DDD comprises one or more sgRNAs targeting exon 17 of fIL1R1. In some embodiments, the CRISPR gene-editing system for the treatment of DDD comprises one or more sgRNAs targeting exon 18 of fIL1R1.

In some embodiments, the CRISPR gene-editing system for the treatment of DDD comprises one or more sgRNAs targeting IL1RAP. In some embodiments, the CRISPR gene-editing system for the treatment of DDD comprises one or more sgRNAs targeting hIL1RAP. In some embodiments, the CRISPR gene-editing system for the treatment of DDD comprises one or more sgRNAs targeting cIL1RAP. In some embodiments, the CRISPR gene-editing system for the treatment of DDD comprises one or more sgRNAs targeting eIL1RAP. In some embodiments, the CRISPR gene-editing system for the treatment of DDD comprises one or more sgRNAs targeting fIL1RAP.

In some embodiments, the CRISPR gene-editing system for the treatment of DDD comprises one or more sgRNAs targeting exon 1 of hIL1RAP. In some embodiments, the CRISPR gene-editing system for the treatment of DDD comprises one or more sgRNAs targeting exon 2 of hIL1RAP. In some embodiments, the CRISPR gene-editing system for the treatment of DDD comprises one or more sgRNAs targeting exon 3 of hIL1RAP. In some embodiments, the CRISPR gene-editing system for the treatment of DDD comprises one or more sgRNAs targeting exon 4 of hIL1RAP. In some embodiments, the CRISPR gene-editing system for the treatment of DDD comprises one or more sgRNAs targeting exon 5 of hIL1RAP. In some embodiments, the CRISPR gene-editing system for the treatment of DDD comprises one or more sgRNAs targeting exon 6 of hIL1RAP. In some embodiments, the CRISPR gene-editing system for the treatment of DDD comprises one or more sgRNAs targeting exon 7 of hIL1RAP. In some embodiments, the CRISPR gene-editing system for the treatment of DDD comprises one or more sgRNAs targeting exon 8 of hIL1RAP. In some embodiments, the CRISPR gene-editing system for the treatment of DDD comprises one or more sgRNAs targeting exon 9 of hIL1RAP. In some embodiments, the CRISPR gene-editing system for the treatment of DDD comprises one or more sgRNAs targeting exon 10 of hIL1RAP. In some embodiments, the CRISPR gene-editing system for the treatment of DDD comprises one or more sgRNAs targeting exon 11 of hIL1RAP. In some embodiments, the CRISPR gene-editing system for the treatment of DDD comprises one or more sgRNAs targeting exon 12 of hIL1RAP. In some embodiments, the CRISPR gene-editing system for the treatment of DDD comprises one or more sgRNAs targeting exon 13 of hIL1RAP. In some embodiments, the CRISPR gene-editing system for the treatment of DDD comprises one or more sgRNAs targeting exon 14 of hIL1RAP. In some embodiments, the CRISPR gene-editing system for the treatment of DDD comprises one or more sgRNAs targeting exon 15 of hIL1RAP. In some embodiments, the CRISPR gene-editing system for the treatment of DDD comprises one or more sgRNAs targeting exon 16 of hIL1RAP. In some embodiments, the CRISPR gene-editing system for the treatment of DDD comprises one or more sgRNAs targeting exon 17 of hIL1RAP.

In some embodiments, the CRISPR gene-editing system for the treatment of DDD comprises one or more sgRNAs targeting exon 1 of cIL1RAP. In some embodiments, the CRISPR gene-editing system for the treatment of DDD comprises one or more sgRNAs targeting exon 2 of cIL1RAP. In some embodiments, the CRISPR gene-editing system for the treatment of DDD comprises one or more sgRNAs targeting exon 3 of cIL1RAP. In some embodiments, the CRISPR gene-editing system for the treatment of DDD comprises one or more sgRNAs targeting exon 4 of cIL1RAP. In some embodiments, the CRISPR gene-editing system for the treatment of DDD comprises one or more sgRNAs targeting exon 5 of cIL1RAP. In some embodiments, the CRISPR gene-editing system for the treatment of DDD comprises one or more sgRNAs targeting exon 6 of cIL1RAP. In some embodiments, the CRISPR gene-editing system for the treatment of DDD comprises one or more sgRNAs targeting exon 7 of cIL1RAP. In some embodiments, the CRISPR gene-editing system for the treatment of DDD comprises one or more sgRNAs targeting exon 8 of cIL1RAP. In some embodiments, the CRISPR gene-editing system for the treatment of DDD comprises one or more sgRNAs targeting exon 9 of cIL1RAP. In some embodiments, the CRISPR gene-editing system for the treatment of DDD comprises one or more sgRNAs targeting exon 10 of cIL1RAP. In some embodiments, the CRISPR gene-editing system for the treatment of DDD comprises one or more sgRNAs targeting exon 11 of cIL1RAP. In some embodiments, the CRISPR gene-editing system for the treatment of DDD comprises one or more sgRNAs targeting exon 12 of cIL1RAP. In some embodiments, the CRISPR gene-editing system for the treatment of DDD comprises one or more sgRNAs targeting exon 13 of cIL1RAP. In some embodiments, the CRISPR gene-editing system for the treatment of DDD comprises one or more sgRNAs targeting exon 14 of cIL1RAP. In some embodiments, the CRISPR gene-editing system for the treatment of DDD comprises one or more sgRNAs targeting exon 15 of cIL1RAP. In some embodiments, the CRISPR gene-editing system for the treatment of DDD comprises one or more sgRNAs targeting exon 16 of cIL1RAP.

In some embodiments, the CRISPR gene-editing system for the treatment of DDD comprises one or more sgRNAs targeting exon 1 of eIL1RAP. In some embodiments, the CRISPR gene-editing system for the treatment of DDD comprises one or more sgRNAs targeting exon 2 of eIL1RAP. In some embodiments, the CRISPR gene-editing system for the treatment of DDD comprises one or more sgRNAs targeting exon 3 of eIL1RAP. In some embodiments, the CRISPR gene-editing system for the treatment of DDD comprises one or more sgRNAs targeting exon 4 of eIL1RAP. In some embodiments, the CRISPR gene-editing system for the treatment of DDD comprises one or more sgRNAs targeting exon 5 of eIL1RAP. In some embodiments, the CRISPR gene-editing system for the treatment of DDD comprises one or more sgRNAs targeting exon 6 of eIL1RAP. In some embodiments, the CRISPR gene-editing system for the treatment of DDD comprises one or more sgRNAs targeting exon 7 of eIL1RAP. In some embodiments, the CRISPR gene-editing system for the treatment of DDD comprises one or more sgRNAs targeting exon 8 of eIL1RAP. In some embodiments, the CRISPR gene-editing system for the treatment of DDD comprises one or more sgRNAs targeting exon 9 of eIL1RAP. In some embodiments, the CRISPR gene-editing system for the treatment of DDD comprises one or more sgRNAs targeting exon 10 of eIL1RAP. In some embodiments, the CRISPR gene-editing system for the treatment of DDD comprises one or more sgRNAs targeting exon 11 of eIL1RAP. In some embodiments, the CRISPR gene-editing system for the treatment of DDD comprises one or more sgRNAs targeting exon 12 of eIL1RAP. In some embodiments, the CRISPR gene-editing system for the treatment of DDD comprises one or more sgRNAs targeting exon 13 of eIL1RAP.

In some embodiments, the CRISPR gene-editing system for the treatment of DDD comprises one or more sgRNAs targeting exon 1 of fIL1RAP. In some embodiments, the CRISPR gene-editing system for the treatment of DDD comprises one or more sgRNAs targeting exon 2 of fIL1RAP. In some embodiments, the CRISPR gene-editing system for the treatment of DDD comprises one or more sgRNAs targeting exon 3 of fIL1RAP. In some embodiments, the CRISPR gene-editing system for the treatment of DDD comprises one or more sgRNAs targeting exon 4 of fIL1RAP. In some embodiments, the CRISPR gene-editing system for the treatment of DDD comprises one or more sgRNAs targeting exon 5 of fIL1RAP. In some embodiments, the CRISPR gene-editing system for the treatment of DDD comprises one or more sgRNAs targeting exon 6 of fIL1RAP. In some embodiments, the CRISPR gene-editing system for the treatment of DDD comprises one or more sgRNAs targeting exon 7 of fIL1RAP. In some embodiments, the CRISPR gene-editing system for the treatment of DDD comprises one or more sgRNAs targeting exon 8 of fIL1RAP. In some embodiments, the CRISPR gene-editing system for the treatment of DDD comprises one or more sgRNAs targeting exon 9 of fIL1RAP. In some embodiments, the CRISPR gene-editing system for the treatment of DDD comprises one or more sgRNAs targeting exon 10 of fIL1RAP. In some embodiments, the CRISPR gene-editing system for the treatment of DDD comprises one or more sgRNAs targeting exon 11 of fIL1RAP. In some embodiments, the CRISPR gene-editing system for the treatment of DDD comprises one or more sgRNAs targeting exon 12 of fIL1RAP. In some embodiments, the CRISPR gene-editing system for the treatment of DDD comprises one or more sgRNAs targeting exon 13 of fIL1RAP. In some embodiments, the CRISPR gene-editing system for the treatment of DDD comprises one or more sgRNAs targeting exon 14 of fIL1RAP.

In some embodiments, the CRISPR gene-editing system for the treatment of DDD comprises one or more sgRNAs targeting TGFBR1. In some embodiments, the CRISPR gene-editing system for the treatment of DDD comprises one or more sgRNAs targeting hTGFBR1. In some embodiments, the CRISPR gene-editing system for the treatment of DDD comprises one or more sgRNAs targeting cTGFBR1. In some embodiments, the CRISPR gene-editing system for the treatment of DDD comprises one or more sgRNAs targeting e TGFBR1. In some embodiments, the CRISPR gene-editing system for the treatment of DDD comprises one or more sgRNAs targeting fTGFBR1.

In some embodiments, the CRISPR gene-editing system for the treatment of DDD comprises one or more sgRNAs targeting exon 1 of hTGFBR1. In some embodiments, the CRISPR gene-editing system for the treatment of DDD comprises one or more sgRNAs targeting exon 2 of hTGFBR1. In some embodiments, the CRISPR gene-editing system for the treatment of DDD comprises one or more sgRNAs targeting exon 3 of hTGFBR1. In some embodiments, the CRISPR gene-editing system for the treatment of DDD comprises one or more sgRNAs targeting exon 4 of hTGFBR1. In some embodiments, the CRISPR gene-editing system for the treatment of DDD comprises one or more sgRNAs targeting exon 5 of hTGFBR1. In some embodiments, the CRISPR gene-editing system for the treatment of DDD comprises one or more sgRNAs targeting exon 6 of hTGFBR1. In some embodiments, the CRISPR gene-editing system for the treatment of DDD comprises one or more sgRNAs targeting exon 7 of hTGFBR1. In some embodiments, the CRISPR gene-editing system for the treatment of DDD comprises one or more sgRNAs targeting exon 8 of hTGFBR1. In some embodiments, the CRISPR gene-editing system for the treatment of DDD comprises one or more sgRNAs targeting exon 9 of hTGFBR1. In some embodiments, the CRISPR gene-editing system for the treatment of DDD comprises one or more sgRNAs targeting exon 10 of hTGFBR1. In some embodiments, the CRISPR gene-editing system for the treatment of DDD comprises one or more sgRNAs targeting exon 11 of hTGFBR1.

In some embodiments, the CRISPR gene-editing system for the treatment of DDD comprises one or more sgRNAs targeting exon 1 of cTGFBR1. In some embodiments, the CRISPR gene-editing system for the treatment of DDD comprises one or more sgRNAs targeting exon 2 of cTGFBR1. In some embodiments, the CRISPR gene-editing system for the treatment of DDD comprises one or more sgRNAs targeting exon 3 of cTGFBR1. In some embodiments, the CRISPR gene-editing system for the treatment of DDD comprises one or more sgRNAs targeting exon 4 of cTGFBR1. In some embodiments, the CRISPR gene-editing system for the treatment of DDD comprises one or more sgRNAs targeting exon 5 of cTGFBR1. In some embodiments, the CRISPR gene-editing system for the treatment of DDD comprises one or more sgRNAs targeting exon 6 of cTGFBR1. In some embodiments, the CRISPR gene-editing system for the treatment of DDD comprises one or more sgRNAs targeting exon 7 of cTGFBR1. In some embodiments, the CRISPR gene-editing system for the treatment of DDD comprises one or more sgRNAs targeting exon 8 of cTGFBR1. In some embodiments, the CRISPR gene-editing system for the treatment of DDD comprises one or more sgRNAs targeting exon 9 of cTGFBR1.

In some embodiments, the CRISPR gene-editing system for the treatment of DDD comprises one or more sgRNAs targeting exon 1 of eTGFBR1. In some embodiments, the CRISPR gene-editing system for the treatment of DDD comprises one or more sgRNAs targeting exon 2 of eTGFBR1. In some embodiments, the CRISPR gene-editing system for the treatment of DDD comprises one or more sgRNAs targeting exon 3 of eTGFBR1. In some embodiments, the CRISPR gene-editing system for the treatment of DDD comprises one or more sgRNAs targeting exon 4 of eTGFBR1. In some embodiments, the CRISPR gene-editing system for the treatment of DDD comprises one or more sgRNAs targeting exon 5 of eTGFBR1. In some embodiments, the CRISPR gene-editing system for the treatment of DDD comprises one or more sgRNAs targeting exon 6 of eTGFBR1. In some embodiments, the CRISPR gene-editing system for the treatment of DDD comprises one or more sgRNAs targeting exon 7 of eTGFBR1. In some embodiments, the CRISPR gene-editing system for the treatment of DDD comprises one or more sgRNAs targeting exon 8 of eTGFBR1. In some embodiments, the CRISPR gene-editing system for the treatment of DDD comprises one or more sgRNAs targeting exon 9 of eTGFBR1. In some embodiments, the CRISPR gene-editing system for the treatment of DDD comprises one or more sgRNAs targeting exon 10 of eTGFBR1. In some embodiments, the CRISPR gene-editing system for the treatment of DDD comprises one or more sgRNAs targeting exon 11 of eTGFBR1. In some embodiments, the CRISPR gene-editing system for the treatment of DDD comprises one or more sgRNAs targeting exon 12 of eTGFBR1.

In some embodiments, the CRISPR gene-editing system for the treatment of DDD comprises one or more sgRNAs targeting exon 1 of fTGFBR1. In some embodiments, the CRISPR gene-editing system for the treatment of DDD comprises one or more sgRNAs targeting exon 2 of fTGFBR1. In some embodiments, the CRISPR gene-editing system for the treatment of DDD comprises one or more sgRNAs targeting exon 3 of fTGFBR1. In some embodiments, the CRISPR gene-editing system for the treatment of DDD comprises one or more sgRNAs targeting exon 4 of fTGFBR1. In some embodiments, the CRISPR gene-editing system for the treatment of DDD comprises one or more sgRNAs targeting exon 5 of fTGFBR1. In some embodiments, the CRISPR gene-editing system for the treatment of DDD comprises one or more sgRNAs targeting exon 6 of fTGFBR1. In some embodiments, the CRISPR gene-editing system for the treatment of DDD comprises one or more sgRNAs targeting exon 7 of fTGFBR1. In some embodiments, the CRISPR gene-editing system for the treatment of DDD comprises one or more sgRNAs targeting exon 8 of fTGFBR1. In some embodiments, the CRISPR gene-editing system for the treatment of DDD comprises one or more sgRNAs targeting exon 9 of fTGFBR1. In some embodiments, the CRISPR gene-editing system for the treatment of DDD comprises one or more sgRNAs targeting exon 10 of fTGFBR1. In some embodiments, the CRISPR gene-editing system for the treatment of DDD comprises one or more sgRNAs targeting exon 11 of fTGFBR1.

In some embodiments, the CRISPR gene-editing system for the treatment of DDD comprises one or more sgRNAs targeting TGFBR2. In some embodiments, the CRISPR gene-editing system for the treatment of DDD comprises one or more sgRNAs targeting hTGFBR2. In some embodiments, the CRISPR gene-editing system for the treatment of DDD comprises one or more sgRNAs targeting cTGFBR2. In some embodiments, the CRISPR gene-editing system for the treatment of DDD comprises one or more sgRNAs targeting eTGFBR2. In some embodiments, the CRISPR gene-editing system for the treatment of DDD comprises one or more sgRNAs targeting fTGFBR2.

In some embodiments, the CRISPR gene-editing system for the treatment of DDD comprises one or more sgRNAs targeting exon 1 of hTGFBR2. In some embodiments, the CRISPR gene-editing system for the treatment of DDD comprises one or more sgRNAs targeting exon 2 of hTGFBR2. In some embodiments, the CRISPR gene-editing system for the treatment of DDD comprises one or more sgRNAs targeting exon 3 of hTGFBR2. In some embodiments, the CRISPR gene-editing system for the treatment of DDD comprises one or more sgRNAs targeting exon 4 of hTGFBR2. In some embodiments, the CRISPR gene-editing system for the treatment of DDD comprises one or more sgRNAs targeting exon 5 of hTGFBR2. In some embodiments, the CRISPR gene-editing system for the treatment of DDD comprises one or more sgRNAs targeting exon 6 of hTGFBR2. In some embodiments, the CRISPR gene-editing system for the treatment of DDD comprises one or more sgRNAs targeting exon 7 of hTGFBR2. In some embodiments, the CRISPR gene-editing system for the treatment of DDD comprises one or more sgRNAs targeting exon 8 of hTGFBR2. In some embodiments, the CRISPR gene-editing system for the treatment of DDD comprises one or more sgRNAs targeting exon 9 of hTGFBR2.

In some embodiments, the CRISPR gene-editing system for the treatment of DDD comprises one or more sgRNAs targeting exon 1 of cTGFBR2. In some embodiments, the CRISPR gene-editing system for the treatment of DDD comprises one or more sgRNAs targeting exon 2 of cTGFBR2. In some embodiments, the CRISPR gene-editing system for the treatment of DDD comprises one or more sgRNAs targeting exon 3 of cTGFBR2. In some embodiments, the CRISPR gene-editing system for the treatment of DDD comprises one or more sgRNAs targeting exon 4 of cTGFBR2. In some embodiments, the CRISPR gene-editing system for the treatment of DDD comprises one or more sgRNAs targeting exon 5 of cTGFBR2. In some embodiments, the CRISPR gene-editing system for the treatment of DDD comprises one or more sgRNAs targeting exon 6 of cTGFBR2. In some embodiments, the CRISPR gene-editing system for the treatment of DDD comprises one or more sgRNAs targeting exon 7 of cTGFBR2. In some embodiments, the CRISPR gene-editing system for the treatment of DDD comprises one or more sgRNAs targeting exon 8 of cTGFBR2.

In some embodiments, the CRISPR gene-editing system for the treatment of DDD comprises one or more sgRNAs targeting exon 1 of eTGFBR2. In some embodiments, the CRISPR gene-editing system for the treatment of DDD comprises one or more sgRNAs targeting exon 2 of eTGFBR2. In some embodiments, the CRISPR gene-editing system for the treatment of DDD comprises one or more sgRNAs targeting exon 3 of eTGFBR2. In some embodiments, the CRISPR gene-editing system for the treatment of DDD comprises one or more sgRNAs targeting exon 4 of eTGFBR2. In some embodiments, the CRISPR gene-editing system for the treatment of DDD comprises one or more sgRNAs targeting exon 5 of eTGFBR2. In some embodiments, the CRISPR gene-editing system for the treatment of DDD comprises one or more sgRNAs targeting exon 6 of eTGFBR2. In some embodiments, the CRISPR gene-editing system for the treatment of DDD comprises one or more sgRNAs targeting exon 7 of eTGFBR2. In some embodiments, the CRISPR gene-editing system for the treatment of DDD comprises one or more sgRNAs targeting exon 8 of eTGFBR2. In some embodiments, the CRISPR gene-editing system for the treatment of DDD comprises one or more sgRNAs targeting exon 9 of eTGFBR2. In some embodiments, the CRISPR gene-editing system for the treatment of DDD comprises one or more sgRNAs targeting exon 10 of eTGFBR2.

In some embodiments, the CRISPR gene-editing system for the treatment of DDD comprises one or more sgRNAs targeting exon 1 of fTGFBR2. In some embodiments, the CRISPR gene-editing system for the treatment of DDD comprises one or more sgRNAs targeting exon 2 of fTGFBR2. In some embodiments, the CRISPR gene-editing system for the treatment of DDD comprises one or more sgRNAs targeting exon 3 of fTGFBR2. In some embodiments, the CRISPR gene-editing system for the treatment of DDD comprises one or more sgRNAs targeting exon 4 of fTGFBR2. In some embodiments, the CRISPR gene-editing system for the treatment of DDD comprises one or more sgRNAs targeting exon 5 of fTGFBR2. In some embodiments, the CRISPR gene-editing system for the treatment of DDD comprises one or more sgRNAs targeting exon 6 of fTGFBR2. In some embodiments, the CRISPR gene-editing system for the treatment of DDD comprises one or more sgRNAs targeting exon 7 of fTGFBR2. In some embodiments, the CRISPR gene-editing system for the treatment of DDD comprises one or more sgRNAs targeting exon 8 of fTGFBR2.

In some embodiments, the CRISPR gene-editing system for the treatment of DDD comprises one or more sgRNAs targeting IL6R. In some embodiments, the CRISPR gene-editing system for the treatment of DDD comprises one or more sgRNAs targeting hIL6R. In some embodiments, the CRISPR gene-editing system for the treatment of DDD comprises one or more sgRNAs targeting cIL6R. In some embodiments, the CRISPR gene-editing system for the treatment of DDD comprises one or more sgRNAs targeting eIL6R. In some embodiments, the CRISPR gene-editing system for the treatment of DDD comprises one or more sgRNAs targeting fIL6R.

In some embodiments, the CRISPR gene-editing system for the treatment of DDD comprises one or more sgRNAs targeting exon 1 of hIL6R. In some embodiments, the CRISPR gene-editing system for the treatment of DDD comprises one or more sgRNAs targeting exon 2 of hIL6R. In some embodiments, the CRISPR gene-editing system for the treatment of DDD comprises one or more sgRNAs targeting exon 3 of hIL6R. In some embodiments, the CRISPR gene-editing system for the treatment of DDD comprises one or more sgRNAs targeting exon 4 of hIL6R. In some embodiments, the CRISPR gene-editing system for the treatment of DDD comprises one or more sgRNAs targeting exon 5 of hIL6R. In some embodiments, the CRISPR gene-editing system for the treatment of DDD comprises one or more sgRNAs targeting exon 6 of hIL6R. In some embodiments, the CRISPR gene-editing system for the treatment of DDD comprises one or more sgRNAs targeting exon 7 of hIL6R. In some embodiments, the CRISPR gene-editing system for the treatment of DDD comprises one or more sgRNAs targeting exon 8 of hIL6R. In some embodiments, the CRISPR gene-editing system for the treatment of DDD comprises one or more sgRNAs targeting exon 9 of hIL6R. In some embodiments, the CRISPR gene-editing system for the treatment of DDD comprises one or more sgRNAs targeting exon 10 of hIL6R. In some embodiments, the CRISPR gene-editing system for the treatment of DDD comprises one or more sgRNAs targeting exon 11 of hIL6R In some embodiments, the CRISPR gene-editing system for the treatment of DDD comprises one or more sgRNAs targeting exon 12 of hIL6R. In some embodiments, the CRISPR gene-editing system for the treatment of DDD comprises one or more sgRNAs targeting exon 13 of hIL6R. In some embodiments, the CRISPR gene-editing system for the treatment of DDD comprises one or more sgRNAs targeting exon 14 of hIL6R. In some embodiments, the CRISPR gene-editing system for the treatment of DDD comprises one or more sgRNAs targeting exon 15 of hIL6R.

In some embodiments, the CRISPR gene-editing system for the treatment of DDD comprises one or more sgRNAs targeting exon 1 of cIL6R. In some embodiments, the CRISPR gene-editing system for the treatment of DDD comprises one or more sgRNAs targeting exon 2 of cIL6R. In some embodiments, the CRISPR gene-editing system for the treatment of DDD comprises one or more sgRNAs targeting exon 3 of cIL6R. In some embodiments, the CRISPR gene-editing system for the treatment of DDD comprises one or more sgRNAs targeting exon 4 of cIL6R. In some embodiments, the CRISPR gene-editing system for the treatment of DDD comprises one or more sgRNAs targeting exon 5 of cIL6R. In some embodiments, the CRISPR gene-editing system for the treatment of DDD comprises one or more sgRNAs targeting exon 6 of cIL6R. In some embodiments, the CRISPR gene-editing system for the treatment of DDD comprises one or more sgRNAs targeting exon 7 of cIL6R. In some embodiments, the CRISPR gene-editing system for the treatment of DDD comprises one or more sgRNAs targeting exon 8 of cIL6R. In some embodiments, the CRISPR gene-editing system for the treatment of DDD comprises one or more sgRNAs targeting exon 9 of cIL6R. In some embodiments, the CRISPR gene-editing system for the treatment of DDD comprises one or more sgRNAs targeting exon 10 of cIL6R. In some embodiments, the CRISPR gene-editing system for the treatment of DDD comprises one or more sgRNAs targeting exon 11 of cIL6R. In some embodiments, the CRISPR gene-editing system for the treatment of DDD comprises one or more sgRNAs targeting exon 12 of cIL6R. In some embodiments, the CRISPR gene-editing system for the treatment of DDD comprises one or more sgRNAs targeting exon 13 of cIL6R.

In some embodiments, the CRISPR gene-editing system for the treatment of DDD comprises one or more sgRNAs targeting exon 1 of eIL6R. In some embodiments, the CRISPR gene-editing system for the treatment of DDD comprises one or more sgRNAs targeting exon 2 of eIL6R. In some embodiments, the CRISPR gene-editing system for the treatment of DDD comprises one or more sgRNAs targeting exon 3 of eIL6R. In some embodiments, the CRISPR gene-editing system for the treatment of DDD comprises one or more sgRNAs targeting exon 4 of eIL6R. In some embodiments, the CRISPR gene-editing system for the treatment of DDD comprises one or more sgRNAs targeting exon 5 of eIL6R. In some embodiments, the CRISPR gene-editing system for the treatment of DDD comprises one or more sgRNAs targeting exon 6 of eIL6R. In some embodiments, the CRISPR gene-editing system for the treatment of DDD comprises one or more sgRNAs targeting exon 7 of eIL6R. In some embodiments, the CRISPR gene-editing system for the treatment of DDD comprises one or more sgRNAs targeting exon 8 of eIL6R. In some embodiments, the CRISPR gene-editing system for the treatment of DDD comprises one or more sgRNAs targeting exon 9 of eIL6R. In some embodiments, the CRISPR gene-editing system for the treatment of DDD comprises one or more sgRNAs targeting exon 10 of eIL6R. In some embodiments, the CRISPR gene-editing system for the treatment of DDD comprises one or more sgRNAs targeting exon 11 of eIL6R.

In some embodiments, the CRISPR gene-editing system for the treatment of DDD comprises one or more sgRNAs targeting exon 1 of fIL6R. In some embodiments, the CRISPR gene-editing system for the treatment of DDD comprises one or more sgRNAs targeting exon 2 of fIL6R. In some embodiments, the CRISPR gene-editing system for the treatment of DDD comprises one or more sgRNAs targeting exon 3 of fIL6R. In some embodiments, the CRISPR gene-editing system for the treatment of DDD comprises one or more sgRNAs targeting exon 4 of fIL6R. In some embodiments, the CRISPR gene-editing system for the treatment of DDD comprises one or more sgRNAs targeting exon 5 of fIL6R. In some embodiments, the CRISPR gene-editing system for the treatment of DDD comprises one or more sgRNAs targeting exon 6 of fIL6R. In some embodiments, the CRISPR gene-editing system for the treatment of DDD comprises one or more sgRNAs targeting exon 7 of fIL6R. In some embodiments, the CRISPR gene-editing system for the treatment of DDD comprises one or more sgRNAs targeting exon 8 of fIL6R. In some embodiments, the CRISPR gene-editing system for the treatment of DDD comprises one or more sgRNAs targeting exon 9 of fIL6R. In some embodiments, the CRISPR gene-editing system for the treatment of DDD comprises one or more sgRNAs targeting exon 10 of fIL6R.

In some embodiments, the CRISPR gene-editing system for the treatment of DDD comprises one or more sgRNAs targeting IL6ST. In some embodiments, the CRISPR gene-editing system for the treatment of DDD comprises one or more sgRNAs targeting hIL6ST. In some embodiments, the CRISPR gene-editing system for the treatment of DDD comprises one or more sgRNAs targeting cIL6ST. In some embodiments, the CRISPR gene-editing system for the treatment of DDD comprises one or more sgRNAs targeting eIL6ST. In some embodiments, the CRISPR gene-editing system for the treatment of DDD comprises one or more sgRNAs targeting fIL6ST.

In some embodiments, the CRISPR gene-editing system for the treatment of DDD comprises one or more sgRNAs targeting exon 1 of hIL6ST. In some embodiments, the CRISPR gene-editing system for the treatment of DDD comprises one or more sgRNAs targeting exon 2 of hIL6ST. In some embodiments, the CRISPR gene-editing system for the treatment of DDD comprises one or more sgRNAs targeting exon 3 of hIL6ST. In some embodiments, the CRISPR gene-editing system for the treatment of DDD comprises one or more sgRNAs targeting exon 4 of hIL6ST. In some embodiments, the CRISPR gene-editing system for the treatment of DDD comprises one or more sgRNAs targeting exon 5 of hIL6ST. In some embodiments, the CRISPR gene-editing system for the treatment of DDD comprises one or more sgRNAs targeting exon 6 of hIL6ST. In some embodiments, the CRISPR gene-editing system for the treatment of DDD comprises one or more sgRNAs targeting exon 7 of hIL6ST. In some embodiments, the CRISPR gene-editing system for the treatment of DDD comprises one or more sgRNAs targeting exon 8 of hIL6ST. In some embodiments, the CRISPR gene-editing system for the treatment of DDD comprises one or more sgRNAs targeting exon 9 of hIL6ST. In some embodiments, the CRISPR gene-editing system for the treatment of DDD comprises one or more sgRNAs targeting exon 10 of hIL6ST. In some embodiments, the CRISPR gene-editing system for the treatment of DDD comprises one or more sgRNAs targeting exon 11 of hIL6ST. In some embodiments, the CRISPR gene-editing system for the treatment of DDD comprises one or more sgRNAs targeting exon 12 of hIL6ST. In some embodiments, the CRISPR gene-editing system for the treatment of DDD comprises one or more sgRNAs targeting exon 13 of hIL6ST. In some embodiments, the CRISPR gene-editing system for the treatment of DDD comprises one or more sgRNAs targeting exon 14 of hIL6ST. In some embodiments, the CRISPR gene-editing system for the treatment of DDD comprises one or more sgRNAs targeting exon 15 of hIL6ST. In some embodiments, the CRISPR gene-editing system for the treatment of DDD comprises one or more sgRNAs targeting exon 16 of hIL6ST. In some embodiments, the CRISPR gene-editing system for the treatment of DDD comprises one or more sgRNAs targeting exon 17 of hIL6ST. In some embodiments, the CRISPR gene-editing system for the treatment of DDD comprises one or more sgRNAs targeting exon 18 of hIL6ST. In some embodiments, the CRISPR gene-editing system for the treatment of DDD comprises one or more sgRNAs targeting exon 19 of hIL6ST.

In some embodiments, the CRISPR gene-editing system for the treatment of DDD comprises one or more sgRNAs targeting exon 1 of cIL6ST. In some embodiments, the CRISPR gene-editing system for the treatment of DDD comprises one or more sgRNAs targeting exon 2 of cIL6ST. In some embodiments, the CRISPR gene-editing system for the treatment of DDD comprises one or more sgRNAs targeting exon 3 of cIL6ST. In some embodiments, the CRISPR gene-editing system for the treatment of DDD comprises one or more sgRNAs targeting exon 4 of cIL6ST. In some embodiments, the CRISPR gene-editing system for the treatment of DDD comprises one or more sgRNAs targeting exon 5 of cIL6ST. In some embodiments, the CRISPR gene-editing system for the treatment of DDD comprises one or more sgRNAs targeting exon 6 of cIL6ST. In some embodiments, the CRISPR gene-editing system for the treatment of DDD comprises one or more sgRNAs targeting exon 7 of cIL6ST. In some embodiments, the CRISPR gene-editing system for the treatment of DDD comprises one or more sgRNAs targeting exon 8 of cIL6ST. In some embodiments, the CRISPR gene-editing system for the treatment of DDD comprises one or more sgRNAs targeting exon 9 of cIL6ST. In some embodiments, the CRISPR gene-editing system for the treatment of DDD comprises one or more sgRNAs targeting exon 10 of cIL6ST. In some embodiments, the CRISPR gene-editing system for the treatment of DDD comprises one or more sgRNAs targeting exon 11 of cIL6ST In some embodiments, the CRISPR gene-editing system for the treatment of DDD comprises one or more sgRNAs targeting exon 12 of cIL6ST. In some embodiments, the CRISPR gene-editing system for the treatment of DDD comprises one or more sgRNAs targeting exon 13 of cIL6ST. In some embodiments, the CRISPR gene-editing system for the treatment of DDD comprises one or more sgRNAs targeting exon 14 of cIL6ST. In some embodiments, the CRISPR gene-editing system for the treatment of DDD comprises one or more sgRNAs targeting exon 15 of cIL6ST. In some embodiments, the CRISPR gene-editing system for the treatment of DDD comprises one or more sgRNAs targeting exon 16 of cIL6ST. In some embodiments, the CRISPR gene-editing system for the treatment of DDD comprises one or more sgRNAs targeting exon 17 of cIL6ST. In some embodiments, the CRISPR gene-editing system for the treatment of DDD comprises one or more sgRNAs targeting exon 18 of cIL6ST.

In some embodiments, the CRISPR gene-editing system for the treatment of DDD comprises one or more sgRNAs targeting exon 1 of eIL6ST. In some embodiments, the CRISPR gene-editing system for the treatment of DDD comprises one or more sgRNAs targeting exon 2 of eIL6ST. In some embodiments, the CRISPR gene-editing system for the treatment of DDD comprises one or more sgRNAs targeting exon 3 of eIL6ST. In some embodiments, the CRISPR gene-editing system for the treatment of DDD comprises one or more sgRNAs targeting exon 4 of eIL6ST. In some embodiments, the CRISPR gene-editing system for the treatment of DDD comprises one or more sgRNAs targeting exon 5 of eIL6ST. In some embodiments, the CRISPR gene-editing system for the treatment of DDD comprises one or more sgRNAs targeting exon 6 of eIL6ST. In some embodiments, the CRISPR gene-editing system for the treatment of DDD comprises one or more sgRNAs targeting exon 7 of eIL6ST. In some embodiments, the CRISPR gene-editing system for the treatment of DDD comprises one or more sgRNAs targeting exon 8 of eIL6ST. In some embodiments, the CRISPR gene-editing system for the treatment of DDD comprises one or more sgRNAs targeting exon 9 of eIL6ST. In some embodiments, the CRISPR gene-editing system for the treatment of DDD comprises one or more sgRNAs targeting exon 10 of eIL6ST. In some embodiments, the CRISPR gene-editing system for the treatment of DDD comprises one or more sgRNAs targeting exon 11 of eIL6ST In some embodiments, the CRISPR gene-editing system for the treatment of DDD comprises one or more sgRNAs targeting exon 12 of eIL6ST. In some embodiments, the CRISPR gene-editing system for the treatment of DDD comprises one or more sgRNAs targeting exon 13 of eIL6ST. In some embodiments, the CRISPR gene-editing system for the treatment of DDD comprises one or more sgRNAs targeting exon 14 of eIL6ST. In some embodiments, the CRISPR gene-editing system for the treatment of DDD comprises one or more sgRNAs targeting exon 15 of eIL6ST. In some embodiments, the CRISPR gene-editing system for the treatment of DDD comprises one or more sgRNAs targeting exon 16 of eIL6ST. In some embodiments, the CRISPR gene-editing system for the treatment of DDD comprises one or more sgRNAs targeting exon 17 of eIL6ST. In some embodiments, the CRISPR gene-editing system for the treatment of DDD comprises one or more sgRNAs targeting exon 18 of eIL6ST.

In some embodiments, the CRISPR gene-editing system for the treatment of DDD comprises one or more sgRNAs targeting exon 1 of fIL6ST. In some embodiments, the CRISPR gene-editing system for the treatment of DDD comprises one or more sgRNAs targeting exon 2 of fIL6ST. In some embodiments, the CRISPR gene-editing system for the treatment of DDD comprises one or more sgRNAs targeting exon 3 of fIL6ST. In some embodiments, the CRISPR gene-editing system for the treatment of DDD comprises one or more sgRNAs targeting exon 4 of fIL6ST. In some embodiments, the CRISPR gene-editing system for the treatment of DDD comprises one or more sgRNAs targeting exon 5 of fIL6ST. In some embodiments, the CRISPR gene-editing system for the treatment of DDD comprises one or more sgRNAs targeting exon 6 of fIL6ST. In some embodiments, the CRISPR gene-editing system for the treatment of DDD comprises one or more sgRNAs targeting exon 7 of fIL6ST. In some embodiments, the CRISPR gene-editing system for the treatment of DDD comprises one or more sgRNAs targeting exon 8 of fIL6ST. In some embodiments, the CRISPR gene-editing system for the treatment of DDD comprises one or more sgRNAs targeting exon 9 of fIL6ST. In some embodiments, the CRISPR gene-editing system for the treatment of DDD comprises one or more sgRNAs targeting exon 10 of fIL6ST. In some embodiments, the CRISPR gene-editing system for the treatment of DDD comprises one or more sgRNAs targeting exon 11 of fIL6ST In some embodiments, the CRISPR gene-editing system for the treatment of DDD comprises one or more sgRNAs targeting exon 12 of fIL6ST. In some embodiments, the CRISPR gene-editing system for the treatment of DDD comprises one or more sgRNAs targeting exon 13 of fIL6ST. In some embodiments, the CRISPR gene-editing system for the treatment of DDD comprises one or more sgRNAs targeting exon 14 of fIL6ST. In some embodiments, the CRISPR gene-editing system for the treatment of DDD comprises one or more sgRNAs targeting exon 15 of fIL6ST. In some embodiments, the CRISPR gene-editing system for the treatment of DDD comprises one or more sgRNAs targeting exon 16 of fIL6ST. In some embodiments, the CRISPR gene-editing system for the treatment of DDD comprises one or more sgRNAs targeting exon 17 of fIL6ST. In some embodiments, the CRISPR gene-editing system for the treatment of DDD comprises one or more sgRNAs targeting exon 18 of fIL6ST. In some embodiments, the CRISPR gene-editing system for the treatment of DDD comprises one or more sgRNAs targeting exon 19 of fIL6ST. In some embodiments, the CRISPR gene-editing system for the treatment of DDD comprises one or more sgRNAs targeting exon 20 of fIL6ST.

In some embodiments, the CRISPR gene-editing system for the treatment of DDD comprises one or more sgRNAs targeting TNFRSF1A. In some embodiments, the CRISPR gene-editing system for the treatment of DDD comprises one or more sgRNAs targeting hTNFRSF1A. In some embodiments, the CRISPR gene-editing system for the treatment of DDD comprises one or more sgRNAs targeting cTNFRSF1A. In some embodiments, the CRISPR gene-editing system for the treatment of DDD comprises one or more sgRNAs targeting eTNFRSF1A. In some embodiments, the CRISPR gene-editing system for the treatment of DDD comprises one or more sgRNAs targeting fTNFRSF1A.

In some embodiments, the CRISPR gene-editing system for the treatment of DDD comprises one or more sgRNAs targeting exon 1 of hTNFRSF1A. In some embodiments, the CRISPR gene-editing system for the treatment of DDD comprises one or more sgRNAs targeting exon 2 of hTNFRSF1A. In some embodiments, the CRISPR gene-editing system for the treatment of DDD comprises one or more sgRNAs targeting exon 3 of hTNFRSF1A. In some embodiments, the CRISPR gene-editing system for the treatment of DDD comprises one or more sgRNAs targeting exon 4 of hTNFRSF1A. In some embodiments, the CRISPR gene-editing system for the treatment of DDD comprises one or more sgRNAs targeting exon 5 of hTNFRSF1A. In some embodiments, the CRISPR gene-editing system for the treatment of DDD comprises one or more sgRNAs targeting exon 6 of hTNFRSF1A. In some embodiments, the CRISPR gene-editing system for the treatment of DDD comprises one or more sgRNAs targeting exon 7 of hTNFRSF1A. In some embodiments, the CRISPR gene-editing system for the treatment of DDD comprises one or more sgRNAs targeting exon 8 of hTNFRSF1A. In some embodiments, the CRISPR gene-editing system for the treatment of DDD comprises one or more sgRNAs targeting exon 9 of hTNFRSF1A. In some embodiments, the CRISPR gene-editing system for the treatment of DDD comprises one or more sgRNAs targeting exon 10 of hTNFRSF1A. In some embodiments, the CRISPR gene-editing system for the treatment of DDD comprises one or more sgRNAs targeting exon 11 of hTNFRSF1A.

In some embodiments, the CRISPR gene-editing system for the treatment of DDD comprises one or more sgRNAs targeting exon 1 of cTNFRSF1A. In some embodiments, the CRISPR gene-editing system for the treatment of DDD comprises one or more sgRNAs targeting exon 2 of cTNFRSF1A. In some embodiments, the CRISPR gene-editing system for the treatment of DDD comprises one or more sgRNAs targeting exon 3 of cTNFRSF1A. In some embodiments, the CRISPR gene-editing system for the treatment of DDD comprises one or more sgRNAs targeting exon 4 of cTNFRSF1A. In some embodiments, the CRISPR gene-editing system for the treatment of DDD comprises one or more sgRNAs targeting exon 5 of cTNFRSF1A. In some embodiments, the CRISPR gene-editing system for the treatment of DDD comprises one or more sgRNAs targeting exon 6 of cTNFRSF1A. In some embodiments, the CRISPR gene-editing system for the treatment of DDD comprises one or more sgRNAs targeting exon 7 of cTNFRSF1A. In some embodiments, the CRISPR gene-editing system for the treatment of DDD comprises one or more sgRNAs targeting exon 8 of cTNFRSF1A. In some embodiments, the CRISPR gene-editing system for the treatment of DDD comprises one or more sgRNAs targeting exon 9 of cTNFRSF1A. In some embodiments, the CRISPR gene-editing system for the treatment of DDD comprises one or more sgRNAs targeting exon 10 of cTNFRSF1A. In some embodiments, the CRISPR gene-editing system for the treatment of DDD comprises one or more sgRNAs targeting exon 11 of cTNFRSF1A.

In some embodiments, the CRISPR gene-editing system for the treatment of DDD comprises one or more sgRNAs targeting exon 1 of eTNFRSF1A. In some embodiments, the CRISPR gene-editing system for the treatment of DDD comprises one or more sgRNAs targeting exon 2 of eTNFRSF1A. In some embodiments, the CRISPR gene-editing system for the treatment of DDD comprises one or more sgRNAs targeting exon 3 of eTNFRSF1A. In some embodiments, the CRISPR gene-editing system for the treatment of DDD comprises one or more sgRNAs targeting exon 4 of eTNFRSF1A. In some embodiments, the CRISPR gene-editing system for the treatment of DDD comprises one or more sgRNAs targeting exon 5 of eTNFRSF1A. In some embodiments, the CRISPR gene-editing system for the treatment of DDD comprises one or more sgRNAs targeting exon 6 of eTNFRSF1A. In some embodiments, the CRISPR gene-editing system for the treatment of DDD comprises one or more sgRNAs targeting exon 7 of eTNFRSF1A. In some embodiments, the CRISPR gene-editing system for the treatment of DDD comprises one or more sgRNAs targeting exon 8 of eTNFRSF1A. In some embodiments, the CRISPR gene-editing system for the treatment of DDD comprises one or more sgRNAs targeting exon 9 of eTNFRSF1A. In some embodiments, the CRISPR gene-editing system for the treatment of DDD comprises one or more sgRNAs targeting exon 10 of eTNFRSF1A.

In some embodiments, the CRISPR gene-editing system for the treatment of DDD comprises one or more sgRNAs targeting exon 1 of fTNFRSF1A. In some embodiments, the CRISPR gene-editing system for the treatment of DDD comprises one or more sgRNAs targeting exon 2 of fTNFRSF1A. In some embodiments, the CRISPR gene-editing system for the treatment of DDD comprises one or more sgRNAs targeting exon 3 of fTNFRSF1A. In some embodiments, the CRISPR gene-editing system for the treatment of DDD comprises one or more sgRNAs targeting exon 4 of fTNFRSF1A. In some embodiments, the CRISPR gene-editing system for the treatment of DDD comprises one or more sgRNAs targeting exon 5 of fTNFRSF1A. In some embodiments, the CRISPR gene-editing system for the treatment of DDD comprises one or more sgRNAs targeting exon 6 of fTNFRSF1A. In some embodiments, the CRISPR gene-editing system for the treatment of DDD comprises one or more sgRNAs targeting exon 7 of fTNFRSF1A. In some embodiments, the CRISPR gene-editing system for the treatment of DDD comprises one or more sgRNAs targeting exon 8 of fTNFRSF1A. In some embodiments, the CRISPR gene-editing system for the treatment of DDD comprises one or more sgRNAs targeting exon 9 of fTNFRSF1A. In some embodiments, the CRISPR gene-editing system for the treatment of DDD comprises one or more sgRNAs targeting exon 10 of fTNFRSF1A.

In some embodiments, the CRISPR gene-editing system for the treatment of DDD comprises one or more sgRNAs targeting TNFRSF1B. In some embodiments, the CRISPR gene-editing system for the treatment of DDD comprises one or more sgRNAs targeting hTNFRSF1B. In some embodiments, the CRISPR gene-editing system for the treatment of DDD comprises one or more sgRNAs targeting cTNFRSF1B. In some embodiments, the CRISPR gene-editing system for the treatment of DDD comprises one or more sgRNAs targeting eTNFRSF1B. In some embodiments, the CRISPR gene-editing system for the treatment of DDD comprises one or more sgRNAs targeting fTNFRSF1B.

In some embodiments, the CRISPR gene-editing system for the treatment of DDD comprises one or more sgRNAs targeting exon 1 of hTNFRSF1B. In some embodiments, the CRISPR gene-editing system for the treatment of DDD comprises one or more sgRNAs targeting exon 2 of hTNFRSF1B. In some embodiments, the CRISPR gene-editing system for the treatment of DDD comprises one or more sgRNAs targeting exon 3 of hTNFRSF1B. In some embodiments, the CRISPR gene-editing system for the treatment of DDD comprises one or more sgRNAs targeting exon 4 of hTNFRSF1B. In some embodiments, the CRISPR gene-editing system for the treatment of DDD comprises one or more sgRNAs targeting exon 5 of hTNFRSF1B. In some embodiments, the CRISPR gene-editing system for the treatment of DDD comprises one or more sgRNAs targeting exon 6 of hTNFRSF1B. In some embodiments, the CRISPR gene-editing system for the treatment of DDD comprises one or more sgRNAs targeting exon 7 of hTNFRSF1B. In some embodiments, the CRISPR gene-editing system for the treatment of DDD comprises one or more sgRNAs targeting exon 8 of hTNFRSF1B. In some embodiments, the CRISPR gene-editing system for the treatment of DDD comprises one or more sgRNAs targeting exon 9 of hTNFRSF1B. In some embodiments, the CRISPR gene-editing system for the treatment of DDD comprises one or more sgRNAs targeting exon 10 of hTNFRSF1B. In some embodiments, the CRISPR gene-editing system for the treatment of DDD comprises one or more sgRNAs targeting exon 11 of hTNFRSF1B In some embodiments, the CRISPR gene-editing system for the treatment of DDD comprises one or more sgRNAs targeting exon 12 of hTNFRSF1B. In some embodiments, the CRISPR gene-editing system for the treatment of DDD comprises one or more sgRNAs targeting exon 13 of hTNFRSF1B.

In some embodiments, the CRISPR gene-editing system for the treatment of DDD comprises one or more sgRNAs targeting exon 1 of cTNFRSF1B. In some embodiments, the CRISPR gene-editing system for the treatment of DDD comprises one or more sgRNAs targeting exon 2 of cTNFRSF1B. In some embodiments, the CRISPR gene-editing system for the treatment of DDD comprises one or more sgRNAs targeting exon 3 of cTNFRSF1B. In some embodiments, the CRISPR gene-editing system for the treatment of DDD comprises one or more sgRNAs targeting exon 4 of cTNFRSF1B. In some embodiments, the CRISPR gene-editing system for the treatment of DDD comprises one or more sgRNAs targeting exon 5 of cTNFRSF1B. In some embodiments, the CRISPR gene-editing system for the treatment of DDD comprises one or more sgRNAs targeting exon 6 of cTNFRSF1B. In some embodiments, the CRISPR gene-editing system for the treatment of DDD comprises one or more sgRNAs targeting exon 7 of cTNFRSF1B. In some embodiments, the CRISPR gene-editing system for the treatment of DDD comprises one or more sgRNAs targeting exon 8 of CTNFRSF1B. In some embodiments, the CRISPR gene-editing system for the treatment of DDD comprises one or more sgRNAs targeting exon 9 of cTNFRSF1B. In some embodiments, the CRISPR gene-editing system for the treatment of DDD comprises one or more sgRNAs targeting exon 10 of cTNFRSF1B. In some embodiments, the CRISPR gene-editing system for the treatment of DDD comprises one or more sgRNAs targeting exon 11 of cTNFRSF1B.

In some embodiments, the CRISPR gene-editing system for the treatment of DDD comprises one or more sgRNAs targeting exon 1 of eTNFRSF1B. In some embodiments, the CRISPR gene-editing system for the treatment of DDD comprises one or more sgRNAs targeting exon 2 of eTNFRSF1B. In some embodiments, the CRISPR gene-editing system for the treatment of DDD comprises one or more sgRNAs targeting exon 3 of eTNFRSF1B. In some embodiments, the CRISPR gene-editing system for the treatment of DDD comprises one or more sgRNAs targeting exon 4 of eTNFRSF1B. In some embodiments, the CRISPR gene-editing system for the treatment of DDD comprises one or more sgRNAs targeting exon 5 of eTNFRSF1B. In some embodiments, the CRISPR gene-editing system for the treatment of DDD comprises one or more sgRNAs targeting exon 6 of eTNFRSF1B. In some embodiments, the CRISPR gene-editing system for the treatment of DDD comprises one or more sgRNAs targeting exon 7 of eTNFRSF1B. In some embodiments, the CRISPR gene-editing system for the treatment of DDD comprises one or more sgRNAs targeting exon 8 of eTNFRSF1B. In some embodiments, the CRISPR gene-editing system for the treatment of.

DDD comprises one or more sgRNAs targeting exon 9 of eTNFRSF1B. In some embodiments, the CRISPR gene-editing system for the treatment of DDD comprises one or more sgRNAs targeting exon 10 of eTNFRSF1B. In some embodiments, the CRISPR gene-editing system for the treatment of DDD comprises one or more sgRNAs targeting exon 11 of eTNFRSF1B.

In some embodiments, the CRISPR gene-editing system for the treatment of DDD comprises one or more sgRNAs targeting exon 1 of fTNFRSF1B. In some embodiments, the CRISPR gene-editing system for the treatment of DDD comprises one or more sgRNAs targeting exon 2 of fTNFRSF1B. In some embodiments, the CRISPR gene-editing system for the treatment of DDD comprises one or more sgRNAs targeting exon 3 of fTNFRSF1B. In some embodiments, the CRISPR gene-editing system for the treatment of DDD comprises one or more sgRNAs targeting exon 4 of fTNFRSF1B. In some embodiments, the CRISPR gene-editing system for the treatment of DDD comprises one or more sgRNAs targeting exon 5 of fTNFRSF1B. In some embodiments, the CRISPR gene-editing system for the treatment of DDD comprises one or more sgRNAs targeting exon 6 of fTNFRSF1B. In some embodiments, the CRISPR gene-editing system for the treatment of DDD comprises one or more sgRNAs targeting exon 7 of fTNFRSF1B. In some embodiments, the CRISPR gene-editing system for the treatment of DDD comprises one or more sgRNAs targeting exon 8 of fTNFRSF1B. In some embodiments, the CRISPR gene-editing system for the treatment of DDD comprises one or more sgRNAs targeting exon 9 of fTNFRSF1B. In some embodiments, the CRISPR gene-editing system for the treatment of DDD comprises one or more sgRNAs targeting exon 10 of fTNFRSF1B.

In some embodiments, the CRISPR gene-editing system for the treatment of DDD comprises one or more sgRNAs targeting TNFRSF3. In some embodiments, the CRISPR gene-editing system for the treatment of DDD comprises one or more sgRNAs targeting hTNFRSF3. In some embodiments, the CRISPR gene-editing system for the treatment of DDD comprises one or more sgRNAs targeting cTNFRSF3. In some embodiments, the CRISPR gene-editing system for the treatment of DDD comprises one or more sgRNAs targeting eTNFRSF3. In some embodiments, the CRISPR gene-editing system for the treatment of DDD comprises one or more sgRNAs targeting fTNFRSF3.

In some embodiments, the CRISPR gene-editing system for the treatment of DDD comprises one or more sgRNAs targeting exon 1 of hTNFRSF3. In some embodiments, the CRISPR gene-editing system for the treatment of DDD comprises one or more sgRNAs targeting exon 2 of hTNFRSF3. In some embodiments, the CRISPR gene-editing system for the treatment of DDD comprises one or more sgRNAs targeting exon 3 of hTNFRSF3. In some embodiments, the CRISPR gene-editing system for the treatment of DDD comprises one or more sgRNAs targeting exon 4 of hTNFRSF3. In some embodiments, the CRISPR gene-editing system for the treatment of DDD comprises one or more sgRNAs targeting exon 5 of hTNFRSF3. In some embodiments, the CRISPR gene-editing system for the treatment of DDD comprises one or more sgRNAs targeting exon 6 of hTNFRSF3. In some embodiments, the CRISPR gene-editing system for the treatment of DDD comprises one or more sgRNAs targeting exon 7 of hTNFRSF3. In some embodiments, the CRISPR gene-editing system for the treatment of DDD comprises one or more sgRNAs targeting exon 8 of hTNFRSF3. In some embodiments, the CRISPR gene-editing system for the treatment of DDD comprises one or more sgRNAs targeting exon 9 of hTNFRSF3. In some embodiments, the CRISPR gene-editing system for the treatment of DDD comprises one or more sgRNAs targeting exon 10 of hTNFRSF3. In some embodiments, the CRISPR gene-editing system for the treatment of DDD comprises one or more sgRNAs targeting exon 11 of hTNFRSF3. In some embodiments, the CRISPR gene-editing system for the treatment of DDD comprises one or more sgRNAs targeting exon 12 of hTNFRSF3.

In some embodiments, the CRISPR gene-editing system for the treatment of DDD comprises one or more sgRNAs targeting exon 1 of cTNFRSF3. In some embodiments, the CRISPR gene-editing system for the treatment of DDD comprises one or more sgRNAs targeting exon 2 of cTNFRSF3. In some embodiments, the CRISPR gene-editing system for the treatment of DDD comprises one or more sgRNAs targeting exon 3 of cTNFRSF3. In some embodiments, the CRISPR gene-editing system for the treatment of DDD comprises one or more sgRNAs targeting exon 4 of cTNFRSF3. In some embodiments, the CRISPR gene-editing system for the treatment of DDD comprises one or more sgRNAs targeting exon 5 of cTNFRSF3. In some embodiments, the CRISPR gene-editing system for the treatment of DDD comprises one or more sgRNAs targeting exon 6 of cTNFRSF3. In some embodiments, the CRISPR gene-editing system for the treatment of DDD comprises one or more sgRNAs targeting exon 7 of cTNFRSF3. In some embodiments, the CRISPR gene-editing system for the treatment of DDD comprises one or more sgRNAs targeting exon 8 of cTNFRSF3. In some embodiments, the CRISPR gene-editing system for the treatment of DDD comprises one or more sgRNAs targeting exon 9 of cTNFRSF3. In some embodiments, the CRISPR gene-editing system for the treatment of DDD comprises one or more sgRNAs targeting exon 10 of cTNFRSF3.

In some embodiments, the CRISPR gene-editing system for the treatment of DDD comprises one or more sgRNAs targeting exon 1 of eTNFRSF3. In some embodiments, the CRISPR gene-editing system for the treatment of DDD comprises one or more sgRNAs targeting exon 2 of eTNFRSF3. In some embodiments, the CRISPR gene-editing system for the treatment of DDD comprises one or more sgRNAs targeting exon 3 of eTNFRSF3. In some embodiments, the CRISPR gene-editing system for the treatment of DDD comprises one or more sgRNAs targeting exon 4 of eTNFRSF3. In some embodiments, the CRISPR gene-editing system for the treatment of DDD comprises one or more sgRNAs targeting exon 5 of eTNFRSF3. In some embodiments, the CRISPR gene-editing system for the treatment of DDD comprises one or more sgRNAs targeting exon 6 of eTNFRSF3. In some embodiments, the CRISPR gene-editing system for the treatment of DDD comprises one or more sgRNAs targeting exon 7 of eTNFRSF3. In some embodiments, the CRISPR gene-editing system for the treatment of DDD comprises one or more sgRNAs targeting exon 8 of eTNFRSF3. In some embodiments, the CRISPR gene-editing system for the treatment of DDD comprises one or more sgRNAs targeting exon 9 of eTNFRSF3. In some embodiments, the CRISPR gene-editing system for the treatment of DDD comprises one or more sgRNAs targeting exon 10 of eTNFRSF3.

In some embodiments, the CRISPR gene-editing system for the treatment of DDD comprises one or more sgRNAs targeting exon 1 of fTNFRSF3. In some embodiments, the CRISPR gene-editing system for the treatment of DDD comprises one or more sgRNAs targeting exon 2 of fTNFRSF3. In some embodiments, the CRISPR gene-editing system for the treatment of DDD comprises one or more sgRNAs targeting exon 3 of fTNFRSF3. In some embodiments, the CRISPR gene-editing system for the treatment of DDD comprises one or more sgRNAs targeting exon 4 of fTNFRSF3. In some embodiments, the CRISPR gene-editing system for the treatment of DDD comprises one or more sgRNAs targeting exon 5 of fTNFRSF3. In some embodiments, the CRISPR gene-editing system for the treatment of DDD comprises one or more sgRNAs targeting exon 6 of fTNFRSF3. In some embodiments, the CRISPR gene-editing system for the treatment of DDD comprises one or more sgRNAs targeting exon 7 of fTNFRSF3. In some embodiments, the CRISPR gene-editing system for the treatment of DDD comprises one or more sgRNAs targeting exon 8 of fTNFRSF3. In some embodiments, the CRISPR gene-editing system for the treatment of DDD comprises one or more sgRNAs targeting exon 9 of fTNFRSF3. In some embodiments, the CRISPR gene-editing system for the treatment of DDD comprises one or more sgRNAs targeting exon 10 of fTNFRSF3.

In some embodiments, the CRISPR gene-editing system for the treatment of DDD comprises one or more sgRNAs targeting TNFRSF4. In some embodiments, the CRISPR gene-editing system for the treatment of DDD comprises one or more sgRNAs targeting hTNFRSF4. In some embodiments, the CRISPR gene-editing system for the treatment of DDD comprises one or more sgRNAs targeting cTNFRSF4. In some embodiments, the CRISPR gene-editing system for the treatment of DDD comprises one or more sgRNAs targeting eTNFRSF4. In some embodiments, the CRISPR gene-editing system for the treatment of DDD comprises one or more sgRNAs targeting fTNFRSF4.

In some embodiments, the CRISPR gene-editing system for the treatment of DDD comprises one or more sgRNAs targeting exon 1 of hTNFRSF4. In some embodiments, the CRISPR gene-editing system for the treatment of DDD comprises one or more sgRNAs targeting exon 2 of hTNFRSF4. In some embodiments, the CRISPR gene-editing system for the treatment of DDD comprises one or more sgRNAs targeting exon 3 of hTNFRSF4. In some embodiments, the CRISPR gene-editing system for the treatment of DDD comprises one or more sgRNAs targeting exon 4 of hTNFRSF4. In some embodiments, the CRISPR gene-editing system for the treatment of DDD comprises one or more sgRNAs targeting exon 5 of hTNFRSF4. In some embodiments, the CRISPR gene-editing system for the treatment of DDD comprises one or more sgRNAs targeting exon 6 of hTNFRSF4.

In some embodiments, the CRISPR gene-editing system for the treatment of DDD comprises one or more sgRNAs targeting exon 1 of cTNFRSF4. In some embodiments, the CRISPR gene-editing system for the treatment of DDD comprises one or more sgRNAs targeting exon 2 of cTNFRSF4. In some embodiments, the CRISPR gene-editing system for the treatment of DDD comprises one or more sgRNAs targeting exon 3 of cTNFRSF4. In some embodiments, the CRISPR gene-editing system for the treatment of DDD comprises one or more sgRNAs targeting exon 4 of cTNFRSF4. In some embodiments, the CRISPR gene-editing system for the treatment of DDD comprises one or more sgRNAs targeting exon 5 of cTNFRSF4. In some embodiments, the CRISPR gene-editing system for the treatment of DDD comprises one or more sgRNAs targeting exon 6 of cTNFRSF4. In some embodiments, the CRISPR gene-editing system for the treatment of DDD comprises one or more sgRNAs targeting exon 7 of cTNFRSF4.

In some embodiments, the CRISPR gene-editing system for the treatment of DDD comprises one or more sgRNAs targeting exon 1 of eTNFRSF4. In some embodiments, the CRISPR gene-editing system for the treatment of DDD comprises one or more sgRNAs targeting exon 2 of eTNFRSF4. In some embodiments, the CRISPR gene-editing system for the treatment of DDD comprises one or more sgRNAs targeting exon 3 of eTNFRSF4. In some embodiments, the CRISPR gene-editing system for the treatment of DDD comprises one or more sgRNAs targeting exon 4 of eTNFRSF4. In some embodiments, the CRISPR gene-editing system for the treatment of DDD comprises one or more sgRNAs targeting exon 5 of eTNFRSF4. In some embodiments, the CRISPR gene-editing system for the treatment of DDD comprises one or more sgRNAs targeting exon 6 of eTNFRSF4. In some embodiments, the CRISPR gene-editing system for the treatment of DDD comprises one or more sgRNAs targeting exon 7 of eTNFRSF4.

In some embodiments, the CRISPR gene-editing system for the treatment of DDD comprises one or more sgRNAs targeting exon 1 of fTNFRSF4. In some embodiments, the CRISPR gene-editing system for the treatment of DDD comprises one or more sgRNAs targeting exon 2 of fTNFRSF4. In some embodiments, the CRISPR gene-editing system for the treatment of DDD comprises one or more sgRNAs targeting exon 3 of fTNFRSF4. In some embodiments, the CRISPR gene-editing system for the treatment of DDD comprises one or more sgRNAs targeting exon 4 of fTNFRSF4. In some embodiments, the CRISPR gene-editing system for the treatment of DDD comprises one or more sgRNAs targeting exon 5 of fTNFRSF4. In some embodiments, the CRISPR gene-editing system for the treatment of DDD comprises one or more sgRNAs targeting exon 6 of fTNFRSF4. In some embodiments, the CRISPR gene-editing system for the treatment of DDD comprises one or more sgRNAs targeting exon 7 of fTNFRSF4.

In some embodiments, the CRISPR gene-editing system for the treatment of DDD comprises one or more sgRNAs targeting TNFRSF11A. In some embodiments, the CRISPR gene-editing system for the treatment of DDD comprises one or more sgRNAs targeting hTNFRSF11A. In some embodiments, the CRISPR gene-editing system for the treatment of DDD comprises one or more sgRNAs targeting cTNFRSF11A. In some embodiments, the CRISPR gene-editing system for the treatment of DDD comprises one or more sgRNAs targeting eTNFRSF11A. In some embodiments, the CRISPR gene-editing system for the treatment of DDD comprises one or more sgRNAs targeting fTNFRSF11A.

In some embodiments, the CRISPR gene-editing system for the treatment of DDD comprises one or more sgRNAs targeting exon 1 of hTNFRSF11A. In some embodiments, the CRISPR gene-editing system for the treatment of DDD comprises one or more sgRNAs targeting exon 2 of hTNFRSF11A. In some embodiments, the CRISPR gene-editing system for the treatment of DDD comprises one or more sgRNAs targeting exon 3 of hTNFRSF11A. In some embodiments, the CRISPR gene-editing system for the treatment of DDD comprises one or more sgRNAs targeting exon 4 of hTNFRSF11A. In some embodiments, the CRISPR gene-editing system for the treatment of DDD comprises one or more sgRNAs targeting exon 5 of hTNFRSF11A. In some embodiments, the CRISPR gene-editing system for the treatment of DDD comprises one or more sgRNAs targeting exon 6 of hTNFRSF11A. In some embodiments, the CRISPR gene-editing system for the treatment of DDD comprises one or more sgRNAs targeting exon 7 of hTNFRSF11A. In some embodiments, the CRISPR gene-editing system for the treatment of DDD comprises one or more sgRNAs targeting exon 8 of hTNFRSF11A. In some embodiments, the CRISPR gene-editing system for the treatment of DDD comprises one or more sgRNAs targeting exon 9 of hTNFRSF11A. In some embodiments, the CRISPR gene-editing system for the treatment of DDD comprises one or more sgRNAs targeting exon 10 of hTNFRSF11A. In some embodiments, the CRISPR gene-editing system for the treatment of DDD comprises one or more sgRNAs targeting exon 11 of hTNFRSF11A. In some embodiments, the CRISPR gene-editing system for the treatment of DDD comprises one or more sgRNAs targeting exon 12 of hTNFRSF11A.

In some embodiments, the CRISPR gene-editing system for the treatment of DDD comprises one or more sgRNAs targeting exon 1 of cTNFRSF11A. In some embodiments, the CRISPR gene-editing system for the treatment of DDD comprises one or more sgRNAs targeting exon 2 of cTNFRSF11A. In some embodiments, the CRISPR gene-editing system for the treatment of DDD comprises one or more sgRNAs targeting exon 3 of cTNFRSF11A. In some embodiments, the CRISPR gene-editing system for the treatment of DDD comprises one or more sgRNAs targeting exon 4 of cTNFRSF11A. In some embodiments, the CRISPR gene-editing system for the treatment of DDD comprises one or more sgRNAs targeting exon 5 of cTNFRSF11A. In some embodiments, the CRISPR gene-editing system for the treatment of DDD comprises one or more sgRNAs targeting exon 6 of cTNFRSF11A. In some embodiments, the CRISPR gene-editing system for the treatment of DDD comprises one or more sgRNAs targeting exon 7 of cTNFRSF11A. In some embodiments, the CRISPR gene-editing system for the treatment of DDD comprises one or more sgRNAs targeting exon 8 of cTNFRSF11A. In some embodiments, the CRISPR gene-editing system for the treatment of DDD comprises one or more sgRNAs targeting exon 9 of cTNFRSF11A. In some embodiments, the CRISPR gene-editing system for the treatment of DDD comprises one or more sgRNAs targeting exon 10 of cTNFRSF11A. In some embodiments, the CRISPR gene-editing system for the treatment of DDD comprises one or more sgRNAs targeting exon 11 of cTNFRSF11A.

In some embodiments, the CRISPR gene-editing system for the treatment of DDD comprises one or more sgRNAs targeting exon 1 of eTNFRSF11A. In some embodiments, the CRISPR gene-editing system for the treatment of DDD comprises one or more sgRNAs targeting exon 2 of eTNFRSF11A. In some embodiments, the CRISPR gene-editing system for the treatment of DDD comprises one or more sgRNAs targeting exon 3 of eTNFRSF11A. In some embodiments, the CRISPR gene-editing system for the treatment of DDD comprises one or more sgRNAs targeting exon 4 of eTNFRSF11A. In some embodiments, the CRISPR gene-editing system for the treatment of DDD comprises one or more sgRNAs targeting exon 5 of eTNFRSF11A. In some embodiments, the CRISPR gene-editing system for the treatment of DDD comprises one or more sgRNAs targeting exon 6 of eTNFRSF11A. In some embodiments, the CRISPR gene-editing system for the treatment of DDD comprises one or more sgRNAs targeting exon 7 of eTNFRSF11A. In some embodiments, the CRISPR gene-editing system for the treatment of DDD comprises one or more sgRNAs targeting exon 8 of eTNFRSF11A. In some embodiments, the CRISPR gene-editing system for the treatment of DDD comprises one or more sgRNAs targeting exon 9 of eTNFRSF11A. In some embodiments, the CRISPR gene-editing system for the treatment of DDD comprises one or more sgRNAs targeting exon 10 of eTNFRSF11A.

In some embodiments, the CRISPR gene-editing system for the treatment of DDD comprises one or more sgRNAs targeting exon 1 of fTNFRSF11A. In some embodiments, the CRISPR gene-editing system for the treatment of DDD comprises one or more sgRNAs targeting exon 2 of fTNFRSF11A. In some embodiments, the CRISPR gene-editing system for the treatment of DDD comprises one or more sgRNAs targeting exon 3 of fTNFRSF11A. In some embodiments, the CRISPR gene-editing system for the treatment of DDD comprises one or more sgRNAs targeting exon 4 of fTNFRSF11A. In some embodiments, the CRISPR gene-editing system for the treatment of DDD comprises one or more sgRNAs targeting exon 5 of fTNFRSF11A. In some embodiments, the CRISPR gene-editing system for the treatment of DDD comprises one or more sgRNAs targeting exon 6 of fTNFRSF11A. In some embodiments, the CRISPR gene-editing system for the treatment of DDD comprises one or more sgRNAs targeting exon 7 of fTNFRSF11A. In some embodiments, the CRISPR gene-editing system for the treatment of DDD comprises one or more sgRNAs targeting exon 8 of fTNFRSF11A. In some embodiments, the CRISPR gene-editing system for the treatment of DDD comprises one or more sgRNAs targeting exon 9 of fTNFRSF11A. In some embodiments, the CRISPR gene-editing system for the treatment of DDD comprises one or more sgRNAs targeting exon 10 of fTNFRSF11A. In some embodiments, the CRISPR gene-editing system for the treatment of DDD comprises one or more sgRNAs targeting exon 11 of fTNFRSF11A.

### D. IDD

In one aspect, the present disclosure encompasses treatments for internal disc disruption (IDD). In some embodiments, the IDD treatment comprises a therapeutically effective amount of a Clustered Regularly Interspaced Short Palindromic Repeats (CRISPR) gene-editing system, the system comprising: (i) a CRISPR Associated (Cas) protein; and (ii) at least one guide RNA targeting IL1R1. In some embodiments, the IDD treatment comprises a CRISPR gene-editing system targeting hIL1R1. In some embodiments, the IDD treatment comprises a CRISPR gene-editing system targeting cIL1R1. In some embodiments, the IDD treatment comprises a CRISP gene-editing system R targeting eIL1R1. In some embodiments, the IDD treatment comprises a CRISPR gene-editing system targeting fIL1R1.

In some embodiments, the CRISPR gene-editing system for the treatment of IDD comprises one or more sgRNAs targeting exon 1 of hIL1R1. In some embodiments, the CRISPR gene-editing system for the treatment of IDD comprises one or more sgRNAs targeting exon 2 of hIL1R1. In some embodiments, the CRISPR gene-editing system for the treatment of IDD comprises one or more sgRNAs targeting exon 3 of hIL1R1. In some embodiments, the CRISPR gene-editing system for the treatment of IDD comprises one or more sgRNAs targeting exon 4 of hIL1R1. In some embodiments, the CRISPR gene-editing system for the treatment of IDD comprises one or more sgRNAs targeting exon 5 of hIL1R1. In some embodiments, the CRISPR gene-editing system for the treatment of IDD comprises one or more sgRNAs targeting exon 6 of hIL1R1. In some embodiments, the CRISPR gene-editing system for the treatment of IDD comprises one or more sgRNAs targeting exon 7 of hIL1R1. In some embodiments, the CRISPR gene-editing system for the treatment of IDD comprises one or more sgRNAs targeting exon 8 of hIL1R1. In some embodiments, the CRISPR gene-editing system for the treatment of IDD comprises one or more sgRNAs targeting exon 9 of hIL1R1. In some embodiments, the CRISPR gene-editing system for the treatment of IDD comprises one or more sgRNAs targeting exon 10 of hIL1R1. In some embodiments, the CRISPR gene-editing system for the treatment of IDD comprises one or more sgRNAs targeting exon 11 of hIL1R1. In some embodiments, the CRISPR gene-editing system for the treatment of IDD comprises one or more sgRNAs targeting exon 12 of hIL1R1. In some embodiments, the CRISPR gene-editing system for the treatment of IDD comprises one or more sgRNAs targeting exon 13 of hIL1R1. In some embodiments, the CRISPR gene-editing system for the treatment of IDD comprises one or more sgRNAs targeting exon 14 of hIL1R1. In some embodiments, the CRISPR gene-editing system for the treatment of IDD comprises one or more sgRNAs targeting exon 15 of hIL1R1. In some embodiments, the CRISPR gene-editing system for the treatment of IDD comprises one or more sgRNAs targeting exon 16 of hIL1R1. In some embodiments, the CRISPR gene-editing system for the treatment of IDD comprises one or more sgRNAs targeting exon 17 of hIL1R1. In some embodiments, the CRISPR gene-editing system for the treatment of IDD comprises one or more sgRNAs targeting exon 18 of hIL1R1. In some embodiments, the CRISPR gene-editing system for the treatment of IDD comprises one or more sgRNAs targeting exon 19 of hIL1R1. In some embodiments, the CRISPR gene-editing system for the treatment of IDD comprises one or more sgRNAs targeting exon 20 of hIL1R1. In some embodiments, the CRISPR gene-editing system for the treatment of IDD comprises one or more sgRNAs targeting exon 21 of hIL1R1.

In some embodiments, the CRISPR gene-editing system for the treatment of IDD comprises one or more sgRNAs targeting exon 1 of cIL1R1. In some embodiments, the CRISPR gene-editing system for the treatment of IDD comprises one or more sgRNAs targeting exon 2 of cIL1R1. In some embodiments, the CRISPR gene-editing system for the treatment of IDD comprises one or more sgRNAs targeting exon 3 of cIL1R1. In some embodiments, the CRISPR gene-editing system for the treatment of IDD comprises one or more sgRNAs targeting exon 4 of cIL1R1. In some embodiments, the CRISPR gene-editing system for the treatment of IDD comprises one or more sgRNAs targeting exon 5 of cIL1R1. In some embodiments, the CRISPR gene-editing system for the treatment of IDD comprises one or more sgRNAs targeting exon 6 of cIL1R1. In some embodiments, the CRISPR gene-editing system for the treatment of IDD comprises one or more sgRNAs targeting exon 7 of cIL1R1. In some embodiments, the CRISPR gene-editing system for the treatment of IDD comprises one or more sgRNAs targeting exon 8 of cIL1R1. In some embodiments, the CRISPR gene-editing system for the treatment of IDD comprises one or more sgRNAs targeting exon 9 of cIL1R1. In some embodiments, the CRISPR gene-editing system for the treatment of IDD comprises one or more sgRNAs targeting exon 10 of cIL1R1. In some embodiments, the CRISPR gene-editing system for the treatment of IDD comprises one or more sgRNAs targeting exon 11 of cIL1R1. In some embodiments, the CRISPR gene-editing system for the treatment of IDD comprises one or more sgRNAs targeting exon 12 of cIL1R1. In some embodiments, the CRISPR gene-editing system for the treatment of IDD comprises one or more sgRNAs targeting exon 13 of cIL1R1. In some embodiments, the CRISPR gene-editing system for the treatment of IDD comprises one or more sgRNAs targeting exon 14 of cIL1R1. In some embodiments, the CRISPR gene-editing system for the treatment of IDD comprises one or more sgRNAs targeting exon 15 of cIL1R1. In some embodiments, the CRISPR gene-editing system for the treatment of IDD comprises one or more sgRNAs targeting exon 16 of cIL1R1.

In some embodiments, the CRISPR gene-editing system for the treatment of IDD comprises one or more sgRNAs targeting exon 1 of eIL1R1. In some embodiments, the CRISPR gene-editing system for the treatment of IDD comprises one or more sgRNAs targeting exon 2 of eIL1R1. In some embodiments, the CRISPR gene-editing system for the treatment of IDD comprises one or more sgRNAs targeting exon 3 of eIL1R1. In some embodiments, the CRISPR gene-editing system for the treatment of IDD comprises one or more sgRNAs targeting exon 4 of eIL1R1. In some embodiments, the CRISPR gene-editing system for the treatment of IDD comprises one or more sgRNAs targeting exon 5 of eIL1R1. In some embodiments, the CRISPR gene-editing system for the treatment of IDD comprises one or more sgRNAs targeting exon 6 of eIL1R1. In some embodiments, the CRISPR gene-editing system for the treatment of IDD comprises one or more sgRNAs targeting exon 7 of eIL1R1. In some embodiments, the CRISPR gene-editing system for the treatment of IDD comprises one or more sgRNAs targeting exon 8 of eIL1R1. In some embodiments, the CRISPR gene-editing system for the treatment of IDD comprises one or more sgRNAs targeting exon 9 of eIL1R1. In some embodiments, the CRISPR gene-editing system for the treatment of IDD comprises one or more sgRNAs targeting exon 10 of eIL1R1. In some embodiments, the CRISPR gene-editing system for the treatment of IDD comprises one or more sgRNAs targeting exon 11 of eIL1R1. In some embodiments, the CRISPR gene-editing system for the treatment of IDD comprises one or more sgRNAs targeting exon 12 of eIL1R1. In some embodiments, the CRISPR gene-editing system for the treatment of IDD comprises one or more sgRNAs targeting exon 13 of eIL1R1. In some embodiments, the CRISPR gene-editing system for the treatment of IDD comprises one or more sgRNAs targeting exon 14 of eIL1R1. In some embodiments, the CRISPR gene-editing system for the treatment of IDD comprises one or more sgRNAs targeting exon 15 of eIL1R1.

In some embodiments, the CRISPR gene-editing system for the treatment of IDD comprises one or more sgRNAs targeting exon 1 of fIL1R1. In some embodiments, the CRISPR gene-editing system for the treatment of IDD comprises one or more sgRNAs targeting exon 2 of fIL1R1. In some embodiments, the CRISPR gene-editing system for the treatment of IDD comprises one or more sgRNAs targeting exon 3 of fIL1R1. In some embodiments, the CRISPR gene-editing system for the treatment of IDD comprises one or more sgRNAs targeting exon 4 of fIL1R1. In some embodiments, the CRISPR gene-editing system for the treatment of IDD comprises one or more sgRNAs targeting exon 5 of fIL1R1. In some embodiments, the CRISPR gene-editing system for the treatment of IDD comprises one or more sgRNAs targeting exon 6 of fIL1R1. In some embodiments, the CRISPR gene-editing system for the treatment of IDD comprises one or more sgRNAs targeting exon 7 of fIL1R1. In some embodiments, the CRISPR gene-editing system for the treatment of IDD comprises one or more sgRNAs targeting exon 8 of fIL1R1. In some embodiments, the CRISPR gene-editing system for the treatment of IDD comprises one or more sgRNAs targeting exon 9 of fIL1R1. In some embodiments, the CRISPR gene-editing system for the treatment of IDD comprises one or more sgRNAs targeting exon 10 of fIL1R1. In some embodiments, the CRISPR gene-editing system for the treatment of IDD comprises one or more sgRNAs targeting exon 11 of fIL1R1. In some embodiments, the CRISPR gene-editing system for the treatment of IDD comprises one or more sgRNAs targeting exon 12 of fIL1R1. In some embodiments, the CRISPR gene-editing system for the treatment of IDD comprises one or more sgRNAs targeting exon 13 of fIL1R1. In some embodiments, the CRISPR gene-editing system for the treatment of IDD comprises one or more sgRNAs targeting exon 14 of fIL1R1. In some embodiments, the CRISPR gene-editing system for the treatment of IDD comprises one or more sgRNAs targeting exon 15 of fIL1R1. In some embodiments, the CRISPR gene-editing system for the treatment of IDD comprises one or more sgRNAs targeting exon 16 of fIL1R1. In some embodiments, the CRISPR gene-editing system for the treatment of IDD comprises one or more sgRNAs targeting exon 17 of fIL1R1. In some embodiments, the CRISPR gene-editing system for the treatment of IDD comprises one or more sgRNAs targeting exon 18 of fIL1R1.

In some embodiments, the CRISPR gene-editing system for the treatment of IDD comprises one or more sgRNAs targeting IL1RAP. In some embodiments, the CRISPR gene-editing system for the treatment of IDD comprises one or more sgRNAs targeting hIL1RAP. In some embodiments, the CRISPR gene-editing system for the treatment of IDD comprises one or more sgRNAs targeting cIL1RAP. In some embodiments, the CRISPR gene-editing system for the treatment of IDD comprises one or more sgRNAs targeting eIL1RAP. In some embodiments, the CRISPR gene-editing system for the treatment of IDD comprises one or more sgRNAs targeting fIL1RAP.

In some embodiments, the CRISPR gene-editing system for the treatment of IDD comprises one or more sgRNAs targeting exon 1 of hIL1RAP. In some embodiments, the CRISPR gene-editing system for the treatment of IDD comprises one or more sgRNAs targeting exon 2 of hIL1RAP. In some embodiments, the CRISPR gene-editing system for the treatment of IDD comprises one or more sgRNAs targeting exon 3 of hIL1RAP. In some embodiments, the CRISPR gene-editing system for the treatment of IDD comprises one or more sgRNAs targeting exon 4 of hIL1RAP. In some embodiments, the CRISPR gene-editing system for the treatment of IDD comprises one or more sgRNAs targeting exon 5 of hIL1RAP. In some embodiments, the CRISPR gene-editing system for the treatment of IDD comprises one or more sgRNAs targeting exon 6 of hIL1RAP. In some embodiments, the CRISPR gene-editing system for the treatment of IDD comprises one or more sgRNAs targeting exon 7 of hIL1RAP. In some embodiments, the CRISPR gene-editing system for the treatment of IDD comprises one or more sgRNAs targeting exon 8 of hIL1RAP. In some embodiments, the CRISPR gene-editing system for the treatment of IDD comprises one or more sgRNAs targeting exon 9 of hIL1RAP. In some embodiments, the CRISPR gene-editing system for the treatment of IDD comprises one or more sgRNAs targeting exon 10 of hIL1RAP. In some embodiments, the CRISPR gene-editing system for the treatment of IDD comprises one or more sgRNAs targeting exon 11 of hIL1RAP. In some embodiments, the CRISPR gene-editing system for the treatment of IDD comprises one or more sgRNAs targeting exon 12 of hIL1RAP. In some embodiments, the CRISPR gene-editing system for the treatment of IDD comprises one or more sgRNAs targeting exon 13 of hIL1RAP. In some embodiments, the CRISPR gene-editing system for the treatment of IDD comprises one or more sgRNAs targeting exon 14 of hIL1RAP. In some embodiments, the CRISPR gene-editing system for the treatment of IDD comprises one or more sgRNAs targeting exon 15 of hIL1RAP. In some embodiments, the CRISPR gene-editing system for the treatment of IDD comprises one or more sgRNAs targeting exon 16 of hIL1RAP. In some embodiments, the CRISPR gene-editing system for the treatment of IDD comprises one or more sgRNAs targeting exon 17 of hIL1RAP.

In some embodiments, the CRISPR gene-editing system for the treatment of IDD comprises one or more sgRNAs targeting exon 1 of cIL1RAP. In some embodiments, the CRISPR gene-editing system for the treatment of IDD comprises one or more sgRNAs targeting exon 2 of cIL1RAP. In some embodiments, the CRISPR gene-editing system for the treatment of IDD comprises one or more sgRNAs targeting exon 3 of cIL1RAP. In some embodiments, the CRISPR gene-editing system for the treatment of IDD comprises one or more sgRNAs targeting exon 4 of cIL1RAP. In some embodiments, the CRISPR gene-editing system for the treatment of IDD comprises one or more sgRNAs targeting exon 5 of cIL1RAP. In some embodiments, the CRISPR gene-editing system for the treatment of IDD comprises one or more sgRNAs targeting exon 6 of cIL1RAP. In some embodiments, the CRISPR gene-editing system for the treatment of IDD comprises one or more sgRNAs targeting exon 7 of cIL1RAP. In some embodiments, the CRISPR gene-editing system for the treatment of IDD comprises one or more sgRNAs targeting exon 8 of cIL1RAP. In some embodiments, the CRISPR gene-editing system for the treatment of IDD comprises one or more sgRNAs targeting exon 9 of cIL1RAP. In some embodiments, the CRISPR gene-editing system for the treatment of IDD comprises one or more sgRNAs targeting exon 10 of cIL1RAP. In some embodiments, the CRISPR gene-editing system for the treatment of IDD comprises one or more sgRNAs targeting exon 11 of cIL1RAP. In some embodiments, the CRISPR gene-editing system for the treatment of IDD comprises one or more sgRNAs targeting exon 12 of cIL1RAP. In some embodiments, the CRISPR gene-editing system for the treatment of IDD comprises one or more sgRNAs targeting exon 13 of cIL1RAP. In some embodiments, the CRISPR gene-editing system for the treatment of IDD comprises one or more sgRNAs targeting exon 14 of cIL1RAP. In some embodiments, the CRISPR gene-editing system for the treatment of IDD comprises one or more sgRNAs targeting exon 15 of cIL1RAP. In some embodiments, the CRISPR gene-editing system for the treatment of IDD comprises one or more sgRNAs targeting exon 16 of cIL1RAP.

In some embodiments, the CRISPR gene-editing system for the treatment of IDD comprises one or more sgRNAs targeting exon 1 of eIL1RAP. In some embodiments, the CRISPR gene-editing system for the treatment of IDD comprises one or more sgRNAs targeting exon 2 of eIL1RAP. In some embodiments, the CRISPR gene-editing system for the treatment of IDD comprises one or more sgRNAs targeting exon 3 of eIL1RAP. In some embodiments, the CRISPR gene-editing system for the treatment of IDD comprises one or more sgRNAs targeting exon 4 of eIL1RAP. In some embodiments, the CRISPR gene-editing system for the treatment of IDD comprises one or more sgRNAs targeting exon 5 of eIL1RAP. In some embodiments, the CRISPR gene-editing system for the treatment of IDD comprises one or more sgRNAs targeting exon 6 of eIL1RAP. In some embodiments, the CRISPR gene-editing system for the treatment of IDD comprises one or more sgRNAs targeting exon 7 of eIL1RAP. In some embodiments, the CRISPR gene-editing system for the treatment of IDD comprises one or more sgRNAs targeting exon 8 of eIL1RAP. In some embodiments, the CRISPR gene-editing system for the treatment of IDD comprises one or more sgRNAs targeting exon 9 of eIL1RAP. In some embodiments, the CRISPR gene-editing system for the treatment of IDD comprises one or more sgRNAs targeting exon 10 of eIL1RAP. In some embodiments, the CRISPR gene-editing system for the treatment of IDD comprises one or more sgRNAs targeting exon 11 of eIL1RAP. In some embodiments, the CRISPR gene-editing system for the treatment of IDD comprises one or more sgRNAs targeting exon 12 of eIL1RAP. In some embodiments, the CRISPR gene-editing system for the treatment of IDD comprises one or more sgRNAs targeting exon 13 of eIL1RAP.

In some embodiments, the CRISPR gene-editing system for the treatment of IDD comprises one or more sgRNAs targeting exon 1 of fIL1RAP. In some embodiments, the CRISPR gene-editing system for the treatment of IDD comprises one or more sgRNAs targeting exon 2 of fIL1RAP. In some embodiments, the CRISPR gene-editing system for the treatment of IDD comprises one or more sgRNAs targeting exon 3 of fIL1RAP. In some embodiments, the CRISPR gene-editing system for the treatment of IDD comprises one or more sgRNAs targeting exon 4 of fIL1RAP. In some embodiments, the CRISPR gene-editing system for the treatment of IDD comprises one or more sgRNAs targeting exon 5 of fIL1RAP. In some embodiments, the CRISPR gene-editing system for the treatment of IDD comprises one or more sgRNAs targeting exon 6 of fIL1RAP. In some embodiments, the CRISPR gene-editing system for the treatment of IDD comprises one or more sgRNAs targeting exon 7 of fIL1RAP. In some embodiments, the CRISPR gene-editing system for the treatment of IDD comprises one or more sgRNAs targeting exon 8 of fIL1RAP. In some embodiments, the CRISPR gene-editing system for the treatment of IDD comprises one or more sgRNAs targeting exon 9 of fIL1RAP. In some embodiments, the CRISPR gene-editing system for the treatment of IDD comprises one or more sgRNAs targeting exon 10 of fIL1RAP. In some embodiments, the CRISPR gene-editing system for the treatment of IDD comprises one or more sgRNAs targeting exon 11 of fIL1RAP. In some embodiments, the CRISPR gene-editing system for the treatment of IDD comprises one or more sgRNAs targeting exon 12 of fIL1RAP. In some embodiments, the CRISPR gene-editing system for the treatment of IDD comprises one or more sgRNAs targeting exon 13 of fIL1RAP. In some embodiments, the CRISPR gene-editing system for the treatment of IDD comprises one or more sgRNAs targeting exon 14 of fIL1RAP.

In some embodiments, the CRISPR gene-editing system for the treatment of IDD comprises one or more sgRNAs targeting TGFBR1. In some embodiments, the CRISPR gene-editing system for the treatment of IDD comprises one or more sgRNAs targeting hTGFBR1. In some embodiments, the CRISPR gene-editing system for the treatment of IDD comprises one or more sgRNAs targeting cTGFBR1. In some embodiments, the CRISPR gene-editing system for the treatment of IDD comprises one or more sgRNAs targeting e TGFBR1. In some embodiments, the CRISPR gene-editing system for the treatment of IDD comprises one or more sgRNAs targeting fTGFBR1.

In some embodiments, the CRISPR gene-editing system for the treatment of IDD comprises one or more sgRNAs targeting exon 1 of hTGFBR1. In some embodiments, the CRISPR gene-editing system for the treatment of IDD comprises one or more sgRNAs targeting exon 2 of hTGFBR1. In some embodiments, the CRISPR gene-editing system for the treatment of IDD comprises one or more sgRNAs targeting exon 3 of hTGFBR1. In some embodiments, the CRISPR gene-editing system for the treatment of IDD comprises one or more sgRNAs targeting exon 4 of hTGFBR1. In some embodiments, the CRISPR gene-editing system for the treatment of IDD comprises one or more sgRNAs targeting exon 5 of hTGFBR1. In some embodiments, the CRISPR gene-editing system for the treatment of IDD comprises one or more sgRNAs targeting exon 6 of hTGFBR1. In some embodiments, the CRISPR gene-editing system for the treatment of IDD comprises one or more sgRNAs targeting exon 7 of hTGFBR1. In some embodiments, the CRISPR gene-editing system for the treatment of IDD comprises one or more sgRNAs targeting exon 8 of hTGFBR1. In some embodiments, the CRISPR gene-editing system for the treatment of IDD comprises one or more sgRNAs targeting exon 9 of hTGFBR1. In some embodiments, the CRISPR gene-editing system for the treatment of IDD comprises one or more sgRNAs targeting exon 10 of hTGFBR1. In some embodiments, the CRISPR gene-editing system for the treatment of IDD comprises one or more sgRNAs targeting exon 11 of hTGFBR1.

In some embodiments, the CRISPR gene-editing system for the treatment of IDD comprises one or more sgRNAs targeting exon 1 of cTGFBR1. In some embodiments, the CRISPR gene-editing system for the treatment of IDD comprises one or more sgRNAs targeting exon 2 of cTGFBR1. In some embodiments, the CRISPR gene-editing system for the treatment of IDD comprises one or more sgRNAs targeting exon 3 of cTGFBR1. In some embodiments, the CRISPR gene-editing system for the treatment of IDD comprises one or more sgRNAs targeting exon 4 of cTGFBR1. In some embodiments, the CRISPR gene-editing system for the treatment of IDD comprises one or more sgRNAs targeting exon 5 of cTGFBR1. In some embodiments, the CRISPR gene-editing system for the treatment of IDD comprises one or more sgRNAs targeting exon 6 of cTGFBR1. In some embodiments, the CRISPR gene-editing system for the treatment of IDD comprises one or more sgRNAs targeting exon 7 of cTGFBR1. In some embodiments, the CRISPR gene-editing system for the treatment of IDD comprises one or more sgRNAs targeting exon 8 of cTGFBR1. In some embodiments, the CRISPR gene-editing system for the treatment of IDD comprises one or more sgRNAs targeting exon 9 of cTGFBR1.

In some embodiments, the CRISPR gene-editing system for the treatment of IDD comprises one or more sgRNAs targeting exon 1 of eTGFBR1. In some embodiments, the CRISPR gene-editing system for the treatment of IDD comprises one or more sgRNAs targeting exon 2 of eTGFBR1. In some embodiments, the CRISPR gene-editing system for the treatment of IDD comprises one or more sgRNAs targeting exon 3 of eTGFBR1. In some embodiments, the CRISPR gene-editing system for the treatment of IDD comprises one or more sgRNAs targeting exon 4 of eTGFBR1. In some embodiments, the CRISPR gene-editing system for the treatment of IDD comprises one or more sgRNAs targeting exon 5 of eTGFBR1. In some embodiments, the CRISPR gene-editing system for the treatment of IDD comprises one or more sgRNAs targeting exon 6 of eTGFBR1. In some embodiments, the CRISPR gene-editing system for the treatment of IDD comprises one or more sgRNAs targeting exon 7 of eTGFBR1. In some embodiments, the CRISPR gene-editing system for the treatment of IDD comprises one or more sgRNAs targeting exon 8 of eTGFBR1. In some embodiments, the CRISPR gene-editing system for the treatment of IDD comprises one or more sgRNAs targeting exon 9 of eTGFBR1. In some embodiments, the CRISPR gene-editing system for the treatment of IDD comprises one or more sgRNAs targeting exon 10 of eTGFBR1. In some embodiments, the CRISPR gene-editing system for the treatment of IDD comprises one or more sgRNAs targeting exon 11 of eTGFBR1. In some embodiments, the CRISPR gene-editing system for the treatment of IDD comprises one or more sgRNAs targeting exon 12 of eTGFBR1.

In some embodiments, the CRISPR gene-editing system for the treatment of IDD comprises one or more sgRNAs targeting exon 1 of fTGFBR1. In some embodiments, the CRISPR gene-editing system for the treatment of IDD comprises one or more sgRNAs targeting exon 2 of fTGFBR1. In some embodiments, the CRISPR gene-editing system for the treatment of IDD comprises one or more sgRNAs targeting exon 3 of fTGFBR1. In some embodiments, the CRISPR gene-editing system for the treatment of IDD comprises one or more sgRNAs targeting exon 4 of fTGFBR1. In some embodiments, the CRISPR gene-editing system for the treatment of IDD comprises one or more sgRNAs targeting exon 5 of fTGFBR1. In some embodiments, the CRISPR gene-editing system for the treatment of IDD comprises one or more sgRNAs targeting exon 6 of fTGFBR1. In some embodiments, the CRISPR gene-editing system for the treatment of IDD comprises one or more sgRNAs targeting exon 7 of fTGFBR1. In some embodiments, the CRISPR gene-editing system for the treatment of IDD comprises one or more sgRNAs targeting exon 8 of fTGFBR1. In some embodiments, the CRISPR gene-editing system for the treatment of IDD comprises one or more sgRNAs targeting exon 9 of fTGFBR1. In some embodiments, the CRISPR gene-editing system for the treatment of IDD comprises one or more sgRNAs targeting exon 10 of fTGFBR1. In some embodiments, the CRISPR gene-editing system for the treatment of IDD comprises one or more sgRNAs targeting exon 11 of fTGFBR1.

In some embodiments, the CRISPR gene-editing system for the treatment of IDD comprises one or more sgRNAs targeting TGFBR2. In some embodiments, the CRISPR gene-editing system for the treatment of IDD comprises one or more sgRNAs targeting hTGFBR2. In some embodiments, the CRISPR gene-editing system for the treatment of IDD comprises one or more sgRNAs targeting cTGFBR2. In some embodiments, the CRISPR gene-editing system for the treatment of IDD comprises one or more sgRNAs targeting eTGFBR2. In some embodiments, the CRISPR gene-editing system for the treatment of IDD comprises one or more sgRNAs targeting fTGFBR2.

In some embodiments, the CRISPR gene-editing system for the treatment of IDD comprises one or more sgRNAs targeting exon 1 of hTGFBR2. In some embodiments, the CRISPR gene-editing system for the treatment of IDD comprises one or more sgRNAs targeting exon 2 of hTGFBR2. In some embodiments, the CRISPR gene-editing system for the treatment of IDD comprises one or more sgRNAs targeting exon 3 of hTGFBR2. In some embodiments, the CRISPR gene-editing system for the treatment of IDD comprises one or more sgRNAs targeting exon 4 of hTGFBR2. In some embodiments, the CRISPR gene-editing system for the treatment of IDD comprises one or more sgRNAs targeting exon 5 of hTGFBR2. In some embodiments, the CRISPR gene-editing system for the treatment of IDD comprises one or more sgRNAs targeting exon 6 of hTGFBR2. In some embodiments, the CRISPR gene-editing system for the treatment of IDD comprises one or more sgRNAs targeting exon 7 of hTGFBR2. In some embodiments, the CRISPR gene-editing system for the treatment of IDD comprises one or more sgRNAs targeting exon 8 of hTGFBR2. In some embodiments, the CRISPR gene-editing system for the treatment of IDD comprises one or more sgRNAs targeting exon 9 of hTGFBR2.

In some embodiments, the CRISPR gene-editing system for the treatment of IDD comprises one or more sgRNAs targeting exon 1 of cTGFBR2. In some embodiments, the CRISPR gene-editing system for the treatment of IDD comprises one or more sgRNAs targeting exon 2 of cTGFBR2. In some embodiments, the CRISPR gene-editing system for the treatment of IDD comprises one or more sgRNAs targeting exon 3 of cTGFBR2. In some embodiments, the CRISPR gene-editing system for the treatment of IDD comprises one or more sgRNAs targeting exon 4 of cTGFBR2. In some embodiments, the CRISPR gene-editing system for the treatment of IDD comprises one or more sgRNAs targeting exon 5 of cTGFBR2. In some embodiments, the CRISPR gene-editing system for the treatment of IDD comprises one or more sgRNAs targeting exon 6 of cTGFBR2. In some embodiments, the CRISPR gene-editing system for the treatment of IDD comprises one or more sgRNAs targeting exon 7 of cTGFBR2. In some embodiments, the CRISPR gene-editing system for the treatment of IDD comprises one or more sgRNAs targeting exon 8 of cTGFBR2.

In some embodiments, the CRISPR gene-editing system for the treatment of IDD comprises one or more sgRNAs targeting exon 1 of eTGFBR2. In some embodiments, the CRISPR gene-editing system for the treatment of IDD comprises one or more sgRNAs targeting exon 2 of eTGFBR2. In some embodiments, the CRISPR gene-editing system for the treatment of IDD comprises one or more sgRNAs targeting exon 3 of eTGFBR2. In some embodiments, the CRISPR gene-editing system for the treatment of IDD comprises one or more sgRNAs targeting exon 4 of eTGFBR2. In some embodiments, the CRISPR gene-editing system for the treatment of IDD comprises one or more sgRNAs targeting exon 5 of eTGFBR2. In some embodiments, the CRISPR gene-editing system for the treatment of IDD comprises one or more sgRNAs targeting exon 6 of eTGFBR2. In some embodiments, the CRISPR gene-editing system for the treatment of IDD comprises one or more sgRNAs targeting exon 7 of eTGFBR2. In some embodiments, the CRISPR gene-editing system for the treatment of IDD comprises one or more sgRNAs targeting exon 8 of eTGFBR2. In some embodiments, the CRISPR gene-editing system for the treatment of IDD comprises one or more sgRNAs targeting exon 9 of eTGFBR2. In some embodiments, the CRISPR gene-editing system for the treatment of IDD comprises one or more sgRNAs targeting exon 10 of eTGFBR2.

In some embodiments, the CRISPR gene-editing system for the treatment of IDD comprises one or more sgRNAs targeting exon 1 of fTGFBR2. In some embodiments, the CRISPR gene-editing system for the treatment of IDD comprises one or more sgRNAs targeting exon 2 of fTGFBR2. In some embodiments, the CRISPR gene-editing system for the treatment of IDD comprises one or more sgRNAs targeting exon 3 of fTGFBR2. In some embodiments, the CRISPR gene-editing system for the treatment of IDD comprises one or more sgRNAs targeting exon 4 of fTGFBR2. In some embodiments, the CRISPR gene-editing system for the treatment of IDD comprises one or more sgRNAs targeting exon 5 of fTGFBR2. In some embodiments, the CRISPR gene-editing system for the treatment of IDD comprises one or more sgRNAs targeting exon 6 of fTGFBR2. In some embodiments, the CRISPR gene-editing system for the treatment of IDD comprises one or more sgRNAs targeting exon 7 of fTGFBR2. In some embodiments, the CRISPR gene-editing system for the treatment of IDD comprises one or more sgRNAs targeting exon 8 of fTGFBR2.

In some embodiments, the CRISPR gene-editing system for the treatment of IDD comprises one or more sgRNAs targeting IL6R. In some embodiments, the CRISPR gene-editing system for the treatment of IDD comprises one or more sgRNAs targeting hIL6R. In some embodiments, the CRISPR gene-editing system for the treatment of IDD comprises one or more sgRNAs targeting cIL6R. In some embodiments, the CRISPR gene-editing system for the treatment of IDD comprises one or more sgRNAs targeting eIL6R. In some embodiments, the CRISPR gene-editing system for the treatment of IDD comprises one or more sgRNAs targeting fIL6R.

In some embodiments, the CRISPR gene-editing system for the treatment of IDD comprises one or more sgRNAs targeting exon 1 of hIL6R. In some embodiments, the CRISPR gene-editing system for the treatment of IDD comprises one or more sgRNAs targeting exon 2 of hIL6R. In some embodiments, the CRISPR gene-editing system for the treatment of IDD comprises one or more sgRNAs targeting exon 3 of hIL6R. In some embodiments, the CRISPR gene-editing system for the treatment of IDD comprises one or more sgRNAs targeting exon 4 of hIL6R. In some embodiments, the CRISPR gene-editing system for the treatment of IDD comprises one or more sgRNAs targeting exon 5 of hIL6R. In some embodiments, the CRISPR gene-editing system for the treatment of IDD comprises one or more sgRNAs targeting exon 6 of hIL6R. In some embodiments, the CRISPR gene-editing system for the treatment of IDD comprises one or more sgRNAs targeting exon 7 of hIL6R. In some embodiments, the CRISPR gene-editing system for the treatment of IDD comprises one or more sgRNAs targeting exon 8 of hIL6R. In some embodiments, the CRISPR gene-editing system for the treatment of IDD comprises one or more sgRNAs targeting exon 9 of hIL6R. In some embodiments, the CRISPR gene-editing system for the treatment of IDD comprises one or more sgRNAs targeting exon 10 of hIL6R. In some embodiments, the CRISPR gene-editing system for the treatment of IDD comprises one or more sgRNAs targeting exon 11 of hIL6R In some embodiments, the CRISPR gene-editing system for the treatment of IDD comprises one or more sgRNAs targeting exon 12 of hIL6R. In some embodiments, the CRISPR gene-editing system for the treatment of IDD comprises one or more sgRNAs targeting exon 13 of hIL6R. In some embodiments, the CRISPR gene-editing system for the treatment of IDD comprises one or more sgRNAs targeting exon 14 of hIL6R. In some embodiments, the CRISPR gene-editing system for the treatment of IDD comprises one or more sgRNAs targeting exon 15 of hIL6R.

In some embodiments, the CRISPR gene-editing system for the treatment of IDD comprises one or more sgRNAs targeting exon 1 of cIL6R. In some embodiments, the CRISPR gene-editing system for the treatment of IDD comprises one or more sgRNAs targeting exon 2 of cIL6R. In some embodiments, the CRISPR gene-editing system for the treatment of IDD comprises one or more sgRNAs targeting exon 3 of cIL6R. In some embodiments, the CRISPR gene-editing system for the treatment of IDD comprises one or more sgRNAs targeting exon 4 of cIL6R. In some embodiments, the CRISPR gene-editing system for the treatment of IDD comprises one or more sgRNAs targeting exon 5 of cIL6R. In some embodiments, the CRISPR gene-editing system for the treatment of IDD comprises one or more sgRNAs targeting exon 6 of cIL6R. In some embodiments, the CRISPR gene-editing system for the treatment of IDD comprises one or more sgRNAs targeting exon 7 of cIL6R. In some embodiments, the CRISPR gene-editing system for the treatment of IDD comprises one or more sgRNAs targeting exon 8 of cIL6R. In some embodiments, the CRISPR gene-editing system for the treatment of IDD comprises one or more sgRNAs targeting exon 9 of cIL6R. In some embodiments, the CRISPR gene-editing system for the treatment of IDD comprises one or more sgRNAs targeting exon 10 of cIL6R. In some embodiments, the CRISPR gene-editing system for the treatment of IDD comprises one or more sgRNAs targeting exon 11 of cIL6R. In some embodiments, the CRISPR gene-editing system for the treatment of IDD comprises one or more sgRNAs targeting exon 12 of cIL6R. In some embodiments, the CRISPR gene-editing system for the treatment of IDD comprises one or more sgRNAs targeting exon 13 of cIL6R.

In some embodiments, the CRISPR gene-editing system for the treatment of IDD comprises one or more sgRNAs targeting exon 1 of eIL6R. In some embodiments, the CRISPR gene-editing system for the treatment of IDD comprises one or more sgRNAs targeting exon 2 of eIL6R. In some embodiments, the CRISPR gene-editing system for the treatment of IDD comprises one or more sgRNAs targeting exon 3 of eIL6R. In some embodiments, the CRISPR gene-editing system for the treatment of IDD comprises one or more sgRNAs targeting exon 4 of eIL6R. In some embodiments, the CRISPR gene-editing system for the treatment of IDD comprises one or more sgRNAs targeting exon 5 of eIL6R. In some embodiments, the CRISPR gene-editing system for the treatment of IDD comprises one or more sgRNAs targeting exon 6 of eIL6R. In some embodiments, the CRISPR gene-editing system for the treatment of IDD comprises one or more sgRNAs targeting exon 7 of eIL6R. In some embodiments, the CRISPR gene-editing system for the treatment of IDD comprises one or more sgRNAs targeting exon 8 of eIL6R. In some embodiments, the CRISPR gene-editing system for the treatment of IDD comprises one or more sgRNAs targeting exon 9 of eIL6R. In some embodiments, the CRISPR gene-editing system for the treatment of IDD comprises one or more sgRNAs targeting exon 10 of eIL6R. In some embodiments, the CRISPR gene-editing system for the treatment of IDD comprises one or more sgRNAs targeting exon 11 of eIL6R.

In some embodiments, the CRISPR gene-editing system for the treatment of IDD comprises one or more sgRNAs targeting exon 1 of fIL6R. In some embodiments, the CRISPR gene-editing system for the treatment of IDD comprises one or more sgRNAs targeting exon 2 of fIL6R. In some embodiments, the CRISPR gene-editing system for the treatment of IDD comprises one or more sgRNAs targeting exon 3 of fIL6R. In some embodiments, the CRISPR gene-editing system for the treatment of IDD comprises one or more sgRNAs targeting exon 4 of fIL6R. In some embodiments, the CRISPR gene-editing system for the treatment of IDD comprises one or more sgRNAs targeting exon 5 of fIL6R. In some embodiments, the CRISPR gene-editing system for the treatment of IDD comprises one or more sgRNAs targeting exon 6 of fIL6R. In some embodiments, the CRISPR gene-editing system for the treatment of IDD comprises one or more sgRNAs targeting exon 7 of fIL6R. In some embodiments, the CRISPR gene-editing system for the treatment of IDD comprises one or more sgRNAs targeting exon 8 of fIL6R. In some embodiments, the CRISPR gene-editing system for the treatment of IDD comprises one or more sgRNAs targeting exon 9 of fIL6R. In some embodiments, the CRISPR gene-editing system for the treatment of IDD comprises one or more sgRNAs targeting exon 10 of fIL6R.

In some embodiments, the CRISPR gene-editing system for the treatment of IDD comprises one or more sgRNAs targeting IL6ST. In some embodiments, the CRISPR gene-editing system for the treatment of IDD comprises one or more sgRNAs targeting hIL6ST. In some embodiments, the CRISPR gene-editing system for the treatment of IDD comprises one or more sgRNAs targeting cIL6ST. In some embodiments, the CRISPR gene-editing system for the treatment of IDD comprises one or more sgRNAs targeting eIL6ST. In some embodiments, the CRISPR gene-editing system for the treatment of IDD comprises one or more sgRNAs targeting fIL6ST.

In some embodiments, the CRISPR gene-editing system for the treatment of IDD comprises one or more sgRNAs targeting exon 1 of hIL6ST. In some embodiments, the CRISPR gene-editing system for the treatment of IDD comprises one or more sgRNAs targeting exon 2 of hIL6ST. In some embodiments, the CRISPR gene-editing system for the treatment of IDD comprises one or more sgRNAs targeting exon 3 of hIL6ST. In some embodiments, the CRISPR gene-editing system for the treatment of IDD comprises one or more sgRNAs targeting exon 4 of hIL6ST. In some embodiments, the CRISPR gene-editing system for the treatment of IDD comprises one or more sgRNAs targeting exon 5 of hIL6ST. In some embodiments, the CRISPR gene-editing system for the treatment of IDD comprises one or more sgRNAs targeting exon 6 of hIL6ST. In some embodiments, the CRISPR gene-editing system for the treatment of IDD comprises one or more sgRNAs targeting exon 7 of hIL6ST. In some embodiments, the CRISPR gene-editing system for the treatment of IDD comprises one or more sgRNAs targeting exon 8 of hIL6ST. In some embodiments, the CRISPR gene-editing system for the treatment of IDD comprises one or more sgRNAs targeting exon 9 of hIL6ST. In some embodiments, the CRISPR gene-editing system for the treatment of IDD comprises one or more sgRNAs targeting exon 10 of hIL6ST. In some embodiments, the CRISPR gene-editing system for the treatment of IDD comprises one or more sgRNAs targeting exon 11 of hIL6ST. In some embodiments, the CRISPR gene-editing system for the treatment of IDD comprises one or more sgRNAs targeting exon 12 of hIL6ST. In some embodiments, the CRISPR gene-editing system for the treatment of IDD comprises one or more sgRNAs targeting exon 13 of hIL6ST. In some embodiments, the CRISPR gene-editing system for the treatment of IDD comprises one or more sgRNAs targeting exon 14 of hIL6ST. In some embodiments, the CRISPR gene-editing system for the treatment of IDD comprises one or more sgRNAs targeting exon 15 of hIL6ST. In some embodiments, the CRISPR gene-editing system for the treatment of IDD comprises one or more sgRNAs targeting exon 16 of hIL6ST. In some embodiments, the CRISPR gene-editing system for the treatment of IDD comprises one or more sgRNAs targeting exon 17 of hIL6ST. In some embodiments, the CRISPR gene-editing system for the treatment of IDD comprises one or more sgRNAs targeting exon 18 of hIL6ST. In some embodiments, the CRISPR gene-editing system for the treatment of IDD comprises one or more sgRNAs targeting exon 19 of hIL6ST.

In some embodiments, the CRISPR gene-editing system for the treatment of IDD comprises one or more sgRNAs targeting exon 1 of cIL6ST. In some embodiments, the CRISPR gene-editing system for the treatment of IDD comprises one or more sgRNAs targeting exon 2 of cIL6ST. In some embodiments, the CRISPR gene-editing system for the treatment of IDD comprises one or more sgRNAs targeting exon 3 of cIL6ST. In some embodiments, the CRISPR gene-editing system for the treatment of IDD comprises one or more sgRNAs targeting exon 4 of cIL6ST. In some embodiments, the CRISPR gene-editing system for the treatment of IDD comprises one or more sgRNAs targeting exon 5 of cIL6ST. In some embodiments, the CRISPR gene-editing system for the treatment of IDD comprises one or more sgRNAs targeting exon 6 of cIL6ST. In some embodiments, the CRISPR gene-editing system for the treatment of IDD comprises one or more sgRNAs targeting exon 7 of cIL6ST. In some embodiments, the CRISPR gene-editing system for the treatment of IDD comprises one or more sgRNAs targeting exon 8 of cIL6ST. In some embodiments, the CRISPR gene-editing system for the treatment of IDD comprises one or more sgRNAs targeting exon 9 of cIL6ST. In some embodiments, the CRISPR gene-editing system for the treatment of IDD comprises one or more sgRNAs targeting exon 10 of cIL6ST. In some embodiments, the CRISPR gene-editing system for the treatment of IDD comprises one or more sgRNAs targeting exon 11 of cIL6ST In some embodiments, the CRISPR gene-editing system for the treatment of IDD comprises one or more sgRNAs targeting exon 12 of cIL6ST. In some embodiments, the CRISPR gene-editing system for the treatment of IDD comprises one or more sgRNAs targeting exon 13 of cIL6ST. In some embodiments, the CRISPR gene-editing system for the treatment of IDD comprises one or more sgRNAs targeting exon 14 of cIL6ST. In some embodiments, the CRISPR gene-editing system for the treatment of IDD comprises one or more sgRNAs targeting exon 15 of cIL6ST. In some embodiments, the CRISPR gene-editing system for the treatment of IDD comprises one or more sgRNAs targeting exon 16 of cIL6ST. In some embodiments, the CRISPR gene-editing system for the treatment of IDD comprises one or more sgRNAs targeting exon 17 of cIL6ST. In some embodiments, the CRISPR gene-editing system for the treatment of IDD comprises one or more sgRNAs targeting exon 18 of cIL6ST.

In some embodiments, the CRISPR gene-editing system for the treatment of IDD comprises one or more sgRNAs targeting exon 1 of eIL6ST. In some embodiments, the CRISPR gene-editing system for the treatment of IDD comprises one or more sgRNAs targeting exon 2 of eIL6ST. In some embodiments, the CRISPR gene-editing system for the treatment of IDD comprises one or more sgRNAs targeting exon 3 of eIL6ST. In some embodiments, the CRISPR gene-editing system for the treatment of IDD comprises one or more sgRNAs targeting exon 4 of eIL6ST. In some embodiments, the CRISPR gene-editing system for the treatment of IDD comprises one or more sgRNAs targeting exon 5 of eIL6ST. In some embodiments, the CRISPR gene-editing system for the treatment of IDD comprises one or more sgRNAs targeting exon 6 of eIL6ST. In some embodiments, the CRISPR gene-editing system for the treatment of IDD comprises one or more sgRNAs targeting exon 7 of eIL6ST. In some embodiments, the CRISPR gene-editing system for the treatment of IDD comprises one or more sgRNAs targeting exon 8 of eIL6ST. In some embodiments, the CRISPR gene-editing system for the treatment of IDD comprises one or more sgRNAs targeting exon 9 of eIL6ST. In some embodiments, the CRISPR gene-editing system for the treatment of IDD comprises one or more sgRNAs targeting exon 10 of eIL6ST. In some embodiments, the CRISPR gene-editing system for the treatment of IDD comprises one or more sgRNAs targeting exon 11 of eIL6ST In some embodiments, the CRISPR gene-editing system for the treatment of IDD comprises one or more sgRNAs targeting exon 12 of eIL6ST. In some embodiments, the CRISPR gene-editing system for the treatment of IDD comprises one or more sgRNAs targeting exon 13 of eIL6ST. In some embodiments, the CRISPR gene-editing system for the treatment of IDD comprises one or more sgRNAs targeting exon 14 of eIL6ST. In some embodiments, the CRISPR gene-editing system for the treatment of IDD comprises one or more sgRNAs targeting exon 15 of eIL6ST. In some embodiments, the CRISPR gene-editing system for the treatment of IDD comprises one or more sgRNAs targeting exon 16 of eIL6ST. In some embodiments, the CRISPR gene-editing system for the treatment of IDD comprises one or more sgRNAs targeting exon 17 of eIL6ST. In some embodiments, the CRISPR gene-editing system for the treatment of IDD comprises one or more sgRNAs targeting exon 18 of eIL6ST.

In some embodiments, the CRISPR gene-editing system for the treatment of IDD comprises one or more sgRNAs targeting exon 1 of fIL6ST. In some embodiments, the CRISPR gene-editing system for the treatment of IDD comprises one or more sgRNAs targeting exon 2 of fIL6ST. In some embodiments, the CRISPR gene-editing system for the treatment of IDD comprises one or more sgRNAs targeting exon 3 of fIL6ST. In some embodiments, the CRISPR gene-editing system for the treatment of IDD comprises one or more sgRNAs targeting exon 4 of fIL6ST. In some embodiments, the CRISPR gene-editing system for the treatment of IDD comprises one or more sgRNAs targeting exon 5 of fIL6ST. In some embodiments, the CRISPR gene-editing system for the treatment of IDD comprises one or more sgRNAs targeting exon 6 of fIL6ST. In some embodiments, the CRISPR gene-editing system for the treatment of IDD comprises one or more sgRNAs targeting exon 7 of fIL6ST. In some embodiments, the CRISPR gene-editing system for the treatment of IDD comprises one or more sgRNAs targeting exon 8 of fIL6ST. In some embodiments, the CRISPR gene-editing system for the treatment of IDD comprises one or more sgRNAs targeting exon 9 of fIL6ST. In some embodiments, the CRISPR gene-editing system for the treatment of IDD comprises one or more sgRNAs targeting exon 10 of fIL6ST. In some embodiments, the CRISPR gene-editing system for the treatment of IDD comprises one or more sgRNAs targeting exon 11 of fIL6ST In some embodiments, the CRISPR gene-editing system for the treatment of IDD comprises one or more sgRNAs targeting exon 12 of fIL6ST. In some embodiments, the CRISPR gene-editing system for the treatment of IDD comprises one or more sgRNAs targeting exon 13 of fIL6ST. In some embodiments, the CRISPR gene-editing system for the treatment of IDD comprises one or more sgRNAs targeting exon 14 of fIL6ST. In some embodiments, the CRISPR gene-editing system for the treatment of IDD comprises one or more sgRNAs targeting exon 15 of fIL6ST. In some embodiments, the CRISPR gene-editing system for the treatment of IDD comprises one or more sgRNAs targeting exon 16 of fIL6ST. In some embodiments, the CRISPR gene-editing system for the treatment of IDD comprises one or more sgRNAs targeting exon 17 of fIL6ST. In some embodiments, the CRISPR gene-editing system for the treatment of IDD comprises one or more sgRNAs targeting exon 18 of fIL6ST. In some embodiments, the CRISPR gene-editing system for the treatment of IDD comprises one or more sgRNAs targeting exon 19 of fIL6ST. In some embodiments, the CRISPR gene-editing system for the treatment of IDD comprises one or more sgRNAs targeting exon 20 of fIL6ST.

In some embodiments, the CRISPR gene-editing system for the treatment of IDD comprises one or more sgRNAs targeting TNFRSF1A. In some embodiments, the CRISPR gene-editing system for the treatment of IDD comprises one or more sgRNAs targeting hTNFRSF1A. In some embodiments, the CRISPR gene-editing system for the treatment of IDD comprises one or more sgRNAs targeting cTNFRSF1A. In some embodiments, the CRISPR gene-editing system for the treatment of IDD comprises one or more sgRNAs targeting eTNFRSF1A. In some embodiments, the CRISPR gene-editing system for the treatment of IDD comprises one or more sgRNAs targeting fTNFRSF1A.

In some embodiments, the CRISPR gene-editing system for the treatment of IDD comprises one or more sgRNAs targeting exon 1 of hTNFRSF1A. In some embodiments, the CRISPR gene-editing system for the treatment of IDD comprises one or more sgRNAs targeting exon 2 of hTNFRSF1A. In some embodiments, the CRISPR gene-editing system for the treatment of IDD comprises one or more sgRNAs targeting exon 3 of hTNFRSF1A. In some embodiments, the CRISPR gene-editing system for the treatment of IDD comprises one or more sgRNAs targeting exon 4 of hTNFRSF1A. In some embodiments, the CRISPR gene-editing system for the treatment of IDD comprises one or more sgRNAs targeting exon 5 of hTNFRSF1A. In some embodiments, the CRISPR gene-editing system for the treatment of IDD comprises one or more sgRNAs targeting exon 6 of hTNFRSF1A. In some embodiments, the CRISPR gene-editing system for the treatment of IDD comprises one or more sgRNAs targeting exon 7 of hTNFRSF1A. In some embodiments, the CRISPR gene-editing system for the treatment of IDD comprises one or more sgRNAs targeting exon 8 of hTNFRSF1A. In some embodiments, the CRISPR gene-editing system for the treatment of IDD comprises one or more sgRNAs targeting exon 9 of hTNFRSF1A. In some embodiments, the CRISPR gene-editing system for the treatment of IDD comprises one or more sgRNAs targeting exon 10 of hTNFRSF1A. In some embodiments, the CRISPR gene-editing system for the treatment of IDD comprises one or more sgRNAs targeting exon 11 of hTNFRSF1A.

In some embodiments, the CRISPR gene-editing system for the treatment of IDD comprises one or more sgRNAs targeting exon 1 of cTNFRSF1A. In some embodiments, the CRISPR gene-editing system for the treatment of IDD comprises one or more sgRNAs targeting exon 2 of cTNFRSF1A. In some embodiments, the CRISPR gene-editing system for the treatment of IDD comprises one or more sgRNAs targeting exon 3 of cTNFRSF1A. In some embodiments, the CRISPR gene-editing system for the treatment of IDD comprises one or more sgRNAs targeting exon 4 of cTNFRSF1A. In some embodiments, the CRISPR gene-editing system for the treatment of IDD comprises one or more sgRNAs targeting exon 5 of cTNFRSF1A. In some embodiments, the CRISPR gene-editing system for the treatment of IDD comprises one or more sgRNAs targeting exon 6 of cTNFRSF1A. In some embodiments, the CRISPR gene-editing system for the treatment of IDD comprises one or more sgRNAs targeting exon 7 of cTNFRSF1A. In some embodiments, the CRISPR gene-editing system for the treatment of IDD comprises one or more sgRNAs targeting exon 8 of cTNFRSF1A. In some embodiments, the CRISPR gene-editing system for the treatment of IDD comprises one or more sgRNAs targeting exon 9 of cTNFRSF1A. In some embodiments, the CRISPR gene-editing system for the treatment of IDD comprises one or more sgRNAs targeting exon 10 of cTNFRSF1A. In some embodiments, the CRISPR gene-editing system for the treatment of IDD comprises one or more sgRNAs targeting exon 11 of cTNFRSF1A.

In some embodiments, the CRISPR gene-editing system for the treatment of IDD comprises one or more sgRNAs targeting exon 1 of eTNFRSF1A. In some embodiments, the CRISPR gene-editing system for the treatment of IDD comprises one or more sgRNAs targeting exon 2 of eTNFRSF1A. In some embodiments, the CRISPR gene-editing system for the treatment of IDD comprises one or more sgRNAs targeting exon 3 of eTNFRSF1A. In some embodiments, the CRISPR gene-editing system for the treatment of IDD comprises one or more sgRNAs targeting exon 4 of eTNFRSF1A. In some embodiments, the CRISPR gene-editing system for the treatment of IDD comprises one or more sgRNAs targeting exon 5 of eTNFRSF1A. In some embodiments, the CRISPR gene-editing system for the treatment of IDD comprises one or more sgRNAs targeting exon 6 of eTNFRSF1A. In some embodiments, the CRISPR gene-editing system for the treatment of IDD comprises one or more sgRNAs targeting exon 7 of eTNFRSF1A. In some embodiments, the CRISPR gene-editing system for the treatment of IDD comprises one or more sgRNAs targeting exon 8 of eTNFRSF1A. In some embodiments, the CRISPR gene-editing system for the treatment of IDD comprises one or more sgRNAs targeting exon 9 of eTNFRSF1A. In some embodiments, the CRISPR gene-editing system for the treatment of IDD comprises one or more sgRNAs targeting exon 10 of eTNFRSF1A.

In some embodiments, the CRISPR gene-editing system for the treatment of IDD comprises one or more sgRNAs targeting exon 1 of fTNFRSF1A. In some embodiments, the CRISPR gene-editing system for the treatment of IDD comprises one or more sgRNAs targeting exon 2 of fTNFRSF1A. In some embodiments, the CRISPR gene-editing system for the treatment of IDD comprises one or more sgRNAs targeting exon 3 of fTNFRSF1A. In some embodiments, the CRISPR gene-editing system for the treatment of IDD comprises one or more sgRNAs targeting exon 4 of fINFRSF1A. In some embodiments, the CRISPR gene-editing system for the treatment of IDD comprises one or more sgRNAs targeting exon 5 of fTNFRSF1A. In some embodiments, the CRISPR gene-editing system for the treatment of IDD comprises one or more sgRNAs targeting exon 6 of fTNFRSF1A. In some embodiments, the CRISPR gene-editing system for the treatment of IDD comprises one or more sgRNAs targeting exon 7 of fTNFRSF1A. In some embodiments, the CRISPR gene-editing system for the treatment of IDD comprises one or more sgRNAs targeting exon 8 of fTNFRSF1A. In some embodiments, the CRISPR gene-editing system for the treatment of IDD comprises one or more sgRNAs targeting exon 9 of fINFRSF1A. In some embodiments, the CRISPR gene-editing system for the treatment of IDD comprises one or more sgRNAs targeting exon 10 of fTNFRSF1A.

In some embodiments, the CRISPR gene-editing system for the treatment of IDD comprises one or more sgRNAs targeting TNFRSF1B. In some embodiments, the CRISPR gene-editing system for the treatment of IDD comprises one or more sgRNAs targeting hTNFRSF1B. In some embodiments, the CRISPR gene-editing system for the treatment of IDD comprises one or more sgRNAs targeting cTNFRSF1B. In some embodiments, the CRISPR gene-editing system for the treatment of IDD comprises one or more sgRNAs targeting eTNFRSF1B. In some embodiments, the CRISPR gene-editing system for the treatment of IDD comprises one or more sgRNAs targeting fTNFRSF1B.

In some embodiments, the CRISPR gene-editing system for the treatment of IDD comprises one or more sgRNAs targeting exon 1 of hTNFRSF1B. In some embodiments, the CRISPR gene-editing system for the treatment of IDD comprises one or more sgRNAs targeting exon 2 of hTNFRSF1B. In some embodiments, the CRISPR gene-editing system for the treatment of IDD comprises one or more sgRNAs targeting exon 3 of hTNFRSF1B. In some embodiments, the CRISPR gene-editing system for the treatment of IDD comprises one or more sgRNAs targeting exon 4 of hTNFRSF1B. In some embodiments, the CRISPR gene-editing system for the treatment of IDD comprises one or more sgRNAs targeting exon 5 of hTNFRSF1B. In some embodiments, the CRISPR gene-editing system for the treatment of IDD comprises one or more sgRNAs targeting exon 6 of hTNFRSF1B. In some embodiments, the CRISPR gene-editing system for the treatment of IDD comprises one or more sgRNAs targeting exon 7 of hTNFRSF1B. In some embodiments, the CRISPR gene-editing system for the treatment of IDD comprises one or more sgRNAs targeting exon 8 of hTNFRSF1B. In some embodiments, the CRISPR gene-editing system for the treatment of IDD comprises one or more sgRNAs targeting exon 9 of hTNFRSF1B. In some embodiments, the CRISPR gene-editing system for the treatment of IDD comprises one or more sgRNAs targeting exon 10 of hTNFRSF1B. In some embodiments, the CRISPR gene-editing system for the treatment of IDD comprises one or more sgRNAs targeting exon 11 of hTNFRSF1B In some embodiments, the CRISPR gene-editing system for the treatment of IDD comprises one or more sgRNAs targeting exon 12 of hTNFRSF1B. In some embodiments, the CRISPR gene-editing system for the treatment of IDD comprises one or more sgRNAs targeting exon 13 of hTNFRSF1B.

In some embodiments, the CRISPR gene-editing system for the treatment of IDD comprises one or more sgRNAs targeting exon 1 of cTNFRSF1B. In some embodiments, the CRISPR gene-editing system for the treatment of IDD comprises one or more sgRNAs targeting exon 2 of cTNFRSF1B. In some embodiments, the CRISPR gene-editing system for the treatment of IDD comprises one or more sgRNAs targeting exon 3 of cTNFRSF1B. In some embodiments, the CRISPR gene-editing system for the treatment of IDD comprises one or more sgRNAs targeting exon 4 of cTNFRSF1B. In some embodiments, the CRISPR gene-editing system for the treatment of IDD comprises one or more sgRNAs targeting exon 5 of cTNFRSF1B. In some embodiments, the CRISPR gene-editing system for the treatment of IDD comprises one or more sgRNAs targeting exon 6 of cTNFRSF1B. In some embodiments, the CRISPR gene-editing system for the treatment of IDD comprises one or more sgRNAs targeting exon 7 of cTNFRSF1B. In some embodiments, the CRISPR gene-editing system for the treatment of IDD comprises one or more sgRNAs targeting exon 8 of cTNFRSF1B. In some embodiments, the CRISPR gene-editing system for the treatment of IDD comprises one or more sgRNAs targeting exon 9 of cTNFRSF1B. In some embodiments, the CRISPR gene-editing system for the treatment of IDD comprises one or more sgRNAs targeting exon 10 of cTNFRSF1B. In some embodiments, the CRISPR gene-editing system for the treatment of IDD comprises one or more sgRNAs targeting exon 11 of cTNFRSF1B.

In some embodiments, the CRISPR gene-editing system for the treatment of IDD comprises one or more sgRNAs targeting exon 1 of eTNFRSF1B. In some embodiments, the CRISPR gene-editing system for the treatment of IDD comprises one or more sgRNAs targeting exon 2 of eTNFRSF1B. In some embodiments, the CRISPR gene-editing system for the treatment of IDD comprises one or more sgRNAs targeting exon 3 of eTNFRSF1B. In some embodiments, the CRISPR gene-editing system for the treatment of IDD comprises one or more sgRNAs targeting exon 4 of eTNFRSF1B. In some embodiments, the CRISPR gene-editing system for the treatment of IDD comprises one or more sgRNAs targeting exon 5 of eTNFRSF1B. In some embodiments, the CRISPR gene-editing system for the treatment of IDD comprises one or more sgRNAs targeting exon 6 of eTNFRSF1B. In some embodiments, the CRISPR gene-editing system for the treatment of IDD comprises one or more sgRNAs targeting exon 7 of eTNFRSF1B. In some embodiments, the CRISPR gene-editing system for the treatment of IDD comprises one or more sgRNAs targeting exon 8 of eTNFRSF1B. In some embodiments, the CRISPR gene-editing system for the treatment of IDD comprises one or more sgRNAs targeting exon 9 of eTNFRSF1B. In some embodiments, the CRISPR gene-editing system for the treatment of IDD comprises one or more sgRNAs targeting exon 10 of eTNFRSF1B. In some embodiments, the CRISPR gene-editing system for the treatment of IDD comprises one or more sgRNAs targeting exon 11 of eTNFRSF1B.

In some embodiments, the CRISPR gene-editing system for the treatment of IDD comprises one or more sgRNAs targeting exon 1 of fTNFRSF1B. In some embodiments, the CRISPR gene-editing system for the treatment of IDD comprises one or more sgRNAs targeting exon 2 of fTNFRSF1B. In some embodiments, the CRISPR gene-editing system for the treatment of IDD comprises one or more sgRNAs targeting exon 3 of fTNFRSF1B. In some embodiments, the CRISPR gene-editing system for the treatment of IDD comprises one or more sgRNAs targeting exon 4 of fTNFRSF1B. In some embodiments, the CRISPR gene-editing system for the treatment of IDD comprises one or more sgRNAs targeting exon 5 of fTNFRSF1B. In some embodiments, the CRISPR gene-editing system for the treatment of IDD comprises one or more sgRNAs targeting exon 6 of fTNFRSF1B. In some embodiments, the CRISPR gene-editing system for the treatment of IDD comprises one or more sgRNAs targeting exon 7 of fTNFRSF1B. In some embodiments, the CRISPR gene-editing system for the treatment of IDD comprises one or more sgRNAs targeting exon 8 of fTNFRSF1B. In some embodiments, the CRISPR gene-editing system for the treatment of IDD comprises one or more sgRNAs targeting exon 9 of fTNFRSF1B. In some embodiments, the CRISPR gene-editing system for the treatment of IDD comprises one or more sgRNAs targeting exon 10 of fTNFRSF1B.

In some embodiments, the CRISPR gene-editing system for the treatment of IDD comprises one or more sgRNAs targeting TNFRSF3. In some embodiments, the CRISPR gene-editing system for the treatment of IDD comprises one or more sgRNAs targeting hTNFRSF3. In some embodiments, the CRISPR gene-editing system for the treatment of IDD comprises one or more sgRNAs targeting cTNFRSF3. In some embodiments, the CRISPR gene-editing system for the treatment of IDD comprises one or more sgRNAs targeting eTNFRSF3. In some embodiments, the CRISPR gene-editing system for the treatment of IDD comprises one or more sgRNAs targeting fTNFRSF3.

In some embodiments, the CRISPR gene-editing system for the treatment of IDD comprises one or more sgRNAs targeting exon 1 of hTNFRSF3. In some embodiments, the CRISPR gene-editing system for the treatment of IDD comprises one or more sgRNAs targeting exon 2 of hTNFRSF3. In some embodiments, the CRISPR gene-editing system for the treatment of IDD comprises one or more sgRNAs targeting exon 3 of hTNFRSF3. In some embodiments, the CRISPR gene-editing system for the treatment of IDD comprises one or more sgRNAs targeting exon 4 of hTNFRSF3. In some embodiments, the CRISPR gene-editing system for the treatment of IDD comprises one or more sgRNAs targeting exon 5 of hTNFRSF3. In some embodiments, the CRISPR gene-editing system for the treatment of IDD comprises one or more sgRNAs targeting exon 6 of hTNFRSF3. In some embodiments, the CRISPR gene-editing system for the treatment of IDD comprises one or more sgRNAs targeting exon 7 of hTNFRSF3. In some embodiments, the CRISPR gene-editing system for the treatment of IDD comprises one or more sgRNAs targeting exon 8 of hTNFRSF3. In some embodiments, the CRISPR gene-editing system for the treatment of IDD comprises one or more sgRNAs targeting exon 9 of hTNFRSF3. In some embodiments, the CRISPR gene-editing system for the treatment of IDD comprises one or more sgRNAs targeting exon 10 of hTNFRSF3. In some embodiments, the CRISPR gene-editing system for the treatment of IDD comprises one or more sgRNAs targeting exon 11 of hTNFRSF3. In some embodiments, the CRISPR gene-editing system for the treatment of IDD comprises one or more sgRNAs targeting exon 12 of hTNFRSF3.

In some embodiments, the CRISPR gene-editing system for the treatment of IDD comprises one or more sgRNAs targeting exon 1 of cTNFRSF3. In some embodiments, the CRISPR gene-editing system for the treatment of IDD comprises one or more sgRNAs targeting exon 2 of cTNFRSF3. In some embodiments, the CRISPR gene-editing system for the treatment of IDD comprises one or more sgRNAs targeting exon 3 of cTNFRSF3. In some embodiments, the CRISPR gene-editing system for the treatment of IDD comprises one or more sgRNAs targeting exon 4 of cTNFRSF3. In some embodiments, the CRISPR gene-editing system for the treatment of IDD comprises one or more sgRNAs targeting exon 5 of cTNFRSF3. In some embodiments, the CRISPR gene-editing system for the treatment of IDD comprises one or more sgRNAs targeting exon 6 of cTNFRSF3. In some embodiments, the CRISPR gene-editing system for the treatment of IDD comprises one or more sgRNAs targeting exon 7 of cTNFRSF3. In some embodiments, the CRISPR gene-editing system for the treatment of IDD comprises one or more sgRNAs targeting exon 8 of cTNFRSF3. In some embodiments, the CRISPR gene-editing system for the treatment of IDD comprises one or more sgRNAs targeting exon 9 of cTNFRSF3. In some embodiments, the CRISPR gene-editing system for the treatment of IDD comprises one or more sgRNAs targeting exon 10 of cTNFRSF3.

In some embodiments, the CRISPR gene-editing system for the treatment of IDD comprises one or more sgRNAs targeting exon 1 of eTNFRSF3. In some embodiments, the CRISPR gene-editing system for the treatment of IDD comprises one or more sgRNAs targeting exon 2 of eTNFRSF3. In some embodiments, the CRISPR gene-editing system for the treatment of IDD comprises one or more sgRNAs targeting exon 3 of eTNFRSF3. In some embodiments, the CRISPR gene-editing system for the treatment of IDD comprises one or more sgRNAs targeting exon 4 of eTNFRSF3. In some embodiments, the CRISPR gene-editing system for the treatment of IDD comprises one or more sgRNAs targeting exon 5 of eTNFRSF3. In some embodiments, the CRISPR gene-editing system for the treatment of IDD comprises one or more sgRNAs targeting exon 6 of eTNFRSF3. In some embodiments, the CRISPR gene-editing system for the treatment of IDD comprises one or more sgRNAs targeting exon 7 of eTNFRSF3. In some embodiments, the CRISPR gene-editing system for the treatment of IDD comprises one or more sgRNAs targeting exon 8 of eTNFRSF3. In some embodiments, the CRISPR gene-editing system for the treatment of IDD comprises one or more sgRNAs targeting exon 9 of eTNFRSF3. In some embodiments, the CRISPR gene-editing system for the treatment of IDD comprises one or more sgRNAs targeting exon 10 of eTNFRSF3.

In some embodiments, the CRISPR gene-editing system for the treatment of IDD comprises one or more sgRNAs targeting exon 1 of fTNFRSF3. In some embodiments, the CRISPR gene-editing system for the treatment of IDD comprises one or more sgRNAs targeting exon 2 of fTNFRSF3. In some embodiments, the CRISPR gene-editing system for the treatment of IDD comprises one or more sgRNAs targeting exon 3 of fTNFRSF3. In some embodiments, the CRISPR gene-editing system for the treatment of IDD comprises one or more sgRNAs targeting exon 4 of fTNFRSF3. In some embodiments, the CRISPR gene-editing system for the treatment of IDD comprises one or more sgRNAs targeting exon 5 of fTNFRSF3. In some embodiments, the CRISPR gene-editing system for the treatment of IDD comprises one or more sgRNAs targeting exon 6 of fTNFRSF3. In some embodiments, the CRISPR gene-editing system for the treatment of IDD comprises one or more sgRNAs targeting exon 7 of fTNFRSF3. In some embodiments, the CRISPR gene-editing system for the treatment of IDD comprises one or more sgRNAs targeting exon 8 of fTNFRSF3. In some embodiments, the CRISPR gene-editing system for the treatment of IDD comprises one or more sgRNAs targeting exon 9 of fTNFRSF3. In some embodiments, the CRISPR gene-editing system for the treatment of IDD comprises one or more sgRNAs targeting exon 10 of fTNFRSF3.

In some embodiments, the CRISPR gene-editing system for the treatment of IDD comprises one or more sgRNAs targeting TNFRSF4. In some embodiments, the CRISPR gene-editing system for the treatment of IDD comprises one or more sgRNAs targeting hTNFRSF4. In some embodiments, the CRISPR gene-editing system for the treatment of IDD comprises one or more sgRNAs targeting cTNFRSF4. In some embodiments, the CRISPR gene-editing system for the treatment of IDD comprises one or more sgRNAs targeting eTNFRSF4. In some embodiments, the CRISPR gene-editing system for the treatment of IDD comprises one or more sgRNAs targeting fTNFRSF4.

In some embodiments, the CRISPR gene-editing system for the treatment of IDD comprises one or more sgRNAs targeting exon 1 of hTNFRSF4. In some embodiments, the CRISPR gene-editing system for the treatment of IDD comprises one or more sgRNAs targeting exon 2 of hTNFRSF4. In some embodiments, the CRISPR gene-editing system for the treatment of IDD comprises one or more sgRNAs targeting exon 3 of hTNFRSF4. In some embodiments, the CRISPR gene-editing system for the treatment of IDD comprises one or more sgRNAs targeting exon 4 of hTNFRSF4. In some embodiments, the CRISPR gene-editing system for the treatment of IDD comprises one or more sgRNAs targeting exon 5 of hTNFRSF4. In some embodiments, the CRISPR gene-editing system for the treatment of IDD comprises one or more sgRNAs targeting exon 6 of hTNFRSF4.

In some embodiments, the CRISPR gene-editing system for the treatment of IDD comprises one or more sgRNAs targeting exon 1 of cTNFRSF4. In some embodiments, the CRISPR gene-editing system for the treatment of IDD comprises one or more sgRNAs targeting exon 2 of cTNFRSF4. In some embodiments, the CRISPR gene-editing system for the treatment of IDD comprises one or more sgRNAs targeting exon 3 of cTNFRSF4. In some embodiments, the CRISPR gene-editing system for the treatment of IDD comprises one or more sgRNAs targeting exon 4 of cTNFRSF4. In some embodiments, the CRISPR gene-editing system for the treatment of IDD comprises one or more sgRNAs targeting exon 5 of cTNFRSF4. In some embodiments, the CRISPR gene-editing system for the treatment of IDD comprises one or more sgRNAs targeting exon 6 of cTNFRSF4. In some embodiments, the CRISPR gene-editing system for the treatment of IDD comprises one or more sgRNAs targeting exon 7 of cTNFRSF4.

In some embodiments, the CRISPR gene-editing system for the treatment of IDD comprises one or more sgRNAs targeting exon 1 of eTNFRSF4. In some embodiments, the CRISPR gene-editing system for the treatment of IDD comprises one or more sgRNAs targeting exon 2 of eTNFRSF4. In some embodiments, the CRISPR gene-editing system for the treatment of IDD comprises one or more sgRNAs targeting exon 3 of eTNFRSF4. In some embodiments, the CRISPR gene-editing system for the treatment of IDD comprises one or more sgRNAs targeting exon 4 of eTNFRSF4. In some embodiments, the CRISPR gene-editing system for the treatment of IDD comprises one or more sgRNAs targeting exon 5 of eTNFRSF4. In some embodiments, the CRISPR gene-editing system for the treatment of IDD comprises one or more sgRNAs targeting exon 6 of eTNFRSF4. In some embodiments, the CRISPR gene-editing system for the treatment of IDD comprises one or more sgRNAs targeting exon 7 of eTNFRSF4.

In some embodiments, the CRISPR gene-editing system for the treatment of IDD comprises one or more sgRNAs targeting exon 1 of fTNFRSF4. In some embodiments, the CRISPR gene-editing system for the treatment of IDD comprises one or more sgRNAs targeting exon 2 of fTNFRSF4. In some embodiments, the CRISPR gene-editing system for the treatment of IDD comprises one or more sgRNAs targeting exon 3 of fTNFRSF4. In some embodiments, the CRISPR gene-editing system for the treatment of IDD comprises one or more sgRNAs targeting exon 4 of fTNFRSF4. In some embodiments, the CRISPR gene-editing system for the treatment of IDD comprises one or more sgRNAs targeting exon 5 of fTNFRSF4. In some embodiments, the CRISPR gene-editing system for the treatment of IDD comprises one or more sgRNAs targeting exon 6 of fTNFRSF4. In some embodiments, the CRISPR gene-editing system for the treatment of IDD comprises one or more sgRNAs targeting exon 7 of fTNFRSF4.

In some embodiments, the CRISPR gene-editing system for the treatment of IDD comprises one or more sgRNAs targeting TNFRSF11A. In some embodiments, the CRISPR gene-editing system for the treatment of IDD comprises one or more sgRNAs targeting hTNFRSF11A. In some embodiments, the CRISPR gene-editing system for the treatment of IDD comprises one or more sgRNAs targeting cTNFRSF11A. In some embodiments, the CRISPR gene-editing system for the treatment of IDD comprises one or more sgRNAs targeting eTNFRSF11A. In some embodiments, the CRISPR gene-editing system for the treatment of IDD comprises one or more sgRNAs targeting fTNFRSF11A.

In some embodiments, the CRISPR gene-editing system for the treatment of IDD comprises one or more sgRNAs targeting exon 1 of hTNFRSF11A. In some embodiments, the CRISPR gene-editing system for the treatment of IDD comprises one or more sgRNAs targeting exon 2 of hTNFRSF11A. In some embodiments, the CRISPR gene-editing system for the treatment of IDD comprises one or more sgRNAs targeting exon 3 of hTNFRSF11A. In some embodiments, the CRISPR gene-editing system for the treatment of IDD comprises one or more sgRNAs targeting exon 4 of hTNFRSF11A. In some embodiments, the CRISPR gene-editing system for the treatment of IDD comprises one or more sgRNAs targeting exon 5 of hTNFRSF11A. In some embodiments, the CRISPR gene-editing system for the treatment of IDD comprises one or more sgRNAs targeting exon 6 of hTNFRSF11A. In some embodiments, the CRISPR gene-editing system for the treatment of IDD comprises one or more sgRNAs targeting exon 7 of hTNFRSF11A. In some embodiments, the CRISPR gene-editing system for the treatment of IDD comprises one or more sgRNAs targeting exon 8 of hTNFRSF11A. In some embodiments, the CRISPR gene-editing system for the treatment of IDD comprises one or more sgRNAs targeting exon 9 of hTNFRSF11A. In some embodiments, the CRISPR gene-editing system for the treatment of IDD comprises one or more sgRNAs targeting exon 10 of hTNFRSF11A. In some embodiments, the CRISPR gene-editing system for the treatment of IDD comprises one or more sgRNAs targeting exon 11 of hTNFRSF11A. In some embodiments, the CRISPR gene-editing system for the treatment of IDD comprises one or more sgRNAs targeting exon 12 of hTNFRSF11A.

In some embodiments, the CRISPR gene-editing system for the treatment of IDD comprises one or more sgRNAs targeting exon 1 of cTNFRSF11A. In some embodiments, the CRISPR gene-editing system for the treatment of IDD comprises one or more sgRNAs targeting exon 2 of cTNFRSF11A. In some embodiments, the CRISPR gene-editing system for the treatment of IDD comprises one or more sgRNAs targeting exon 3 of cTNFRSF11A. In some embodiments, the CRISPR gene-editing system for the treatment of IDD comprises one or more sgRNAs targeting exon 4 of cTNFRSF11A. In some embodiments, the CRISPR gene-editing system for the treatment of IDD comprises one or more sgRNAs targeting exon 5 of cTNFRSF11A. In some embodiments, the CRISPR gene-editing system for the treatment of IDD comprises one or more sgRNAs targeting exon 6 of cTNFRSF11A. In some embodiments, the CRISPR gene-editing system for the treatment of IDD comprises one or more sgRNAs targeting exon 7 of cTNFRSF11A. In some embodiments, the CRISPR gene-editing system for the treatment of IDD comprises one or more sgRNAs targeting exon 8 of cTNFRSF11A. In some embodiments, the CRISPR gene-editing system for the treatment of IDD comprises one or more sgRNAs targeting exon 9 of cTNFRSF11A. In some embodiments, the CRISPR gene-editing system for the treatment of IDD comprises one or more sgRNAs targeting exon 10 of cTNFRSF11A. In some embodiments, the CRISPR gene-editing system for the treatment of IDD comprises one or more sgRNAs targeting exon 11 of cTNFRSF11A.

In some embodiments, the CRISPR gene-editing system for the treatment of IDD comprises one or more sgRNAs targeting exon 1 of eTNFRSF11A. In some embodiments, the CRISPR gene-editing system for the treatment of IDD comprises one or more sgRNAs targeting exon 2 of eTNFRSF11A. In some embodiments, the CRISPR gene-editing system for the treatment of IDD comprises one or more sgRNAs targeting exon 3 of eTNFRSF11A. In some embodiments, the CRISPR gene-editing system for the treatment of IDD comprises one or more sgRNAs targeting exon 4 of eTNFRSF11A. In some embodiments, the CRISPR gene-editing system for the treatment of IDD comprises one or more sgRNAs targeting exon 5 of eTNFRSF11A. In some embodiments, the CRISPR gene-editing system for the treatment of IDD comprises one or more sgRNAs targeting exon 6 of eTNFRSF11A. In some embodiments, the CRISPR gene-editing system for the treatment of IDD comprises one or more sgRNAs targeting exon 7 of eTNFRSF11A. In some embodiments, the CRISPR gene-editing system for the treatment of IDD comprises one or more sgRNAs targeting exon 8 of eTNFRSF11A. In some embodiments, the CRISPR gene-editing system for the treatment of IDD comprises one or more sgRNAs targeting exon 9 of eTNFRSF11A. In some embodiments, the CRISPR gene-editing system for the treatment of IDD comprises one or more sgRNAs targeting exon 10 of eTNFRSF11A.

In some embodiments, the CRISPR gene-editing system for the treatment of IDD comprises one or more sgRNAs targeting exon 1 of fTNFRSF11A. In some embodiments, the CRISPR gene-editing system for the treatment of IDD comprises one or more sgRNAs targeting exon 2 of fTNFRSF11A. In some embodiments, the CRISPR gene-editing system for the treatment of IDD comprises one or more sgRNAs targeting exon 3 of fTNFRSF11A. In some embodiments, the CRISPR gene-editing system for the treatment of IDD comprises one or more sgRNAs targeting exon 4 of fTNFRSF11A. In some embodiments, the CRISPR gene-editing system for the treatment of IDD comprises one or more sgRNAs targeting exon 5 of fTNFRSF11A. In some embodiments, the CRISPR gene-editing system for the treatment of IDD comprises one or more sgRNAs targeting exon 6 of fTNFRSF11A. In some embodiments, the CRISPR gene-editing system for the treatment of IDD comprises one or more sgRNAs targeting exon 7 of fTNFRSF11A. In some embodiments, the CRISPR gene-editing system for the treatment of IDD comprises one or more sgRNAs targeting exon 8 of fTNFRSF11A. In some embodiments, the CRISPR gene-editing system for the treatment of IDD comprises one or more sgRNAs targeting exon 9 of fTNFRSF11A. In some embodiments, the CRISPR gene-editing system for the treatment of IDD comprises one or more sgRNAs targeting exon 10 of fTNFRSF11A. In some embodiments, the CRISPR gene-editing system for the treatment of IDD comprises one or more sgRNAs targeting exon 11 of fTNFRSF11A.

### VI. Delivery

### A. Viral Vectors

In one aspect, the present disclosure encompasses methods of delivery of a CRISPR gene-editing system targeting a gene selected from IL1R1, IL1R2, IL1RAP, IL1RL1, TGFBR1, TGFBR2, IL6R, IL6ST, TNFRSF1A, TNFRSF1B, TNFRSF3, TNFRSF4, TNFRSF11A and combinations thereof using one or more recombinant viral particle. In some embodiments, the one of more viral vectors comprise a recombinant virus selected from a retrovirus, an adenovirus, an adeno-associated virus, a lentivirus, and a herpes simplex virus-1. In some embodiments, the one of more viral vectors comprise a recombinant adeno-associated virus (AAV). In some embodiments, the recombinant AAV is of serotype 5 (AAV5). In some embodiments, the recombinant AAV is of serotype 6 (AAV6). In some embodiments, the one or more viral vectors comprise: a first viral vector comprising a first nucleic acid, in the one or more nucleic acids, encoding the Cas protein; and a second viral vector comprising a second nucleic acid, in the one or more nucleic acids, encoding the at least one guide RNA. In some embodiments, the one or more viral vectors comprise a viral vector comprising a single nucleic acid, wherein the single nucleic acid encodes the Cas9 protein and the at least one guide RNA.

### 1. Adeno-Associated Virus (AAV)

A viral vector system useful for delivery of nucleic acids is the adeno-associated virus (AAV). Adeno-associated virus is a naturally occurring defective virus that requires another virus, such as an adenovirus or a herpes virus, as a helper virus for efficient replication and a productive life cycle. For a review see Muzyczka et al., Curr. Topics in Micro. and Immunol. 158:97-129 (1992). It is also one of the few viruses that may integrate its DNA into non-dividing cells, and exhibits a high frequency of stable integration (see for example Flotte et al., Am. J. Respir. Cell. Mol. Biol. 7:349-356 (1992); Samulski et al., J. Virol. 63:3822-3828 (1989); and Mclaughlin et al., J. Virol. 62:1963-1973 (1989). Vectors containing as little as 300 base pairs of AAV can be packaged and can integrate. Space for exogenous DNA is limited to about 4.5 kb. An AAV vector such as that described in Tratschin et al., Mol. Cell. Biol. 5:3251-3260 (1985) can be used to introduce DNA into cells. A variety of nucleic acids have been introduced into different cell types using AAV vectors (see for example Hermonat et al., Proc. Natl. Acad. Sci. USA 81:6466-6470 (1984); Tratschin et al., Mol. Cell. Biol. 4:2072-2081 (1985); Wondisford et al., Mol. Endocrinol. 2:32-39 (1988); Tratschin et al., J. Virol. 51:611-619 (1984); and Flotte et al., J. Biol. Chem. 268:3781-3790 (1993). The identification of Staphylococcus aureus (SaCas9) and other smaller Cas9 enzymes that can be packaged into adeno-associated viral (AAV) vectors that are highly stable and effective in vivo, easily produced, approved by FDA, and tested in multiple clinical trials, paves new avenues for therapeutic gene editing.

According to particular embodiments, a CRISPR gene-editing system targeting a gene selected from IL1R1, IL1R2, IL1RAP, IL1RL1, TGFBR1, TGFBR2, IL6R, IL6ST, TNFRSF1A, TNFRSF1B, TNFRSF3, TNFRSF4, TNFRSF11A, and combinations thereof further comprise a recombinant AAV vector. In some embodiments, the CRISPR gene-editing system is encoded by a nucleic acid, wherein the nucleic acid is a recombinant AAV genome. In some embodiments, the AAV vector is selected from an AAV1 vector, an AAV2 vector, an AAV3 vector, an AAV4 vector, an AAV5 vector, an AAV6 vector, an AAV7 vector, an AAV8 vector, an AAV9 vector, and an AAV10 vector.

In some aspects, the AAV vector comprises a serotype selected from the group consisting of: AAV1, AAV1 (Y705+731F+T492V), AAV2 (Y444+500+730F+T491V), AAV3 (Y705+731F), AAV4, AAV5, AAV5 (Y436+693+719F), AAV6, AAV6 (VP3 variant Y705F/Y731F/T492V), AAV-7m8, AAV8, AAV8 (Y733F), AAV9, AAV9 (VP3 variant Y731F), AAV10 (Y733F), AAV-ShH10, and AAV-DJ/8. In some aspects, the AAV vector comprises a serotype selected from the group consisting of: AAV1, AAV5, AAV6, AAV6 (Y705F/Y731F/T492V), AAV8, AAV9, and AAV9 (Y731F).

In some embodiments, use of the CRISPR gene-editing system further comprising one or more LNPs to target a gene selected from IL1R1, IL1RAP, TGFBR1, TGFBR2, IL6R, IL6ST, TNFRSF1A, TNFRSF1B, TNFRSF3, TNFRSF4, TNFRSF11A, and combinations thereof is therapeutic.

In one aspect, use of the CRISPR gene-editing system further comprising one or more AAV vectors to target TGFBR1 and/or TGFBR2 is therapeutic. In some embodiments, use of the system treats one or more joint disease or illness or one or more back or spine conditions or disorders. In some embodiments, use of the system treats one or more musculoskeletal diseases, conditions, and illnesses, including, but not limited to, Loeys-Dietz Syndrome, osteoarthritis, Marfan syndrome, aortic aneurysm (e.g., familial thoracic 3 aortic aneurysm), craniofacial abnormalities, and combinations thereof. In other embodiments, use of the system treats neoplastic diseases, conditions, and illnesses, including, but not limited to, pancreatic cancer, multiple self-healing squamous epithelioma (Ferguson-Smith disease), gastrointestinal stromal tumors (GIST), hereditary nonpolyposis colorectal cancer (Lynch Syndrome), metastatic colorectal carcinoma, bone neoplasms, anaplastic carcinoma, spindle-cell carcinoma, lung neoplasms, brain neoplasms, and combinations thereof.

In some embodiments, the CRISPR gene-editing system further comprising one or more AAV vectors to target IL1R1 and/or IL1RAP is therapeutic. In some embodiments, use of the system treats one or more joint disease or illness or one or more back or spine conditions or disorders. In some embodiments, use of the system treats one or more musculoskeletal diseases, conditions, and illnesses, including, but not limited to, rheumatoid arthritis, gout, osteoarthritis, immune-mediated arthritis or polyarthritis, osteoporosis, intervertebral disc disease (IVDD), psoriatic arthritis, arthritis, polymyositis, acute or chronic inflammatory synovitis, proliferative synovitis, bone neoplasms, sarcoid myopathy, cortex bone disorders, idiopathic scoliosis, tendinopathy, myofibrillar myopathy, enthesis-related arthritis, ankylosing spondylitis, degenerative polyarthritis, arthopathy, osteitis deformans, prolapsed lumbar disc, polymyositis ossificans, idiopathic polymyositis, Luft Disease, adult-onset Still's Disease, osteoarthritis deformans, Bachet's Disease, and combinations thereof. In other embodiments, use of the system treats one or more neoplastic diseases, conditions, and illnesses, including, but not limited to, osteosarcoma, multiple myeloma, lymphoma (e.g., B-cell or cutaneous T-cell lymphoma), leukemia, thyroid carcinoma, glioma, renal cell carcinoma, chondrosarcoma, glioblastoma, melanoma, neuroblastoma, polycystic ovary syndrome, Kaposi sarcoma, squamous cell carcinoma, leiomyosarcoma, malignant peripheral nerve sheath tumor, Ewing's sarcoma, esophageal neoplasms, colon cancer, lung cancer, breast cancer, pancreatic cancer, stomach cancer, epithelial ovarian cancer, cholectoral cancer, liver cancer, skin cancer, prostate cancer, cervical cancer, ovarian cancer, bladder cancer, oral cavity cancer, and combinations thereof. In other embodiments, use of the system treats one or more inflammatory diseases, conditions, and illnesses, including, but not limited to, autoinflammatory disease (AID), cryopyrin-associated periodic syndrome (CAPS), systemic lupus erythematosus (SLE), inflammatory bowel disease (IBD) and combinations thereof. In some embodiments, use of the system treats one or more cardiac diseases, conditions, and illnesses, including, but not limited to, myocardial infarction, heart failure (e.g., refractory or acute decompensated heart failure), arrthymias, pericarditis (e.g., refractory idiopathic pericarditis), myocarditis, sepsis-induced cardiomyopathy, atherosclerosis, coronary artery disease and combinations thereof. In some embodiments, use of the system treats one or more neurological diseases, conditions, and illnesses, including, but not limited to, acute thrombotic stroke, epilepsy, multiple sclerosis, Alzheimer's Disease and combinations thereof. In some embodiments, use of the system treats one or more opthalmic diseases, conditions, and illnesses, including, but not limited to, uveitis, scleritis, Sjogren asthenia, dry eye and combinations thereof. In some embodiments, use of the system treats one or more diseases, conditions, and illnesses, including, but not limited to, chronic kidney disease, Type 2 diabetes, gastroesophageal reflux disease (GERD), non-HP-associated peptic ulcer disease, and pulmonary fibrosis.

### 2. Lentivirus

In some aspects, the viral vector is a lentivirus. In an aspect, the lentivirus is selected from the group consisting of: human immunodeficiency-1 (HIV-1), human immunodeficiency-2 (HIV-2), simian immunodeficiency virus (SIV), feline immunodeficiency virus (FIV), bovine immunodeficiency virus (BIV), Jembrana Disease Virus (JDV), equine infectious anemia virus (EIAV), and caprine arthritis encephalitis virus (CAEV).

Lentiviral transduction systems are known in the art and are described, e.g., in Levine, et al., Proc. Nat'l Acad. Sci. 2006, 103, 17372-77; Zufferey, et al., Nat. Biotechnol. 1997, 15, 871-75; Dull, et al., J. Virology 1998, 72, 8463-71, and U.S. Pat. No. 6,627,442, the disclosures of each of which are incorporated by reference herein.

In one aspect, use of the CRISPR gene-editing system further comprising one or more lentiviral vectors to target TGFBR1 and/or TGFBR2 is therapeutic. In some embodiments, use of the system treats one or more joint disease or illness, fibrosis and/or scarring, or one or more back or spine conditions or disorders. In some embodiments, use of the system treats one or more musculoskeletal diseases, conditions, and illnesses, including, but not limited to, Loeys-Dietz Syndrome, osteoarthritis, Marfan syndrome, aortic aneurysm (e.g., familial thoracic 3 aortic aneurysm), craniofacial abnormalities, and combinations thereof. In other embodiments, use of the system treats neoplastic diseases, conditions, and illnesses, including, but not limited to, pancreatic cancer, multiple self-healing squamous epithelioma (Ferguson-Smith disease), gastrointestinal stromal tumors (GIST), hereditary nonpolyposis colorectal cancer (Lynch Syndrome), metastatic colorectal carcinoma, bone neoplasms, anaplastic carcinoma, spindle-cell carcinoma, lung neoplasms, brain neoplasms, and combinations thereof.

In some embodiments, the CRISPR gene-editing system further comprising one or more lentiviral vectors to target IL1R1 and/or IL1RAP is therapeutic. In some embodiments, use of the system treats one or more joint disease or illness, fibrosis and/or scarring, or one or more back or spine conditions or disorders. In some embodiments, use of the system treats one or more musculoskeletal diseases, conditions, and illnesses, including, but not limited to, rheumatoid arthritis, gout, osteoarthritis, osteoporosis, intervertebral disc disease (IVDD), psoriatic arthritis, arthritis, polymyositis, proliferative synovitis, bone neoplasms, sarcoid myopathy, cortex bone disorders, idiopathic scoliosis, tendinopathy, myofibrillar myopathy, enthesis-related arthritis, ankylosing spondylitis, degenerative polyarthritis, arthropathy, osteitis deformans, prolapsed lumbar disc, polymyositis ossificans, idiopathic polymyositis, Luft Disease, adult-onset Still's Disease, osteoarthritis deformans, Bachet's Disease, and combinations thereof. In other embodiments, use of the system treats one or more neoplastic diseases, conditions, and illnesses, including, but not limited to, osteosarcoma, multiple myeloma, lymphoma (e.g., B-cell or cutaneous T-cell lymphoma), leukemia, thyroid carcinoma, glioma, renal cell carcinoma, chondrosarcoma, glioblastoma, melanoma, neuroblastoma, polycystic ovary syndrome, Kaposi sarcoma, squamous cell carcinoma, leiomyosarcoma, malignant peripheral nerve sheath tumor, Ewing's sarcoma, esophageal neoplasms, colon cancer, lung cancer, breast cancer, pancreatic cancer, stomach cancer, epithelial ovarian cancer, cholectoral cancer, liver cancer, skin cancer, prostate cancer, cervical cancer, ovarian cancer, bladder cancer, oral cavity cancer, and combinations thereof. In other embodiments, use of the system treats one or more inflammatory diseases, conditions, and illnesses, including, but not limited to, autoinflammatory disease (AID), cryopyrin-associated periodic syndrome (CAPS), systemic lupus erythematosus (SLE), inflammatory bowel disease (IBD) and combinations thereof. In some embodiments, use of the system treats one or more cardiac diseases, conditions, and illnesses, including, but not limited to, myocardial infarction, heart failure (e.g., refractory or acute decompensated heart failure), arrythmias, pericarditis (e.g., refractory idiopathic pericarditis), myocarditis, sepsis-induced cardiomyopathy, atherosclerosis, coronary artery disease and combinations thereof. In some embodiments, use of the system treats one or more neurological diseases, conditions, and illnesses, including, but not limited to, acute thrombotic stroke, epilepsy, multiple sclerosis, Alzheimer's Disease and combinations thereof. In some embodiments, use of the system treats one or more ophthalmic diseases, conditions, and illnesses, including, but not limited to, uveitis, scleritis, Sjogren asthenia, dry eye and combinations thereof. In some embodiments, use of the system treats one or more diseases, conditions, and illnesses, including, but not limited to, chronic kidney disease, Type 2 diabetes, gastroesophageal reflux disease (GERD), non-HP-associated peptic ulcer disease, and pulmonary fibrosis.

### B. Lipid Nanoparticles (LNP)

In some embodiments, a CRISPR gene-editing system is delivered by a nanoparticle. Without wishing to be bound by any particular theory, in certain embodiments, nucleic acids, when present in the nanoparticle, are resistant in aqueous solution to degradation with a nuclease. In other embodiments, proteins are protected from protease degradation. In some embodiments, proteins and nucleic acids encapsulated by nanoparticles are capable of penetrating the cellular plasma membrane.

Lipid nanoparticles comprising nucleic acids and their method of preparation is disclosed in at least WO2017/019935, WO2017/049074, WO2017/201346, WO2017/218704, WO2018/006052, WO2018/013525, WO2018/089540, WO2018/119115, WO2018/126084, WO2018/157009, WO2018/170336, WO2018/222890, WO2019/046809, WO2019/089828, WO2020/061284, WO2020/061317, WO2020/081938, WO2020/097511, WO2020/097520, WO2020/097540, WO2020/097548, WO2020/214946, WO2020/219941, WO2020/232276, WO2020/227615, WO2020/061295, WO2021/007278, WO2021/016430, WO2021/021988, EP Patent No. EP 2 972 360, US20200155691, US20200237671, U.S. Pat. Nos. 8,058,069, 8,492,359, 8,822,668, 9,364,435, 9,404,127, 9,504,651, 9,593,077, 9,738,593, 9,868,691, 9,868,692, 9,950,068, 10,138,213, 10,166,298, 10,221,127, 10,238,754, 10,266,485, 10,383,952, 10,730,924, 10,766,852, 11,079,379, 11,141,378 and 11,246,933, which are incorporated herein by reference in their entirety for all purposes.

### Lipid Nanoparticle Compositions

In some embodiments, the largest dimension of a nanoparticle composition is 1 micrometer or shorter (e.g., 1 micrometer, 900 nm, 800 nm, 700 nm, 600 nm, 500 nm, 400 nm, 300 nm, 200 nm, 175 nm, 150 nm, 125 nm, 100 nm, 75 nm, 50 nm, or shorter), e.g., when measured by dynamic light scattering (DLS), transmission electron microscopy, scanning electron microscopy, or another method. Nanoparticle compositions include, for example, lipid nanoparticles (LNPs), liposomes, lipid vesicles, and lipoplexes. In some embodiments, nanoparticle compositions are vesicles including one or more lipid bilayers. In certain embodiments, a nanoparticle composition includes two or more concentric bilayers separated by aqueous compartments. Lipid bilayers may be functionalized and/or crosslinked to one another. Lipid bilayers may include one or more ligands, proteins, or channels. In various embodiments, lipid nanoparticles described herein have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 nm to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm, and are substantially non-toxic.

In certain embodiments, the lipid nanoparticles described herein comprise one or more components, including a lipid component, and (optionally) a structural component. The lipid component comprises lipids selected from ionizable and/or cationic lipids (i.e., lipids that may have a positive or partial positive charge at physiological pH), neutral lipids (e.g., phospholipids, or sphingolipids), and polymer-conjugated lipids (e.g., PEGylated lipids). In some embodiments, the lipid component comprises a single ionizable lipid. In other embodiments, the lipid component comprises 2, 3, 4, 5, 6, 7, 8, 9, 10, 15, or 20 ionizable lipids. In some embodiments, the lipid component comprises a single neutral lipid. In other embodiments, the lipid component comprises 2, 3, 4, 5, 6, 7, 8, 9, 10, 15, or 20 neutral lipids. In some embodiments, the lipid component comprises a single polymer-conjugated lipid. In other embodiments, the lipid component comprises 2, 3, 4, 5, 6, 7, 8, 9, 10, 15, or 20 polymer-conjugated lipids. In some embodiments, the structural component comprises a single structural lipid. In other embodiments, the structural component comprises 2, 3, 4, 5, 6, 7, 8, 9, 10, 15, or 20 structural lipids. In some embodiments, the lipid component comprises at least one cationic lipid, at least one neutral lipid, and at least one polymer-conjugated lipid. The present disclosure contemplates that the lipid component may comprise any combination of the foregoing constituents.

### Ionizable/Cationic Lipids

In some embodiments, the lipid component comprises an ionizable lipid. In some embodiments, the ionizable lipid is anionic. In other embodiments, the ionizable lipid is a cationic lipid. In some embodiments, the lipid component comprises cationic lipids including, but not limited to, a cationic lipid selected from the group consisting of 3-(didodecylamino)-N1,N1,4-tridodecyl-1-piperazineethanamine (KL10), N1-[2-(didodecylamino)ethyl]-N1,N4,N4-tridodecyl-1,4-piperazinediethanamine (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl 4-(dimethylamino) butanoate (DLin-MC3-DMA), 2,2-dilinoleyl-4-(2-dimethylaminoethyl)-[1,3]-dioxolane (DLin-KC2-DMA), 1,2-dioleyloxy-N,N-dimethylaminopropane (DODMA), 2-({8-[(3.beta.)-cholest-5-en-3-yloxy]octyl}oxy)-N,N-dimethyl-3-[(9Z,12Z)--octadeca-9,12-dien-1-yloxy]propan-1-amine (Octyl-CLinDMA), (2R)-2-({8-[(3.beta.)-cholest-5-en-3-yloxy]octyl}oxy)-N,N-dimethyl-3-[(9Z-,12Z)-octadeca-9,12-dien-1-yloxy]propan-1-amine (Octyl-CLinDMA (2R)), (2S)-2-({8-[(3.beta.)-cholest-5-en-3-yloxy]octyl}oxy)-N,N-dimethyl-3-[(9Z-,12Z)-octadeca-9,12-dien-1-yloxy]propan-1-amine (Octyl-CLinDMA (2S)), a lipid including a cyclic amine group, and mixtures thereof.

Non-exhaustive and non-limiting examples of cationic lipids include:

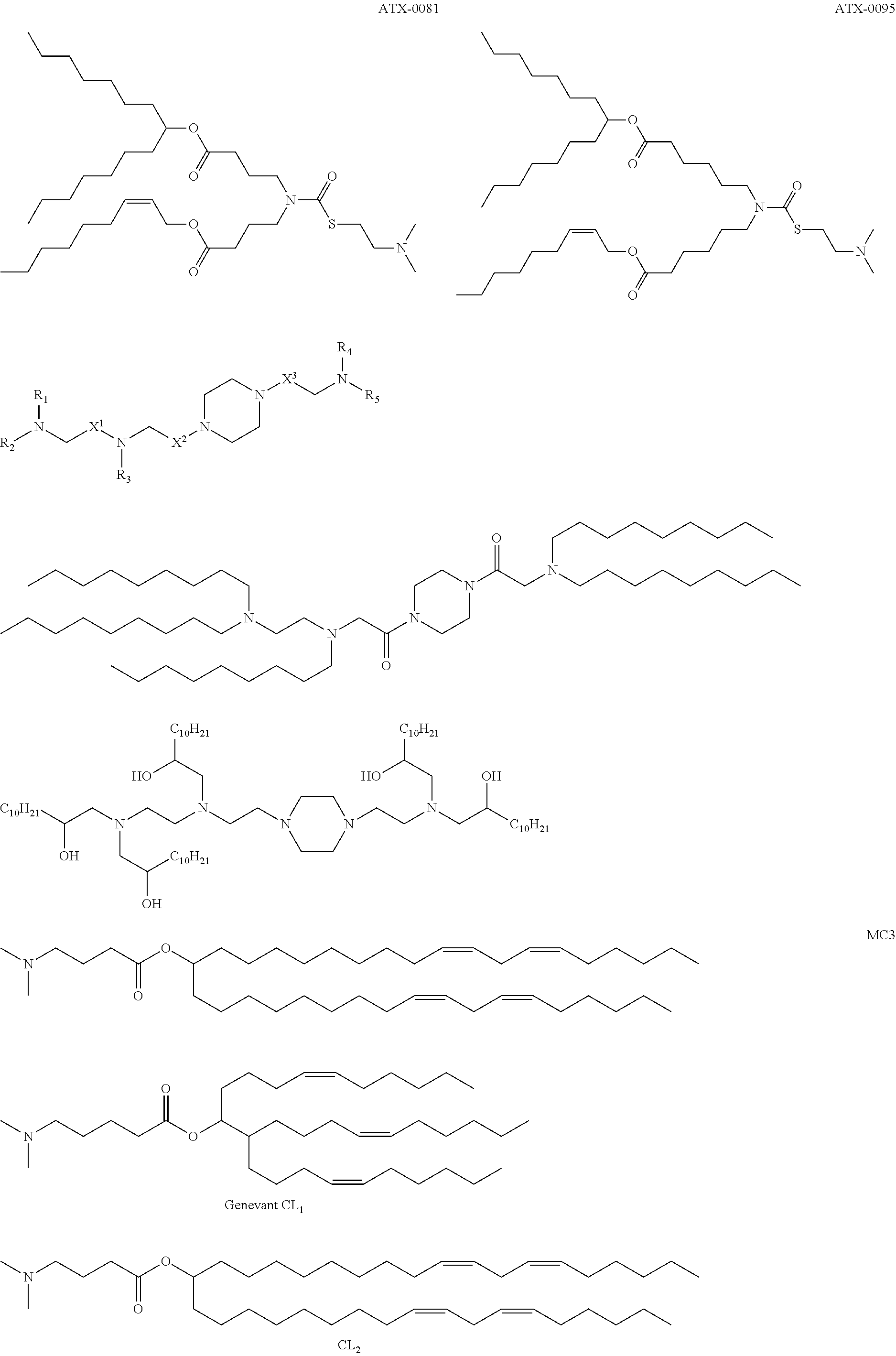

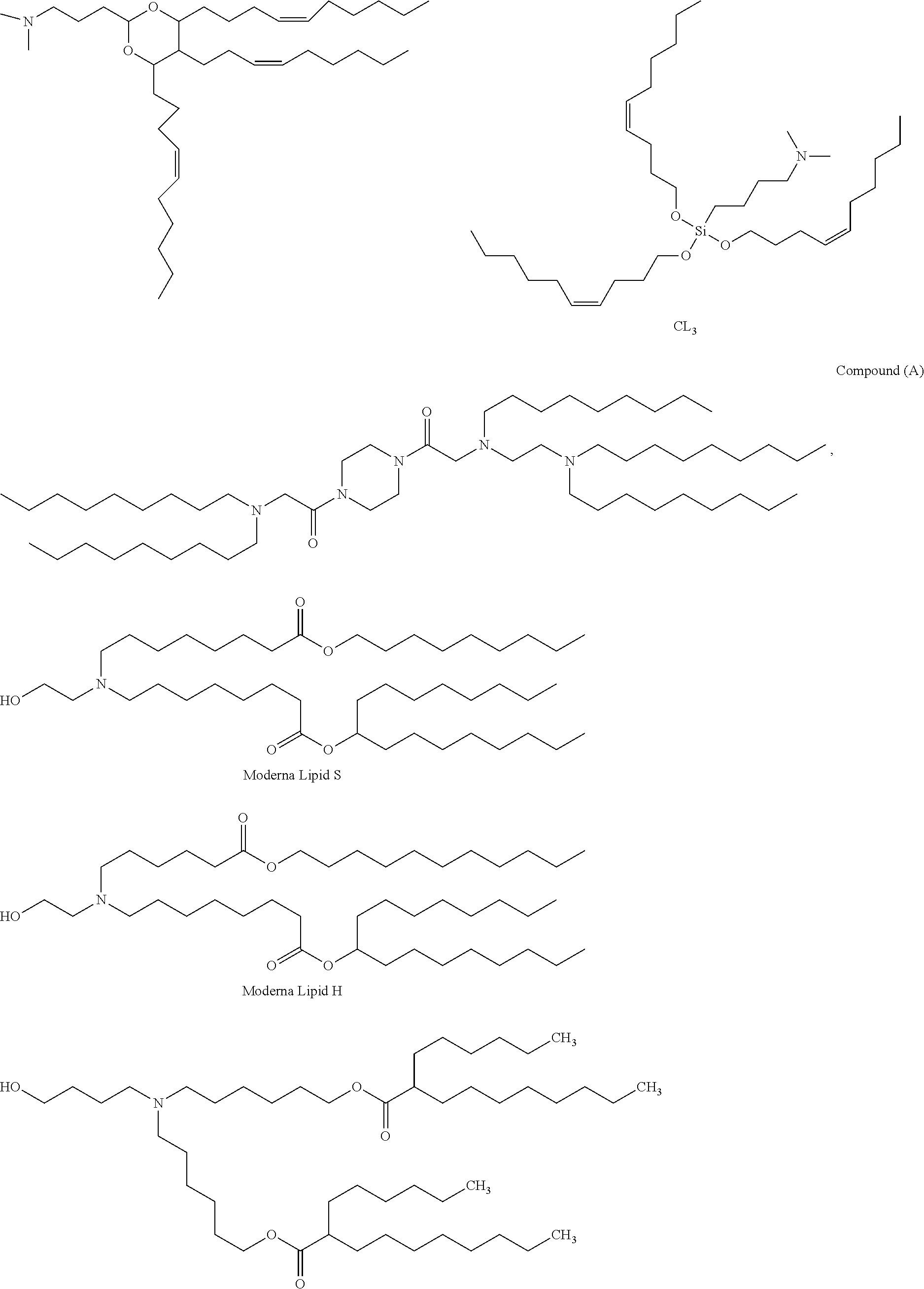 [SMILES: CCCCC/C=C\CCCC1OC(CCCN(C)C)OC(CCC/C=C\CCCCC)C1CC/C=C\CCCCC ; CCCCC/C=C\CCCO[Si](CCCCN(C)C)(OCCC/C=C\CCCCC)OCCC/C=C\CCCCC ; CCCCCCCCCN(CCCCCCCCC)CCN(CCCCCCCCC)CC(=O)N1CCN(C(=O)CN(CCCCCCCCC)CCCCCCCCC)CC1 ; CCCCCCCCCOC(=O)CCCCCCCN(CCO)CCCCCCCC(=O)OC(CCCCCCCC)CCCCCCCC ; CCCCCCCCCCCOC(=O)CCCCCN(CCO)CCCCCCCC(=O)OC(CCCCCCCC)CCCCCCCC ; CCCCCCCCC(CCCCCC)C(=O)OCCCCCCN(CCCCO)CCCCCCOC(=O)C(CCCCCC)CCCCCCCC]

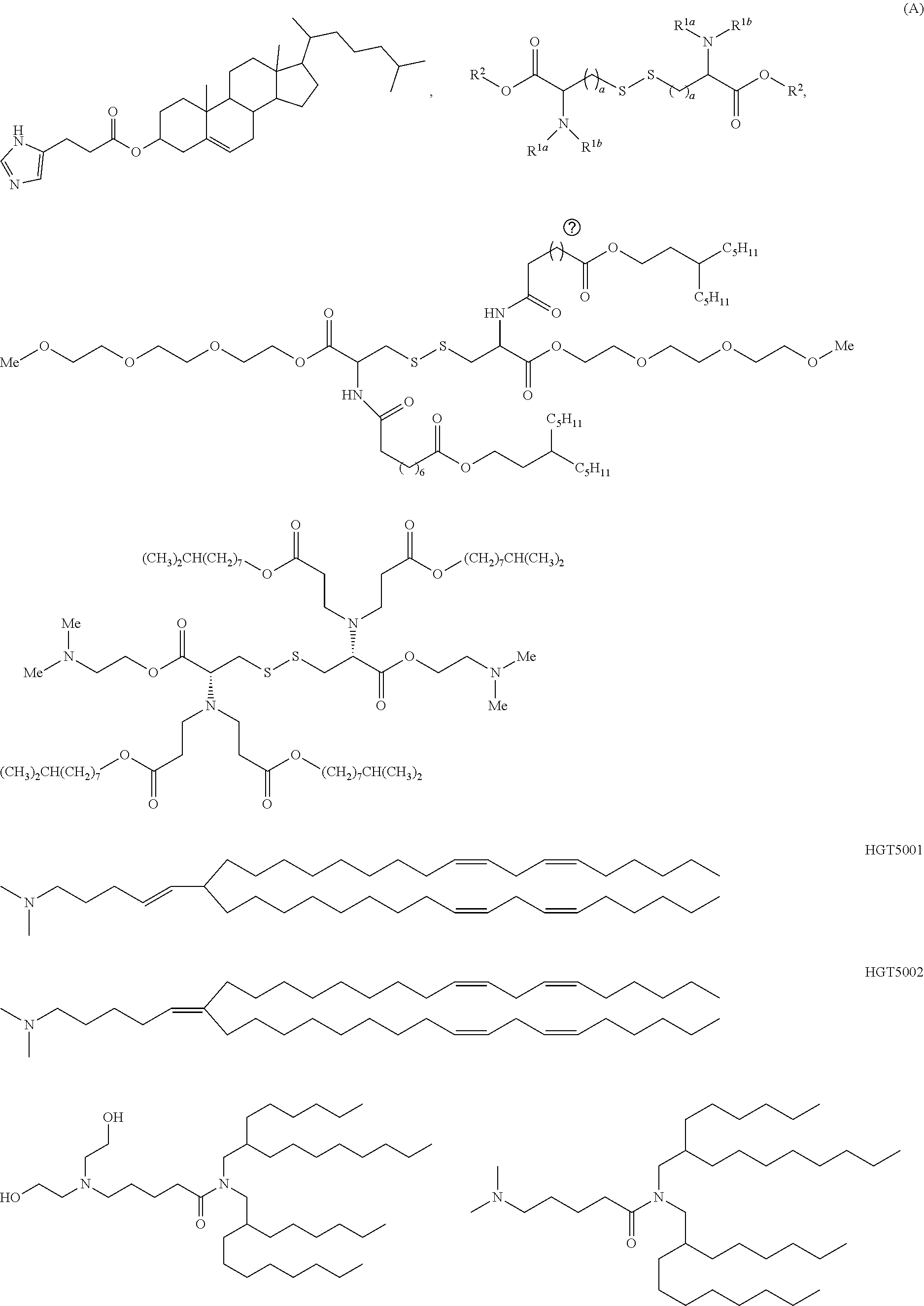

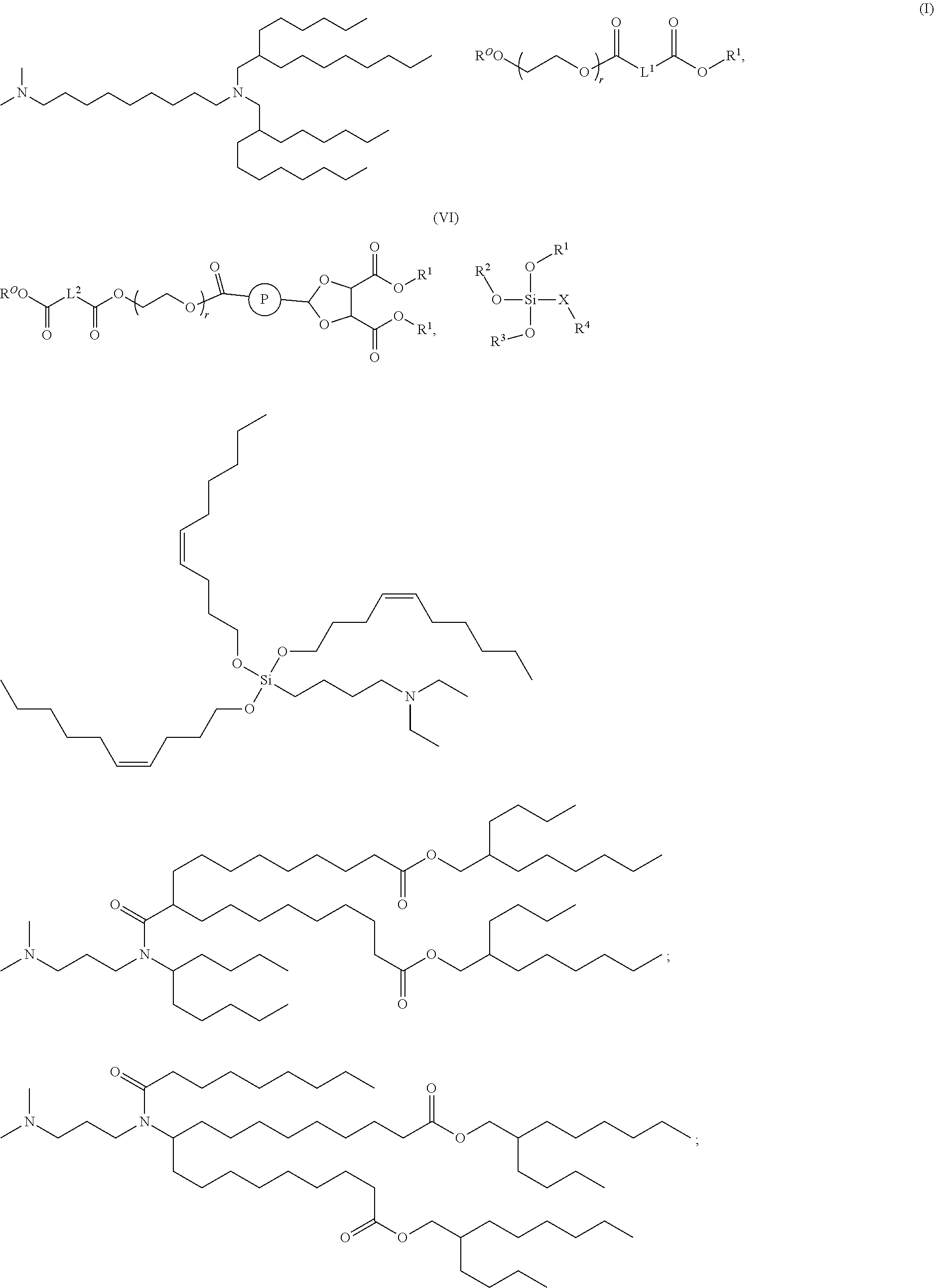

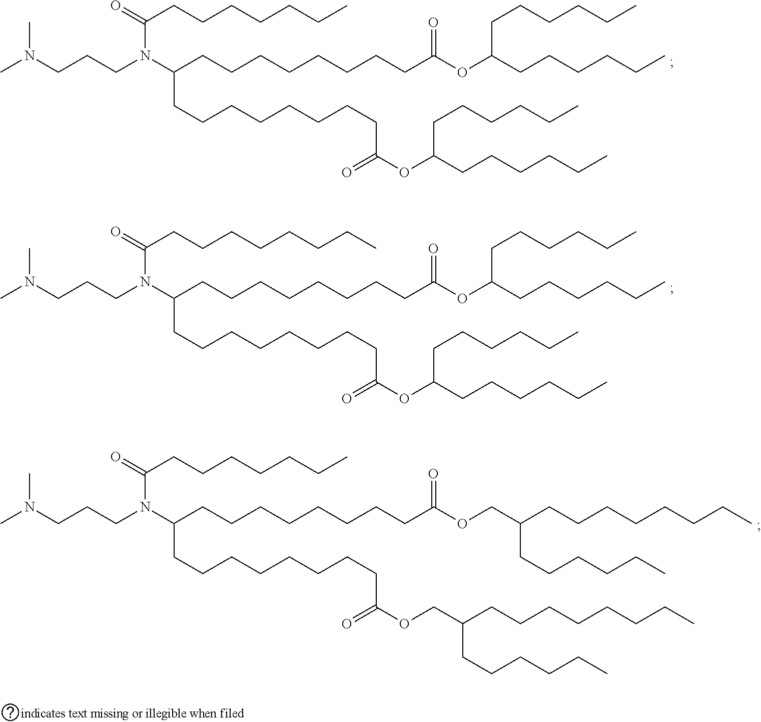

### Neutral Lipids/Phospholipids

In some embodiments, the lipid component further comprises neutral lipids including, but not limited to, a phospholipid selected from the group consisting of 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanolamine (DOPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin (SM), and mixtures thereof.

### Polymer-Conjugated Lipids

In some embodiments, the lipid component further comprises polymer-conjugated lipids, including, but not limited to, a PEGylated lipid selected from the group consisting of PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG_{2000-c}-DMG, PEG-DLPE, PEG-DMPE, PEG-DPPC, PEG-DMA or a PEG-DSPE lipid.

### Non-Exhaustive and Non-Limiting Examples of PEG Lipids Include:

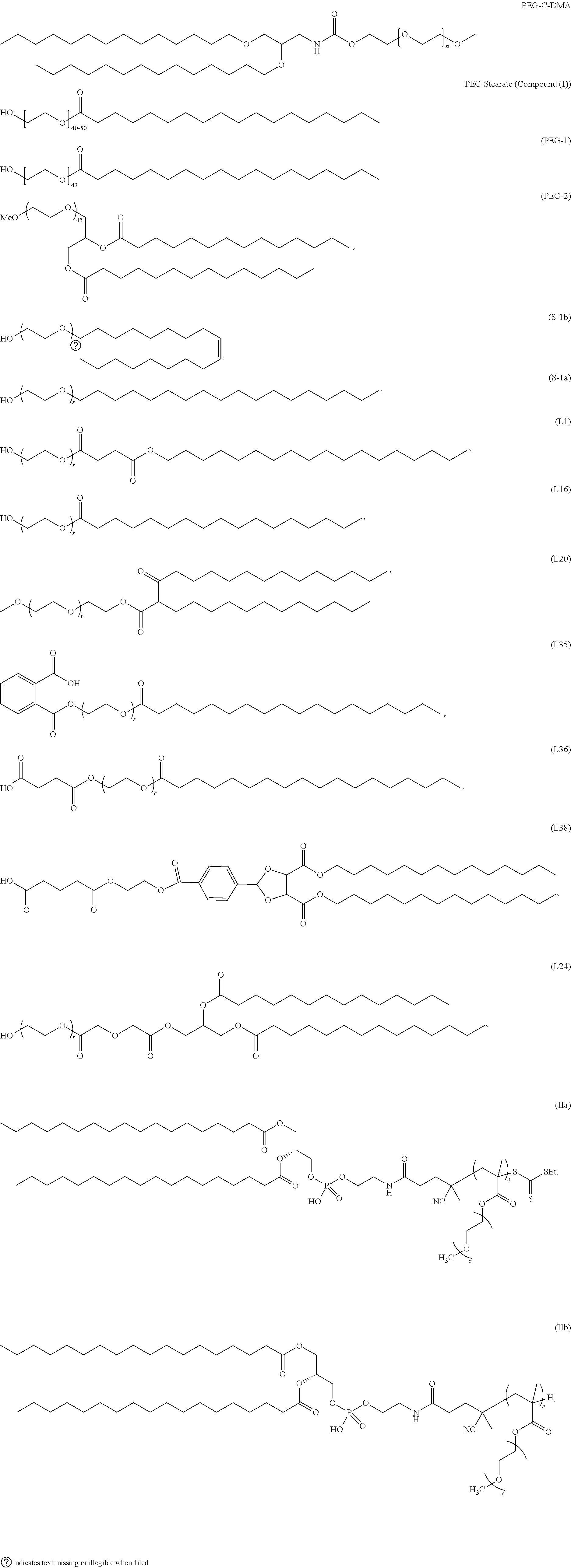

### Structural Lipids/Sterols

In some embodiments, the LNP further comprises a structural component. See generally Patel, S., et al. (2020). Nature Communications, 11 (1), 1-13. In some embodiments, the structural component comprises a sterol including, but not limited to, a sterol selected from the group consisting of cholesterol, fecosterol, stigmasterol, stigmastanol, sitosterol, β-sitosterol, lupeol, betulin, ursolic acid, oleanolic acid, campesterol, fucosterol, brassicasterol, ergosterol, 9,11-dehydroergosterol, tomatidine, tomatine, α-tocopherol, and mixtures thereof. In other embodiments, the structural lipid includes cholesterol and a corticosteroid (e.g., prednisolone, dexamethasone, prednisone, and hydrocortisone), or a combination thereof.

### Non-Exhaustive and Non-Limiting Examples of Structural Lipids Include:

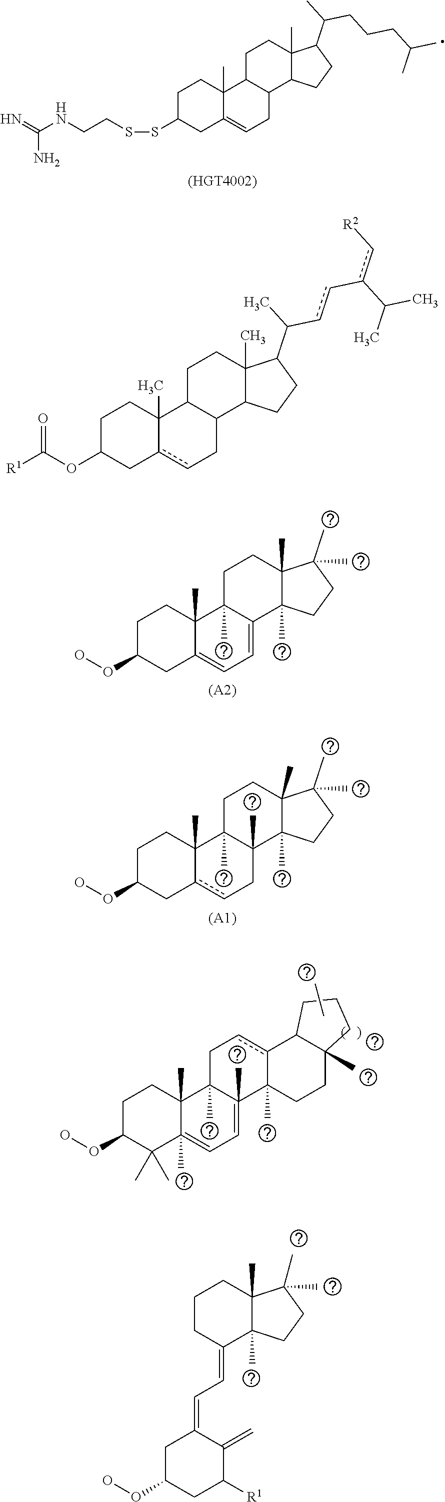

### Formulations

Nanoparticle compositions may include a lipid component and one or more additional components, such as a therapeutic and/or prophylactic. A nanoparticle composition may be designed for one or more specific applications or targets. The elements of a nanoparticle composition may be selected based on a particular application or target, and/or based on the efficacy, toxicity, expense, ease of use, availability, or other feature of one or more elements. Similarly, the particular formulation of a nanoparticle composition may be selected for a particular application or target according to, for example, the efficacy and toxicity of particular combinations of elements.

The lipid component of a nanoparticle composition may include, for example, a cationic lipid, a phospholipid (such as an unsaturated lipid, e.g., DOPE or DSPC), a PEG lipid, and a structural lipid. The elements of the lipid component may be provided in specific fractions.

In some embodiments, the lipid component of a nanoparticle composition includes an ionizable lipid, a phospholipid, a PEG lipid, and a structural lipid. In certain embodiments, the lipid component of the nanoparticle composition includes about 30 mol % to about 60 mol % ionizable lipid, about 0 mol % to about 30 mol % phospholipid, about 0 mol % to about 10 mol % of PEG lipid, and about 17.5 mol % to about 50 mol % structural lipid, provided that the total mol % does not exceed 100%. In some embodiments, the lipid component of the nanoparticle composition includes about 35 mol % to about 55 mol % compound of ionizable lipid, about 5 mol % to about 25 mol % phospholipid, about 0 mol % to about 10 mol % of PEG lipid, and about 30 mol % to about 40 mol % structural lipid. In a particular embodiment, the lipid component includes about 50 mol % said compound, about 10 mol % phospholipid, about 38.5 mol % structural lipid, and about 1.5 mol % of PEG lipid. In another embodiment, the lipid component includes about 40 mol % said compound, about 20 mol % phospholipid, about 38.5 mol % structural lipid, and about 1.5 mol % of PEG lipid. In some embodiments, the phospholipid may be DOPE or DSPC. In other embodiments, the PEG lipid may be PEG-DMG and/or the structural lipid may be cholesterol.

In some embodiments, the ionizable lipids comprise between about 20 and about 60 mol % of the lipid component. In other embodiments, the ionizable lipids comprise between about 35 and about 55 mol % of the lipid component. In various embodiments, the ionizable lipids comprise about 20, 21, 22, 23, 24, 25, 26, 27, 28, 29, 30, 35, 40, 45, 50, 55, or 60 mol % of the lipid component.

In some embodiments, the neutral lipids comprise between about 0 and about 30 mol % of the lipid component. In other embodiments, the neutral lipids comprise between about 5 and about 25 mol % of the lipid component. In various embodiments, the neutral lipids comprise about 1, 2, 3, 4, 5, 6, 7, 8, 9, 10, 11, 12, 13, 14, 15, 16, 17, 18, 19, 20, 25, or 30 mol % of the lipid component.

In some embodiments, the polymer-conjugated lipids comprise between about 0 and about 15 mol % of the lipid component. In other embodiments, the polymer-conjugated lipids comprise between about 0.5 and about 10 mol % of the lipid component. In various embodiments, the polymer-conjugated lipids comprise about 0.5, 1, 1.5, 2, 2.5, 3, 3.5, 4, 4.5, 5, 5.5, 6, 6.5, 7, 7.5, 8, 8.5 9, 9.5, 10, or 15 mol % of the lipid component.

In some embodiments, the structural component comprises about 17.5 mol % to about 50 mol % of the lipid component. In other embodiments, the structural component comprises about 30 to about 40 mol % of the lipid component. In various embodiments, the structural component comprises about 17.5, 20, 22.5, 25, 27.5, 30, 31, 32, 33, 34, 35, 36, 37, 38, 39, 40, 41, 42, 43, 44, 45, 46, 47, 48, 49, or 50 mol % of the lipid component.

The structural component may alternatively be expressed as a ratio relative to the lipid component. In some embodiments, the structural component is in a ratio of about 1:1 with the lipid component (sterol: lipids). In other embodiments, the structural component is in a ratio of about 1:5 with the lipid component (sterol: lipids). In various embodiments, the structural component is in a ratio of about 1:1, 1:2, 1:3, 1:4, 1:5, 1:6, 1:7, 1:8, 1:9, 1:10, 1:15, 1:20, or 1:25 with the lipid component (sterol: lipids).

Nanoparticle compositions may be designed for one or more specific applications or targets. For example, a nanoparticle composition may be designed to deliver a therapeutic and/or prophylactic such as an RNA to a particular cell, tissue, organ, or system or group thereof in a mammal's body. Physiochemical properties of nanoparticle compositions may be altered in order to increase selectivity for particular bodily targets. For instance, particle sizes may be adjusted based on the fenestration sizes of different organs. The therapeutic and/or prophylactic included in a nanoparticle composition may also be selected based on the desired delivery target or tar-gets. For example, a therapeutic and/or prophylactic may be selected for a particular indication, condition, disease, or disorder and/or for delivery to a particular cell, tissue, organ, or system or group thereof (e.g., localized or specific delivery). In certain embodiments, a nanoparticle com-position may include an mRNA encoding a polypeptide of interest capable of being translated within a cell to produce the polypeptide of interest. Such a composition may be designed to be specifically delivered to a particular organ. In some embodiments, a composition may be de-signed to be specifically delivered to a mammalian joint.

The amount of a therapeutic and/or prophylactic in a nanoparticle composition may depend on the size, composition, desired target and/or application, or other properties of the nanoparticle composition as well as on the properties of the therapeutic and/or prophylactic. For example, the amount of an RNA useful in a nanoparticle composition may depend on the size, sequence, and other characteristics of the RNA. The relative amounts of a therapeutic and/or prophylactic and other elements (e.g., lipids) in a nanoparticle composition may also vary. In some embodiments, the wt/wt ratio of the lipid component to a therapeutic and/or prophylactic in a nanoparticle composition may be from about 5:1 to about 60:1, such as 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 45:1, 50:1, and 60:1. For example, the wt/wt ratio of the lipid component to a therapeutic and/or prophylactic may be from about 10:1 to about 40:1. In certain embodiments, the wt/wt ratio is about 20:1. The amount of a therapeutic and/or prophylactic in a nanoparticle composition may, for example, be measured using absorption spectroscopy (e.g., ultraviolet-visible spectroscopy).

In some embodiments, the therapeutic and/or prophylactic comprises a nucleic acid component. In some embodiments, the nucleic acid component comprises RNA including, but not limited to, RNA selected from the group consisting of messenger RNA (mRNA), CRISPR RNA (crRNA), tracrRNA, single-guide RNA (sgRNA), short interfering RNA (siRNA), antisense oligonucleotides (ASO), and mixtures thereof. In other embodiments, the nucleic acid component comprises DNA including, but not limited to, DNA selected from the group consisting of linear DNA, plasmid DNA, antisense oligonucleotide, and mixtures thereof.

In some embodiments, a nanoparticle composition includes one or more RNAs, and the one or more RNAs, lipids, and amounts thereof may be selected to provide a specific N:P ratio. The N:P ratio of the composition refers to the molar ratio of nitrogen atoms in one or more lipids to the number of phosphate groups in an RNA. In general, a lower N:P ratio is preferred. The one or more RNA, lipids, and amounts thereof may be selected to provide an N:P ratio from about 2:1 to about 30:1, such as 2:1, 3:1, 4:1, 5:1, 6:1, 7:1, 8:1, 9:1, 10:1, 12:1, 14:1, 16:1, 18:1, 20:1, 22:1, 24:1, 26:1, 28:1, or 30:1. In certain embodiments, the N:P ratio may be from about 2:1 to about 8:1. In other embodiments, the N:P ratio is from about 5:1 to about 8:1. For example, the N:P ratio may be about 5.0:1, about 5.5:1, about 5.67:1, about 6.0:1, about 6.5:1, or about 7.0:1. For example, the N:P ratio may be about 5.67:1.

In some embodiments, the nucleic acid component is comprised of a modified nucleic acid. For example, an RNA may be a modified RNA. That is, an RNA may include one or more nucleobases, nucleosides, nucleotides, or linkers that are non-naturally occurring. A “modified” species may also be referred to herein as an “altered” species. Species may be modified or altered chemically, structurally, or functionally. For example, a modified nucleobase species may include one or more substitutions that are not naturally occurring.

In certain embodiments, the present disclosure comprises methods for treating back or spine conditions or disorders. In other embodiments, the present disclosure comprises methods for treating discogenic disorders. In some embodiments, the present disclosure comprises methods for treating localized nociception, inflammation, or morphological changes associated with back or spine conditions or disorders in a subject in need thereof, the method comprising administering a therapeutically effective amount of a CRISPR-Cas composition encapsulated within or associated with a lipid nanoparticle (LNP), wherein the composition comprises one or more non-naturally occurring polynucleotides encoding a Cas9 protein and at least one sgRNA. In some embodiments, LNPs are administered intradiscally. In other embodiments, LNPs are administered epidurally. In some embodiments, LNPs are administered peridiscally. In some embodiments, LNPs are administered perivertebrally.

### Physical Properties

The characteristics of a nanoparticle composition may depend on the components thereof. For example, a nanoparticle composition including cholesterol as a structural lipid may have different characteristics than a nanoparticle composition that includes a different structural lipid. Similarly, the characteristics of a nanoparticle composition may depend on the absolute or relative amounts of its components. For instance, a nanoparticle composition including a higher molar fraction of a phospholipid may have different characteristics than a nanoparticle composition including a lower molar fraction of a phospholipid. Characteristics may also vary depending on the method and conditions of preparation of the nanoparticle composition.

Nanoparticle compositions may be characterized by a variety of methods. For example, microscopy (e.g., transmission electron microscopy or scanning electron microscopy) may be used to examine the morphology and size distribution of a nanoparticle composition. Dynamic light scattering or potentiometry (e.g., potentiometric titrations) may be used to measure zeta potentials. Dynamic light scattering may also be utilized to determine particle sizes. Instruments such as the Zetasizer Nano ZS (Malvern Instruments Ltd, Malvern, Worcestershire, UK) may also be used to measure multiple characteristics of a nanoparticle composition, such as particle size, polydispersity index, and zeta potential.

The mean size of a nanoparticle composition may be between 10 nm and 1 micrometer, e.g., measured by dynamic light scattering (DLS). For example, the mean size may be from about 40 nm to about 150 nm, such as about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In some embodiments, the mean size of a nanoparticle composition may be from about 50 nm to about 100 nm, from about 50 nm to about 90 nm, from about 50 nm to about 80 nm, from about 50 nm to about 70 nm, from about 50 nm to about 60 nm, from about 60 nm to about 100 nm, from about 60 nm to about 90 nm, from about 60 nm to about 80 nm, from about 60 nm to about 70 nm, from about 70 nm to about 100 nm, from about 70 nm to about 90 nm, from about 70 nm to about 80 nm, from about 80 nm to about 100 nm, from about 80 nm to about 90 nm, or from about 90 nm to about 100 nm. In certain embodiments, the mean size of a nanoparticle composition may be from about 70 nm to about 100 nm. In a particular embodiment, the mean size may be about 80 nm. In other embodiments, the mean size may be about 100 nm.

A nanoparticle composition may be relatively homogenous. A polydispersity index may be used to indicate the homogeneity of a nanoparticle composition, e.g., the particle size distribution of the nanoparticle compositions. A small (e.g., less than 0.3) polydispersity index generally indicates a narrow particle size distribution. A nanoparticle composition may have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of a nanoparticle composition may be from about 0.10 to about 0.20.

The zeta potential of a nanoparticle composition may be used to indicate the electrokinetic potential of the composition. For example, the zeta potential may describe the surface charge of a nanoparticle composition. Nanoparticle compositions with relatively low charges, positive or negative, are generally desirable, as more highly charged species may interact undesirably with cells, tissues, and other elements in the body. In some embodiments, the zeta potential of a nanoparticle composition may be from about −10 mV to about +20 mV, from about −10 mV to about +15 mV, from about −10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

The efficiency of encapsulation of a therapeutic and/or prophylactic describes the amount of therapeutic and/or prophylactic that is encapsulated or otherwise associated with a nanoparticle composition after preparation, relative to the initial amount provided. The encapsulation efficiency is desirably high (e.g., close to 100%). The encapsulation efficiency may be measured, for example, by comparing the amount of therapeutic and/or prophylactic in a solution containing the nanoparticle composition before and after breaking up the nanoparticle composition with one or more organic solvents or detergents. Fluorescence may be used to measure the amount of free therapeutic and/or prophylactic (e.g., RNA) in a solution. For the nanoparticle compositions described herein, the encapsulation efficiency of a therapeutic and/or prophylactic may be at least 50%, for example 50%, 55%, 60%, 65%, 70%, 75%, 80%, 85%, 9 0%, 91%, 92%, 93%, 94%, 95%, 96%, 97%, 98%, 99%, or 100%. In some embodiments, the encapsulation efficiency may be at least 80%. In certain embodiments, the encapsulation efficiency may be at least 90%.

A nanoparticle composition may optionally comprise one or more coatings. For example, a nanoparticle composition may be formulated in a capsule, film, or tablet having a coating. A capsule, film, or tablet including a composition described herein may have any useful size, tensile strength, hardness, or density.

Unless otherwise defined, all technical and scientific terms used herein have the same meaning as commonly understood by one of ordinary skill in the art to which this disclosure belongs. Although methods and materials similar or equivalent to those described herein may be used in the practice or testing of the present disclosure, suitable methods and materials are described below. All publications, patent applications, patents, and other references mentioned herein are incorporated by reference in their entirety. In addition, the materials, methods, and examples are illustrative only and not intended to be limiting.

In some embodiments, the CRISPR gene-editing system comprises one or more RNA-containing compositions. In some embodiments, the CRISPR gene-editing system further comprises one or more nanoparticles. In some embodiments, said one or more RNA-containing compositions comprises a guide RNA. In some embodiments, said one or more RNA-containing compositions comprises an mRNA. In some embodiments, said one or more RNA-containing compositions comprises an RNP (e.g., Cas9 and a guide RNA). In some embodiments, said one or more nanoparticles are lipid nanoparticles (LNP).

In some embodiments, the CRISPR gene-editing system comprises one or more LNPs collectively encapsulating (i) the RNA-guided nuclease or the nucleic acid encoding the RNA-guided nuclease and (ii) the at least one guide RNA or the nucleic acid encoding the at least one guide RNA. In some embodiments, the one or more LNPs comprises a first plurality of LNP encapsulating the RNA-guided nuclease or a nucleic acid encoding an RNA-guided nuclease and a second plurality of LNP encapsulating the at least one guide RNA or a nucleic acid encoding at least one guide RNA.

In some embodiments, the one or more LNP comprises a component selected from the group consisting of 3-(didodecylamino)-N1,N1,4-tridodecyl-1-piperazineethanamine (KL10), N1-[2-(didodecylamino)ethyl]-N1,N4,N4-tridodecyl-1,4-piperazinediethanamine (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl 4-(dimethylamino) butanoate (DLin-MC3-DMA), 2,2-dilinoleyl-4-(2-dimethylaminoethyl)-[1,3]-dioxolane (DLin-KC2-DMA), 1,2-dioleyloxy-N,N-dimethylaminopropane (DODMA), 2-({8-[(3.beta.)-cholest-5-en-3-yloxy]octyl}oxy)-N,N-dimethyl-3-[(9Z,12Z)--octadeca-9,12-dien-1-yloxy]propan-1-amine (Octyl-CLinDMA), (2R)-2-({8-[(3.beta.)-cholest-5-en-3-yloxy]octyl}oxy)-N,N-dimethyl-3-[(9Z-,12Z)-octadeca-9,12-dien-1-yloxy]propan-1-amine (Octyl-CLinDMA (2R)), (2S)-2-({8-[(3.beta.)-cholest-5-en-3-yloxy]octyl}oxy)-N,N-dimethyl-3-[(9Z-,12Z)-octadeca-9,12-dien-1-yloxy]propan-1-amine (Octyl-CLinDMA (2S)), a lipid including a cyclic amine group, and a mixture thereof.

In some embodiments, the one or more LNP comprises a component selected from the group consisting of 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanolamine (DOPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin (SM), and a mixture thereof.

In some embodiments, the one or more LNP comprises a component selected from the group consisting of PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG-DLPE, PEG-DMPE, PEG-DPPC, PEG-DMA, a PEG-DSPE lipid, and a mixture thereof.

In some embodiments, the one or more LNP comprises a component selected from the group consisting of a cholesterol, fecosterol, stigmasterol, stigmastanol, sitosterol, β-sitosterol, lupeol, betulin, ursolic acid, oleanolic acid, campesterol, fucosterol, brassicasterol, ergosterol, 9,11-dehydroergosterol, tomatidine, tomatine, α-tocopherol, and a mixture thereof.

In some embodiments, use of the CRISPR gene-editing system further comprising one or more LNPs to target a gene selected from IL1R1, IL1RAP, TGFBR1, TGFBR2, IL6R, IL6ST, TNFRSF1A, TNFRSF1B, TNFRSF3, TNFRSF4, TNFRSF11A, and combinations thereof is therapeutic.

In some aspect, use of the CRISPR gene-editing system further comprising one or more LNPs to target TGFBR1 and/or TGFBR2 is therapeutic. In some embodiments, use of the system treats one or more joint disease or illness or one or more back or spine conditions or disorders. In some embodiments, use of the system treats one or more musculoskeletal diseases, conditions, and illnesses, including, but not limited to, Loeys-Dietz Syndrome, osteoarthritis, Marfan syndrome, aortic aneurysm (e.g., familial thoracic 3 aortic aneurysm), craniofacial abnormalities, and combinations thereof. In other embodiments, use of the system treats neoplastic diseases, conditions, and illnesses, including, but not limited to, pancreatic cancer, multiple self-healing squamous epithelioma (Ferguson-Smith disease), gastrointestinal stromal tumors (GIST), hereditary nonpolyposis colorectal cancer (Lynch Syndrome), metastatic colorectal carcinoma, bone neoplasms, anaplastic carcinoma, spindle-cell carcinoma, lung neoplasms, brain neoplasms, and combinations thereof.

In some embodiments, the CRISPR gene-editing system further comprising one or more LNPs to target IL1R1 and/or IL1RAP is therapeutic. In some embodiments, use of the system treats one or more joint disease or illness or one or more back or spine conditions or disorders. In some embodiments, use of the system treats one or more musculoskeletal diseases, conditions, and illnesses, including, but not limited to, rheumatoid arthritis, gout, osteoarthritis, osteoporosis, intervertebral disc disease (IVDD), psoriatic arthritis, arthritis, polymyositis, proliferative synovitis, bone neoplasms, sarcoid myopathy, cortex bone disorders, idiopathic scoliosis, tendinopathy, myofibrillar myopathy, enthesis-related arthritis, ankylosing spondylitis, degenerative polyarthritis, arthropathy, osteitis deformans, prolapsed lumbar disc, polymyositis ossificans, idiopathic polymyositis, Luft Disease, adult-onset Still's Disease, osteoarthritis deformans, Bachet's Disease, and combinations thereof. In other embodiments, use of the system treats one or more neoplastic diseases, conditions, and illnesses, including, but not limited to, osteosarcoma, multiple myeloma, lymphoma (e.g., B-cell or cutaneous T-cell lymphoma), leukemia, thyroid carcinoma, glioma, renal cell carcinoma, chondrosarcoma, glioblastoma, melanoma, neuroblastoma, polycystic ovary syndrome, Kaposi sarcoma, squamous cell carcinoma, leiomyosarcoma, malignant peripheral nerve sheath tumor, Ewing's sarcoma, esophageal neoplasms, colon cancer, lung cancer, breast cancer, pancreatic cancer, stomach cancer, epithelial ovarian cancer, cholectoral cancer, liver cancer, skin cancer, prostate cancer, cervical cancer, ovarian cancer, bladder cancer, oral cavity cancer, and combinations thereof. In other embodiments, use of the system treats one or more inflammatory diseases, conditions, and illnesses, including, but not limited to, autoinflammatory disease (AID), cryopyrin-associated periodic syndrome (CAPS), systemic lupus erythematosus (SLE), inflammatory bowel disease (IBD) and combinations thereof. In some embodiments, use of the system treats one or more cardiac diseases, conditions, and illnesses, including, but not limited to, myocardial infarction, heart failure (e.g., refractory or acute decompensated heart failure), arrythmias, pericarditis (e.g., refractory idiopathic pericarditis), myocarditis, sepsis-induced cardiomyopathy, atherosclerosis, coronary artery disease and combinations thereof. In some embodiments, use of the system treats one or more neurological diseases, conditions, and illnesses, including, but not limited to, acute thrombotic stroke, epilepsy, multiple sclerosis, Alzheimer's Disease and combinations thereof. In some embodiments, use of the system treats one or more ophthalmic diseases, conditions, and illnesses, including, but not limited to, uveitis, scleritis, Sjogren asthenia, dry eye and combinations thereof. In some embodiments, use of the system treats one or more diseases, conditions, and illnesses, including, but not limited to, chronic kidney disease, Type 2 diabetes, gastroesophageal reflux disease (GERD), non-HP-associated peptic ulcer disease, and pulmonary fibrosis.

### C. Virus-Like Particles

In one aspect, the present disclosure encompasses means for delivering a CRISPR gene-editing system to a mammalian cell via a virus-like particle (VLP). In some embodiments, a CRISPR gene-editing system is delivered by a VLP. Without wishing to be bound by any particular theory, in certain embodiments, nucleic acids, when present in the particle, are resistant in aqueous solution to degradation with a nuclease. In other embodiments, proteins are protected from protease degradation while present in the particle. In some embodiments, proteins and nucleic acids encapsulated by VLPs are capable of penetrating the cellular plasma membrane.

In some embodiments, the CRISPR gene-editing system comprises one or more RNA-containing compositions. In some embodiments, the CRISPR gene-editing system further comprises one or more VLPs. In some embodiments, said one or more RNA-containing compositions comprises a guide RNA. In some embodiments, said one or more RNA-containing compositions comprises an mRNA. In some embodiments, said one or more RNA-containing compositions comprises an RNP (e.g., Cas9 and a guide RNA).

In some embodiments, the CRISPR gene-editing system comprises one or more virus-like particles collectively encapsulating (i) the RNA-guided nuclease or the nucleic acid encoding the RNA-guided nuclease and (ii) the at least one guide RNA or the nucleic acid encoding the at least one guide RNA. In some embodiments, the one or more virus-like particles comprises a first plurality of virus-like particles encapsulating the RNA-guided nuclease or a nucleic acid encoding an RNA-guided nuclease and a second plurality of virus-like particles encapsulating the at least one guide RNA or a nucleic acid encoding at least one guide RNA.

In some embodiments, use of the CRISPR gene-editing system further comprising one or more LNPs to target a gene selected from IL1R1, IL1RAP, TGFBR1, TGFBR2, IL6R, IL6ST, TNFRSF1A, TNFRSF1B, TNFRSF3, TNFRSF4, TNFRSF11A, and combinations thereof is therapeutic.

In one aspect, use of the CRISPR gene-editing system further comprising one or more VLPs to target TGFBR1 and/or TGFBR2 is therapeutic. In some embodiments, use of the system treats one or more joint disease or illness or one or more back or spine conditions or disorders. In some embodiments, use of the system treats one or more musculoskeletal diseases, conditions, and illnesses, including, but not limited to, Loeys-Dietz Syndrome, osteoarthritis, Marfan syndrome, aortic aneurysm (e.g., familial thoracic 3 aortic aneurysm), craniofacial abnormalities, and combinations thereof. In other embodiments, use of the system treats neoplastic diseases, conditions, and illnesses, including, but not limited to, pancreatic cancer, multiple self-healing squamous epithelioma (Ferguson-Smith disease), gastrointestinal stromal tumors (GIST), hereditary nonpolyposis colorectal cancer (Lynch Syndrome), metastatic colorectal carcinoma, bone neoplasms, anaplastic carcinoma, spindle-cell carcinoma, lung neoplasms, brain neoplasms, and combinations thereof.

In some embodiments, the CRISPR gene-editing system further comprising one or more VLPs to target IL1R1 and/or IL1RAP is therapeutic. In some embodiments, use of the system treats one or more joint disease or illness or one or more back or spine conditions or disorders. In some embodiments, use of the system treats one or more musculoskeletal diseases, conditions, and illnesses, including, but not limited to, rheumatoid arthritis, gout, osteoarthritis, osteoporosis, intervertebral disc disease (IVDD), psoriatic arthritis, arthritis, polymyositis, proliferative synovitis, bone neoplasms, sarcoid myopathy, cortex bone disorders, idiopathic scoliosis, tendinopathy, myofibrillar myopathy, enthesis-related arthritis, ankylosing spondylitis, degenerative polyarthritis, arthropathy, osteitis deformans, prolapsed lumbar disc, polymyositis ossificans, idiopathic polymyositis, Luft Disease, adult-onset Still's Disease, osteoarthritis deformans, Bachet's Disease, and combinations thereof. In other embodiments, use of the system treats one or more neoplastic diseases, conditions, and illnesses, including, but not limited to, osteosarcoma, multiple myeloma, lymphoma (e.g., B-cell or cutaneous T-cell lymphoma), leukemia, thyroid carcinoma, glioma, renal cell carcinoma, chondrosarcoma, glioblastoma, melanoma, neuroblastoma, polycystic ovary syndrome, Kaposi sarcoma, squamous cell carcinoma, leiomyosarcoma, malignant peripheral nerve sheath tumor, Ewing's sarcoma, esophageal neoplasms, colon cancer, lung cancer, breast cancer, pancreatic cancer, stomach cancer, epithelial ovarian cancer, cholectoral cancer, liver cancer, skin cancer, prostate cancer, cervical cancer, ovarian cancer, bladder cancer, oral cavity cancer, and combinations thereof. In other embodiments, use of the system treats one or more inflammatory diseases, conditions, and illnesses, including, but not limited to, autoinflammatory disease (AID), cryopyrin-associated periodic syndrome (CAPS), systemic lupus erythematosus (SLE), inflammatory bowel disease (IBD) and combinations thereof. In some embodiments, use of the system treats one or more cardiac diseases, conditions, and illnesses, including, but not limited to, myocardial infarction, heart failure (e.g., refractory or acute decompensated heart failure), arrythmias, pericarditis (e.g., refractory idiopathic pericarditis), myocarditis, sepsis-induced cardiomyopathy, atherosclerosis, coronary artery disease and combinations thereof. In some embodiments, use of the system treats one or more neurological diseases, conditions, and illnesses, including, but not limited to, acute thrombotic stroke, epilepsy, multiple sclerosis, Alzheimer's Disease and combinations thereof. In some embodiments, use of the system treats one or more ophthalmic diseases, conditions, and illnesses, including, but not limited to, uveitis, scleritis, Sjogren asthenia, dry eye and combinations thereof. In some embodiments, use of the system treats one or more diseases, conditions, and illnesses, including, but not limited to, chronic kidney disease, Type 2 diabetes, gastroesophageal reflux disease (GERD), non-HP-associated peptic ulcer disease, and pulmonary fibrosis.

### D. Miscellaneous Modes of Delivery

### 1. Liposomes

In some embodiments, nucleic acids encoding a CRISPR gene-editing system targeting a gene selected from IL1R1, IL1R2, IL1RAP, IL1RL1, TGFBR1, TGFBR2, IL6R, IL6ST, TNFRSF1A, TNFRSF1B, TNFRSF3, TNFRSF4, TNFRSF11A, and combinations thereof (e.g., Cas9 or gRNA) are entrapped in liposomes bearing positive charges on their surface (e.g., lipofectins), which can be tagged with antibodies against cell surface antigens of the target cells. These delivery vehicles can also be used to deliver Cas9 protein/gRNA complexes.

In some embodiments, the CRISPR gene-editing system comprises one or more RNA-containing compositions. In some embodiments, the CRISPR gene-editing system further comprises one or more liposomes. In some embodiments, said one or more RNA-containing compositions comprises a guide RNA. In some embodiments, said one or more RNA-containing compositions comprises an mRNA. In some embodiments, said one or more RNA-containing compositions comprises an RNP (e.g., Cas9 and a guide RNA).

In some embodiments, wherein the composition comprises one or more liposomes collectively encapsulating (i) the RNA-guided nuclease or the nucleic acid encoding the RNA-guided nuclease and (ii) the at least one guide RNA or the nucleic acid encoding the at least one guide RNA. In some embodiments, the one or more liposomes comprises a first plurality of liposomes encapsulating the RNA-guided nuclease or a nucleic acid encoding an RNA-guided nuclease and a second plurality of liposomes encapsulating the at least one guide RNA or a nucleic acid encoding at least one guide RNA.

In some embodiments, use of the CRISPR gene-editing system further comprising one or more LNPs to target a gene selected from IL1R1, IL1RAP, TGFBR1, TGFBR2, IL6R, IL6ST, TNFRSF1A, TNFRSF1B, TNFRSF3, TNFRSF4, TNFRSF11A, and combinations thereof is therapeutic.

In one aspect, use of the CRISPR gene-editing system further comprising one or more liposomes to target TGFBR1 and/or TGFBR2 is therapeutic. In some embodiments, use of the system treats one or more joint disease or illness or one or more back or spine conditions or disorders. In some embodiments, use of the system treats one or more musculoskeletal diseases, conditions, and illnesses, including, but not limited to, Loeys-Dietz Syndrome, osteoarthritis, Marfan syndrome, aortic aneurysm (e.g., familial thoracic 3 aortic aneurysm), craniofacial abnormalities, and combinations thereof. In other embodiments, use of the system treats neoplastic diseases, conditions, and illnesses, including, but not limited to, pancreatic cancer, multiple self-healing squamous epithelioma (Ferguson-Smith disease), gastrointestinal stromal tumors (GIST), hereditary nonpolyposis colorectal cancer (Lynch Syndrome), metastatic colorectal carcinoma, bone neoplasms, anaplastic carcinoma, spindle-cell carcinoma, lung neoplasms, brain neoplasms, and combinations thereof.

In some embodiments, the CRISPR gene-editing system further comprising one or more liposomes to target IL1R1 and/or IL1RAP is therapeutic. In some embodiments, use of the system treats one or more joint disease or illness or one or more back or spine conditions or disorders. In some embodiments, use of the system treats one or more musculoskeletal diseases, conditions, and illnesses, including, but not limited to, rheumatoid arthritis, gout, osteoarthritis, osteoporosis, intervertebral disc disease (IVDD), psoriatic arthritis, arthritis, polymyositis, proliferative synovitis, bone neoplasms, sarcoid myopathy, cortex bone disorders, idiopathic scoliosis, tendinopathy, myofibrillar myopathy, enthesis-related arthritis, ankylosing spondylitis, degenerative polyarthritis, arthropathy, osteitis deformans, prolapsed lumbar disc, polymyositis ossificans, idiopathic polymyositis, Luft Disease, adult-onset Still's Disease, osteoarthritis deformans, Bachet's Disease, and combinations thereof. In other embodiments, use of the system treats one or more neoplastic diseases, conditions, and illnesses, including, but not limited to, osteosarcoma, multiple myeloma, lymphoma (e.g., B-cell or cutaneous T-cell lymphoma), leukemia, thyroid carcinoma, glioma, renal cell carcinoma, chondrosarcoma, glioblastoma, melanoma, neuroblastoma, polycystic ovary syndrome, Kaposi sarcoma, squamous cell carcinoma, leiomyosarcoma, malignant peripheral nerve sheath tumor, Ewing's sarcoma, esophageal neoplasms, colon cancer, lung cancer, breast cancer, pancreatic cancer, stomach cancer, epithelial ovarian cancer, cholectoral cancer, liver cancer, skin cancer, prostate cancer, cervical cancer, ovarian cancer, bladder cancer, oral cavity cancer, and combinations thereof. In other embodiments, use of the system treats one or more inflammatory diseases, conditions, and illnesses, including, but not limited to, autoinflammatory disease (AID), cryopyrin-associated periodic syndrome (CAPS), systemic lupus erythematosus (SLE), inflammatory bowel disease (IBD) and combinations thereof. In some embodiments, use of the system treats one or more cardiac diseases, conditions, and illnesses, including, but not limited to, myocardial infarction, heart failure (e.g., refractory or acute decompensated heart failure), arrythmias, pericarditis (e.g., refractory idiopathic pericarditis), myocarditis, sepsis-induced cardiomyopathy, atherosclerosis, coronary artery disease and combinations thereof. In some embodiments, use of the system treats one or more neurological diseases, conditions, and illnesses, including, but not limited to, acute thrombotic stroke, epilepsy, multiple sclerosis, Alzheimer's Disease and combinations thereof. In some embodiments, use of the system treats one or more ophthalmic diseases, conditions, and illnesses, including, but not limited to, uveitis, scleritis, Sjogren asthenia, dry eye and combinations thereof. In some embodiments, use of the system treats one or more diseases, conditions, and illnesses, including, but not limited to, chronic kidney disease, Type 2 diabetes, gastroesophageal reflux disease (GERD), non-HP-associated peptic ulcer disease, and pulmonary fibrosis.

### 2. Lipid Nanocrystals (LNC)

In one aspect, the In one aspect, the present disclosure encompasses means for delivering a CRISPR gene-editing system to a mammalian cell via a lipid nanocrystal (LNC). In some embodiments, a CRISPR gene-editing system is delivered by a LNC. Without wishing to be bound by any particular theory, in certain embodiments, nucleic acids, when present in the nanocrystal, are resistant in aqueous solution to degradation with a nuclease. In other embodiments, proteins are protected from protease degradation while present in the nanocrystal. In some embodiments, proteins and nucleic acids encapsulated by nanocrystal are capable of penetrating the cellular plasma membrane.

In some embodiments, the CRISPR gene-editing system comprises one or more RNA-containing compositions. In some embodiments, the CRISPR gene-editing system further comprises one or more nanocrystals. In some embodiments, said one or more RNA-containing compositions comprises a guide RNA. In some embodiments, said one or more RNA-containing compositions comprises an mRNA. In some embodiments, said one or more RNA-containing compositions comprises an RNP (e.g., Cas9 and a guide RNA). In some embodiments, said one or more nanocrystals are lipid nanocrystals (LNC).

In some embodiments, the CRISPR gene-editing system comprises one or more LNCs collectively encapsulating (i) the RNA-guided nuclease or the nucleic acid encoding the RNA-guided nuclease and (ii) the at least one guide RNA or the nucleic acid encoding the at least one guide RNA. In some embodiments, the one or more LNCs comprises a first plurality of LNC encapsulating the RNA-guided nuclease or a nucleic acid encoding an RNA-guided nuclease and a second plurality of LNC encapsulating the at least one guide RNA or a nucleic acid encoding at least one guide RNA.

In some embodiments, use of the CRISPR gene-editing system further comprising one or more LNPs to target a gene selected from IL1R1, IL1RAP, TGFBR1, TGFBR2, IL6R, IL6ST, TNFRSF1A, TNFRSF1B, TNFRSF3, TNFRSF4, TNFRSF11A, and combinations thereof is therapeutic.

In one aspect, use of the CRISPR gene-editing system further comprising one or more LNCs to target TGFBR1 and/or TGFBR2 is therapeutic. In some embodiments, use of the system treats one or more joint disease or illness or one or more back or spine conditions or disorders. In some embodiments, use of the system treats one or more musculoskeletal diseases, conditions, and illnesses, including, but not limited to, Loeys-Dietz Syndrome, osteoarthritis, Marfan syndrome, aortic aneurysm (e.g., familial thoracic 3 aortic aneurysm), craniofacial abnormalities, and combinations thereof. In other embodiments, use of the system treats neoplastic diseases, conditions, and illnesses, including, but not limited to, pancreatic cancer, multiple self-healing squamous epithelioma (Ferguson-Smith disease), gastrointestinal stromal tumors (GIST), hereditary nonpolyposis colorectal cancer (Lynch Syndrome), metastatic colorectal carcinoma, bone neoplasms, anaplastic carcinoma, spindle-cell carcinoma, lung neoplasms, brain neoplasms, and combinations thereof.

In some embodiments, the CRISPR gene-editing system further comprising one or more LNCs to target IL1R1 and/or IL1RAP is therapeutic. In some embodiments, use of the system treats one or more joint disease or illness or one or more back or spine conditions or disorders. In some embodiments, use of the system treats one or more musculoskeletal diseases, conditions, and illnesses, including, but not limited to, rheumatoid arthritis, gout, osteoarthritis, osteoporosis, intervertebral disc disease (IVDD), psoriatic arthritis, arthritis, polymyositis, proliferative synovitis, bone neoplasms, sarcoid myopathy, cortex bone disorders, idiopathic scoliosis, tendinopathy, myofibrillar myopathy, enthesis-related arthritis, ankylosing spondylitis, degenerative polyarthritis, arthropathy, osteitis deformans, prolapsed lumbar disc, polymyositis ossificans, idiopathic polymyositis, Luft Disease, adult-onset Still's Disease, osteoarthritis deformans, Bachet's Disease, and combinations thereof. In other embodiments, use of the system treats one or more neoplastic diseases, conditions, and illnesses, including, but not limited to, osteosarcoma, multiple myeloma, lymphoma (e.g., B-cell or cutaneous T-cell lymphoma), leukemia, thyroid carcinoma, glioma, renal cell carcinoma, chondrosarcoma, glioblastoma, melanoma, neuroblastoma, polycystic ovary syndrome, Kaposi sarcoma, squamous cell carcinoma, leiomyosarcoma, malignant peripheral nerve sheath tumor, Ewing's sarcoma, esophageal neoplasms, colon cancer, lung cancer, breast cancer, pancreatic cancer, stomach cancer, epithelial ovarian cancer, cholectoral cancer, liver cancer, skin cancer, prostate cancer, cervical cancer, ovarian cancer, bladder cancer, oral cavity cancer, and combinations thereof. In other embodiments, use of the system treats one or more inflammatory diseases, conditions, and illnesses, including, but not limited to, autoinflammatory disease (AID), cryopyrin-associated periodic syndrome (CAPS), systemic lupus erythematosus (SLE), inflammatory bowel disease (IBD) and combinations thereof. In some embodiments, use of the system treats one or more cardiac diseases, conditions, and illnesses, including, but not limited to, myocardial infarction, heart failure (e.g., refractory or acute decompensated heart failure), arrythmias, pericarditis (e.g., refractory idiopathic pericarditis), myocarditis, sepsis-induced cardiomyopathy, atherosclerosis, coronary artery disease and combinations thereof. In some embodiments, use of the system treats one or more neurological diseases, conditions, and illnesses, including, but not limited to, acute thrombotic stroke, epilepsy, multiple sclerosis, Alzheimer's Disease and combinations thereof. In some embodiments, use of the system treats one or more ophthalmic diseases, conditions, and illnesses, including, but not limited to, uveitis, scleritis, Sjogren asthenia, dry eye and combinations thereof. In some embodiments, use of the system treats one or more diseases, conditions, and illnesses, including, but not limited to, chronic kidney disease, Type 2 diabetes, gastroesophageal reflux disease (GERD), non-HP-associated peptic ulcer disease, and pulmonary fibrosis.

### VII. Pharmaceutical Compositions

In one aspect, the present disclosure encompasses pharmaceutical compositions comprising a CRISPR gene-editing system for treatment of a mammal in need thereof. In some embodiments, the CRISPR gene-editing system targets a gene selected from IL1R1, IL1R2, IL1RAP, IL1RL1, TGFBR1, TGFBR2, IL6R, IL6ST, TNFRSF1A, TNFRSF1B, TNFRSF3, TNFRSF4, TNFRSF11A, and combinations thereof. In some embodiments, the mammal is selected from a human, a dog, a horse, and a cat.

### A. IL1R1

In some embodiments, the pharmaceutical composition comprising a CRISPR gene-editing system targets IL1R1. In some embodiments, the CRISPR gene-editing system targeting IL1R1 is delivered to a mammalian cell via viral vector. In some embodiments, the CRISPR gene-editing system targeting IL1R1 is delivered to a mammalian cell via an AAV vector. In some embodiments, the CRISPR gene-editing system targeting IL1R1 is delivered to a mammalian cell via a lentiviral vector. In some embodiments, the CRISPR gene-editing system targeting IL1R1 is delivered to a mammalian cell via a lipid nanoparticle. In some embodiments, the CRISPR gene-editing system targeting IL1R1 is delivered to a mammalian cell via a virus-like particle. In some embodiments, the CRISPR gene-editing system targeting IL1R1 is delivered to a mammalian cell via a liposome. In some embodiments, the CRISPR gene-editing system targeting IL1R1 is delivered to a mammalian cell via a lipid nanocrystal.

In various embodiments, the pharmaceutical composition comprising a CRISPR gene-editing system that targets the IL1R1 gene is used in a method of treating a mammal in need thereof. In some embodiments, the method treats one or more joint disease or illness or one or more back or spine conditions or disorders. In some embodiments, the method treats one or more musculoskeletal diseases, conditions, and illnesses, including, but not limited to, rheumatoid arthritis, gout, osteoarthritis, osteoporosis, intervertebral disc disease (IVDD), psoriatic arthritis, arthritis, polymyositis, proliferative synovitis, bone neoplasms, sarcoid myopathy, cortex bone disorders, idiopathic scoliosis, tendinopathy, myofibrillar myopathy, enthesis-related arthritis, ankylosing spondylitis, degenerative polyarthritis, arthropathy, osteitis deformans, prolapsed lumbar disc, polymyositis ossificans, idiopathic polymyositis, Luft Disease, adult-onset Still's Disease, osteoarthritis deformans, Bachet's Disease, and combinations thereof. In other embodiments, use of the system treats one or more neoplastic diseases, conditions, and illnesses, including, but not limited to, osteosarcoma, multiple myeloma, lymphoma (e.g., B-cell or cutaneous T-cell lymphoma), leukemia, thyroid carcinoma, glioma, renal cell carcinoma, chondrosarcoma, glioblastoma, melanoma, neuroblastoma, polycystic ovary syndrome, Kaposi sarcoma, squamous cell carcinoma, leiomyosarcoma, malignant peripheral nerve sheath tumor, Ewing's sarcoma, esophageal neoplasms, colon cancer, lung cancer, breast cancer, pancreatic cancer, stomach cancer, epithelial ovarian cancer, cholectoral cancer, liver cancer, skin cancer, prostate cancer, cervical cancer, ovarian cancer, bladder cancer, oral cavity cancer, and combinations thereof. In other embodiments, the method treats one or more inflammatory diseases, conditions, and illnesses, including, but not limited to, autoinflammatory disease (AID), cryopyrin-associated periodic syndrome (CAPS), systemic lupus erythematosus (SLE), inflammatory bowel disease (IBD) and combinations thereof. In some embodiments, the method treats one or more cardiac diseases, conditions, and illnesses, including, but not limited to, myocardial infarction, heart failure (e.g., refractory or acute decompensated heart failure), arrythmias, pericarditis (e.g., refractory idiopathic pericarditis), myocarditis, sepsis-induced cardiomyopathy, atherosclerosis, coronary artery disease and combinations thereof. In some embodiments, the method treats one or more neurological diseases, conditions, and illnesses, including, but not limited to, acute thrombotic stroke, epilepsy, multiple sclerosis, Alzheimer's Disease and combinations thereof. In some embodiments, the method treats one or more ophthalmic diseases, conditions, and illnesses, including, but not limited to, uveitis, scleritis, Sjogren asthenia, dry eye and combinations thereof. In some embodiments, the method treats one or more diseases, conditions, and illnesses, including, but not limited to, chronic kidney disease, Type 2 diabetes, gastroesophageal reflux disease (GERD), non-HP-associated peptic ulcer disease, and pulmonary fibrosis.

In some embodiments, the pharmaceutical composition comprising a CRISPR gene-editing system targets IL1R1 for the treatment of a joint disease or illness. In some embodiments, the pharmaceutical composition comprising a CRISPR gene-editing system targets IL1R1 for the treatment of osteoarthritis. In some embodiments, the pharmaceutical composition comprising a CRISPR gene-editing system targets IL1R1 for the treatment of gout.

In some embodiments, the pharmaceutical composition comprising a CRISPR gene-editing system targets IL1R1 for the treatment of a musculoskeletal disorder.

In some embodiments, the pharmaceutical composition comprising a CRISPR gene-editing system targets IL1R1 for the treatment of a back or spine condition or disorder. In some embodiments, the pharmaceutical composition comprising a CRISPR gene-editing system targets IL1R1 for the treatment of low back pain. In some embodiments, the pharmaceutical composition comprising a CRISPR gene-editing system targets IL1R1 for the treatment of degenerative disc disorder. In some embodiments, the pharmaceutical composition comprising a CRISPR gene-editing system targets IL1R1 for the treatment of internal disc disruption.

### 1. Treatment of Musculoskeletal Disorders by Targeting an Interleukin-1 Receptor Gene

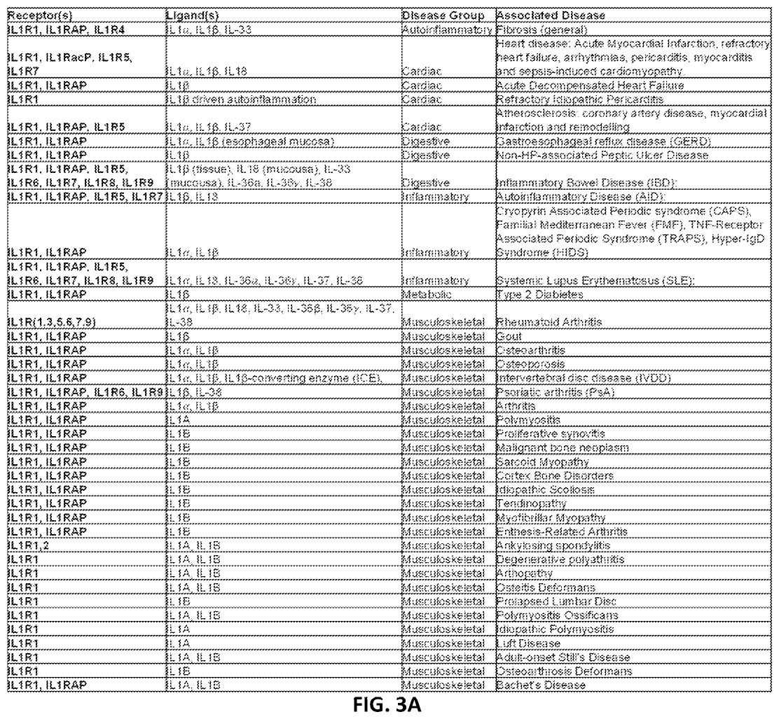

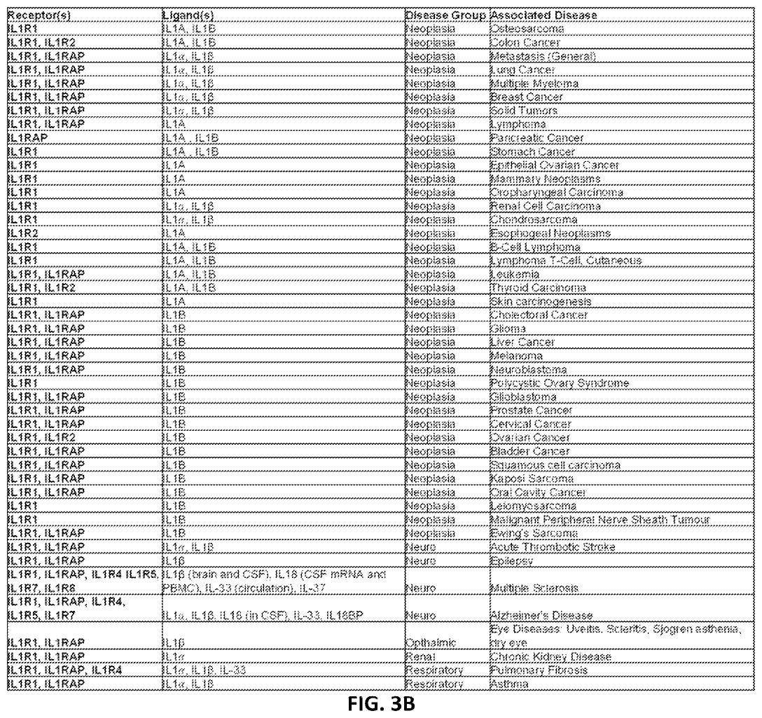

In one aspect, methods and pharmaceutical compositions are provided for treating a musculoskeletal disorder using a CRISPR pharmaceutical composition that targets an interleukin-1 receptor gene. In some embodiments, the CRISPR pharmaceutical composition comprises (i) an RNA-guided nuclease or a nucleic acid encoding an RNA-guided nuclease, and (ii) at least one guide RNA (gRNA), or a nucleic acid encoding at least one gRNA, targeting the interleukin-1 receptor gene, where the gRNA specifically binds a target sequence that is adjacent to a protospacer adjacent motif (PAM) sequence for the RNA-guided nuclease. In some embodiments, the CRISPR pharmaceutical composition comprises (i) an mRNA encoding an RNA-guided nuclease, and (ii) at least one gRNA targeting the interleukin-1 receptor gene. In some embodiments, the mRNA encoding the RNA-guided nuclease and the at least one gRNA are packaged in a lipid nanoparticle (LNP). In some embodiments, the musculoskeletal disorder is Rheumatoid Arthritis, Gout, Osteoarthritis, Osteoporosis, Intervertebral disc disease (IVDD), Psoriatic arthritis (PSA), Arthritis, Polymyositis, Proliferative synovitis, Malignant bone neoplasm, Sarcoid Myopathy, Cortex Bone Disorders, Idiopathic Scoliosis, Tendinopathy, Myofibrillar Myopathy, Enthesis-Related Arthritis, Ankylosing spondylitis, Degenerative, polyarthritis, Arthropathy, Osteitis Deformans, Prolapsed Lumbar Disc, Polymyositis Ossificans, Idiopathic Polymyositis, Luft Disease, Adult-onset Still's Disease, Osteoarthrosis Deformans, or Bachet's Disease. In some embodiments, a musculoskeletal disorder listed in FIG. 3 is treated with a CRISPR pharmaceutical composition targeting a gene listed as associated with the disorder in FIG. 3. For example, in some embodiments, Rheumatoid Arthritis is treated with a CRISPR pharmaceutical composition targeting an IL1R1, IL1RAP, IL1R5, IL1R6, IL1R7, or IL1R9 gene.

In some embodiments, the interleukin-1 receptor gene is an IL1R1 gene. In some embodiments, the IL1R1 gene is a human IL1R1 gene and the guide RNA comprises a crRNA sequence selected from SEQ ID NOs: 680-824. In some embodiments, the IL1R1 gene is a canine IL1R1 gene and the guide RNA comprises a crRNA sequence selected from SEQ ID NOs: 825-892 and SEQ ID NOs: 3336-3420. In some embodiments, the IL1R1 gene is an equine IL1R1 gene and the guide RNA comprises a crRNA sequence selected from SEQ ID NOs: 893-967. In some embodiments, the IL1R1 gene is a feline IL1R1 gene and the guide RNA comprises a crRNA sequence selected from SEQ ID NOs: 968-1039. In some embodiments, editing of the IL1R1 gene results in a knockout of the IL1R1 gene. In some such embodiments, the IL1R1 gene human IL1R1 gene and is a the guide RNA comprises a crRNA sequence selected from SEQ ID NOs: 680-758. In some such embodiments, the IL1R1 gene is a canine IL1R1 gene and the guide RNA comprises a crRNA sequence selected from SEQ ID NOs: 825-892. In some such embodiments, the IL1R1 gene is an equine IL1R1 gene and the guide RNA comprises a crRNA sequence selected from SEQ ID NOs: 893-922. In some such embodiments, the IL1R1 gene is a feline IL1R1 gene and the guide RNA comprises a crRNA sequence selected from SEQ ID NOs: 968-997. In some embodiments, editing of the IL1R1 gene results in a gene that expresses a membrane-bound receptor decoy protein. In some such embodiments, the IL1R1 gene is a human IL1R1 gene and the guide RNA comprises a crRNA sequence selected from SEQ ID NOs: 759-808. In some such embodiments, the IL1R1 gene is a canine IL1R1 gene and the guide RNA comprises a crRNA sequence selected from SEQ ID NOs: 3336-3401. In some such embodiments, the IL1R1 gene is an equine IL1R1 gene and the guide RNA comprises a crRNA sequence selected from SEQ ID NOs: 942-967. In some such embodiments, the IL1R1 gene is a feline IL1R1 gene and the guide RNA comprises a crRNA sequence selected from SEQ ID NOs: 1010-1039. In some embodiments, editing of the IL1R1 gene results in a gene that expresses a soluble receptor decoy protein. In some such embodiments, the IL1R1 gene is a human IL1R1 gene and the guide RNA comprises a crRNA sequence selected from SEQ ID NOs 758 and 809-811. In some such embodiments, the IL1R1 gene is a canine IL1R1 gene and the guide RNA comprises a crRNA sequence selected from SEQ ID NOs: 865-892. and SEQ ID NOS: 3402-3405. In some such embodiments, the IL1R1 gene is an equine IL1R1 gene and the guide RNA comprises a crRNA sequence selected from SEQ ID NOs: 923-941. In some such embodiments, the IL1R1 gene is a feline IL1R1 gene and the guide RNA comprises a crRNA sequence selected from SEQ ID NOs: 998-1009.

### 2. Treatment of Neoplasia by Targeting an Interleukin-1 Receptor Gene

In In one aspect, methods and pharmaceutical compositions are provided for treating a neoplasia using a CRISPR pharmaceutical composition that targets an interleukin-1 receptor gene. In some embodiments, the CRISPR pharmaceutical composition comprises (i) an RNA-guided nuclease or a nucleic acid encoding an RNA-guided nuclease, and (ii) at least one guide RNA (gRNA), or a nucleic acid encoding at least one gRNA, targeting the interleukin-1 receptor gene, where the gRNA specifically binds a target sequence that is adjacent to a protospacer adjacent motif (PAM) sequence for the RNA-guided nuclease. In some embodiments, the CRISPR pharmaceutical composition comprises (i) an mRNA encoding an RNA-guided nuclease, and (ii) at least one gRNA targeting the interleukin-1 receptor gene. In some embodiments, the mRNA encoding the RNA-guided nuclease and the at least one gRNA are packaged in a lipid nanoparticle (LNP). In some embodiments, the neoplasia is Osteosarcoma, Colon Cancer, Metastasis (General), Lung Cancer, Multiple Myeloma. Breast Cancer, Solid Tumors, Lymphoma, Pancreatic Cancer, Stomach Cancer, Epithelial Ovarian Cancer, Mammary Neoplasms, Oropharyngeal Carcinoma, Renal Cell Carcinoma, Chondrosarcoma, Esophageal Neoplasms, B-Cell Lymphoma, Cutaneous Lymphoma T-Cell, Leukemia, Thyroid Carcinoma, Skin carcinogenesis, Cholectoral Cancer, Glioma, Liver Cancer, Melanoma, Neuroblastoma, Polycystic Ovary Syndrome, Glioblastoma, Prostate Cancer, Cervical Cancer, Ovarian Cancer, Bladder Cancer, Squamous cell carcinoma, Kaposi Sarcoma, Oral Cavity Cancer, Leiomyosarcoma, Malignant Peripheral Nerve Sheath Tumor, or Ewing's Sarcoma. In some embodiments, a neoplasia listed in FIG. 3 is treated with a CRISPR pharmaceutical composition targeting a gene listed as associated with the disorder in FIG. 3. For example, in some embodiments, Osteosarcoma is treated with a CRISPR pharmaceutical composition targeting an IL1R1 gene.

In some embodiments, the interleukin-1 receptor gene is an IL1R1 gene. In some embodiments, the IL1R1 gene is a human IL1R1 gene and the guide RNA comprises a crRNA sequence selected from SEQ ID NOs: 680-824. In some embodiments, the IL1R1 gene is a canine IL1R1 gene and the guide RNA comprises a crRNA sequence selected from SEQ ID NOs: 825-892 and SEQ ID NOs: 3336-3420. In some embodiments, the IL1R1 gene is an equine IL1R1 gene and the guide RNA comprises a crRNA sequence selected from SEQ ID NOs: 893-967. In some embodiments, the IL1R1 gene is a feline IL1R1 gene and the guide RNA comprises a crRNA sequence selected from SEQ ID NOs: 968-1039. In some embodiments, editing of the IL1R1 gene results in a knockout of the IL1R1 gene. In some such embodiments, the IL1R1 gene human IL1R1 gene and is a the guide RNA comprises a crRNA sequence selected from SEQ ID NOs: 680-758. In some such embodiments, the IL1R1 gene is a canine IL1R1 gene and the guide RNA comprises a crRNA sequence selected from SEQ ID NOs: 825-892. In some such embodiments, the IL1R1 gene is an equine IL1R1 gene and the guide RNA comprises a crRNA sequence selected from SEQ ID NOs: 893-922. In some such embodiments, the IL1R1 gene is a feline IL1R1 gene and the guide RNA comprises a crRNA sequence selected from SEQ ID NOs: 968-997. In some embodiments, editing of the IL1R1 gene results in a gene that expresses a membrane-bound receptor decoy protein. In some such embodiments, the IL1R1 gene is a human IL1R1 gene and the guide RNA comprises a crRNA sequence selected from SEQ ID NOs: 759-808. In some such embodiments, the IL1R1 gene is a canine IL1R1 gene and the guide RNA comprises a crRNA sequence selected from SEQ ID NOs: 3336-3401. In some such embodiments, the IL1R1 gene is an equine IL1R1 gene and the guide RNA comprises a crRNA sequence selected from SEQ ID NOs: 942-967. In some such embodiments, the IL1R1 gene is a feline IL1R1 gene and the guide RNA comprises a crRNA sequence selected from SEQ ID NOs: 1010-1039. In some embodiments, editing of the IL1R1 gene results in a gene that expresses a soluble receptor decoy protein. In some such embodiments, the IL1R1 gene is a human IL1R1 gene and the guide RNA comprises a crRNA sequence selected from SEQ ID NOs 758 and 809-811. In some such embodiments, the IL1R1 gene is a canine IL1R1 gene and the guide RNA comprises a crRNA sequence selected from SEQ ID NOs: 865-892. and SEQ ID NOS: 3402-3405. In some such embodiments, the IL1R1 gene is an equine IL1R1 gene and the guide RNA comprises a crRNA sequence selected from SEQ ID NOs: 923-941. In some such embodiments, the IL1R1 gene is a feline IL1R1 gene and the guide RNA comprises a crRNA sequence selected from SEQ ID NOs: 998-1009.

### 3. Treatment of Neurological Disorder by Targeting an Interleukin-1 Receptor Gene

In one aspect, methods and pharmaceutical compositions are provided for treating a neurological disorder using a CRISPR pharmaceutical composition that targets an interleukin-1 receptor gene. In some embodiments, the CRISPR pharmaceutical composition comprises (i) an RNA-guided nuclease or a nucleic acid encoding an RNA-guided nuclease, and (ii) at least one guide RNA (gRNA), or a nucleic acid encoding at least one gRNA, targeting the interleukin-1 receptor gene, where the gRNA specifically binds a target sequence that is adjacent to a protospacer adjacent motif (PAM) sequence for the RNA-guided nuclease. In some embodiments, the CRISPR pharmaceutical composition comprises (i) an mRNA encoding an RNA-guided nuclease, and (ii) at least one gRNA targeting the interleukin-1 receptor gene. In some embodiments, the mRNA encoding the RNA-guided nuclease and the at least one gRNA are packaged in a lipid nanoparticle (LNP). In some embodiments, the neurological disorder is Acute Thrombotic Stroke, Epilepsy, Multiple Sclerosis, or Alzheimer's Disease. In some embodiments, a neurological disorder listed in FIG. 3 is treated with a CRISPR pharmaceutical composition targeting a gene listed as associated with the disorder in FIG. 3. For example, in some embodiments, Acute Thrombocytic Stroke is treated with a CRISPR pharmaceutical composition targeting an IL1R1, or an IL1RAP gene.

In some embodiments, the interleukin-1 receptor gene is an IL1R1 gene. In some embodiments, the IL1R1 gene is a human IL1R1 gene and the guide RNA comprises a crRNA sequence selected from SEQ ID NOs: 680-824. In some embodiments, the IL1R1 gene is a canine IL1R1 gene and the guide RNA comprises a crRNA sequence selected from SEQ ID NOs: 825-892 and SEQ ID NOs: 3336-3420. In some embodiments, the IL1R1 gene is an equine IL1R1 gene and the guide RNA comprises a crRNA sequence selected from SEQ ID NOs: 893-967. In some embodiments, the IL1R1 gene is a feline IL1R1 gene and the guide RNA comprises a crRNA sequence selected from SEQ ID NOs: 968-1039. In some embodiments, editing of the IL1R1 gene results in a knockout of the IL1R1 gene. In some such embodiments, the IL1R1 gene human IL1R1 gene and is a the guide RNA comprises a crRNA sequence selected from SEQ ID NOs: 680-758. In some such embodiments, the IL1R1 gene is a canine IL1R1 gene and the guide RNA comprises a crRNA sequence selected from SEQ ID NOs: 825-892. In some such embodiments, the IL1R1 gene is an equine IL1R1 gene and the guide RNA comprises a crRNA sequence selected from SEQ ID NOs: 893-922. In some such embodiments, the IL1R1 gene is a feline IL1R1 gene and the guide RNA comprises a crRNA sequence selected from SEQ ID NOs: 968-997. In some embodiments, editing of the IL1R1 gene results in a gene that expresses a membrane-bound receptor decoy protein. In some such embodiments, the IL1R1 gene is a human IL1R1 gene and the guide RNA comprises a crRNA sequence selected from SEQ ID NOs: 759-808. In some such embodiments, the IL1R1 gene is a canine IL1R1 gene and the guide RNA comprises a crRNA sequence selected from SEQ ID NOs: 3336-3401. In some such embodiments, the IL1R1 gene is an equine IL1R1 gene and the guide RNA comprises a crRNA sequence selected from SEQ ID NOs: 942-967. In some such embodiments, the IL1R1 gene is a feline IL1R1 gene and the guide RNA comprises a crRNA sequence selected from SEQ ID NOs: 1010-1039. In some embodiments, editing of the IL1R1 gene results in a gene that expresses a soluble receptor decoy protein. In some such embodiments, the IL1R1 gene is a human IL1R1 gene and the guide RNA comprises a crRNA sequence selected from SEQ ID NOs 758 and 809-811. In some such embodiments, the IL1R1 gene is a canine IL1R1 gene and the guide RNA comprises a crRNA sequence selected from SEQ ID NOs: 865-892. and SEQ ID NOS: 3402-3405. In some such embodiments, the IL1R1 gene is an equine IL1R1 gene and the guide RNA comprises a crRNA sequence selected from SEQ ID NOs: 923-941. In some such embodiments, the IL1R1 gene is a feline IL1R1 gene and the guide RNA comprises a crRNA sequence selected from SEQ ID NOs: 998-1009.

### 4. Treatment of Cardiac Disorder by Targeting an Interleukin-1 Receptor Gene

In one aspect, methods and pharmaceutical compositions are provided for treating a cardiac disorder using a CRISPR pharmaceutical composition that targets an interleukin-1 receptor gene. In some embodiments, the CRISPR pharmaceutical composition comprises (i) an RNA-guided nuclease or a nucleic acid encoding an RNA-guided nuclease, and (ii) at least one guide RNA (gRNA), or a nucleic acid encoding at least one gRNA, targeting the interleukin-1 receptor gene, where the gRNA specifically binds a target sequence that is adjacent to a protospacer adjacent motif (PAM) sequence for the RNA-guided nuclease. In some embodiments, the CRISPR pharmaceutical composition comprises (i) an mRNA encoding an RNA-guided nuclease, and (ii) at least one gRNA targeting the interleukin-1 receptor gene. In some embodiments, the mRNA encoding the RNA-guided nuclease and the at least one gRNA are packaged in a lipid nanoparticle (LNP). In some embodiments, the cardiac disorder is Heart disease including but not limited to Acute Myocardial Infarction, refractory heart failure, arrhythmias, pericarditis, myocarditis, and sepsis-induced cardiomyopathy; Acute Decompensated Heart Failure, Refractory Idiopathic Pericarditis, or Atherosclerosis including but not limited to coronary artery disease, myocardial infarction, and cardiac remodeling. In some embodiments, a cardiac disorder listed in FIG. 3 is treated with a CRISPR pharmaceutical composition targeting a gene listed as associated with the disorder in FIG. 3. For example, in some embodiments, Acute Decompensated Heart Failure is treated with a CRISPR pharmaceutical composition targeting an IL1R1, or an IL1RAP gene.

In some embodiments, the interleukin-1 receptor gene is an IL1R1 gene. In some embodiments, the IL1R1 gene is a human IL1R1 gene and the guide RNA comprises a crRNA sequence selected from SEQ ID NOs: 680-824. In some embodiments, the IL1R1 gene is a canine IL1R1 gene and the guide RNA comprises a crRNA sequence selected from SEQ ID NOs: 825-892 and SEQ ID NOs: 3336-3420. In some embodiments, the IL1R1 gene is an equine IL1R1 gene and the guide RNA comprises a crRNA sequence selected from SEQ ID NOs: 893-967. In some embodiments, the IL1R1 gene is a feline IL1R1 gene and the guide RNA comprises a crRNA sequence selected from SEQ ID NOs: 968-1039. In some embodiments, editing of the IL1R1 gene results in a knockout of the IL1R1 gene. In some such embodiments, the IL1R1 gene human IL1R1 gene and is a the guide RNA comprises a crRNA sequence selected from SEQ ID NOs: 680-758. In some such embodiments, the IL1R1 gene is a canine IL1R1 gene and the guide RNA comprises a crRNA sequence selected from SEQ ID NOs: 825-892. In some such embodiments, the IL1R1 gene is an equine IL1R1 gene and the guide RNA comprises a crRNA sequence selected from SEQ ID NOs: 893-922. In some such embodiments, the IL1R1 gene is a feline IL1R1 gene and the guide RNA comprises a crRNA sequence selected from SEQ ID NOs: 968-997. In some embodiments, editing of the IL1R1 gene results in a gene that expresses a membrane-bound receptor decoy protein. In some such embodiments, the IL1R1 gene is a human IL1R1 gene and the guide RNA comprises a crRNA sequence selected from SEQ ID NOs: 759-808. In some such embodiments, the IL1R1 gene is a canine IL1R1 gene and the guide RNA comprises a crRNA sequence selected from SEQ ID NOs: 3336-3401. In some such embodiments, the IL1R1 gene is an equine IL1R1 gene and the guide RNA comprises a crRNA sequence selected from SEQ ID NOs: 942-967. In some such embodiments, the IL1R1 gene is a feline IL1R1 gene and the guide RNA comprises a crRNA sequence selected from SEQ ID NOs: 1010-1039. In some embodiments, editing of the IL1R1 gene results in a gene that expresses a soluble receptor decoy protein. In some such embodiments, the IL1R1 gene is a human IL1R1 gene and the guide RNA comprises a crRNA sequence selected from SEQ ID NOs 758 and 809-811. In some such embodiments, the IL1R1 gene is a canine IL1R1 gene and the guide RNA comprises a crRNA sequence selected from SEQ ID NOs: 865-892. and SEQ ID NOS: 3402-3405. In some such embodiments, the IL1R1 gene is an equine IL1R1 gene and the guide RNA comprises a crRNA sequence selected from SEQ ID NOs: 923-941. In some such embodiments, the IL1R1 gene is a feline IL1R1 gene and the guide RNA comprises a crRNA sequence selected from SEQ ID NOs: 998-1009.

### 5. Treatment of Inflammatory Disorders by Targeting an Interleukin-1 Receptor Gene

In one aspect, methods and pharmaceutical compositions are provided for treating an inflammatory disorder using a CRISPR pharmaceutical composition that targets an interleukin-1 receptor gene. In some embodiments, the CRISPR pharmaceutical composition comprises (i) an RNA-guided nuclease or a nucleic acid encoding an RNA-guided nuclease, and (ii) at least one guide RNA (gRNA), or a nucleic acid encoding at least one gRNA, targeting the interleukin-1 receptor gene, where the gRNA specifically binds a target sequence that is adjacent to a protospacer adjacent motif (PAM) sequence for the RNA-guided nuclease. In some embodiments, the CRISPR pharmaceutical composition comprises (i) an mRNA encoding an RNA-guided nuclease, and (ii) at least one gRNA targeting the interleukin-1 receptor gene. In some embodiments, the mRNA encoding the RNA-guided nuclease and the at least one gRNA are packaged in a lipid nanoparticle (LNP). In some embodiments, the inflammatory disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general). In some embodiments, an inflammatory disorder listed in FIG. 3 is treated with a CRISPR pharmaceutical composition targeting a gene listed as associated with the disorder in FIG. 3. For example, in some embodiments, an autoinflammatory disease (AID) is treated with a CRISPR pharmaceutical composition targeting an IL1R1, IL1RAP, IL1R5, or an IL1R7 gene.

In some embodiments, the interleukin-1 receptor gene is an IL1R1 gene. In some embodiments, the IL1R1 gene is a human IL1R1 gene and the guide RNA comprises a crRNA sequence selected from SEQ ID NOs: 680-824. In some embodiments, the IL1R1 gene is a canine IL1R1 gene and the guide RNA comprises a crRNA sequence selected from SEQ ID NOs: 825-892 and SEQ ID NOs: 3336-3420. In some embodiments, the IL1R1 gene is an equine IL1R1 gene and the guide RNA comprises a crRNA sequence selected from SEQ ID NOs: 893-967. In some embodiments, the IL1R1 gene is a feline IL1R1 gene and the guide RNA comprises a crRNA sequence selected from SEQ ID NOs: 968-1039. In some embodiments, editing of the IL1R1 gene results in a knockout of the IL1R1 gene. In some such embodiments, the IL1R1 gene human IL1R1 gene and is a the guide RNA comprises a crRNA sequence selected from SEQ ID NOs: 680-758. In some such embodiments, the IL1R1 gene is a canine IL1R1 gene and the guide RNA comprises a crRNA sequence selected from SEQ ID NOs: 825-892. In some such embodiments, the IL1R1 gene is an equine IL1R1 gene and the guide RNA comprises a crRNA sequence selected from SEQ ID NOs: 893-922. In some such embodiments, the IL1R1 gene is a feline IL1R1 gene and the guide RNA comprises a crRNA sequence selected from SEQ ID NOs: 968-997. In some embodiments, editing of the IL1R1 gene results in a gene that expresses a membrane-bound receptor decoy protein. In some such embodiments, the IL1R1 gene is a human IL1R1 gene and the guide RNA comprises a crRNA sequence selected from SEQ ID NOs: 759-808. In some such embodiments, the IL1R1 gene is a canine IL1R1 gene and the guide RNA comprises a crRNA sequence selected from SEQ ID NOs: 3336-3401. In some such embodiments, the IL1R1 gene is an equine IL1R1 gene and the guide RNA comprises a crRNA sequence selected from SEQ ID NOs: 942-967. In some such embodiments, the IL1R1 gene is a feline IL1R1 gene and the guide RNA comprises a crRNA sequence selected from SEQ ID NOs: 1010-1039. In some embodiments, editing of the IL1R1 gene results in a gene that expresses a soluble receptor decoy protein. In some such embodiments, the IL1R1 gene is a human IL1R1 gene and the guide RNA comprises a crRNA sequence selected from SEQ ID NOs 758 and 809-811. In some such embodiments, the IL1R1 gene is a canine IL1R1 gene and the guide RNA comprises a crRNA sequence selected from SEQ ID NOs: 865-892. and SEQ ID NOS: 3402-3405. In some such embodiments, the IL1R1 gene is an equine IL1R1 gene and the guide RNA comprises a crRNA sequence selected from SEQ ID NOs: 923-941. In some such embodiments, the IL1R1 gene is a feline IL1R1 gene and the guide RNA comprises a crRNA sequence selected from SEQ ID NOs: 998-1009.

### 6. Treatment of Digestive Disorders by Targeting an Interleukin-1 Receptor Gene

In one aspect, methods and pharmaceutical compositions are provided for treating a digestive disorder using a CRISPR pharmaceutical composition that targets an interleukin-1 receptor gene. In some embodiments, the CRISPR pharmaceutical composition comprises (i) an RNA-guided nuclease or a nucleic acid encoding an RNA-guided nuclease, and (ii) at least one guide RNA (gRNA), or a nucleic acid encoding at least one gRNA, targeting the interleukin-1 receptor gene, where the gRNA specifically binds a target sequence that is adjacent to a protospacer adjacent motif (PAM) sequence for the RNA-guided nuclease. In some embodiments, the CRISPR pharmaceutical composition comprises (i) an mRNA encoding an RNA-guided nuclease, and (ii) at least one gRNA targeting the interleukin-1 receptor gene. In some embodiments, the mRNA encoding the RNA-guided nuclease and the at least one gRNA are packaged in a lipid nanoparticle (LNP). In some embodiments, the digestive disorder is Inflammatory Bowel Disease (IBD), Gastroesophageal reflux disease (GERD), or Non-HP-associated Peptic Ulcer Disease. In some embodiments, a digestive disorder listed in FIG. 3 is treated with a CRISPR pharmaceutical composition targeting a gene listed as associated with the disorder in FIG. 3. For example, in some embodiments, an Inflammatory Bowel Disease (IBD) is treated with a CRISPR pharmaceutical composition targeting an IL1R1, IL1RAP, IL1R5, IL1R6, IL1R7, IL1R8, or an IL1R9 gene.

In some embodiments, the interleukin-1 receptor gene is an IL1R1 gene. In some embodiments, the IL1R1 gene is a human IL1R1 gene and the guide RNA comprises a crRNA sequence selected from SEQ ID NOs: 680-824. In some embodiments, the IL1R1 gene is a canine IL1R1 gene and the guide RNA comprises a crRNA sequence selected from SEQ ID NOs: 825-892 and SEQ ID NOs: 3336-3420. In some embodiments, the IL1R1 gene is an equine IL1R1 gene and the guide RNA comprises a crRNA sequence selected from SEQ ID NOs: 893-967. In some embodiments, the IL1R1 gene is a feline IL1R1 gene and the guide RNA comprises a crRNA sequence selected from SEQ ID NOs: 968-1039. In some embodiments, editing of the IL1R1 gene results in a knockout of the IL1R1 gene. In some such embodiments, the IL1R1 gene human IL1R1 gene and is a the guide RNA comprises a crRNA sequence selected from SEQ ID NOs: 680-758. In some such embodiments, the IL1R1 gene is a canine IL1R1 gene and the guide RNA comprises a crRNA sequence selected from SEQ ID NOs: 825-892. In some such embodiments, the IL1R1 gene is an equine IL1R1 gene and the guide RNA comprises a crRNA sequence selected from SEQ ID NOs: 893-922. In some such embodiments, the IL1R1 gene is a feline IL1R1 gene and the guide RNA comprises a crRNA sequence selected from SEQ ID NOs: 968-997. In some embodiments, editing of the IL1R1 gene results in a gene that expresses a membrane-bound receptor decoy protein. In some such embodiments, the IL1R1 gene is a human IL1R1 gene and the guide RNA comprises a crRNA sequence selected from SEQ ID NOs: 759-808. In some such embodiments, the IL1R1 gene is a canine IL1R1 gene and the guide RNA comprises a crRNA sequence selected from SEQ ID NOs: 3336-3401. In some such embodiments, the IL1R1 gene is an equine IL1R1 gene and the guide RNA comprises a crRNA sequence selected from SEQ ID NOs: 942-967. In some such embodiments, the IL1R1 gene is a feline IL1R1 gene and the guide RNA comprises a crRNA sequence selected from SEQ ID NOs: 1010-1039. In some embodiments, editing of the IL1R1 gene results in a gene that expresses a soluble receptor decoy protein. In some such embodiments, the IL1R1 gene is a human IL1R1 gene and the guide RNA comprises a crRNA sequence selected from SEQ ID NOs 758 and 809-811. In some such embodiments, the IL1R1 gene is a canine IL1R1 gene and the guide RNA comprises a crRNA sequence selected from SEQ ID NOs: 865-892. and SEQ ID NOS: 3402-3405. In some such embodiments, the IL1R1 gene is an equine IL1R1 gene and the guide RNA comprises a crRNA sequence selected from SEQ ID NOs: 923-941. In some such embodiments, the IL1R1 gene is a feline IL1R1 gene and the guide RNA comprises a crRNA sequence selected from SEQ ID NOs: 998-1009.

### 7. Treatment of Respiratory Disorders by Targeting an Interleukin-1 Receptor Gene

In one aspect, methods and pharmaceutical compositions are provided for treating a respiratory disorder using a CRISPR pharmaceutical composition that targets an interleukin-1 receptor gene. In some embodiments, the CRISPR pharmaceutical composition comprises (i) an RNA-guided nuclease or a nucleic acid encoding an RNA-guided nuclease, and (ii) at least one guide RNA (gRNA), or a nucleic acid encoding at least one gRNA, targeting the interleukin-1 receptor gene, where the gRNA specifically binds a target sequence that is adjacent to a protospacer adjacent motif (PAM) sequence for the RNA-guided nuclease. In some embodiments, the CRISPR pharmaceutical composition comprises (i) an mRNA encoding an RNA-guided nuclease, and (ii) at least one gRNA targeting the interleukin-1 receptor gene. In some embodiments, the mRNA encoding the RNA-guided nuclease and the at least one gRNA are packaged in a lipid nanoparticle (LNP). In some embodiments, the respiratory disorder is Asthma, or Pulmonary Fibrosis. In some embodiments, a respiratory disorder listed in FIG. 3 is treated with a CRISPR pharmaceutical composition targeting a gene listed as associated with the disorder in FIG. 3. For example, in some embodiments, Asthma is treated with a CRISPR pharmaceutical composition targeting an IL1R1, or IL1RAP gene.

In some embodiments, the interleukin-1 receptor gene is an IL1R1 gene. In some embodiments, the IL1R1 gene is a human IL1R1 gene and the guide RNA comprises a crRNA sequence selected from SEQ ID NOs: 680-824. In some embodiments, the IL1R1 gene is a canine IL1R1 gene and the guide RNA comprises a crRNA sequence selected from SEQ ID NOs: 825-892 and SEQ ID NOs: 3336-3420. In some embodiments, the IL1R1 gene is an equine IL1R1 gene and the guide RNA comprises a crRNA sequence selected from SEQ ID NOs: 893-967. In some embodiments, the IL1R1 gene is a feline IL1R1 gene and the guide RNA comprises a crRNA sequence selected from SEQ ID NOs: 968-1039. In some embodiments, editing of the IL1R1 gene results in a knockout of the IL1R1 gene. In some such embodiments, the IL1R1 gene human IL1R1 gene and is a the guide RNA comprises a crRNA sequence selected from SEQ ID NOs: 680-758. In some such embodiments, the IL1R1 gene is a canine IL1R1 gene and the guide RNA comprises a crRNA sequence selected from SEQ ID NOs: 825-892. In some such embodiments, the IL1R1 gene is an equine IL1R1 gene and the guide RNA comprises a crRNA sequence selected from SEQ ID NOs: 893-922. In some such embodiments, the IL1R1 gene is a feline IL1R1 gene and the guide RNA comprises a crRNA sequence selected from SEQ ID NOs: 968-997. In some embodiments, editing of the IL1R1 gene results in a gene that expresses a membrane-bound receptor decoy protein. In some such embodiments, the IL1R1 gene is a human IL1R1 gene and the guide RNA comprises a crRNA sequence selected from SEQ ID NOs: 759-808. In some such embodiments, the IL1R1 gene is a canine IL1R1 gene and the guide RNA comprises a crRNA sequence selected from SEQ ID NOs: 3336-3401. In some such embodiments, the IL1R1 gene is an equine IL1R1 gene and the guide RNA comprises a crRNA sequence selected from SEQ ID NOs: 942-967. In some such embodiments, the IL1R1 gene is a feline IL1R1 gene and the guide RNA comprises a crRNA sequence selected from SEQ ID NOs: 1010-1039. In some embodiments, editing of the IL1R1 gene results in a gene that expresses a soluble receptor decoy protein. In some such embodiments, the IL1R1 gene is a human IL1R1 gene and the guide RNA comprises a crRNA sequence selected from SEQ ID NOs 758 and 809-811. In some such embodiments, the IL1R1 gene is a canine IL1R1 gene and the guide RNA comprises a crRNA sequence selected from SEQ ID NOs: 865-892. and SEQ ID NOS: 3402-3405. In some such embodiments, the IL1R1 gene is an equine IL1R1 gene and the guide RNA comprises a crRNA sequence selected from SEQ ID NOs: 923-941. In some such embodiments, the IL1R1 gene is a feline IL1R1 gene and the guide RNA comprises a crRNA sequence selected from SEQ ID NOs: 998-1009.

### 8. Treatment of Renal, Metabolic, or Ophthalmic Disorders by Targeting an Interleukin-1 Receptor Gene

In one aspect, methods and pharmaceutical compositions are provided for treating a renal, metabolic, or ophthalmic disorder using a CRISPR pharmaceutical composition that targets an interleukin-1 receptor gene. In some embodiments, the CRISPR pharmaceutical composition comprises (i) an RNA-guided nuclease or a nucleic acid encoding an RNA-guided nuclease, and (ii) at least one guide RNA (gRNA), or a nucleic acid encoding at least one gRNA, targeting the interleukin-1 receptor gene, where the gRNA specifically binds a target sequence that is adjacent to a protospacer adjacent motif (PAM) sequence for the RNA-guided nuclease. In some embodiments, the CRISPR pharmaceutical composition comprises (i) an mRNA encoding an RNA-guided nuclease, and (ii) at least one gRNA targeting the interleukin-1 receptor gene. In some embodiments, the mRNA encoding the RNA-guided nuclease and the at least one gRNA are packaged in a lipid nanoparticle (LNP). In some embodiments, the renal, metabolic, or ophthalmic disorder is Chronic Kidney Disease, Type 2 Diabetes, or Eye Diseases including but not limited to Uveitis, Scleritis, Sjogren asthenia, and dry eye. In some embodiments, a renal, metabolic, or ophthalmic disorder listed in FIG. 3 is treated with a CRISPR pharmaceutical composition targeting a gene listed as associated with the disorder in FIG. 3. For example, in some embodiments, Chronic Kidney disease is treated with a CRISPR pharmaceutical composition targeting an IL1R1, or IL1RAP gene.

In some embodiments, the interleukin-1 receptor gene is an IL1R1 gene. In some embodiments, the IL1R1 gene is a human IL1R1 gene and the guide RNA comprises a crRNA sequence selected from SEQ ID NOs: 680-824. In some embodiments, the IL1R1 gene is a canine IL1R1 gene and the guide RNA comprises a crRNA sequence selected from SEQ ID NOs: 825-892 and SEQ ID NOs: 3336-3420. In some embodiments, the IL1R1 gene is an equine IL1R1 gene and the guide RNA comprises a crRNA sequence selected from SEQ ID NOs: 893-967. In some embodiments, the IL1R1 gene is a feline IL1R1 gene and the guide RNA comprises a crRNA sequence selected from SEQ ID NOs: 968-1039. In some embodiments, editing of the IL1R1 gene results in a knockout of the IL1R1 gene. In some such embodiments, the IL1R1 gene human IL1R1 gene and is a the guide RNA comprises a crRNA sequence selected from SEQ ID NOs: 680-758. In some such embodiments, the IL1R1 gene is a canine IL1R1 gene and the guide RNA comprises a crRNA sequence selected from SEQ ID NOs: 825-892. In some such embodiments, the IL1R1 gene is an equine IL1R1 gene and the guide RNA comprises a crRNA sequence selected from SEQ ID NOs: 893-922. In some such embodiments, the IL1R1 gene is a feline IL1R1 gene and the guide RNA comprises a crRNA sequence selected from SEQ ID NOs: 968-997. In some embodiments, editing of the IL1R1 gene results in a gene that expresses a membrane-bound receptor decoy protein. In some such embodiments, the IL1R1 gene is a human IL1R1 gene and the guide RNA comprises a crRNA sequence selected from SEQ ID NOs: 759-808. In some such embodiments, the IL1R1 gene is a canine IL1R1 gene and the guide RNA comprises a crRNA sequence selected from SEQ ID NOs: 3336-3401. In some such embodiments, the IL1R1 gene is an equine IL1R1 gene and the guide RNA comprises a crRNA sequence selected from SEQ ID NOs: 942-967. In some such embodiments, the IL1R1 gene is a feline IL1R1 gene and the guide RNA comprises a crRNA sequence selected from SEQ ID NOs: 1010-1039. In some embodiments, editing of the IL1R1 gene results in a gene that expresses a soluble receptor decoy protein. In some such embodiments, the IL1R1 gene is a human IL1R1 gene and the guide RNA comprises a crRNA sequence selected from SEQ ID NOs 758 and 809-811. In some such embodiments, the IL1R1 gene is a canine IL1R1 gene and the guide RNA comprises a crRNA sequence selected from SEQ ID NOs: 865-892. and SEQ ID NOS: 3402-3405. In some such embodiments, the IL1R1 gene is an equine IL1R1 gene and the guide RNA comprises a crRNA sequence selected from SEQ ID NOs: 923-941. In some such embodiments, the IL1R1 gene is a feline IL1R1 gene and the guide RNA comprises a crRNA sequence selected from SEQ ID NOs: 998-1009.

### B. IL1RAP

In some embodiments, the pharmaceutical composition comprising a CRISPR gene-editing system targets IL1RAP. In some embodiments, the CRISPR gene-editing system targeting IL1RAP is delivered to a mammalian cell via viral vector. In some embodiments, the CRISPR gene-editing system targeting IL1RAP is delivered to a mammalian cell via an AAV vector. In some embodiments, the CRISPR gene-editing system targeting IL1RAP is delivered to a mammalian cell via a lentiviral vector. In some embodiments, the CRISPR gene-editing system targeting IL1RAP is delivered to a mammalian cell via a lipid nanoparticle. In some embodiments, the CRISPR gene-editing system targeting IL1RAP is delivered to a mammalian cell via a virus-like particle. In some embodiments, the CRISPR gene-editing system targeting IL1RAP is delivered to a mammalian cell via a liposome. In some embodiments, the CRISPR gene-editing system targeting IL1RAP is delivered to a mammalian cell via a lipid nanocrystal.

In various embodiments, the pharmaceutical composition comprising a CRISPR gene-editing system that targets the IL1RAP gene is used in a method of treating a mammal in need thereof. In some embodiments, the method treats one or more joint disease or illness or one or more back or spine conditions or disorders. In some embodiments, the method treats one or more musculoskeletal diseases, conditions, and illnesses, including, but not limited to, rheumatoid arthritis, gout, osteoarthritis, osteoporosis, intervertebral disc disease (IVDD), psoriatic arthritis, arthritis, polymyositis, proliferative synovitis, bone neoplasms, sarcoid myopathy, cortex bone disorders, idiopathic scoliosis, tendinopathy, myofibrillar myopathy, enthesis-related arthritis, ankylosing spondylitis, degenerative polyarthritis, arthropathy, osteitis deformans, prolapsed lumbar disc, polymyositis ossificans, idiopathic polymyositis, Luft Disease, adult-onset Still's Disease, osteoarthritis deformans, Bachet's Disease, and combinations thereof. In other embodiments, use of the system treats one or more neoplastic diseases, conditions, and illnesses, including, but not limited to, osteosarcoma, multiple myeloma, lymphoma (e.g., B-cell or cutaneous T-cell lymphoma), leukemia, thyroid carcinoma, glioma, renal cell carcinoma, chondrosarcoma, glioblastoma, melanoma, neuroblastoma, polycystic ovary syndrome, Kaposi sarcoma, squamous cell carcinoma, leiomyosarcoma, malignant peripheral nerve sheath tumor, Ewing's sarcoma, esophageal neoplasms, colon cancer, lung cancer, breast cancer, pancreatic cancer, stomach cancer, epithelial ovarian cancer, cholectoral cancer, liver cancer, skin cancer, prostate cancer, cervical cancer, ovarian cancer, bladder cancer, oral cavity cancer, and combinations thereof. In other embodiments, the method treats one or more inflammatory diseases, conditions, and illnesses, including, but not limited to, autoinflammatory disease (AID), cryopyrin-associated periodic syndrome (CAPS), systemic lupus erythematosus (SLE), inflammatory bowel disease (IBD) and combinations thereof. In some embodiments, the method treats one or more cardiac diseases, conditions, and illnesses, including, but not limited to, myocardial infarction, heart failure (e.g., refractory or acute decompensated heart failure), arrythmias, pericarditis (e.g., refractory idiopathic pericarditis), myocarditis, sepsis-induced cardiomyopathy, atherosclerosis, coronary artery disease and combinations thereof. In some embodiments, the method treats one or more neurological diseases, conditions, and illnesses, including, but not limited to, acute thrombotic stroke, epilepsy, multiple sclerosis, Alzheimer's Disease and combinations thereof. In some embodiments, the method treats one or more ophthalmic diseases, conditions, and illnesses, including, but not limited to, uveitis, scleritis, Sjogren asthenia, dry eye and combinations thereof. In some embodiments, the method treats one or more diseases, conditions, and illnesses, including, but not limited to, chronic kidney disease, Type 2 diabetes, gastroesophageal reflux disease (GERD), non-HP-associated peptic ulcer disease, and pulmonary fibrosis.

In some embodiments, the pharmaceutical composition comprising a CRISPR gene-editing system targets IL1RAP for the treatment of a joint disease or illness. In some embodiments, the pharmaceutical composition comprising a CRISPR gene-editing system targets IL1RAP for the treatment of osteoarthritis. In some embodiments, the pharmaceutical composition comprising a CRISPR gene-editing system targets IL1RAP for the treatment of gout.

In some embodiments, the pharmaceutical composition comprising a CRISPR gene-editing system targets IL1RAP for the treatment of a musculoskeletal disorder.

In some embodiments, the pharmaceutical composition comprising a CRISPR gene-editing system targets IL1RAP for the treatment of a back or spine condition or disorder. In some embodiments, the pharmaceutical composition comprising a CRISPR gene-editing system targets IL1RAP for the treatment of low back pain. In some embodiments, the pharmaceutical composition comprising a CRISPR gene-editing system targets IL1RAP for the treatment of degenerative disc disorder. In some embodiments, the pharmaceutical composition comprising a CRISPR gene-editing system targets IL1RAP for the treatment of internal disc disruption.

### 1. Treatment of Musculoskeletal Disorders by Targeting an Interleukin-1 Receptor Gene

In one aspect, methods and pharmaceutical compositions are provided for treating a musculoskeletal disorder using a CRISPR pharmaceutical composition that targets an interleukin-1 receptor gene. In some embodiments, the CRISPR pharmaceutical composition comprises (i) an RNA-guided nuclease or a nucleic acid encoding an RNA-guided nuclease, and (ii) at least one guide RNA (gRNA), or a nucleic acid encoding at least one gRNA, targeting the interleukin-1 receptor gene, where the gRNA specifically binds a target sequence that is adjacent to a protospacer adjacent motif (PAM) sequence for the RNA-guided nuclease. In some embodiments, the CRISPR pharmaceutical composition comprises (i) an mRNA encoding an RNA-guided nuclease, and (ii) at least one gRNA targeting the interleukin-1 receptor gene. In some embodiments, the mRNA encoding the RNA-guided nuclease and the at least one gRNA are packaged in a lipid nanoparticle (LNP). In some embodiments, the musculoskeletal disorder is Rheumatoid Arthritis, Gout, Osteoarthritis, Osteoporosis, Intervertebral disc disease (IVDD), Psoriatic arthritis (PsA), Arthritis, Polymyositis, Proliferative synovitis, Malignant bone neoplasm, Sarcoid Myopathy, Cortex Bone Disorders, Idiopathic Scoliosis, Tendinopathy, Myofibrillar Myopathy, Enthesis-Related Arthritis, Ankylosing spondylitis, Degenerative, polyarthritis, Arthropathy, Osteitis Deformans, Prolapsed Lumbar Disc, Polymyositis Ossificans, Idiopathic Polymyositis, Luft Disease, Adult-onset Still's Disease, Osteoarthrosis Deformans, or Bachet's Disease. In some embodiments, a musculoskeletal disorder listed in FIG. 3 is treated with a CRISPR pharmaceutical composition targeting a gene listed as associated with the disorder in FIG. 3. For example, in some embodiments, Rheumatoid Arthritis is treated with a CRISPR pharmaceutical composition targeting an IL1R1, IL1RAP, IL1R5, IL1R6, IL1R7, or IL1R9 gene.

In some embodiments, the interleukin-1 receptor gene is an IL1RAP gene. In some embodiments, the IL1R1 gene is a human IL1RAP gene and the guide RNA comprises a crRNA sequence selected from SEQ ID NOs: 1040-1203. In some embodiments, the IL1RAP gene is a canine IL1RAP gene and the guide RNA comprises a crRNA sequence selected from SEQ ID NOs: 1204-1271 and SEQ ID NOs: 3421-3490. In some embodiments, the IL1RAP gene is an equine IL1RAP gene and the guide RNA comprises a crRNA sequence selected from SEQ ID NOs: 1272-1348. In some embodiments, the IL1R1 gene is a feline IL1RAP gene and the guide RNA comprises a crRNA sequence selected from SEQ ID NOs: 1349-1424. In some embodiments, editing of the IL1RAP gene results in a knockout of the IL1RAP gene. In some such embodiments, the IL1RAP gene is a human IL1RAP gene and the guide RNA comprises a crRNA sequence selected from SEQ ID NOs: 1040-1122. In some such embodiments, the IL1RAP gene is a canine IL1RAP gene and the guide RNA comprises a crRNA sequence selected from SEQ ID NOs: 1204-1271 and SEQ ID NOS: 3421-3458. In some such embodiments, the IL1RAP gene is an equine IL1RAP gene and the guide RNA comprises a crRNA sequence selected from SEQ ID NOs: 1272-1301. In some such embodiments, the IL1RAP gene is a feline IL1RAP gene and the guide RNA comprises a crRNA sequence selected from SEQ ID NOs: 1349-1378. In some embodiments, editing of the IL1RAP gene results in a gene that expresses a membrane-bound receptor decoy protein. In some such embodiments, the IL1RAP gene is a human IL1RAP gene and the guide RNA comprises a crRNA sequence selected from SEQ ID NOs: 1123-1180 and SEQ ID NOs: 1192-1194. In some such embodiments, the IL1RAP gene is a canine IL1RAP gene and the guide RNA comprises a crRNA sequence selected from SEQ ID NOs: 3459-3485. In some such embodiments, the IL1RAP gene is an equine IL1RAP gene and the guide RNA comprises a crRNA sequence selected from SEQ ID NOs: 1323-1348. In some such embodiments, the IL1RAP gene is a feline IL1RAP gene and the guide RNA comprises a crRNA sequence selected from SEQ ID NOs: 1397-1424. In some embodiments, editing of the IL1RAP gene results in a gene that expresses a soluble receptor decoy protein. In some such embodiments, the IL1RAP gene is a human IL1RAP gene and the guide RNA comprises a crRNA sequence selected from SEQ ID NOs: 1181-1194. In some such embodiments, the IL1RAP gene is a canine IL1RAP gene and the guide RNA comprises a crRNA sequence selected from SEQ ID NOs: 3481-3490 and SEQ ID NOs: 3453-3458. In some such embodiments, the IL1RAP gene is an equine IL1RAP gene and the guide RNA comprises a crRNA sequence selected from SEQ ID NOs: 1302-1322. In some such embodiments, the IL1R1 gene is a feline IL1RAP gene and the guide RNA comprises a crRNA sequence selected from SEQ ID NOs: 1379-1396.

### 2. Treatment of Neoplasia by Targeting an Interleukin-1 Receptor Gene

In In one aspect, methods and pharmaceutical compositions are provided for treating a neoplasia using a CRISPR pharmaceutical composition that targets an interleukin-1 receptor gene. In some embodiments, the CRISPR pharmaceutical composition comprises (i) an RNA-guided nuclease or a nucleic acid encoding an RNA-guided nuclease, and (ii) at least one guide RNA (gRNA), or a nucleic acid encoding at least one gRNA, targeting the interleukin-1 receptor gene, where the gRNA specifically binds a target sequence that is adjacent to a protospacer adjacent motif (PAM) sequence for the RNA-guided nuclease. In some embodiments, the CRISPR pharmaceutical composition comprises (i) an mRNA encoding an RNA-guided nuclease, and (ii) at least one gRNA targeting the interleukin-1 receptor gene. In some embodiments, the mRNA encoding the RNA-guided nuclease and the at least one gRNA are packaged in a lipid nanoparticle (LNP). In some embodiments, the neoplasia is Osteosarcoma, Colon Cancer, Metastasis (General), Lung Cancer, Multiple Myeloma. Breast Cancer, Solid Tumors, Lymphoma, Pancreatic Cancer, Stomach Cancer, Epithelial Ovarian Cancer, Mammary Neoplasms, Oropharyngeal Carcinoma, Renal Cell Carcinoma, Chondrosarcoma, Esophageal Neoplasms, B-Cell Lymphoma, Cutaneous Lymphoma T-Cell, Leukemia, Thyroid Carcinoma, Skin carcinogenesis, Cholectoral Cancer, Glioma, Liver Cancer, Melanoma, Neuroblastoma, Polycystic Ovary Syndrome, Glioblastoma, Prostate Cancer, Cervical Cancer, Ovarian Cancer, Bladder Cancer, Squamous cell carcinoma, Kaposi Sarcoma, Oral Cavity Cancer, Leiomyosarcoma, Malignant Peripheral Nerve Sheath Tumor, or Ewing's Sarcoma. In some embodiments, a neoplasia listed in FIG. 3 is treated with a CRISPR pharmaceutical composition targeting a gene listed as associated with the disorder in FIG. 3. For example, in some embodiments, Osteosarcoma is treated with a CRISPR pharmaceutical composition targeting an IL1R1 gene.

In some embodiments, the interleukin-1 receptor gene is an IL1RAP gene. In some embodiments, the IL1R1 gene is a human IL1RAP gene and the guide RNA comprises a crRNA sequence selected from SEQ ID NOs: 1040-1203. In some embodiments, the IL1RAP gene is a canine IL1RAP gene and the guide RNA comprises a crRNA sequence selected from SEQ ID NOs: 1204-1271 and SEQ ID NOs: 3421-3490. In some embodiments, the IL1RAP gene is an equine IL1RAP gene and the guide RNA comprises a crRNA sequence selected from SEQ ID NOs: 1272-1348. In some embodiments, the IL1R1 gene is a feline IL1RAP gene and the guide RNA comprises a crRNA sequence selected from SEQ ID NOs: 1349-1424. In some embodiments, editing of the IL1RAP gene results in a knockout of the IL1RAP gene. In some such embodiments, the IL1RAP gene is a human IL1RAP gene and the guide RNA comprises a crRNA sequence selected from SEQ ID NOs: 1040-1122. In some such embodiments, the IL1RAP gene is a canine IL1RAP gene and the guide RNA comprises a crRNA sequence selected from SEQ ID NOs: 1204-1271 and SEQ ID NOs: 3421-3458. In some such embodiments, the IL1RAP gene is an equine IL1RAP gene and the guide RNA comprises a crRNA sequence selected from SEQ ID NOs: 1272-1301. In some such embodiments, the IL1RAP gene is a feline IL1RAP gene and the guide RNA comprises a crRNA sequence selected from SEQ ID NOs: 1349-1378. In some embodiments, editing of the IL1RAP gene results in a gene that expresses a membrane-bound receptor decoy protein. In some such embodiments, the IL1RAP gene is a human IL1RAP gene and the guide RNA comprises a crRNA sequence selected from SEQ ID NOs: 1123-1180 and SEQ ID NOs: 1192-1194. In some such embodiments, the IL1RAP gene is a canine IL1RAP gene and the guide RNA comprises a crRNA sequence selected from SEQ ID NOs: 3459-3485. In some such embodiments, the IL1RAP gene is an equine IL1RAP gene and the guide RNA comprises a crRNA sequence selected from SEQ ID NOs: 1323-1348. In some such embodiments, the IL1RAP gene is a feline IL1RAP gene and the guide RNA comprises a crRNA sequence selected from SEQ ID NOs: 1397-1424. In some embodiments, editing of the IL1RAP gene results in a gene that expresses a soluble receptor decoy protein. In some such embodiments, the IL1RAP gene is a human IL1RAP gene and the guide RNA comprises a crRNA sequence selected from SEQ ID NOs: 1181-1194. In some such embodiments, the IL1RAP gene is a canine IL1RAP gene and the guide RNA comprises a crRNA sequence selected from SEQ ID NOs: 3481-3490 and SEQ ID NOs: 3453-3458. In some such embodiments, the IL1RAP gene is an equine IL1RAP gene and the guide RNA comprises a crRNA sequence selected from SEQ ID NOs: 1302-1322. In some such embodiments, the IL1R1 gene is a feline IL1RAP gene and the guide RNA comprises a crRNA sequence selected from SEQ ID NOs: 1379-1396.

### 3. Treatment of Neurological Disorder by Targeting an Interleukin-1 Receptor Gene

In one aspect, methods and pharmaceutical compositions are provided for treating a neurological disorder using a CRISPR pharmaceutical composition that targets an interleukin-1 receptor gene. In some embodiments, the CRISPR pharmaceutical composition comprises (i) an RNA-guided nuclease or a nucleic acid encoding an RNA-guided nuclease, and (ii) at least one guide RNA (gRNA), or a nucleic acid encoding at least one gRNA, targeting the interleukin-1 receptor gene, where the gRNA specifically binds a target sequence that is adjacent to a protospacer adjacent motif (PAM) sequence for the RNA-guided nuclease. In some embodiments, the CRISPR pharmaceutical composition comprises (i) an mRNA encoding an RNA-guided nuclease, and (ii) at least one gRNA targeting the interleukin-1 receptor gene. In some embodiments, the mRNA encoding the RNA-guided nuclease and the at least one gRNA are packaged in a lipid nanoparticle (LNP). In some embodiments, the neurological disorder is Acute Thrombotic Stroke, Epilepsy, Multiple Sclerosis, or Alzheimer's Disease. In some embodiments, a neurological disorder listed in FIG. 3 is treated with a CRISPR pharmaceutical composition targeting a gene listed as associated with the disorder in FIG. 3. For example, in some embodiments, Acute Thrombocytic Stroke is treated with a CRISPR pharmaceutical composition targeting an IL1R1, or an IL1RAP gene.

In some embodiments, the interleukin-1 receptor gene is an IL1RAP gene. In some embodiments, the IL1R1 gene is a human IL1RAP gene and the guide RNA comprises a crRNA sequence selected from SEQ ID NOs: 1040-1203. In some embodiments, the IL1RAP gene is a canine IL1RAP gene and the guide RNA comprises a crRNA sequence selected from SEQ ID NOs: 1204-1271 and SEQ ID NOs: 3421-3490. In some embodiments, the IL1RAP gene is an equine IL1RAP gene and the guide RNA comprises a crRNA sequence selected from SEQ ID NOs: 1272-1348. In some embodiments, the IL1R1 gene is a feline IL1RAP gene and the guide RNA comprises a crRNA sequence selected from SEQ ID NOs: 1349-1424. In some embodiments, editing of the IL1RAP gene results in a knockout of the IL1RAP gene. In some such embodiments, the IL1RAP gene is a human IL1RAP gene and the guide RNA comprises a crRNA sequence selected from SEQ ID NOs: 1040-1122. In some such embodiments, the IL1RAP gene is a canine IL1RAP gene and the guide RNA comprises a crRNA sequence selected from SEQ ID NOs: 1204-1271 and SEQ ID NOs: 3421-3458. In some such embodiments, the IL1RAP gene is an equine IL1RAP gene and the guide RNA comprises a crRNA sequence selected from SEQ ID NOs: 1272-1301. In some such embodiments, the IL1RAP gene is a feline IL1RAP gene and the guide RNA comprises a crRNA sequence selected from SEQ ID NOs: 1349-1378. In some embodiments, editing of the IL1RAP gene results in a gene that expresses a membrane-bound receptor decoy protein. In some such embodiments, the IL1RAP gene is a human IL1RAP gene and the guide RNA comprises a crRNA sequence selected from SEQ ID NOs: 1123-1180 and SEQ ID NOS: 1192-1194. In some such embodiments, the IL1RAP gene is a canine IL1RAP gene and the guide RNA comprises a crRNA sequence selected from SEQ ID NOs: 3459-3485. In some such embodiments, the IL1RAP gene is an equine IL1RAP gene and the guide RNA comprises a crRNA sequence selected from SEQ ID NOs: 1323-1348. In some such embodiments, the IL1RAP gene is a feline IL1RAP gene and the guide RNA comprises a crRNA sequence selected from SEQ ID NOs: 1397-1424. In some embodiments, editing of the IL1RAP gene results in a gene that expresses a soluble receptor decoy protein. In some such embodiments, the IL1RAP gene is a human IL1RAP gene and the guide RNA comprises a crRNA sequence selected from SEQ ID NOs: 1181-1194. In some such embodiments, the IL1RAP gene is a canine IL1RAP gene and the guide RNA comprises a crRNA sequence selected from SEQ ID NOs: 3481-3490 and SEQ ID NOs: 3453-3458. In some such embodiments, the IL1RAP gene is an equine IL1RAP gene and the guide RNA comprises a crRNA sequence selected from SEQ ID NOs: 1302-1322. In some such embodiments, the IL1R1 gene is a feline IL1RAP gene and the guide RNA comprises a crRNA sequence selected from SEQ ID NOs: 1379-1396.

### 4. Treatment of Cardiac Disorder by Targeting an Interleukin-1 Receptor Gene

In one aspect, methods and pharmaceutical compositions are provided for treating a cardiac disorder using a CRISPR pharmaceutical composition that targets an interleukin-1 receptor gene. In some embodiments, the CRISPR pharmaceutical composition comprises (i) an RNA-guided nuclease or a nucleic acid encoding an RNA-guided nuclease, and (ii) at least one guide RNA (gRNA), or a nucleic acid encoding at least one gRNA, targeting the interleukin-1 receptor gene, where the gRNA specifically binds a target sequence that is adjacent to a protospacer adjacent motif (PAM) sequence for the RNA-guided nuclease. In some embodiments, the CRISPR pharmaceutical composition comprises (i) an mRNA encoding an RNA-guided nuclease, and (ii) at least one gRNA targeting the interleukin-1 receptor gene. In some embodiments, the mRNA encoding the RNA-guided nuclease and the at least one gRNA are packaged in a lipid nanoparticle (LNP). In some embodiments, the cardiac disorder is Heart disease including but not limited to Acute Myocardial Infarction, refractory heart failure, arrhythmias, pericarditis, myocarditis, and sepsis-induced cardiomyopathy; Acute Decompensated Heart Failure, Refractory Idiopathic Pericarditis, or Atherosclerosis including but not limited to coronary artery disease, myocardial infarction, and cardiac remodeling. In some embodiments, a cardiac disorder listed in FIG. 3 is treated with a CRISPR pharmaceutical composition targeting a gene listed as associated with the disorder in FIG. 3. For example, in some embodiments, Acute Decompensated Heart Failure is treated with a CRISPR pharmaceutical composition targeting an IL1R1, or an IL1RAP gene.

In some embodiments, the interleukin-1 receptor gene is an IL1RAP gene. In some embodiments, the IL1R1 gene is a human IL1RAP gene and the guide RNA comprises a crRNA sequence selected from SEQ ID NOs: 1040-1203. In some embodiments, the IL1RAP gene is a canine IL1RAP gene and the guide RNA comprises a crRNA sequence selected from SEQ ID NOs: 1204-1271 and SEQ ID NOs: 3421-3490. In some embodiments, the IL1RAP gene is an equine IL1RAP gene and the guide RNA comprises a crRNA sequence selected from SEQ ID NOs: 1272-1348. In some embodiments, the IL1R1 gene is a feline IL1RAP gene and the guide RNA comprises a crRNA sequence selected from SEQ ID NOs: 1349-1424. In some embodiments, editing of the IL1RAP gene results in a knockout of the IL1RAP gene. In some such embodiments, the IL1RAP gene is a human IL1RAP gene and the guide RNA comprises a crRNA sequence selected from SEQ ID NOs: 1040-1122. In some such embodiments, the IL1RAP gene is a canine IL1RAP gene and the guide RNA comprises a crRNA sequence selected from SEQ ID NOs: 1204-1271 and SEQ ID NOs: 3421-3458. In some such embodiments, the IL1RAP gene is an equine IL1RAP gene and the guide RNA comprises a crRNA sequence selected from SEQ ID NOs: 1272-1301. In some such embodiments, the IL1RAP gene is a feline IL1RAP gene and the guide RNA comprises a crRNA sequence selected from SEQ ID NOs: 1349-1378. In some embodiments, editing of the IL1RAP gene results in a gene that expresses a membrane-bound receptor decoy protein. In some such embodiments, the IL1RAP gene is a human IL1RAP gene and the guide RNA comprises a crRNA sequence selected from SEQ ID NOs: 1123-1180 and SEQ ID NOs: 1192-1194. In some such embodiments, the IL1RAP gene is a canine IL1RAP gene and the guide RNA comprises a crRNA sequence selected from SEQ ID NOs: 3459-3485. In some such embodiments, the IL1RAP gene is an equine IL1RAP gene and the guide RNA comprises a crRNA sequence selected from SEQ ID NOs: 1323-1348. In some such embodiments, the IL1RAP gene is a feline IL1RAP gene and the guide RNA comprises a crRNA sequence selected from SEQ ID NOs: 1397-1424. In some embodiments, editing of the IL1RAP gene results in a gene that expresses a soluble receptor decoy protein. In some such embodiments, the IL1RAP gene is a human IL1RAP gene and the guide RNA comprises a crRNA sequence selected from SEQ ID NOs: 1181-1194. In some such embodiments, the IL1RAP gene is a canine IL1RAP gene and the guide RNA comprises a crRNA sequence selected from SEQ ID NOs: 3481-3490 and SEQ ID NOs: 3453-3458. In some such embodiments, the IL1RAP gene is an equine IL1RAP gene and the guide RNA comprises a crRNA sequence selected from SEQ ID NOs: 1302-1322. In some such embodiments, the IL1R1 gene is a feline IL1RAP gene and the guide RNA comprises a crRNA sequence selected from SEQ ID NOs: 1379-1396.

### 5. Treatment of Inflammatory Disorders by Targeting an Interleukin-1 Receptor Gene

In one aspect, methods and pharmaceutical compositions are provided for treating an inflammatory disorder using a CRISPR pharmaceutical composition that targets an interleukin-1 receptor gene. In some embodiments, the CRISPR pharmaceutical composition comprises (i) an RNA-guided nuclease or a nucleic acid encoding an RNA-guided nuclease, and (ii) at least one guide RNA (gRNA), or a nucleic acid encoding at least one gRNA, targeting the interleukin-1 receptor gene, where the gRNA specifically binds a target sequence that is adjacent to a protospacer adjacent motif (PAM) sequence for the RNA-guided nuclease. In some embodiments, the CRISPR pharmaceutical composition comprises (i) an mRNA encoding an RNA-guided nuclease, and (ii) at least one gRNA targeting the interleukin-1 receptor gene. In some embodiments, the mRNA encoding the RNA-guided nuclease and the at least one gRNA are packaged in a lipid nanoparticle (LNP). In some embodiments, the inflammatory disorder is Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), or Fibrosis (general). In some embodiments, an inflammatory disorder listed in FIG. 3 is treated with a CRISPR pharmaceutical composition targeting a gene listed as associated with the disorder in FIG. 3. For example, in some embodiments, an autoinflammatory disease (AID) is treated with a CRISPR pharmaceutical composition targeting an IL1R1, IL1RAP, IL1R5, or an IL1R7 gene.

In some embodiments, the interleukin-1 receptor gene is an IL1RAP gene. In some embodiments, the IL1R1 gene is a human IL1RAP gene and the guide RNA comprises a crRNA sequence selected from SEQ ID NOs: 1040-1203. In some embodiments, the IL1RAP gene is a canine IL1RAP gene and the guide RNA comprises a crRNA sequence selected from SEQ ID NOs: 1204-1271 and SEQ ID NOs: 3421-3490. In some embodiments, the IL1RAP gene is an equine IL1RAP gene and the guide RNA comprises a crRNA sequence selected from SEQ ID NOs: 1272-1348. In some embodiments, the IL1R1 gene is a feline IL1RAP gene and the guide RNA comprises a crRNA sequence selected from SEQ ID NOs: 1349-1424. In some embodiments, editing of the IL1RAP gene results in a knockout of the IL1RAP gene. In some such embodiments, the IL1RAP gene is a human IL1RAP gene and the guide RNA comprises a crRNA sequence selected from SEQ ID NOs: 1040-1122. In some such embodiments, the IL1RAP gene is a canine IL1RAP gene and the guide RNA comprises a crRNA sequence selected from SEQ ID NOs: 1204-1271 and SEQ ID NOs: 3421-3458. In some such embodiments, the IL1RAP gene is an equine IL1RAP gene and the guide RNA comprises a crRNA sequence selected from SEQ ID NOs: 1272-1301. In some such embodiments, the IL1RAP gene is a feline IL1RAP gene and the guide RNA comprises a crRNA sequence selected from SEQ ID NOs: 1349-1378. In some embodiments, editing of the IL1RAP gene results in a gene that expresses a membrane-bound receptor decoy protein. In some such embodiments, the IL1RAP gene is a human IL1RAP gene and the guide RNA comprises a crRNA sequence selected from SEQ ID NOs: 1123-1180 and SEQ ID NOs: 1192-1194. In some such embodiments, the IL1RAP gene is a canine IL1RAP gene and the guide RNA comprises a crRNA sequence selected from SEQ ID NOs: 3459-3485. In some such embodiments, the IL1RAP gene is an equine IL1RAP gene and the guide RNA comprises a crRNA sequence selected from SEQ ID NOs: 1323-1348. In some such embodiments, the IL1RAP gene is a feline IL1RAP gene and the guide RNA comprises a crRNA sequence selected from SEQ ID NOs: 1397-1424. In some embodiments, editing of the IL1RAP gene results in a gene that expresses a soluble receptor decoy protein. In some such embodiments, the IL1RAP gene is a human IL1RAP gene and the guide RNA comprises a crRNA sequence selected from SEQ ID NOs: 1181-1194. In some such embodiments, the IL1RAP gene is a canine IL1RAP gene and the guide RNA comprises a crRNA sequence selected from SEQ ID NOs: 3481-3490 and SEQ ID NOs: 3453-3458. In some such embodiments, the IL1RAP gene is an equine IL1RAP gene and the guide RNA comprises a crRNA sequence selected from SEQ ID NOs: 1302-1322. In some such embodiments, the IL1R1 gene is a feline IL1RAP gene and the guide RNA comprises a crRNA sequence selected from SEQ ID NOs: 1379-1396.

### 6. Treatment of Digestive Disorders by Targeting an Interleukin-1 Receptor Gene

In one aspect, methods and pharmaceutical compositions are provided for treating a digestive disorder using a CRISPR pharmaceutical composition that targets an interleukin-1 receptor gene. In some embodiments, the CRISPR pharmaceutical composition comprises (i) an RNA-guided nuclease or a nucleic acid encoding an RNA-guided nuclease, and (ii) at least one guide RNA (gRNA), or a nucleic acid encoding at least one gRNA, targeting the interleukin-1 receptor gene, where the gRNA specifically binds a target sequence that is adjacent to a protospacer adjacent motif (PAM) sequence for the RNA-guided nuclease. In some embodiments, the CRISPR pharmaceutical composition comprises (i) an mRNA encoding an RNA-guided nuclease, and (ii) at least one gRNA targeting the interleukin-1 receptor gene. In some embodiments, the mRNA encoding the RNA-guided nuclease and the at least one gRNA are packaged in a lipid nanoparticle (LNP). In some embodiments, the digestive disorder is Inflammatory Bowel Disease (IBD), Gastroesophageal reflux disease (GERD), or Non-HP-associated Peptic Ulcer Disease. In some embodiments, a digestive disorder listed in FIG. 3 is treated with a CRISPR pharmaceutical composition targeting a gene listed as associated with the disorder in FIG. 3. For example, in some embodiments, an Inflammatory Bowel Disease (IBD) is treated with a CRISPR pharmaceutical composition targeting an IL1R1, IL1RAP, IL1R5, IL1R6, IL1R7, IL1R8, or an IL1R9 gene.

In some embodiments, the interleukin-1 receptor gene is an IL1RAP gene. In some embodiments, the IL1R1 gene is a human IL1RAP gene and the guide RNA comprises a crRNA sequence selected from SEQ ID NOs: 1040-1203. In some embodiments, the IL1RAP gene is a canine IL1RAP gene and the guide RNA comprises a crRNA sequence selected from SEQ ID NOs: 1204-1271 and SEQ ID NOs: 3421-3490. In some embodiments, the IL1RAP gene is an equine IL1RAP gene and the guide RNA comprises a crRNA sequence selected from SEQ ID NOs: 1272-1348. In some embodiments, the IL1R1 gene is a feline IL1RAP gene and the guide RNA comprises a crRNA sequence selected from SEQ ID NOs: 1349-1424. In some embodiments, editing of the IL1RAP gene results in a knockout of the IL1RAP gene. In some such embodiments, the IL1RAP gene is a human IL1RAP gene and the guide RNA comprises a crRNA sequence selected from SEQ ID NOs: 1040-1122. In some such embodiments, the IL1RAP gene is a canine IL1RAP gene and the guide RNA comprises a crRNA sequence selected from SEQ ID NOs: 1204-1271 and SEQ ID NOs: 3421-3458. In some such embodiments, the IL1RAP gene is an equine IL1RAP gene and the guide RNA comprises a crRNA sequence selected from SEQ ID NOs: 1272-1301. In some such embodiments, the IL1RAP gene is a feline IL1RAP gene and the guide RNA comprises a crRNA sequence selected from SEQ ID NOs: 1349-1378. In some embodiments, editing of the IL1RAP gene results in a gene that expresses a membrane-bound receptor decoy protein. In some such embodiments, the IL1RAP gene is a human IL1RAP gene and the guide RNA comprises a crRNA sequence selected from SEQ ID NOs: 1123-1180 and SEQ ID NOs: 1192-1194. In some such embodiments, the IL1RAP gene is a canine IL1RAP gene and the guide RNA comprises a crRNA sequence selected from SEQ ID NOs: 3459-3485. In some such embodiments, the IL1RAP gene is an equine IL1RAP gene and the guide RNA comprises a crRNA sequence selected from SEQ ID NOs: 1323-1348. In some such embodiments, the IL1RAP gene is a feline IL1RAP gene and the guide RNA comprises a crRNA sequence selected from SEQ ID NOs: 1397-1424. In some embodiments, editing of the IL1RAP gene results in a gene that expresses a soluble receptor decoy protein. In some such embodiments, the IL1RAP gene is a human IL1RAP gene and the guide RNA comprises a crRNA sequence selected from SEQ ID NOs: 1181-1194. In some such embodiments, the IL1RAP gene is a canine IL1RAP gene and the guide RNA comprises a crRNA sequence selected from SEQ ID NOs: 3481-3490 and SEQ ID NOs: 3453-3458. In some such embodiments, the IL1RAP gene is an equine IL1RAP gene and the guide RNA comprises a crRNA sequence selected from SEQ ID NOs: 1302-1322. In some such embodiments, the IL1R1 gene is a feline IL1RAP gene and the guide RNA comprises a crRNA sequence selected from SEQ ID NOs: 1379-1396.

### 7. Treatment of Respiratory Disorders by Targeting an Interleukin-1 Receptor Gene

In one aspect, methods and pharmaceutical compositions are provided for treating a respiratory disorder using a CRISPR pharmaceutical composition that targets an interleukin-1 receptor gene. In some embodiments, the CRISPR pharmaceutical composition comprises (i) an RNA-guided nuclease or a nucleic acid encoding an RNA-guided nuclease, and (ii) at least one guide RNA (gRNA), or a nucleic acid encoding at least one gRNA, targeting the interleukin-1 receptor gene, where the gRNA specifically binds a target sequence that is adjacent to a protospacer adjacent motif (PAM) sequence for the RNA-guided nuclease. In some embodiments, the CRISPR pharmaceutical composition comprises (i) an mRNA encoding an RNA-guided nuclease, and (ii) at least one gRNA targeting the interleukin-1 receptor gene. In some embodiments, the mRNA encoding the RNA-guided nuclease and the at least one gRNA are packaged in a lipid nanoparticle (LNP). In some embodiments, the respiratory disorder is Asthma, or Pulmonary Fibrosis. In some embodiments, a respiratory disorder listed in FIG. 3 is treated with a CRISPR pharmaceutical composition targeting a gene listed as associated with the disorder in FIG. 3. For example, in some embodiments, Asthma is treated with a CRISPR pharmaceutical composition targeting an IL1R1, or IL1RAP gene.

In some embodiments, the interleukin-1 receptor gene is an IL1RAP gene. In some embodiments, the IL1R1 gene is a human IL1RAP gene and the guide RNA comprises a crRNA sequence selected from SEQ ID NOs: 1040-1203. In some embodiments, the IL1RAP gene is a canine IL1RAP gene and the guide RNA comprises a crRNA sequence selected from SEQ ID NOs: 1204-1271 and SEQ ID NOs: 3421-3490. In some embodiments, the IL1RAP gene is an equine IL1RAP gene and the guide RNA comprises a crRNA sequence selected from SEQ ID NOs: 1272-1348. In some embodiments, the IL1R1 gene is a feline IL1RAP gene and the guide RNA comprises a crRNA sequence selected from SEQ ID NOs: 1349-1424. In some embodiments, editing of the IL1RAP gene results in a knockout of the IL1RAP gene. In some such embodiments, the IL1RAP gene is a human IL1RAP gene and the guide RNA comprises a crRNA sequence selected from SEQ ID NOs: 1040-1122. In some such embodiments, the IL1RAP gene is a canine IL1RAP gene and the guide RNA comprises a crRNA sequence selected from SEQ ID NOs: 1204-1271 and SEQ ID NOs: 3421-3458. In some such embodiments, the IL1RAP gene is an equine IL1RAP gene and the guide RNA comprises a crRNA sequence selected from SEQ ID NOs: 1272-1301. In some such embodiments, the IL1RAP gene is a feline IL1RAP gene and the guide RNA comprises a crRNA sequence selected from SEQ ID NOs: 1349-1378. In some embodiments, editing of the IL1RAP gene results in a gene that expresses a membrane-bound receptor decoy protein. In some such embodiments, the IL1RAP gene is a human IL1RAP gene and the guide RNA comprises a crRNA sequence selected from SEQ ID NOs: 1123-1180 and SEQ ID NOS: 1192-1194. In some such embodiments, the IL1RAP gene is a canine IL1RAP gene and the guide RNA comprises a crRNA sequence selected from SEQ ID NOs: 3459-3485. In some such embodiments, the IL1RAP gene is an equine IL1RAP gene and the guide RNA comprises a crRNA sequence selected from SEQ ID NOs: 1323-1348. In some such embodiments, the IL1RAP gene is a feline IL1RAP gene and the guide RNA comprises a crRNA sequence selected from SEQ ID NOs: 1397-1424. In some embodiments, editing of the IL1RAP gene results in a gene that expresses a soluble receptor decoy protein. In some such embodiments, the IL1RAP gene is a human IL1RAP gene and the guide RNA comprises a crRNA sequence selected from SEQ ID NOs: 1181-1194. In some such embodiments, the IL1RAP gene is a canine IL1RAP gene and the guide RNA comprises a crRNA sequence selected from SEQ ID NOs: 3481-3490 and SEQ ID NOs: 3453-3458. In some such embodiments, the IL1RAP gene is an equine IL1RAP gene and the guide RNA comprises a crRNA sequence selected from SEQ ID NOs: 1302-1322. In some such embodiments, the IL1R1 gene is a feline IL1RAP gene and the guide RNA comprises a crRNA sequence selected from SEQ ID NOs: 1379-1396.

### 8. Treatment of Renal, Metabolic, or Ophthalmic Disorders by Targeting an Interleukin-1 Receptor Gene

In one aspect, methods and pharmaceutical compositions are provided for treating a renal, metabolic, or ophthalmic disorder using a CRISPR pharmaceutical composition that targets an interleukin-1 receptor gene. In some embodiments, the CRISPR pharmaceutical composition comprises (i) an RNA-guided nuclease or a nucleic acid encoding an RNA-guided nuclease, and (ii) at least one guide RNA (gRNA), or a nucleic acid encoding at least one gRNA, targeting the interleukin-1 receptor gene, where the gRNA specifically binds a target sequence that is adjacent to a protospacer adjacent motif (PAM) sequence for the RNA-guided nuclease. In some embodiments, the CRISPR pharmaceutical composition comprises (i) an mRNA encoding an RNA-guided nuclease, and (ii) at least one gRNA targeting the interleukin-1 receptor gene. In some embodiments, the mRNA encoding the RNA-guided nuclease and the at least one gRNA are packaged in a lipid nanoparticle (LNP). In some embodiments, the renal, metabolic, or ophthalmic disorder is Chronic Kidney Disease, Type 2 Diabetes, or Eye Diseases including but not limited to Uveitis, Scleritis, Sjogren asthenia, and dry eye. In some embodiments, a renal, metabolic, or ophthalmic disorder listed in FIG. 3 is treated with a CRISPR pharmaceutical composition targeting a gene listed as associated with the disorder in FIG. 3. For example, in some embodiments, Chronic Kidney disease is treated with a CRISPR pharmaceutical composition targeting an IL1R1, or IL1RAP gene.

In some embodiments, the interleukin-1 receptor gene is an IL1RAP gene. In some embodiments, the IL1R1 gene is a human IL1RAP gene and the guide RNA comprises a crRNA sequence selected from SEQ ID NOs: 1040-1203. In some embodiments, the IL1RAP gene is a canine IL1RAP gene and the guide RNA comprises a crRNA sequence selected from SEQ ID NOs: 1204-1271 and SEQ ID NOs: 3421-3490. In some embodiments, the IL1RAP gene is an equine IL1RAP gene and the guide RNA comprises a crRNA sequence selected from SEQ ID NOs: 1272-1348. In some embodiments, the IL1R1 gene is a feline IL1RAP gene and the guide RNA comprises a crRNA sequence selected from SEQ ID NOs: 1349-1424. In some embodiments, editing of the IL1RAP gene results in a knockout of the IL1RAP gene. In some such embodiments, the IL1RAP gene is a human IL1RAP gene and the guide RNA comprises a crRNA sequence selected from SEQ ID NOs: 1040-1122. In some such embodiments, the IL1RAP gene is a canine IL1RAP gene and the guide RNA comprises a crRNA sequence selected from SEQ ID NOs: 1204-1271 and SEQ ID NOs: 3421-3458. In some such embodiments, the IL1RAP gene is an equine IL1RAP gene and the guide RNA comprises a crRNA sequence selected from SEQ ID NOs: 1272-1301. In some such embodiments, the IL1RAP gene is a feline IL1RAP gene and the guide RNA comprises a crRNA sequence selected from SEQ ID NOs: 1349-1378. In some embodiments, editing of the IL1RAP gene results in a gene that expresses a membrane-bound receptor decoy protein. In some such embodiments, the IL1RAP gene is a human IL1RAP gene and the guide RNA comprises a crRNA sequence selected from SEQ ID NOs: 1123-1180 and SEQ ID NOs: 1192-1194. In some such embodiments, the IL1RAP gene is a canine IL1RAP gene and the guide RNA comprises a crRNA sequence selected from SEQ ID NOs: 3459-3485. In some such embodiments, the IL1RAP gene is an equine IL1RAP gene and the guide RNA comprises a crRNA sequence selected from SEQ ID NOs: 1323-1348. In some such embodiments, the IL1RAP gene is a feline IL1RAP gene and the guide RNA comprises a crRNA sequence selected from SEQ ID NOs: 1397-1424. In some embodiments, editing of the IL1RAP gene results in a gene that expresses a soluble receptor decoy protein. In some such embodiments, the IL1RAP gene is a human IL1RAP gene and the guide RNA comprises a crRNA sequence selected from SEQ ID NOs: 1181-1194. In some such embodiments, the IL1RAP gene is a canine IL1RAP gene and the guide RNA comprises a crRNA sequence selected from SEQ ID NOs: 3481-3490 and SEQ ID NOs: 3453-3458. In some such embodiments, the IL1RAP gene is an equine IL1RAP gene and the guide RNA comprises a crRNA sequence selected from SEQ ID NOs: 1302-1322. In some such embodiments, the IL1R1 gene is a feline IL1RAP gene and the guide RNA comprises a crRNA sequence selected from SEQ ID NOs: 1379-1396.

### C. TGFBR1

In some embodiments, the pharmaceutical composition comprising a CRISPR gene-editing system targets TGFBR1. In some embodiments, the CRISPR gene-editing system targeting TGFBR1 is delivered to a mammalian cell via viral vector. In some embodiments, the CRISPR gene-editing system targeting TGFBR1 is delivered to a mammalian cell via an AAV vector. In some embodiments, the CRISPR gene-editing system targeting TGFBR1 is delivered to a mammalian cell via a lentiviral vector. In some embodiments, the CRISPR gene-editing system targeting TGFBR1 is delivered to a mammalian cell via a lipid nanoparticle. In some embodiments, the CRISPR gene-editing system targeting TGFBR1 is delivered to a mammalian cell via a virus-like particle. In some embodiments, the CRISPR gene-editing system targeting TGFBR1 is delivered to a mammalian cell via a liposome. In some embodiments, the CRISPR gene-editing system targeting TGFBR1 is delivered to a mammalian cell via a lipid nanocrystal.

In various embodiments, the pharmaceutical composition comprising a CRISPR gene-editing system that targets the TGFBR1 gene is used in a method of treating a mammal in need thereof. In some embodiments, the method treats one or more joint disease or illness or one or more back or spine conditions or disorders. In some embodiments, the method treats one or more musculoskeletal diseases, conditions, and illnesses, including, but not limited to, Loeys-Dietz Syndrome, osteoarthritis, Marfan syndrome, aortic aneurysm (e.g., familial thoracic 3 aortic aneurysm), craniofacial abnormalities, and combinations thereof. In other embodiments, the method treats neoplastic diseases, conditions, and illnesses, including, but not limited to, pancreatic cancer, multiple self-healing squamous epithelioma (Ferguson-Smith disease), gastrointestinal stromal tumors (GIST), hereditary nonpolyposis colorectal cancer (Lynch Syndrome), metastatic colorectal carcinoma, bone neoplasms, anaplastic carcinoma, spindle-cell carcinoma, lung neoplasms, brain neoplasms, and combinations thereof.

In some embodiments, the pharmaceutical composition comprising a CRISPR gene-editing system targets TGFBR1 for the treatment of a joint disease or illness. In some embodiments, the pharmaceutical composition comprising a CRISPR gene-editing system targets TGFBR1 for the treatment of osteoarthritis. In some embodiments, the pharmaceutical composition comprising a CRISPR gene-editing system targets TGFBR1 for the treatment of gout.

In some embodiments, the pharmaceutical composition comprising a CRISPR gene-editing system targets TGFBR1 for the treatment of a musculoskeletal disorder.

In some embodiments, the pharmaceutical composition comprising a CRISPR gene-editing system targets TGFBR1 for the treatment of a back or spine condition or disorder. In some embodiments, the pharmaceutical composition comprising a CRISPR gene-editing system targets TGFBR1 for the treatment of low back pain. In some embodiments, the pharmaceutical composition comprising a CRISPR gene-editing system targets TGFBR1 for the treatment of degenerative disc disorder. In some embodiments, the pharmaceutical composition comprising a CRISPR gene-editing system targets TGFBR1 for the treatment of internal disc disruption.

### 1. Treatment of Musculoskeletal Disorders by Targeting an TGF-Beta Receptor Gene

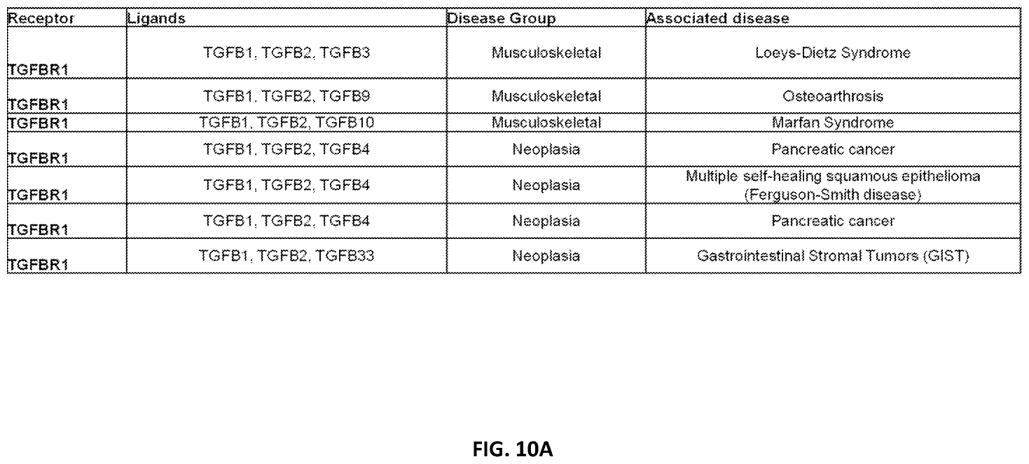

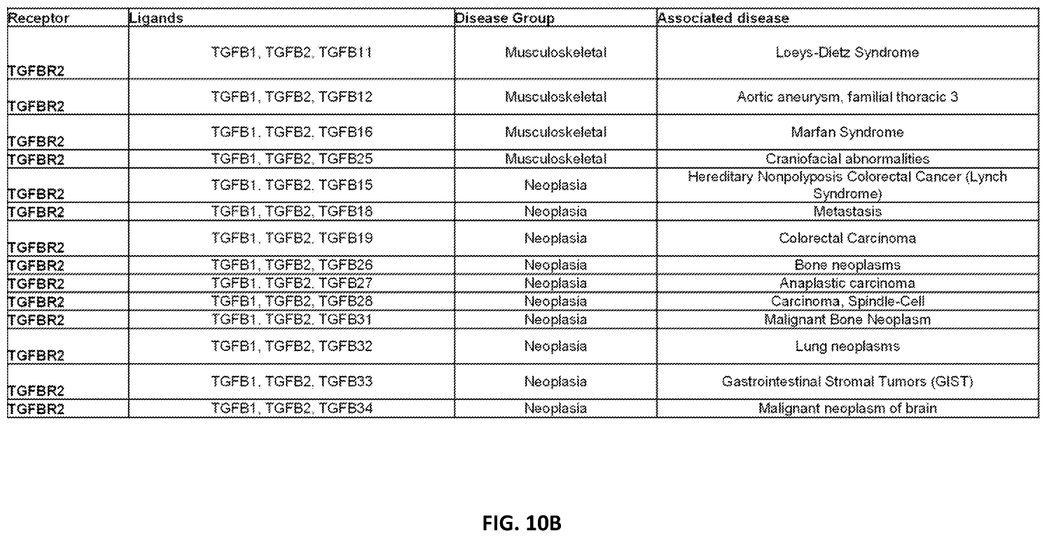

In one aspect, methods and pharmaceutical compositions are provided for treating a musculoskeletal disorder using a CRISPR pharmaceutical composition that targets a Transforming Growth Factor Beta Receptor gene. In some embodiments, the CRISPR pharmaceutical composition comprises (i) an RNA-guided nuclease or a nucleic acid encoding an RNA-guided nuclease, and (ii) at least one guide RNA (gRNA), or a nucleic acid encoding at least one gRNA, targeting the Transforming Growth Factor Beta Receptor gene, where the gRNA specifically binds a target sequence that is adjacent to a protospacer adjacent motif (PAM) sequence for the RNA-guided nuclease. In some embodiments, the CRISPR pharmaceutical composition comprises (i) an mRNA encoding an RNA-guided nuclease, and (ii) at least one gRNA targeting the Transforming Growth Factor Beta Receptor gene. In some embodiments, the mRNA encoding the RNA-guided nuclease and the at least one gRNA are packaged in a lipid nanoparticle (LNP). In some embodiments, the musculoskeletal disorder is Loeys-Dietz Syndrome, Osteoarthrosis, Marfan Syndrome, Aortic aneurysm (familial thoracic 3), or Craniofacial abnormalities. In some embodiments, a musculoskeletal disorder listed in FIG. 10 is treated with a CRISPR pharmaceutical composition targeting a gene listed as associated with the disorder in FIG. 10. For example, in some embodiments, Osteoarthritis is treated with a CRISPR pharmaceutical composition targeting a TGFBR1 or TGFBR2 gene.

In some embodiments, the transforming growth factor beta receptor gene is an TGFBR1 gene. In some embodiments, the TGFBR1 gene is a human TGFBR1 gene and the guide RNA comprises a crRNA sequence selected from SEQ ID NOs: 1425-1546. In some embodiments, editing of the TGFBR1 gene results in a knockout of the TGFBR1 gene. In some such embodiments, the TGFBR1 gene is a human TGFBR1 gene and the guide RNA comprises a crRNA sequence selected from SEQ ID NOS: 1425-1454. In some embodiments, editing of the TGFBR1 gene results in a gene that expresses a membrane-bound receptor decoy protein. In some such embodiments, the TGFBR1 gene is a human TGFBR1 gene and the guide RNA comprises a crRNA sequence selected from SEQ ID NOs: 1455-1534. In some embodiments, editing of the TGFBR1 gene results in a gene that expresses a soluble receptor decoy protein. In some such embodiments, the TGFBR1 gene is a human TGFBR1 gene and the guide RNA comprises a crRNA sequence selected from SEQ ID NOs: 1535-1544.

### 2. Treatment of Neoplasia by Targeting a TGF-Beta Receptor Gene

In one aspect, methods and pharmaceutical compositions are provided for treating a neoplasia using a CRISPR pharmaceutical composition that targets a Transforming Growth Factor Beta Receptor gene. In some embodiments, the CRISPR pharmaceutical composition comprises (i) an RNA-guided nuclease or a nucleic acid encoding an RNA-guided nuclease, and (ii) at least one guide RNA (gRNA), or a nucleic acid encoding at least one gRNA, targeting the Transforming Growth Factor Beta Receptor gene, where the gRNA specifically binds a target sequence that is adjacent to a protospacer adjacent motif (PAM) sequence for the RNA-guided nuclease. In some embodiments, the CRISPR pharmaceutical composition comprises (i) an mRNA encoding an RNA-guided nuclease, and (ii) at least one gRNA targeting the Transforming Growth Factor Beta Receptor gene. In some embodiments, the mRNA encoding the RNA-guided nuclease and the at least one gRNA are packaged in a lipid nanoparticle (LNP). In some embodiments, the neoplasia is Pancreatic cancer, Multiple self-healing squamous epithelioma (Ferguson-Smith disease), Pancreatic cancer, Gastrointestinal Stromal Tumors (GIST), Hereditary Nonpolyposis Colorectal Cancer (Lynch Syndrome), Metastasis, Colorectal Carcinoma, Bone neoplasms, Anaplastic carcinoma, Spindle-Cell carcinoma, Malignant Bone Neoplasm, Lung neoplasms, or Malignant brain neoplasm. In some embodiments, a neoplasia listed in FIG. 10 is treated with a CRISPR pharmaceutical composition targeting a gene listed as associated with the disorder in FIG. 10. For example, in some embodiments, Pancreatic cancer is treated with a CRISPR pharmaceutical composition targeting a TGFBR2 gene.

In some embodiments, the transforming growth factor beta receptor gene is an TGFBR1 gene. In some embodiments, the TGFBR1 gene is a human TGFBR1 gene and the guide RNA comprises a crRNA sequence selected from SEQ ID NOS: 1425-1544. In some embodiments, editing of the TGFBR1 gene results in a knockout of the TGFBR1 gene. In some such embodiments, the TGFBR1 gene is a human TGFBR1 gene and the guide RNA comprises a crRNA sequence selected from SEQ ID NOS: 1425-1454. In some embodiments, editing of the TGFBR1 gene results in a gene that expresses a membrane-bound receptor decoy protein. In some such embodiments, the TGFBR1 gene is a human TGFBR1 gene and the guide RNA comprises a crRNA sequence selected from SEQ ID NOS: 1455-1534. In some embodiments, editing of the TGFBR1 gene results in a gene that expresses a soluble receptor decoy protein. In some such embodiments, the TGFBR1 gene is a human TGFBR1 gene and the guide RNA comprises a crRNA sequence selected from SEQ ID NOS: 1535-1544.

### D. TGFBR2

In some embodiments, the pharmaceutical composition comprising a CRISPR gene-editing system targets TGFBR2. In some embodiments, the CRISPR gene-editing system targeting TGFBR2 is delivered to a mammalian cell via viral vector. In some embodiments, the CRISPR gene-editing system targeting TGFBR2 is delivered to a mammalian cell via an AAV vector. In some embodiments, the CRISPR gene-editing system targeting TGFBR2 is delivered to a mammalian cell via a lentiviral vector. In some embodiments, the CRISPR gene-editing system targeting TGFBR2 is delivered to a mammalian cell via a lipid nanoparticle. In some embodiments, the CRISPR gene-editing system targeting TGFBR2 is delivered to a mammalian cell via a virus-like particle. In some embodiments, the CRISPR gene-editing system targeting TGFBR2 is delivered to a mammalian cell via a liposome. In some embodiments, the CRISPR gene-editing system targeting TGFBR2 is delivered to a mammalian cell via a lipid nanocrystal.

In various embodiments, the pharmaceutical composition comprising a CRISPR gene-editing system that targets the TGFBR2 gene is used in a method of treating a mammal in need thereof. In some embodiments, the method treats one or more joint disease or illness or one or more back or spine conditions or disorders. In some embodiments, the method treats one or more musculoskeletal diseases, conditions, and illnesses, including, but not limited to, Loeys-Dietz Syndrome, osteoarthritis, Marfan syndrome, aortic aneurysm (e.g., familial thoracic 3 aortic aneurysm), craniofacial abnormalities, and combinations thereof. In other embodiments, the method treats neoplastic diseases, conditions, and illnesses, including, but not limited to, pancreatic cancer, multiple self-healing squamous epithelioma (Ferguson-Smith disease), gastrointestinal stromal tumors (GIST), hereditary nonpolyposis colorectal cancer (Lynch Syndrome), metastatic colorectal carcinoma, bone neoplasms, anaplastic carcinoma, spindle-cell carcinoma, lung neoplasms, brain neoplasms, and combinations thereof.

In some embodiments, the pharmaceutical composition comprising a CRISPR gene-editing system targets TGFBR2 for the treatment of a joint disease or illness. In some embodiments, the pharmaceutical composition comprising a CRISPR gene-editing system targets TGFBR2 for the treatment of osteoarthritis. In some embodiments, the pharmaceutical composition comprising a CRISPR gene-editing system targets TGFBR2 for the treatment of gout.

In some embodiments, the pharmaceutical composition comprising a CRISPR gene-editing system targets TGFBR2 for the treatment of a musculoskeletal disorder.

In some embodiments, the pharmaceutical composition comprising a CRISPR gene-editing system targets TGFBR2 for the treatment of a back or spine condition or disorder. In some embodiments, the pharmaceutical composition comprising a CRISPR gene-editing system targets TGFBR2 for the treatment of low back pain. In some embodiments, the pharmaceutical composition comprising a CRISPR gene-editing system targets TGFBR2 for the treatment of degenerative disc disorder. In some embodiments, the pharmaceutical composition comprising a CRISPR gene-editing system targets TGFBR2 for the treatment of internal disc disruption.

### 1. Treatment of Musculoskeletal Disorders by Targeting an TGF-Beta Receptor Gene

In one aspect, methods and pharmaceutical compositions are provided for treating a musculoskeletal disorder using a CRISPR pharmaceutical composition that targets a Transforming Growth Factor Beta Receptor gene. In some embodiments, the CRISPR pharmaceutical composition comprises (i) an RNA-guided nuclease or a nucleic acid encoding an RNA-guided nuclease, and (ii) at least one guide RNA (gRNA), or a nucleic acid encoding at least one gRNA, targeting the Transforming Growth Factor Beta Receptor gene, where the gRNA specifically binds a target sequence that is adjacent to a protospacer adjacent motif (PAM) sequence for the RNA-guided nuclease. In some embodiments, the CRISPR pharmaceutical composition comprises (i) an mRNA encoding an RNA-guided nuclease, and (ii) at least one gRNA targeting the Transforming Growth Factor Beta Receptor gene. In some embodiments, the mRNA encoding the RNA-guided nuclease and the at least one gRNA are packaged in a lipid nanoparticle (LNP). In some embodiments, the musculoskeletal disorder is Loeys-Dietz Syndrome, Osteoarthrosis, Marfan Syndrome, Aortic aneurysm (familial thoracic 3), or Craniofacial abnormalities. In some embodiments, a musculoskeletal disorder listed in FIG. 10 is treated with a CRISPR pharmaceutical composition targeting a gene listed as associated with the disorder in FIG. 10. For example, in some embodiments, Osteoarthritis is treated with a CRISPR pharmaceutical composition targeting a TGFBR1 or TGFBR2 gene.

In some embodiments, the transforming growth factor beta receptor gene is an TGFBR2 gene. In some embodiments, the TGFBR2 gene is a human TGFBR2 gene and the guide RNA comprises a crRNA sequence selected from SEQ ID NOs: 1547-1734. In some embodiments, editing of the TGFBR2 gene results in a knockout of the TGFBR2 gene. In some such embodiments, the TGFBR2 gene is a human TGFBR2 gene and the guide RNA comprises a crRNA sequence selected from SEQ ID NOs: 1547-1580. In some embodiments, editing of the TGFBR2 gene results in a gene that expresses a membrane-bound receptor decoy protein. In some such embodiments, the TGFBR2 gene is a human TGFBR2 gene and the guide RNA comprises a crRNA sequence selected from SEQ ID NOs: 1581-1724. In some embodiments, editing of the TGFBR2 gene results in a gene that expresses a soluble receptor decoy protein. In some such embodiments, the TGFBR2 gene is a human TGFBR2 gene and the guide RNA comprises a crRNA sequence selected from SEQ ID NOs: 1725-1734.

### 2. Treatment of Neoplasia by Targeting a TGF-Beta Receptor Gene

In one aspect, methods and pharmaceutical compositions are provided for treating a neoplasia using a CRISPR pharmaceutical composition that targets a Transforming Growth Factor Beta Receptor gene. In some embodiments, the CRISPR pharmaceutical composition comprises (i) an RNA-guided nuclease or a nucleic acid encoding an RNA-guided nuclease, and (ii) at least one guide RNA (gRNA), or a nucleic acid encoding at least one gRNA, targeting the Transforming Growth Factor Beta Receptor gene, where the gRNA specifically binds a target sequence that is adjacent to a protospacer adjacent motif (PAM) sequence for the RNA-guided nuclease. In some embodiments, the CRISPR pharmaceutical composition comprises (i) an mRNA encoding an RNA-guided nuclease, and (ii) at least one gRNA targeting the Transforming Growth Factor Beta Receptor gene. In some embodiments, the mRNA encoding the RNA-guided nuclease and the at least one gRNA are packaged in a lipid nanoparticle (LNP). In some embodiments, the neoplasia is Pancreatic cancer, Multiple self-healing squamous epithelioma (Ferguson-Smith disease), Pancreatic cancer, Gastrointestinal Stromal Tumors (GIST), Hereditary Nonpolyposis Colorectal Cancer (Lynch Syndrome), Metastasis, Colorectal Carcinoma, Bone neoplasms, Anaplastic carcinoma, Spindle-Cell carcinoma, Malignant Bone Neoplasm, Lung neoplasms, or Malignant brain neoplasm. In some embodiments, a neoplasia listed in FIG. 10 is treated with a CRISPR pharmaceutical composition targeting a gene listed as associated with the disorder in FIG. 10. For example, in some embodiments, Pancreatic cancer is treated with a CRISPR pharmaceutical composition targeting a TGFBR2 gene.

In some embodiments, the transforming growth factor beta receptor gene is an TGFBR2 gene. In some embodiments, the TGFBR2 gene is a human TGFBR2 gene and the guide RNA comprises a crRNA sequence selected from SEQ ID NOs: 1547-1734. In some embodiments, editing of the TGFBR2 gene results in a knockout of the TGFBR2 gene. In some such embodiments, the TGFBR2 gene is a human TGFBR2 gene and the guide RNA comprises a crRNA sequence selected from SEQ ID NOs: 1547-1580. In some embodiments, editing of the TGFBR2 gene results in a gene that expresses a membrane-bound receptor decoy protein. In some such embodiments, the TGFBR2 gene is a human TGFBR2 gene and the guide RNA comprises a crRNA sequence selected from SEQ ID NOs: 1581-1724. In some embodiments, editing of the TGFBR2 gene results in a gene that expresses a soluble receptor decoy protein. In some such embodiments, the TGFBR2 gene is a human TGFBR2 gene and the guide RNA comprises a crRNA sequence selected from SEQ ID NOs: 1725-1734.

### E. IL6R

In some embodiments, the pharmaceutical composition comprising a CRISPR gene-editing system targets IL6R. In some embodiments, the CRISPR gene-editing system targeting IL6R is delivered to a mammalian cell via viral vector. In some embodiments, the CRISPR gene-editing system targeting IL6R is delivered to a mammalian cell via an AAV vector. In some embodiments, the CRISPR gene-editing system targeting IL6R is delivered to a mammalian cell via a lentiviral vector. In some embodiments, the CRISPR gene-editing system targeting IL6R is delivered to a mammalian cell via a lipid nanoparticle. In some embodiments, the CRISPR gene-editing system targeting IL6R is delivered to a mammalian cell via a virus-like particle. In some embodiments, the CRISPR gene-editing system targeting IL6R is delivered to a mammalian cell via a liposome. In some embodiments, the CRISPR gene-editing system targeting IL6R is delivered to a mammalian cell via a lipid nanocrystal.

In various embodiments, the pharmaceutical composition comprising a CRISPR gene-editing system that targets the IL6R gene is used in a method of treating a mammal in need thereof. In some embodiments, the method treats one or more joint disease or illness or one or more back or spine conditions or disorders. In some embodiments, the method treats one or more musculoskeletal diseases, conditions, and illnesses, including, but not limited to, rheumatoid arthritis, gout, osteoarthritis, osteoporosis, intervertebral disc disease (IVDD), psoriatic arthritis, arthritis, polymyositis, proliferative synovitis, bone neoplasms, sarcoid myopathy, cortex bone disorders, idiopathic scoliosis, tendinopathy, myofibrillar myopathy, enthesis-related arthritis, ankylosing spondylitis, degenerative polyarthritis, arthropathy, osteitis deformans, prolapsed lumbar disc, polymyositis ossificans, idiopathic polymyositis, Luft Disease, adult-onset Still's Disease, osteoarthritis deformans, Bachet's Disease, and combinations thereof. In other embodiments, use of the system treats one or more neoplastic diseases, conditions, and illnesses, including, but not limited to, osteosarcoma, multiple myeloma, lymphoma (e.g., B-cell or cutaneous T-cell lymphoma), leukemia, thyroid carcinoma, glioma, renal cell carcinoma, chondrosarcoma, glioblastoma, melanoma, neuroblastoma, polycystic ovary syndrome, Kaposi sarcoma, squamous cell carcinoma, leiomyosarcoma, malignant peripheral nerve sheath tumor, Ewing's sarcoma, esophageal neoplasms, colon cancer, lung cancer, breast cancer, pancreatic cancer, stomach cancer, epithelial ovarian cancer, cholectoral cancer, liver cancer, skin cancer, prostate cancer, cervical cancer, ovarian cancer, bladder cancer, oral cavity cancer, and combinations thereof. In other embodiments, the method treats one or more inflammatory diseases, conditions, and illnesses, including, but not limited to, autoinflammatory disease (AID), cryopyrin-associated periodic syndrome (CAPS), systemic lupus erythematosus (SLE), inflammatory bowel disease (IBD) and combinations thereof. In some embodiments, the method treats one or more cardiac diseases, conditions, and illnesses, including, but not limited to, myocardial infarction, heart failure (e.g., refractory or acute decompensated heart failure), arrythmias, pericarditis (e.g., refractory idiopathic pericarditis), myocarditis, sepsis-induced cardiomyopathy, atherosclerosis, coronary artery disease and combinations thereof. In some embodiments, the method treats one or more neurological diseases, conditions, and illnesses, including, but not limited to, acute thrombotic stroke, epilepsy, multiple sclerosis, Alzheimer's Disease and combinations thereof. In some embodiments, the method treats one or more ophthalmic diseases, conditions, and illnesses, including, but not limited to, uveitis, scleritis, Sjogren asthenia, dry eye and combinations thereof. In some embodiments, the method treats one or more diseases, conditions, and illnesses, including, but not limited to, chronic kidney disease, Type 2 diabetes, gastroesophageal reflux disease (GERD), non-HP-associated peptic ulcer disease, and pulmonary fibrosis.

In some embodiments, the pharmaceutical composition comprising a CRISPR gene-editing system targets IL6R for the treatment of a joint disease or illness. In some embodiments, the pharmaceutical composition comprising a CRISPR gene-editing system targets IL6R for the treatment of osteoarthritis. In some embodiments, the pharmaceutical composition comprising a CRISPR gene-editing system targets IL6R for the treatment of gout.

In some embodiments, the pharmaceutical composition comprising a CRISPR gene-editing system targets IL6R for the treatment of a musculoskeletal disorder.

In some embodiments, the pharmaceutical composition comprising a CRISPR gene-editing system targets IL6R for the treatment of a back or spine condition or disorder. In some embodiments, the pharmaceutical composition comprising a CRISPR gene-editing system targets IL6R for the treatment of low back pain. In some embodiments, the pharmaceutical composition comprising a CRISPR gene-editing system targets IL6R for the treatment of degenerative disc disorder. In some embodiments, the pharmaceutical composition comprising a CRISPR gene-editing system targets IL6R for the treatment of internal disc disruption.

### 1. Treatment of Musculoskeletal Disorders by Targeting an Interleukin-6 Receptor Gene

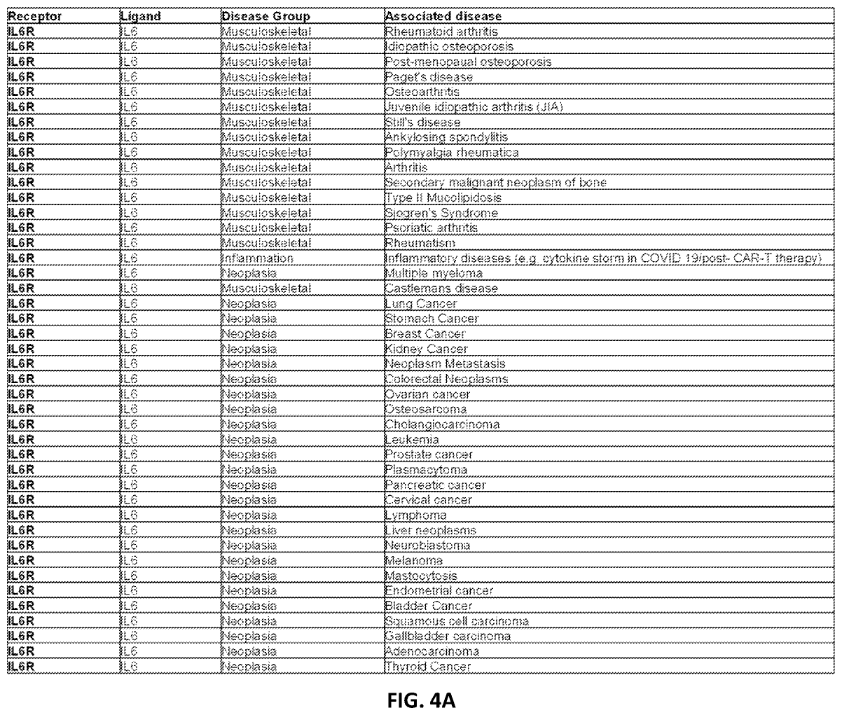

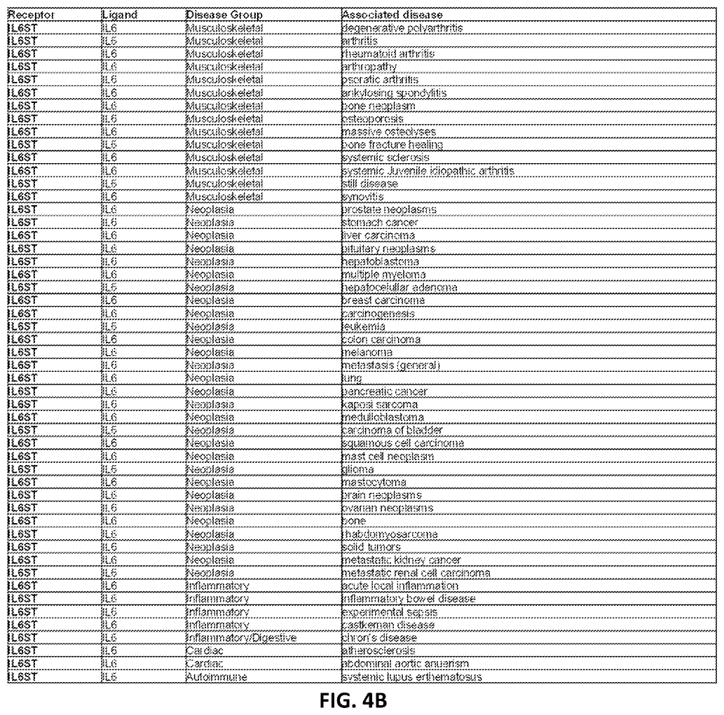

In one aspect, methods and pharmaceutical compositions are provided for treating a musculoskeletal disorder using a CRISPR pharmaceutical composition that targets an interleukin-6 receptor gene. In some embodiments, the CRISPR pharmaceutical composition comprises (i) an RNA-guided nuclease or a nucleic acid encoding an RNA-guided nuclease, and (ii) at least one guide RNA (gRNA), or a nucleic acid encoding at least one gRNA, targeting the interleukin-6 receptor gene, where the gRNA specifically binds a target sequence that is adjacent to a protospacer adjacent motif (PAM) sequence for the RNA-guided nuclease. In some embodiments, the CRISPR pharmaceutical composition comprises (i) an mRNA encoding an RNA-guided nuclease, and (ii) at least one gRNA targeting the interleukin-6 receptor gene. In some embodiments, the mRNA encoding the RNA-guided nuclease and the at least one gRNA are packaged in a lipid nanoparticle (LNP). In some embodiments, the musculoskeletal disorder is Rheumatoid arthritis, Idiopathic osteoporosis, Post-menopausal osteoporosis, Paget's disease, Osteoarthritis, Juvenile idiopathic arthritis (JIA), Still's disease, Ankylosing spondylitis, Polymyalgia rheumatica, Arthritis, Secondary malignant neoplasm of bone, Type II, Mucolipidosis, Sjogren's Syndrome, Psoriatic arthritis, Rheumatism, Castleman's disease, Degenerative Polyarthritis, Arthropathy, Bone neoplasm, Osteoporosis, Massive osteolysis, Bone fracture healing, Systemic sclerosis, Systemic Juvenile Idiopathic, Arthritis, or Synovitis. In some embodiments, a musculoskeletal disorder listed in FIG. 4 is treated with a CRISPR pharmaceutical composition targeting a gene listed as associated with the disorder in FIG. 4. For example, in some embodiments, Rheumatoid Arthritis is treated with a CRISPR pharmaceutical composition targeting an IL6R or IL6ST gene.

In some embodiments, the interleukin-6 receptor gene is an IL6R gene. In some embodiments, the IL6R gene is a human IL6R gene and the guide RNA comprises a crRNA sequence selected from SEQ ID NOs: 1746-1968. In some embodiments, editing of the IL6R gene results in a knockout of the IL6R gene. In some such embodiments, the IL6R gene is a human IL6R gene and the guide RNA comprises a crRNA sequence selected from SEQ ID SEQ ID NOs: 1746-1907. In some embodiments, editing of the IL6R gene results in a gene that expresses a soluble receptor decoy protein. In some such embodiments, the IL6R gene is a human IL6R gene and the guide RNA comprises a crRNA sequence selected from SEQ ID SEQ ID NOs: 1835-1920.

### 2. Treatment of Neoplasia by Targeting an Interleukin-6 Receptor Gene

In one aspect, methods and pharmaceutical compositions are provided for treating a neoplasia using a CRISPR pharmaceutical composition that targets an interleukin-6 receptor gene. In some embodiments, the CRISPR pharmaceutical composition comprises (i) an RNA-guided nuclease or a nucleic acid encoding an RNA-guided nuclease, and (ii) at least one guide RNA (gRNA), or a nucleic acid encoding at least one gRNA, targeting the interleukin-6 receptor gene, where the gRNA specifically binds a target sequence that is adjacent to a protospacer adjacent motif (PAM) sequence for the RNA-guided nuclease. In some embodiments, the CRISPR pharmaceutical composition comprises (i) an mRNA encoding an RNA-guided nuclease, and (ii) at least one gRNA targeting the interleukin-6 receptor gene. In some embodiments, the mRNA encoding the RNA-guided nuclease and the at least one gRNA are packaged in a lipid nanoparticle (LNP). In some embodiments, the neoplasia is Multiple myeloma, Lung Cancer, Stomach Cancer, Breast Cancer, Kidney Cancer, Neoplasm Metastasis, Colorectal Neoplasms, Ovarian cancer, Osteosarcoma, Cholangiocarcinoma, Leukemia, Prostate cancer, Plasmacytoma, Pancreatic cancer, Cervical cancer, Lymphoma, Liver neoplasms, Neuroblastoma, Melanoma, Mastocytosis, Endometrial cancer, Bladder Cancer, Squamous cell carcinoma, Gallbladder carcinoma, Adenocarcinoma, Thyroid Cancer, prostate neoplasms, stomach cancer, liver carcinoma, pituitary neoplasms, hepatoblastoma, multiple myeloma, hepatocellular adenoma, breast carcinoma, carcinogenesis, leukemia, colon carcinoma, melanoma, metastasis (general), lung cancer, pancreatic cancer, Kaposi sarcoma, medulloblastoma, carcinoma of bladder, squamous cell carcinoma, mast cell neoplasm, glioma, mastocytoma, brain neoplasms, ovarian neoplasms, bone neoplasm, rhabdomyosarcoma, solid tumors, metastatic kidney cancer, or metastatic renal cell carcinoma. In some embodiments, a neoplasia listed in FIG. 4 is treated with a CRISPR pharmaceutical composition targeting a gene listed as associated with the disorder in FIG. 4. For example, in some embodiments, Prostate Cancer is treated with a CRISPR pharmaceutical composition targeting an IL6R or IL6ST gene.

In some embodiments, the interleukin-6 receptor gene is an IL6R gene. In some embodiments, the IL6R gene is a human IL6R gene and the guide RNA comprises a crRNA sequence selected from SEQ ID NOs: 1746-1968. In some embodiments, editing of the IL6R gene results in a knockout of the IL6R gene. In some such embodiments, the IL6R gene is a human IL6R gene and the guide RNA comprises a crRNA sequence selected from SEQ ID SEQ ID NOs: 1746-1907. In some embodiments, editing of the IL6R gene results in a gene that expresses a soluble receptor decoy protein. In some such embodiments, the IL6R gene is a human IL6R gene and the guide RNA comprises a crRNA sequence selected from SEQ ID SEQ ID NOs: 1835-1920.

### 3. Treatment of Inflammatory Disorders by Targeting an Interleukin-6 Receptor Gene

In one aspect, methods and pharmaceutical compositions are provided for treating an inflammatory disorder using a CRISPR pharmaceutical composition that targets an interleukin-6 receptor gene. In some embodiments, the CRISPR pharmaceutical composition comprises (i) an RNA-guided nuclease or a nucleic acid encoding an RNA-guided nuclease, and (ii) at least one guide RNA (gRNA), or a nucleic acid encoding at least one gRNA, targeting the interleukin-6 receptor gene, where the gRNA specifically binds a target sequence that is adjacent to a protospacer adjacent motif (PAM) sequence for the RNA-guided nuclease. In some embodiments, the CRISPR pharmaceutical composition comprises (i) an mRNA encoding an RNA-guided nuclease, and (ii) at least one gRNA targeting the interleukin-6 receptor gene. In some embodiments, the mRNA encoding the RNA-guided nuclease and the at least one gRNA are packaged in a lipid nanoparticle (LNP). In some embodiments, the inflammatory disorder is an Inflammatory disease (e.g. cytokine storm in COVID 19 or post-CAR-T therapy), acute local inflammation, inflammatory bowel disease, Crohn's disease, Experimental sepsis, or Castleman's disease.

In some embodiments, the interleukin-6 receptor gene is an IL6R gene. In some embodiments, the IL6R gene is a human IL6R gene and the guide RNA comprises a crRNA sequence selected from SEQ ID NOs: 1746-1968. In some embodiments, editing of the IL6R gene results in a knockout of the IL6R gene. In some such embodiments, the IL6R gene is a human IL6R gene and the guide RNA comprises a crRNA sequence selected from SEQ ID SEQ ID NOs: 1746-1907. In some embodiments, editing of the IL6R gene results in a gene that expresses a soluble receptor decoy protein. In some such embodiments, the IL6R gene is a human IL6R gene and the guide RNA comprises a crRNA sequence selected from SEQ ID SEQ ID NOs: 1835-1920.

### 4. Treatment of Cardiac Disorder by Targeting an Interleukin-6 Receptor Gene

In one aspect, methods and pharmaceutical compositions are provided for treating a cardiac disorder using a CRISPR pharmaceutical composition that targets an interleukin-6 receptor gene. In some embodiments, the CRISPR pharmaceutical composition comprises (i) an RNA-guided nuclease or a nucleic acid encoding an RNA-guided nuclease, and (ii) at least one guide RNA (gRNA), or a nucleic acid encoding at least one gRNA, targeting the interleukin-6 receptor gene, where the gRNA specifically binds a target sequence that is adjacent to a protospacer adjacent motif (PAM) sequence for the RNA-guided nuclease. In some embodiments, the CRISPR pharmaceutical composition comprises (i) an mRNA encoding an RNA-guided nuclease, and (ii) at least one gRNA targeting the interleukin-6 receptor gene. In some embodiments, the mRNA encoding the RNA-guided nuclease and the at least one gRNA are packaged in a lipid nanoparticle (LNP). In some embodiments, the cardiac disorder is atherosclerosis, or abdominal aortic aneurism. In some embodiments, a cardiac disorder listed in FIG. 4 is treated with a CRISPR pharmaceutical composition targeting a gene listed as associated with the disorder in FIG. 4. For example, in some embodiments, atherosclerosis is treated with a CRISPR pharmaceutical composition targeting an IL6R or IL6ST gene.

In some embodiments, the interleukin-6 receptor gene is an IL6R gene. In some embodiments, the IL6R gene is a human IL6R gene and the guide RNA comprises a crRNA sequence selected from SEQ ID NOs: 1746-1968. In some embodiments, editing of the IL6R gene results in a knockout of the IL6R gene. In some such embodiments, the IL6R gene is a human IL6R gene and the guide RNA comprises a crRNA sequence selected from SEQ ID SEQ ID NOs: 1746-1907. In some embodiments, editing of the IL6R gene results in a gene that expresses a soluble receptor decoy protein. In some such embodiments, the IL6R gene is a human IL6R gene and the guide RNA comprises a crRNA sequence selected from SEQ ID SEQ ID NOs: 1835-1920.

### 5. Treatment of Autoimmune Disorder by Targeting an Interleukin-6 Receptor Gene

In one aspect, methods and pharmaceutical compositions are provided for treating an autoimmune disorder using a CRISPR pharmaceutical composition that targets an interleukin-6 receptor gene. In some embodiments, the CRISPR pharmaceutical composition comprises (i) an RNA-guided nuclease or a nucleic acid encoding an RNA-guided nuclease, and (ii) at least one guide RNA (gRNA), or a nucleic acid encoding at least one gRNA, targeting the interleukin-6 receptor gene, where the gRNA specifically binds a target sequence that is adjacent to a protospacer adjacent motif (PAM) sequence for the RNA-guided nuclease. In some embodiments, the CRISPR pharmaceutical composition comprises (i) an mRNA encoding an RNA-guided nuclease, and (ii) at least one gRNA targeting the interleukin-6 receptor gene. In some embodiments, the mRNA encoding the RNA-guided nuclease and the at least one gRNA are packaged in a lipid nanoparticle (LNP). In some embodiments, the autoimmune disorder is Systemic Lupus Erythematosus. In some embodiments, an autoimmune disorder listed in FIG. 4 is treated with a CRISPR pharmaceutical composition targeting a gene listed as associated with the disorder in FIG. 4. For example, in some embodiments, Systemic Lupus Erythematosus is treated with a CRISPR pharmaceutical composition targeting an IL6R or IL6ST gene.

In some embodiments, the interleukin-6 receptor gene is an IL6R gene. In some embodiments, the IL6R gene is a human IL6R gene and the guide RNA comprises a crRNA sequence selected from SEQ ID NOs: 1746-1968. In some embodiments, editing of the IL6R gene results in a knockout of the IL6R gene. In some such embodiments, the IL6R gene is a human IL6R gene and the guide RNA comprises a crRNA sequence selected from SEQ ID SEQ ID NOs: 1746-1907. In some embodiments, editing of the IL6R gene results in a gene that expresses a soluble receptor decoy protein. In some such embodiments, the IL6R gene is a human IL6R gene and the guide RNA comprises a crRNA sequence selected from SEQ ID SEQ ID NOs: 1835-1920.

### F. IL6ST

In some embodiments, the pharmaceutical composition comprising a CRISPR gene-editing system targets IL6ST. In some embodiments, the CRISPR gene-editing system targeting IL6ST is delivered to a mammalian cell via viral vector. In some embodiments, the CRISPR gene-editing system targeting IL6ST is delivered to a mammalian cell via an AAV vector. In some embodiments, the CRISPR gene-editing system targeting IL6ST is delivered to a mammalian cell via a lentiviral vector. In some embodiments, the CRISPR gene-editing system targeting IL6ST is delivered to a mammalian cell via a lipid nanoparticle. In some embodiments, the CRISPR gene-editing system targeting IL6ST is delivered to a mammalian cell via a virus-like particle. In some embodiments, the CRISPR gene-editing system targeting IL6ST is delivered to a mammalian cell via a liposome. In some embodiments, the CRISPR gene-editing system targeting IL6ST is delivered to a mammalian cell via a lipid nanocrystal.

In various embodiments, the pharmaceutical composition comprising a CRISPR gene-editing system that targets the IL6ST gene is used in a method of treating a mammal in need thereof. In some embodiments, the method treats one or more joint disease or illness or one or more back or spine conditions or disorders. In some embodiments, the method treats one or more musculoskeletal diseases, conditions, and illnesses, including, but not limited to, rheumatoid arthritis, gout, osteoarthritis, osteoporosis, intervertebral disc disease (IVDD), psoriatic arthritis, arthritis, polymyositis, proliferative synovitis, bone neoplasms, sarcoid myopathy, cortex bone disorders, idiopathic scoliosis, tendinopathy, myofibrillar myopathy, enthesis-related arthritis, ankylosing spondylitis, degenerative polyarthritis, arthropathy, osteitis deformans, prolapsed lumbar disc, polymyositis ossificans, idiopathic polymyositis, Luft Disease, adult-onset Still's Disease, osteoarthritis deformans, Bachet's Disease, and combinations thereof. In other embodiments, use of the system treats one or more neoplastic diseases, conditions, and illnesses, including, but not limited to, osteosarcoma, multiple myeloma, lymphoma (e.g., B-cell or cutaneous T-cell lymphoma), leukemia, thyroid carcinoma, glioma, renal cell carcinoma, chondrosarcoma, glioblastoma, melanoma, neuroblastoma, polycystic ovary syndrome, Kaposi sarcoma, squamous cell carcinoma, leiomyosarcoma, malignant peripheral nerve sheath tumor, Ewing's sarcoma, esophageal neoplasms, colon cancer, lung cancer, breast cancer, pancreatic cancer, stomach cancer, epithelial ovarian cancer, cholectoral cancer, liver cancer, skin cancer, prostate cancer, cervical cancer, ovarian cancer, bladder cancer, oral cavity cancer, and combinations thereof. In other embodiments, the method treats one or more inflammatory diseases, conditions, and illnesses, including, but not limited to, autoinflammatory disease (AID), cryopyrin-associated periodic syndrome (CAPS), systemic lupus erythematosus (SLE), inflammatory bowel disease (IBD) and combinations thereof. In some embodiments, the method treats one or more cardiac diseases, conditions, and illnesses, including, but not limited to, myocardial infarction, heart failure (e.g., refractory or acute decompensated heart failure), arrythmias, pericarditis (e.g., refractory idiopathic pericarditis), myocarditis, sepsis-induced cardiomyopathy, atherosclerosis, coronary artery disease and combinations thereof. In some embodiments, the method treats one or more neurological diseases, conditions, and illnesses, including, but not limited to, acute thrombotic stroke, epilepsy, multiple sclerosis, Alzheimer's Disease and combinations thereof. In some embodiments, the method treats one or more ophthalmic diseases, conditions, and illnesses, including, but not limited to, uveitis, scleritis, Sjogren asthenia, dry eye and combinations thereof. In some embodiments, the method treats one or more diseases, conditions, and illnesses, including, but not limited to, chronic kidney disease, Type 2 diabetes, gastroesophageal reflux disease (GERD), non-HP-associated peptic ulcer disease, and pulmonary fibrosis.

In some embodiments, the pharmaceutical composition comprising a CRISPR gene-editing system targets IL6ST for the treatment of a joint disease or illness. In some embodiments, the pharmaceutical composition comprising a CRISPR gene-editing system targets IL6ST for the treatment of osteoarthritis. In some embodiments, the pharmaceutical composition comprising a CRISPR gene-editing system targets IL6ST for the treatment of gout.

In some embodiments, the pharmaceutical composition comprising a CRISPR gene-editing system targets IL6ST for the treatment of a musculoskeletal disorder.

In some embodiments, the pharmaceutical composition comprising a CRISPR gene-editing system targets IL6ST for the treatment of a back or spine condition or disorder. In some embodiments, the pharmaceutical composition comprising a CRISPR gene-editing system targets IL6ST for the treatment of low back pain. In some embodiments, the pharmaceutical composition comprising a CRISPR gene-editing system targets IL6ST for the treatment of degenerative disc disorder. In some embodiments, the pharmaceutical composition comprising a CRISPR gene-editing system targets IL6ST for the treatment of internal disc disruption.

### 1. Treatment of Musculoskeletal Disorders by Targeting an Interleukin-6 Receptor Gene

In one aspect, methods and pharmaceutical compositions are provided for treating a musculoskeletal disorder using a CRISPR pharmaceutical composition that targets an interleukin-6 receptor gene. In some embodiments, the CRISPR pharmaceutical composition comprises (i) an RNA-guided nuclease or a nucleic acid encoding an RNA-guided nuclease, and (ii) at least one guide RNA (gRNA), or a nucleic acid encoding at least one gRNA, targeting the interleukin-6 receptor gene, where the gRNA specifically binds a target sequence that is adjacent to a protospacer adjacent motif (PAM) sequence for the RNA-guided nuclease. In some embodiments, the CRISPR pharmaceutical composition comprises (i) an mRNA encoding an RNA-guided nuclease, and (ii) at least one gRNA targeting the interleukin-6 receptor gene. In some embodiments, the mRNA encoding the RNA-guided nuclease and the at least one gRNA are packaged in a lipid nanoparticle (LNP). In some embodiments, the musculoskeletal disorder is Rheumatoid arthritis, Idiopathic osteoporosis, Post-menopausal osteoporosis, Paget's disease, Osteoarthritis, Juvenile idiopathic arthritis (JIA), Still's disease, Ankylosing spondylitis, Polymyalgia rheumatica, Arthritis, Secondary malignant neoplasm of bone, Type II, Mucolipidosis, Sjogren's Syndrome, Psoriatic arthritis, Rheumatism, Castleman's disease, Degenerative Polyarthritis, Arthropathy, Bone neoplasm, Osteoporosis, Massive osteolysis, Bone fracture healing, Systemic sclerosis, Systemic Juvenile Idiopathic, Arthritis, or Synovitis. In some embodiments, a musculoskeletal disorder listed in FIG. 4 is treated with a CRISPR pharmaceutical composition targeting a gene listed as associated with the disorder in FIG. 4. For example, in some embodiments, Rheumatoid Arthritis is treated with a CRISPR pharmaceutical composition targeting an IL6R or IL6ST gene.

In some embodiments, the interleukin-6 receptor gene is an IL6ST gene. In some embodiments, the IL6ST gene is a human IL6ST gene and the guide RNA comprises a crRNA sequence selected from SEQ ID NOs: 1969-2178. In some embodiments, editing of the IL6ST gene results in a knockout of the IL6ST gene. In some such embodiments, the IL6ST gene is a human IL6ST gene and the guide RNA comprises a crRNA sequence selected from SEQ ID NOs: 1969-2037. In some embodiments, editing of the IL6ST gene results in a gene that expresses a membrane-bound receptor decoy protein. In some such embodiments, the IL6ST gene is a human IL6ST gene and the guide RNA comprises a crRNA sequence selected from SEQ ID NOs: 2038-2102. In some embodiments, editing of the IL6ST gene results in a gene that expresses a soluble receptor decoy protein. In some such embodiments, the IL6ST gene is a human IL6ST gene and the guide RNA comprises a crRNA sequence selected from SEQ ID NOs: 2103-2176.

### 2. Treatment of Neoplasia by Targeting an Interleukin-6 Receptor Gene

In one aspect, methods and pharmaceutical compositions are provided for treating a neoplasia using a CRISPR pharmaceutical composition that targets an interleukin-6 receptor gene. In some embodiments, the CRISPR pharmaceutical composition comprises (i) an RNA-guided nuclease or a nucleic acid encoding an RNA-guided nuclease, and (ii) at least one guide RNA (gRNA), or a nucleic acid encoding at least one gRNA, targeting the interleukin-6 receptor gene, where the gRNA specifically binds a target sequence that is adjacent to a protospacer adjacent motif (PAM) sequence for the RNA-guided nuclease. In some embodiments, the CRISPR pharmaceutical composition comprises (i) an mRNA encoding an RNA-guided nuclease, and (ii) at least one gRNA targeting the interleukin-6 receptor gene. In some embodiments, the mRNA encoding the RNA-guided nuclease and the at least one gRNA are packaged in a lipid nanoparticle (LNP). In some embodiments, the neoplasia is Multiple myeloma, Lung Cancer, Stomach Cancer, Breast Cancer, Kidney Cancer, Neoplasm Metastasis, Colorectal Neoplasms, Ovarian cancer, Osteosarcoma, Cholangiocarcinoma, Leukemia, Prostate cancer, Plasmacytoma, Pancreatic cancer, Cervical cancer, Lymphoma, Liver neoplasms, Neuroblastoma, Melanoma, Mastocytosis, Endometrial cancer, Bladder Cancer, Squamous cell carcinoma, Gallbladder carcinoma, Adenocarcinoma, Thyroid Cancer, prostate neoplasms, stomach cancer, liver carcinoma, pituitary neoplasms, hepatoblastoma, multiple myeloma, hepatocellular adenoma, breast carcinoma, carcinogenesis, leukemia, colon carcinoma, melanoma, metastasis (general), lung cancer, pancreatic cancer, Kaposi sarcoma, medulloblastoma, carcinoma of bladder, squamous cell carcinoma, mast cell neoplasm, glioma, mastocytoma, brain neoplasms, ovarian neoplasms, bone neoplasm, rhabdomyosarcoma, solid tumors, metastatic kidney cancer, or metastatic renal cell carcinoma. In some embodiments, a neoplasia listed in FIG. 4 is treated with a CRISPR pharmaceutical composition targeting a gene listed as associated with the disorder in FIG. 4. For example, in some embodiments, Prostate Cancer is treated with a CRISPR pharmaceutical composition targeting an IL6R or IL6ST gene.

In some embodiments, the interleukin-6 receptor gene is an IL6ST gene. In some embodiments, the IL6ST gene is a human IL6ST gene and the guide RNA comprises a crRNA sequence selected from SEQ ID NOs: 1969-2178. In some embodiments, editing of the IL6ST gene results in a knockout of the IL6ST gene. In some such embodiments, the IL6ST gene is a human IL6ST gene and the guide RNA comprises a crRNA sequence selected from SEQ ID NOs: 1969-2037. In some embodiments, editing of the IL6ST gene results in a gene that expresses a membrane-bound receptor decoy protein. In some such embodiments, the IL6ST gene is a human IL6ST gene and the guide RNA comprises a crRNA sequence selected from SEQ ID NOs: 2038-2102. In some embodiments, editing of the IL6ST gene results in a gene that expresses a soluble receptor decoy protein. In some such embodiments, the IL6ST gene is a human IL6ST gene and the guide RNA comprises a crRNA sequence selected from SEQ ID NOs: 2103-2176.

### 3. Treatment of Inflammatory Disorders by Targeting an Interleukin-6 Receptor Gene

In one aspect, methods and pharmaceutical compositions are provided for treating an inflammatory disorder using a CRISPR pharmaceutical composition that targets an interleukin-6 receptor gene. In some embodiments, the CRISPR pharmaceutical composition comprises (i) an RNA-guided nuclease or a nucleic acid encoding an RNA-guided nuclease, and (ii) at least one guide RNA (gRNA), or a nucleic acid encoding at least one gRNA, targeting the interleukin-6 receptor gene, where the gRNA specifically binds a target sequence that is adjacent to a protospacer adjacent motif (PAM) sequence for the RNA-guided nuclease. In some embodiments, the CRISPR pharmaceutical composition comprises (i) an mRNA encoding an RNA-guided nuclease, and (ii) at least one gRNA targeting the interleukin-6 receptor gene. In some embodiments, the mRNA encoding the RNA-guided nuclease and the at least one gRNA are packaged in a lipid nanoparticle (LNP). In some embodiments, the inflammatory disorder is an Inflammatory disease (e.g. cytokine storm in COVID 19 or post-CAR-T therapy), acute local inflammation, inflammatory bowel disease, Crohn's disease, Experimental sepsis, or Castleman's disease.

In some embodiments, the interleukin-6 receptor gene is an IL6ST gene. In some embodiments, the IL6ST gene is a human IL6ST gene and the guide RNA comprises a crRNA sequence selected from SEQ ID NOs: 1969-2178. In some embodiments, editing of the IL6ST gene results in a knockout of the IL6ST gene. In some such embodiments, the IL6ST gene is a human IL6ST gene and the guide RNA comprises a crRNA sequence selected from SEQ ID NOs: 1969-2037. In some embodiments, editing of the IL6ST gene results in a gene that expresses a membrane-bound receptor decoy protein. In some such embodiments, the IL6ST gene is a human IL6ST gene and the guide RNA comprises a crRNA sequence selected from SEQ ID NOs: 2038-2102. In some embodiments, editing of the IL6ST gene results in a gene that expresses a soluble receptor decoy protein. In some such embodiments, the IL6ST gene is a human IL6ST gene and the guide RNA comprises a crRNA sequence selected from SEQ ID NOs: 2103-2176.

### 4. Treatment of Cardiac Disorder by Targeting an Interleukin-6 Receptor Gene

In one aspect, methods and pharmaceutical compositions are provided for treating a cardiac disorder using a CRISPR pharmaceutical composition that targets an interleukin-6 receptor gene. In some embodiments, the CRISPR pharmaceutical composition comprises (i) an RNA-guided nuclease or a nucleic acid encoding an RNA-guided nuclease, and (ii) at least one guide RNA (gRNA), or a nucleic acid encoding at least one gRNA, targeting the interleukin-6 receptor gene, where the gRNA specifically binds a target sequence that is adjacent to a protospacer adjacent motif (PAM) sequence for the RNA-guided nuclease. In some embodiments, the CRISPR pharmaceutical composition comprises (i) an mRNA encoding an RNA-guided nuclease, and (ii) at least one gRNA targeting the interleukin-6 receptor gene. In some embodiments, the mRNA encoding the RNA-guided nuclease and the at least one gRNA are packaged in a lipid nanoparticle (LNP). In some embodiments, the cardiac disorder is atherosclerosis, or abdominal aortic aneurism. In some embodiments, a cardiac disorder listed in FIG. 4 is treated with a CRISPR pharmaceutical composition targeting a gene listed as associated with the disorder in FIG. 4. For example, in some embodiments, atherosclerosis is treated with a CRISPR pharmaceutical composition targeting an IL6R or IL6ST gene.

In some embodiments, the interleukin-6 receptor gene is an IL6ST gene. In some embodiments, the IL6ST gene is a human IL6ST gene and the guide RNA comprises a crRNA sequence selected from SEQ ID NOs: 1969-2178. In some embodiments, editing of the IL6ST gene results in a knockout of the IL6ST gene. In some such embodiments, the IL6ST gene is a human IL6ST gene and the guide RNA comprises a crRNA sequence selected from SEQ ID NOs: 1969-2037. In some embodiments, editing of the IL6ST gene results in a gene that expresses a membrane-bound receptor decoy protein. In some such embodiments, the IL6ST gene is a human IL6ST gene and the guide RNA comprises a crRNA sequence selected from SEQ ID NOs: 2038-2102. In some embodiments, editing of the IL6ST gene results in a gene that expresses a soluble receptor decoy protein. In some such embodiments, the IL6ST gene is a human IL6ST gene and the guide RNA comprises a crRNA sequence selected from SEQ ID NOs: 2103-2176.

### 5. Treatment of Autoimmune Disorder by Targeting an Interleukin-6 Receptor Gene

In one aspect, methods and pharmaceutical compositions are provided for treating an autoimmune disorder using a CRISPR pharmaceutical composition that targets an interleukin-6 receptor gene. In some embodiments, the CRISPR pharmaceutical composition comprises (i) an RNA-guided nuclease or a nucleic acid encoding an RNA-guided nuclease, and (ii) at least one guide RNA (gRNA), or a nucleic acid encoding at least one gRNA, targeting the interleukin-6 receptor gene, where the gRNA specifically binds a target sequence that is adjacent to a protospacer adjacent motif (PAM) sequence for the RNA-guided nuclease. In some embodiments, the CRISPR pharmaceutical composition comprises (i) an mRNA encoding an RNA-guided nuclease, and (ii) at least one gRNA targeting the interleukin-6 receptor gene. In some embodiments, the mRNA encoding the RNA-guided nuclease and the at least one gRNA are packaged in a lipid nanoparticle (LNP). In some embodiments, the autoimmune disorder is Systemic Lupus Erythematosus. In some embodiments, an autoimmune disorder listed in FIG. 4 is treated with a CRISPR pharmaceutical composition targeting a gene listed as associated with the disorder in FIG. 4. For example, in some embodiments, Systemic Lupus Erythematosus is treated with a CRISPR pharmaceutical composition targeting an IL6R or IL6ST gene.

In some embodiments, the interleukin-6 receptor gene is an IL6ST gene. In some embodiments, the IL6ST gene is a human IL6ST gene and the guide RNA comprises a crRNA sequence selected from SEQ ID NOs: 1969-2178. In some embodiments, editing of the IL6ST gene results in a knockout of the IL6ST gene. In some such embodiments, the IL6ST gene is a human IL6ST gene and the guide RNA comprises a crRNA sequence selected from SEQ ID NOs: 1969-2037. In some embodiments, editing of the IL6ST gene results in a gene that expresses a membrane-bound receptor decoy protein. In some such embodiments, the IL6ST gene is a human IL6ST gene and the guide RNA comprises a crRNA sequence selected from SEQ ID NOs: 2038-2102. In some embodiments, editing of the IL6ST gene results in a gene that expresses a soluble receptor decoy protein. In some such embodiments, the IL6ST gene is a human IL6ST gene and the guide RNA comprises a crRNA sequence selected from SEQ ID NOs: 2103-2176.

### G. TNFRSF1A

In some embodiments, the pharmaceutical composition comprising a CRISPR gene-editing system targets TNFRSF1A. In some embodiments, the CRISPR gene-editing system targeting TNFRSF1A is delivered to a mammalian cell via viral vector. In some embodiments, the CRISPR gene-editing system targeting TNFRSF1A is delivered to a mammalian cell via an AAV vector. In some embodiments, the CRISPR gene-editing system targeting TNFRSF1A is delivered to a mammalian cell via a lentiviral vector. In some embodiments, the CRISPR gene-editing system targeting TNFRSF1A is delivered to a mammalian cell via a lipid nanoparticle. In some embodiments, the CRISPR gene-editing system targeting TNFRSF1A is delivered to a mammalian cell via a virus-like particle. In some embodiments, the CRISPR gene-editing system targeting TNFRSF1A is delivered to a mammalian cell via a liposome. In some embodiments, the CRISPR gene-editing system targeting TNFRSF1A is delivered to a mammalian cell via a lipid nanocrystal.

In various embodiments, the pharmaceutical composition comprising a CRISPR gene-editing system that targets the TNFRSF1A gene is used in a method of treating a mammal in need thereof. In some embodiments, the method treats one or more joint disease or illness or one or more back or spine conditions or disorders. In some embodiments, the method treats one or more musculoskeletal diseases, conditions, and illnesses, including, but not limited to, rheumatoid arthritis, gout, osteoarthritis, osteoporosis, intervertebral disc disease (IVDD), psoriatic arthritis, arthritis, polymyositis, proliferative synovitis, bone neoplasms, sarcoid myopathy, cortex bone disorders, idiopathic scoliosis, tendinopathy, myofibrillar myopathy, enthesis-related arthritis, ankylosing spondylitis, degenerative polyarthritis, arthropathy, osteitis deformans, prolapsed lumbar disc, polymyositis ossificans, idiopathic polymyositis, Luft Disease, adult-onset Still's Disease, osteoarthritis deformans, Bachet's Disease, and combinations thereof. In other embodiments, use of the system treats one or more neoplastic diseases, conditions, and illnesses, including, but not limited to, osteosarcoma, multiple myeloma, lymphoma (e.g., B-cell or cutaneous T-cell lymphoma), leukemia, thyroid carcinoma, glioma, renal cell carcinoma, chondrosarcoma, glioblastoma, melanoma, neuroblastoma, polycystic ovary syndrome, Kaposi sarcoma, squamous cell carcinoma, leiomyosarcoma, malignant peripheral nerve sheath tumor, Ewing's sarcoma, esophageal neoplasms, colon cancer, lung cancer, breast cancer, pancreatic cancer, stomach cancer, epithelial ovarian cancer, cholectoral cancer, liver cancer, skin cancer, prostate cancer, cervical cancer, ovarian cancer, bladder cancer, oral cavity cancer, and combinations thereof. In other embodiments, the method treats one or more inflammatory diseases, conditions, and illnesses, including, but not limited to, autoinflammatory disease (AID), cryopyrin-associated periodic syndrome (CAPS), systemic lupus erythematosus (SLE), inflammatory bowel disease (IBD) and combinations thereof. In some embodiments, the method treats one or more cardiac diseases, conditions, and illnesses, including, but not limited to, myocardial infarction, heart failure (e.g., refractory or acute decompensated heart failure), arrythmias, pericarditis (e.g., refractory idiopathic pericarditis), myocarditis, sepsis-induced cardiomyopathy, atherosclerosis, coronary artery disease and combinations thereof. In some embodiments, the method treats one or more neurological diseases, conditions, and illnesses, including, but not limited to, acute thrombotic stroke, epilepsy, multiple sclerosis, Alzheimer's Disease and combinations thereof. In some embodiments, the method treats one or more ophthalmic diseases, conditions, and illnesses, including, but not limited to, uveitis, scleritis, Sjogren asthenia, dry eye and combinations thereof. In some embodiments, the method treats one or more diseases, conditions, and illnesses, including, but not limited to, chronic kidney disease, Type 2 diabetes, gastroesophageal reflux disease (GERD), non-HP-associated peptic ulcer disease, and pulmonary fibrosis.

In some embodiments, the pharmaceutical composition comprising a CRISPR gene-editing system targets TNFRSF1A for the treatment of a joint disease or illness. In some embodiments, the pharmaceutical composition comprising a CRISPR gene-editing system targets TNFRSF1A for the treatment of osteoarthritis. In some embodiments, the pharmaceutical composition comprising a CRISPR gene-editing system targets TNFRSF1A for the treatment of gout.

In some embodiments, the pharmaceutical composition comprising a CRISPR gene-editing system targets TNFRSF1A for the treatment of a musculoskeletal disorder.

In some embodiments, the pharmaceutical composition comprising a CRISPR gene-editing system targets TNFRSF1A for the treatment of a back or spine condition or disorder. In some embodiments, the pharmaceutical composition comprising a CRISPR gene-editing system targets TNFRSF1A for the treatment of low back pain. In some embodiments, the pharmaceutical composition comprising a CRISPR gene-editing system targets TNFRSF1A for the treatment of degenerative disc disorder. In some embodiments, the pharmaceutical composition comprising a CRISPR gene-editing system targets TNFRSF1A for the treatment of internal disc disruption.

### 1. Treatment of Musculoskeletal Disorders by Targeting a Tumor Necrosis Factor Receptor Superfamily Gene

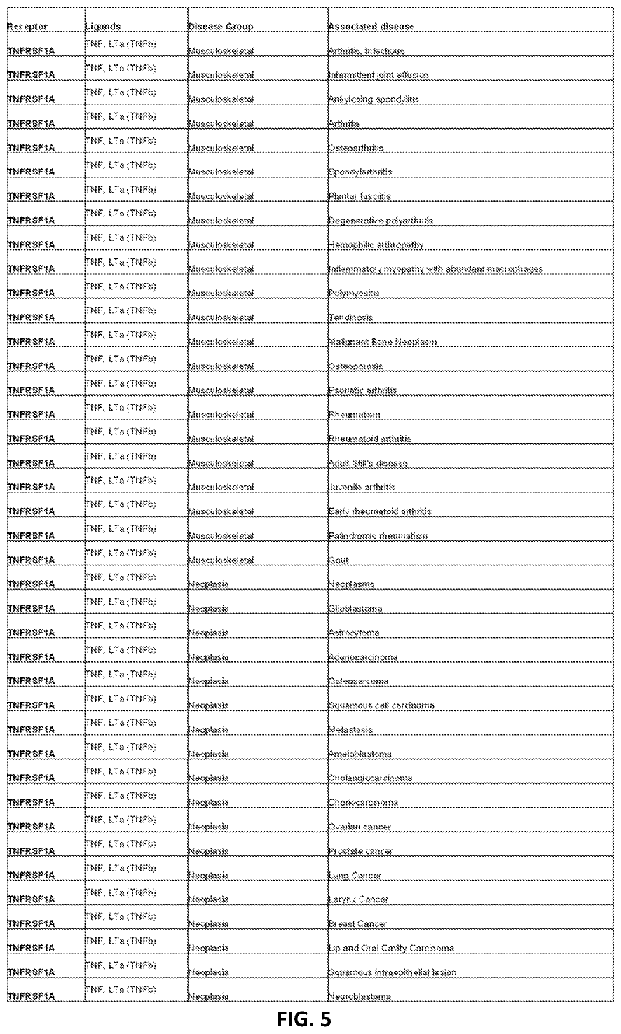

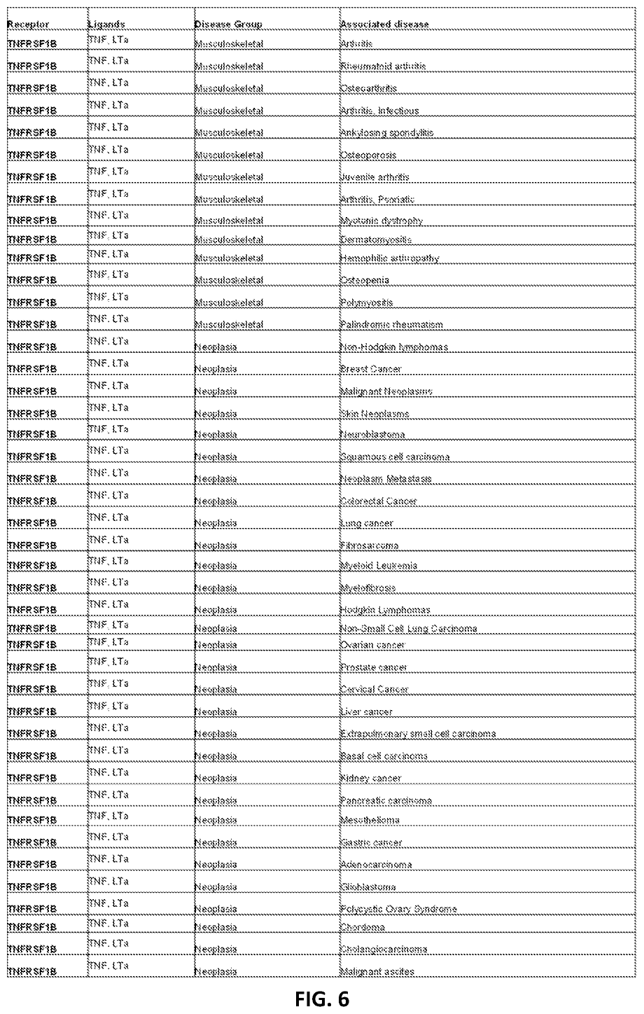

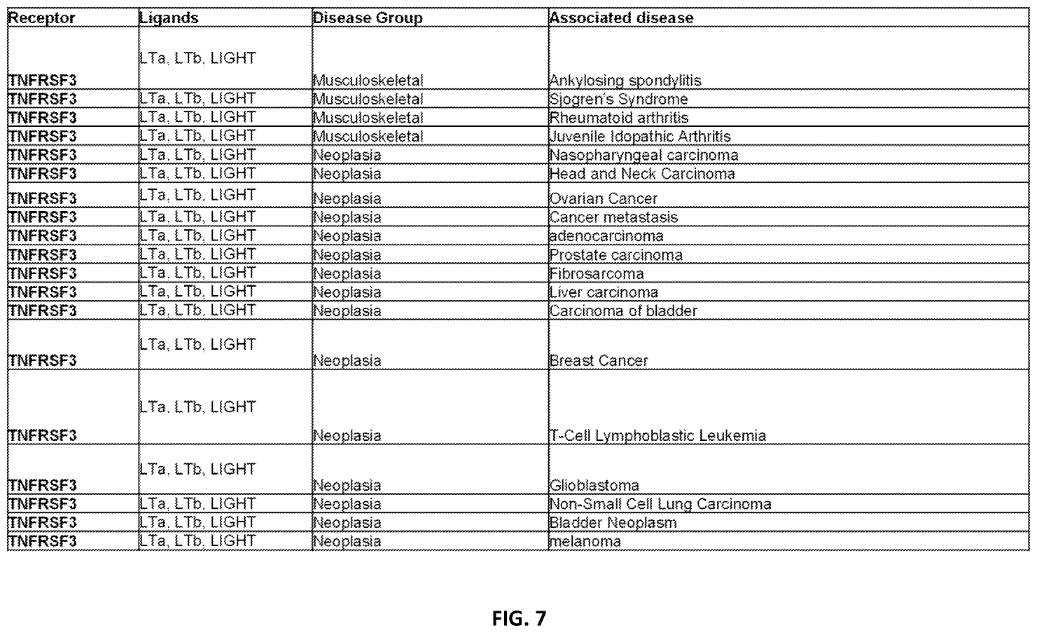

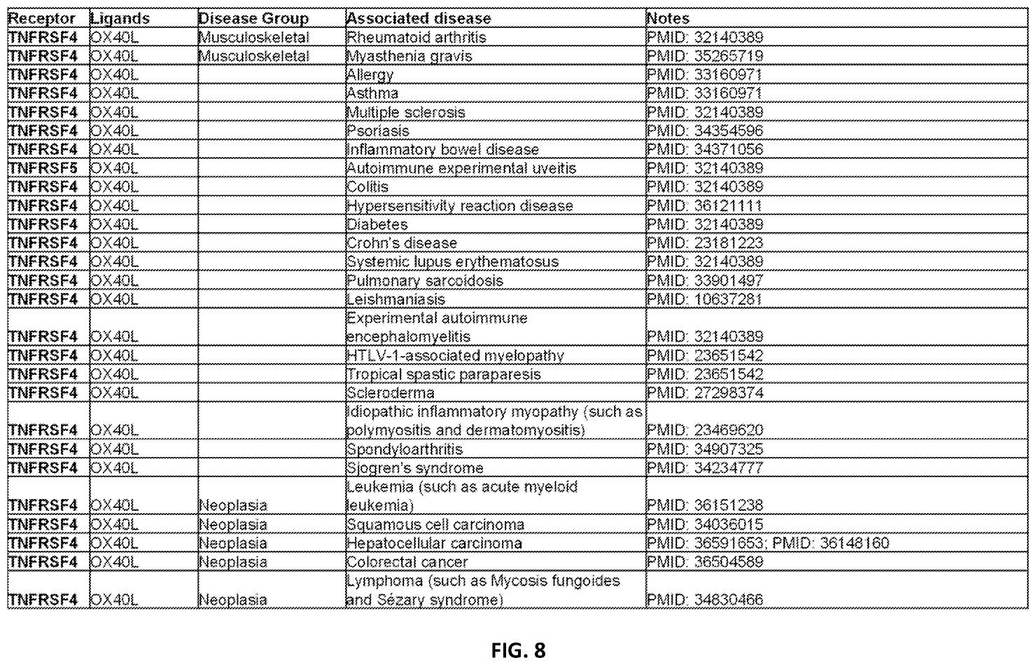

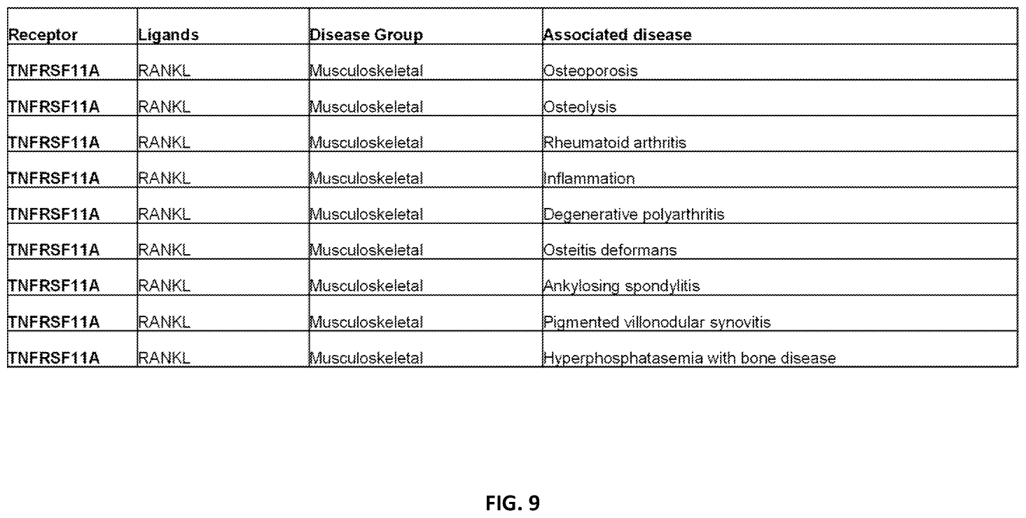

In one aspect, methods and pharmaceutical compositions are provided for treating a musculoskeletal disorder using a CRISPR pharmaceutical composition that targets a Tumor Necrosis Factor Receptor Superfamily gene. In some embodiments, the CRISPR pharmaceutical composition comprises (i) an RNA-guided nuclease or a nucleic acid encoding an RNA-guided nuclease, and (ii) at least one guide RNA (gRNA), or a nucleic acid encoding at least one gRNA, targeting the Tumor Necrosis Factor Receptor Superfamily gene, where the gRNA specifically binds a target sequence that is adjacent to a protospacer adjacent motif (PAM) sequence for the RNA-guided nuclease. In some embodiments, the CRISPR pharmaceutical composition comprises (i) an mRNA encoding an RNA-guided nuclease, and (ii) at least one gRNA targeting the Tumor Necrosis Factor Receptor Superfamily gene. In some embodiments, the mRNA encoding the RNA-guided nuclease and the at least one gRNA are packaged in a lipid nanoparticle (LNP). In some embodiments, the musculoskeletal disorder is Arthritis, Infectious Intermittent joint effusion, Ankylosing spondylitis, Arthritis, Osteoarthritis, Spondylarthrites, Plantar fasciitis, Degenerative polyarthritis, Hemophilic arthropathy, Inflammatory myopathy with abundant macrophages, Polymyositis, Tendinosis, Malignant Bone Neoplasm, Osteoporosis, Psoriatic arthritis, Rheumatism, Rheumatoid arthritis, Adult Still's disease, Juvenile arthritis, Early rheumatoid arthritis, Palindromic rheumatism, Gout, Infectious Arthritis, Myotonic dystrophy, Dermatomyositis, Hemophilic arthropathy, Osteopenia, Sjogren's syndrome, Juvenile Idiopathic Arthritis, Myasthenia gravis, Osteolysis, Inflammation, Degenerative polyarthritis, Osteitis deformans, Pigmented villonodular synovitis, or Hyperphosphatasemia with bone disease. In some embodiments, a musculoskeletal disorder listed in FIG. 5, 6, 7, 8, or 9 is treated with a CRISPR pharmaceutical composition targeting a gene listed as associated with the disorder in FIG. 5, 6, 7, 8, or 9. For example, in some embodiments, Rheumatoid Arthritis is treated with a CRISPR pharmaceutical composition targeting a TNFRSF1A, TNFRSF1B, TNFRSF3, TNFRSF4, or TNFRSF11A gene.

In some embodiments, the tumor necrosis factor receptor superfamily gene is an TNFRSF1A gene. In some embodiments, the TNFRSF1A gene is a human TNFRSF1A gene and the guide RNA comprises a crRNA sequence selected from SEQ ID NOs: 2179-2383. In some embodiments, editing of the TNFRSF1A gene results in a knockout of the TNFRSF1A gene. In some such embodiments, the TNFRSF1A gene is a human TNFRSF1A gene and the guide RNA comprises a crRNA sequence selected from SEQ ID NOs: 2179-2247. In some embodiments, editing of the TNFRSF1A gene results in a gene that expresses a membrane-bound receptor decoy protein. In some such embodiments, the TNFRSF1A gene is a human TNFRSF1A gene and the guide RNA comprises a crRNA sequence selected from SEQ ID NOs: 2248-2369. In some embodiments, editing of the TNFRSF1A gene results in a gene that expresses a soluble receptor decoy protein. In some such embodiments, the TNFRSF1A gene is a human TNFRSF1A gene and the guide RNA comprises a crRNA sequence selected from SEQ ID NOs: 2370-2383.

### 2. Treatment of Neoplasia by Targeting a Tumor Necrosis Factor Receptor Superfamily Gene

In one aspect, methods and pharmaceutical compositions are provided for treating a neoplasia using a CRISPR pharmaceutical composition that targets a Tumor Necrosis Factor Receptor Superfamily gene. In some embodiments, the CRISPR pharmaceutical composition comprises (i) an RNA-guided nuclease or a nucleic acid encoding an RNA-guided nuclease, and (ii) at least one guide RNA (gRNA), or a nucleic acid encoding at least one gRNA, targeting the Tumor Necrosis Factor Receptor Superfamily gene, where the gRNA specifically binds a target sequence that is adjacent to a protospacer adjacent motif (PAM) sequence for the RNA-guided nuclease. In some embodiments, the CRISPR pharmaceutical composition comprises (i) an mRNA encoding an RNA-guided nuclease, and (ii) at least one gRNA targeting the Tumor Necrosis Factor Receptor Superfamily gene. In some embodiments, the mRNA encoding the RNA-guided nuclease and the at least one gRNA are packaged in a lipid nanoparticle (LNP). In some embodiments, the neoplasia is Neoplasms, Glioblastoma, Astrocytoma, Adenocarcinoma, Osteosarcoma, Squamous cell carcinoma, Metastasis, Ameloblastoma, Cholangiocarcinoma, Choriocarcinoma, Ovarian cancer, Prostate cancer, Lung Cancer, Larynx Cancer, Breast Cancer, Lip and Oral Cavity Carcinoma, Squamous intraepithelial lesion, Neuroblastoma, Non-Hodgkin lymphomas, Malignant Neoplasms, Skin Neoplasms, Neuroblastoma, Neoplasm Metastasis, Colorectal Cancer, Fibrosarcoma, Myeloid Leukemia, Myelofibrosis, Hodgkin Lymphomas, Non-Small Cell Lung Carcinoma, Cervical Cancer, Liver cancer, Extrapulmonary small cell carcinoma, Basal cell carcinoma, Kidney cancer, Pancreatic carcinoma, Mesothelioma, Gastric cancer, Polycystic Ovary Syndrome, Chordoma, Cholangiocarcinoma, Malignant ascites, Nasopharyngeal carcinoma, Head and Neck Carcinoma, Cancer metastasis, Prostate carcinoma, Fibrosarcoma, Liver carcinoma, Carcinoma of bladder, T-Cell Lymphoblastic Leukemia, Bladder Neoplasm, melanoma, Leukemia (such as acute myeloid leukemia), Hepatocellular carcinoma, Colorectal cancer, or Lymphoma (such as Mycosis fungoides and Sézary syndrome). In some embodiments, a neoplasia listed in FIG. 5, 6, 7, 8, or 9 is treated with a CRISPR pharmaceutical composition targeting a gene listed as associated with the disorder in FIG. 5, 6, 7, 8, or 9. For example, in some embodiments, Glioblastoma is treated with a CRISPR pharmaceutical composition targeting a TNFRSF1A, TNFRSF1B, TNFRSF3, TNFRSF4, or TNFRSF11A gene.

In some embodiments, the tumor necrosis factor receptor superfamily gene is an TNFRSF1A gene. In some embodiments, the TNFRSF1A gene is a human TNFRSF1A gene and the guide RNA comprises a crRNA sequence selected from SEQ ID NOs: 2179-2383. In some embodiments, editing of the TNFRSF1A gene results in a knockout of the TNFRSF1A gene. In some such embodiments, the TNFRSF1A gene is a human TNFRSF1A gene and the guide RNA comprises a crRNA sequence selected from SEQ ID NOs: 2179-2247. In some embodiments, editing of the TNFRSF1A gene results in a gene that expresses a membrane-bound receptor decoy protein. In some such embodiments, the TNFRSF1A gene is a human TNFRSF1A gene and the guide RNA comprises a crRNA sequence selected from SEQ ID NOs: 2248-2369. In some embodiments, editing of the TNFRSF1A gene results in a gene that expresses a soluble receptor decoy protein. In some such embodiments, the TNFRSF1A gene is a human TNFRSF1A gene and the guide RNA comprises a crRNA sequence selected from SEQ ID NOs: 2370-2383.

### 3. Treatment of Autoimmune or Inflammatory Disorders by Targeting a Tumor Necrosis Factor Receptor Superfamily Gene

In one aspect, methods and pharmaceutical compositions are provided for treating an autoimmune or inflammatory disorder using a CRISPR pharmaceutical composition that targets a Tumor Necrosis Factor Receptor Superfamily gene. In some embodiments, the CRISPR pharmaceutical composition comprises (i) an RNA-guided nuclease or a nucleic acid encoding an RNA-guided nuclease, and (ii) at least one guide RNA (gRNA), or a nucleic acid encoding at least one gRNA, targeting the Tumor Necrosis Factor Receptor Superfamily gene, where the gRNA specifically binds a target sequence that is adjacent to a protospacer adjacent motif (PAM) sequence for the RNA-guided nuclease. In some embodiments, the CRISPR pharmaceutical composition comprises (i) an mRNA encoding an RNA-guided nuclease, and (ii) at least one gRNA targeting the Tumor Necrosis Factor Receptor Superfamily gene. In some embodiments, the mRNA encoding the RNA-guided nuclease and the at least one gRNA are packaged in a lipid nanoparticle (LNP). In some embodiments, the autoimmune or inflammatory disorder is Allergy, Asthma, Multiple sclerosis, Psoriasis, Inflammatory bowel disease, Autoimmune experimental uveitis, Colitis, Hypersensitivity reaction disease, Diabetes, Crohn's disease, Systemic lupus erythematosus, Pulmonary sarcoidosis, Leishmaniasis, Experimental autoimmune encephalomyelitis, HTLV-1-associated myelopathy, Tropical spastic paraparesis, Scleroderma, or Idiopathic inflammatory myopathy (such as polymyositis and dermatomyositis). In some embodiments, an autoimmune or inflammatory disorder listed in FIG. 5, 6, 7, 8, or 9 is treated with a CRISPR pharmaceutical composition targeting a gene listed as associated with the disorder in FIG. 5, 6, 7, 8, or 9. For example, in some embodiments, allergies are treated with a CRISPR pharmaceutical composition targeting a TNFRSF1A, TNFRSF1B, TNFRSF3, TNFRSF4, or TNFRSF11A gene.

In some embodiments, the tumor necrosis factor receptor superfamily gene is an TNFRSF1A gene. In some embodiments, the TNFRSF1A gene is a human TNFRSF1A gene and the guide RNA comprises a crRNA sequence selected from SEQ ID NOs: 2179-2383. In some embodiments, editing of the TNFRSF1A gene results in a knockout of the TNFRSF1A gene. In some such embodiments, the TNFRSF1A gene is a human TNFRSF1A gene and the guide RNA comprises a crRNA sequence selected from SEQ ID NOs: 2179-2247. In some embodiments, editing of the TNFRSF1A gene results in a gene that expresses a membrane-bound receptor decoy protein. In some such embodiments, the TNFRSF1A gene is a human TNFRSF1A gene and the guide RNA comprises a crRNA sequence selected from SEQ ID NOs: 2248-2369. In some embodiments, editing of the TNFRSF1A gene results in a gene that expresses a soluble receptor decoy protein. In some such embodiments, the TNFRSF1A gene is a human TNFRSF1A gene and the guide RNA comprises a crRNA sequence selected from SEQ ID NOs: 2370-2383.

### H. TNFRSF1B

In some embodiments, the pharmaceutical composition comprising a CRISPR gene-editing system targets TNFRSF1B. In some embodiments, the CRISPR gene-editing system targeting TNFRSF1B is delivered to a mammalian cell via viral vector. In some embodiments, the CRISPR gene-editing system targeting TNFRSF1B is delivered to a mammalian cell via an AAV vector. In some embodiments, the CRISPR gene-editing system targeting TNFRSF1B is delivered to a mammalian cell via a lentiviral vector. In some embodiments, the CRISPR gene-editing system targeting TNFRSF1B is delivered to a mammalian cell via a lipid nanoparticle. In some embodiments, the CRISPR gene-editing system targeting TNFRSF1B is delivered to a mammalian cell via a virus-like particle. In some embodiments, the CRISPR gene-editing system targeting TNFRSF1B is delivered to a mammalian cell via a liposome. In some embodiments, the CRISPR gene-editing system targeting TNFRSF1B is delivered to a mammalian cell via a lipid nanocrystal.

In various embodiments, the pharmaceutical composition comprising a CRISPR gene-editing system that targets the TNFRSF1B gene is used in a method of treating a mammal in need thereof. In some embodiments, the method treats one or more joint disease or illness or one or more back or spine conditions or disorders. In some embodiments, the method treats one or more musculoskeletal diseases, conditions, and illnesses, including, but not limited to, rheumatoid arthritis, gout, osteoarthritis, osteoporosis, intervertebral disc disease (IVDD), psoriatic arthritis, arthritis, polymyositis, proliferative synovitis, bone neoplasms, sarcoid myopathy, cortex bone disorders, idiopathic scoliosis, tendinopathy, myofibrillar myopathy, enthesis-related arthritis, ankylosing spondylitis, degenerative polyarthritis, arthropathy, osteitis deformans, prolapsed lumbar disc, polymyositis ossificans, idiopathic polymyositis, Luft Disease, adult-onset Still's Disease, osteoarthritis deformans, Bachet's Disease, and combinations thereof. In other embodiments, use of the system treats one or more neoplastic diseases, conditions, and illnesses, including, but not limited to, osteosarcoma, multiple myeloma, lymphoma (e.g., B-cell or cutaneous T-cell lymphoma), leukemia, thyroid carcinoma, glioma, renal cell carcinoma, chondrosarcoma, glioblastoma, melanoma, neuroblastoma, polycystic ovary syndrome, Kaposi sarcoma, squamous cell carcinoma, leiomyosarcoma, malignant peripheral nerve sheath tumor, Ewing's sarcoma, esophageal neoplasms, colon cancer, lung cancer, breast cancer, pancreatic cancer, stomach cancer, epithelial ovarian cancer, cholectoral cancer, liver cancer, skin cancer, prostate cancer, cervical cancer, ovarian cancer, bladder cancer, oral cavity cancer, and combinations thereof. In other embodiments, the method treats one or more inflammatory diseases, conditions, and illnesses, including, but not limited to, autoinflammatory disease (AID), cryopyrin-associated periodic syndrome (CAPS), systemic lupus erythematosus (SLE), inflammatory bowel disease (IBD) and combinations thereof. In some embodiments, the method treats one or more cardiac diseases, conditions, and illnesses, including, but not limited to, myocardial infarction, heart failure (e.g., refractory or acute decompensated heart failure), arrythmias, pericarditis (e.g., refractory idiopathic pericarditis), myocarditis, sepsis-induced cardiomyopathy, atherosclerosis, coronary artery disease and combinations thereof. In some embodiments, the method treats one or more neurological diseases, conditions, and illnesses, including, but not limited to, acute thrombotic stroke, epilepsy, multiple sclerosis, Alzheimer's Disease and combinations thereof. In some embodiments, the method treats one or more ophthalmic diseases, conditions, and illnesses, including, but not limited to, uveitis, scleritis, Sjogren asthenia, dry eye and combinations thereof. In some embodiments, the method treats one or more diseases, conditions, and illnesses, including, but not limited to, chronic kidney disease, Type 2 diabetes, gastroesophageal reflux disease (GERD), non-HP-associated peptic ulcer disease, and pulmonary fibrosis.

In some embodiments, the pharmaceutical composition comprising a CRISPR gene-editing system targets TNFRSF1B for the treatment of a joint disease or illness. In some embodiments, the pharmaceutical composition comprising a CRISPR gene-editing system targets TNFRSF1B for the treatment of osteoarthritis. In some embodiments, the pharmaceutical composition comprising a CRISPR gene-editing system targets TNFRSF1B for the treatment of gout.

In some embodiments, the pharmaceutical composition comprising a CRISPR gene-editing system targets TNFRSF1B for the treatment of a musculoskeletal disorder.

In some embodiments, the pharmaceutical composition comprising a CRISPR gene-editing system targets TNFRSF1B for the treatment of a back or spine condition or disorder. In some embodiments, the pharmaceutical composition comprising a CRISPR gene-editing system targets TNFRSF1B for the treatment of low back pain. In some embodiments, the pharmaceutical composition comprising a CRISPR gene-editing system targets TNFRSF1B for the treatment of degenerative disc disorder. In some embodiments, the pharmaceutical composition comprising a CRISPR gene-editing system targets TNFRSF1B for the treatment of internal disc disruption.

### 1. Treatment of Musculoskeletal Disorders by Targeting a Tumor Necrosis Factor Receptor Superfamily Gene

In one aspect, methods and pharmaceutical compositions are provided for treating a musculoskeletal disorder using a CRISPR pharmaceutical composition that targets a Tumor Necrosis Factor Receptor Superfamily gene. In some embodiments, the CRISPR pharmaceutical composition comprises (i) an RNA-guided nuclease or a nucleic acid encoding an RNA-guided nuclease, and (ii) at least one guide RNA (gRNA), or a nucleic acid encoding at least one gRNA, targeting the Tumor Necrosis Factor Receptor Superfamily gene, where the gRNA specifically binds a target sequence that is adjacent to a protospacer adjacent motif (PAM) sequence for the RNA-guided nuclease. In some embodiments, the CRISPR pharmaceutical composition comprises (i) an mRNA encoding an RNA-guided nuclease, and (ii) at least one gRNA targeting the Tumor Necrosis Factor Receptor Superfamily gene. In some embodiments, the mRNA encoding the RNA-guided nuclease and the at least one gRNA are packaged in a lipid nanoparticle (LNP). In some embodiments, the musculoskeletal disorder is Arthritis, Infectious Intermittent joint effusion, Ankylosing spondylitis, Arthritis, Osteoarthritis, Spondylarthrites, Plantar fasciitis, Degenerative polyarthritis, Hemophilic arthropathy, Inflammatory myopathy with abundant macrophages, Polymyositis, Tendinosis, Malignant Bone Neoplasm, Osteoporosis, Psoriatic arthritis, Rheumatism, Rheumatoid arthritis, Adult Still's disease, Juvenile arthritis, Early rheumatoid arthritis, Palindromic rheumatism, Gout, Infectious Arthritis, Myotonic dystrophy, Dermatomyositis, Hemophilic arthropathy, Osteopenia, Sjogren's syndrome, Juvenile Idiopathic Arthritis, Myasthenia gravis, Osteolysis, Inflammation, Degenerative polyarthritis, Osteitis deformans, Pigmented villonodular synovitis, or Hyperphosphatasemia with bone disease. In some embodiments, a musculoskeletal disorder listed in FIG. 5, 6, 7, 8, or 9 is treated with a CRISPR pharmaceutical composition targeting a gene listed as associated with the disorder in FIG. 5, 6, 7, 8, or 9. For example, in some embodiments, Rheumatoid Arthritis is treated with a CRISPR pharmaceutical composition targeting a TNFRSF1A, TNFRSF1B, TNFRSF3, TNFRSF4, or TNFRSF11A gene.

In some embodiments, the tumor necrosis factor receptor superfamily gene is an TNFRSF1B gene. In some embodiments, the TNFRSF1B gene is a human TNFRSF1B gene and the guide RNA comprises a crRNA sequence selected from SEQ ID NOs: 2396-2642. In some embodiments, editing of the TNFRSF1B gene results in a knockout of the TNFRSF1B gene. In some such embodiments, the TNFRSF1B gene is a human TNFRSF1B gene and the guide RNA comprises a crRNA sequence selected from SEQ ID NOs: 2396-2482. In some embodiments, editing of the TNFRSF1B gene results in a gene that expresses a membrane-bound receptor decoy protein. In some such embodiments, the TNFRSF1B gene is a human TNFRSF1B gene and the guide RNA comprises a crRNA sequence selected from SEQ ID NOs: 2483-2574. In some embodiments, editing of the TNFRSF1B gene results in a gene that expresses a soluble receptor decoy protein. In some such embodiments, the TNFRSF1B gene is a human TNFRSF1B gene and the guide RNA comprises a crRNA sequence selected from SEQ ID NOs: 2575-2622.

### 2. Treatment of Neoplasia by Targeting a Tumor Necrosis Factor Receptor Superfamily Gene

In one aspect, methods and pharmaceutical compositions are provided for treating a neoplasia using a CRISPR pharmaceutical composition that targets a Tumor Necrosis Factor Receptor Superfamily gene. In some embodiments, the CRISPR pharmaceutical composition comprises (i) an RNA-guided nuclease or a nucleic acid encoding an RNA-guided nuclease, and (ii) at least one guide RNA (gRNA), or a nucleic acid encoding at least one gRNA, targeting the Tumor Necrosis Factor Receptor Superfamily gene, where the gRNA specifically binds a target sequence that is adjacent to a protospacer adjacent motif (PAM) sequence for the RNA-guided nuclease. In some embodiments, the CRISPR pharmaceutical composition comprises (i) an mRNA encoding an RNA-guided nuclease, and (ii) at least one gRNA targeting the Tumor Necrosis Factor Receptor Superfamily gene. In some embodiments, the mRNA encoding the RNA-guided nuclease and the at least one gRNA are packaged in a lipid nanoparticle (LNP). In some embodiments, the neoplasia is Neoplasms, Glioblastoma, Astrocytoma, Adenocarcinoma, Osteosarcoma, Squamous cell carcinoma, Metastasis, Ameloblastoma, Cholangiocarcinoma, Choriocarcinoma, Ovarian cancer, Prostate cancer, Lung Cancer, Larynx Cancer, Breast Cancer, Lip and Oral Cavity Carcinoma, Squamous intraepithelial lesion, Neuroblastoma, Non-Hodgkin lymphomas, Malignant Neoplasms, Skin Neoplasms, Neuroblastoma, Neoplasm Metastasis, Colorectal Cancer, Fibrosarcoma, Myeloid Leukemia, Myelofibrosis, Hodgkin Lymphomas, Non-Small Cell Lung Carcinoma, Cervical Cancer, Liver cancer, Extrapulmonary small cell carcinoma, Basal cell carcinoma, Kidney cancer, Pancreatic carcinoma, Mesothelioma, Gastric cancer, Polycystic Ovary Syndrome, Chordoma, Cholangiocarcinoma, Malignant ascites, Nasopharyngeal carcinoma, Head and Neck Carcinoma, Cancer metastasis, Prostate carcinoma, Fibrosarcoma, Liver carcinoma, Carcinoma of bladder, T-Cell Lymphoblastic Leukemia, Bladder Neoplasm, melanoma, Leukemia (such as acute myeloid leukemia), Hepatocellular carcinoma, Colorectal cancer, or Lymphoma (such as Mycosis fungoides and Sézary syndrome). In some embodiments, a neoplasia listed in FIG. 5, 6, 7, 8, or 9 is treated with a CRISPR pharmaceutical composition targeting a gene listed as associated with the disorder in FIG. 5, 6, 7, 8, or 9. For example, in some embodiments, Glioblastoma is treated with a CRISPR pharmaceutical composition targeting a TNFRSF1A, TNFRSF1B, TNFRSF3, TNFRSF4, or TNFRSF11A gene.

In some embodiments, the tumor necrosis factor receptor superfamily gene is an TNFRSF1B gene. In some embodiments, the TNFRSF1B gene is a human TNFRSF1B gene and the guide RNA comprises a crRNA sequence selected from SEQ ID NOs: 2396-2642. In some embodiments, editing of the TNFRSF1B gene results in a knockout of the TNFRSF1B gene. In some such embodiments, the TNFRSF1B gene is a human TNFRSF1B gene and the guide RNA comprises a crRNA sequence selected from SEQ ID NOs: 2396-2482. In some embodiments, editing of the TNFRSF1B gene results in a gene that expresses a membrane-bound receptor decoy protein. In some such embodiments, the TNFRSF1B gene is a human TNFRSF1B gene and the guide RNA comprises a crRNA sequence selected from SEQ ID NOs: 2483-2574. In some embodiments, editing of the TNFRSF1B gene results in a gene that expresses a soluble receptor decoy protein. In some such embodiments, the TNFRSF1B gene is a human TNFRSF1B gene and the guide RNA comprises a crRNA sequence selected from SEQ ID NOs: 2575-2622.

### 3. Treatment of Autoimmune or Inflammatory Disorders by Targeting a Tumor Necrosis Factor Receptor Superfamily Gene

In one aspect, methods and pharmaceutical compositions are provided for treating an autoimmune or inflammatory disorder using a CRISPR pharmaceutical composition that targets a Tumor Necrosis Factor Receptor Superfamily gene. In some embodiments, the CRISPR pharmaceutical composition comprises (i) an RNA-guided nuclease or a nucleic acid encoding an RNA-guided nuclease, and (ii) at least one guide RNA (gRNA), or a nucleic acid encoding at least one gRNA, targeting the Tumor Necrosis Factor Receptor Superfamily gene, where the gRNA specifically binds a target sequence that is adjacent to a protospacer adjacent motif (PAM) sequence for the RNA-guided nuclease. In some embodiments, the CRISPR pharmaceutical composition comprises (i) an mRNA encoding an RNA-guided nuclease, and (ii) at least one gRNA targeting the Tumor Necrosis Factor Receptor Superfamily gene. In some embodiments, the mRNA encoding the RNA-guided nuclease and the at least one gRNA are packaged in a lipid nanoparticle (LNP). In some embodiments, the autoimmune or inflammatory disorder is Allergy, Asthma, Multiple sclerosis, Psoriasis, Inflammatory bowel disease, Autoimmune experimental uveitis, Colitis, Hypersensitivity reaction disease, Diabetes, Crohn's disease, Systemic lupus erythematosus, Pulmonary sarcoidosis, Leishmaniasis, Experimental autoimmune encephalomyelitis, HTLV-1-associated myelopathy, Tropical spastic paraparesis, Scleroderma, or Idiopathic inflammatory myopathy (such as polymyositis and dermatomyositis). In some embodiments, an autoimmune or inflammatory disorder listed in FIG. 5, 6, 7, 8, or 9 is treated with a CRISPR pharmaceutical composition targeting a gene listed as associated with the disorder in FIG. 5, 6, 7, 8, or 9. For example, in some embodiments, allergies are treated with a CRISPR pharmaceutical composition targeting a TNFRSF1A, TNFRSF1B, TNFRSF3, TNFRSF4, or TNFRSF11A gene.

In some embodiments, the tumor necrosis factor receptor superfamily gene is an TNFRSF1B gene. In some embodiments, the TNFRSF1B gene is a human TNFRSF1B gene and the guide RNA comprises a crRNA sequence selected from SEQ ID NOs: 2396-2642. In some embodiments, editing of the TNFRSF1B gene results in a knockout of the TNFRSF1B gene. In some such embodiments, the TNFRSF1B gene is a human TNFRSF1B gene and the guide RNA comprises a crRNA sequence selected from SEQ ID NOs: 2396-2482. In some embodiments, editing of the TNFRSF1B gene results in a gene that expresses a membrane-bound receptor decoy protein. In some such embodiments, the TNFRSF1B gene is a human TNFRSF1B gene and the guide RNA comprises a crRNA sequence selected from SEQ ID NOs: 2483-2574. In some embodiments, editing of the TNFRSF1B gene results in a gene that expresses a soluble receptor decoy protein. In some such embodiments, the TNFRSF1B gene is a human TNFRSF1B gene and the guide RNA comprises a crRNA sequence selected from SEQ ID NOs: 2575-2622.

### I. TNFRSF3

In some embodiments, the pharmaceutical composition comprising a CRISPR gene-editing system targets TNFRSF3. In some embodiments, the CRISPR gene-editing system targeting TNFRSF3 is delivered to a mammalian cell via viral vector. In some embodiments, the CRISPR gene-editing system targeting TNFRSF3 is delivered to a mammalian cell via an AAV vector. In some embodiments, the CRISPR gene-editing system targeting TNFRSF3 is delivered to a mammalian cell via a lentiviral vector. In some embodiments, the CRISPR gene-editing system targeting TNFRSF3 is delivered to a mammalian cell via a lipid nanoparticle. In some embodiments, the CRISPR gene-editing system targeting TNFRSF3 is delivered to a mammalian cell via a virus-like particle. In some embodiments, the CRISPR gene-editing system targeting TNFRSF3 is delivered to a mammalian cell via a liposome. In some embodiments, the CRISPR gene-editing system targeting TNFRSF3 is delivered to a mammalian cell via a lipid nanocrystal.

In various embodiments, the pharmaceutical composition comprising a CRISPR gene-editing system that targets the TNFRSF3 gene is used in a method of treating a mammal in need thereof. In some embodiments, the method treats one or more joint disease or illness or one or more back or spine conditions or disorders. In some embodiments, the method treats one or more musculoskeletal diseases, conditions, and illnesses, including, but not limited to, rheumatoid arthritis, gout, osteoarthritis, osteoporosis, intervertebral disc disease (IVDD), psoriatic arthritis, arthritis, polymyositis, proliferative synovitis, bone neoplasms, sarcoid myopathy, cortex bone disorders, idiopathic scoliosis, tendinopathy, myofibrillar myopathy, enthesis-related arthritis, ankylosing spondylitis, degenerative polyarthritis, arthropathy, osteitis deformans, prolapsed lumbar disc, polymyositis ossificans, idiopathic polymyositis, Luft Disease, adult-onset Still's Disease, osteoarthritis deformans, Bachet's Disease, and combinations thereof. In other embodiments, use of the system treats one or more neoplastic diseases, conditions, and illnesses, including, but not limited to, osteosarcoma, multiple myeloma, lymphoma (e.g., B-cell or cutaneous T-cell lymphoma), leukemia, thyroid carcinoma, glioma, renal cell carcinoma, chondrosarcoma, glioblastoma, melanoma, neuroblastoma, polycystic ovary syndrome, Kaposi sarcoma, squamous cell carcinoma, leiomyosarcoma, malignant peripheral nerve sheath tumor, Ewing's sarcoma, esophageal neoplasms, colon cancer, lung cancer, breast cancer, pancreatic cancer, stomach cancer, epithelial ovarian cancer, cholectoral cancer, liver cancer, skin cancer, prostate cancer, cervical cancer, ovarian cancer, bladder cancer, oral cavity cancer, and combinations thereof. In other embodiments, the method treats one or more inflammatory diseases, conditions, and illnesses, including, but not limited to, autoinflammatory disease (AID), cryopyrin-associated periodic syndrome (CAPS), systemic lupus erythematosus (SLE), inflammatory bowel disease (IBD) and combinations thereof. In some embodiments, the method treats one or more cardiac diseases, conditions, and illnesses, including, but not limited to, myocardial infarction, heart failure (e.g., refractory or acute decompensated heart failure), arrythmias, pericarditis (e.g., refractory idiopathic pericarditis), myocarditis, sepsis-induced cardiomyopathy, atherosclerosis, coronary artery disease and combinations thereof. In some embodiments, the method treats one or more neurological diseases, conditions, and illnesses, including, but not limited to, acute thrombotic stroke, epilepsy, multiple sclerosis, Alzheimer's Disease and combinations thereof. In some embodiments, the method treats one or more ophthalmic diseases, conditions, and illnesses, including, but not limited to, uveitis, scleritis, Sjogren asthenia, dry eye and combinations thereof. In some embodiments, the method treats one or more diseases, conditions, and illnesses, including, but not limited to, chronic kidney disease, Type 2 diabetes, gastroesophageal reflux disease (GERD), non-HP-associated peptic ulcer disease, and pulmonary fibrosis.

In some embodiments, the pharmaceutical composition comprising a CRISPR gene-editing system targets TNFRSF3 for the treatment of a joint disease or illness. In some embodiments, the pharmaceutical composition comprising a CRISPR gene-editing system targets TNFRSF3 for the treatment of osteoarthritis. In some embodiments, the pharmaceutical composition comprising a CRISPR gene-editing system targets TNFRSF3 for the treatment of gout.

In some embodiments, the pharmaceutical composition comprising a CRISPR gene-editing system targets TNFRSF3 for the treatment of a musculoskeletal disorder.

In some embodiments, the pharmaceutical composition comprising a CRISPR gene-editing system targets TNFRSF3 for the treatment of a back or spine condition or disorder. In some embodiments, the pharmaceutical composition comprising a CRISPR gene-editing system targets TNFRSF3 for the treatment of low back pain. In some embodiments, the pharmaceutical composition comprising a CRISPR gene-editing system targets TNFRSF3 for the treatment of degenerative disc disorder. In some embodiments, the pharmaceutical composition comprising a CRISPR gene-editing system targets TNFRSF3 for the treatment of internal disc disruption.

### 1. Treatment of Musculoskeletal Disorders by Targeting a Tumor Necrosis Factor Receptor Superfamily Gene

In one aspect, methods and pharmaceutical compositions are provided for treating a musculoskeletal disorder using a CRISPR pharmaceutical composition that targets a Tumor Necrosis Factor Receptor Superfamily gene. In some embodiments, the CRISPR pharmaceutical composition comprises (i) an RNA-guided nuclease or a nucleic acid encoding an RNA-guided nuclease, and (ii) at least one guide RNA (gRNA), or a nucleic acid encoding at least one gRNA, targeting the Tumor Necrosis Factor Receptor Superfamily gene, where the gRNA specifically binds a target sequence that is adjacent to a protospacer adjacent motif (PAM) sequence for the RNA-guided nuclease. In some embodiments, the CRISPR pharmaceutical composition comprises (i) an mRNA encoding an RNA-guided nuclease, and (ii) at least one gRNA targeting the Tumor Necrosis Factor Receptor Superfamily gene. In some embodiments, the mRNA encoding the RNA-guided nuclease and the at least one gRNA are packaged in a lipid nanoparticle (LNP). In some embodiments, the musculoskeletal disorder is Arthritis, Infectious Intermittent joint effusion, Ankylosing spondylitis, Arthritis, Osteoarthritis, Spondylarthrites, Plantar fasciitis, Degenerative polyarthritis, Hemophilic arthropathy, Inflammatory myopathy with abundant macrophages, Polymyositis, Tendinosis, Malignant Bone Neoplasm, Osteoporosis, Psoriatic arthritis, Rheumatism, Rheumatoid arthritis, Adult Still's disease, Juvenile arthritis, Early rheumatoid arthritis, Palindromic rheumatism, Gout, Infectious Arthritis, Myotonic dystrophy, Dermatomyositis, Hemophilic arthropathy, Osteopenia, Sjogren's syndrome, Juvenile Idiopathic Arthritis, Myasthenia gravis, Osteolysis, Inflammation, Degenerative polyarthritis, Osteitis deformans, Pigmented villonodular synovitis, or Hyperphosphatasemia with bone disease. In some embodiments, a musculoskeletal disorder listed in FIG. 5, 6, 7, 8, or 9 is treated with a CRISPR pharmaceutical composition targeting a gene listed as associated with the disorder in FIG. 5, 6, 7, 8, or 9. For example, in some embodiments, Rheumatoid Arthritis is treated with a CRISPR pharmaceutical composition targeting a TNFRSF1A, TNFRSF1B, TNFRSF3, TNFRSF4, or TNFRSF11A gene.

In some embodiments, the tumor necrosis factor receptor superfamily gene is an TNFRSF3 gene. In some embodiments, the TNFRSF3 gene is a human TNFRSF3 gene and the guide RNA comprises a crRNA sequence selected from SEQ ID NOs: 2643-2866. In some embodiments, editing of the TNFRSF3 gene results in a knockout of the TNFRSF3 gene. In some such embodiments, the TNFRSF3 gene is a human TNFRSF3 gene and the guide RNA comprises a crRNA sequence selected from SEQ ID NOs: 2643-2737. In some embodiments, editing of the TNFRSF3 gene results in a gene that expresses a membrane-bound receptor decoy protein. In some such embodiments, the TNFRSF3 gene is a human TNFRSF3 gene and the guide RNA comprises a crRNA sequence selected from SEQ ID NOs: 2738-2846. In some embodiments, editing of the TNFRSF3 gene results in a gene that expresses a soluble receptor decoy protein. In some such embodiments, the TNFRSF3 gene is a human TNFRSF3 gene and the guide RNA comprises a crRNA sequence selected from SEQ ID NOs: 2847-2852.

### 2. Treatment of Neoplasia by Targeting a Tumor Necrosis Factor Receptor Superfamily Gene

In one aspect, methods and pharmaceutical compositions are provided for treating a neoplasia using a CRISPR pharmaceutical composition that targets a Tumor Necrosis Factor Receptor Superfamily gene. In some embodiments, the CRISPR pharmaceutical composition comprises (i) an RNA-guided nuclease or a nucleic acid encoding an RNA-guided nuclease, and (ii) at least one guide RNA (gRNA), or a nucleic acid encoding at least one gRNA, targeting the Tumor Necrosis Factor Receptor Superfamily gene, where the gRNA specifically binds a target sequence that is adjacent to a protospacer adjacent motif (PAM) sequence for the RNA-guided nuclease. In some embodiments, the CRISPR pharmaceutical composition comprises (i) an mRNA encoding an RNA-guided nuclease, and (ii) at least one gRNA targeting the Tumor Necrosis Factor Receptor Superfamily gene. In some embodiments, the mRNA encoding the RNA-guided nuclease and the at least one gRNA are packaged in a lipid nanoparticle (LNP). In some embodiments, the neoplasia is Neoplasms, Glioblastoma, Astrocytoma, Adenocarcinoma, Osteosarcoma, Squamous cell carcinoma, Metastasis, Ameloblastoma, Cholangiocarcinoma, Choriocarcinoma, Ovarian cancer, Prostate cancer, Lung Cancer, Larynx Cancer, Breast Cancer, Lip and Oral Cavity Carcinoma, Squamous intraepithelial lesion, Neuroblastoma, Non-Hodgkin lymphomas, Malignant Neoplasms, Skin Neoplasms, Neuroblastoma, Neoplasm Metastasis, Colorectal Cancer, Fibrosarcoma, Myeloid Leukemia, Myelofibrosis, Hodgkin Lymphomas, Non-Small Cell Lung Carcinoma, Cervical Cancer, Liver cancer, Extrapulmonary small cell carcinoma, Basal cell carcinoma, Kidney cancer, Pancreatic carcinoma, Mesothelioma, Gastric cancer, Polycystic Ovary Syndrome, Chordoma, Cholangiocarcinoma, Malignant ascites, Nasopharyngeal carcinoma, Head and Neck Carcinoma, Cancer metastasis, Prostate carcinoma, Fibrosarcoma, Liver carcinoma, Carcinoma of bladder, T-Cell Lymphoblastic Leukemia, Bladder Neoplasm, melanoma, Leukemia (such as acute myeloid leukemia), Hepatocellular carcinoma, Colorectal cancer, or Lymphoma (such as Mycosis fungoides and Sézary syndrome). In some embodiments, a neoplasia listed in FIG. 5, 6, 7, 8, or 9 is treated with a CRISPR pharmaceutical composition targeting a gene listed as associated with the disorder in FIG. 5, 6, 7, 8, or 9. For example, in some embodiments, Glioblastoma is treated with a CRISPR pharmaceutical composition targeting a TNFRSF1A, TNFRSF1B, TNFRSF3, TNFRSF4, or TNFRSF11A gene.

In some embodiments, the tumor necrosis factor receptor superfamily gene is an TNFRSF3 gene. In some embodiments, the TNFRSF3 gene is a human TNFRSF3 gene and the guide RNA comprises a crRNA sequence selected from SEQ ID NOs: 2643-2866. In some embodiments, editing of the TNFRSF3 gene results in a knockout of the TNFRSF3 gene. In some such embodiments, the TNFRSF3 gene is a human TNFRSF3 gene and the guide RNA comprises a crRNA sequence selected from SEQ ID NOs: 2643-2737. In some embodiments, editing of the TNFRSF3 gene results in a gene that expresses a membrane-bound receptor decoy protein. In some such embodiments, the TNFRSF3 gene is a human TNFRSF3 gene and the guide RNA comprises a crRNA sequence selected from SEQ ID NOs: 2738-2846. In some embodiments, editing of the TNFRSF3 gene results in a gene that expresses a soluble receptor decoy protein. In some such embodiments, the TNFRSF3 gene is a human TNFRSF3 gene and the guide RNA comprises a crRNA sequence selected from SEQ ID NOs: 2847-2852.

### 3. Treatment of Autoimmune or Inflammatory Disorders by Targeting a Tumor Necrosis Factor Receptor Superfamily Gene

In one aspect, methods and pharmaceutical compositions are provided for treating an autoimmune or inflammatory disorder using a CRISPR pharmaceutical composition that targets a Tumor Necrosis Factor Receptor Superfamily gene. In some embodiments, the CRISPR pharmaceutical composition comprises (i) an RNA-guided nuclease or a nucleic acid encoding an RNA-guided nuclease, and (ii) at least one guide RNA (gRNA), or a nucleic acid encoding at least one gRNA, targeting the Tumor Necrosis Factor Receptor Superfamily gene, where the gRNA specifically binds a target sequence that is adjacent to a protospacer adjacent motif (PAM) sequence for the RNA-guided nuclease. In some embodiments, the CRISPR pharmaceutical composition comprises (i) an mRNA encoding an RNA-guided nuclease, and (ii) at least one gRNA targeting the Tumor Necrosis Factor Receptor Superfamily gene. In some embodiments, the mRNA encoding the RNA-guided nuclease and the at least one gRNA are packaged in a lipid nanoparticle (LNP). In some embodiments, the autoimmune or inflammatory disorder is Allergy, Asthma, Multiple sclerosis, Psoriasis, Inflammatory bowel disease, Autoimmune experimental uveitis, Colitis, Hypersensitivity reaction disease, Diabetes, Crohn's disease, Systemic lupus erythematosus, Pulmonary sarcoidosis, Leishmaniasis, Experimental autoimmune encephalomyelitis, HTLV-1-associated myelopathy, Tropical spastic paraparesis, Scleroderma, or Idiopathic inflammatory myopathy (such as polymyositis and dermatomyositis). In some embodiments, an autoimmune or inflammatory disorder listed in FIG. 5, 6, 7, 8, or 9 is treated with a CRISPR pharmaceutical composition targeting a gene listed as associated with the disorder in FIG. 5, 6, 7, 8, or 9. For example, in some embodiments, allergies are treated with a CRISPR pharmaceutical composition targeting a TNFRSF1A, TNFRSF1B, TNFRSF3, TNFRSF4, or TNFRSF11A gene.

In some embodiments, the tumor necrosis factor receptor superfamily gene is an TNFRSF3 gene. In some embodiments, the TNFRSF3 gene is a human TNFRSF3 gene and the guide RNA comprises a crRNA sequence selected from SEQ ID NOs: 2643-2866. In some embodiments, editing of the TNFRSF3 gene results in a knockout of the TNFRSF3 gene. In some such embodiments, the TNFRSF3 gene is a human TNFRSF3 gene and the guide RNA comprises a crRNA sequence selected from SEQ ID NOs: 2643-2737. In some embodiments, editing of the TNFRSF3 gene results in a gene that expresses a membrane-bound receptor decoy protein. In some such embodiments, the TNFRSF3 gene is a human TNFRSF3 gene and the guide RNA comprises a crRNA sequence selected from SEQ ID NOs: 2738-2846. In some embodiments, editing of the TNFRSF3 gene results in a gene that expresses a soluble receptor decoy protein. In some such embodiments, the TNFRSF3 gene is a human TNFRSF3 gene and the guide RNA comprises a crRNA sequence selected from SEQ ID NOs: 2847-2852.

### J. TNFRSF4

In some embodiments, the pharmaceutical composition comprising a CRISPR gene-editing system targets TNFRSF4. In some embodiments, the CRISPR gene-editing system targeting TNFRSF4 is delivered to a mammalian cell via viral vector. In some embodiments, the CRISPR gene-editing system targeting TNFRSF4 is delivered to a mammalian cell via an AAV vector. In some embodiments, the CRISPR gene-editing system targeting TNFRSF4 is delivered to a mammalian cell via a lentiviral vector. In some embodiments, the CRISPR gene-editing system targeting TNFRSF4 is delivered to a mammalian cell via a lipid nanoparticle. In some embodiments, the CRISPR gene-editing system targeting TNFRSF4 is delivered to a mammalian cell via a virus-like particle. In some embodiments, the CRISPR gene-editing system targeting TNFRSF4 is delivered to a mammalian cell via a liposome. In some embodiments, the CRISPR gene-editing system targeting TNFRSF4 is delivered to a mammalian cell via a lipid nanocrystal.

In various embodiments, the pharmaceutical composition comprising a CRISPR gene-editing system that targets the TNFRSF4 gene is used in a method of treating a mammal in need thereof. In some embodiments, the method treats one or more joint disease or illness or one or more back or spine conditions or disorders. In some embodiments, the method treats one or more musculoskeletal diseases, conditions, and illnesses, including, but not limited to, rheumatoid arthritis, gout, osteoarthritis, osteoporosis, intervertebral disc disease (IVDD), psoriatic arthritis, arthritis, polymyositis, proliferative synovitis, bone neoplasms, sarcoid myopathy, cortex bone disorders, idiopathic scoliosis, tendinopathy, myofibrillar myopathy, enthesis-related arthritis, ankylosing spondylitis, degenerative polyarthritis, arthropathy, osteitis deformans, prolapsed lumbar disc, polymyositis ossificans, idiopathic polymyositis, Luft Disease, adult-onset Still's Disease, osteoarthritis deformans, Bachet's Disease, and combinations thereof. In other embodiments, use of the system treats one or more neoplastic diseases, conditions, and illnesses, including, but not limited to, osteosarcoma, multiple myeloma, lymphoma (e.g., B-cell or cutaneous T-cell lymphoma), leukemia, thyroid carcinoma, glioma, renal cell carcinoma, chondrosarcoma, glioblastoma, melanoma, neuroblastoma, polycystic ovary syndrome, Kaposi sarcoma, squamous cell carcinoma, leiomyosarcoma, malignant peripheral nerve sheath tumor, Ewing's sarcoma, esophageal neoplasms, colon cancer, lung cancer, breast cancer, pancreatic cancer, stomach cancer, epithelial ovarian cancer, cholectoral cancer, liver cancer, skin cancer, prostate cancer, cervical cancer, ovarian cancer, bladder cancer, oral cavity cancer, and combinations thereof. In other embodiments, the method treats one or more inflammatory diseases, conditions, and illnesses, including, but not limited to, autoinflammatory disease (AID), cryopyrin-associated periodic syndrome (CAPS), systemic lupus erythematosus (SLE), inflammatory bowel disease (IBD) and combinations thereof. In some embodiments, the method treats one or more cardiac diseases, conditions, and illnesses, including, but not limited to, myocardial infarction, heart failure (e.g., refractory or acute decompensated heart failure), arrythmias, pericarditis (e.g., refractory idiopathic pericarditis), myocarditis, sepsis-induced cardiomyopathy, atherosclerosis, coronary artery disease and combinations thereof. In some embodiments, the method treats one or more neurological diseases, conditions, and illnesses, including, but not limited to, acute thrombotic stroke, epilepsy, multiple sclerosis, Alzheimer's Disease and combinations thereof. In some embodiments, the method treats one or more ophthalmic diseases, conditions, and illnesses, including, but not limited to, uveitis, scleritis, Sjogren asthenia, dry eye and combinations thereof. In some embodiments, the method treats one or more diseases, conditions, and illnesses, including, but not limited to, chronic kidney disease, Type 2 diabetes, gastroesophageal reflux disease (GERD), non-HP-associated peptic ulcer disease, and pulmonary fibrosis.

In some embodiments, the pharmaceutical composition comprising a CRISPR gene-editing system targets TNFRSF4 for the treatment of a joint disease or illness. In some embodiments, the pharmaceutical composition comprising a CRISPR gene-editing system targets TNFRSF4 for the treatment of osteoarthritis. In some embodiments, the pharmaceutical composition comprising a CRISPR gene-editing system targets TNFRSF4 for the treatment of gout.

In some embodiments, the pharmaceutical composition comprising a CRISPR gene-editing system targets TNFRSF4 for the treatment of a musculoskeletal disorder.

In some embodiments, the pharmaceutical composition comprising a CRISPR gene-editing system targets TNFRSF4 for the treatment of a back or spine condition or disorder. In some embodiments, the pharmaceutical composition comprising a CRISPR gene-editing system targets TNFRSF4 for the treatment of low back pain. In some embodiments, the pharmaceutical composition comprising a CRISPR gene-editing system targets TNFRSF4 for the treatment of degenerative disc disorder. In some embodiments, the pharmaceutical composition comprising a CRISPR gene-editing system targets TNFRSF4 for the treatment of internal disc disruption.

### 1. Treatment of Musculoskeletal Disorders by Targeting a Tumor Necrosis Factor Receptor Superfamily Gene

In one aspect, methods and pharmaceutical compositions are provided for treating a musculoskeletal disorder using a CRISPR pharmaceutical composition that targets a Tumor Necrosis Factor Receptor Superfamily gene. In some embodiments, the CRISPR pharmaceutical composition comprises (i) an RNA-guided nuclease or a nucleic acid encoding an RNA-guided nuclease, and (ii) at least one guide RNA (gRNA), or a nucleic acid encoding at least one gRNA, targeting the Tumor Necrosis Factor Receptor Superfamily gene, where the gRNA specifically binds a target sequence that is adjacent to a protospacer adjacent motif (PAM) sequence for the RNA-guided nuclease. In some embodiments, the CRISPR pharmaceutical composition comprises (i) an mRNA encoding an RNA-guided nuclease, and (ii) at least one gRNA targeting the Tumor Necrosis Factor Receptor Superfamily gene. In some embodiments, the mRNA encoding the RNA-guided nuclease and the at least one gRNA are packaged in a lipid nanoparticle (LNP). In some embodiments, the musculoskeletal disorder is Arthritis, Infectious Intermittent joint effusion, Ankylosing spondylitis, Arthritis, Osteoarthritis, Spondylarthrites, Plantar fasciitis, Degenerative polyarthritis, Hemophilic arthropathy, Inflammatory myopathy with abundant macrophages, Polymyositis, Tendinosis, Malignant Bone Neoplasm, Osteoporosis, Psoriatic arthritis, Rheumatism, Rheumatoid arthritis, Adult Still's disease, Juvenile arthritis, Early rheumatoid arthritis, Palindromic rheumatism, Gout, Infectious Arthritis, Myotonic dystrophy, Dermatomyositis, Hemophilic arthropathy, Osteopenia, Sjogren's syndrome, Juvenile Idiopathic Arthritis, Myasthenia gravis, Osteolysis, Inflammation, Degenerative polyarthritis, Osteitis deformans, Pigmented villonodular synovitis, or Hyperphosphatasemia with bone disease. In some embodiments, a musculoskeletal disorder listed in FIG. 5, 6, 7, 8, or 9 is treated with a CRISPR pharmaceutical composition targeting a gene listed as associated with the disorder in FIG. 5, 6, 7, 8, or 9. For example, in some embodiments, Rheumatoid Arthritis is treated with a CRISPR pharmaceutical composition targeting a TNFRSF1A, TNFRSF1B, TNFRSF3, TNFRSF4, or TNFRSF11A gene.

In some embodiments, the tumor necrosis factor receptor superfamily gene is an TNFRSF4 gene. In some embodiments, the TNFRSF4 gene is a human TNFRSF4 gene and the guide RNA comprises a crRNA sequence selected from SEQ ID NOs: 2867-3041. In some embodiments, editing of the TNFRSF4 gene results in a knockout of the TNFRSF4 gene. In some such embodiments, the TNFRSF4 gene is a human TNFRSF4 gene and the guide RNA comprises a crRNA sequence selected from SEQ ID NOs: 2867-2950. In some embodiments, editing of the TNFRSF4 gene results in a gene that expresses a membrane-bound receptor decoy protein. In some such embodiments, the TNFRSF4 gene is a human TNFRSF4 gene and the guide RNA comprises a crRNA sequence selected from SEQ ID NOs: 2951-2981. In some embodiments, editing of the TNFRSF4 gene results in a gene that expresses a soluble receptor decoy protein. In some such embodiments, the TNFRSF4 gene is a human TNFRSF4 gene and the guide RNA comprises a crRNA sequence selected from SEQ ID NOs: 2982-3035.

### 2. Treatment of Neoplasia by Targeting a Tumor Necrosis Factor Receptor Superfamily Gene

In one aspect, methods and pharmaceutical compositions are provided for treating a neoplasia using a CRISPR pharmaceutical composition that targets a Tumor Necrosis Factor Receptor Superfamily gene. In some embodiments, the CRISPR pharmaceutical composition comprises (i) an RNA-guided nuclease or a nucleic acid encoding an RNA-guided nuclease, and (ii) at least one guide RNA (gRNA), or a nucleic acid encoding at least one gRNA, targeting the Tumor Necrosis Factor Receptor Superfamily gene, where the gRNA specifically binds a target sequence that is adjacent to a protospacer adjacent motif (PAM) sequence for the RNA-guided nuclease. In some embodiments, the CRISPR pharmaceutical composition comprises (i) an mRNA encoding an RNA-guided nuclease, and (ii) at least one gRNA targeting the Tumor Necrosis Factor Receptor Superfamily gene. In some embodiments, the mRNA encoding the RNA-guided nuclease and the at least one gRNA are packaged in a lipid nanoparticle (LNP). In some embodiments, the neoplasia is Neoplasms, Glioblastoma, Astrocytoma, Adenocarcinoma, Osteosarcoma, Squamous cell carcinoma, Metastasis, Ameloblastoma, Cholangiocarcinoma, Choriocarcinoma, Ovarian cancer, Prostate cancer, Lung Cancer, Larynx Cancer, Breast Cancer, Lip and Oral Cavity Carcinoma, Squamous intraepithelial lesion, Neuroblastoma, Non-Hodgkin lymphomas, Malignant Neoplasms, Skin Neoplasms, Neuroblastoma, Neoplasm Metastasis, Colorectal Cancer, Fibrosarcoma, Myeloid Leukemia, Myelofibrosis, Hodgkin Lymphomas, Non-Small Cell Lung Carcinoma, Cervical Cancer, Liver cancer, Extrapulmonary small cell carcinoma, Basal cell carcinoma, Kidney cancer, Pancreatic carcinoma, Mesothelioma, Gastric cancer, Polycystic Ovary Syndrome, Chordoma, Cholangiocarcinoma, Malignant ascites, Nasopharyngeal carcinoma, Head and Neck Carcinoma, Cancer metastasis, Prostate carcinoma, Fibrosarcoma, Liver carcinoma, Carcinoma of bladder, T-Cell Lymphoblastic Leukemia, Bladder Neoplasm, melanoma, Leukemia (such as acute myeloid leukemia), Hepatocellular carcinoma, Colorectal cancer, or Lymphoma (such as Mycosis fungoides and Sézary syndrome). In some embodiments, a neoplasia listed in FIG. 5, 6, 7, 8, or 9 is treated with a CRISPR pharmaceutical composition targeting a gene listed as associated with the disorder in FIG. 5, 6, 7, 8, or 9. For example, in some embodiments, Glioblastoma is treated with a CRISPR pharmaceutical composition targeting a TNFRSF1A, TNFRSF1B, TNFRSF3, TNFRSF4, or TNFRSF11A gene.

In some embodiments, the tumor necrosis factor receptor superfamily gene is an TNFRSF4 gene. In some embodiments, the TNFRSF4 gene is a human TNFRSF4 gene and the guide RNA comprises a crRNA sequence selected from SEQ ID NOs: 2867-3041. In some embodiments, editing of the TNFRSF4 gene results in a knockout of the TNFRSF4 gene. In some such embodiments, the TNFRSF4 gene is a human TNFRSF4 gene and the guide RNA comprises a crRNA sequence selected from SEQ ID NOs: 2867-2950. In some embodiments, editing of the TNFRSF4 gene results in a gene that expresses a membrane-bound receptor decoy protein. In some such embodiments, the TNFRSF4 gene is a human TNFRSF4 gene and the guide RNA comprises a crRNA sequence selected from SEQ ID NOs: 2951-2981. In some embodiments, editing of the TNFRSF4 gene results in a gene that expresses a soluble receptor decoy protein. In some such embodiments, the TNFRSF4 gene is a human TNFRSF4 gene and the guide RNA comprises a crRNA sequence selected from SEQ ID NOs: 2982-3035.

### 3. Treatment of Autoimmune or Inflammatory Disorders by Targeting a Tumor Necrosis Factor Receptor Superfamily Gene

In one aspect, methods and pharmaceutical compositions are provided for treating an autoimmune or inflammatory disorder using a CRISPR pharmaceutical composition that targets a Tumor Necrosis Factor Receptor Superfamily gene. In some embodiments, the CRISPR pharmaceutical composition comprises (i) an RNA-guided nuclease or a nucleic acid encoding an RNA-guided nuclease, and (ii) at least one guide RNA (gRNA), or a nucleic acid encoding at least one gRNA, targeting the Tumor Necrosis Factor Receptor Superfamily gene, where the gRNA specifically binds a target sequence that is adjacent to a protospacer adjacent motif (PAM) sequence for the RNA-guided nuclease. In some embodiments, the CRISPR pharmaceutical composition comprises (i) an mRNA encoding an RNA-guided nuclease, and (ii) at least one gRNA targeting the Tumor Necrosis Factor Receptor Superfamily gene. In some embodiments, the mRNA encoding the RNA-guided nuclease and the at least one gRNA are packaged in a lipid nanoparticle (LNP). In some embodiments, the autoimmune or inflammatory disorder is Allergy, Asthma, Multiple sclerosis, Psoriasis, Inflammatory bowel disease, Autoimmune experimental uveitis, Colitis, Hypersensitivity reaction disease, Diabetes, Crohn's disease, Systemic lupus erythematosus, Pulmonary sarcoidosis, Leishmaniasis, Experimental autoimmune encephalomyelitis, HTLV-1-associated myelopathy, Tropical spastic paraparesis, Scleroderma, or Idiopathic inflammatory myopathy (such as polymyositis and dermatomyositis). In some embodiments, an autoimmune or inflammatory disorder listed in FIG. 5, 6, 7, 8, or 9 is treated with a CRISPR pharmaceutical composition targeting a gene listed as associated with the disorder in FIG. 5, 6, 7, 8, or 9. For example, in some embodiments, allergies are treated with a CRISPR pharmaceutical composition targeting a TNFRSF1A, TNFRSF1B, TNFRSF3, TNFRSF4, or TNFRSF11A gene.

In some embodiments, the tumor necrosis factor receptor superfamily gene is an TNFRSF4 gene. In some embodiments, the TNFRSF4 gene is a human TNFRSF4 gene and the guide RNA comprises a crRNA sequence selected from SEQ ID NOs: 2867-3041. In some embodiments, editing of the TNFRSF4 gene results in a knockout of the TNFRSF4 gene. In some such embodiments, the TNFRSF4 gene is a human TNFRSF4 gene and the guide RNA comprises a crRNA sequence selected from SEQ ID NOs: 2867-2950. In some embodiments, editing of the TNFRSF4 gene results in a gene that expresses a membrane-bound receptor decoy protein. In some such embodiments, the TNFRSF4 gene is a human TNFRSF4 gene and the guide RNA comprises a crRNA sequence selected from SEQ ID NOs: 2951-2981. In some embodiments, editing of the TNFRSF4 gene results in a gene that expresses a soluble receptor decoy protein. In some such embodiments, the TNFRSF4 gene is a human TNFRSF4 gene and the guide RNA comprises a crRNA sequence selected from SEQ ID NOs: 2982-3035.

### K. TNFRSF11A

In some embodiments, the pharmaceutical composition comprising a CRISPR gene-editing system targets TNFRSF11A. In some embodiments, the CRISPR gene-editing system targeting TNFRSF11A is delivered to a mammalian cell via viral vector. In some embodiments, the CRISPR gene-editing system targeting TNFRSF11A is delivered to a mammalian cell via an AAV vector. In some embodiments, the CRISPR gene-editing system targeting TNFRSF11A is delivered to a mammalian cell via a lentiviral vector. In some embodiments, the CRISPR gene-editing system targeting TNFRSF11A is delivered to a mammalian cell via a lipid nanoparticle. In some embodiments, the CRISPR gene-editing system targeting TNFRSF11A is delivered to a mammalian cell via a virus-like particle. In some embodiments, the CRISPR gene-editing system targeting TNFRSF11A is delivered to a mammalian cell via a liposome. In some embodiments, the CRISPR gene-editing system targeting TNFRSF11A is delivered to a mammalian cell via a lipid nanocrystal.

In various embodiments, the pharmaceutical composition comprising a CRISPR gene-editing system that targets the TNFRSF11A gene is used in a method of treating a mammal in need thereof. In some embodiments, the method treats one or more joint disease or illness or one or more back or spine conditions or disorders. In some embodiments, the method treats one or more musculoskeletal diseases, conditions, and illnesses, including, but not limited to, rheumatoid arthritis, gout, osteoarthritis, osteoporosis, intervertebral disc disease (IVDD), psoriatic arthritis, arthritis, polymyositis, proliferative synovitis, bone neoplasms, sarcoid myopathy, cortex bone disorders, idiopathic scoliosis, tendinopathy, myofibrillar myopathy, enthesis-related arthritis, ankylosing spondylitis, degenerative polyarthritis, arthropathy, osteitis deformans, prolapsed lumbar disc, polymyositis ossificans, idiopathic polymyositis, Luft Disease, adult-onset Still's Disease, osteoarthritis deformans, Bachet's Disease, and combinations thereof. In other embodiments, use of the system treats one or more neoplastic diseases, conditions, and illnesses, including, but not limited to, osteosarcoma, multiple myeloma, lymphoma (e.g., B-cell or cutaneous T-cell lymphoma), leukemia, thyroid carcinoma, glioma, renal cell carcinoma, chondrosarcoma, glioblastoma, melanoma, neuroblastoma, polycystic ovary syndrome, Kaposi sarcoma, squamous cell carcinoma, leiomyosarcoma, malignant peripheral nerve sheath tumor, Ewing's sarcoma, esophageal neoplasms, colon cancer, lung cancer, breast cancer, pancreatic cancer, stomach cancer, epithelial ovarian cancer, cholectoral cancer, liver cancer, skin cancer, prostate cancer, cervical cancer, ovarian cancer, bladder cancer, oral cavity cancer, and combinations thereof. In other embodiments, the method treats one or more inflammatory diseases, conditions, and illnesses, including, but not limited to, autoinflammatory disease (AID), cryopyrin-associated periodic syndrome (CAPS), systemic lupus erythematosus (SLE), inflammatory bowel disease (IBD) and combinations thereof. In some embodiments, the method treats one or more cardiac diseases, conditions, and illnesses, including, but not limited to, myocardial infarction, heart failure (e.g., refractory or acute decompensated heart failure), arrythmias, pericarditis (e.g., refractory idiopathic pericarditis), myocarditis, sepsis-induced cardiomyopathy, atherosclerosis, coronary artery disease and combinations thereof. In some embodiments, the method treats one or more neurological diseases, conditions, and illnesses, including, but not limited to, acute thrombotic stroke, epilepsy, multiple sclerosis, Alzheimer's Disease and combinations thereof. In some embodiments, the method treats one or more ophthalmic diseases, conditions, and illnesses, including, but not limited to, uveitis, scleritis, Sjogren asthenia, dry eye and combinations thereof. In some embodiments, the method treats one or more diseases, conditions, and illnesses, including, but not limited to, chronic kidney disease, Type 2 diabetes, gastroesophageal reflux disease (GERD), non-HP-associated peptic ulcer disease, and pulmonary fibrosis.

In some embodiments, the pharmaceutical composition comprising a CRISPR gene-editing system targets TNFRSF11A for the treatment of a joint disease or illness. In some embodiments, the pharmaceutical composition comprising a CRISPR gene-editing system targets TNFRSF11A for the treatment of osteoarthritis. In some embodiments, the pharmaceutical composition comprising a CRISPR gene-editing system targets TNFRSF11A for the treatment of gout.

In some embodiments, the pharmaceutical composition comprising a CRISPR gene-editing system targets TNFRSF11A for the treatment of a musculoskeletal disorder.

In some embodiments, the pharmaceutical composition comprising a CRISPR gene-editing system targets TNFRSF11A for the treatment of a back or spine condition or disorder. In some embodiments, the pharmaceutical composition comprising a CRISPR gene-editing system targets TNFRSF11A for the treatment of low back pain. In some embodiments, the pharmaceutical composition comprising a CRISPR gene-editing system targets TNFRSF11A for the treatment of degenerative disc disorder. In some embodiments, the pharmaceutical composition comprising a CRISPR gene-editing system targets TNFRSF11A for the treatment of internal disc disruption.

### 1. Treatment of Musculoskeletal Disorders by Targeting a Tumor Necrosis Factor Receptor Superfamily Gene

In one aspect, methods and pharmaceutical compositions are provided for treating a musculoskeletal disorder using a CRISPR pharmaceutical composition that targets a Tumor Necrosis Factor Receptor Superfamily gene. In some embodiments, the CRISPR pharmaceutical composition comprises (i) an RNA-guided nuclease or a nucleic acid encoding an RNA-guided nuclease, and (ii) at least one guide RNA (gRNA), or a nucleic acid encoding at least one gRNA, targeting the Tumor Necrosis Factor Receptor Superfamily gene, where the gRNA specifically binds a target sequence that is adjacent to a protospacer adjacent motif (PAM) sequence for the RNA-guided nuclease. In some embodiments, the CRISPR pharmaceutical composition comprises (i) an mRNA encoding an RNA-guided nuclease, and (ii) at least one gRNA targeting the Tumor Necrosis Factor Receptor Superfamily gene. In some embodiments, the mRNA encoding the RNA-guided nuclease and the at least one gRNA are packaged in a lipid nanoparticle (LNP). In some embodiments, the musculoskeletal disorder is Arthritis, Infectious Intermittent joint effusion, Ankylosing spondylitis, Arthritis, Osteoarthritis, Spondylarthrites, Plantar fasciitis, Degenerative polyarthritis, Hemophilic arthropathy, Inflammatory myopathy with abundant macrophages, Polymyositis, Tendinosis, Malignant Bone Neoplasm, Osteoporosis, Psoriatic arthritis, Rheumatism, Rheumatoid arthritis, Adult Still's disease, Juvenile arthritis, Early rheumatoid arthritis, Palindromic rheumatism, Gout, Infectious Arthritis, Myotonic dystrophy, Dermatomyositis, Hemophilic arthropathy, Osteopenia, Sjogren's syndrome, Juvenile Idiopathic Arthritis, Myasthenia gravis, Osteolysis, Inflammation, Degenerative polyarthritis, Osteitis deformans, Pigmented villonodular synovitis, or Hyperphosphatasemia with bone disease. In some embodiments, a musculoskeletal disorder listed in FIG. 5, 6, 7, 8, or 9 is treated with a CRISPR pharmaceutical composition targeting a gene listed as associated with the disorder in FIG. 5, 6, 7, 8, or 9. For example, in some embodiments, Rheumatoid Arthritis is treated with a CRISPR pharmaceutical composition targeting a TNFRSF1A, TNFRSF1B, TNFRSF3, TNFRSF4, or TNFRSF11A gene.

In some embodiments, the tumor necrosis factor receptor superfamily gene is an TNFRSF11A gene. In some embodiments, the TNFRSF11A gene is a human TNFRSF11A gene and the guide RNA comprises a crRNA sequence selected from SEQ ID NOs: 3042-3335. In some embodiments, editing of the TNFRSF11A gene results in a knockout of the TNFRSF11A gene. In some such embodiments, the TNFRSF11A gene is a human TNFRSF11A gene and the guide RNA comprises a crRNA sequence selected from SEQ ID NOs: 3042-3106. In some embodiments, editing of the TNFRSF11A gene results in a gene that expresses a membrane-bound receptor decoy protein. In some such embodiments, the TNFRSF11A gene is a human TNFRSF11A gene and the guide RNA comprises a crRNA sequence selected from SEQ ID NOs: 3107-3322. In some embodiments, editing of the TNFRSF11A gene results in a gene that expresses a soluble receptor decoy protein. In some such embodiments, the TNFRSF11A gene is a human TNFRSF11A gene and the guide RNA comprises a crRNA sequence selected from SEQ ID NOs: 3323-3331.

### 2. Treatment of Neoplasia by Targeting a Tumor Necrosis Factor Receptor Superfamily Gene

In one aspect, methods and pharmaceutical compositions are provided for treating a neoplasia using a CRISPR pharmaceutical composition that targets a Tumor Necrosis Factor Receptor Superfamily gene. In some embodiments, the CRISPR pharmaceutical composition comprises (i) an RNA-guided nuclease or a nucleic acid encoding an RNA-guided nuclease, and (ii) at least one guide RNA (gRNA), or a nucleic acid encoding at least one gRNA, targeting the Tumor Necrosis Factor Receptor Superfamily gene, where the gRNA specifically binds a target sequence that is adjacent to a protospacer adjacent motif (PAM) sequence for the RNA-guided nuclease. In some embodiments, the CRISPR pharmaceutical composition comprises (i) an mRNA encoding an RNA-guided nuclease, and (ii) at least one gRNA targeting the Tumor Necrosis Factor Receptor Superfamily gene. In some embodiments, the mRNA encoding the RNA-guided nuclease and the at least one gRNA are packaged in a lipid nanoparticle (LNP). In some embodiments, the neoplasia is Neoplasms, Glioblastoma, Astrocytoma, Adenocarcinoma, Osteosarcoma, Squamous cell carcinoma, Metastasis, Ameloblastoma, Cholangiocarcinoma, Choriocarcinoma, Ovarian cancer, Prostate cancer, Lung Cancer, Larynx Cancer, Breast Cancer, Lip and Oral Cavity Carcinoma, Squamous intraepithelial lesion, Neuroblastoma, Non-Hodgkin lymphomas, Malignant Neoplasms, Skin Neoplasms, Neuroblastoma, Neoplasm Metastasis, Colorectal Cancer, Fibrosarcoma, Myeloid Leukemia, Myelofibrosis, Hodgkin Lymphomas, Non-Small Cell Lung Carcinoma, Cervical Cancer, Liver cancer, Extrapulmonary small cell carcinoma, Basal cell carcinoma, Kidney cancer, Pancreatic carcinoma, Mesothelioma, Gastric cancer, Polycystic Ovary Syndrome, Chordoma, Cholangiocarcinoma, Malignant ascites, Nasopharyngeal carcinoma, Head and Neck Carcinoma, Cancer metastasis, Prostate carcinoma, Fibrosarcoma, Liver carcinoma, Carcinoma of bladder, T-Cell Lymphoblastic Leukemia, Bladder Neoplasm, melanoma, Leukemia (such as acute myeloid leukemia), Hepatocellular carcinoma, Colorectal cancer, or Lymphoma (such as Mycosis fungoides and Sézary syndrome). In some embodiments, a neoplasia listed in FIG. 5, 6, 7, 8, or 9 is treated with a CRISPR pharmaceutical composition targeting a gene listed as associated with the disorder in FIG. 5, 6, 7, 8, or 9. For example, in some embodiments, Glioblastoma is treated with a CRISPR pharmaceutical composition targeting a TNFRSF1A, TNFRSF1B, TNFRSF3, TNFRSF4, or TNFRSF11A gene.

In some embodiments, the tumor necrosis factor receptor superfamily gene is an TNFRSF11A gene. In some embodiments, the TNFRSF11A gene is a human TNFRSF11A gene and the guide RNA comprises a crRNA sequence selected from SEQ ID NOs: 3042-3335. In some embodiments, editing of the TNFRSF11A gene results in a knockout of the TNFRSF11A gene. In some such embodiments, the TNFRSF11A gene is a human TNFRSF11A gene and the guide RNA comprises a crRNA sequence selected from SEQ ID NOs: 3042-3106. In some embodiments, editing of the TNFRSF11A gene results in a gene that expresses a membrane-bound receptor decoy protein. In some such embodiments, the TNFRSF11A gene is a human TNFRSF11A gene and the guide RNA comprises a crRNA sequence selected from SEQ ID NOs: 3107-3322. In some embodiments, editing of the TNFRSF11A gene results in a gene that expresses a soluble receptor decoy protein. In some such embodiments, the TNFRSF11A gene is a human TNFRSF11A gene and the guide RNA comprises a crRNA sequence selected from SEQ ID NOs: 3323-3331.

### 3. Treatment of Autoimmune or Inflammatory Disorders by Targeting a Tumor Necrosis Factor Receptor Superfamily Gene

In one aspect, methods and pharmaceutical compositions are provided for treating an autoimmune or inflammatory disorder using a CRISPR pharmaceutical composition that targets a Tumor Necrosis Factor Receptor Superfamily gene. In some embodiments, the CRISPR pharmaceutical composition comprises (i) an RNA-guided nuclease or a nucleic acid encoding an RNA-guided nuclease, and (ii) at least one guide RNA (gRNA), or a nucleic acid encoding at least one gRNA, targeting the Tumor Necrosis Factor Receptor Superfamily gene, where the gRNA specifically binds a target sequence that is adjacent to a protospacer adjacent motif (PAM) sequence for the RNA-guided nuclease. In some embodiments, the CRISPR pharmaceutical composition comprises (i) an mRNA encoding an RNA-guided nuclease, and (ii) at least one gRNA targeting the Tumor Necrosis Factor Receptor Superfamily gene. In some embodiments, the mRNA encoding the RNA-guided nuclease and the at least one gRNA are packaged in a lipid nanoparticle (LNP). In some embodiments, the autoimmune or inflammatory disorder is Allergy, Asthma, Multiple sclerosis, Psoriasis, Inflammatory bowel disease, Autoimmune experimental uveitis, Colitis, Hypersensitivity reaction disease, Diabetes, Crohn's disease, Systemic lupus erythematosus, Pulmonary sarcoidosis, Leishmaniasis, Experimental autoimmune encephalomyelitis, HTLV-1-associated myelopathy, Tropical spastic paraparesis, Scleroderma, or Idiopathic inflammatory myopathy (such as polymyositis and dermatomyositis). In some embodiments, an autoimmune or inflammatory disorder listed in FIG. 5, 6, 7, 8, or 9 is treated with a CRISPR pharmaceutical composition targeting a gene listed as associated with the disorder in FIG. 5, 6, 7, 8, or 9. For example, in some embodiments, allergies are treated with a CRISPR pharmaceutical composition targeting a TNFRSF1A, TNFRSF1B, TNFRSF3, TNFRSF4, or TNFRSF11A gene.

In some embodiments, the tumor necrosis factor receptor superfamily gene is an TNFRSF11A gene. In some embodiments, the TNFRSF11A gene is a human TNFRSF11A gene and the guide RNA comprises a crRNA sequence selected from SEQ ID NOs: 3042-3335. In some embodiments, editing of the TNFRSF11A gene results in a knockout of the TNFRSF11A gene. In some such embodiments, the TNFRSF11A gene is a human TNFRSF11A gene and the guide RNA comprises a crRNA sequence selected from SEQ ID NOs: 3042-3106. In some embodiments, editing of the TNFRSF11A gene results in a gene that expresses a membrane-bound receptor decoy protein. In some such embodiments, the TNFRSF11A gene is a human TNFRSF11A gene and the guide RNA comprises a crRNA sequence selected from SEQ ID NOs: 3107-3322. In some embodiments, editing of the TNFRSF11A gene results in a gene that expresses a soluble receptor decoy protein. In some such embodiments, the TNFRSF11A gene is a human TNFRSF11A gene and the guide RNA comprises a crRNA sequence selected from SEQ ID NOs: 3323-3331.

### VIII. Administration Routes

The methods and compositions herein described encompass the use of pharmaceutical compositions comprising a CRISPR gene-editing system as an active ingredient.

Depending on the method/route of administration, pharmaceutical dosage forms come in several types. These include many kinds of liquid, solid, and semisolid dosage forms. Common pharmaceutical dosage forms include pill, tablet, or capsule, drink or syrup, and natural or herbal form such as plant or food of sorts, among many others. Notably, the route of administration (ROA) for drug delivery is dependent on the dosage form of the substance in question. A liquid pharmaceutical dosage form is the liquid form of a dose of a chemical compound used as a drug or medication intended for administration or consumption.

As described below, a composition of the present disclosure can be delivered to a subject subcutaneously (e.g., intra-articular or intradiscal injection), dermally (e.g., transdermally via patch), and/or via implant. Exemplary pharmaceutical dosage forms include, e.g., pills, osmotic delivery systems, elixirs, emulsions, hydrogels, suspensions, syrups, capsules, tablets, orally dissolving tablets (ODTs), gel capsules, thin films, adhesive topical patches, lollipops, lozenges, chewing gum, dry powder inhalers (DPIs), vaporizers, nebulizers, metered dose inhalers (MDIs), ointments, transdermal patches, intradermal implant.

As used herein, “dermal delivery” or “dermal administration” can refer to a route of administration wherein the pharmaceutical dosage form is taken to, or through, the dermis (i.e., layer of skin between the epidermis (with which it makes up the cutis) and subcutaneous tissues). “Subcutaneous delivery” can refer to a route of administration wherein the pharmaceutical dosage form is to or beneath the subcutaneous tissue layer.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, N.Y.). For example, solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Therapeutic compounds that are or include nucleic acids can be administered by any method suitable for administration of nucleic acid agents, such as a DNA vaccine. These methods include gene guns, bio injectors, and skin patches as well as needle-free methods such as the micro-particle DNA vaccine technology disclosed in U.S. Pat. No. 6,194,389, and the mammalian transdermal needle-free vaccination with powder-form vaccine as disclosed in U.S. Pat. No. 6,168,587. Additionally, intranasal delivery is possible, as described in, inter alia, Hamajima et al., Clin. Immunol. Immunopathol., 88 (2), 205-10 (1998). Liposomes (e.g., as described in U.S. Pat. No. 6,472,375) and microencapsulation can also be used. Biodegradable targetable microparticle delivery systems can also be used (e.g., as described in U.S. Pat. No. 6,471,996).

Therapeutic compounds can be prepared with carriers that will protect the therapeutic compounds against rapid elimination from the body, such as a controlled release formulation, including implants and microencapsulated delivery systems. Biodegradable, biocompatible polymers can be used, such as collagen, ethylene vinyl acetate, polyanhydrides (e.g., poly[1,3-bis(carboxyphenoxy) propane-co-sebacic-acid] (PCPP-SA) matrix, fatty acid dimer-sebacic acid (FAD-SA) copolymer, poly(lactide-co-glycolide)), polyglycolic acid, collagen, polyorthoesters, polyethylene glycol-coated liposomes, hyaluronic acid and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811. Semisolid, gelling, soft-gel, or other formulations (including controlled release) can be used, e.g., when administration to a surgical site is desired. Methods of making such formulations are known in the art and can include the use of biodegradable, biocompatible polymers. See, e.g., Sawyer et al., Yale J Biol Med. 2006 December; 79 (3-4): 141-152.

The pharmaceutical compositions described herein may be included in a container, kit, pack, or dispenser together with instructions for administration.

### A. Systemic Administration

In some embodiments, a pharmaceutical composition comprising a CRISPR gene-editing system is administered systemically to a mammal in need thereof. In some embodiments, the composition is formulated for intravenous injection. In some embodiments, the composition is formulated for oral administration. In some embodiments, the composition is formulated for parenteral administration.

### B. Local Administration

In some embodiments, a pharmaceutical composition comprising a CRISPR gene-editing system is administered locally to a mammal in need thereof. In some embodiments, the local administration is an intra-articular injection. In some embodiments, the composition is formulated for intradiscal injection. In some embodiments, the composition is formulated for epidural injection. In some embodiments, the composition is formulated for peridiscal injection. In some embodiments, the composition is formulated for perivertebral injection. In some embodiments, composition is formulated for administration to the facet joints of the spine.

In some embodiments, a pharmaceutical composition comprising a CRISPR gene-editing system is administered locally to a mammal in need thereof during a surgical procedure. In some embodiments, a pharmaceutical composition comprising a CRISPR gene-editing system is administered locally to a mammal in need thereof 1, 2, 3, 4, 5, 6, 7, 8, 9, 10, 20, or 30 days after a surgical procedure.

### EXAMPLES

The embodiments encompassed herein are now described with reference to the following examples. These examples are provided for the purpose of illustration only and the disclosure encompassed herein should in no way be construed as being limited to these examples, but rather should be construed to encompass any and all variations which become evident as a result of the teachings provided herein.

### Example 1: Design and Selection of Guides for Editing the IL1 Receptor (IL1R1)

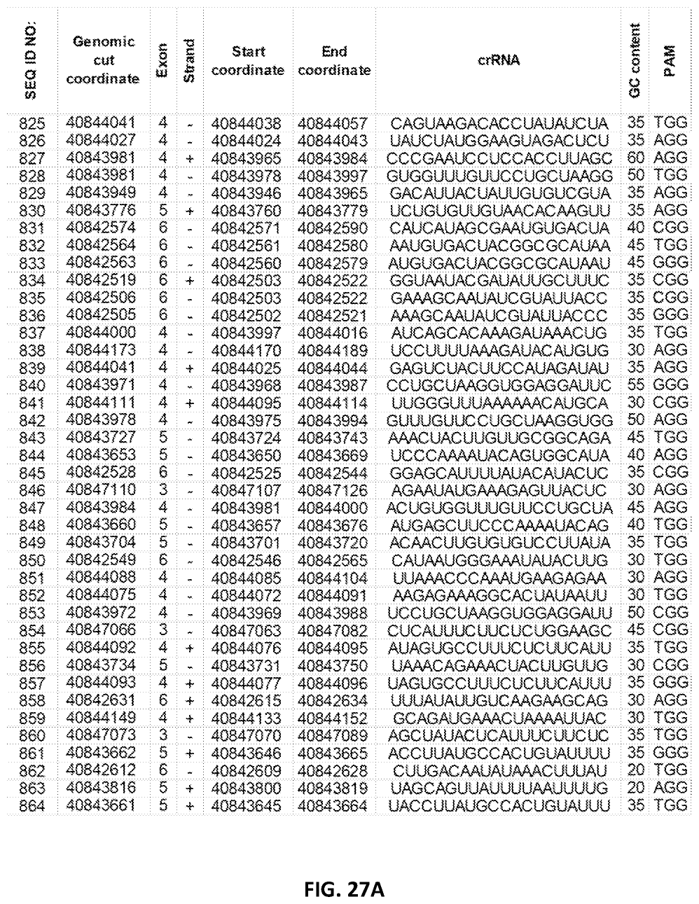

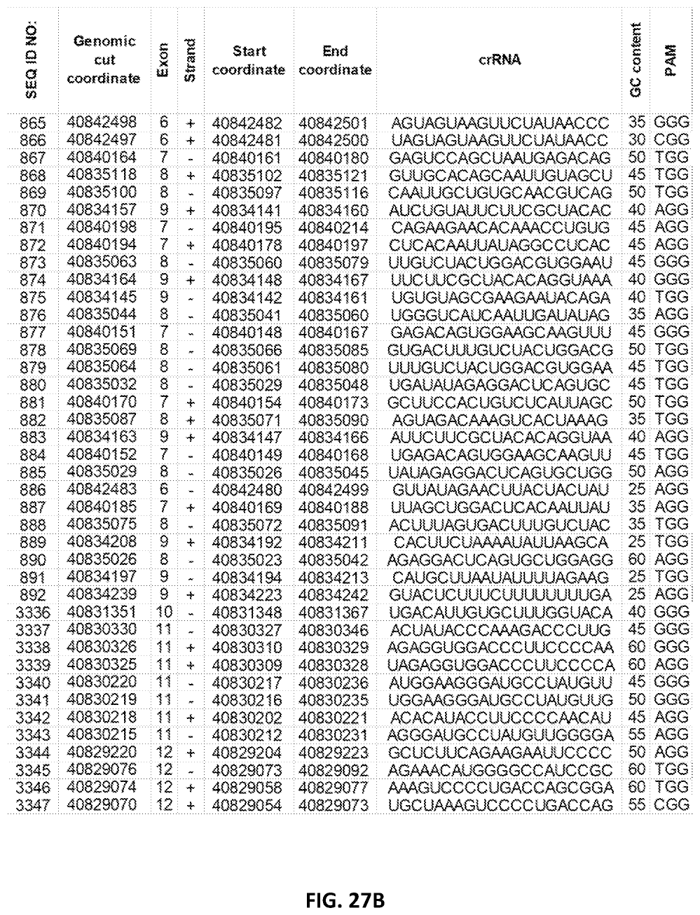

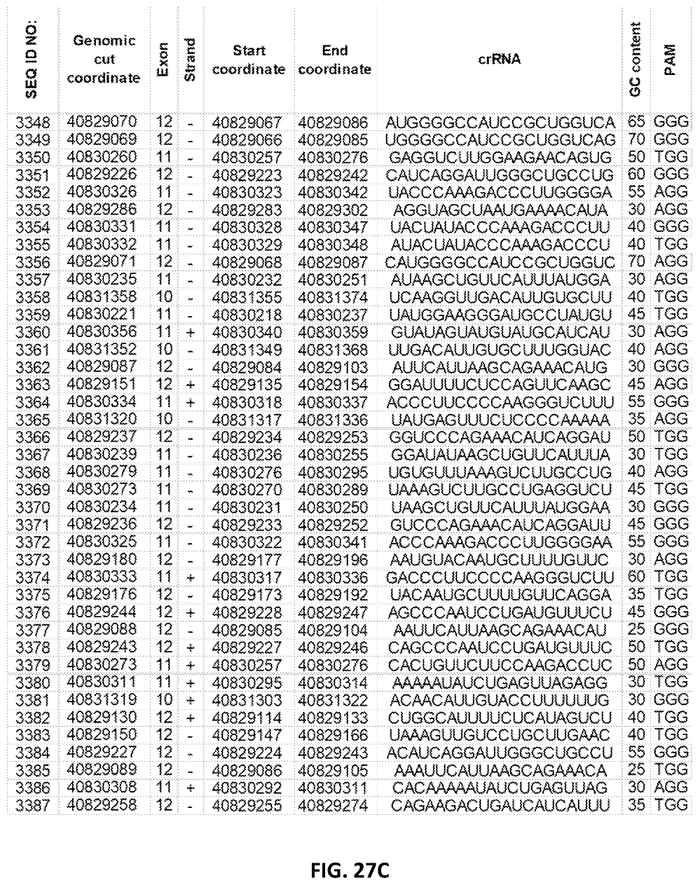

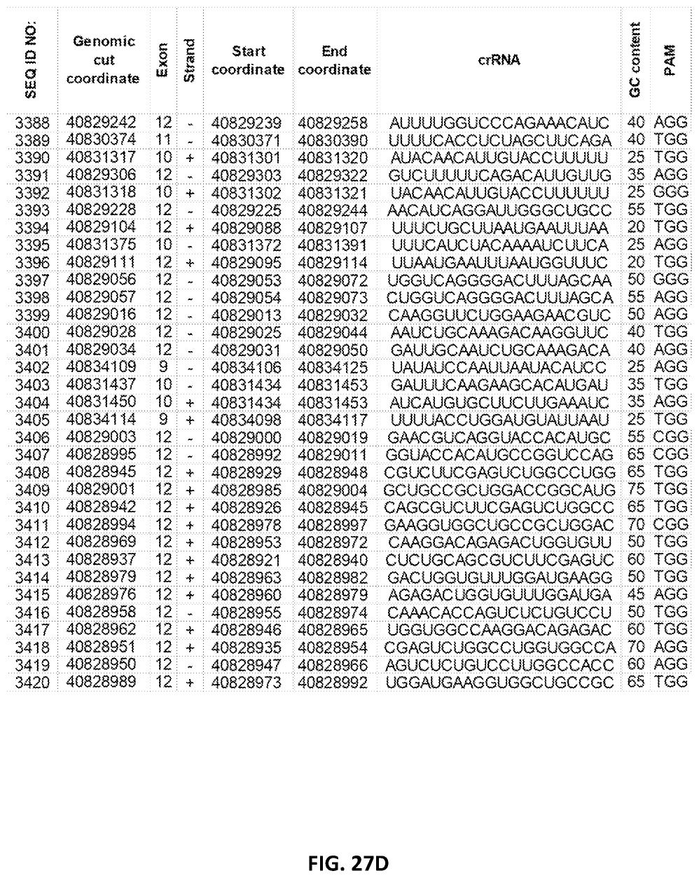

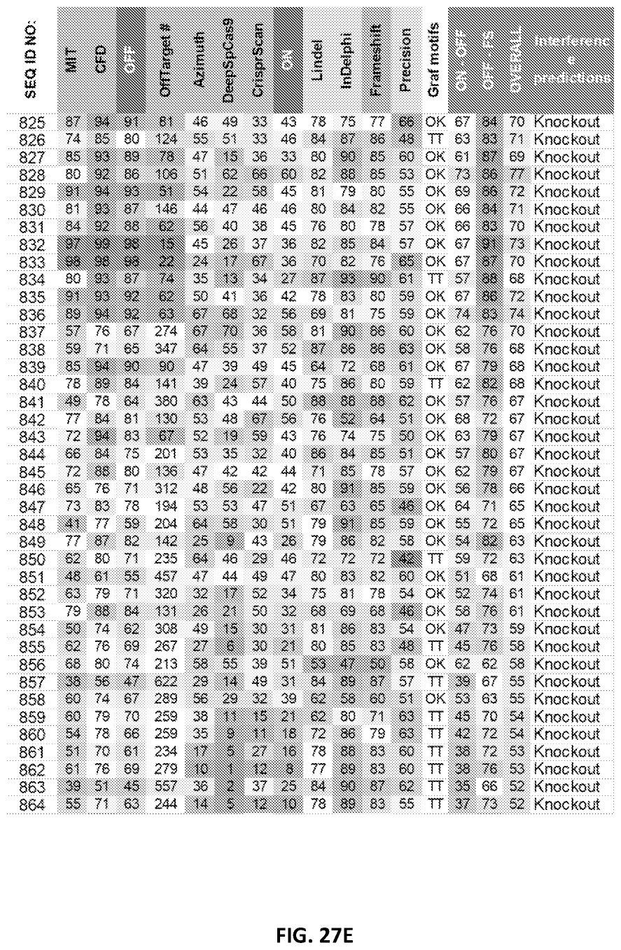

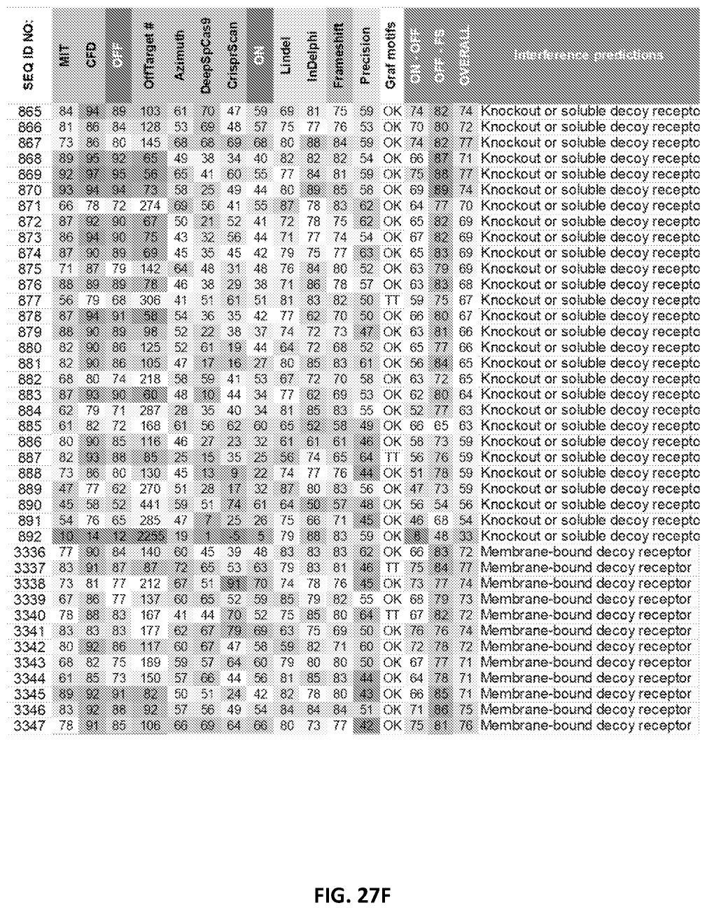

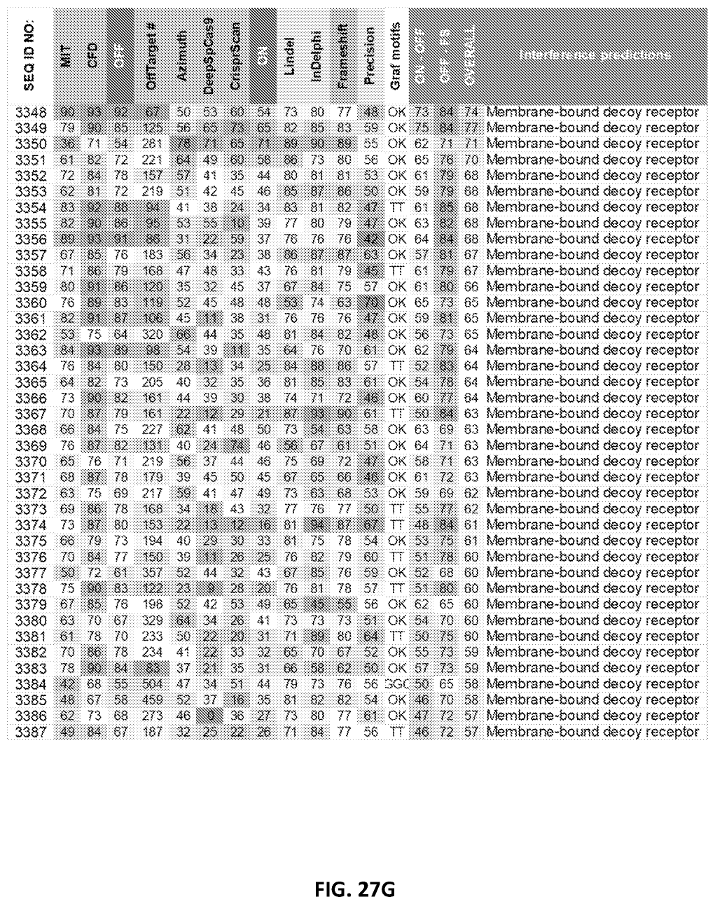

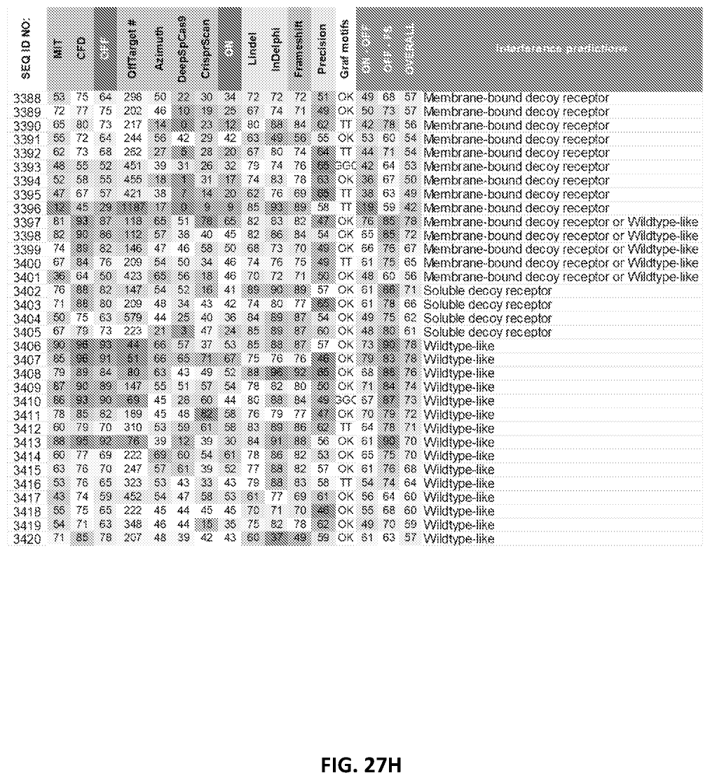

A pipeline of in silico and in vitro screening was developed to identify candidate CRISPR-Cas guides for gene editing. The pipeline was applied to identify guides that effectively edit the canine IL1R1 receptor gene (cIL1R1), thereby disrupting IL1A/IL1B signaling in vivo. The first step in the pipeline was to identify all possible crRNA sequences for a particular CRISPR-Cas protein in the coding portions of the cIL1R1. Many algorithms for identifying such sequences are known in the art. Generally, these algorithms function by identifying a protospacer adjacent motif (PAM) sequence for the particular CRISPR-Cas protein and then locate the sequence spaced according to the requirements of the particular Cas protein, typically directly 5′ of the PAM site. For example, the S. pyogenes Cas9 (SpCas9) protein, which was used in this Example, recognizes a 5′-NGG-3′ PAM sequence. Thus, all sequences directly 5′ of an NGG trinucleotide are possible crRNA sequences. crRNA sequences identified in this fashion as shown in FIG. 27, along with biographical information about the crRNA sequence.

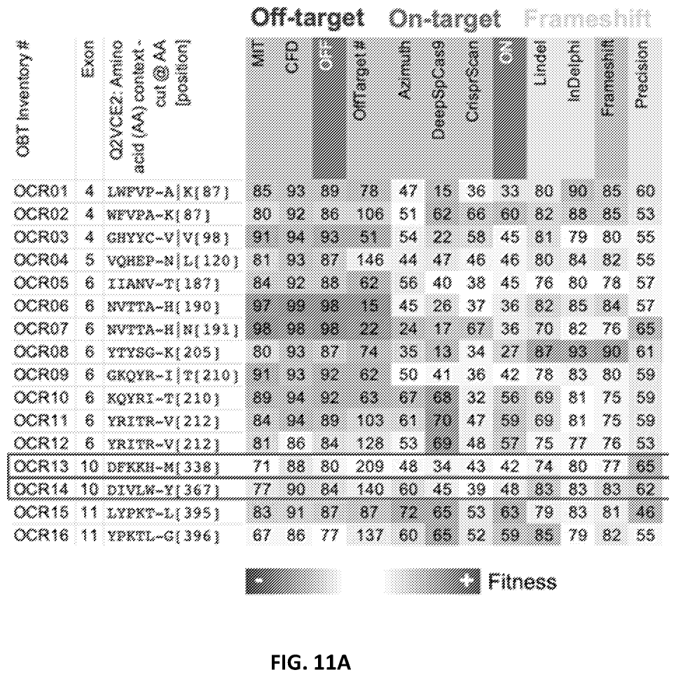

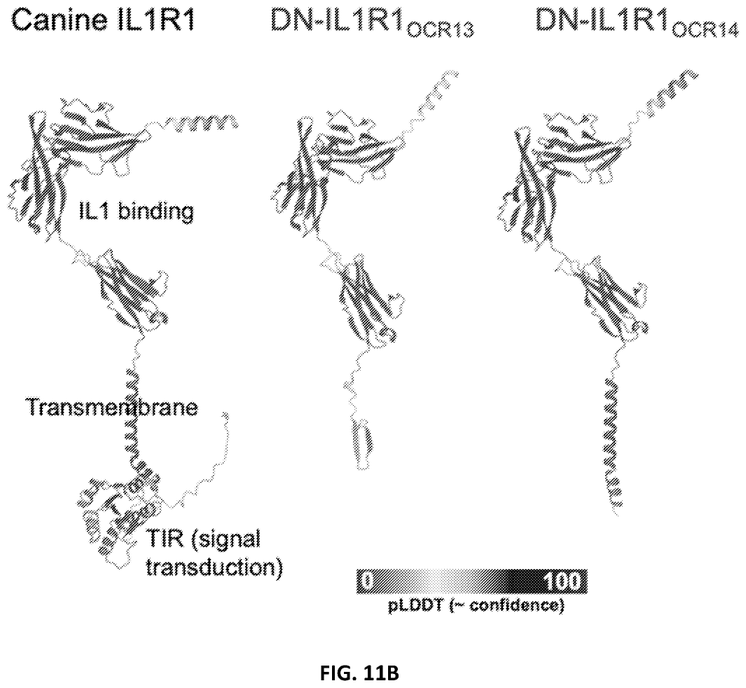

Next, each identified crRNA sequence was evaluated across three different metrics: possible off-target editing at locations in the genome other than the target gene, on-target editing efficiency, and the likelihood of editing causing frameshift mutations, using multiple algorithms for each metric, as illustrated in FIG. 11A. The basis of the combinatorial approach used lies in the assumption that every model has blind spots that may skew the fitness of a particular guide RNA. Weighting these scores to obtain a consensus score for each of these properties allows for much better prediction of sgRNA fitness.

Off-target editing effects were predicted by averaging scores generated by the MIT, CFD and Elevation (human only) model. The MIT algorithm, also known as Hsu-Zhang score. Hsu, P. D. et al. DNA targeting specificity of RNA-guided Cas9 nucleases. Nature Biotechnology 31, 827-832 (2013). This model is based on a positional penalty matrix (1×20) generated from 15 EMX1 sgRNA libraries with mismatches against target at every position. The CFD algorithm (Cutting Frequency Determination) is based on threat matrix (12×20) considering both position and mismatch type and PAM integrity (27,897 ‘CD33’ sgRNAs+10,618 negative control sgRNAs). Doench, J. G. et al. Optimized sgRNA design to maximize activity and minimize off-target effects of CRISPR-Cas9. Nature Biotechnology 34, 184-191 (2016). The Elevation score using machine learning algorithms trained by genome-wide (GUIDE-Seq) and other aggregated off-target profiling data. Listgarten, J. et al. Prediction of off-target activities for the end-to-end design of CRISPR guide RNAs. Nature Biomedical Engineering 2, 38-47 (2018). The column labelled “OFF” shows the mean of the scores provided by the two or three models, respectively. The column labelled “OffTarget #” shows the number of potential off-targets with up to four mismatches as calculated by CRISPOR. See, Haeussler, M. et al. Evaluation of off-target and on-target scoring algorithms and integration into the guide RNA selection tool CRISPOR. Genome Biology 17, 148 (2016).

On-target editing efficiencies were predicted by averaging scores generated by the Azimuth model, the DeepSpCas9 model, and the CrisprScan model. The Azimuth model is a boosted regression tree model, trained with 881 sgRNAs (MOLM13/NB4/TF1 cells and additional unpublished data) delivered by lentivirus. Doench, J. G. et al. Optimized sgRNA design to maximize activity and minimize off-target effects of CRISPR-Cas9. Nature Biotechnology 34, 184-191 (2016). DeepSpCas9 is a deep learning model trained using editing data from 12,832 sgRNA. Kim, H. K. et al. SpCas9 activity prediction by DeepSpCas9, a deep learning-based model with high generalization performance. Science Advances 5 (11) (2019). CrisprScan is a linear regression model, trained using editing data from 1000 sgRNAs injected into zebrafish embryos targeting >100 genes. Moreno-Mateos, M. A. et al. CRISPRscan: designing highly efficient sgRNAs for CRISPR-Cas9 targeting in vivo. Nature Methods 12, 982-988 (2015). The column labelled “ON” shows the mean of the scores provided by the three models.

The putative guides' potential to generate frameshift mutations were predicted by averaging scores generated by the Lindel model and the InDelphi model. Lindel is a machine learning model trained using profile data of 1.16 million independent mutational events triggered by CRISPR/Cas9-mediated cleavage and non-homologous end joining-mediated double strand break repair of 6872 synthetic target sequences, introduced into a human cell line via lentiviral infection. Chen, W. et al. Massively parallel profiling and predictive modeling of the outcomes of CRISPR/Cas9-mediated double-strand break repair. Nucleic Acids Research 47, 7989-8003 (2019). InDelphi is machine learning model trained with indels generated by 1872 sgRNAs. Shen, M. W. et al. Predictable and precise template-free CRISPR editing of pathogenic variants. Nature 563, 646-651 (2018). The column labelled “Frameshift” shows the mean of the scores provided by the two models. The column labelled “Precision” shows the frequency distribution of indels estimated by inDelphi. High precision is closer to 100 and represents sgRNAs that are characterised by one or a very low number of repair outcomes.

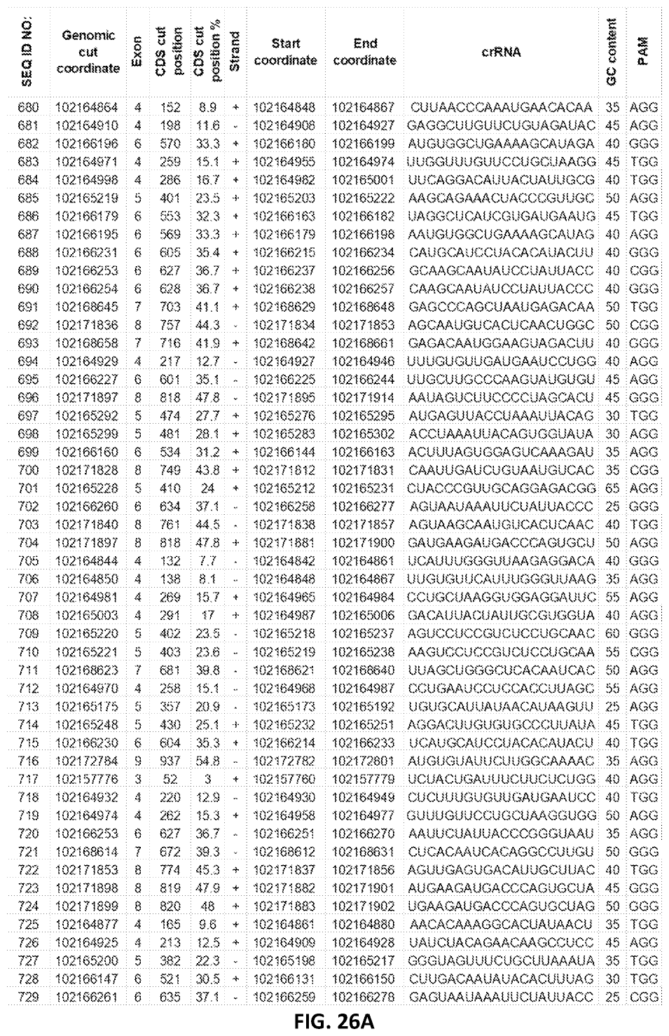

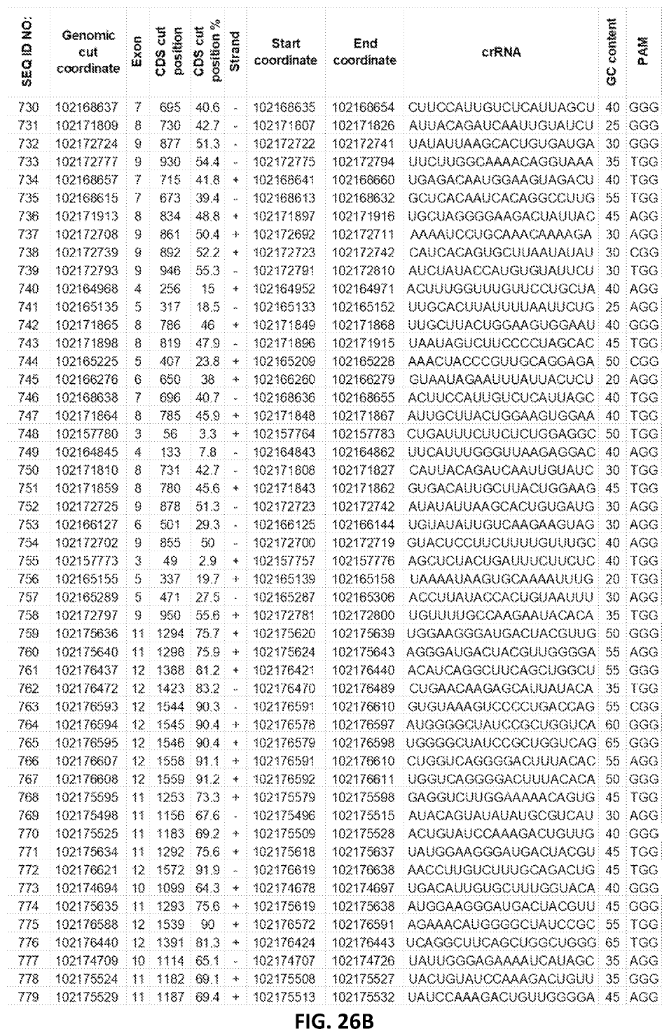

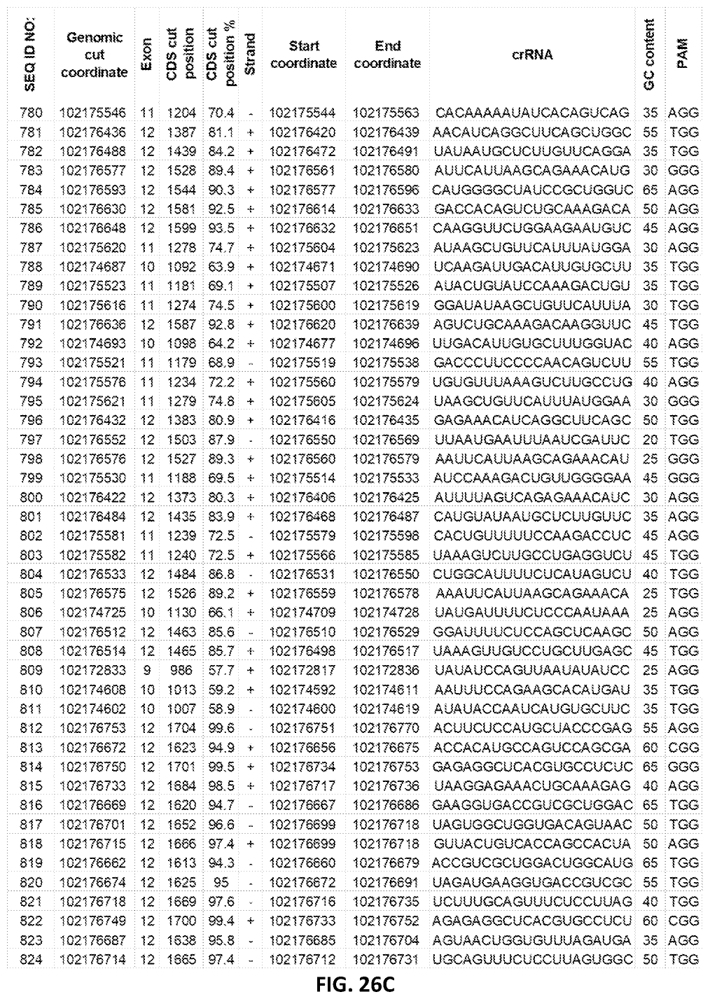

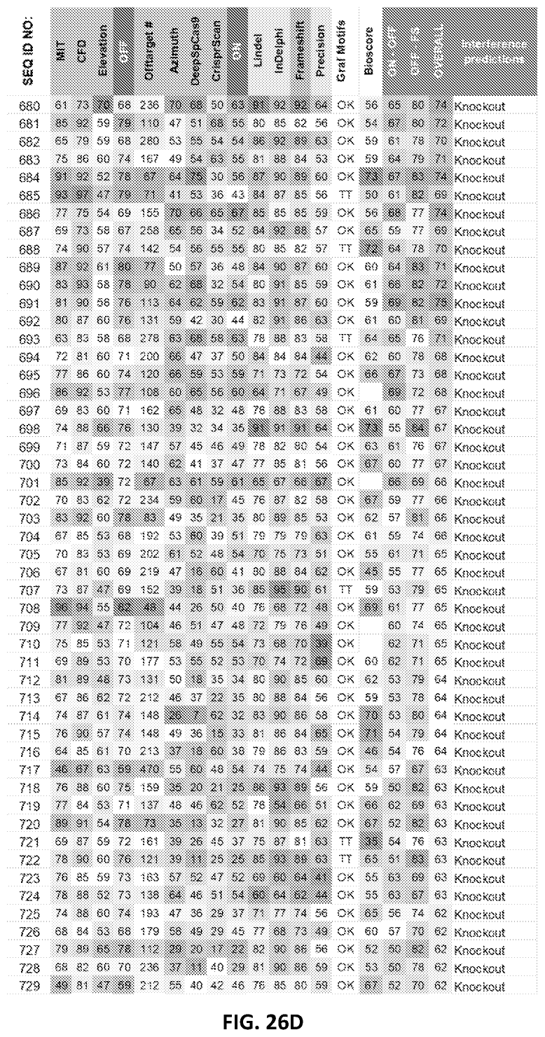

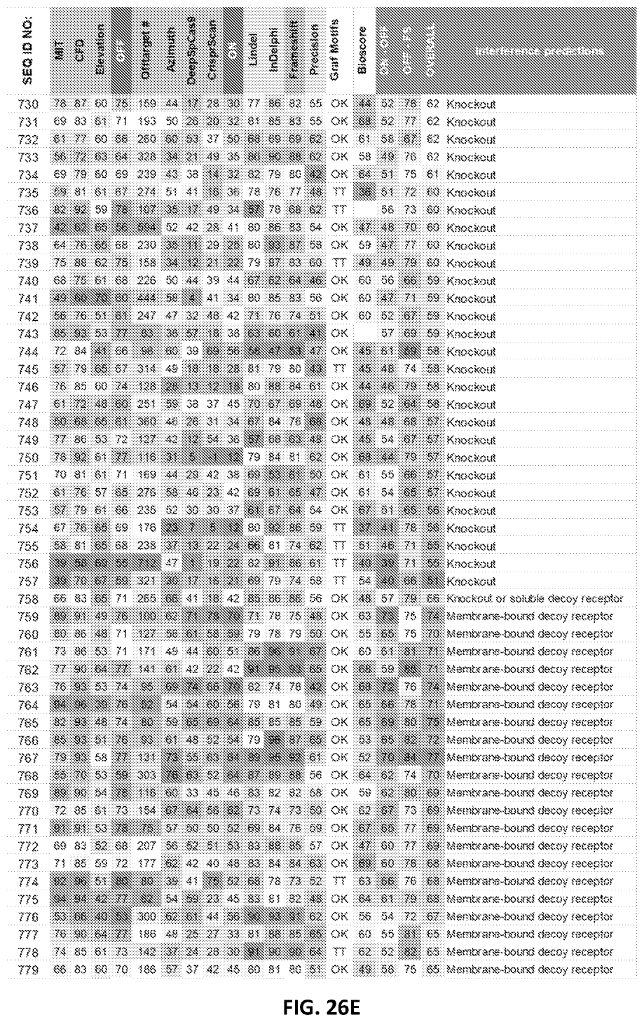

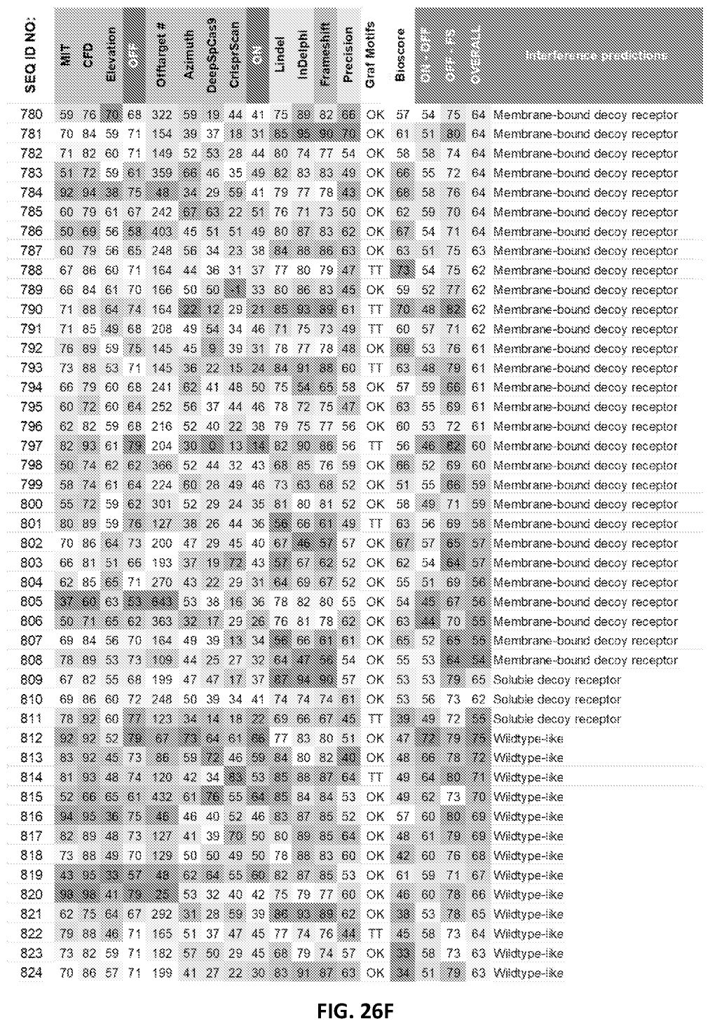

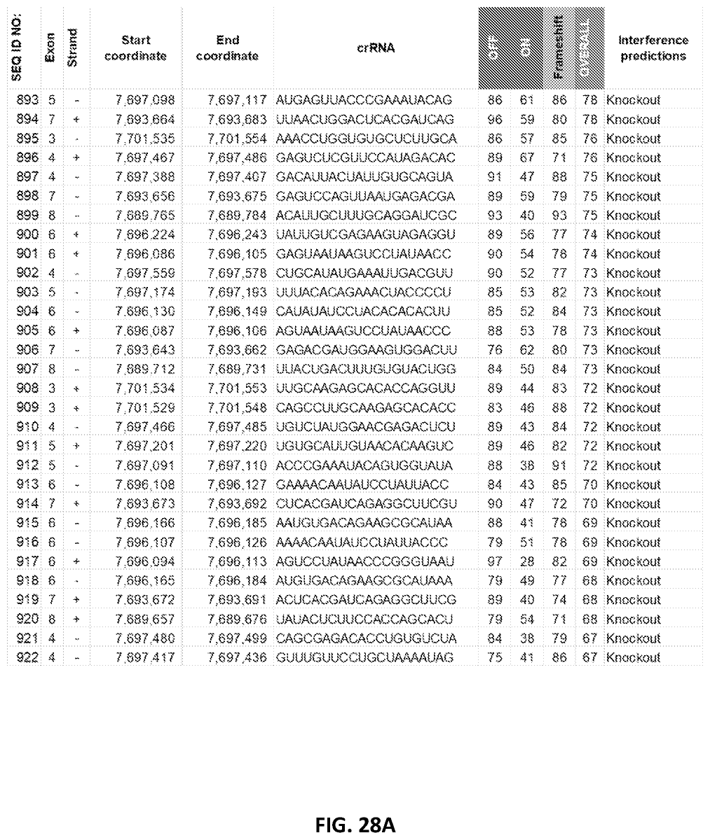

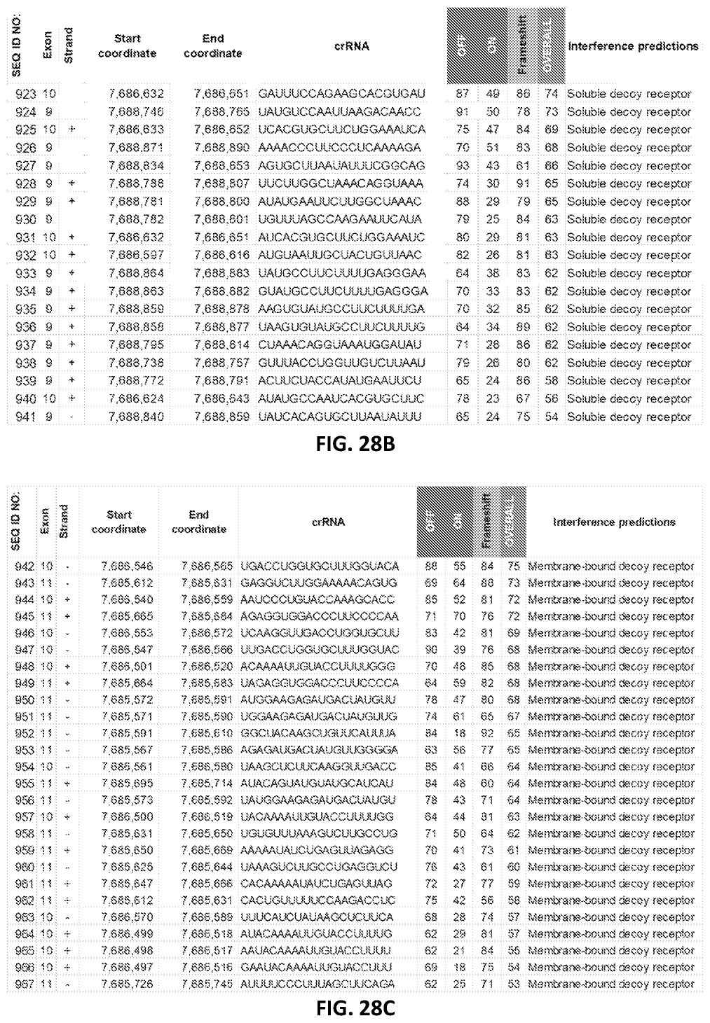

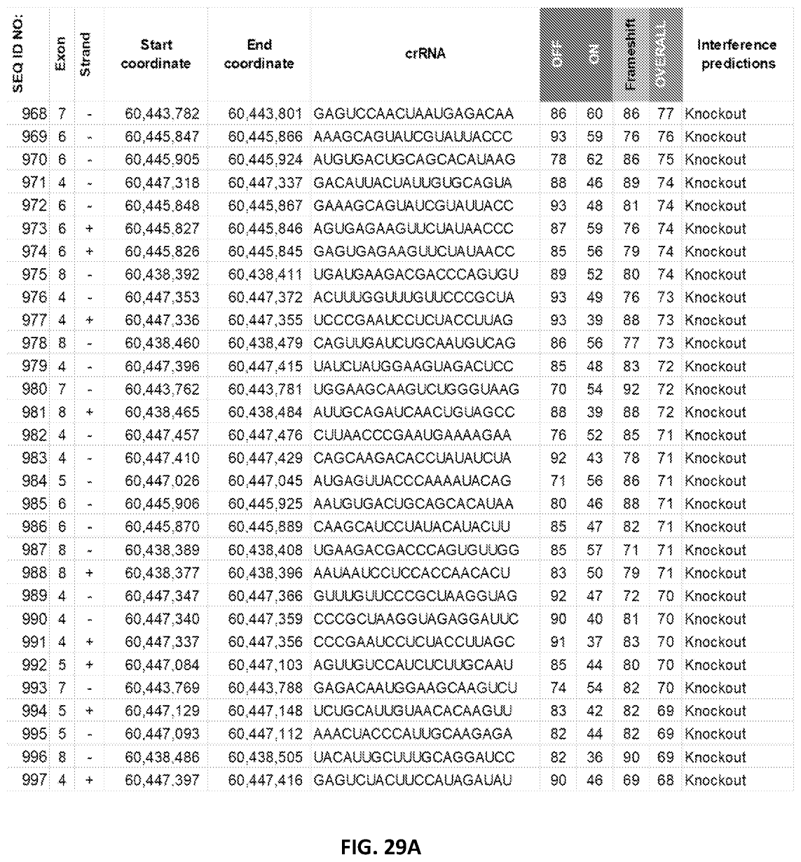

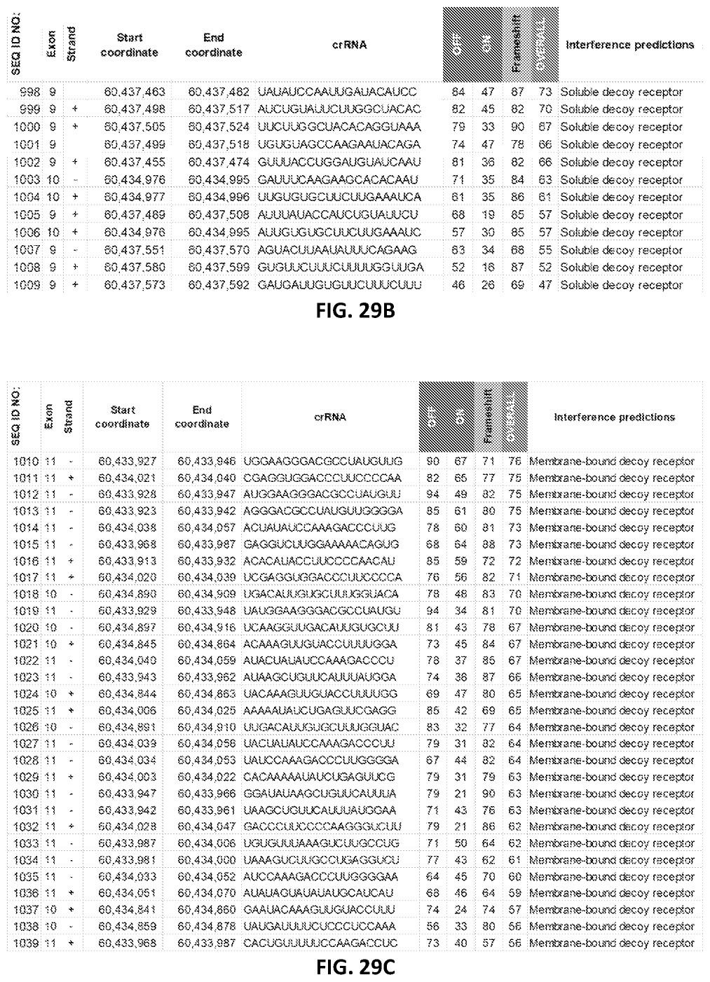

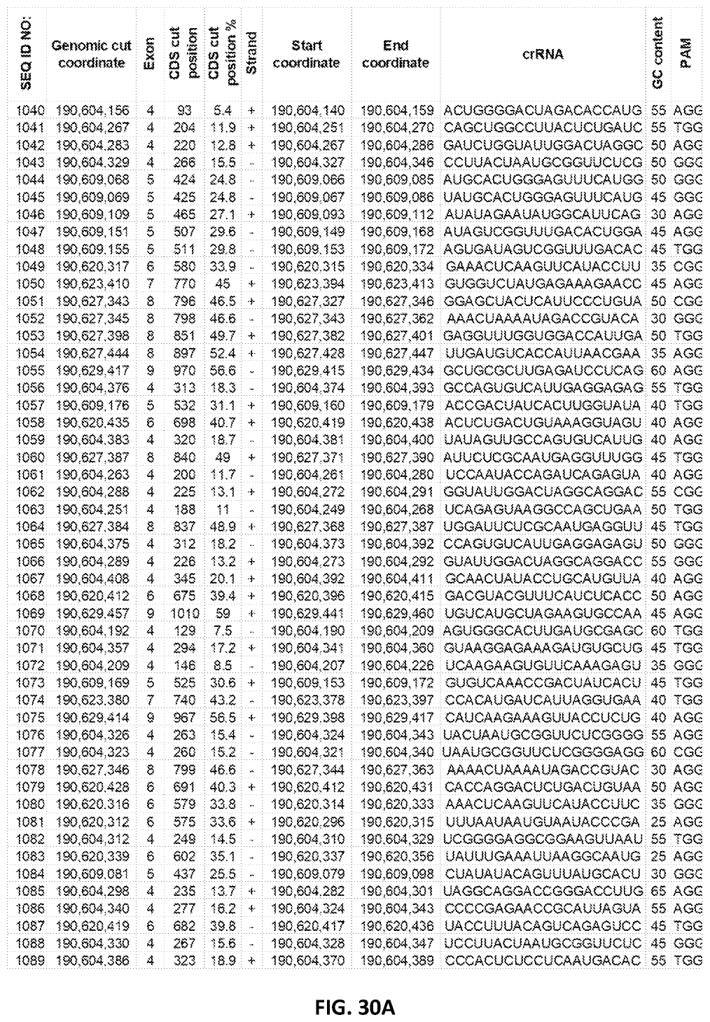

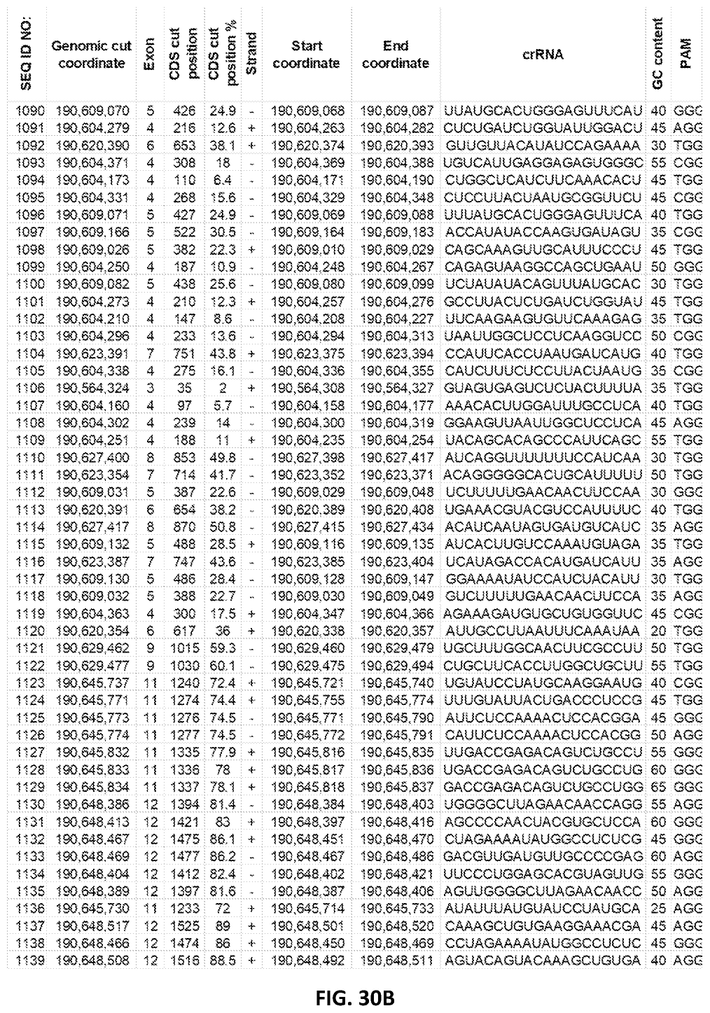

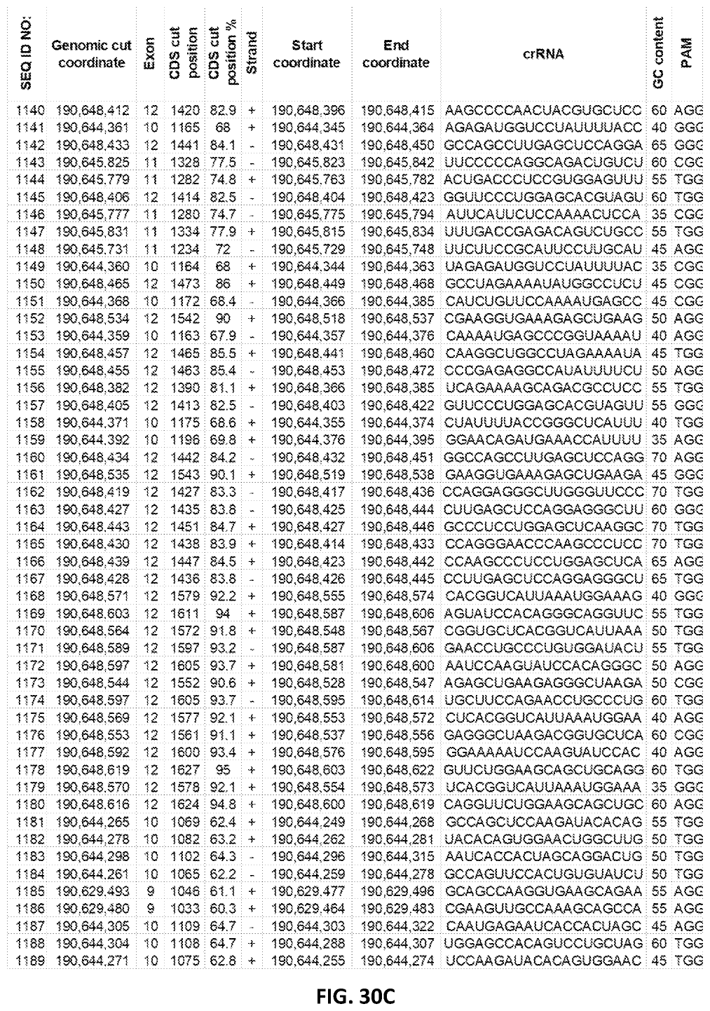

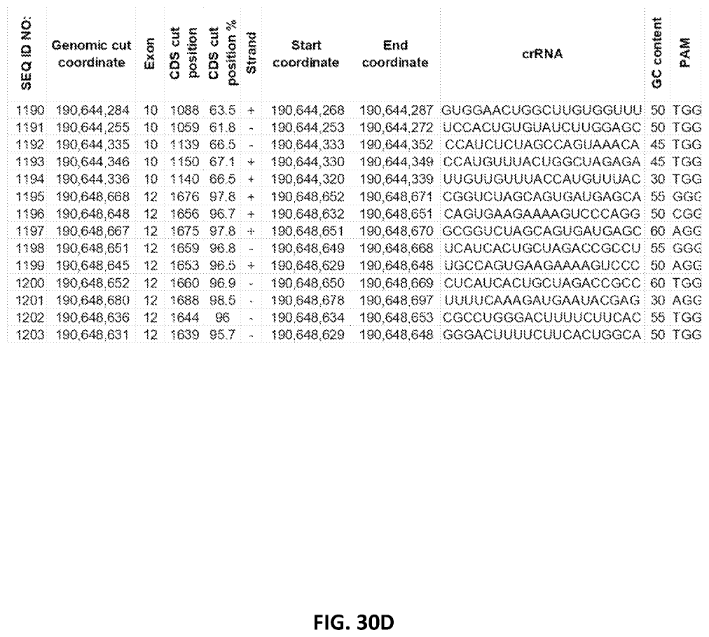

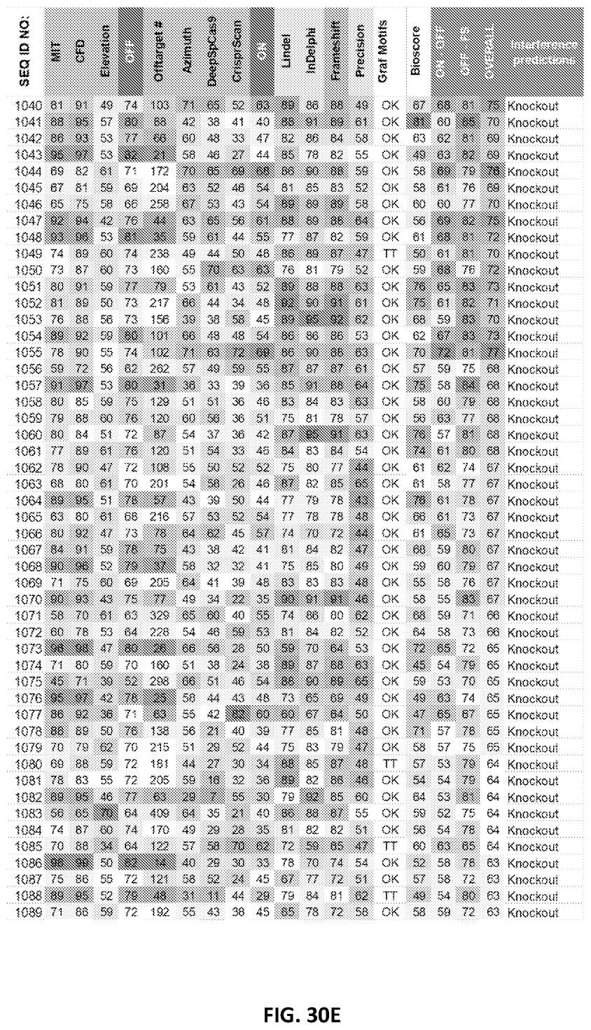

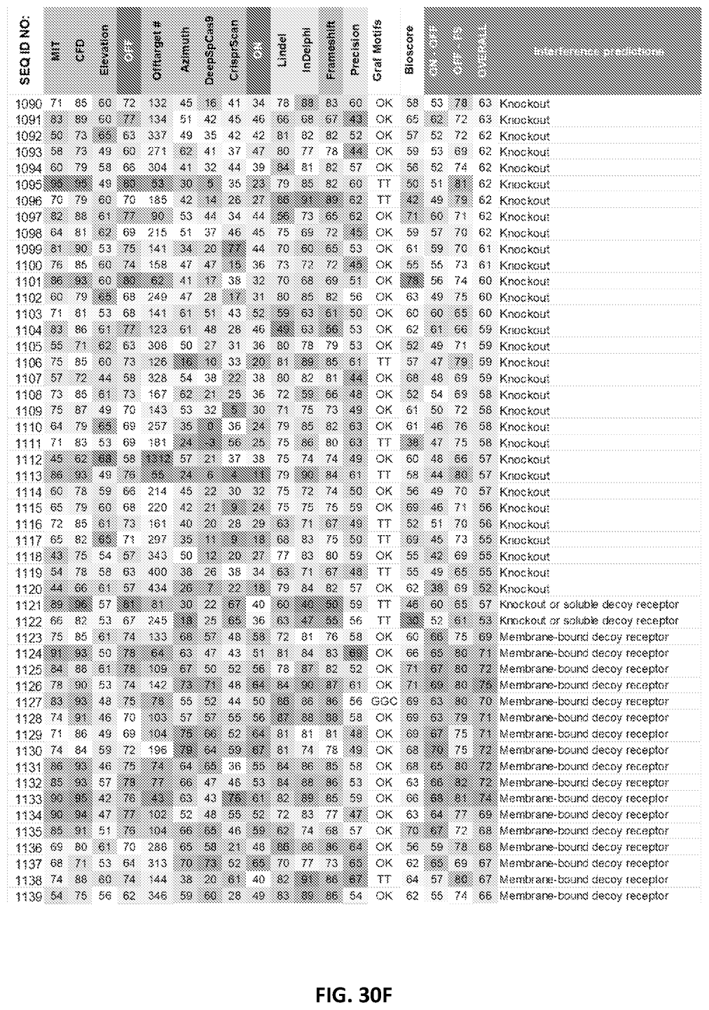

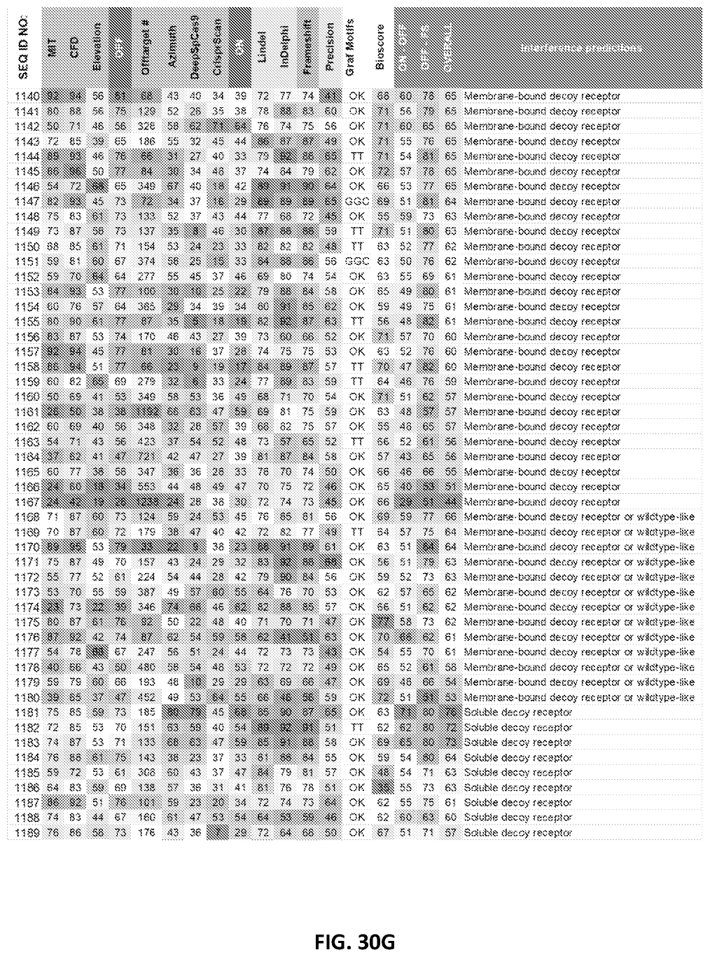

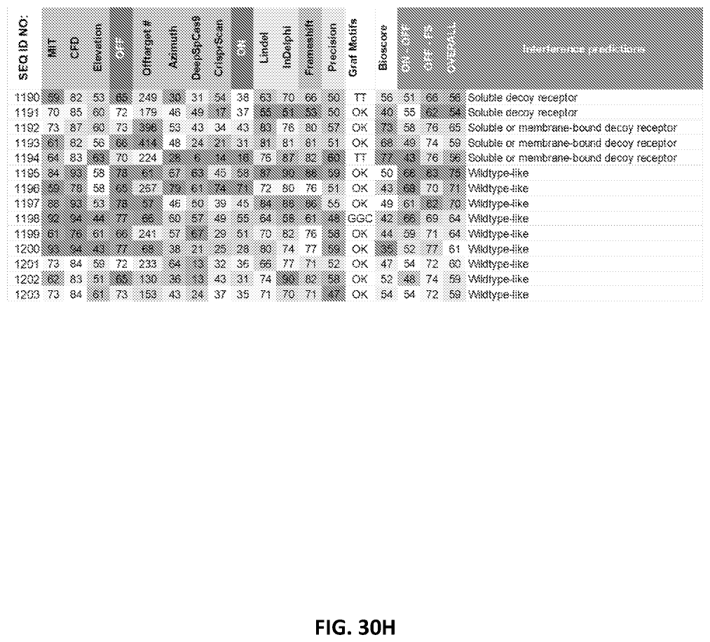

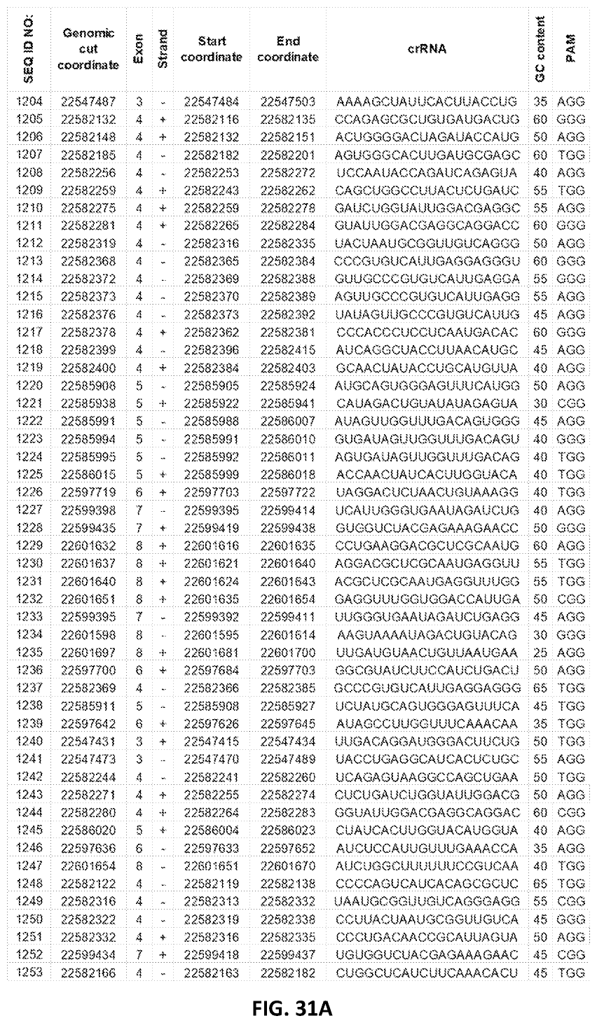

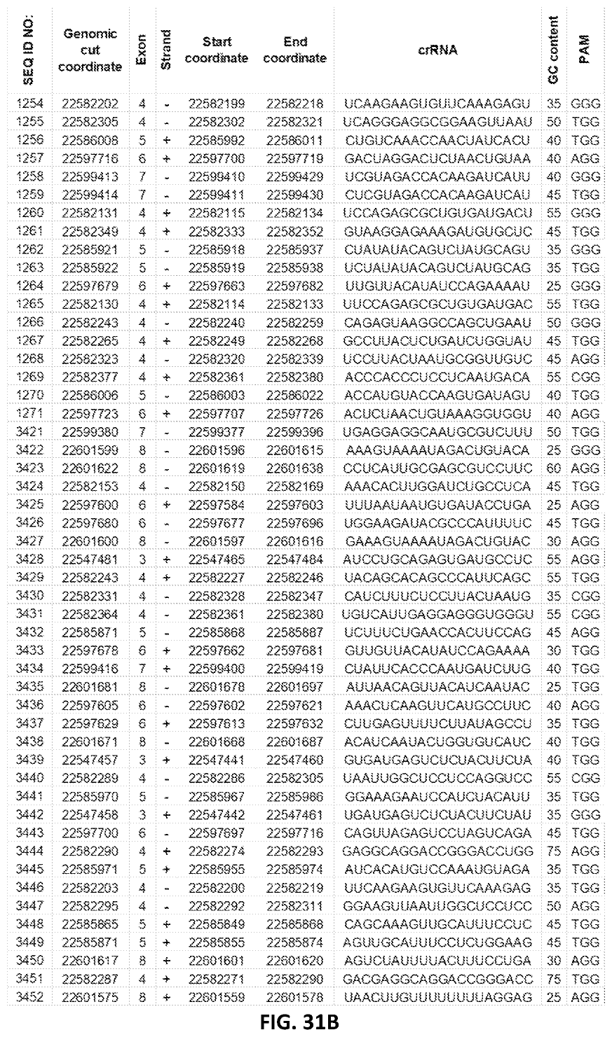

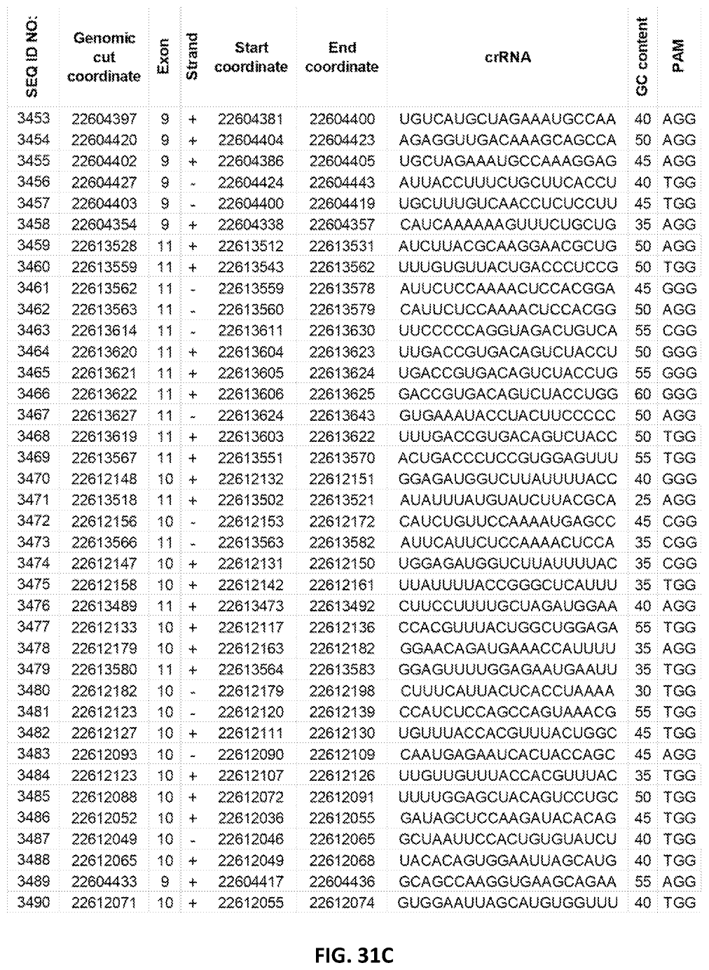

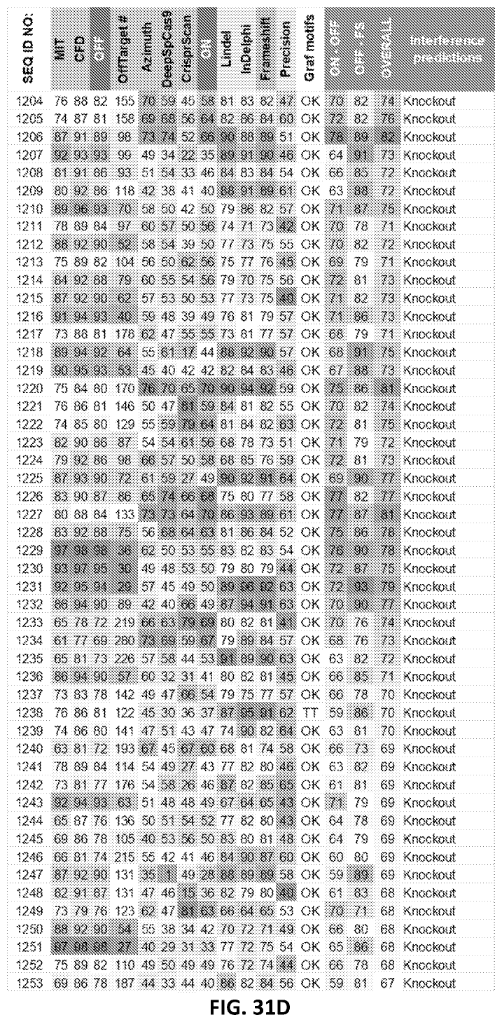

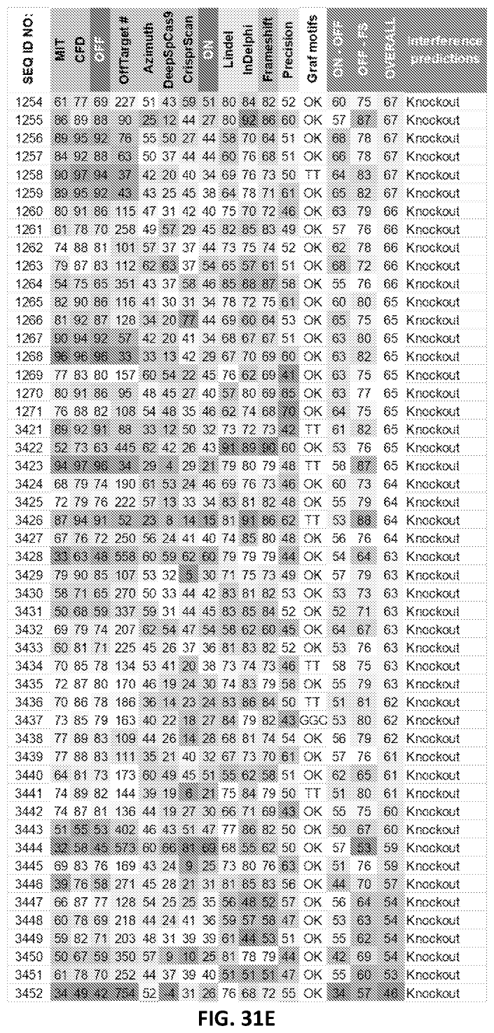

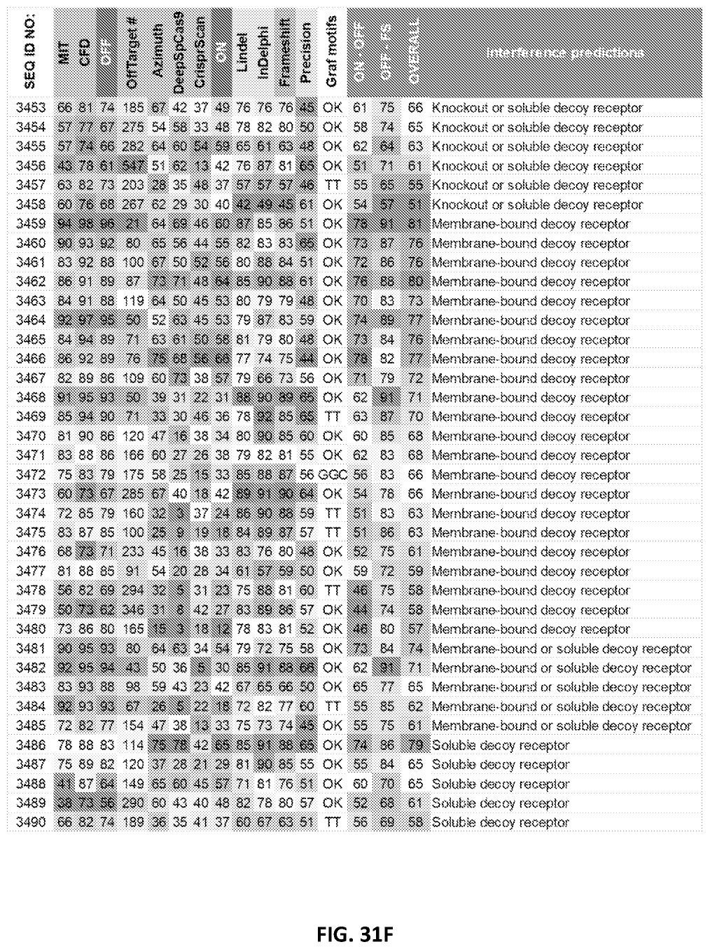

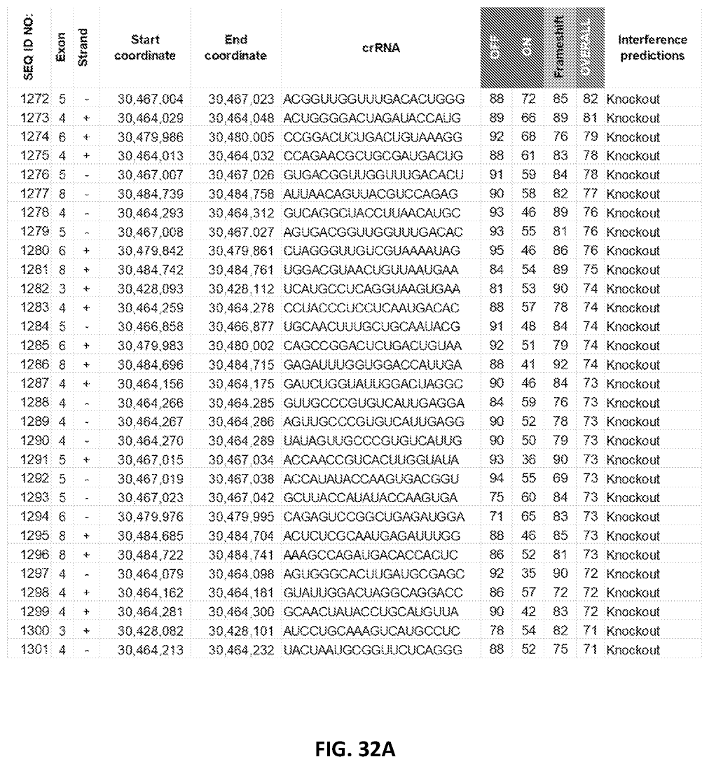

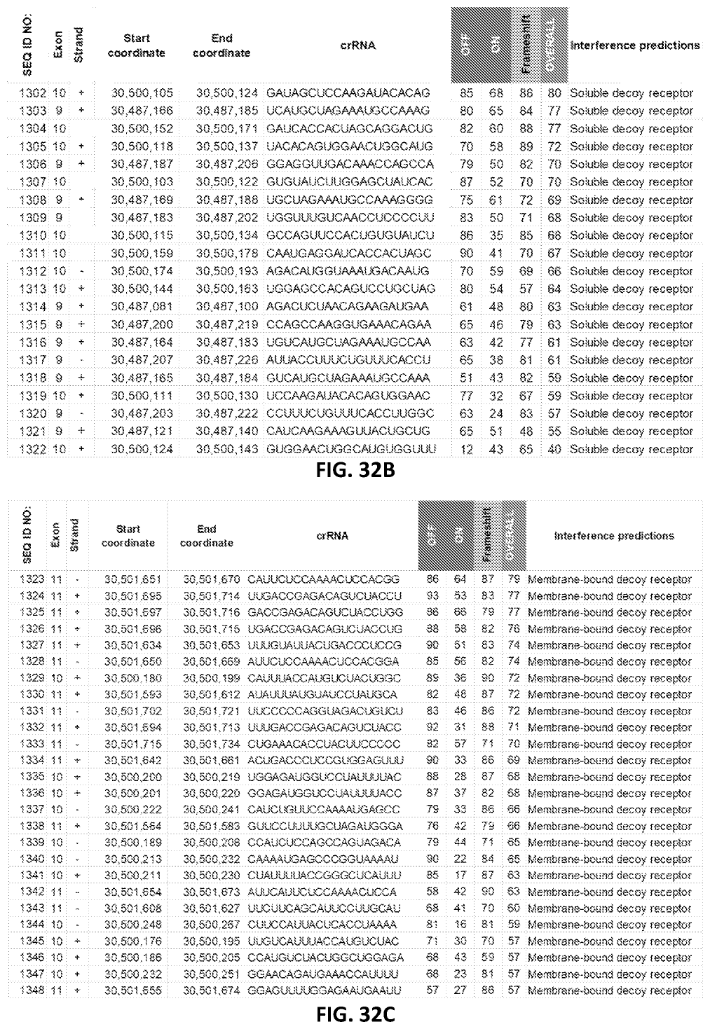

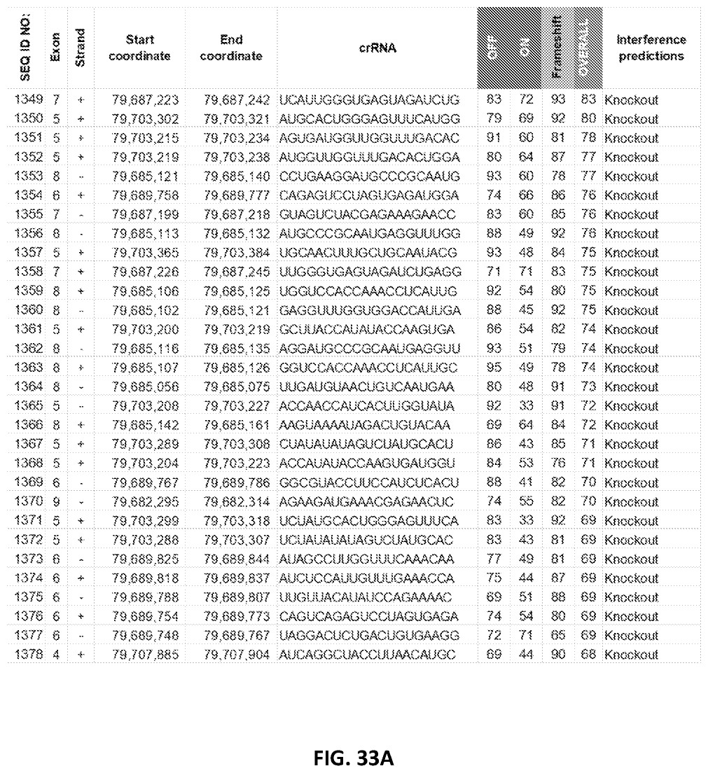

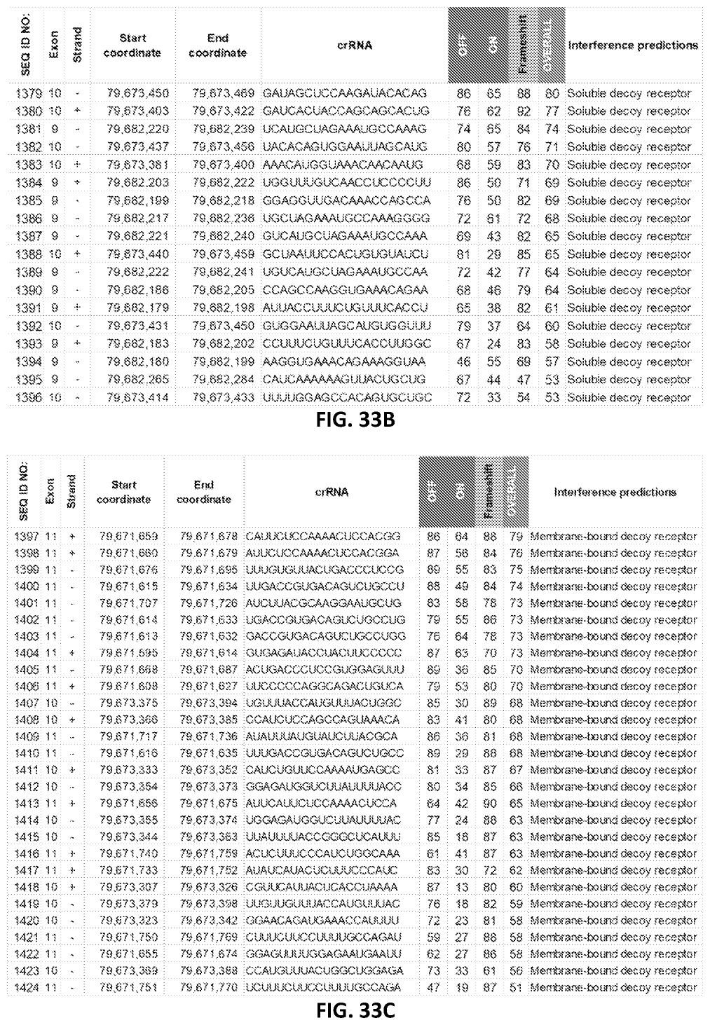

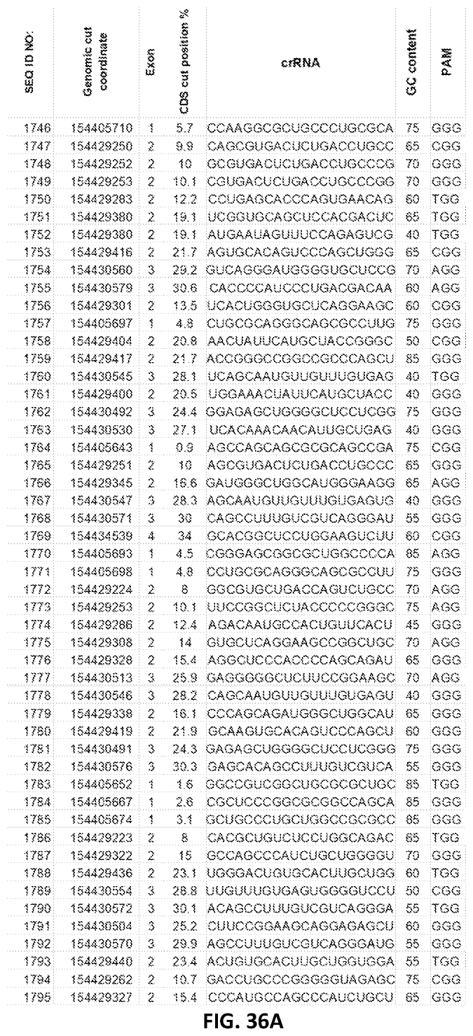

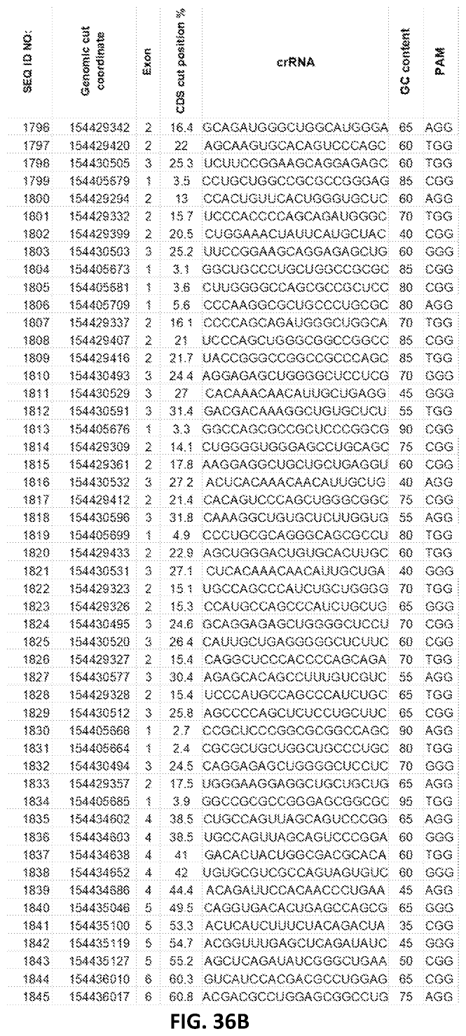

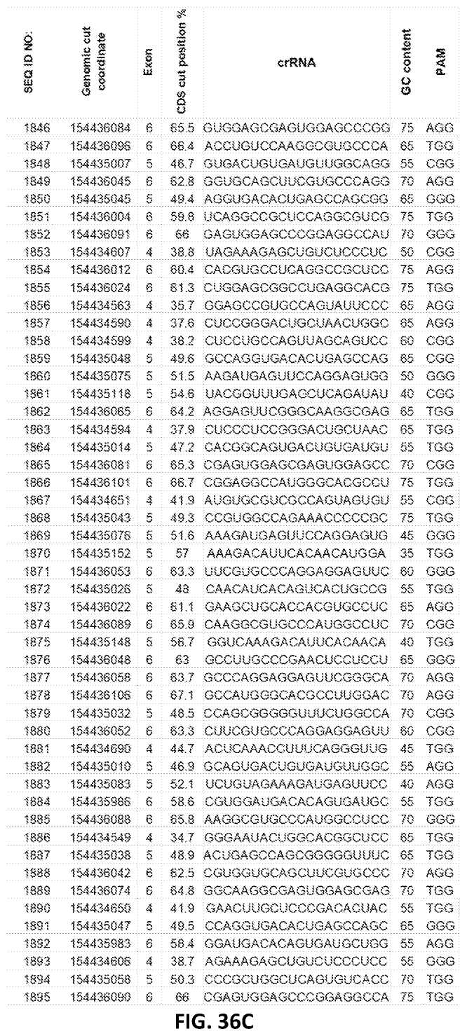

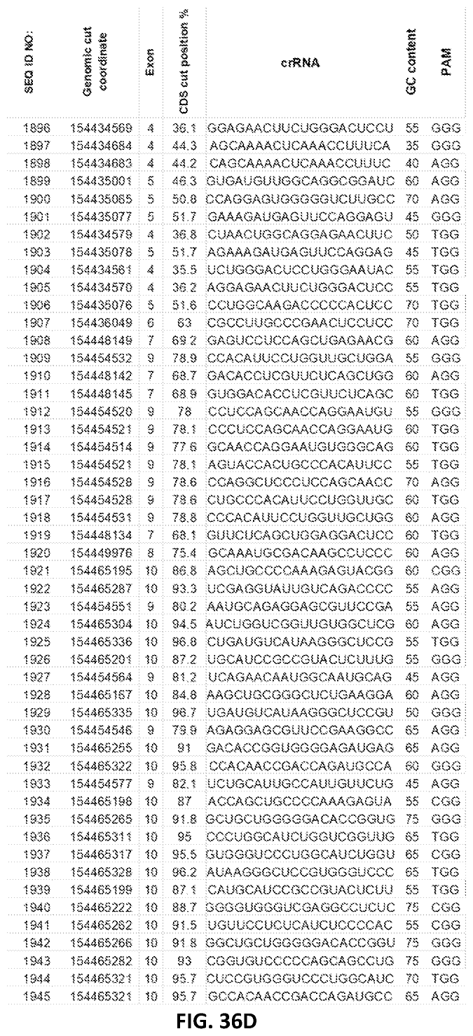

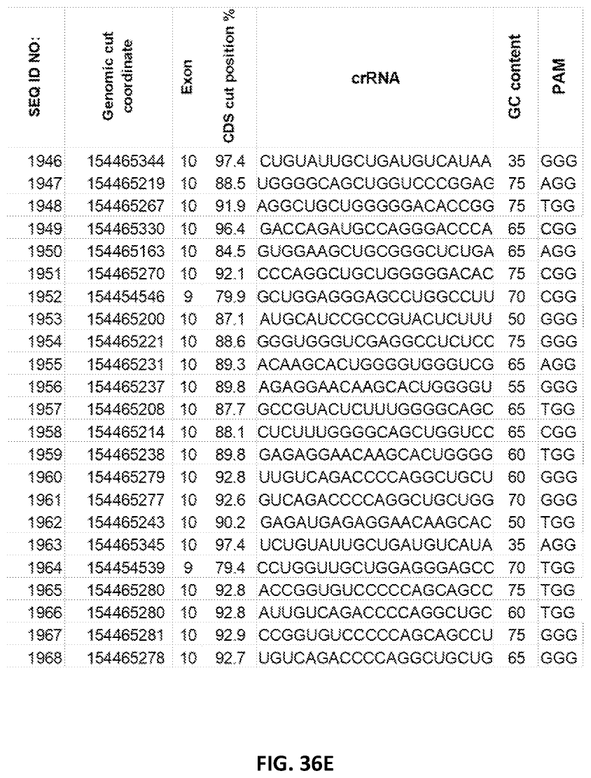

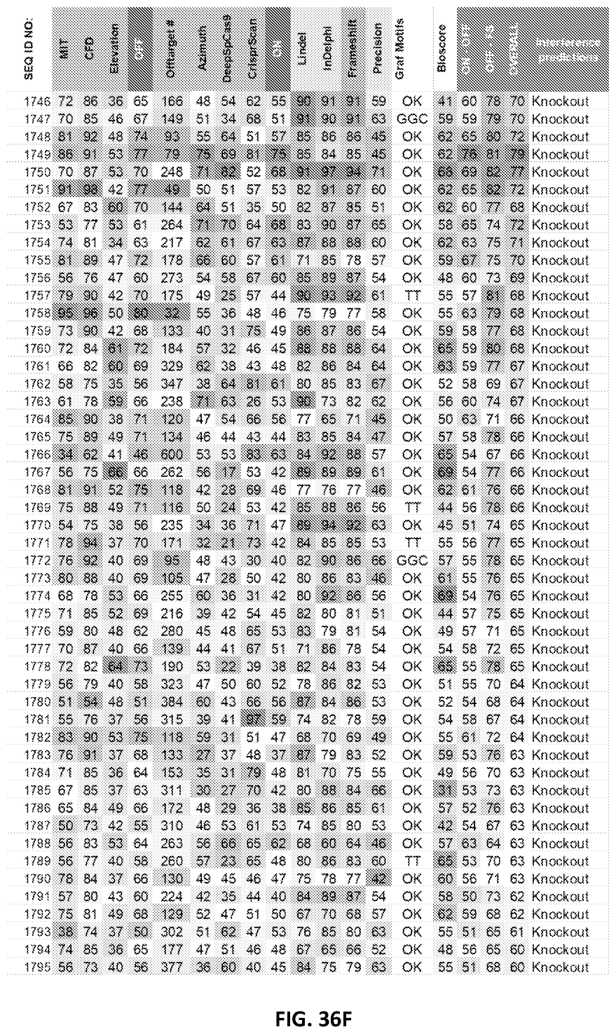

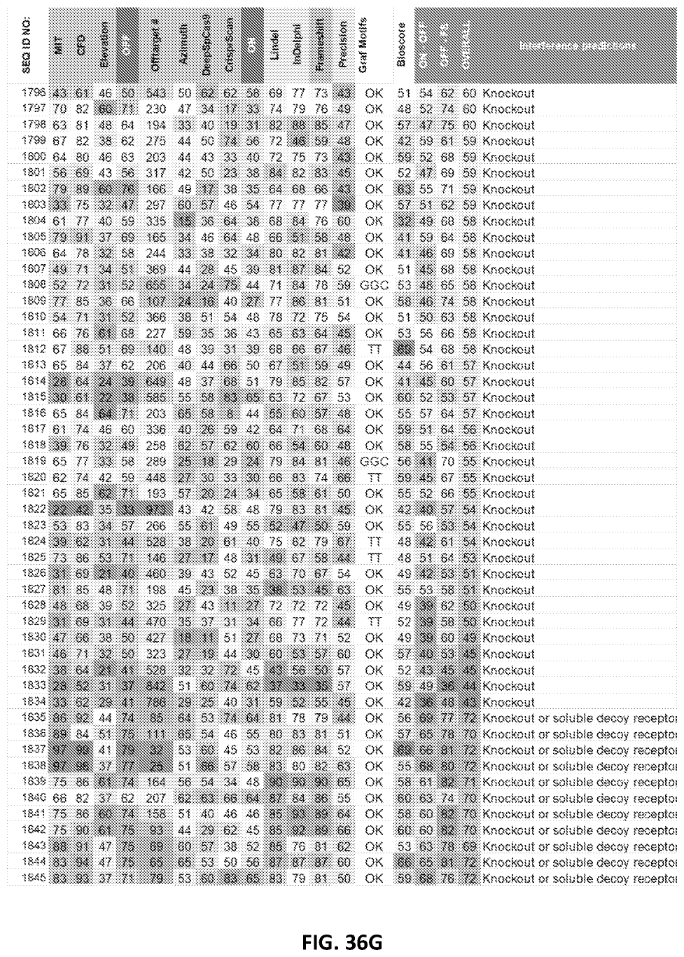

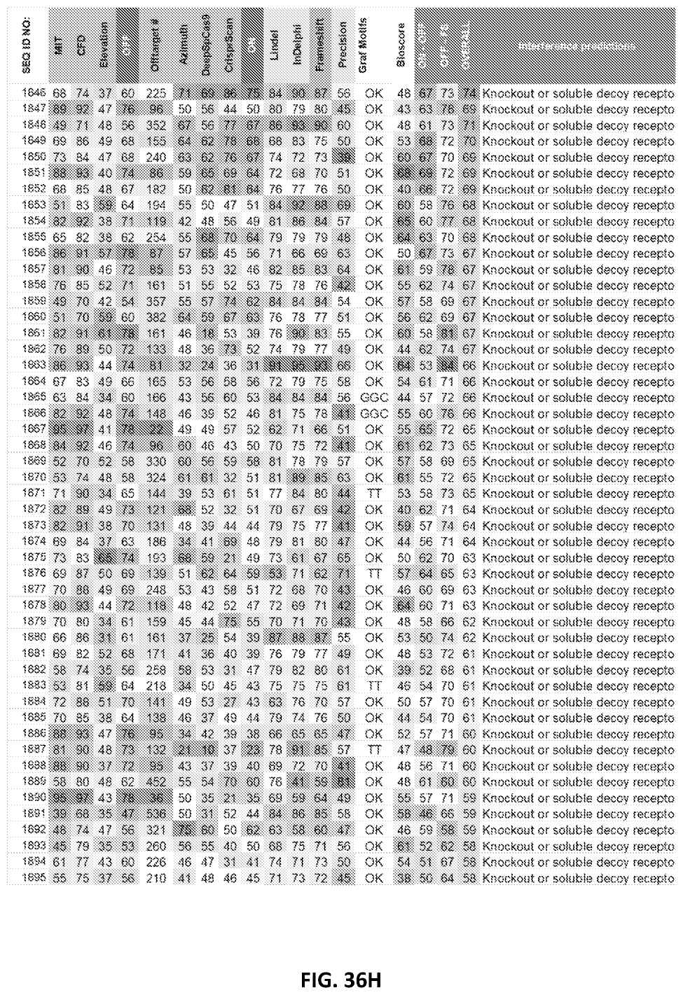

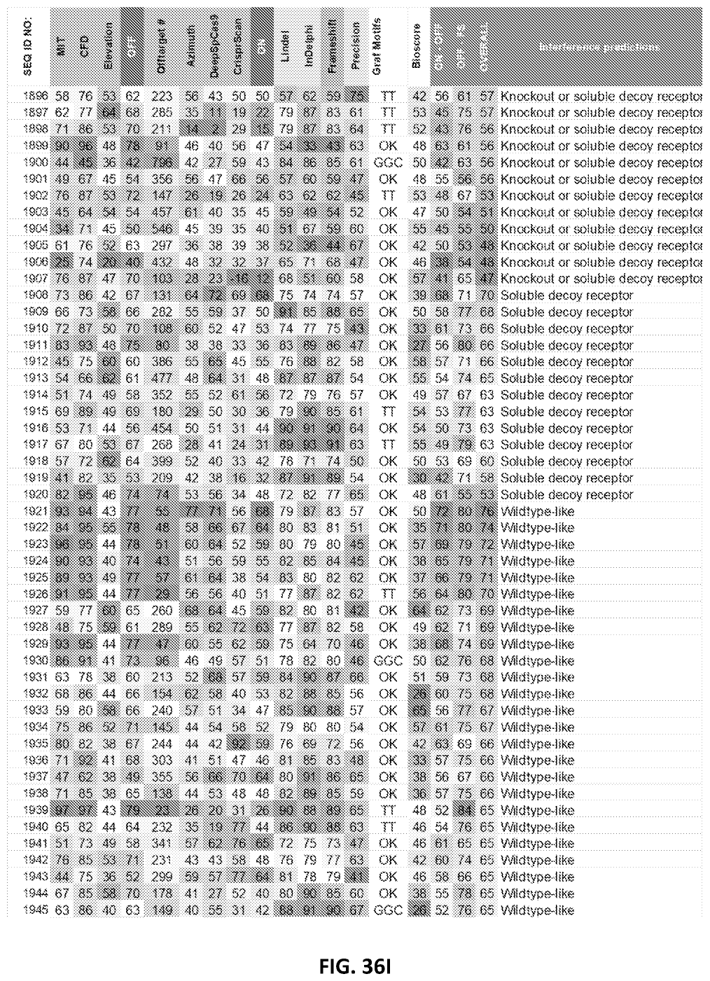

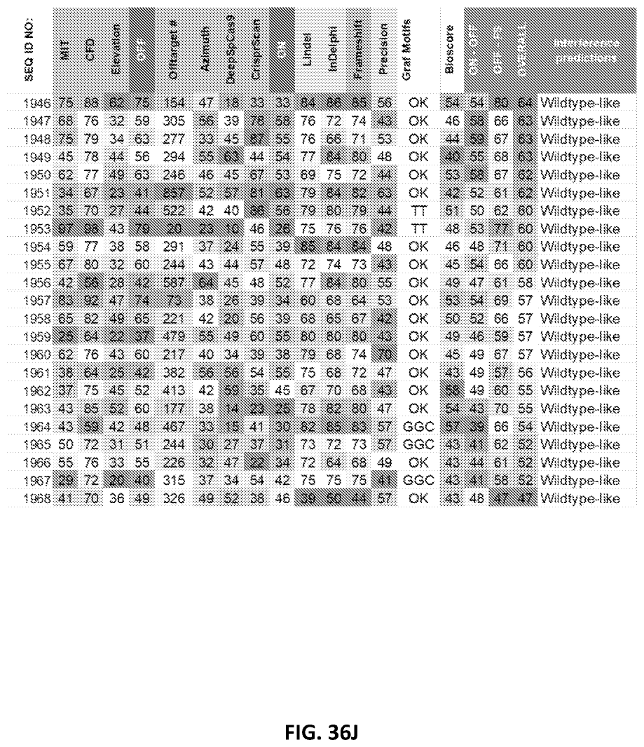

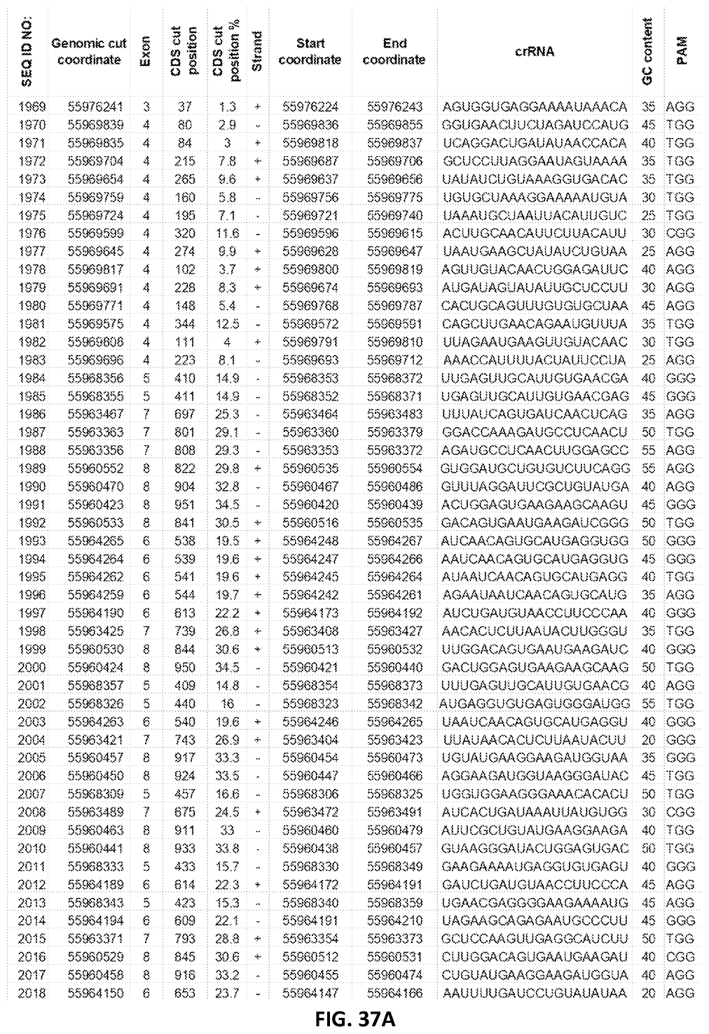

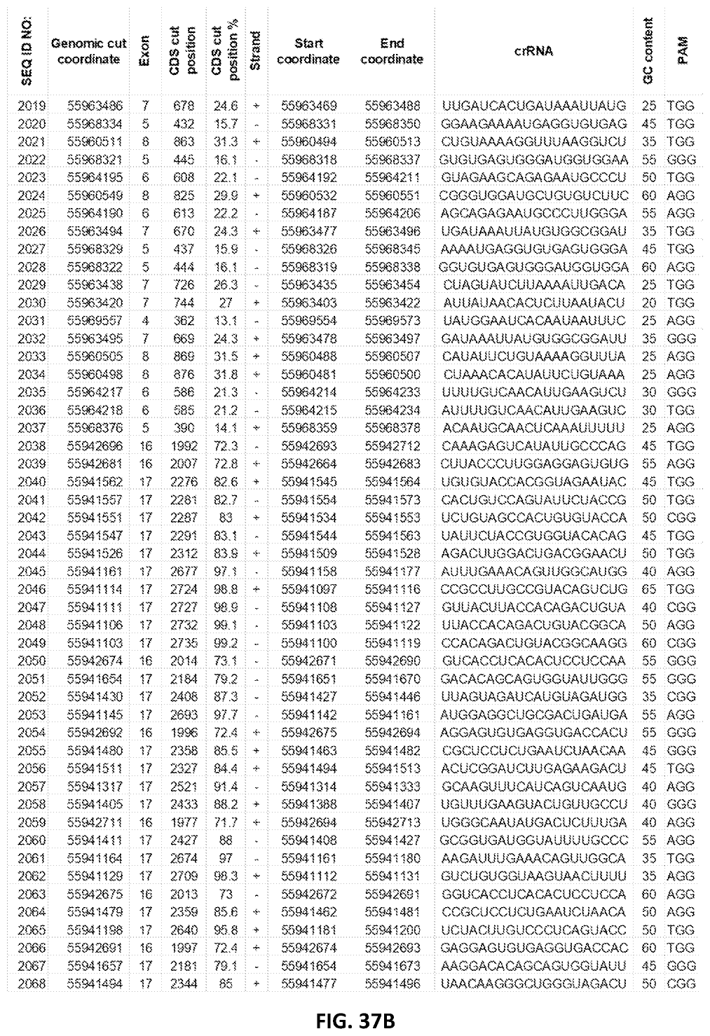

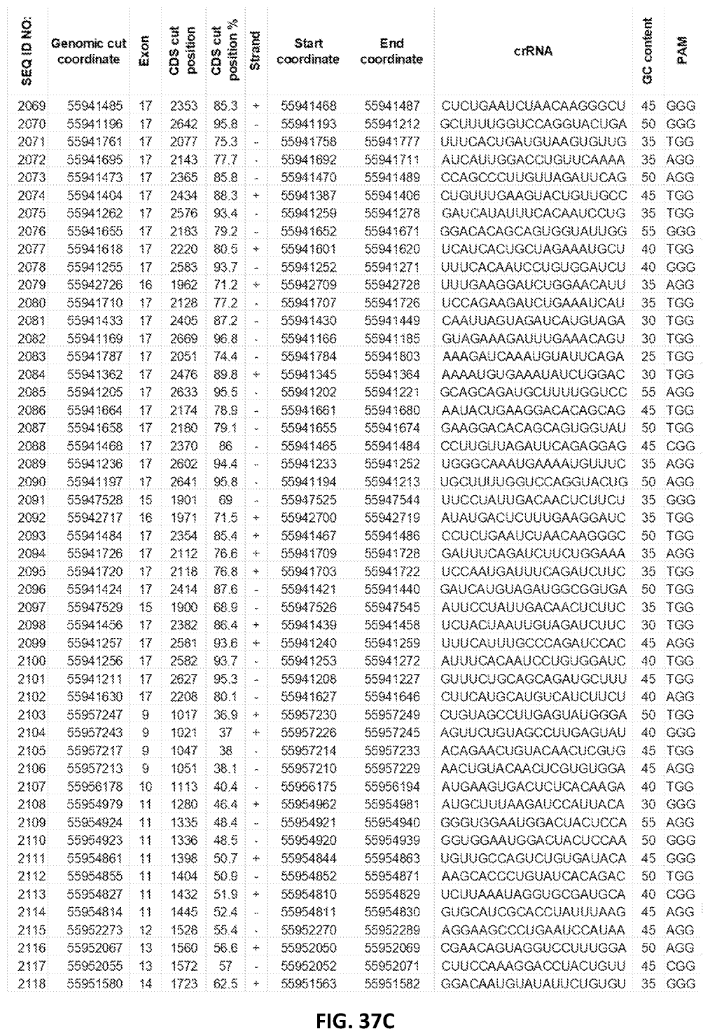

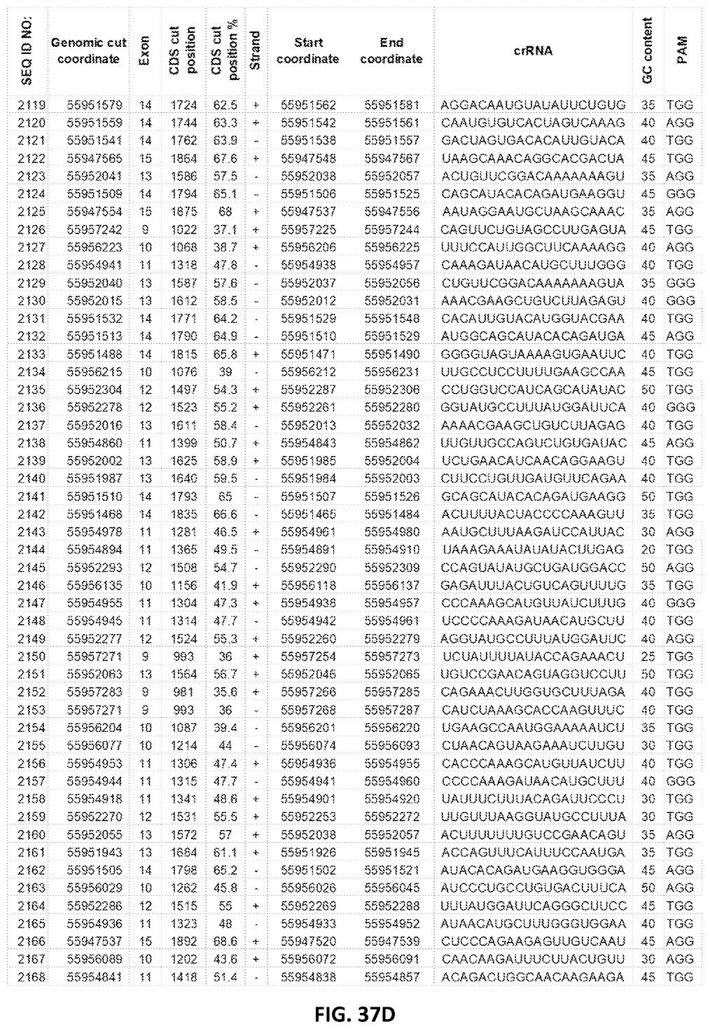

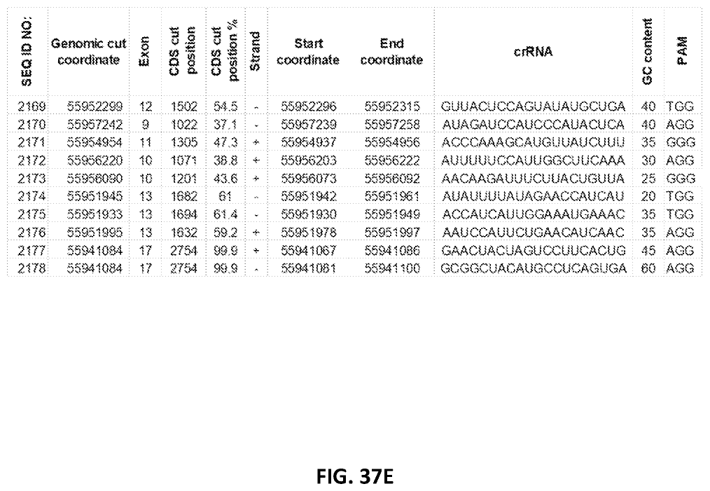

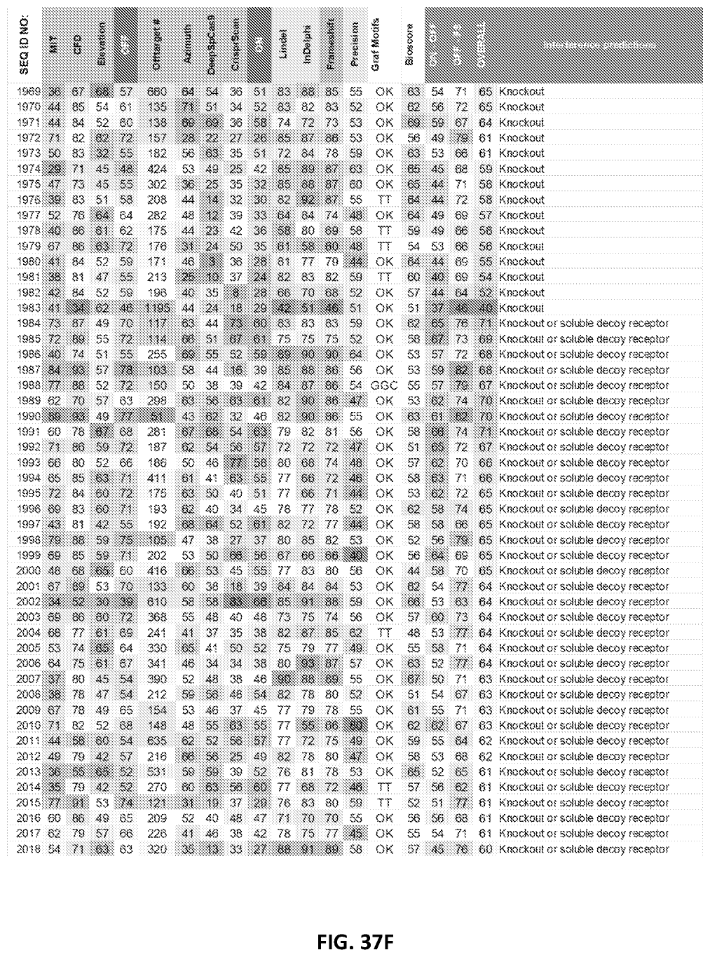

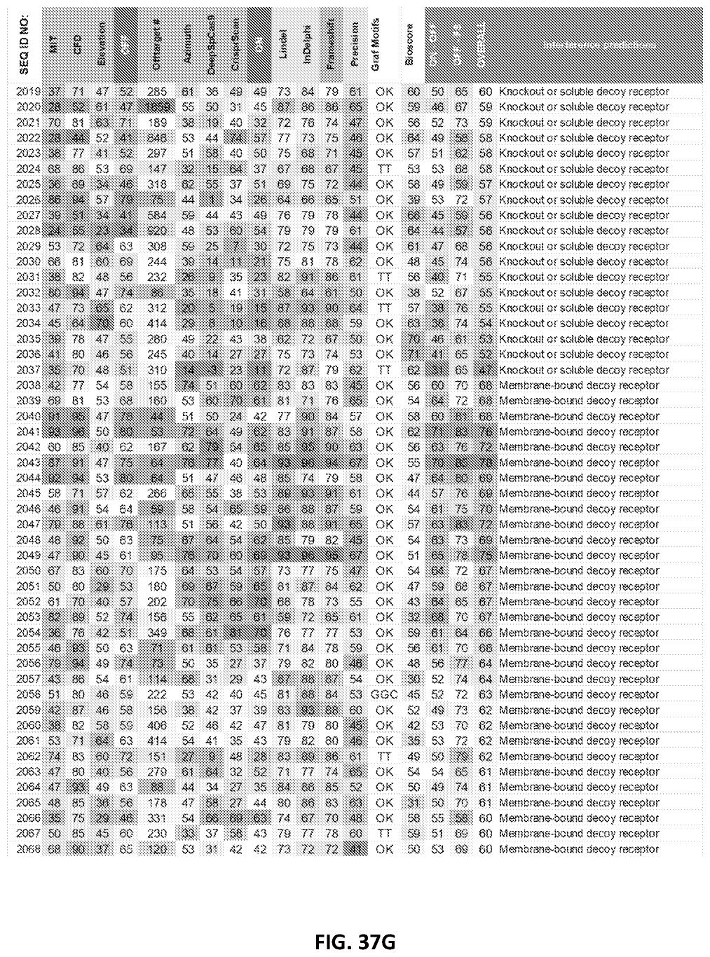

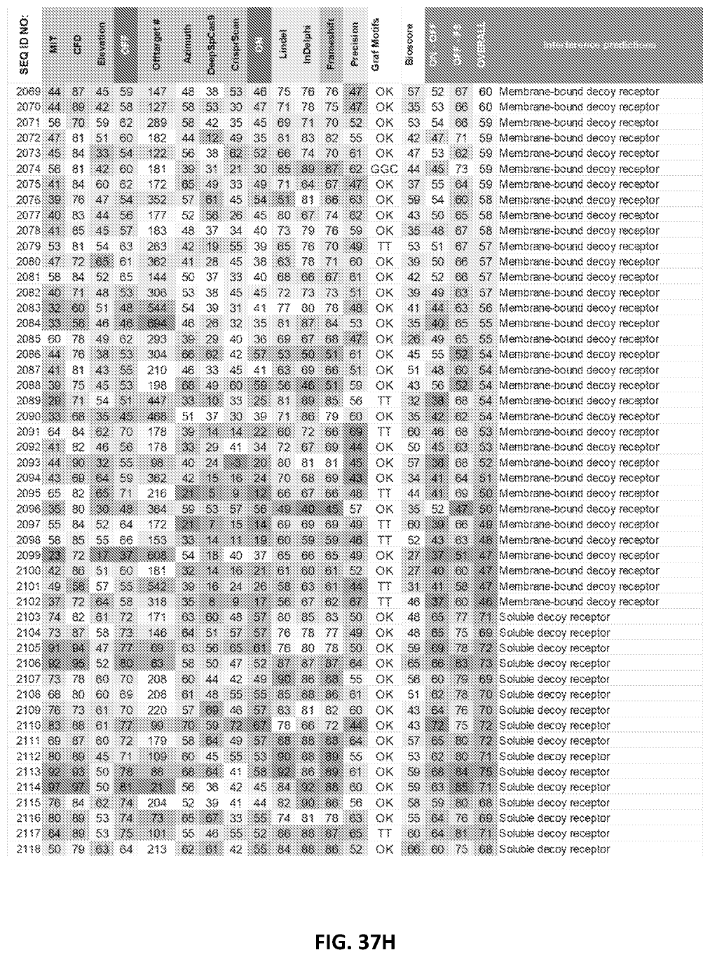

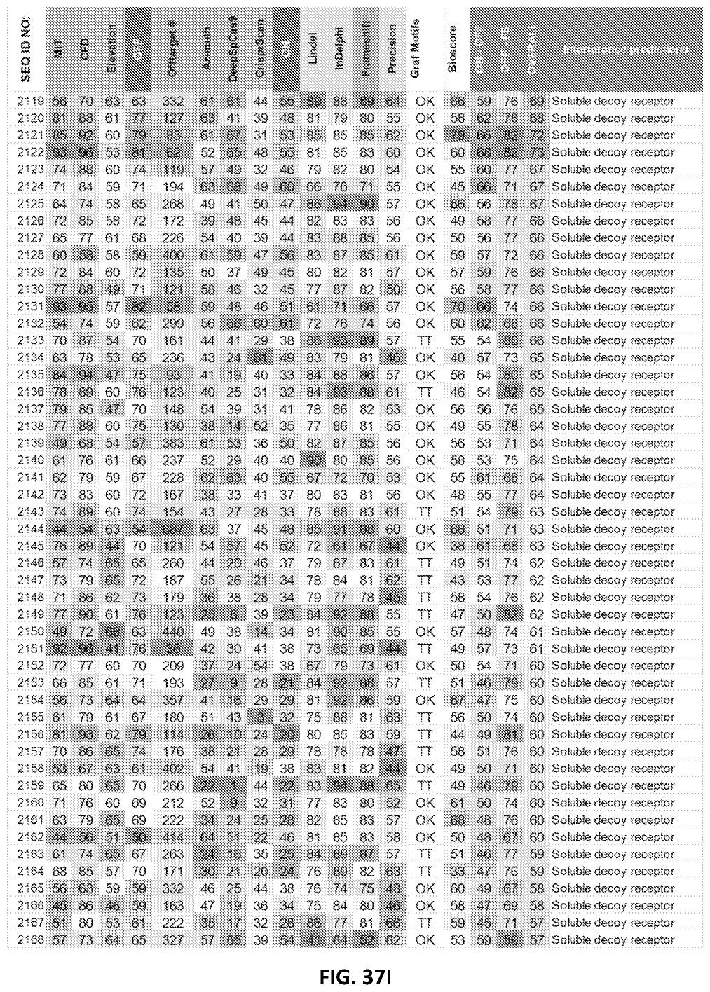

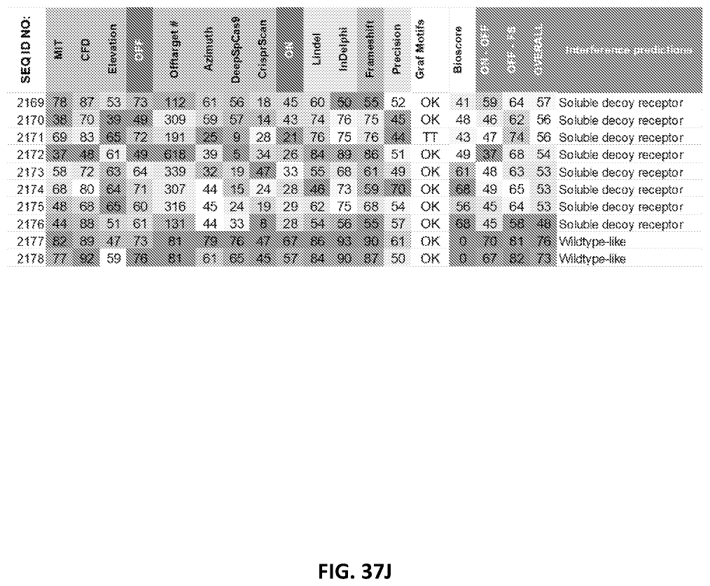

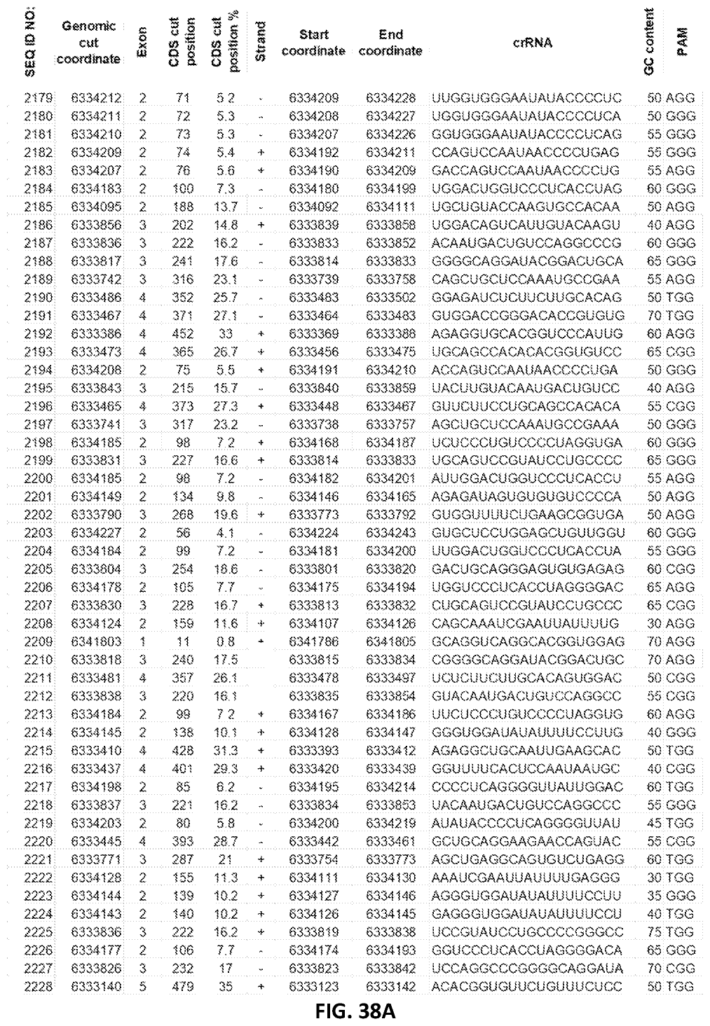

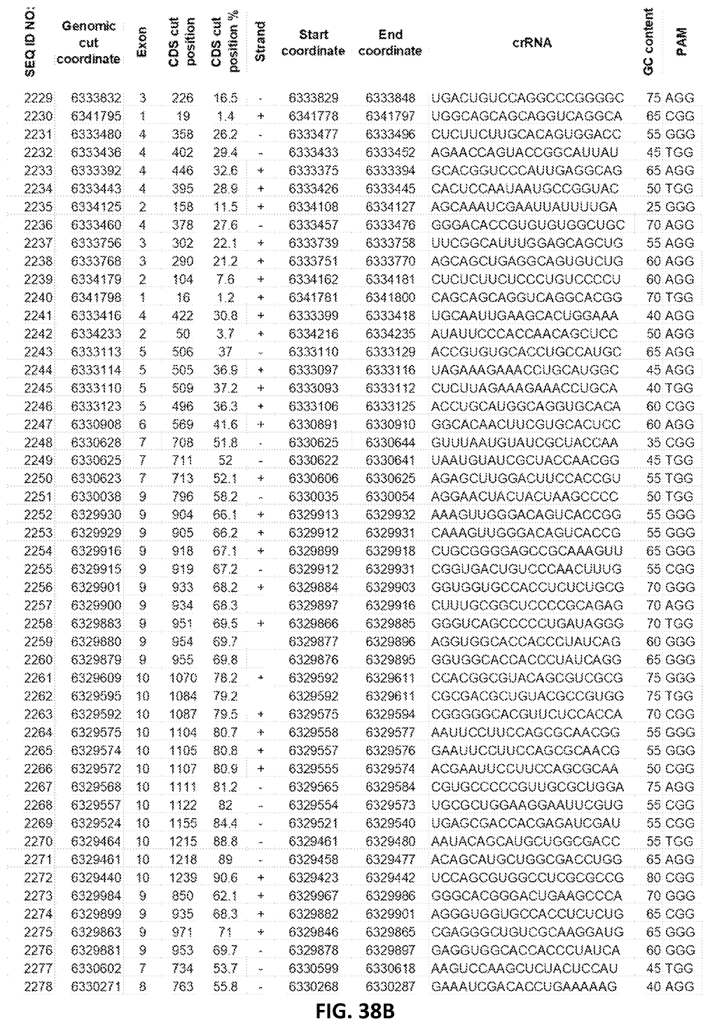

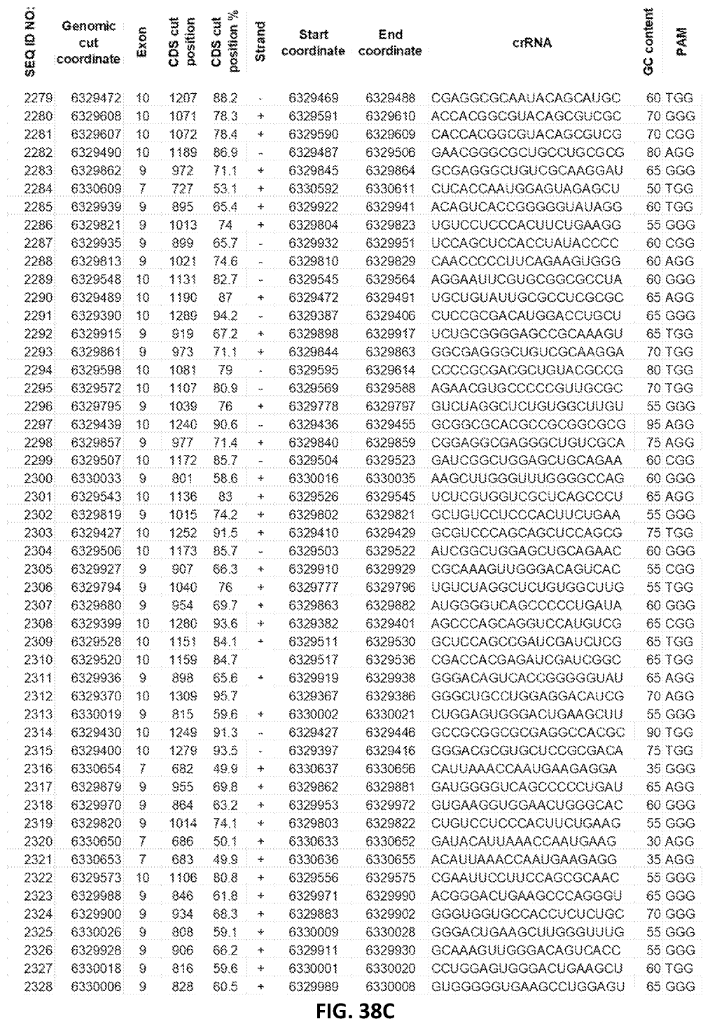

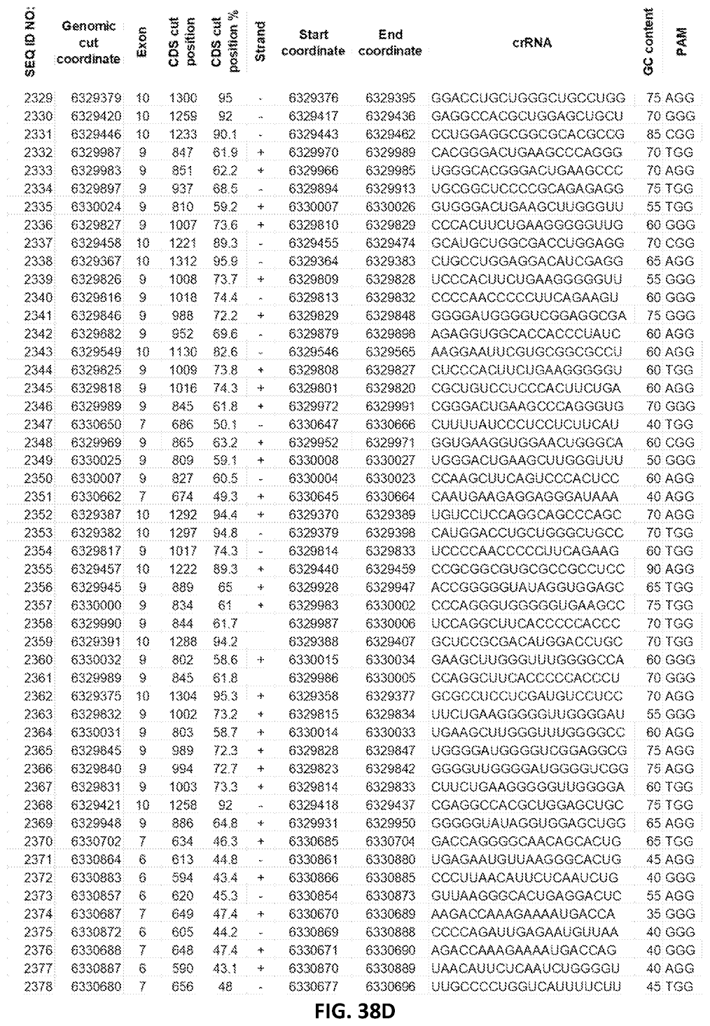

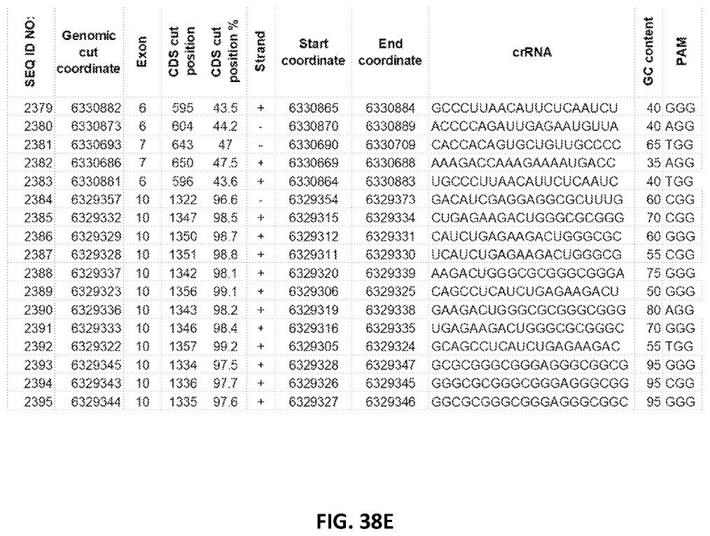

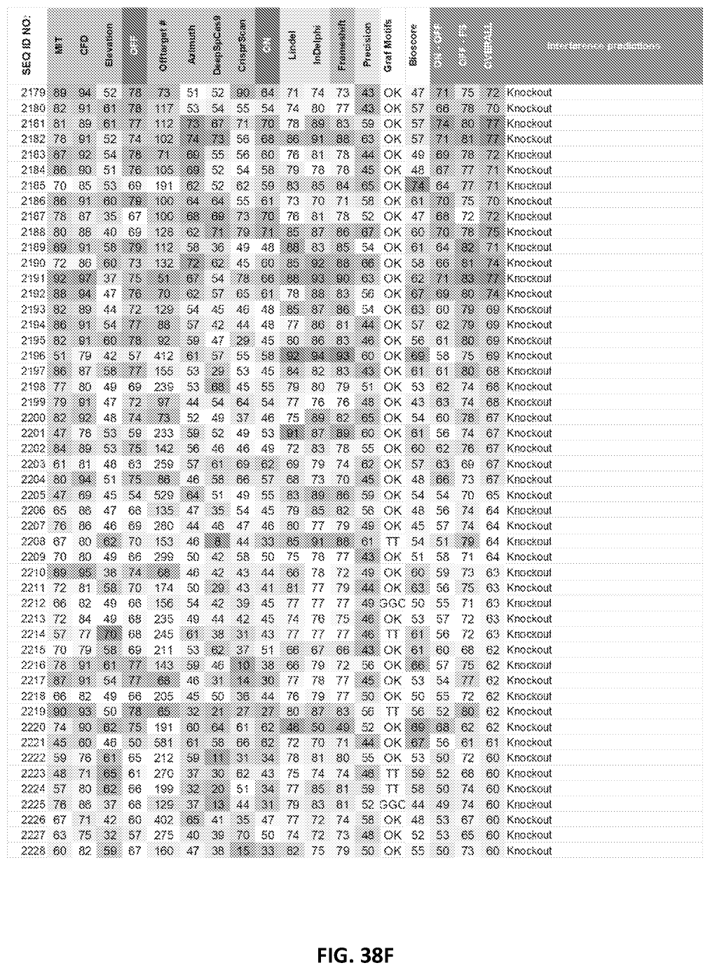

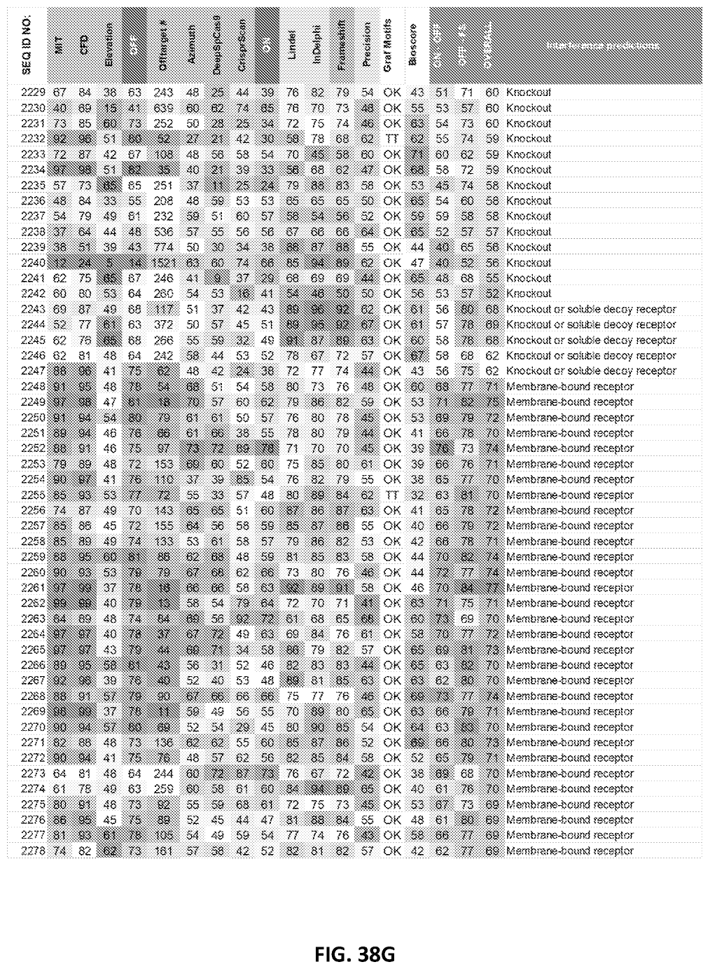

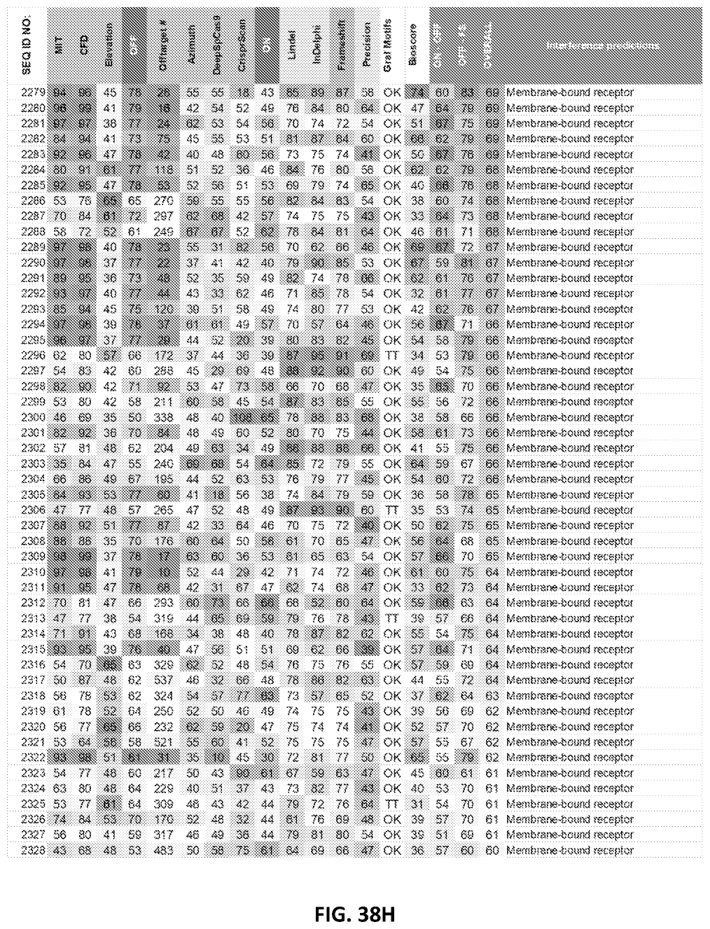

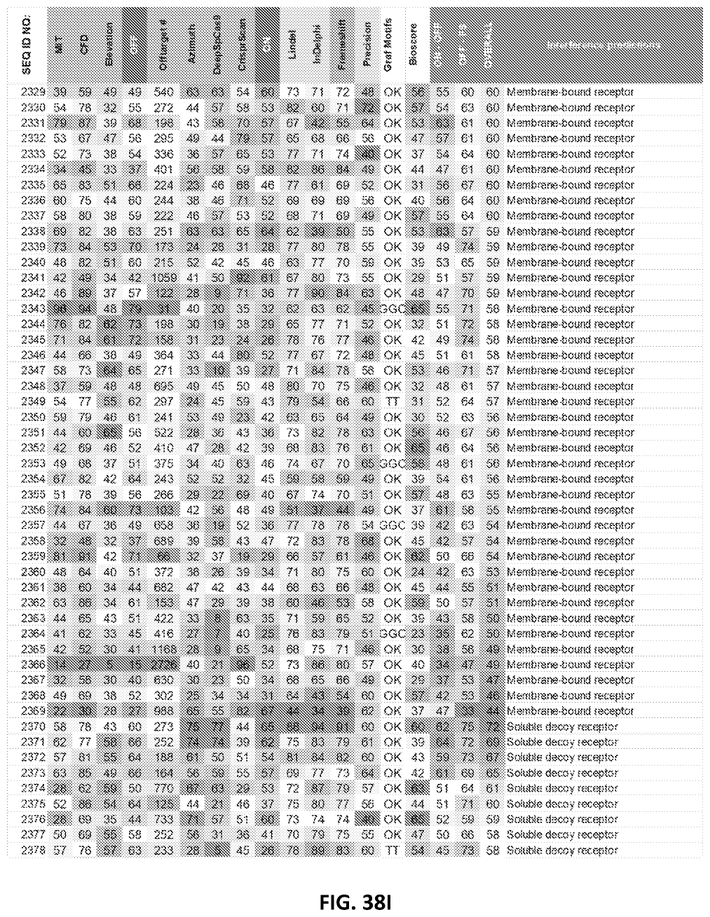

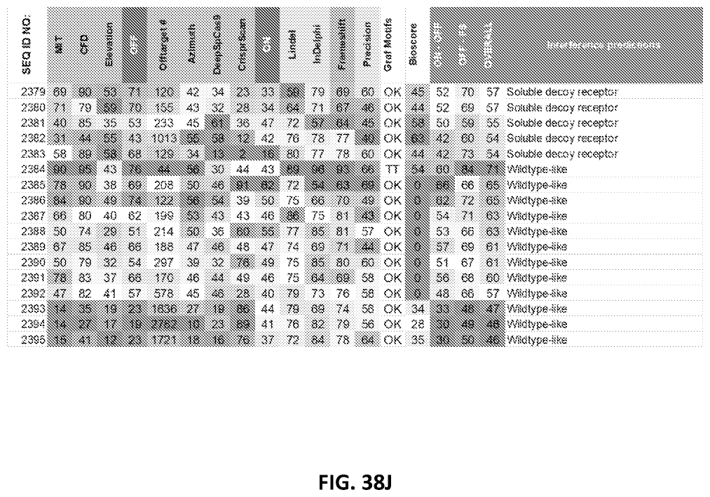

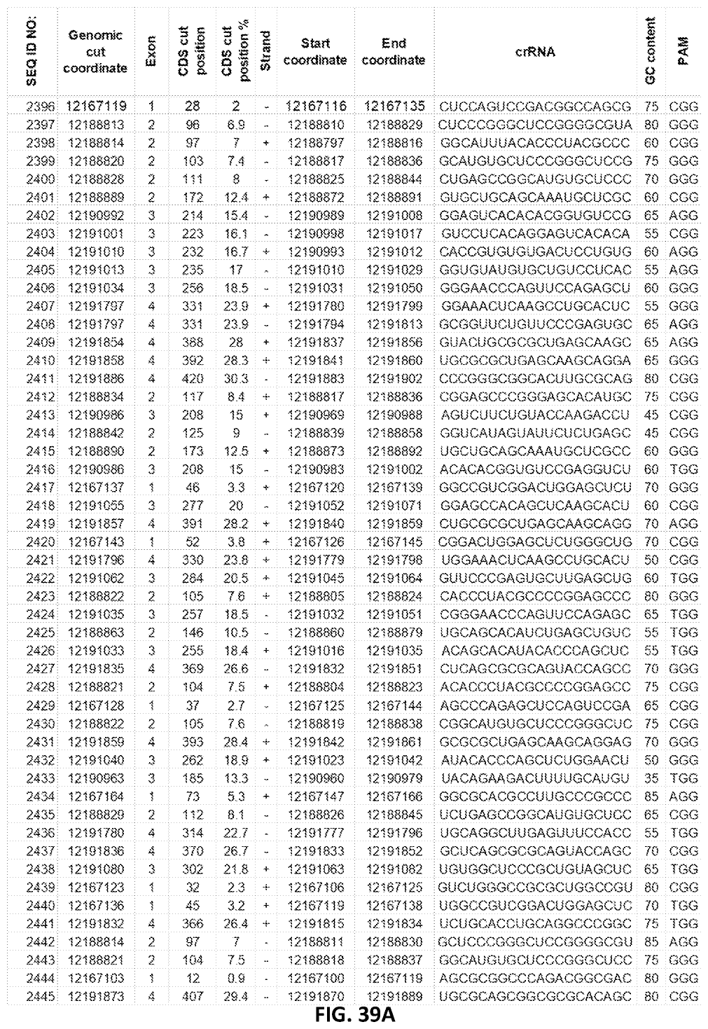

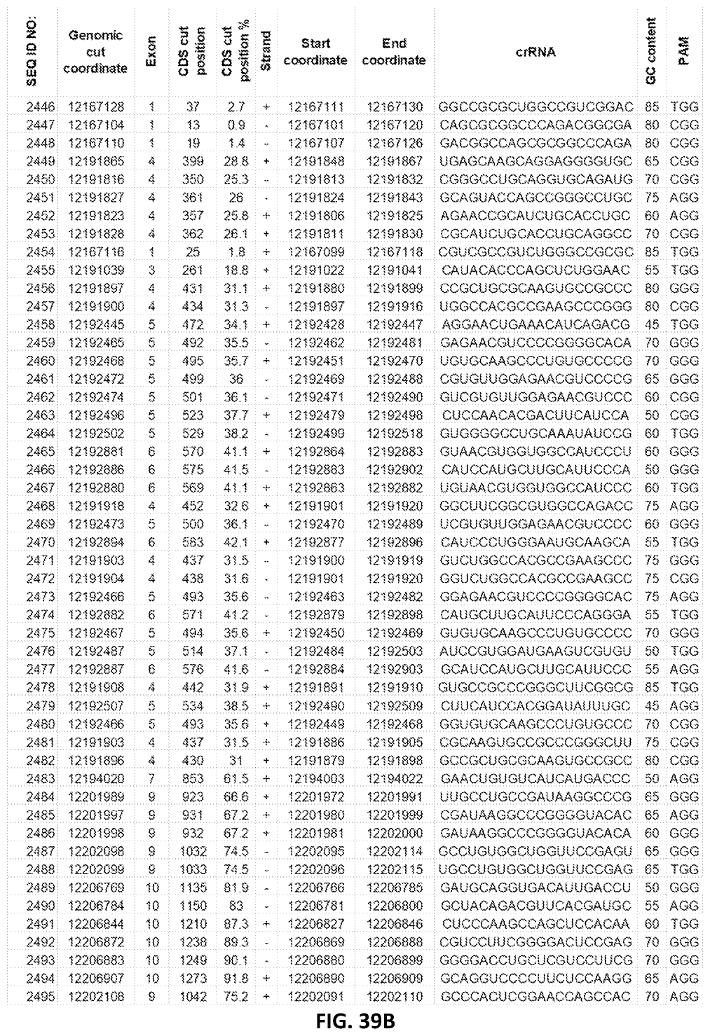

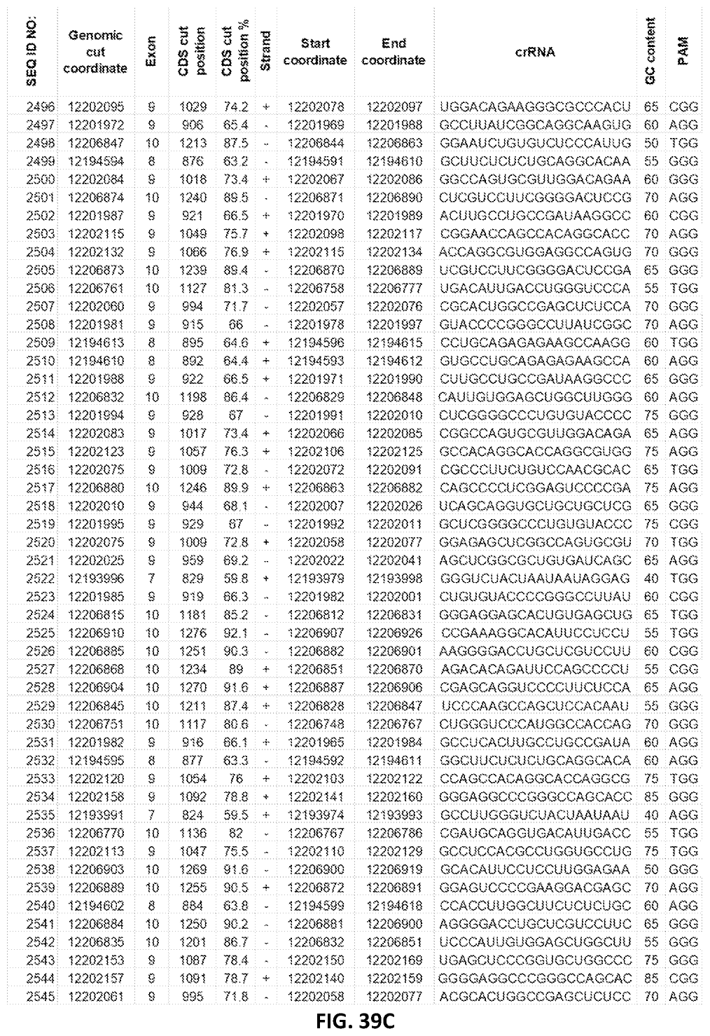

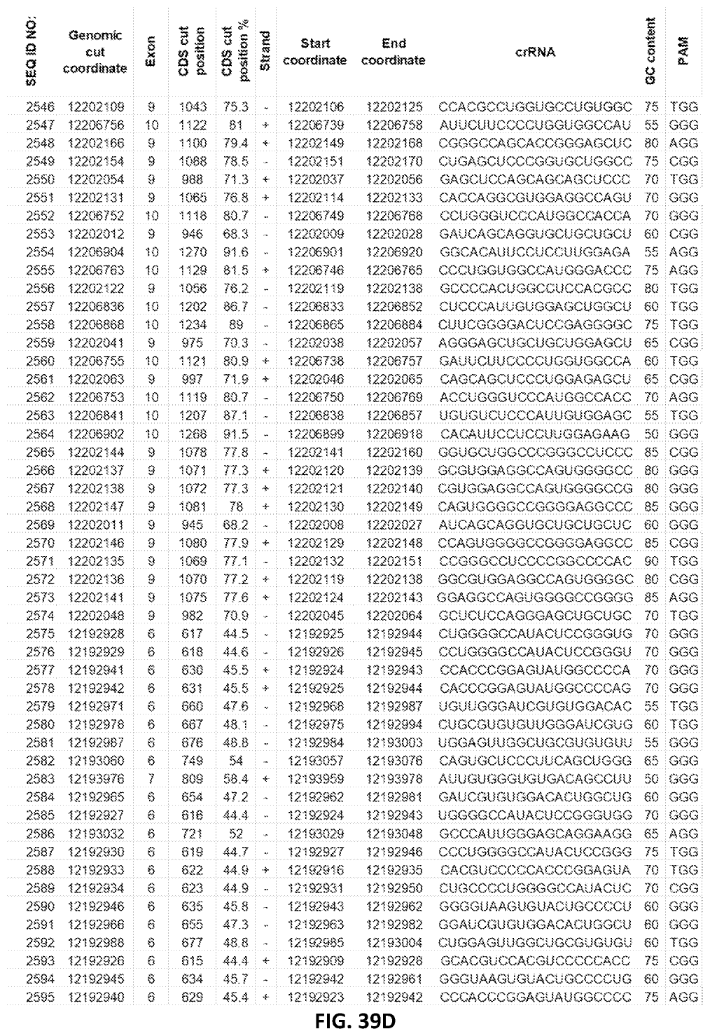

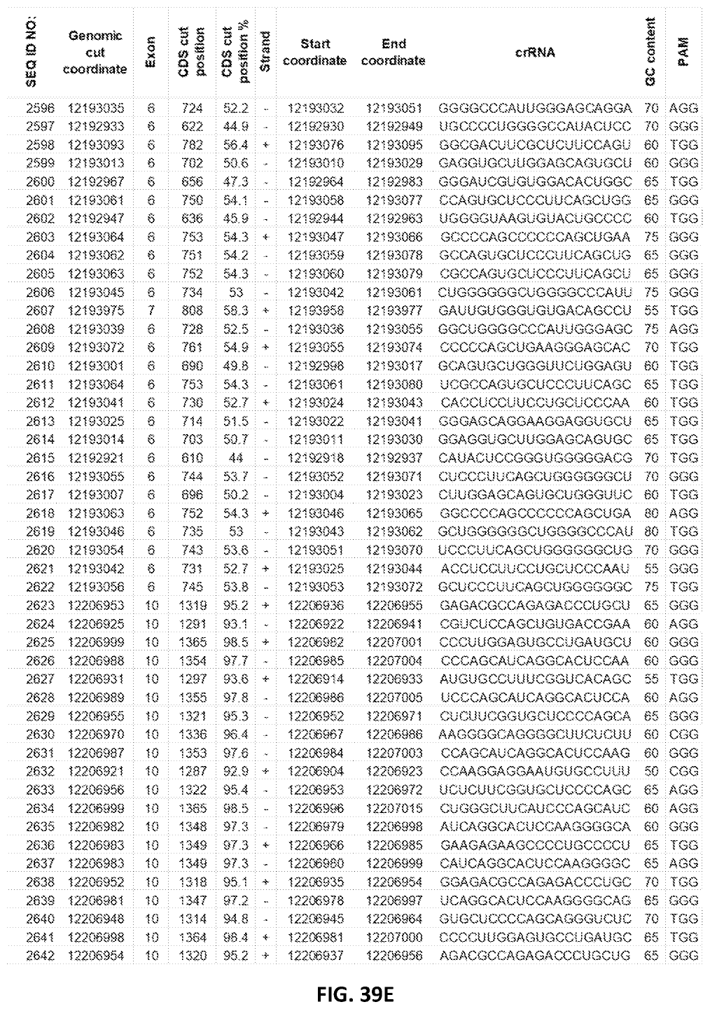

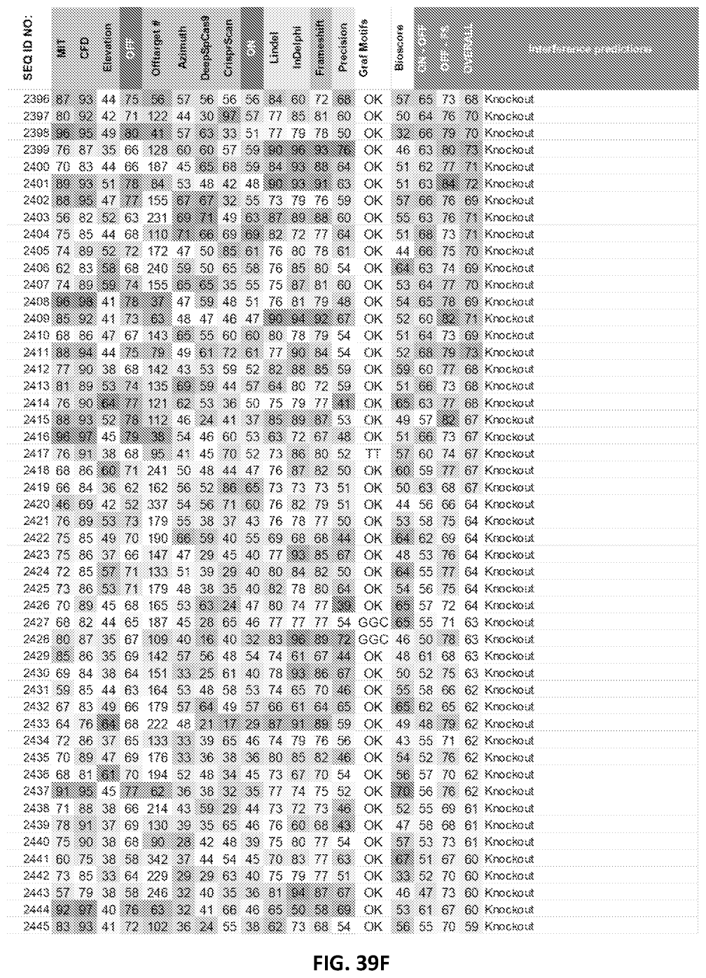

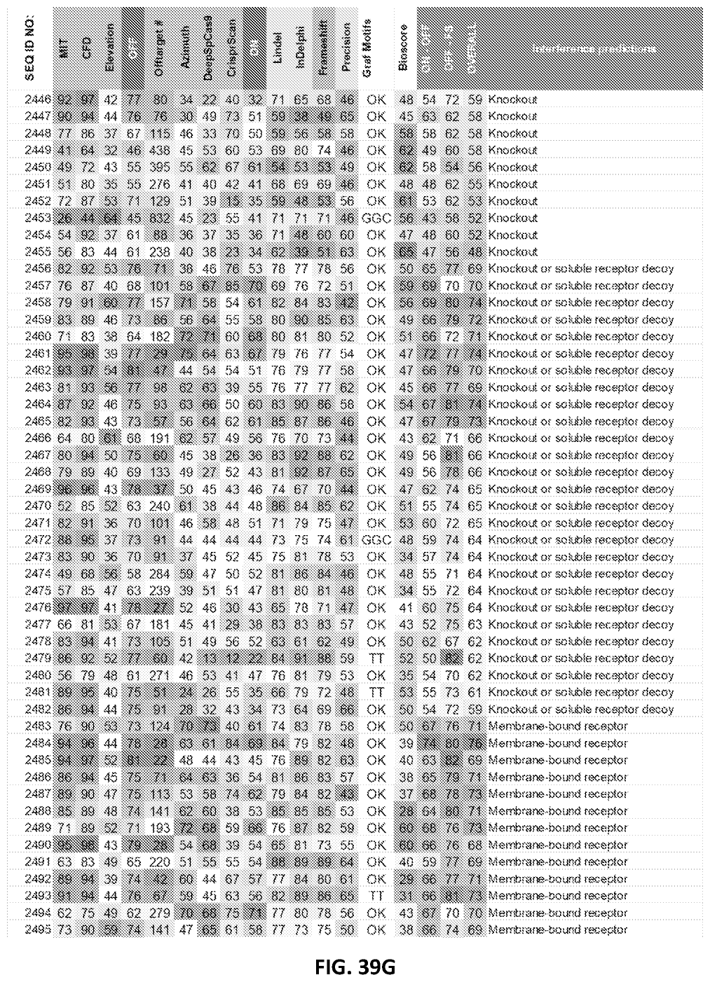

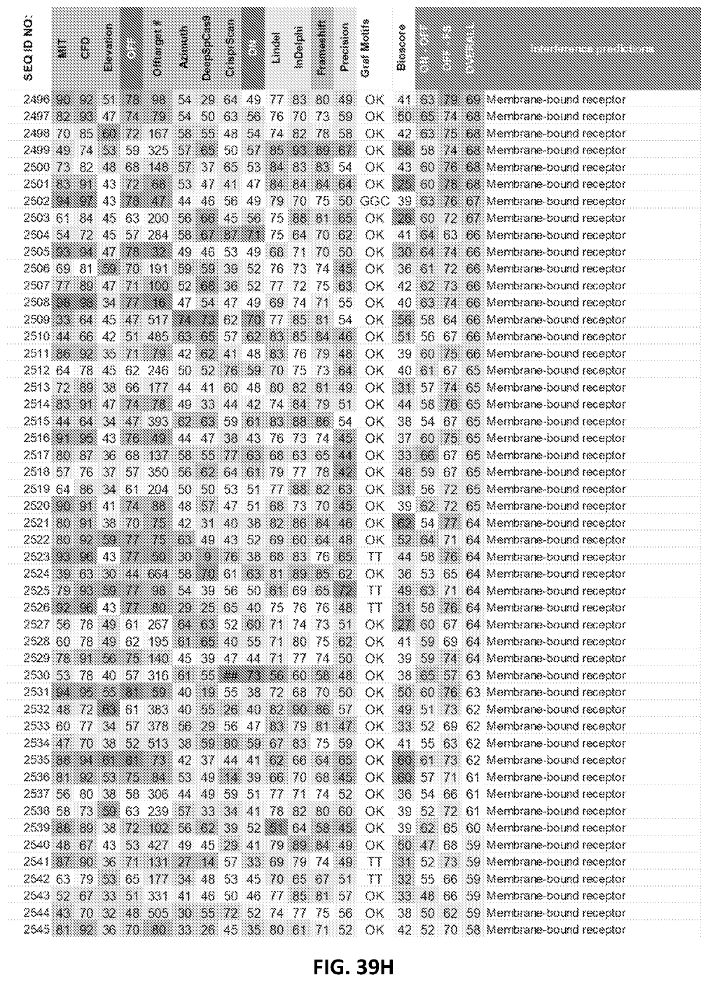

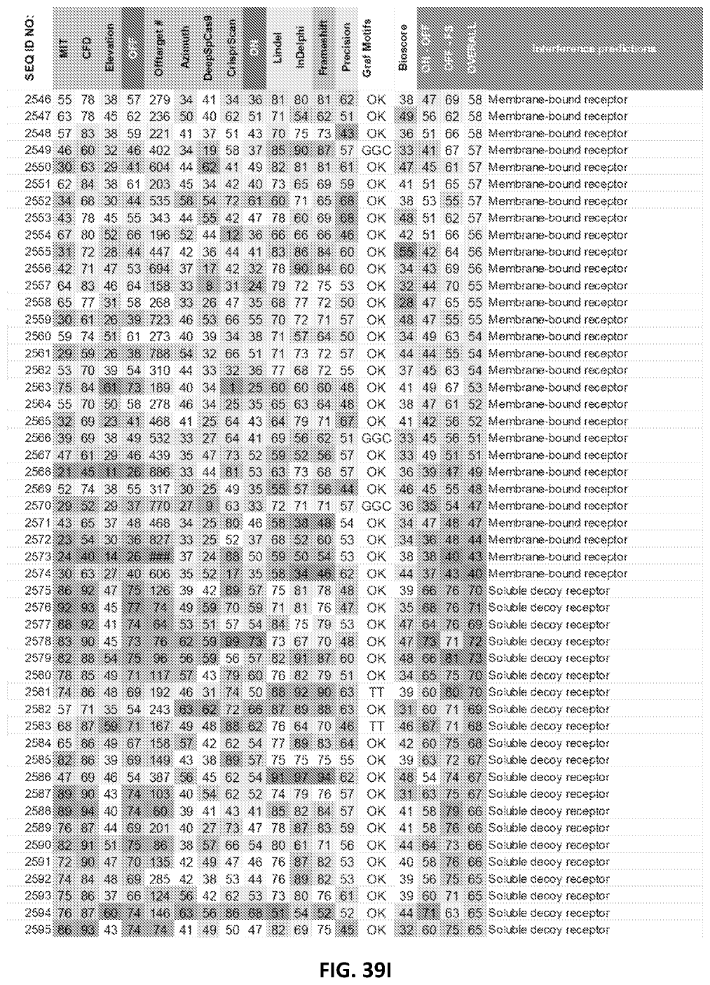

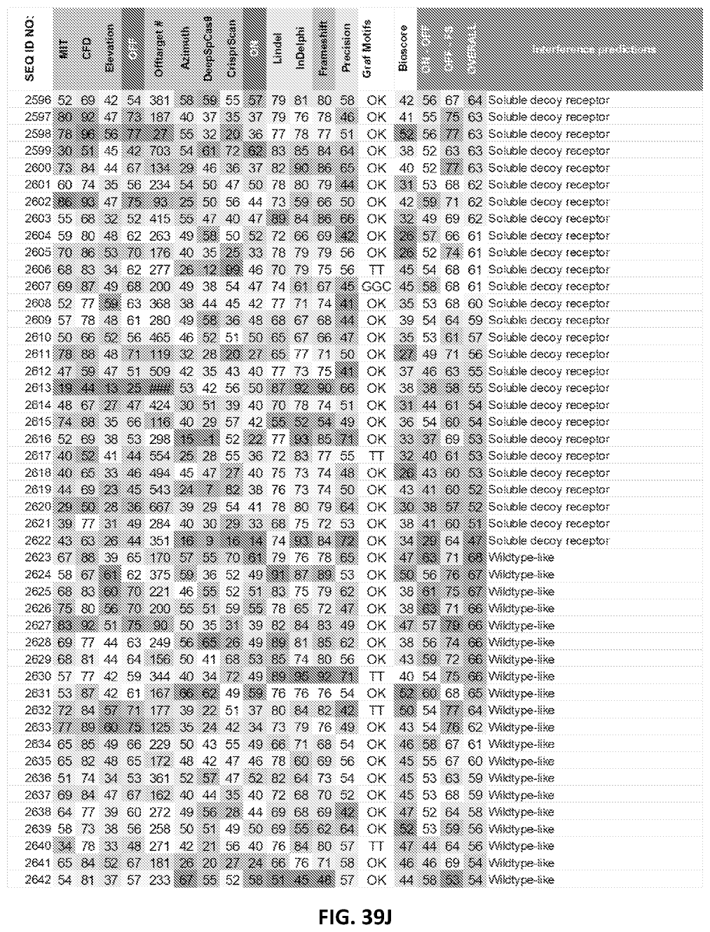

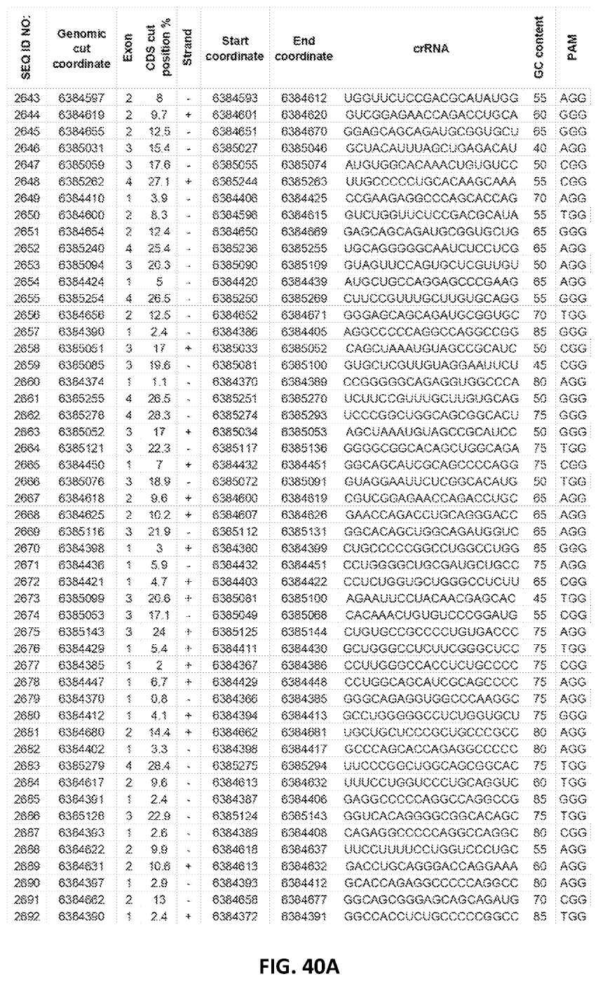

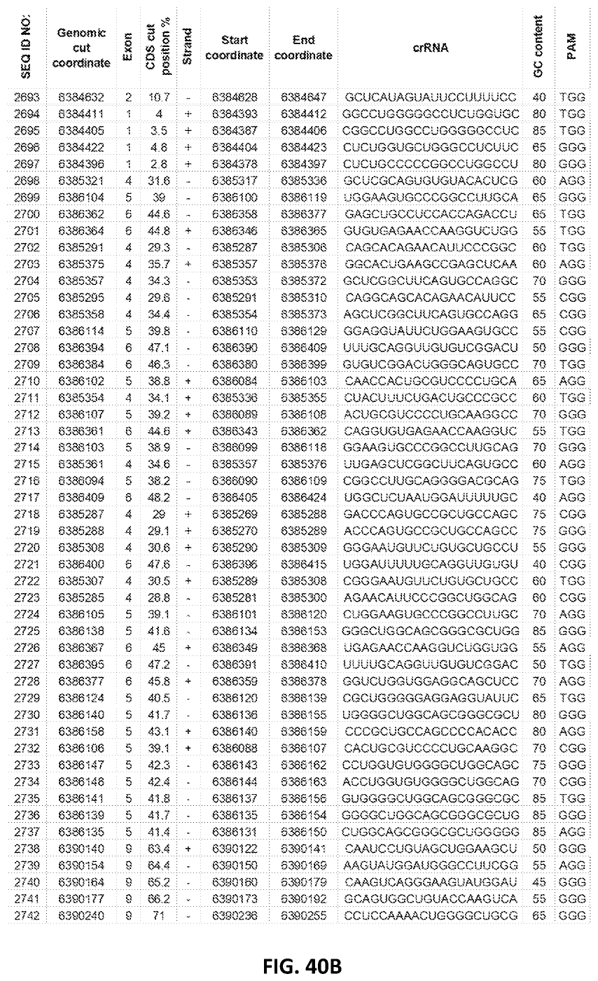

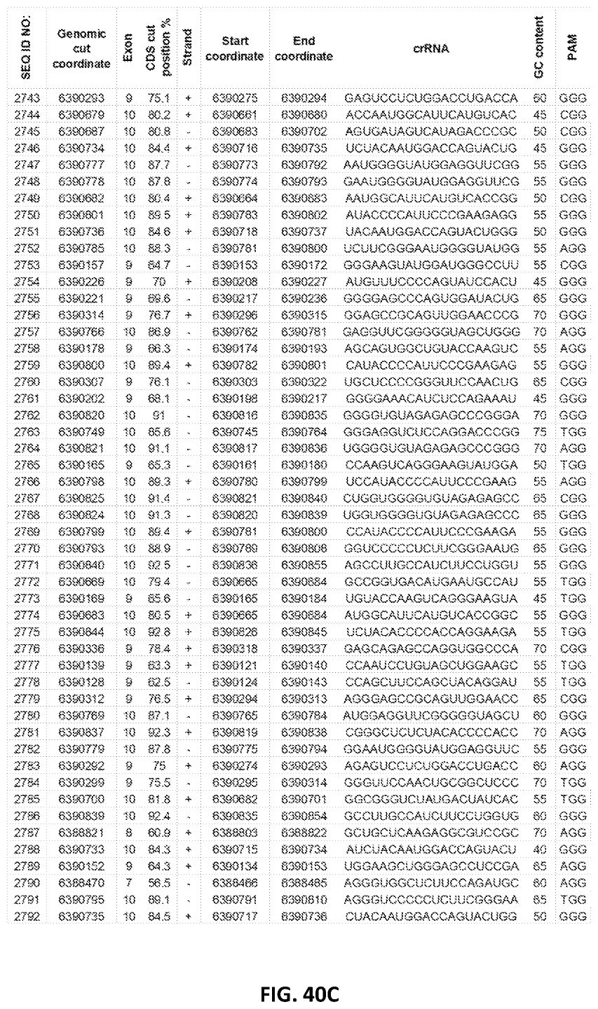

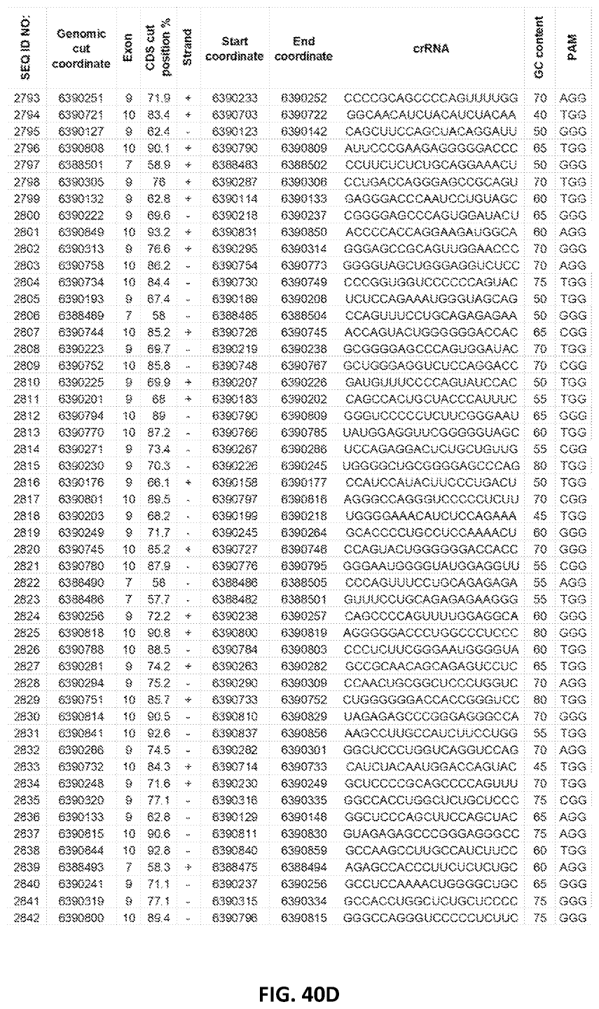

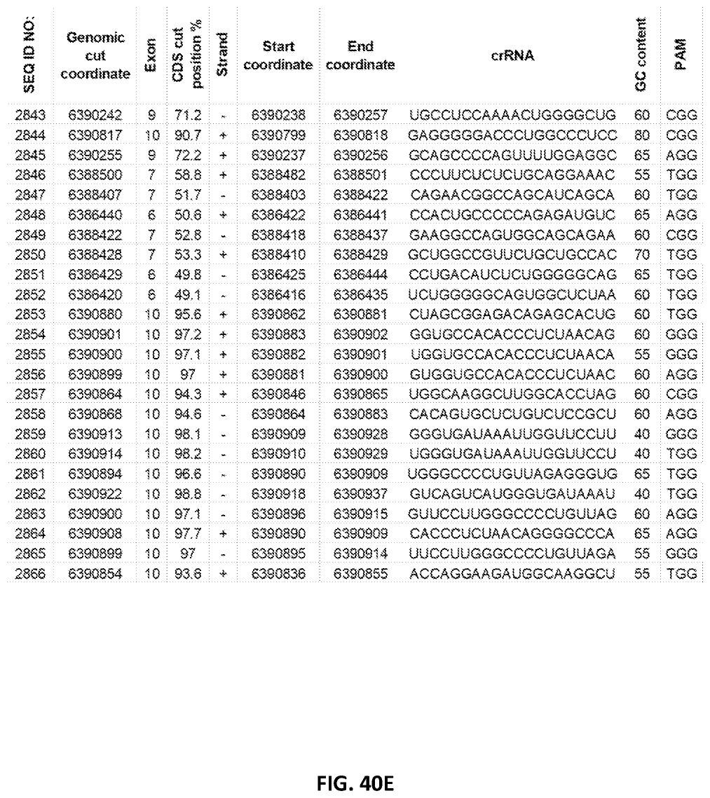

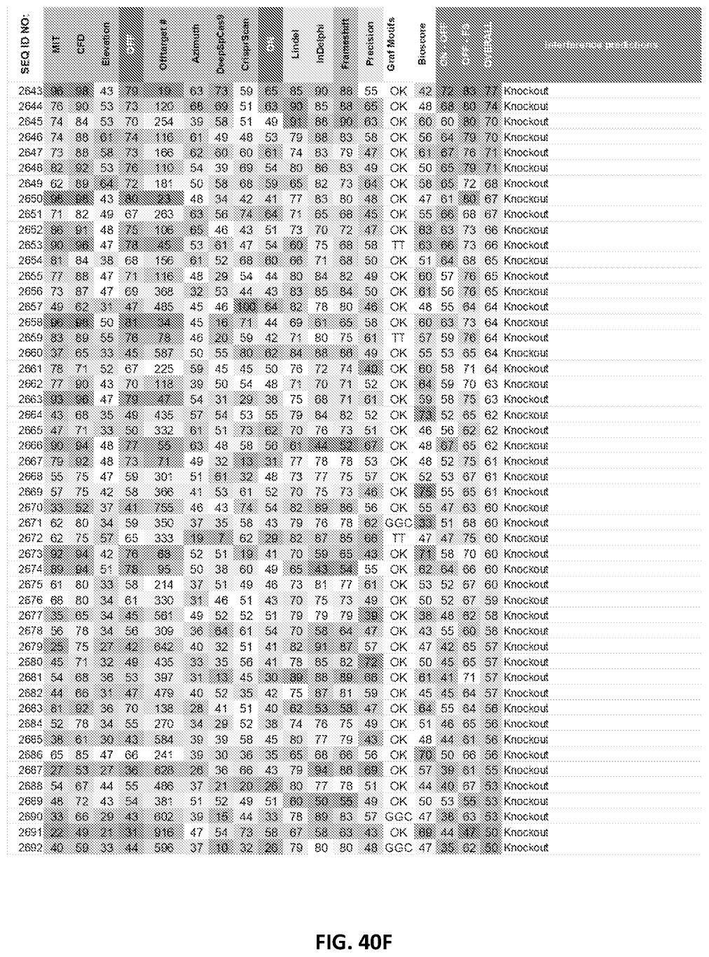

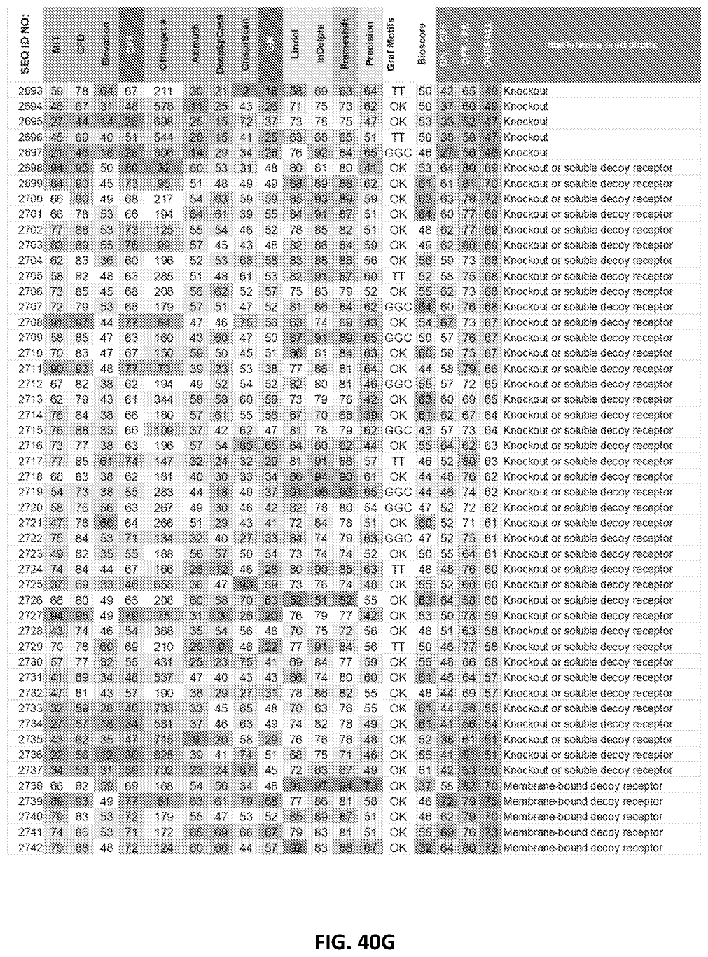

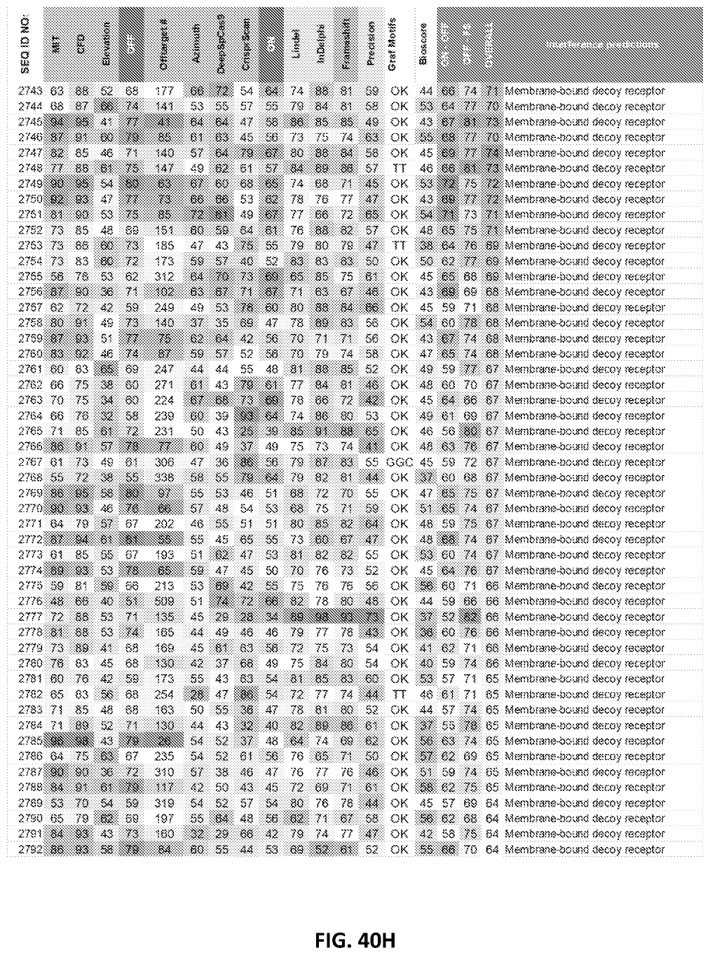

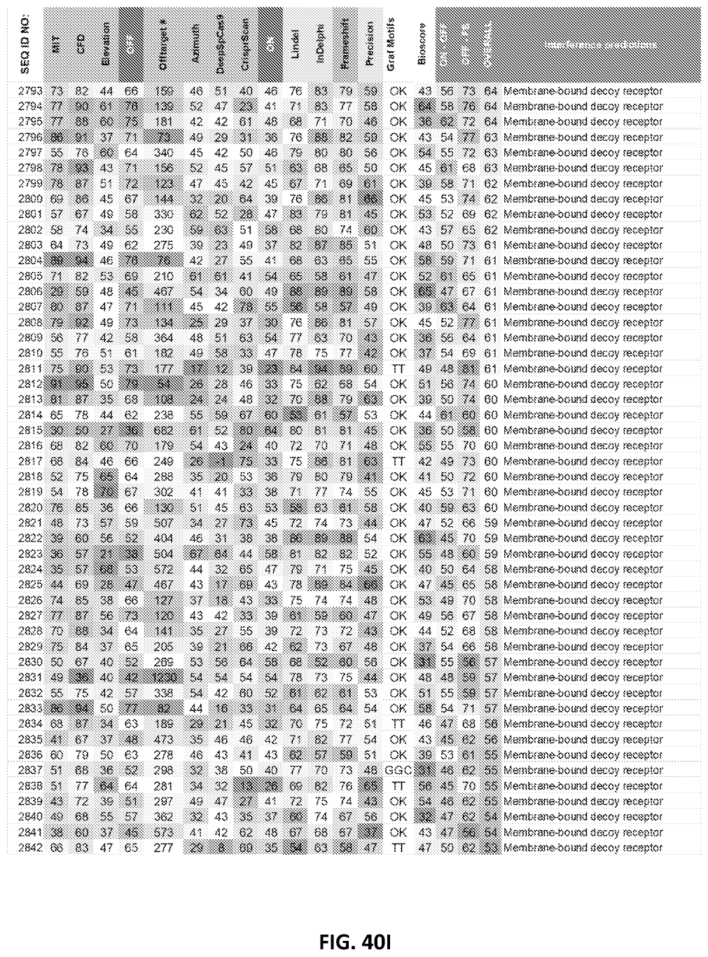

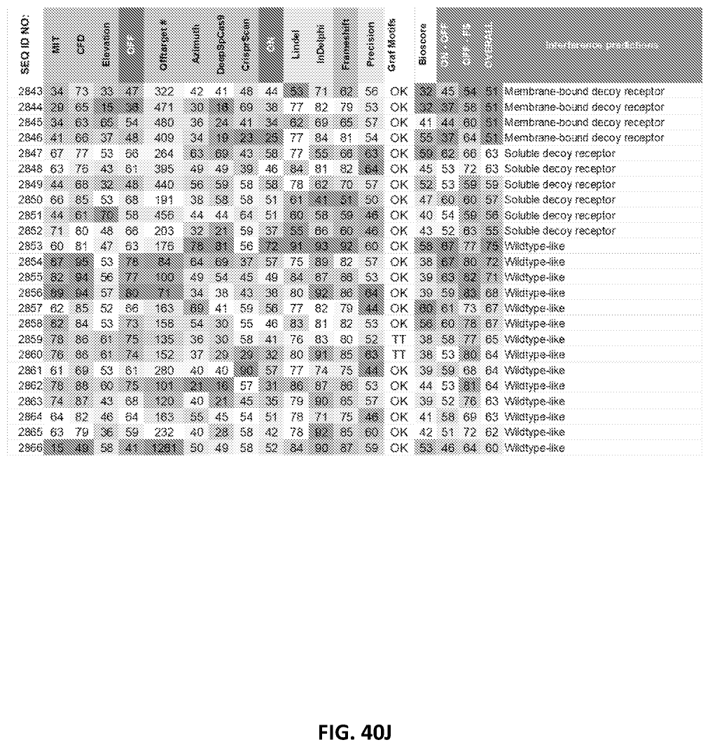

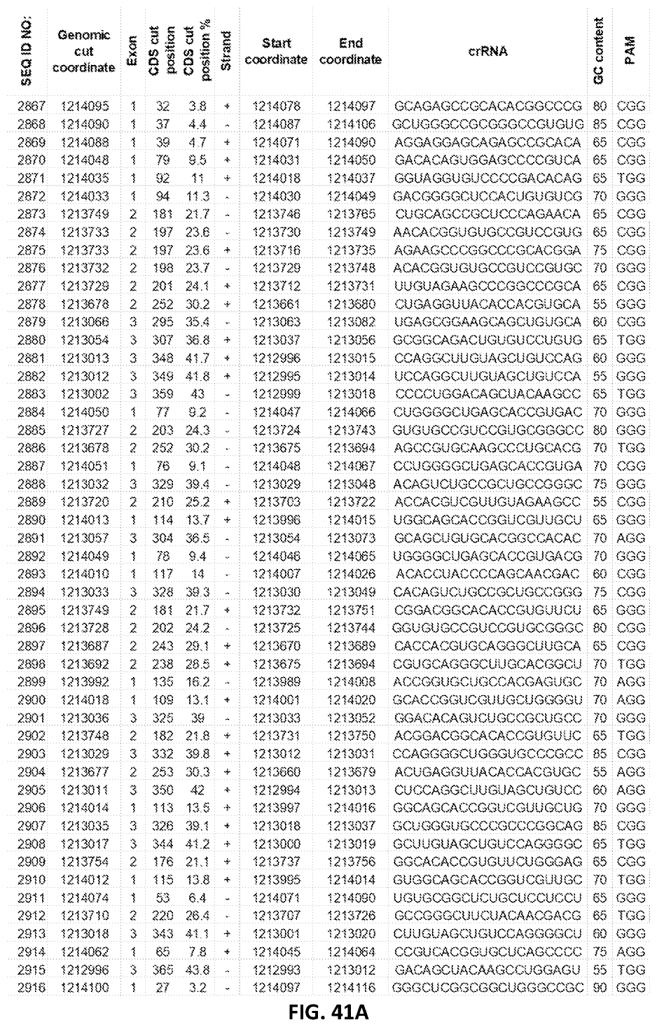

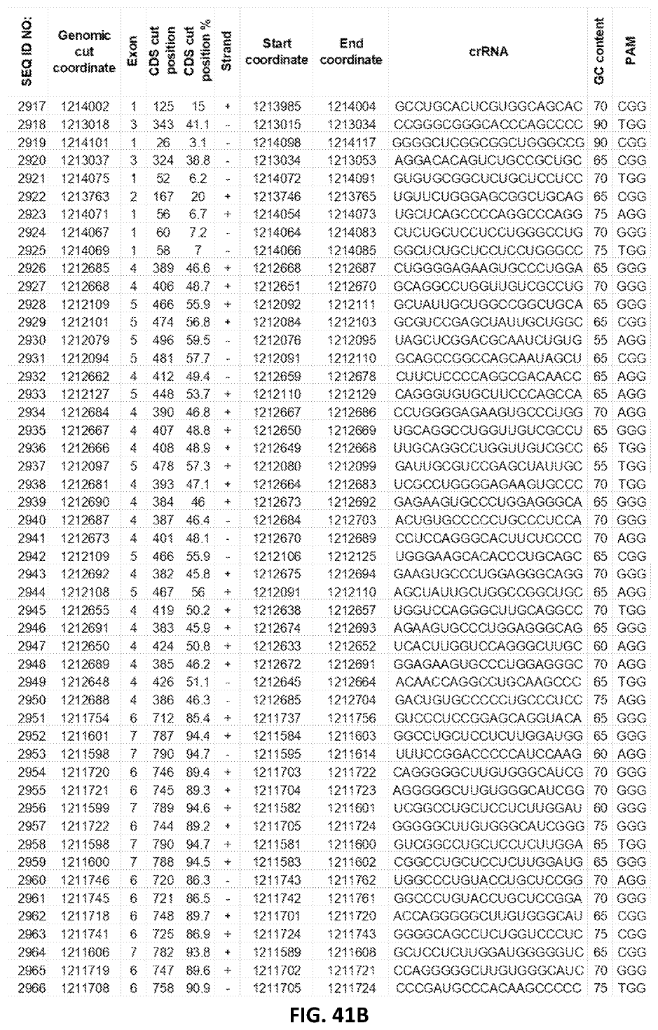

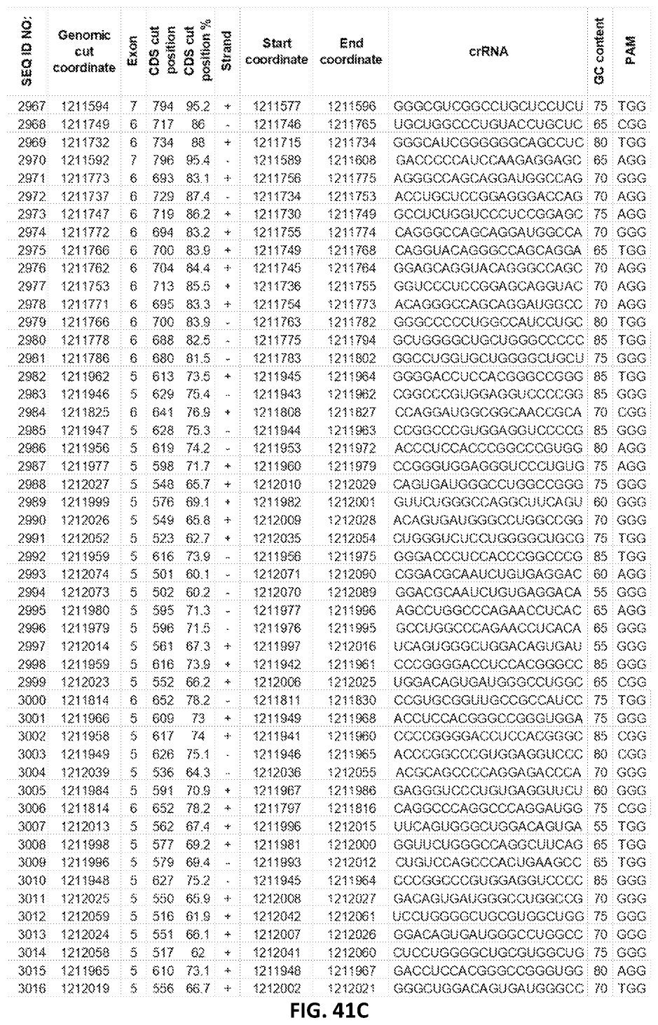

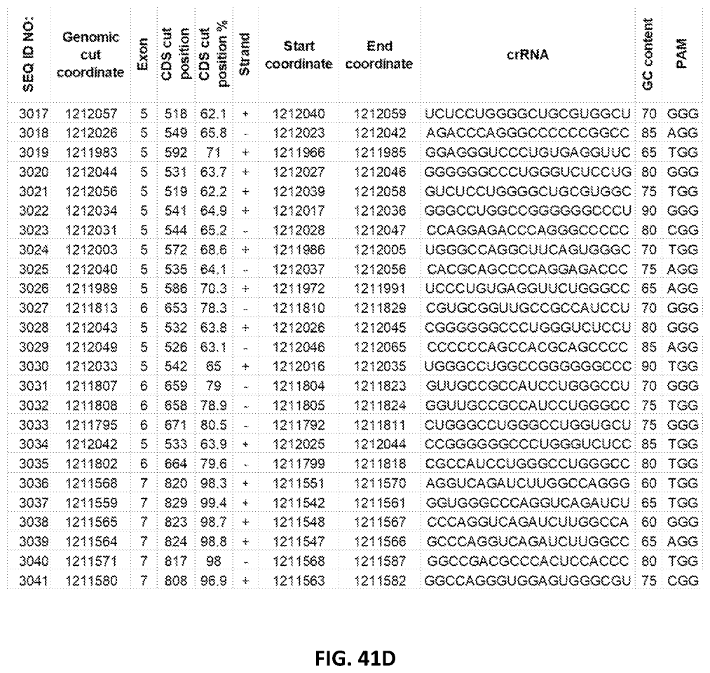

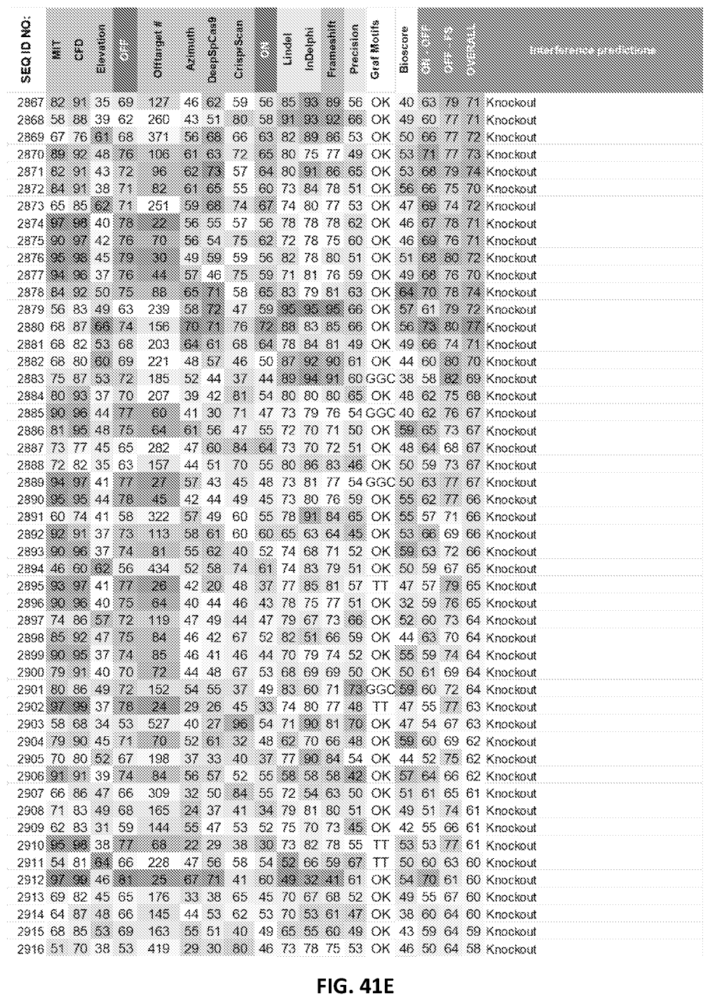

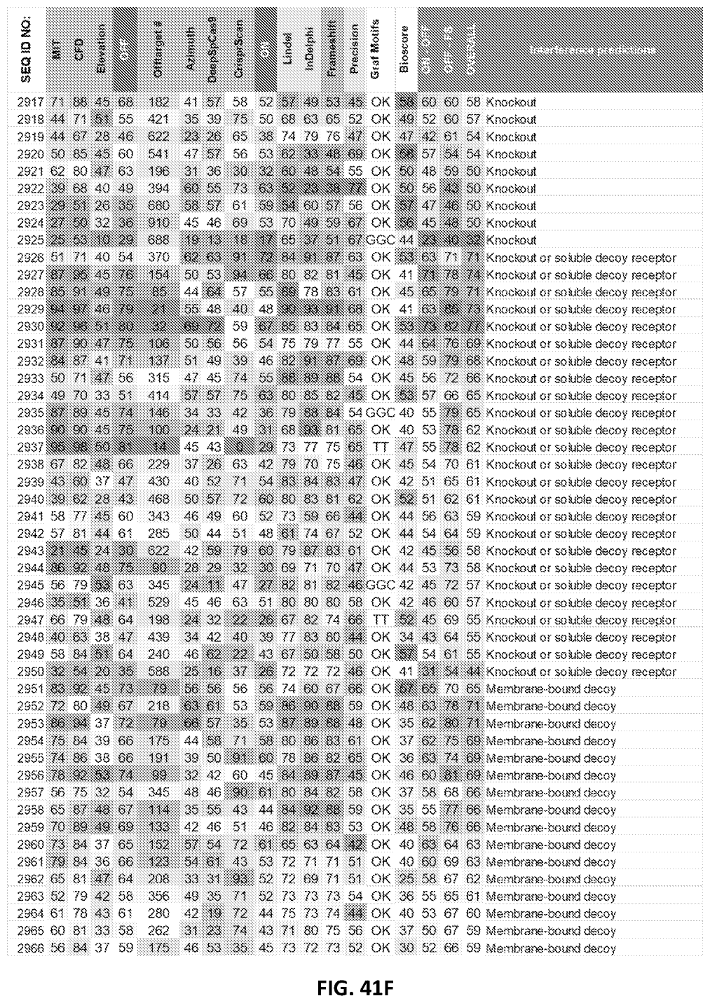

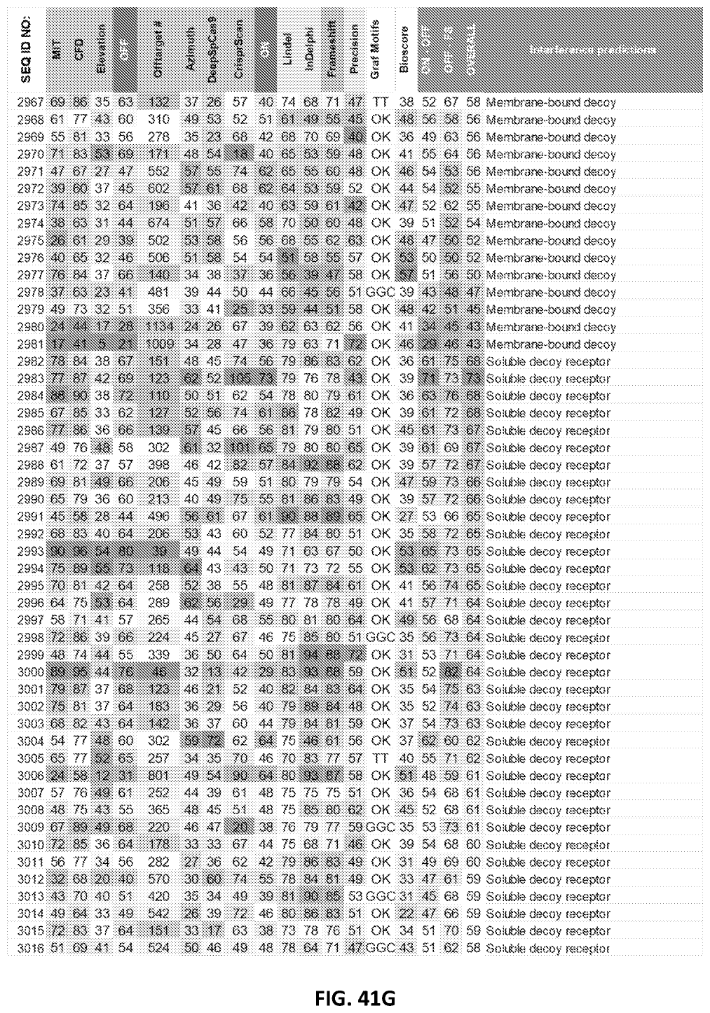

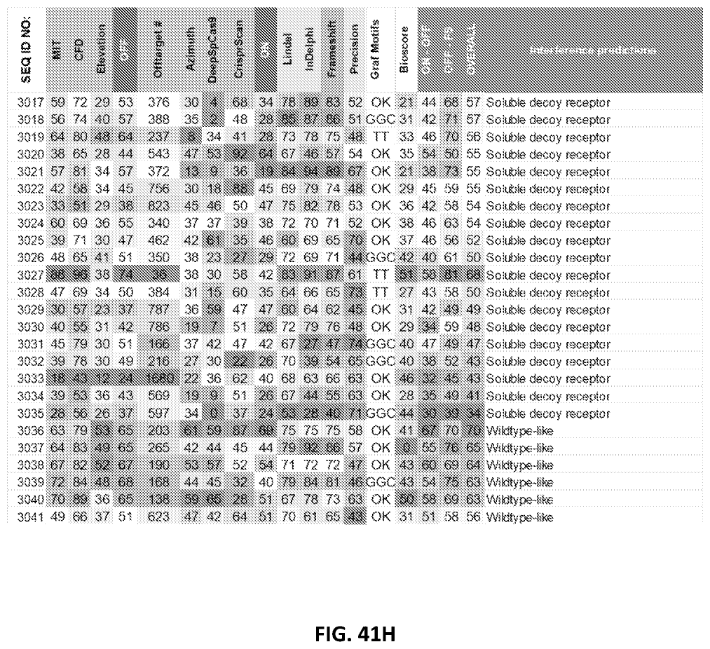

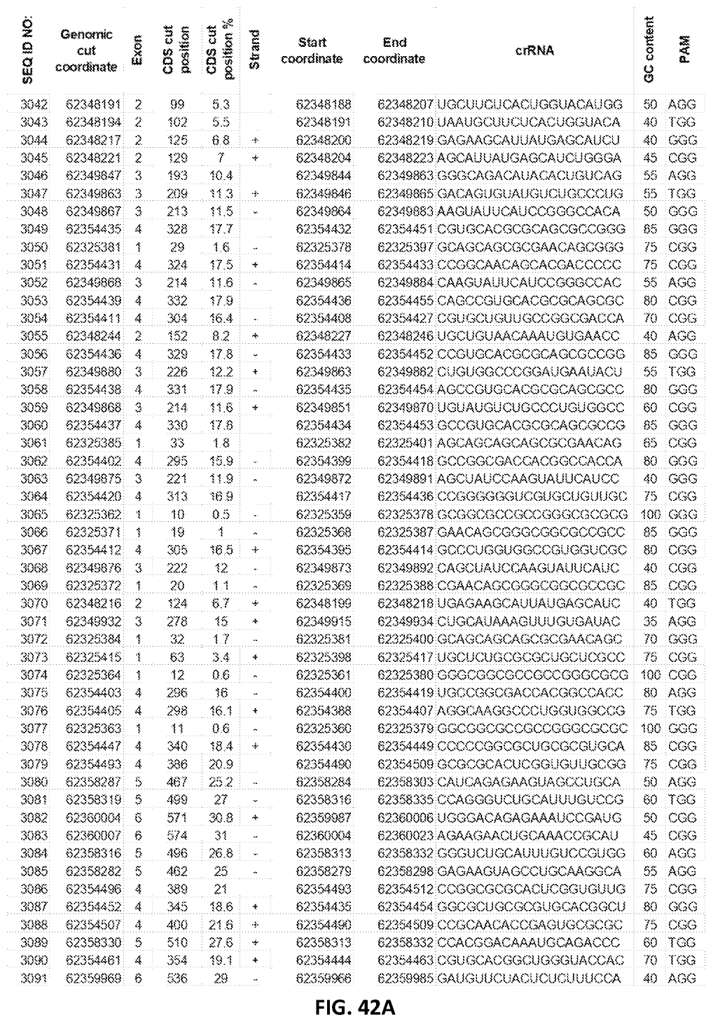

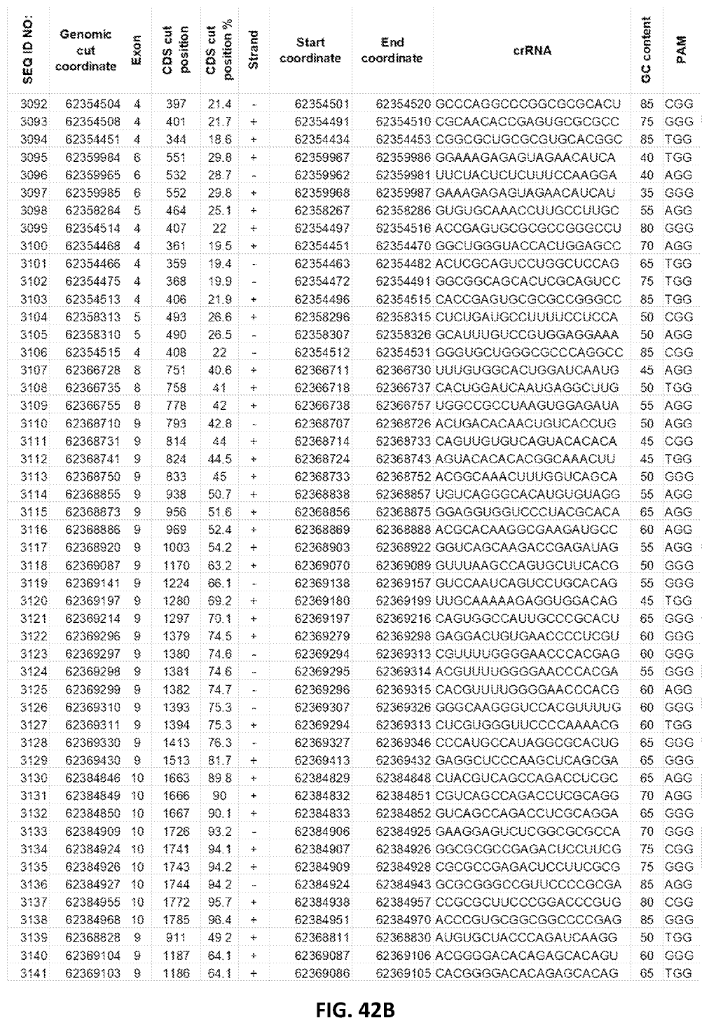

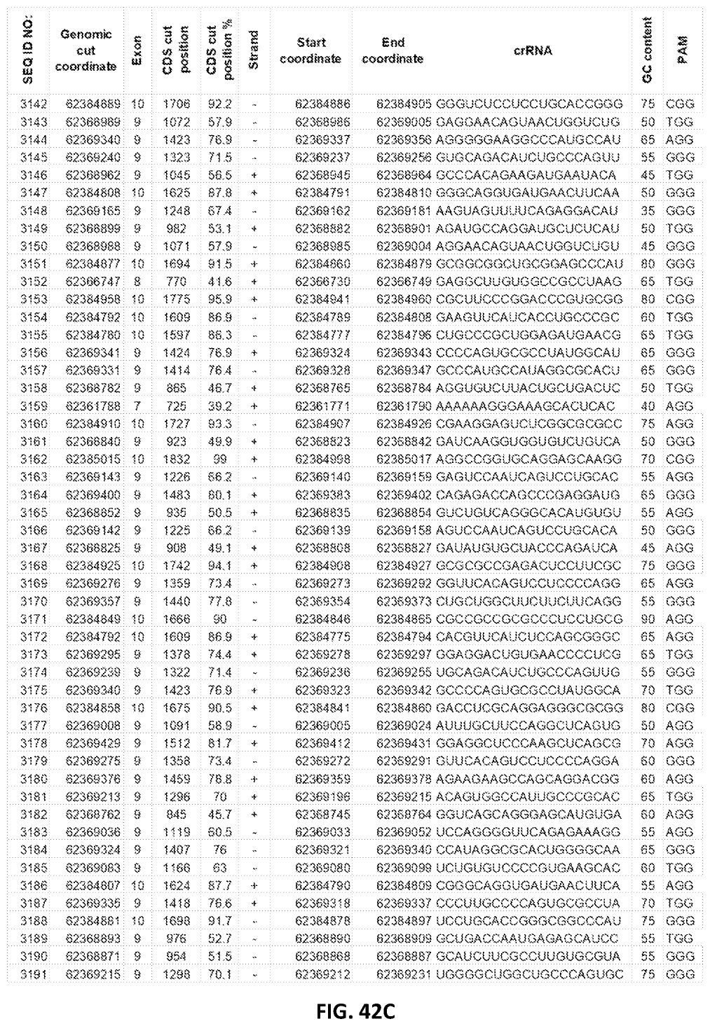

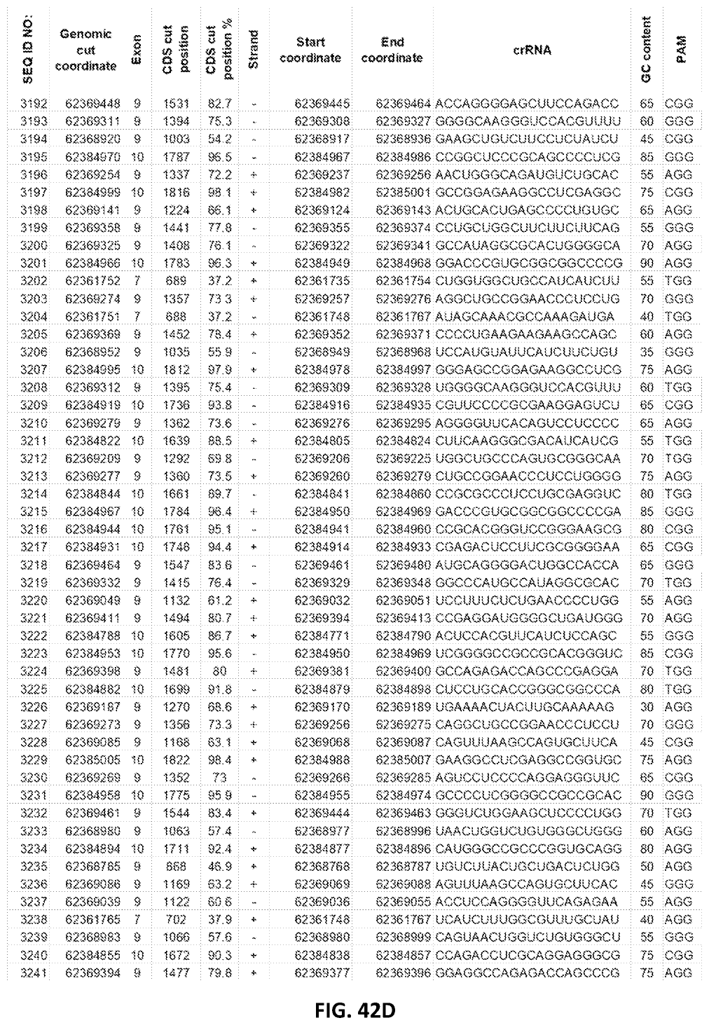

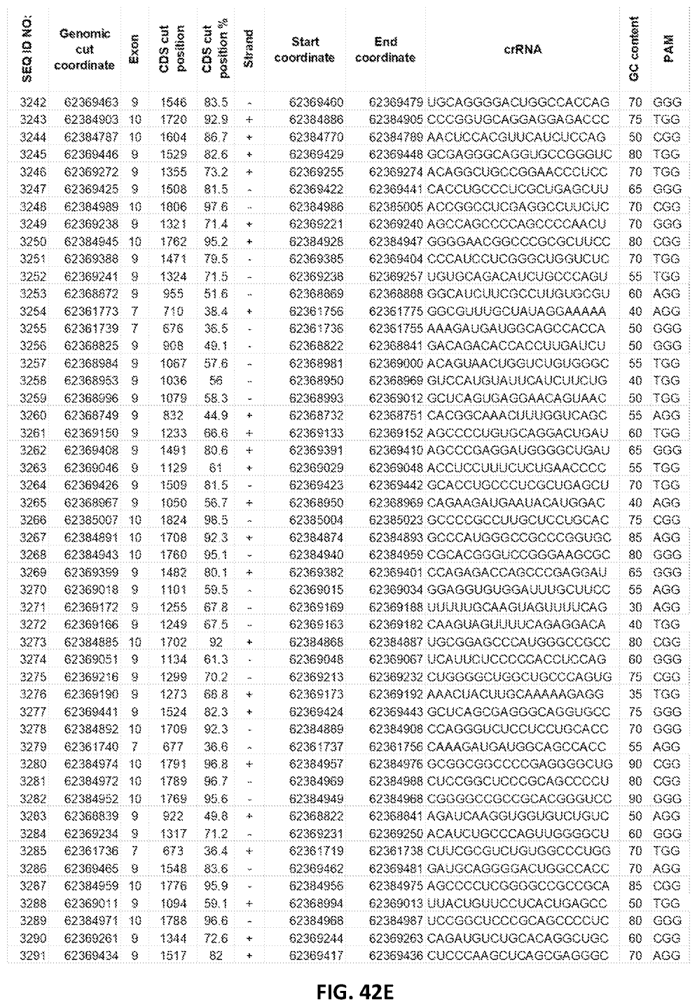

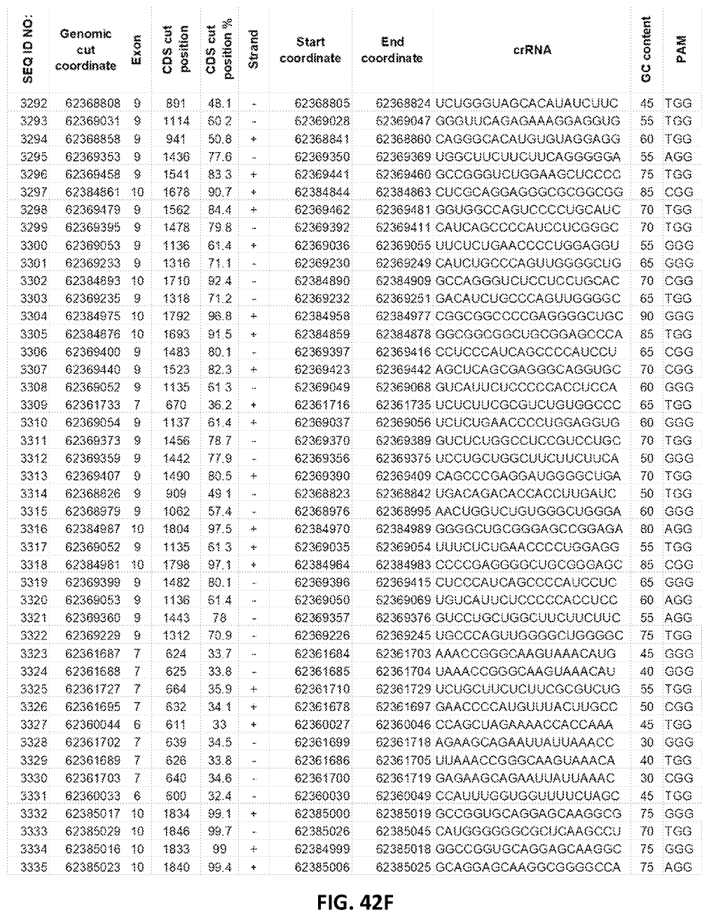

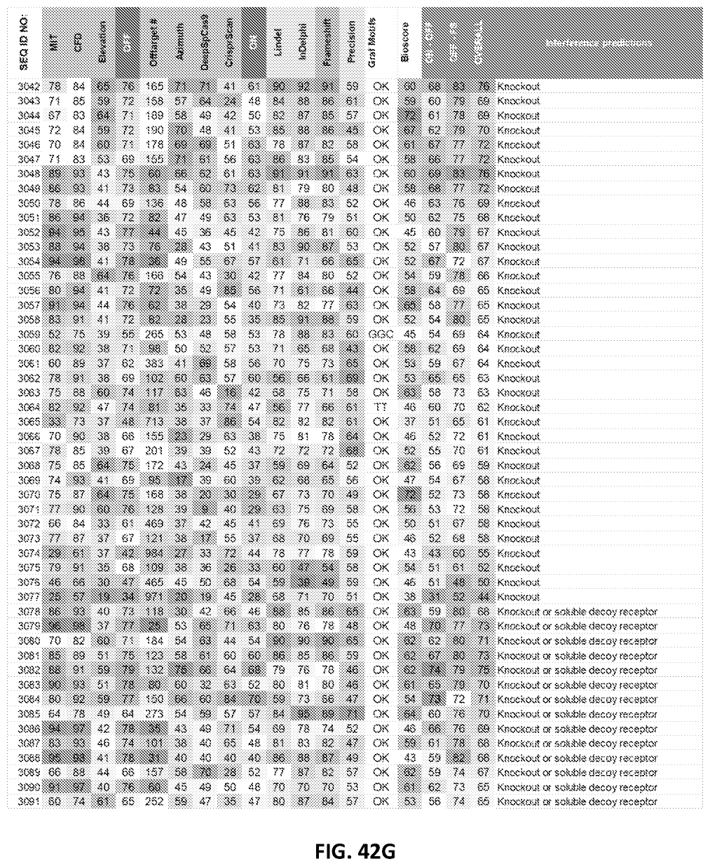

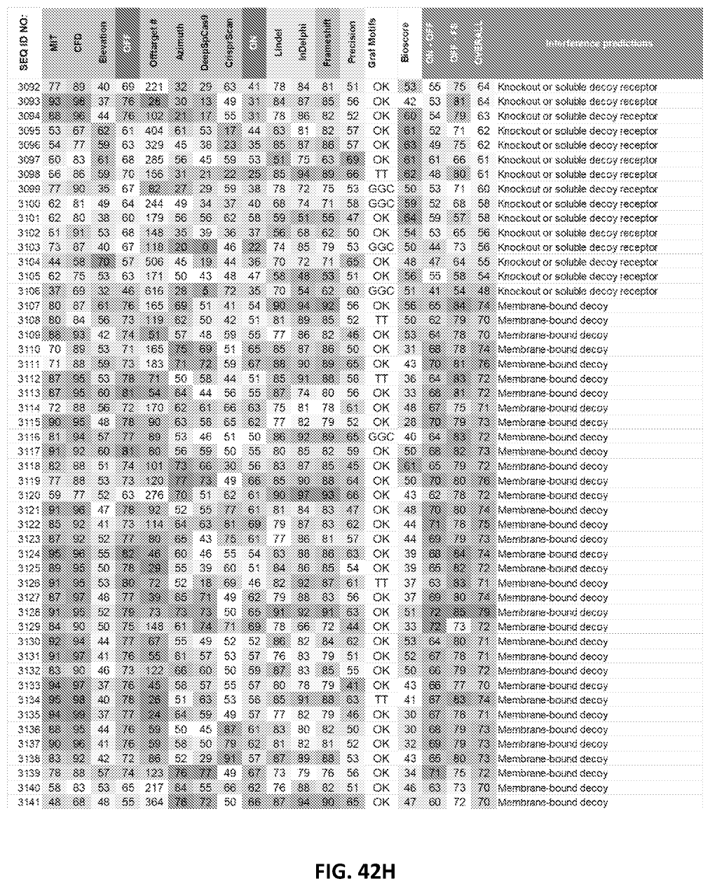

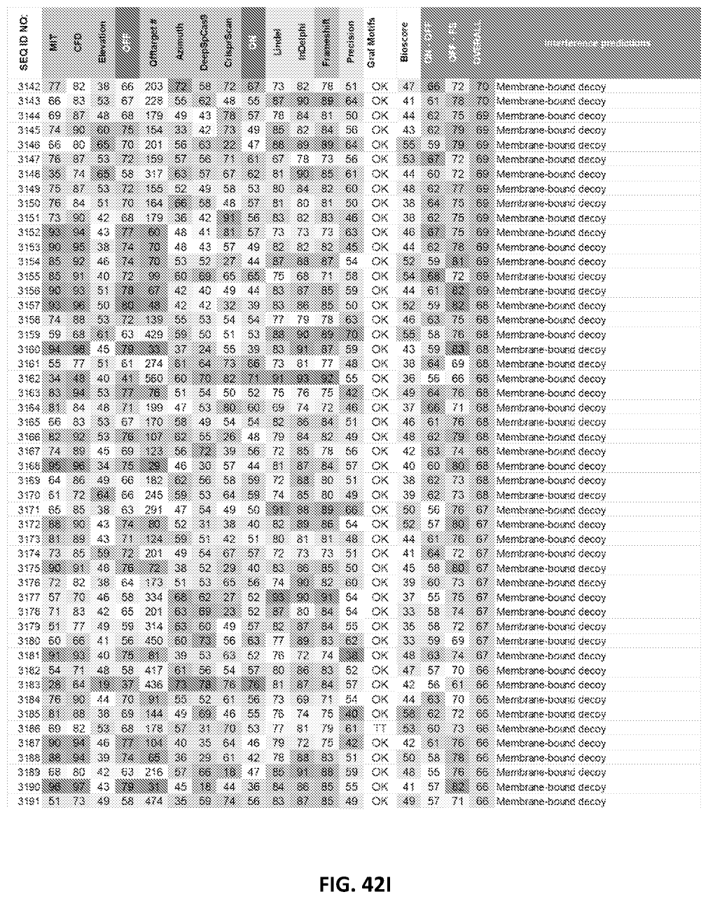

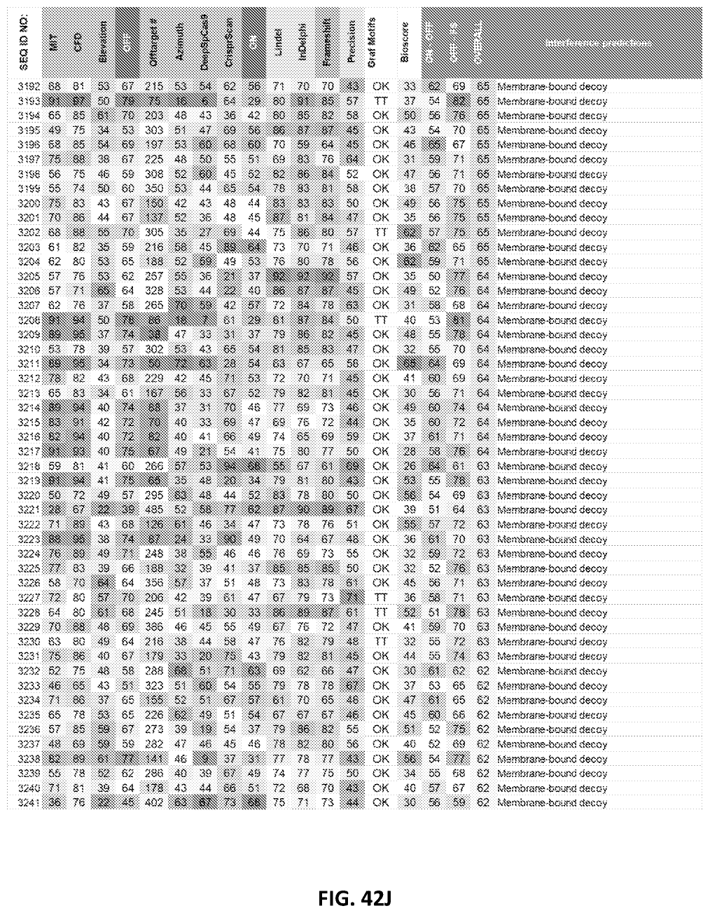

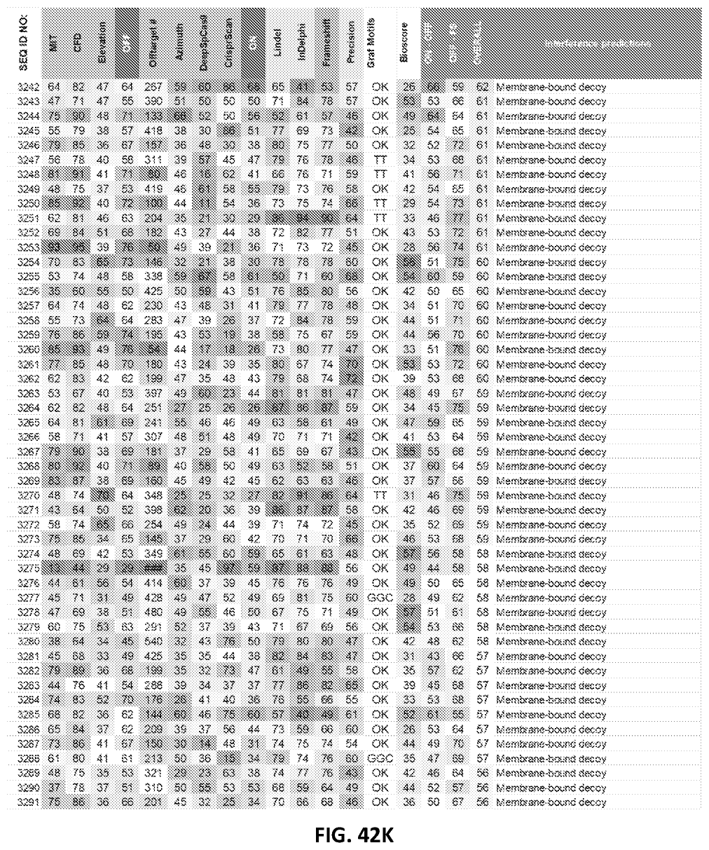

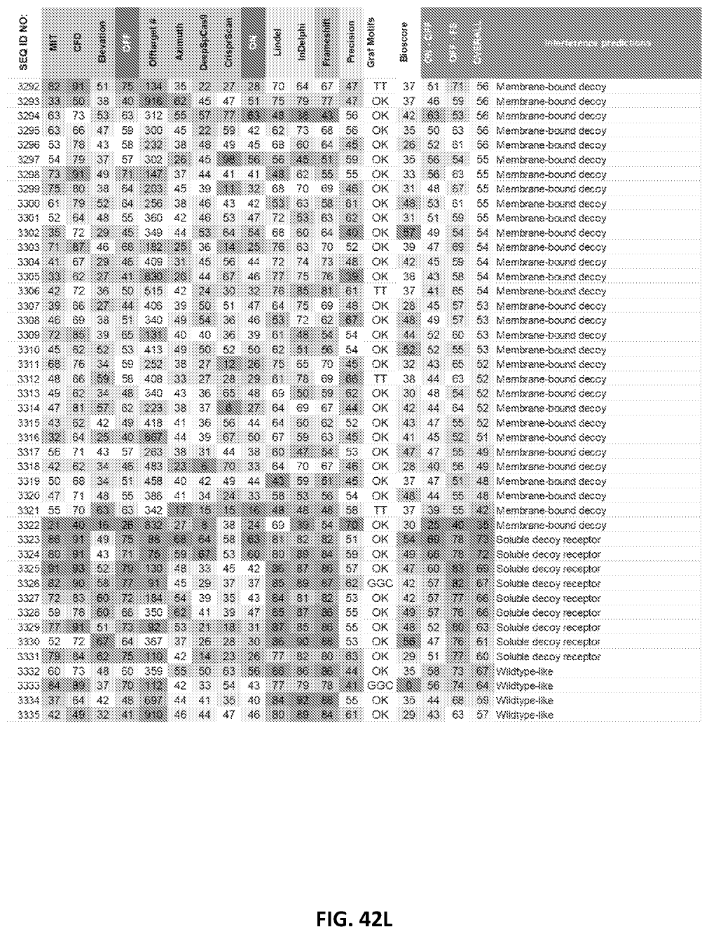

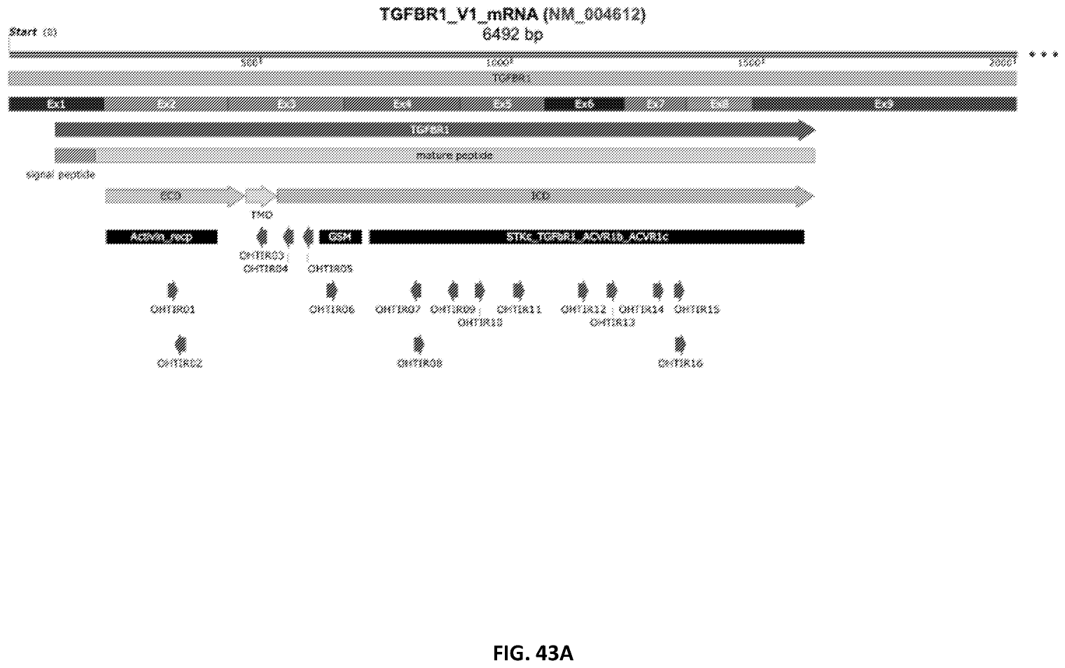

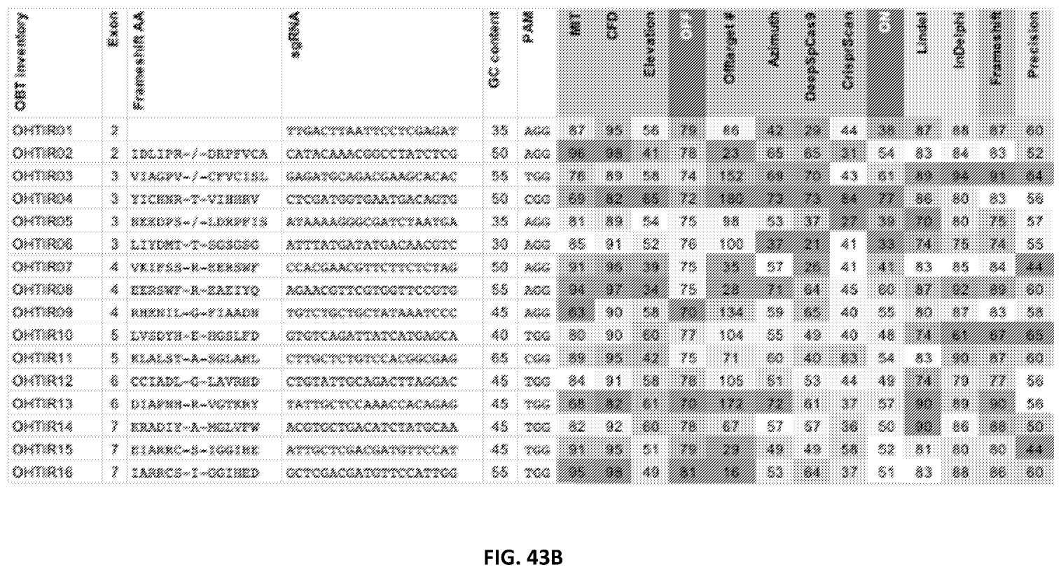

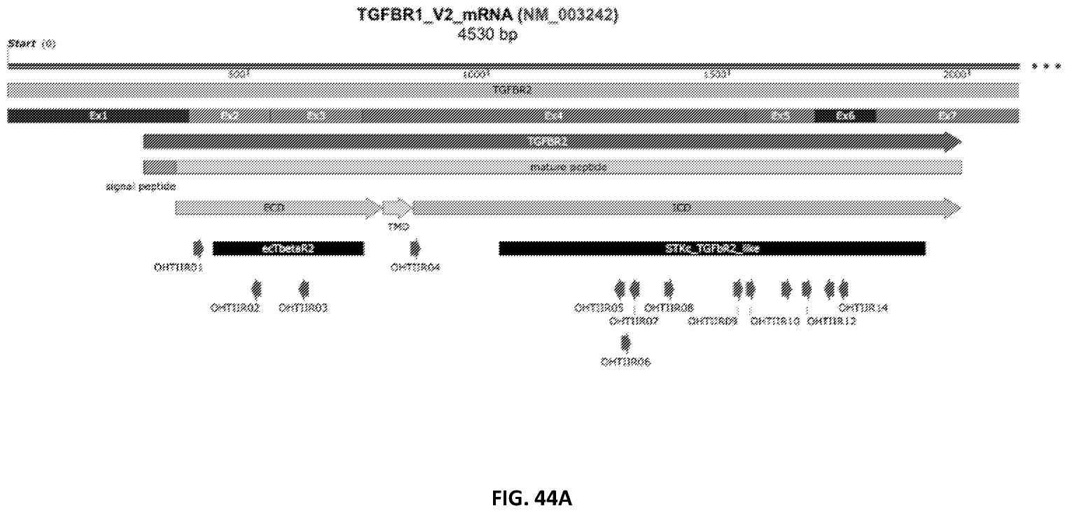

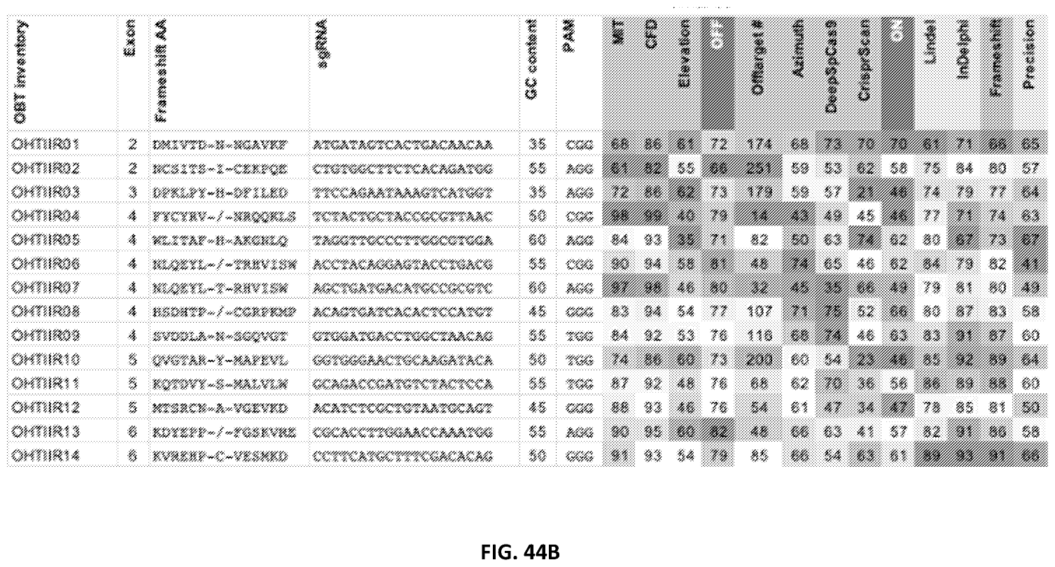

The candidate crRNA sequences were then evaluated for the presence of Graf motifs, TT or GCC present in the 4 PAM proximal bases of the crRNA sequence, as indicated in, e.g., FIGS. 26-42, as either TT or GCC. Graf, R. et al. sgRNA Sequence Motifs Blocking Efficient CRISPR/Cas9-Mediated Gene Editing. Cell Reports, 26 (5), 1098-103 (2019). Graf et al. reported that TT- and GCC-motifs are a hallmark of inefficient sgRNAs. If possible, crRNA with Graf motifs and in particular the GCC motif were avoided. In contrast to the TT motif, the GCC motif remains critical if sgRNAs are synthesized de novo rather than by transcription.

As shown in, e.g., FIGS. 26-42, three consensus scores were then calculated for each crRNA sequence. The “ON-OFF” score represents the mean of the “ON” and “OFF” score. The “OFF-FS” score represents the mean of the “OFF” and “Frameshift” score. The OVERALL score represents the mean of the “ON”, “OFF” and “Frameshift” score. Additionally, for human only, VBC's Bioscore was used to predict whether a possible in-frame mutation could disturb protein function. This is more likely to occur in conserved genomic DNA sequences coding for critical protein domains. Thus, Bioscore is based on protein domain annotations, phylogenetic conservation, amino acid identities and exon size. Michlits, G. et al. Multilayered VBC score predicts sgRNAs that efficiently generate loss-of-function alleles. Nature Methods 17, 708-716 (2020). The final criteria for selecting candidates is mainly based on the OVERALL score (in most cases >70), the relative low counts of potential off-targets (in most cases <200 off-targets), the absence of Graf motifs (if possible) and the genomic cut position within the coding sequence to produce knockouts or truncated proteins with reduced functionality (e.g. decoy receptors),

Finally, the IL1R1 domain in which each crRNA guides editing was determined based on the nucleotide position in the gene. The domain location is used to predict whether the edit will result in a wild-type like protein (edits in the sequence encoding the extreme C-terminus of a receptor that are unlikely to disrupt receptor-mediated signaling), complete knock-out of any functional protein (edits in the sequence encoding the N-terminus and/or essential functional domains), a soluble decoy receptor (edits in the sequence encoding the transmembrane domain of the receptor), or a membrane-bound decoy receptor (edits in the sequence encoding the intracellular signaling domain of the receptor).

Indeed, this in silico analysis established that several candidate sgRNAs were predicted to yield genomic edits that would create a dominant negative (DN) IL1R1, thereby impacting intracellular pro-inflammatory signaling (FIG. 11A). In support of the robustness of these analyses, structural predictions also demonstrated that editing with these particular sgRNAs would yield an expressible form of IL1R1 protein (either soluble or membrane-bound) that cannot induce the ligand's signal (FIG. 11B). Taken together, these predictive studies demonstrated the feasibility of inducing a dominant negative canine IL1 receptor as a unique approach to tamping down inflammation in cells and provided a high-throughput method for validating candidates without the need for time- and resource-intensive brute force methods.

### Example 2: Validation of Dn IL1R1 in Canine Cells

Having designed and analyzed numerous sgRNA candidates that were predicted to edit IL1 receptor to produce a dominant negative reception, the next step was to assess their ability to act in vitro. Of particular interest is whether the preferred (i.e., on-target) edit would be observed in edited cells.

To test this, 80 pmoles of sgRNA candidates were introduced into canine DH82 monocytes via electroporation as part of a ribonucleoprotein (i.e., pre-assembled with 25 pmoles of Cas9 protein at room temperature for at least 5 min). In brief, around 600,000 cells resuspended in 20 μl PKM buffer (2.7 mM KCl, 137 mM NaCl, 10 mM Na2HPO4/1.8 mM NaH2PO4 (pH 7.4) and 50 mM D-Mannitol) were added to the pre-assembled Cas9 ribonucleoprotein complex, transferred into 16-well nucleocuvettes and electroporated with pulse code CM-137 using the 4D nucleofector (Lonza). Electroporated cells were kept in PKM buffer for about 10 min before transferring them into 6-well tissue culture plates with complete culture media. After 2-5 days in culture, cells were dissociated for genomic DNA extraction using the DNeasy Blood & Tissue Kit (Qiagen). sgRNA target regions were amplified by PCR and editing efficacy was then deduced from Sanger sequencing traces using Inference of CRISPR Edits (ICE v1.2). See, Conant, D. et al. (2022). The CRISPR Journal, 5 (1), 123-130.

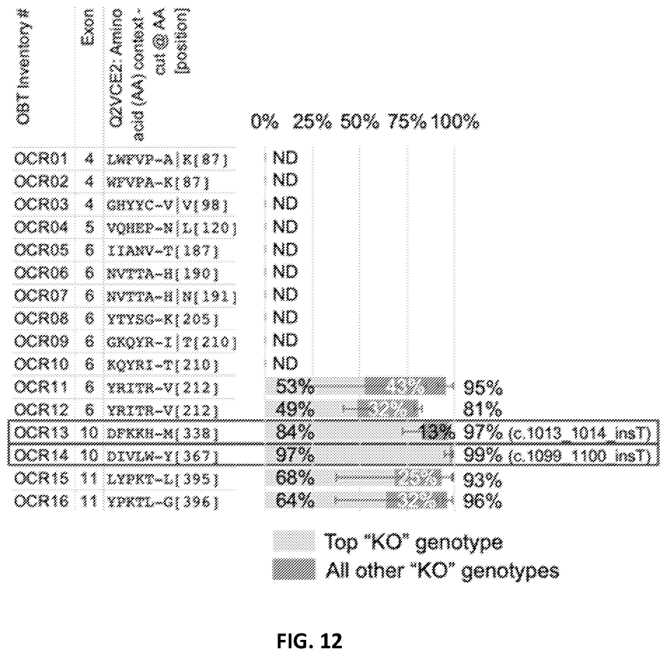

While multiple candidates were indeed able to induce mutation in the IL1R1 gene at high efficiency-including several exhibiting frequencies above 95%-two lead candidates exhibited the ability to consistently induce the same on-target edit in a high percentage of cells (FIG. 12). These results confirmed the robustness of the in silico analysis and suggested that at least OCR13 (SEQ ID NO: 3403) and OCR14 (SEQ ID NO: 3336) are able to produced DN IL1 receptors in canine cells.

### Example 3: Impact of Cas9 Mutants with Enhanced Specificity

With the ultimate aim to eliminate off-target editing without sacrifizing on-target activity, the lead candidate sgRNAs were assembled with different high-fidelity Cas9 variants (i.e. mutated Cas9 nucleases with enhanced specificity; see generally, Slaymaker, I. M. et al (2016). Science, 351 (6268): 84-88; Vakulskas, C. A. et al. (2018). Nature Medicine, 24 (8): 1216-1224) and assessed for frameshift efficacy and precision (i.e. the primacy for single or low number of repair outcomes) in canine DH82 monocytes.

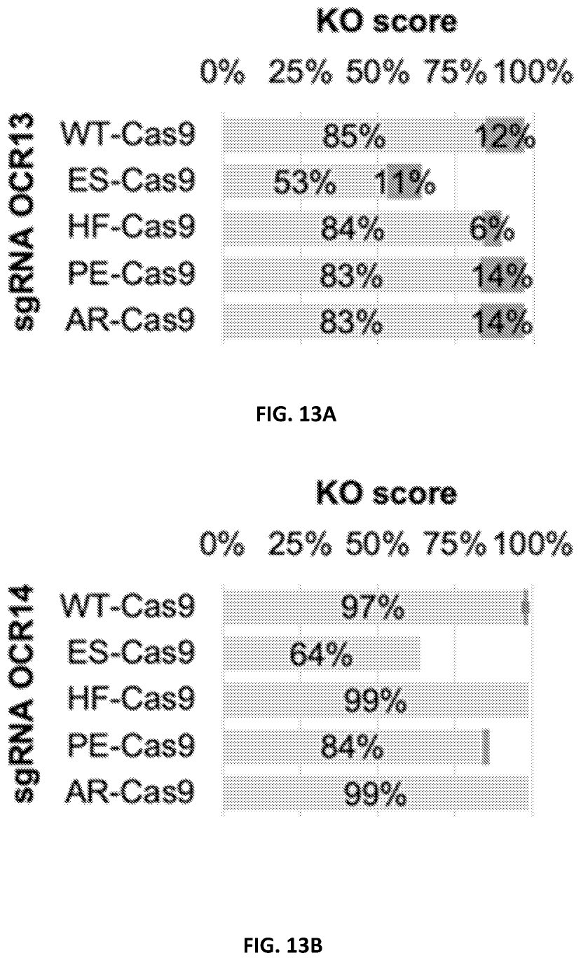

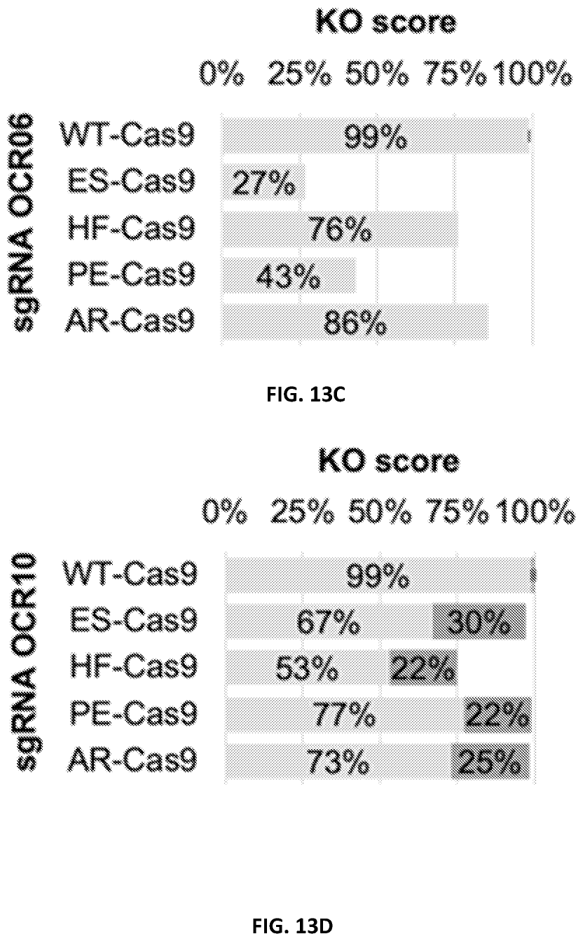

Among the high-fidelity Cas9 nucleases, AR-Cas9 (IDT, Catalogue #1081060; Vakulskas, C. A. et al. (2018). Nature Medicine, 24 (8): 1216-1224) performed best overall with the lead candidate sgRNAs. In comparison to wild-type Cas9 (WT-Cas9), AR-Cas9 reached similarly high frameshift efficacy (>97%) and precision (>83%) when paired with OCR13 (FIG. 13A) and OCR14 (FIG. 13B). The other high-fidelity Cas9 nucleases tested were ES-Cas9 (Growth Factory, eSpCas9 (1.1); Slaymaker, I. M. et al (2016). Science, 351 (6268): 84-88), HF-Cas9 (Invitrogen, Catalogue #A50576) and PE-Cas9 (Merck Life Science, Catalogue #PECAS9-250).

However, AR-Cas9 like all other high-fidelity Cas9 nucleases (ES-Cas9, PE-Cas9 and HF-Cas9) showed decreased editing efficacy with OCR06, as compared to a combination with WT-Cas9 (FIG. 13C). Also, OCR10, which exhibits high precision with WT-Cas9, gives rise to a second frameshift edit at frequency 20-30% with all tested high-fidelity Cas9 nucleases (FIG. 13D). These results, taken as a whole, suggested that pairings between high-fidelity RNA-dependent nuclease and sgRNA would likely need to be done on a case-by-case basis.

### Example 4: Design for IL1RAP/IL1RAP Decoy Proteins

IL1RAP is the co-receptor of all IL1 ligands, including IL33, which activates the IL1RL1/IL1RAP receptor dimer rather than the IL1A/B-specific IL1R1/IL1RAP. Thus, like other IL1R complex proteins, IL1RAP may be another potent mediator of inflammation that can be exploited for therapeutic purposes.

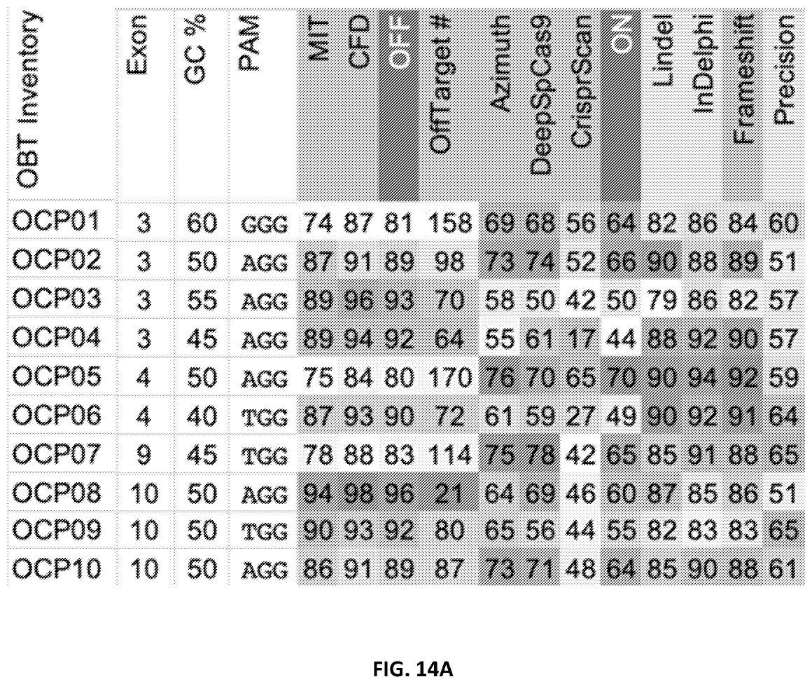

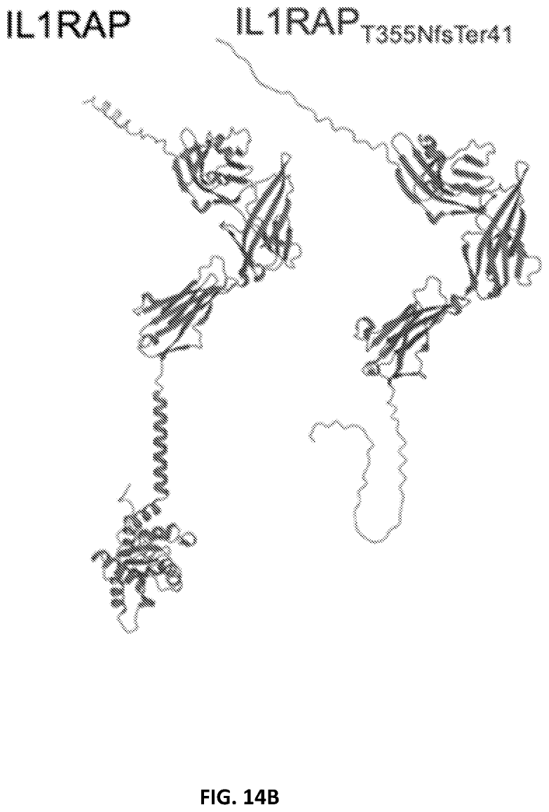

To test this, 10 candidate sgRNAs were designed to target different exons of canine IL1RAP with the objective to either ablate IL1RAP expressing (OCP01 to OCP06) or generate decoy IL1RAP proteins that will not transduce a pro-inflammatory signal (OCP07 to OCP10) (FIG. 14A). For example, OCP07 is predicted to predominantly produce an A duplication in exon 9 of IL1RAP, which shifts the open reading frame at position 355. At the protein level, this causes a Threonine (T) to Asparagine (N) amino acid change and prematurely terminates translation at position 395. Based on AlphaFold2-generated models, this +1 frameshift converts IL1RAP into a soluble IL1RAP decoy (IL1RAP_{T355NfsTer41}) missing the transmembrane and the cytoplasmic TIR domain (FIG. 14B).

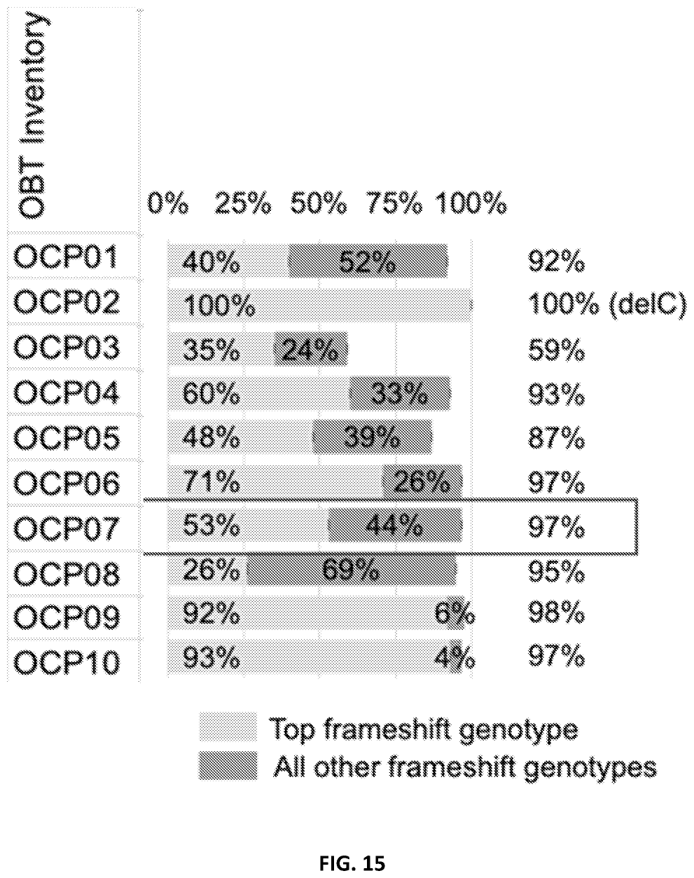

Having done the predictive analysis, the next step was to assess performance in vitro. Candidate sgRNAs were paired with WT-Cas9 and introduced into canine DH82 monocytes via electroporation and editing efficacy and precision assessed by ICE. As predicted with an OVERALL score of 82, OCP02 performed best among the knockout candidates with virtually 100% KO rate due to a single C deletion introducing a premature stop codon at position 32 (M32*) (FIG. 15). All decoy-producing sgRNA candidates exhibited strong editing efficacy (greater than 95%). OCP09 and OCP10 demonstrated the strongest editing primacy with the top edit reaching 89% and 95%, respectively.

### Example 5: Editing of IL1RAP/IL1RAP in Various Canine Cell Types with High Fidelity Nucleases

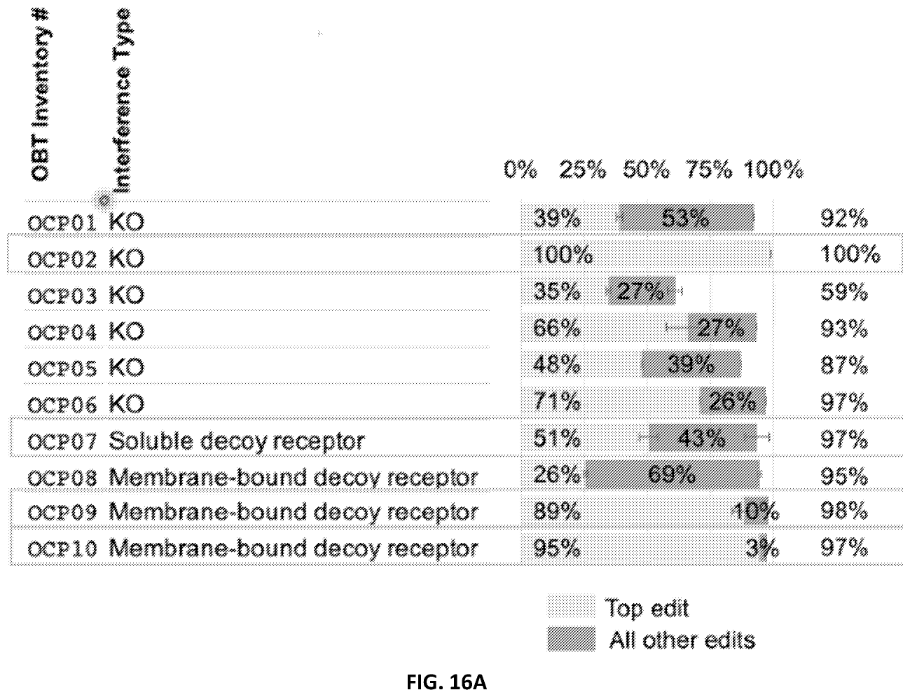

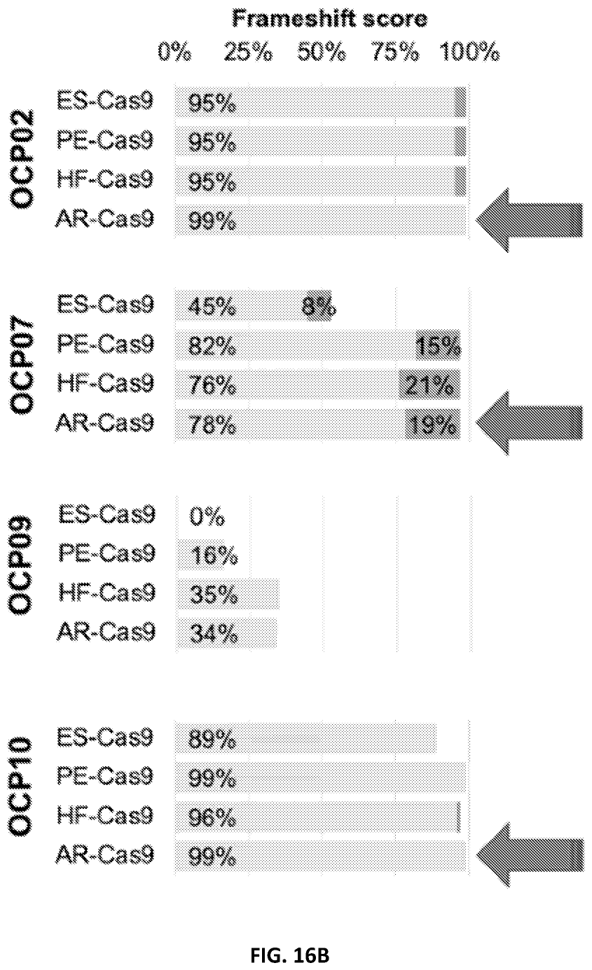

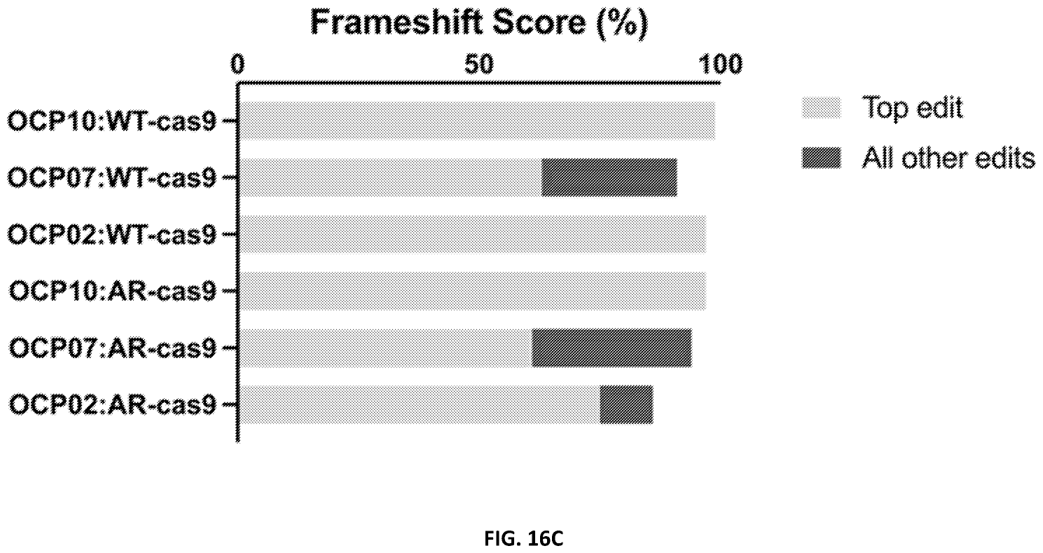

Having designed and validated numerous guides targeting IL1RAP, the next step was to validate them with high-fidelity Cas9 nuclease to mitigate potential off-target effects during the editing process. To do this, one or two sgRNAs with the strongest frameshift scores (FIG. 16A) for each type of interference (e.g., knockout or decoy receptor) were selected and paired with different high-fidelity Cas9 nucleases to see which, if any, would show similar on-target activity to WT-Cas9, while potentially reducing undesirable off-target effects.

Indeed, for each selected sgRNA, the editing efficacy in canine DH82 monocytes remained strongly dependent upon which high-fidelity Cas9 nuclease was used (FIG. 16B). Notably, AR-Cas9 most consistently showed high frequency of the desired edit in canine IL1RAP. This was particularly true in the case of OCP02, OCP07, and OCP10. However, this was effect was not universal, as the frameshift score for OCP09 was decreased when paired with AR-Cas9. OCP02, OCP07 and OCP10 also performed well in a head-to-head comparison of WT-Cas9 and AR-Cas9 in canine synovial fibroblasts (FIG. 16C). As such, high-fidelity RNA-dependent nucleases appeared to generally impact editing efficiency, though the magnitude of such effects will likely vary depending upon the nuclease used and to a lesser extent the cell type to be edited.

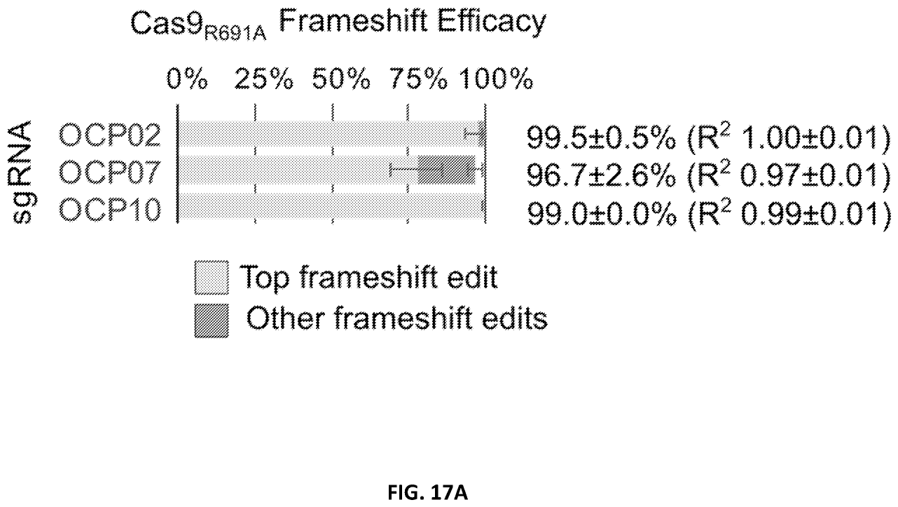

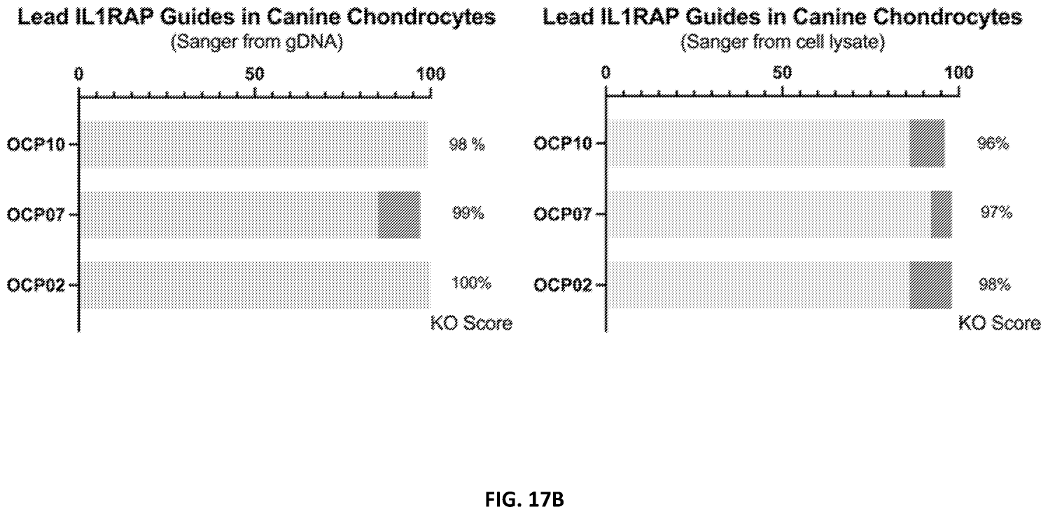

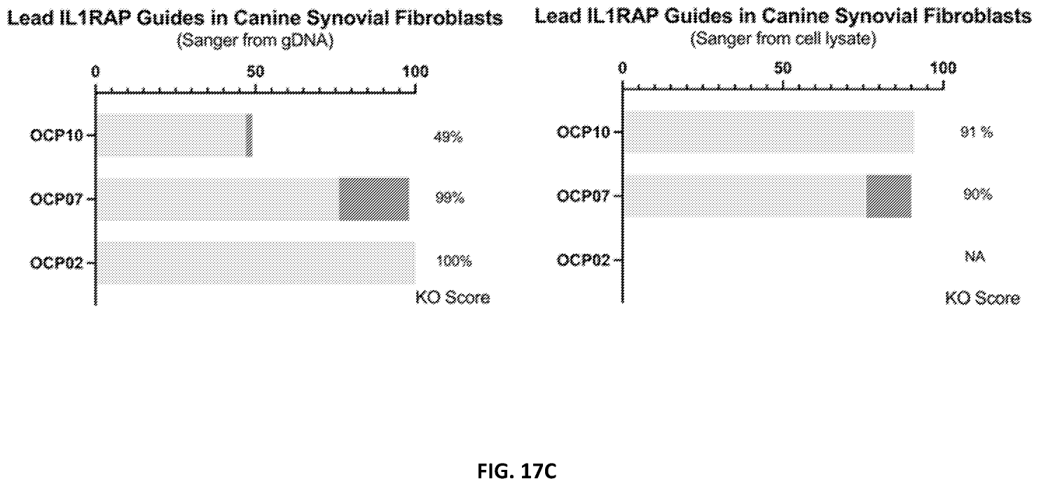

Having observed strong on-target activity when pairing sgRNA with high-fidelity Cas9 nucleases, the next step involved expansion of the assay to include numerous relevant canine cell types, such as DH82 monocytes, chondrocytes, and synovial fibroblasts. Specifically, the same select sgRNAs were used with AR-Cas9 in DH82 monocytes, where a strong preference for each desired edit was again observed (FIG. 17A). Similar editing efficacies were also observed in chondrocytes (FIG. 17B) and synovial fibroblasts (FIG. 17C). This result demonstrated that consistently high editing efficacies and precision for a given sgRNA and high-fidelity RNA-dependent nuclease pairing was achieved across various cell types.

### Example 6: Editing of IL1R1 Renders Canine Synoviocytes Unresponsive to IL1B Treatment

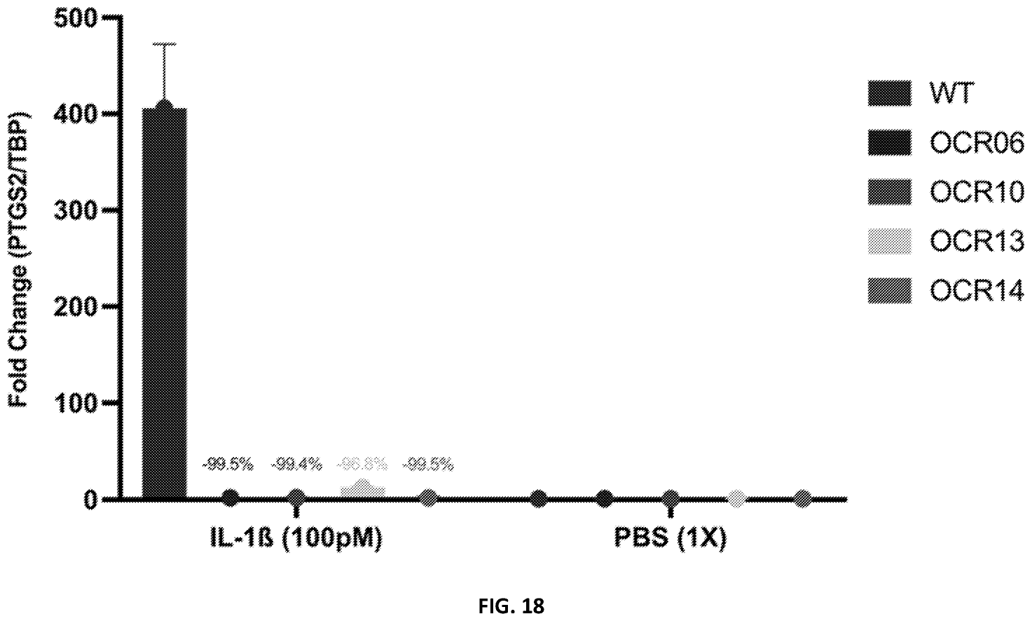

Having designed and validated numerous guides targeting IL1R1, the next step involved functional testing of edited cells. As part of this testing, around 300,000 edited canine synoviocytes were treated with 100 μM human recombinant IL1β for 4 hours. Reverse transcription followed by quantitative PCR (RT-qPCR) on extracted total RNA was carried out to verify whether human recombinant IL1β can stimulate the transcription of the inflammatory marker prostaglandin-endoperoxide synthase 2 (PTGS2/COX-2). No observable transcriptional upregulation of PTGS2/COX-2 was detected, likely because canine IL1R1 does not have a high enough affinity for human IL1β at 100 μM. Indeed, upon repeating of the protocol with recombinant canine IL1B (R&D Systems, 3747-CL), robust upregulation was observed (FIG. 18). However, edited canine cells exhibited no such upregulation, suggesting that IL1β signaling had been disrupted. Both types of IL1R1 interference, knockout (OCR06/OCR10) or decoy receptor conversion (OCR13/OCR14), repressed PTGS2 transcription by 97% or more, which was line with the observed genetic frameshift score.

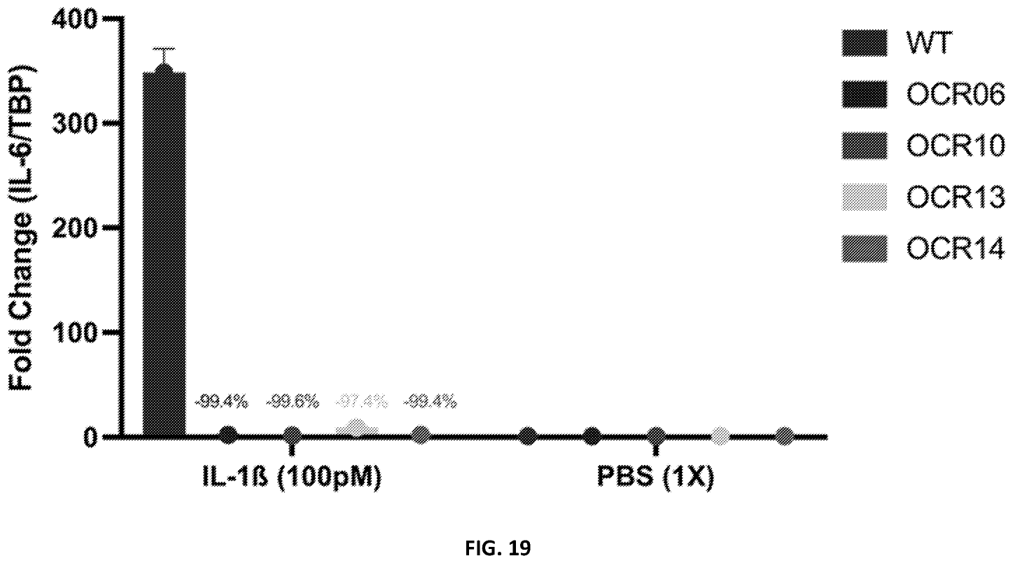

Because of the well-established relationship between IL1 receptor activation and additional pro-inflammatory transcriptional responses, such as the upregulation of IL6 (see, Attur, M. G. et al. (1998). Proceedings of the Association of American Physicians, 110 (1): 65-72), the same assay was used to determine the effect of IL1R1 editing on this pro-inflammatory response following IL1ß treatment. Similar to PTGS2/COX-2, unedited cells exhibited robust upregulation of IL6 after treatment, while no edited cells exhibited this response (FIG. 19). Taken together, these results showed that edited cells had lost the ability to respond to IL1β treatment.

### Example 7: Editing of IL1RAP/IL1RAP Reduces Cox-2 Upregulation in Canine Monocytes Following Exposure to Multiple Inflammatory Triggers

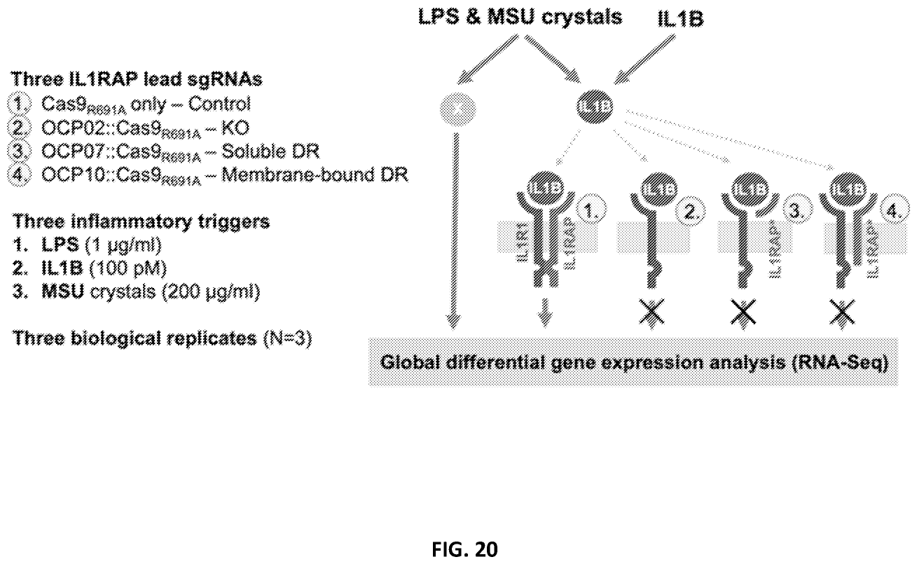

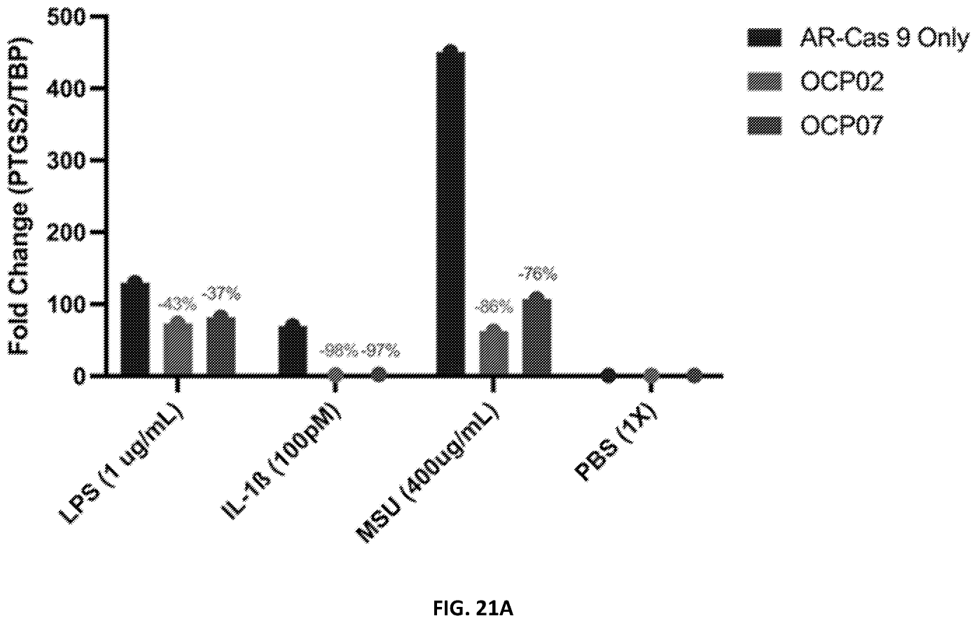

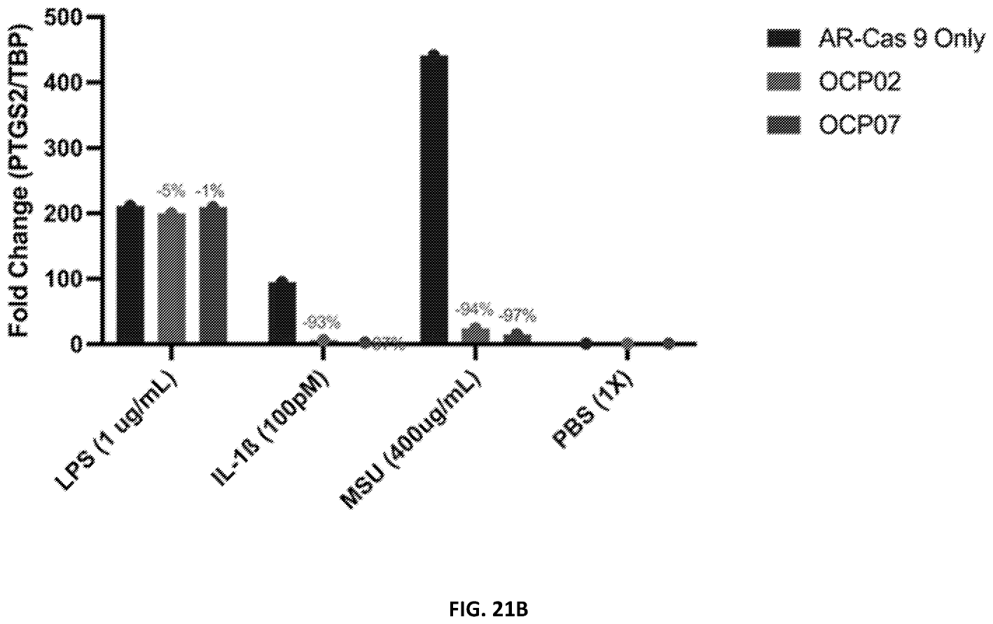

To understand the potential nexus between a dysfunctional IL1RAP and reductions in pro-inflammatory signaling, the transcriptional response to various inflammatory triggers, such as bacterial lipopolysaccharides (LPS), monosodium urate (MSU) crystals and IL1B, were investigated in canine control and IL1RAP-edited DH82 monocytes (biological triplicates, N=3) using bulk RNA-Seq and differential expression analysis DESeq2 (FIG. 20). All edited cell lines were genotyped, and all CRISPR-induced frameshift scores perfectly aligned with previous efficacy readings. Cells were exposed to saline solution PBS (negative control), 1 μg/ml LPS, 100 μM IL1ß or 400 μg/ml MSU crystals, and total RNA was extracted after 4 and 24 hours. IL1RAP genotypes and inflammatory induction of PTGS2 were verified in control and edited cells (FIG. 21).

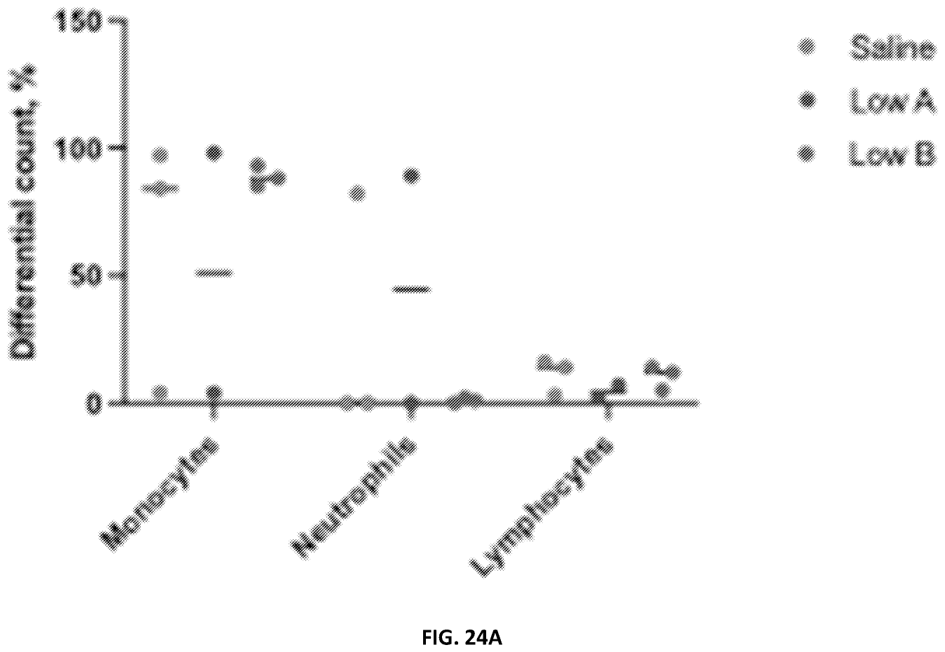

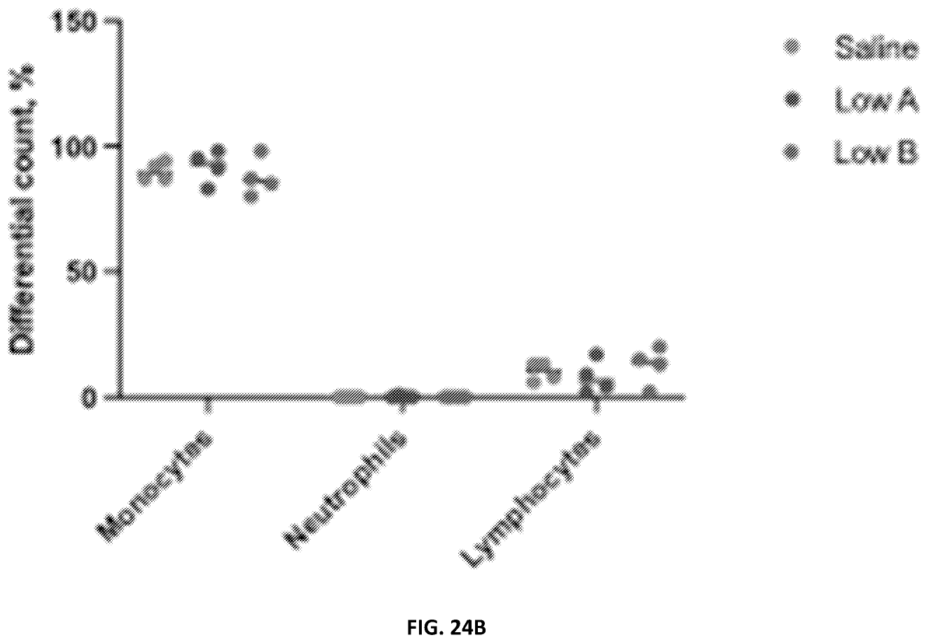

Prior to performing RNA-Seq, the transcriptional effect of inflammatory triggers was validated by RT-qPCR. Stimulation with LPS resulted in strong PTGS2 upregulation at both 4 hours (FIG. 21A) and 24 hours (FIG. 21B). The edited canine cells exhibited similar levels of upregulation, particularly at 24 hours, suggesting that the loss of IL1RAP did not impact LPS-mediated inflammatory signaling, which canonically occurs through engagement of Toll-like receptors, such as TLR2 (see generally, Erridge, C. (2010). Journal of Leukocyte Biology, 87 (6): 989-999.). By contrast, while treatment with either IL1β or MSU also stimulated upregulation in control cells, the edited cells demonstrated abrogation at 4 h and, in particular, at 24 h with PTGS2 induction being repressed by 93% or more. Under control conditions (i.e., PBS treatment) no induction was observed at either time point, and, as observed for IL1R1, similar levels of abrogation of inflammatory signaling were observed whether the cells were edited to generate a knockout (OCP02) or a decoy receptor (OCP07).

### Example 8: Editing of IL1RAP/IL1RAP Reduces Responsiveness of Various Canine Cell Types to IL1B Treatment

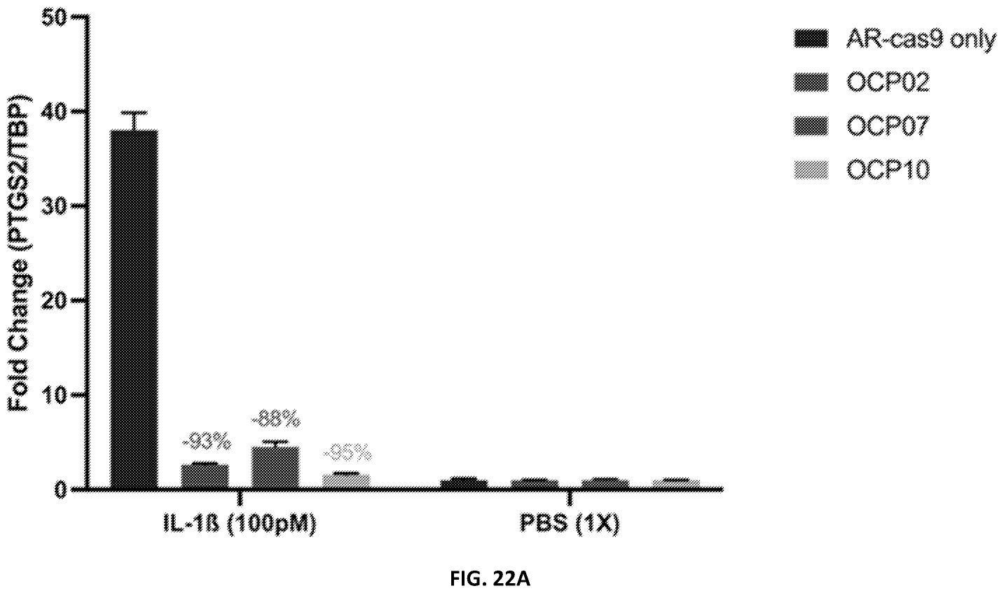

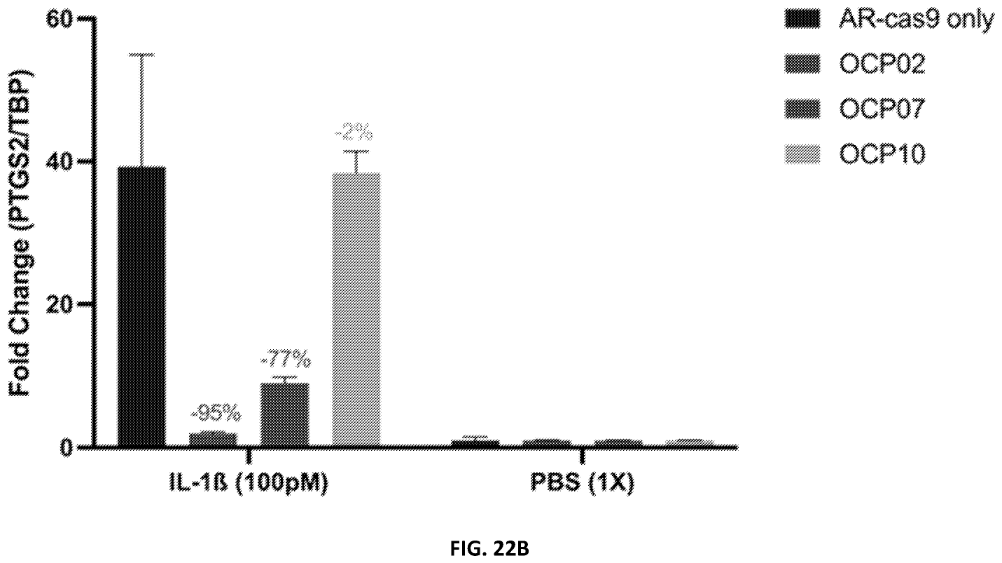

Having designed and validated numerous sgRNAs targeting IL1RAP and having observed reductions in pro-inflammatory signaling at the transcriptional level in canine monocytes, the functional assays continued, this time in edited canine chondrocyte and synoviocytes. As part of this testing, around 300,000 edited canine synoviocytes were treated with 100 μM recombinant canine IL1β for 4 hours. Reverse transcription followed by quantitative PCR (RT-qPCR) on extracted total RNA was carried out to assess the transcriptional impacts of IL1B. Indeed, robust upregulation of PTGS2 was observed in control chondrocytes (FIG. 22A). However, edited canine chondrocytes exhibited no such upregulation, suggesting that IL1β signaling had been disrupted by the interference of IL1RAP, whether the cells were edited to generate a knockout or a decoy receptor. Similarly, control synoviocytes responded to treatment (FIG. 22B). However, the edited canine synoviocytes exhibited differential upregulation, with strong abrogation of IL1β signaling observed when the cells were edited to generate a knockout or a soluble decoy receptor (OCP07). Interestingly, generation of a membrane-bound decoy (OCP10) IL1RAP had little impact on this transcriptional upregulation in synoviocytes. This was likely due to inefficient IL1RAP editing in canine synoviocytes (see FIG. 17C). Repeat experiments are currently underway. These results demonstrated that editing of IL1RAP in various cell types can impact function in response to IL1β exposure.

### Example 9: LNP-Based Delivery of mRNA in Various Canine Cell Types

To this point, nearly all validation experiments had been conducted via electroporation of cell cultures to introduce RNA-dependent nucleases and sgRNAs.

However, to assess the suitability of using lipid nanoparticle (LNP) to deliver mRNA to mammalian cells, LNPs from PNI containing Trilink Firefly Luciferase mRNA were exposed to canine synovial fibroblasts for 8 hours and canine chondrocytes for 24 hours at increasing concentrations (based on a stock 0.1 mg/mL solution). At each time point, a luciferase and Bradford assay were conducted to detect and normalize cellular luciferase activity.

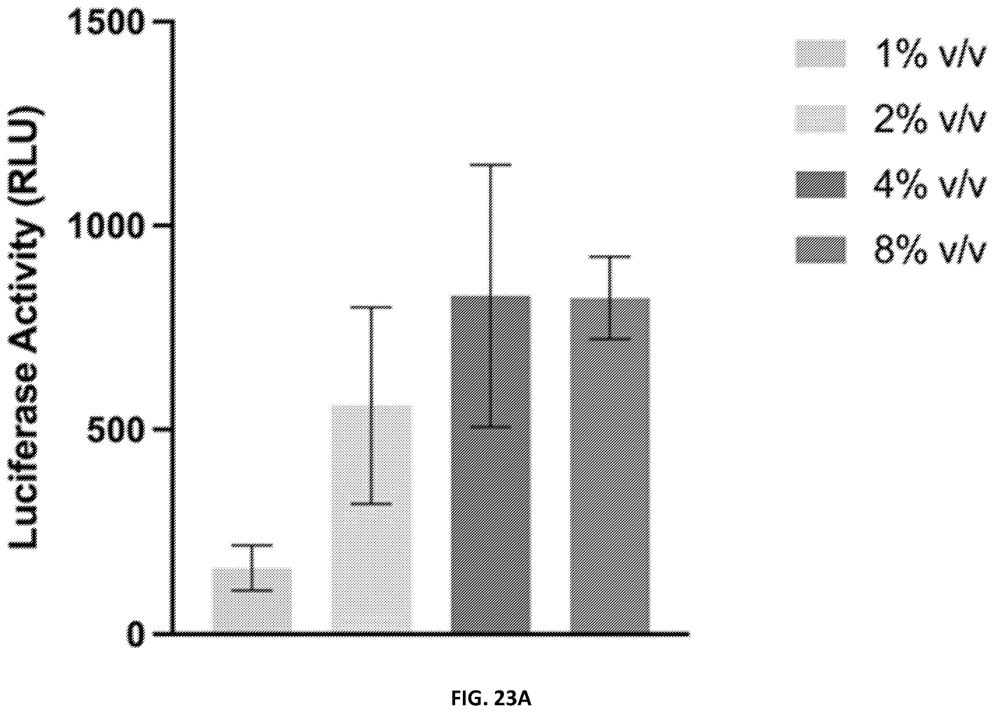

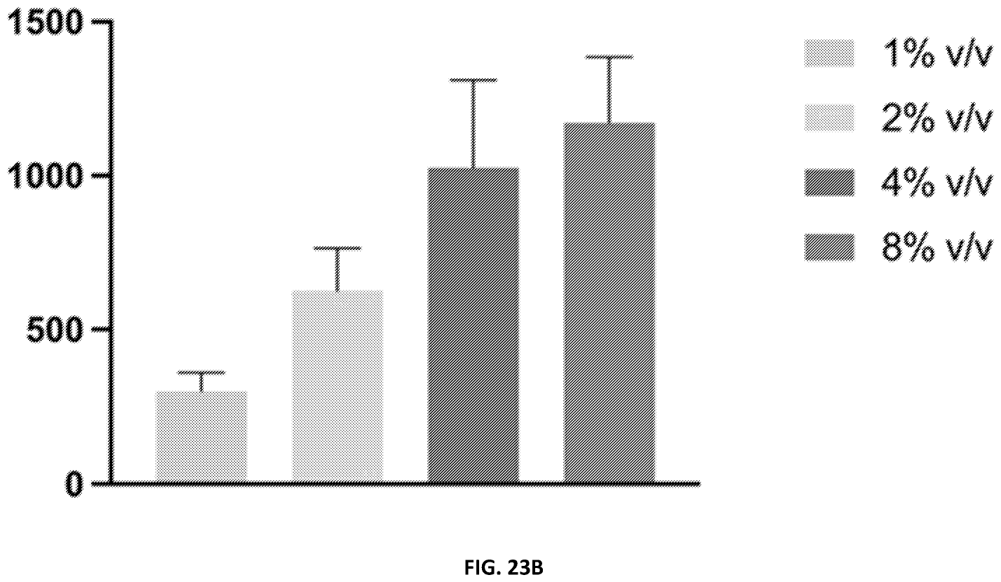

For synovial fibroblasts, increasing luciferase activity was observed with an increase in LNP concentrations from 1% (v/v) to 4% (v/v); however, no statistically significant difference was observed between 4% (v/v) and 8% (v/v) (FIG. 23A). For chondrocytes, similar dose dependency was observed (FIG. 23B). These results showed that the LNP formulation is competent in delivering its cargo in the form of mRNA into therapeutically relevant cells.

Further tests were also conducted to show the safety of these LNP formulations in an organism, particularly in dogs. Low doses (0.025 mg/ml) of LNP compositions (or saline control) were injected into healthy joints of dogs after performing baseline cytology studies on synovial fluid to determine the baseline counts of monocytes, neutrophils, and lymphocytes (FIG. 24A). The same cytology tests were undertaken again four weeks after injection and showed no appreciable differences from the baseline results in the canine synovial fluid (FIG. 24B). Further, no changes in relative counts for any one cell type were observed post injection. Together, these results demonstrated that LNPs can be used to introduce mRNA into multiple canine cell types, while canine injection studies showed that use of these LNPs do not stimulate in vivo inflammatory responses in mammals.

### Example 10: Differential Transcriptional Responses in IL1RAP/IL1RAP-Edited Canine Monocytes are Attributable to the Interference of the Gene

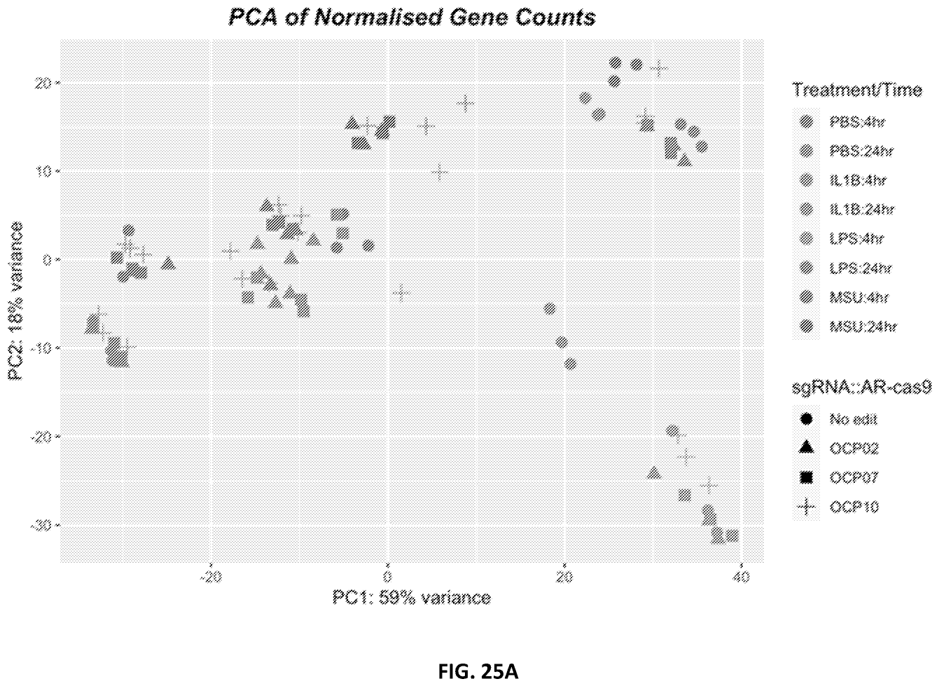

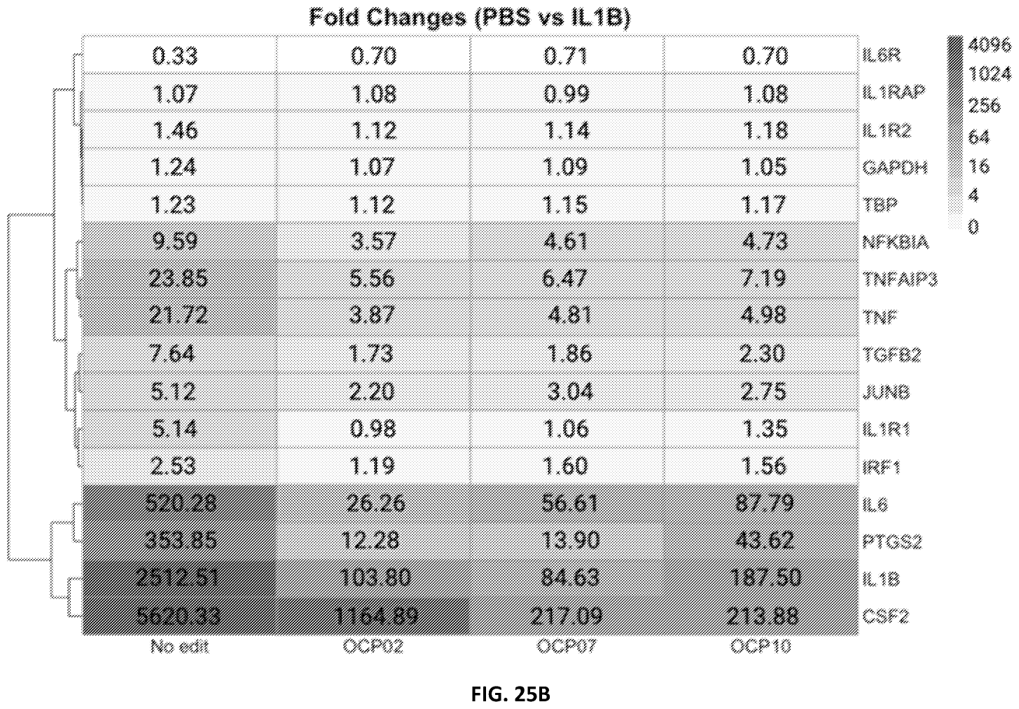

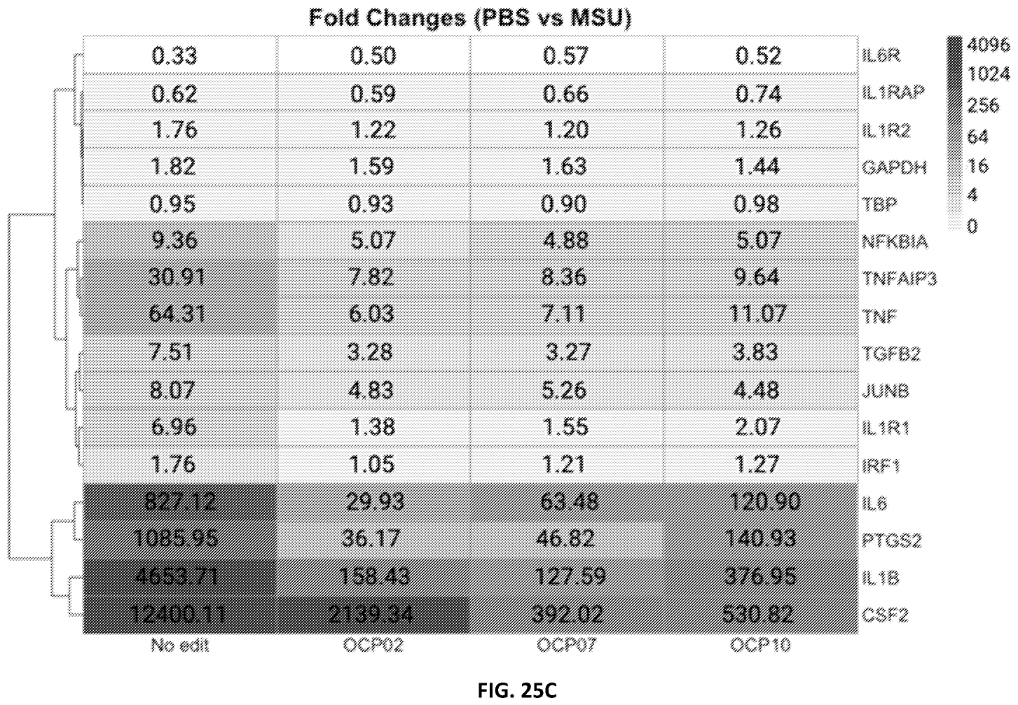

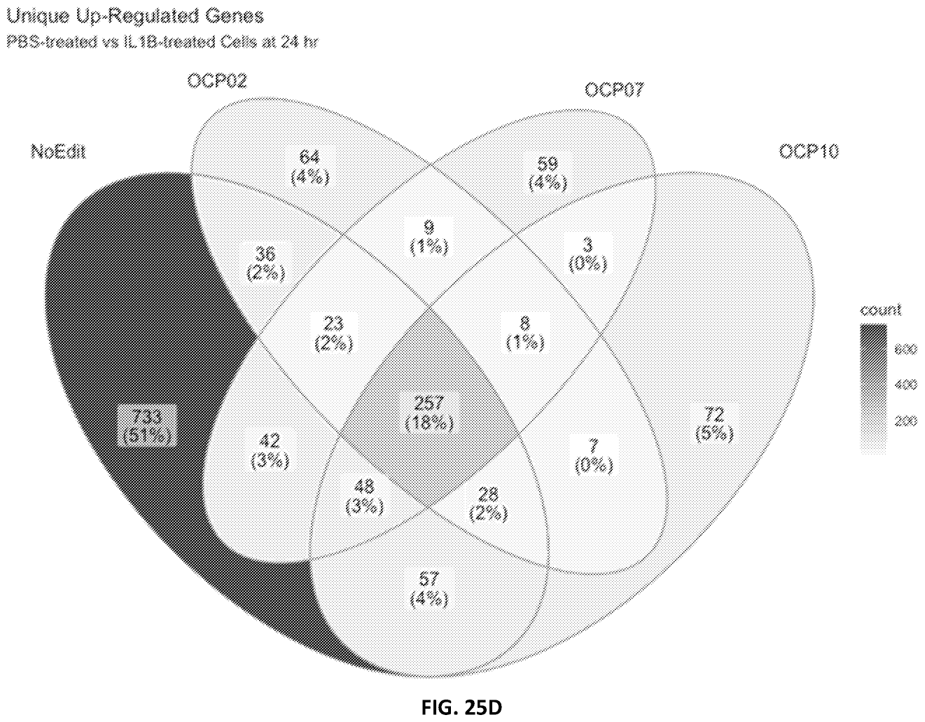

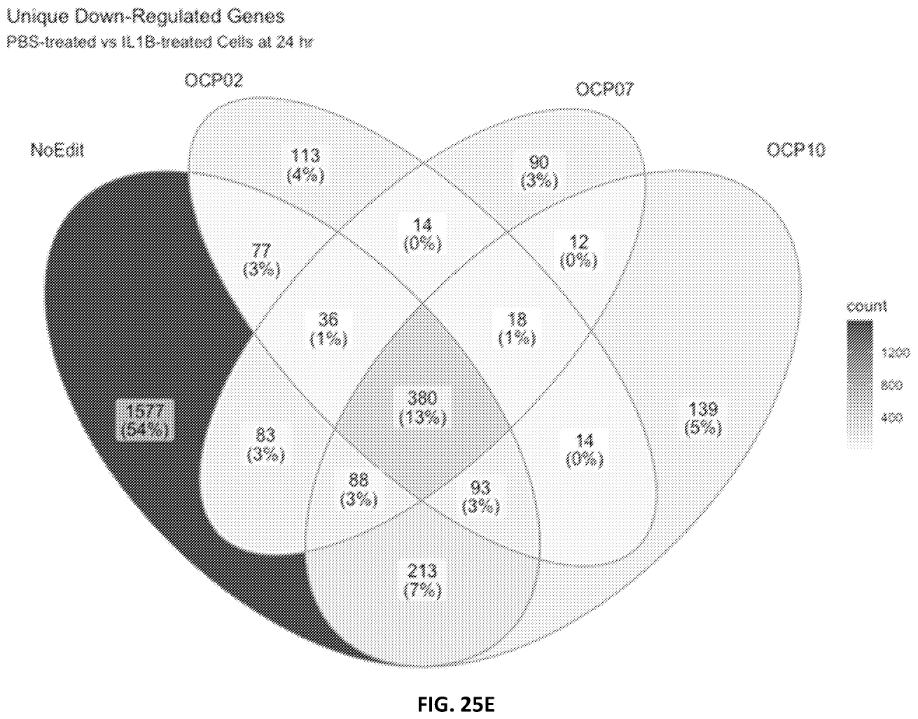

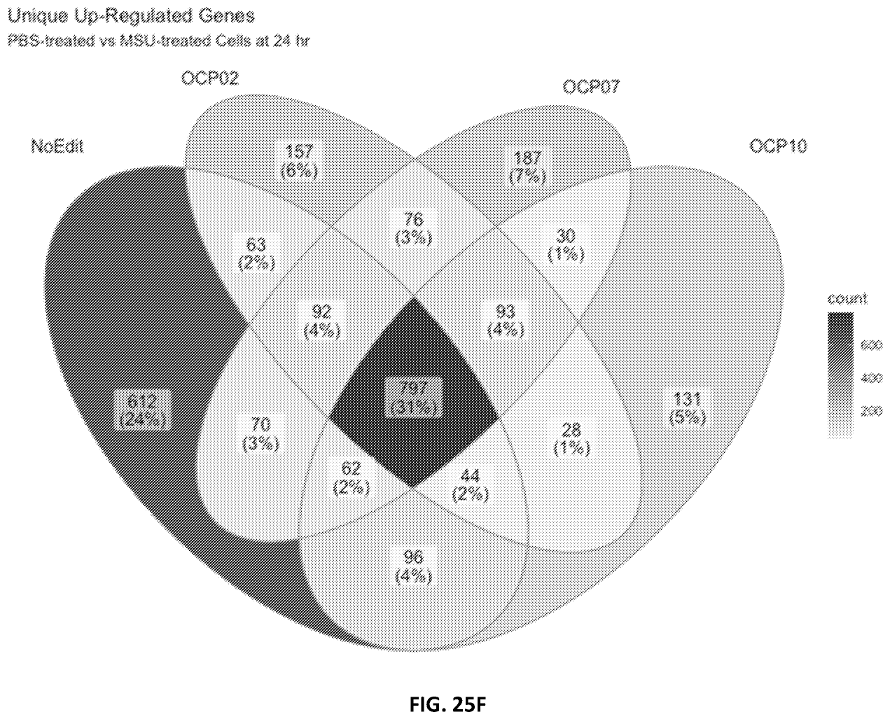

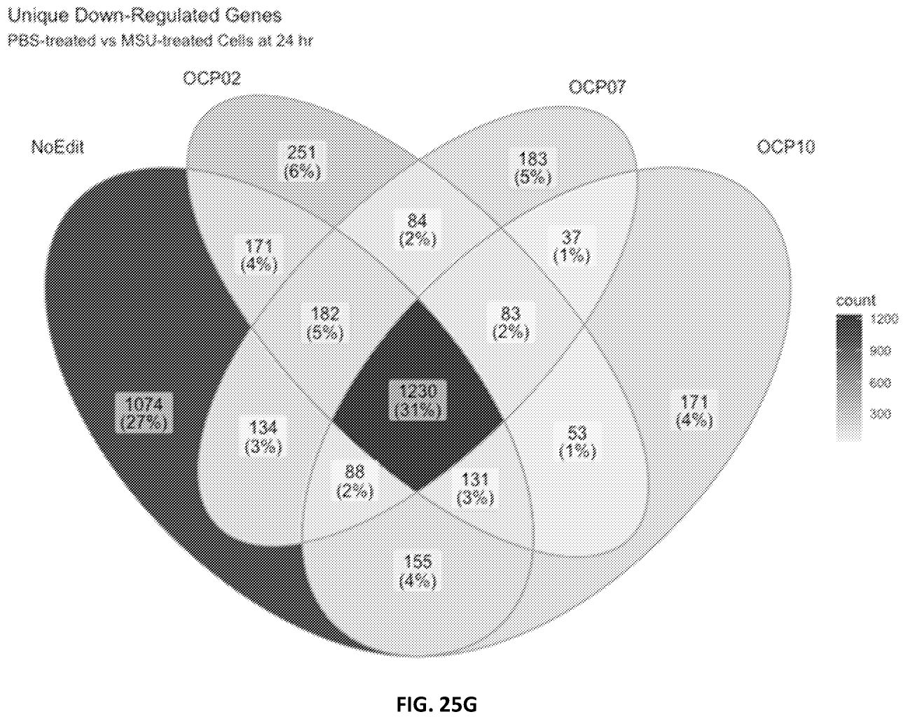

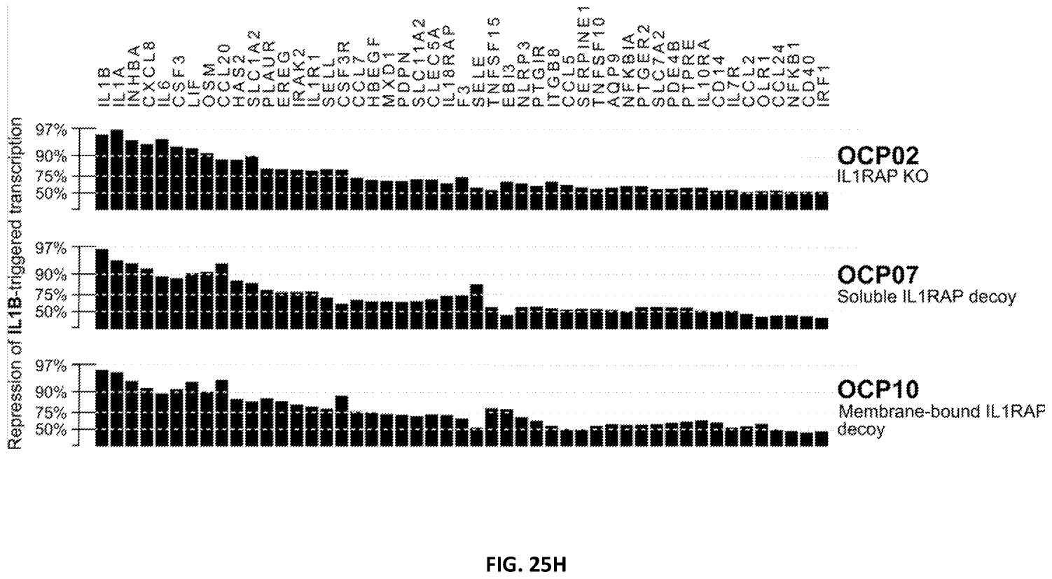

Functional assays had shown that IL1RAP knockouts or decoys made cells less responsive to some inflammatory triggers. However, how IL1 signaling contributes to the inflammatory response such as gout-like MSU crystals and whether the various strategies to interfere with it shows any differences on a genome-wide scale remained unclear. To test this, transcriptome-wide analysis was undertaken in either control or edited cells treated with various pro-inflammatory triggers for either 4 or 24 hours as previously described. The transcriptomes of these cells were then subjected to principal component analysis (PCA) to obtain a first view about their relationships to each other (FIG. 25A). This PCA (covering 77% of transcriptional variance across all samples) found that IL1RAP knockout and decoys were similarly effective in preventing differential transcription in cells treated with IL1β (both after 4 and 24 hrs) and MSU crystals (after 24 hrs), as noted by the distance between the control and IL1RAP-edited cells on the plot. However, LPS treatment was largely unaffected by the editing of IL1RAP, as noted by the clustering of control and edited cells on the plot. As expected, in the absence of an inflammatory trigger, genetic IL1RAP interference did not substantially change transcription as shown by the tight clustering of control and edited cells treated with PBS.

The results of the PCA were further supported by a more detailed analysis of differentially expressed genes. For instance, unedited cells exhibited strong transcriptional induction of numerous inflammatory markers upon stimulation with IL1β for 24 hours, including IL1B, IL6, PTGS2, and TNF (FIG. 25B). Similar results were observed with MSU treatment (FIG. 25C). However, cells with edited IL1RAP genes exhibited dramatically reduced induction of each of these genes. Notably, this reduction occurred regardless of the type of interference to IL1RAP (i.e., knockout or decoy receptor), and this reduction did not impact induction of the IL1R antagonist (IL1R2) or the housekeeping gene GAPDH.

A Venn diagram was generated for differentially expressed genes (fold change >2; FDR<10%) to show uniquely upregulated or downregulated in response to 24 hours of IL1B or MSU treatment in control and IL1RAP-edited cells. More than half of the surveyed transcripts-733 in total-were upregulated by IL1β treatment in unedited cells only (FIG. 25D). By contrast, no more than 72 upregulated genes were unique to each IL1RAP-edited cell pool. Similarly for non-shared downregulated genes, unedited cells showed more than half of the surveyed transcripts, while IL1RAP-edited cells were largely inert (FIG. 25E). A similar profile, albeit with less robust up- and downregulation of genes was observed with MSU treatment (FIGS. 25F, G). Taken together, these results demonstrated that (1) IL1RAP edits were the root cause of the functional differences observed between edited and control cells, (2) all types of gene interference demonstrated similar transcriptional profiles in being refractory to IL1β treatment, and (3) that IL1RAP interference could mitigate the inflammatory response to MSU crystals, which indicates a promising avenue to treat gout.

### Example 11: Validation of TGFBR1 Editing in Human Cells

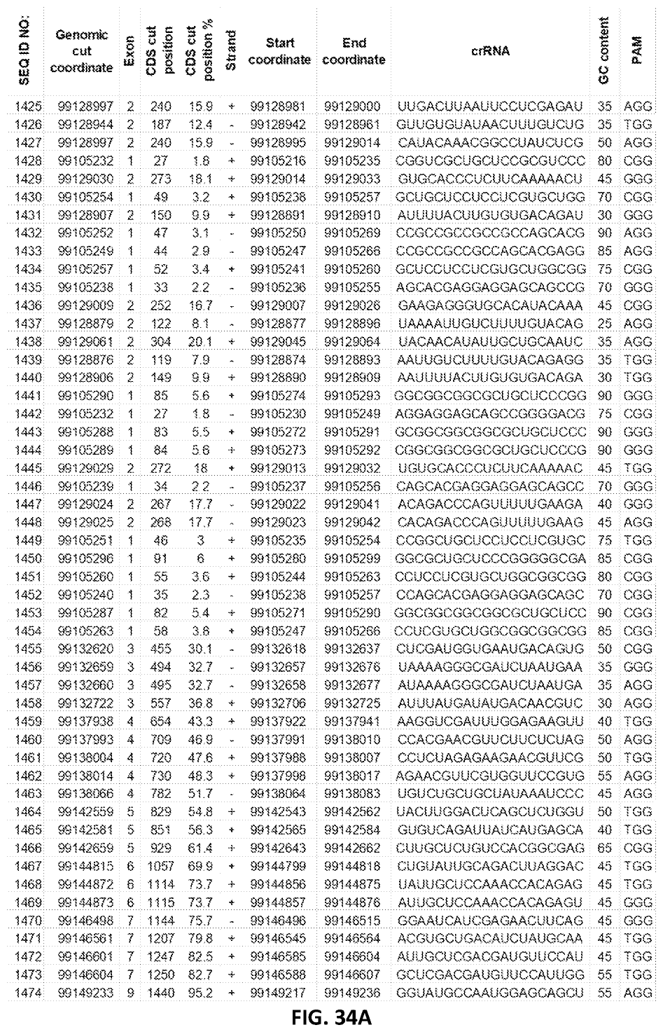

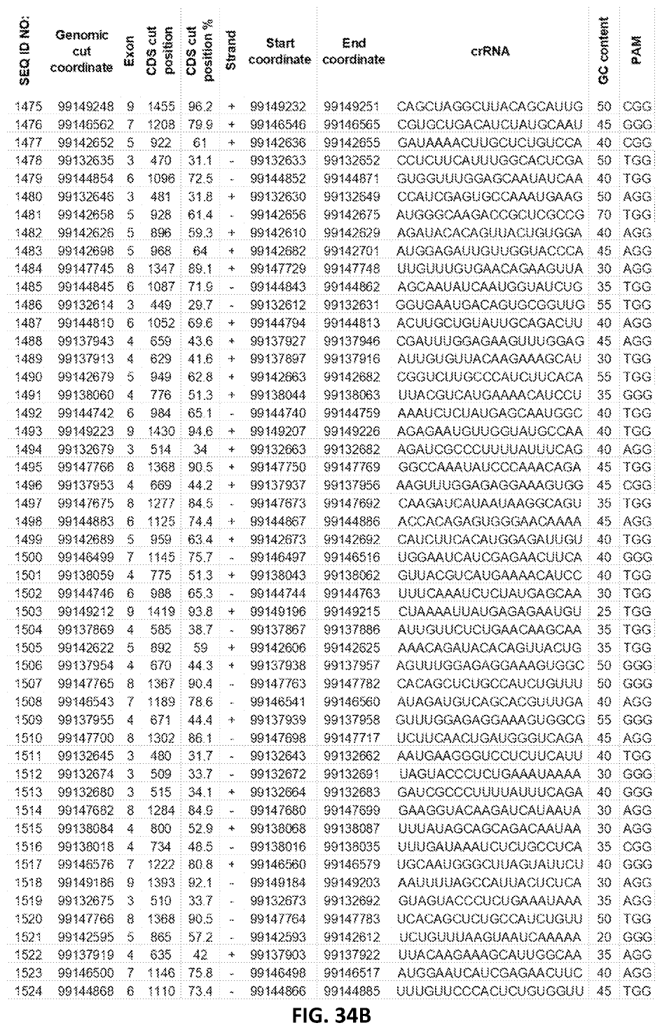

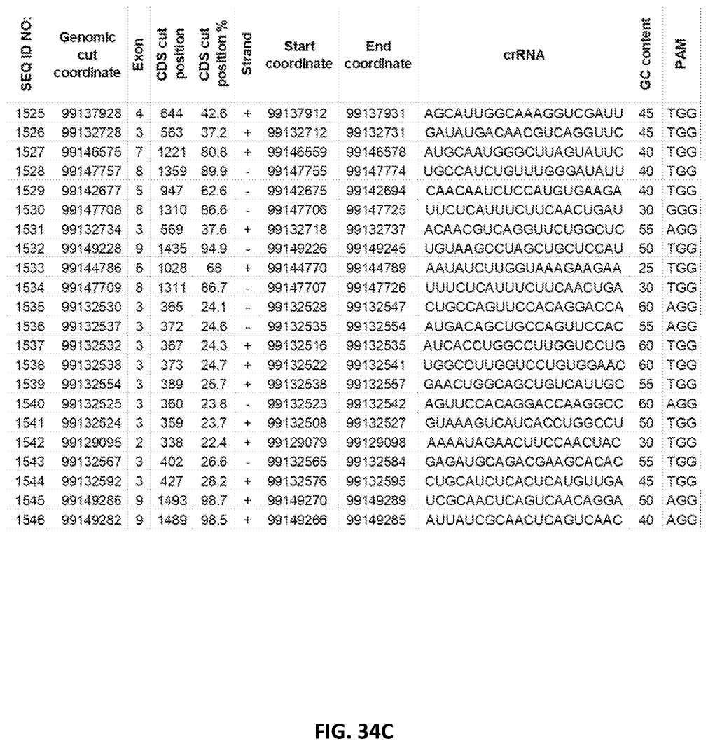

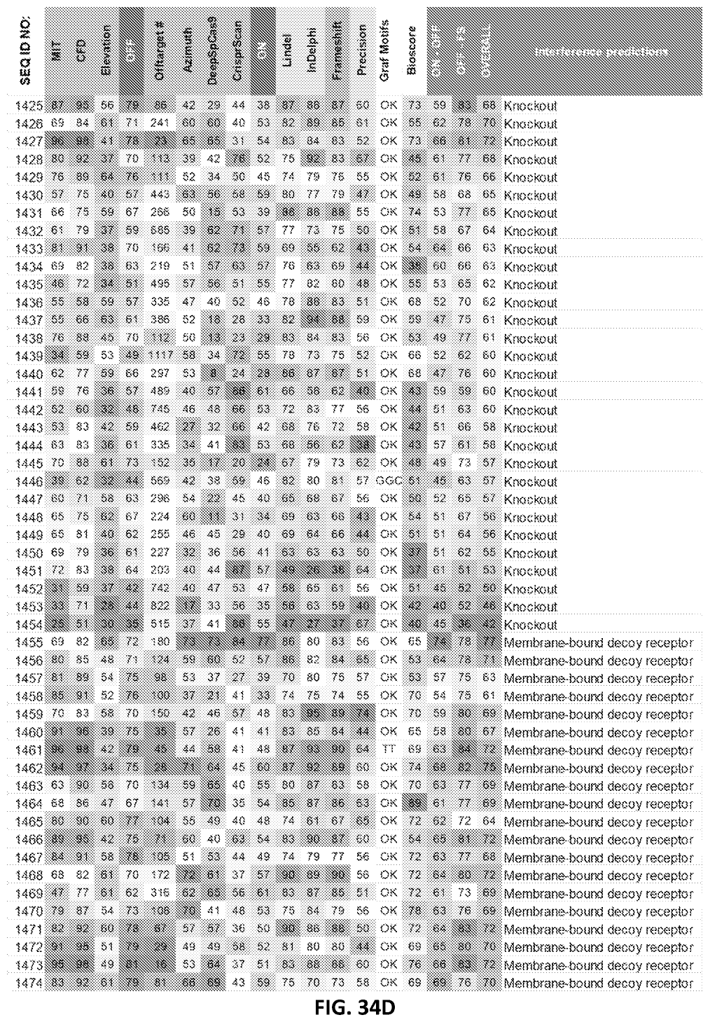

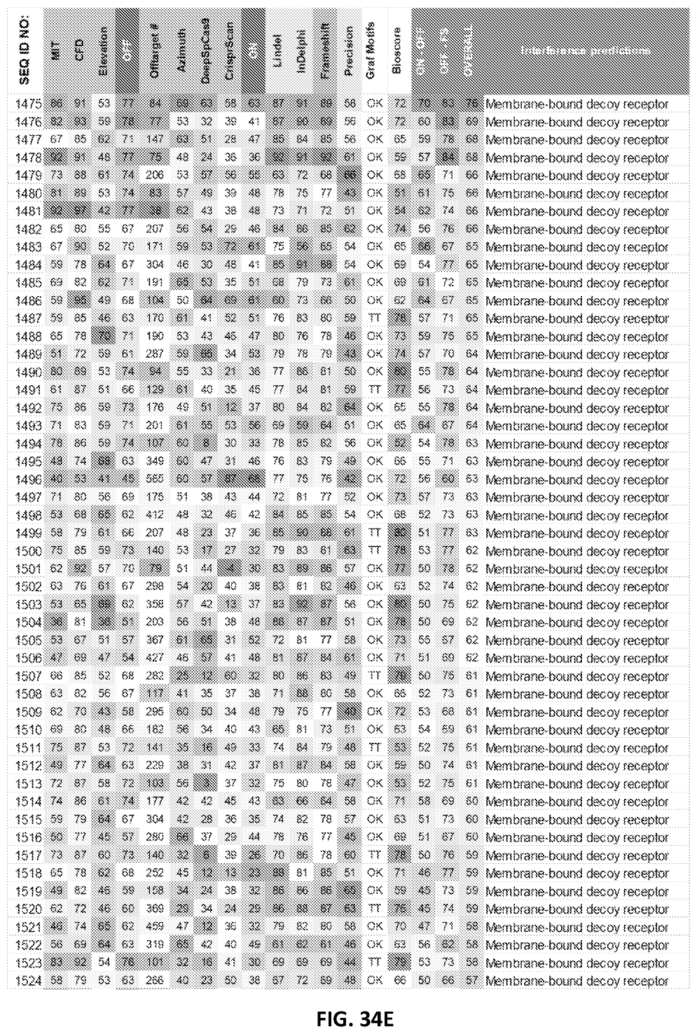

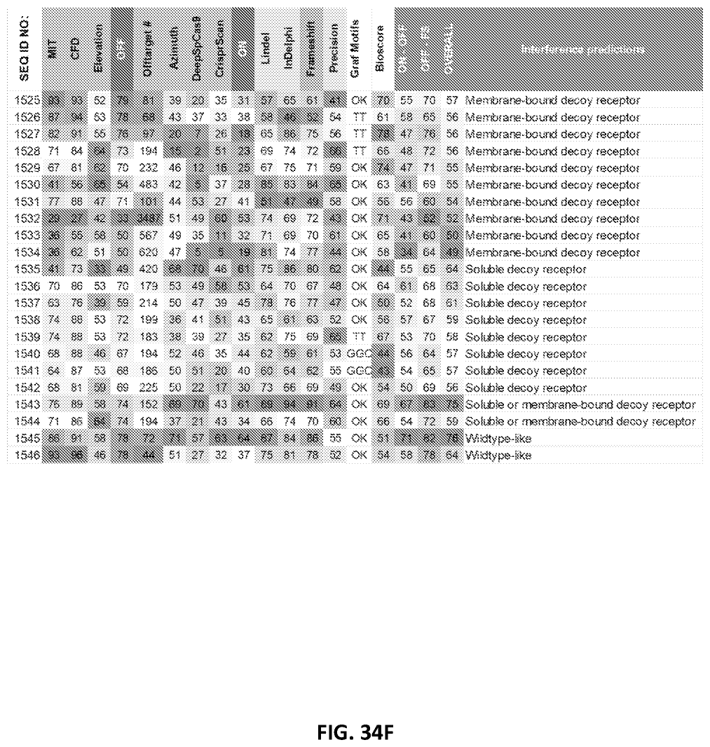

Having designed and analyzed numerous sgRNAs that were predicted to edit the TGFBR1 receptor to produce either a knockout or (soluble or membrane-bound) dominant negative decoy by bioinformatic methods described in Example 1 (see, FIG. 34), the next step was to assess their ability to act in vitro. Of particular interest was whether the one preferred, on-target edit would be observed in any of the candidates.

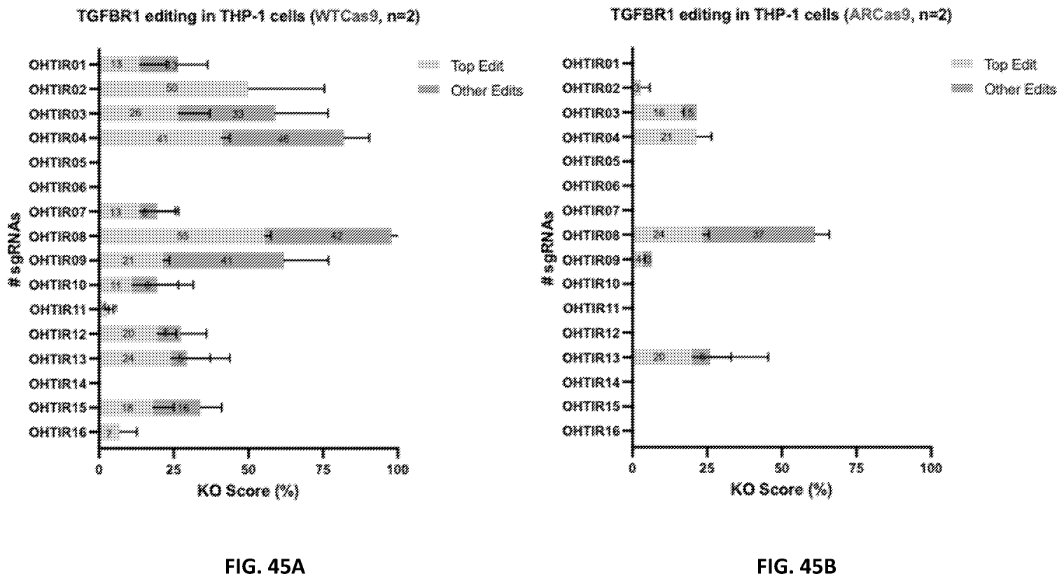

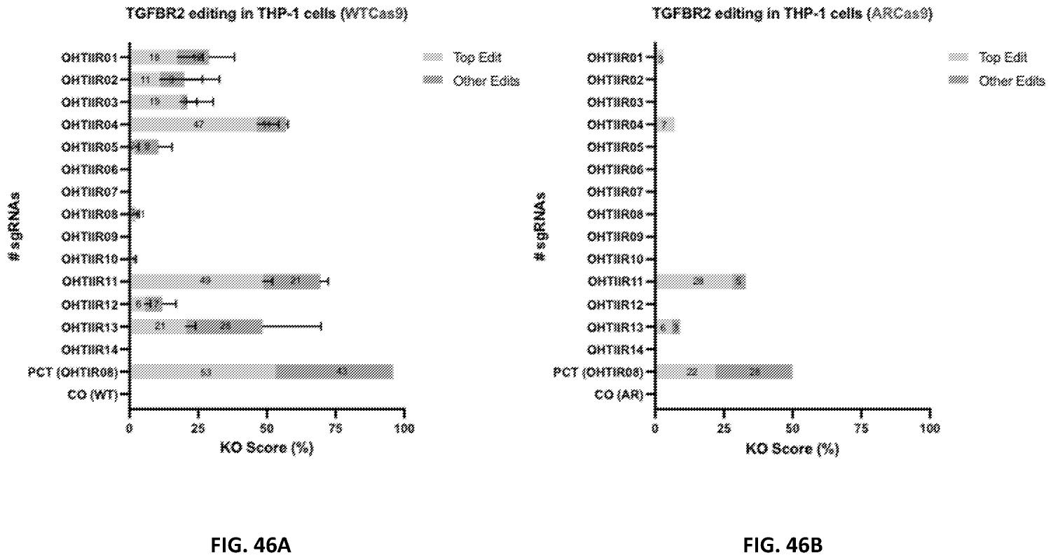

To test this, the sgRNA candidates were complexed with a Cas9 nuclease to form a ribonucleoprotein (RNP) complex before electroporating human THP-1 monocytes to introduce the RNPs to the cells as previously described above. Editing efficiency was then measured via sequencing. While multiple candidates were able to induce mutations in the hTGFBR1 gene at high efficiency, with several exhibiting frequencies above 50% with wtCas9 (FIG. 45A), this efficacy was universally reduced with use of the AR-Cas9 variant (FIG. 46B). Notably, pairing AR-Cas9 with OHTIR04 (SEQ ID NO: 1455) resulted in greater edit primacy (i.e., only the single, on-target edit was observed), albeit at a lower editing efficiency. These results generally confirmed the in silico analysis and suggested that several sgRNA candidates, including OHTIR04, were able to efficiently edit TGFBR1 in human cells at some frequency.

### Example 12: Validation of TGFBR2 Editing in Human Cells

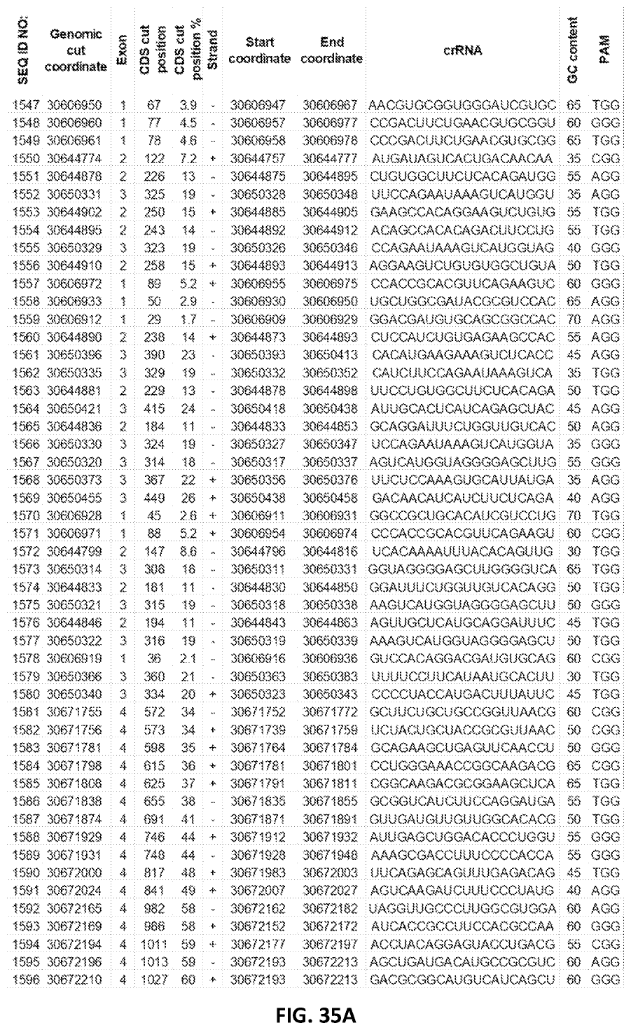

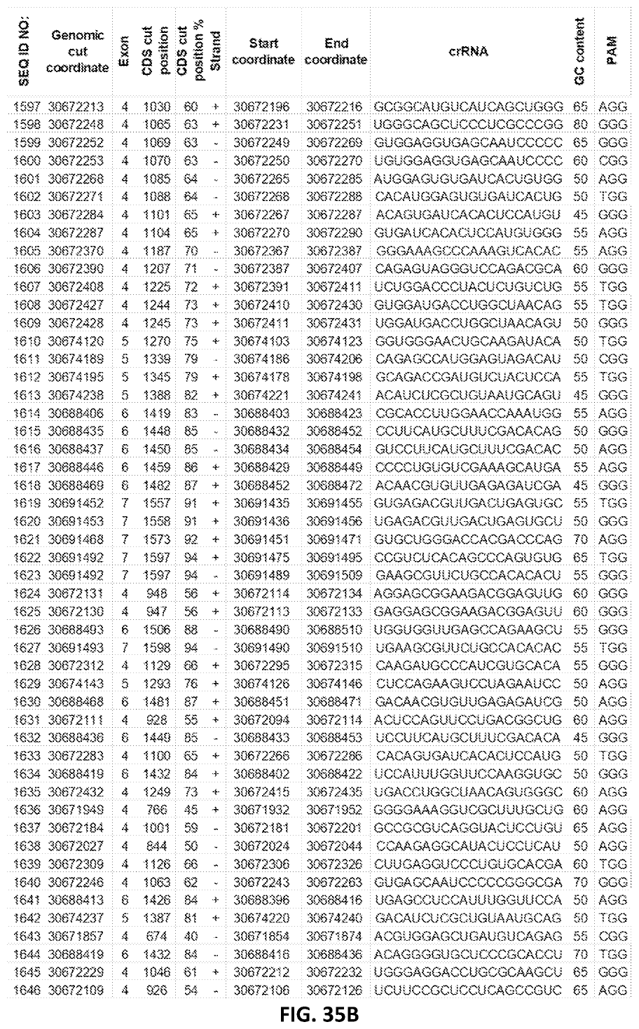

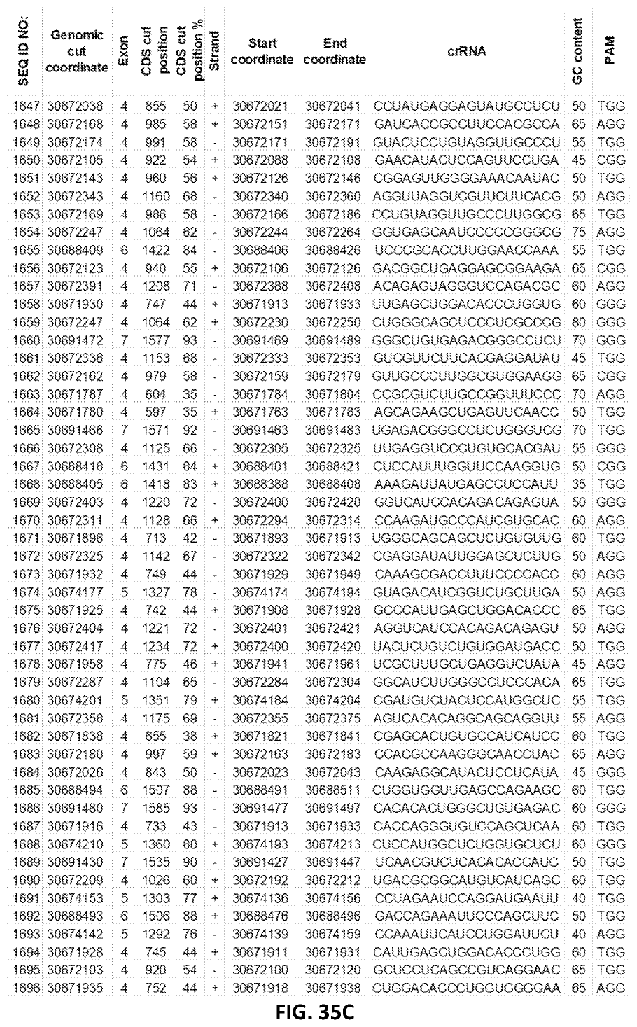

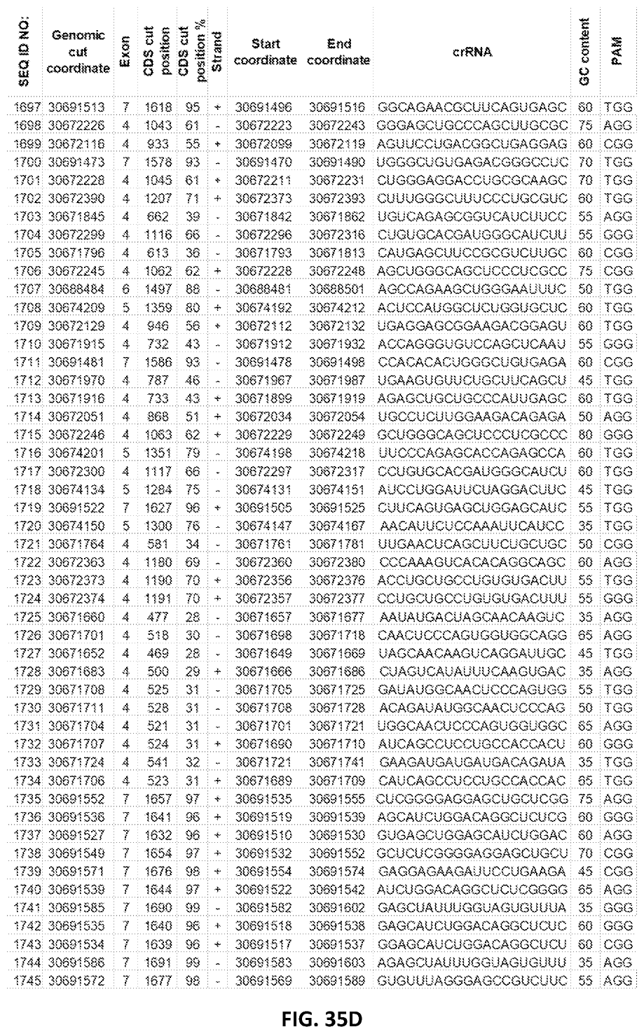

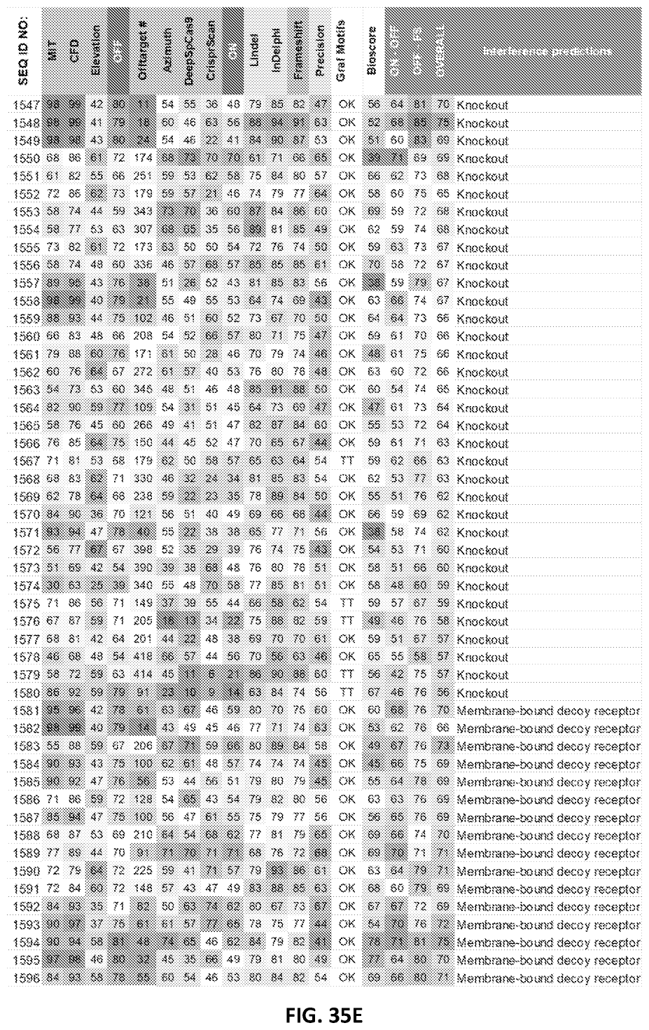

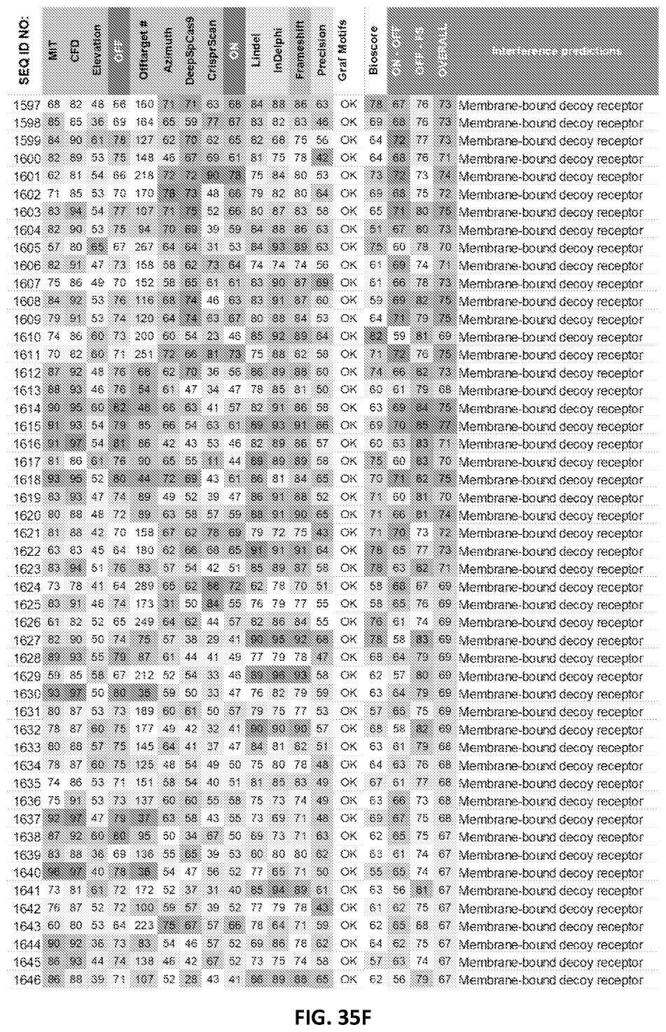

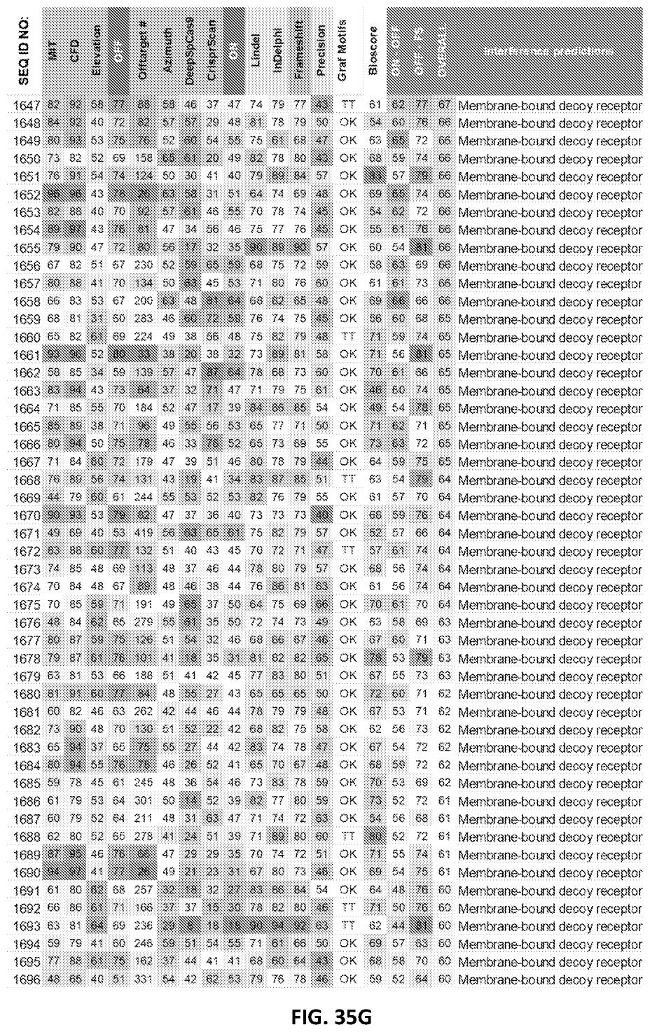

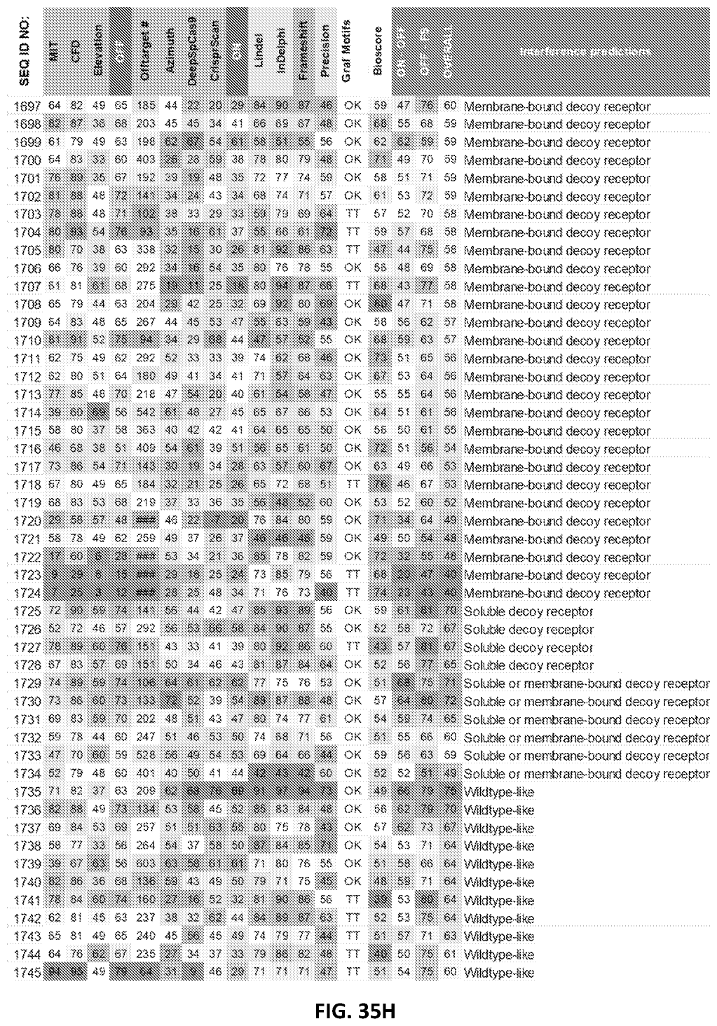

Having designed and analyzed numerous sgRNAs that were predicted to edit the TGFBR2 receptor to produce either a knockout or (soluble or membrane-bound) dominant negative decoy by bioinformatic methods described in Example 1 (see, FIG. 35), the next step was to assess their ability to act in vitro. Of particular interest was whether the one preferred edit would be observed in any of the candidates.

To test this, the sgRNA candidates were complexed with a Cas9 nuclease to form a ribonucleoprotein (RNP) complex before electroporating human THP-1 monocytes to introduce the RNPs to the cells as previously described above. Editing efficiency was then measured via sequencing. While multiple candidates were able to induce mutations in the hTGFBR2 gene at high efficiency, with several exhibiting frequencies near or above 50% with wtCas9 (FIG. 46A), this efficacy was universally reduced with use of the AR-Cas9 variant (FIG. 46B). Notably, pairing AR-Cas9 with OHTIIR11 (SEQ ID NO: 1466) resulted in increased editing primacy (i.e., proportion of the editing comprised by the desired on-target edit), albeit at a lower editing efficiency than with wtCas9. These results generally confirmed the in silico analysis and suggested that several sgRNA candidates, including OHTIIR11, were able to efficiently edit TGFBR2 in human cells at some frequency.

### Example 13: Editing of TGFBR1 or TGFBR2 Renders Human Monocytes Unresponsive to TGF-Beta Stimuli

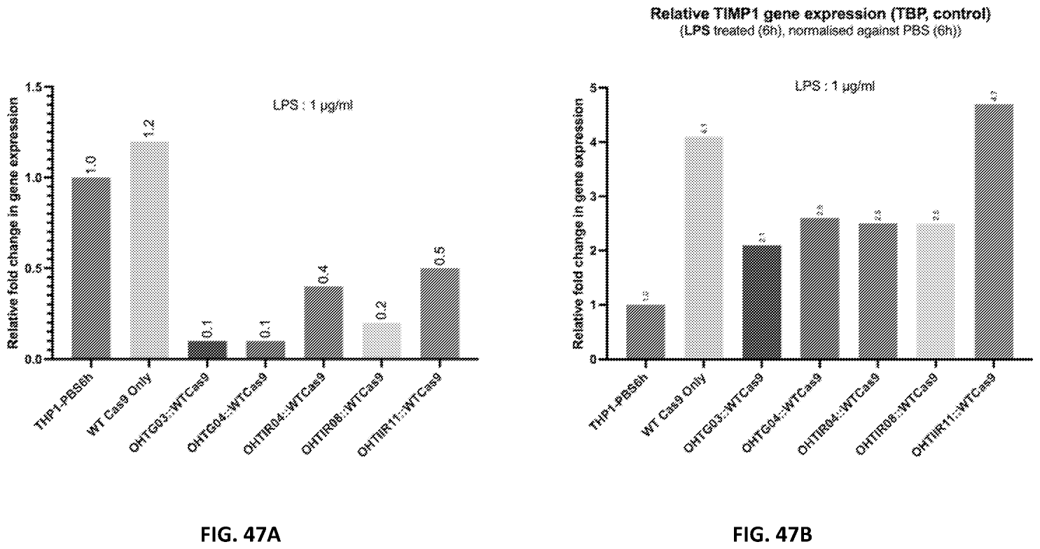

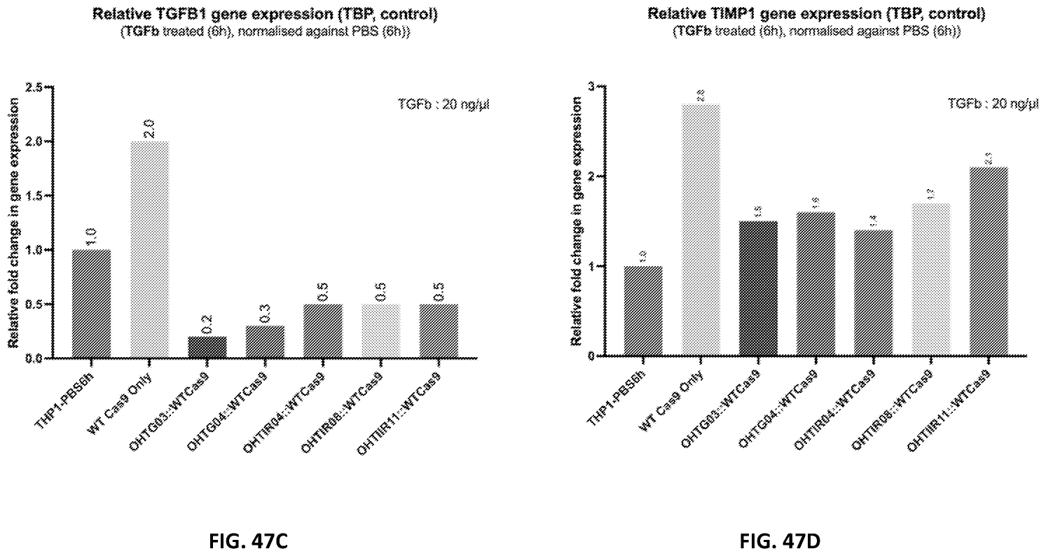

Having designed and validated numerous guides targeting the TGF-beta pathway, the next step involved functional testing of edited cells. As part of this testing, around 300,000 edited human THP-1 monocytes were treated with either 1 μg/ml LPS or 20 ng/μl recombinant TGF-beta for 6 hours. Reverse transcription of total cellular RNA was followed by quantitative PCR (RT-qPCR) was carried out to verify whether human either LPS or TGF-beta can stimulate the transcription of TGFB1 as has been previously reported (see generally, Yan, X., et al. (2018). Acta Biochimica et Biophysica Sinica, 50 (1), 37-50.). LPS treatment did not induce robust expression of TGFB1 in control cells as compared to those treated with PBS (FIG. 47A). Despite this lack of response, TGFB1 levels were reduced as much as five-fold for TGFBR1-edited cells compared to the unedited control, while TGFBR2-edited cells exhibited a more modest two-fold reduction.

As an additional test, the same cellular RNA was assayed for induction of TIMP1, which canonically is upregulated by TGF-beta signaling via the SMAD proteins (see generally, Hall, M. C., et al. (2003). Journal of Biological Chemistry, 278 (12), 10304-10313.). In the LPS-treated cells, robust induction of TIMP1 was observed in the unedited control as compared to the PBS-treated cells (FIG. 47B). TGFBR1-edited monocytes demonstrated a two-fold decrease in TIMP1 induction as compared to the unedited control. There was no change for TGFBR2-edited cells

Results were even more striking following treatment with the natural ligand. In response to TGF-beta treatment, TGFB1 induction was more robust, and edits to either TGFBR1 or TGFBR2 reduced this response by two-fold (FIG. 47C). These results were similar to those observed for TIMP1 induction following TGF-beta treatment (FIG. 47D). These results collectively showed that the edited cells did not respond to TGF-beta treatment, as their levels of TGFB1 remained well below the PBS-treated control. Taken together, these results demonstrated that TGF-beta positive feedback (TGFB1 induction) and downstream signaling (TIMP1 induction) were both impaired in cells with edited TGFBR1 and TGFBR2.

### Example 14: Validation of IL1R1 Editing in Human Cells

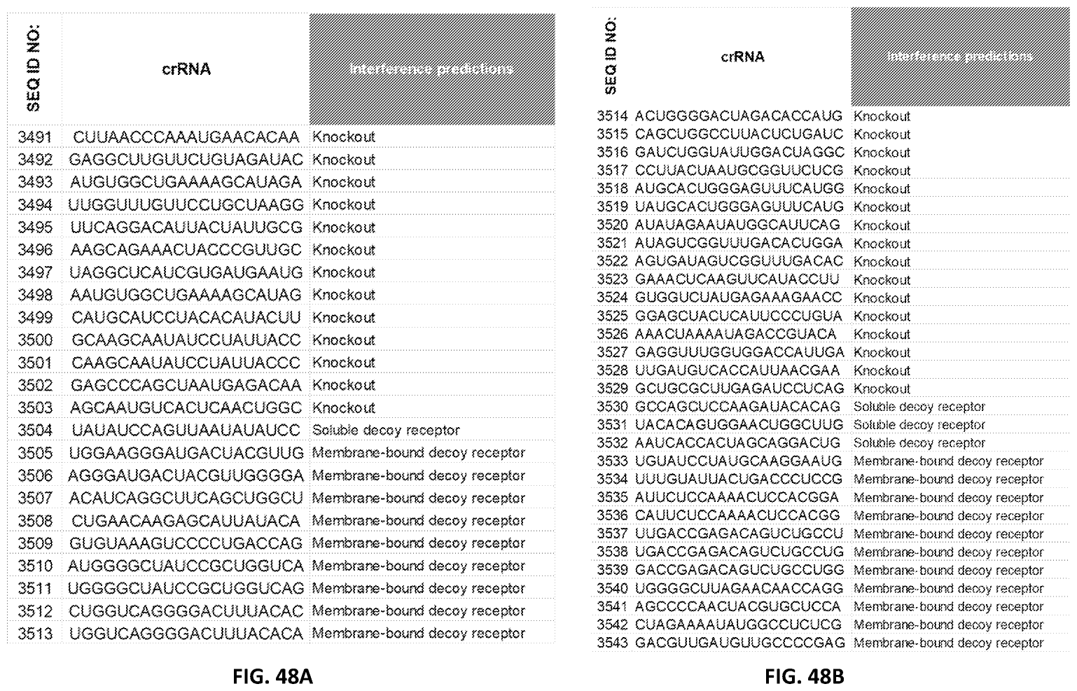

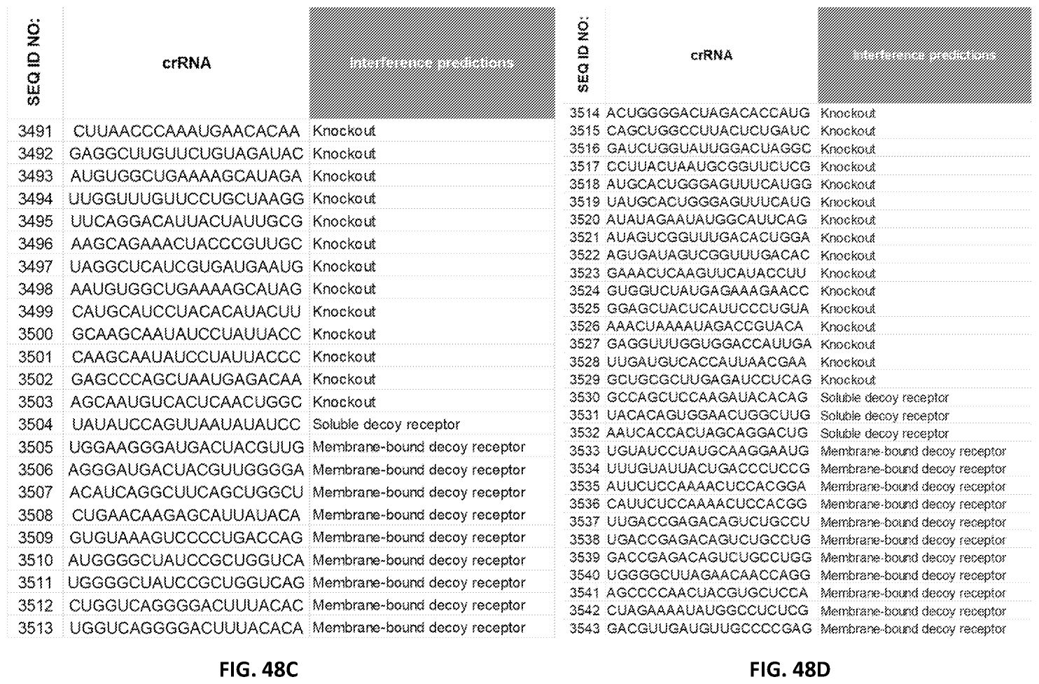

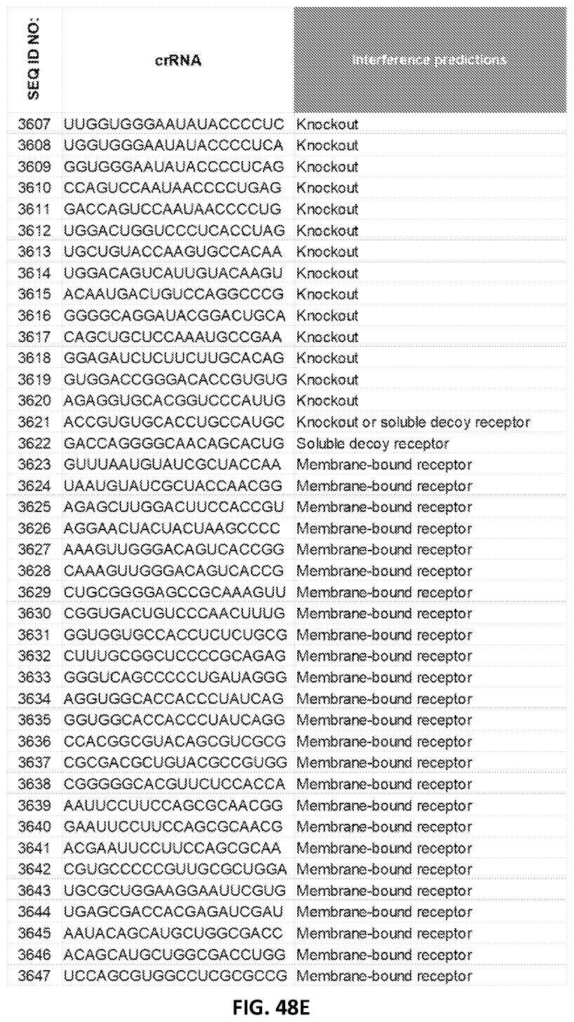

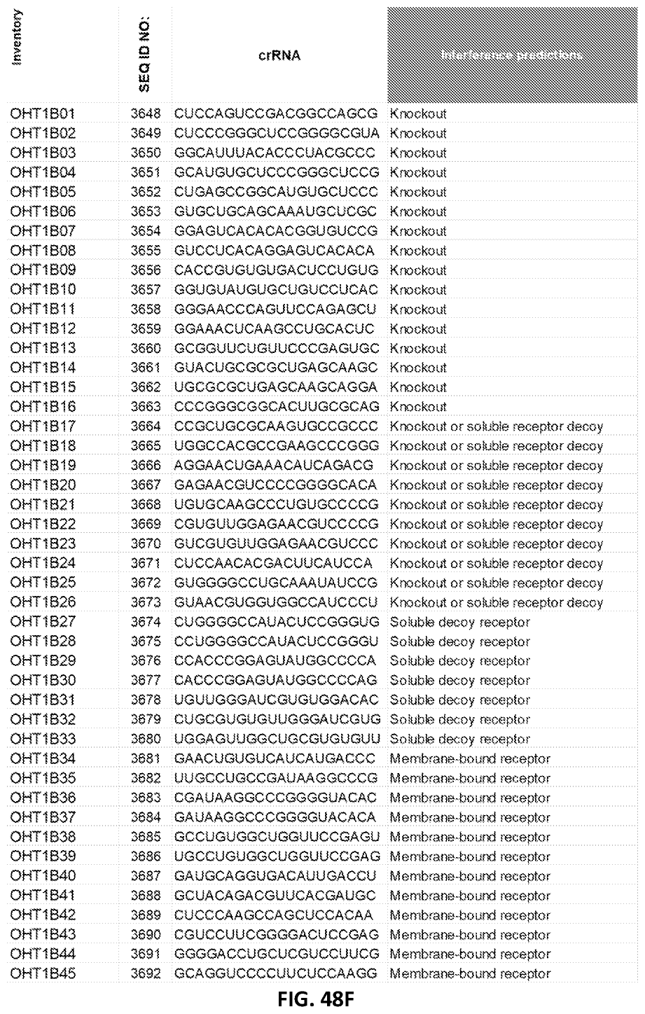

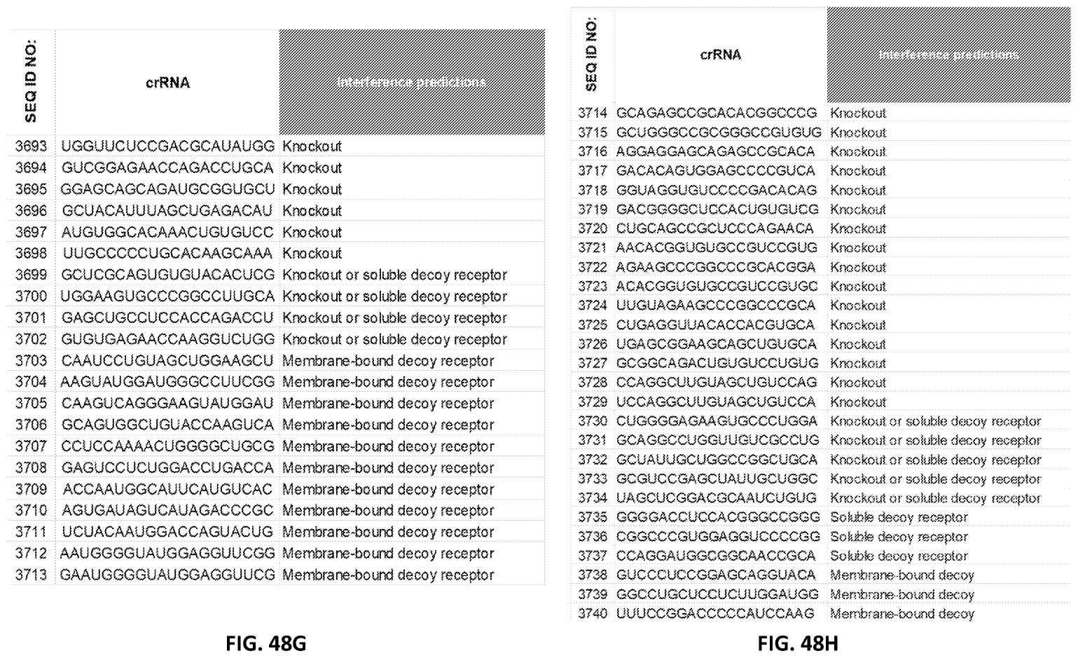

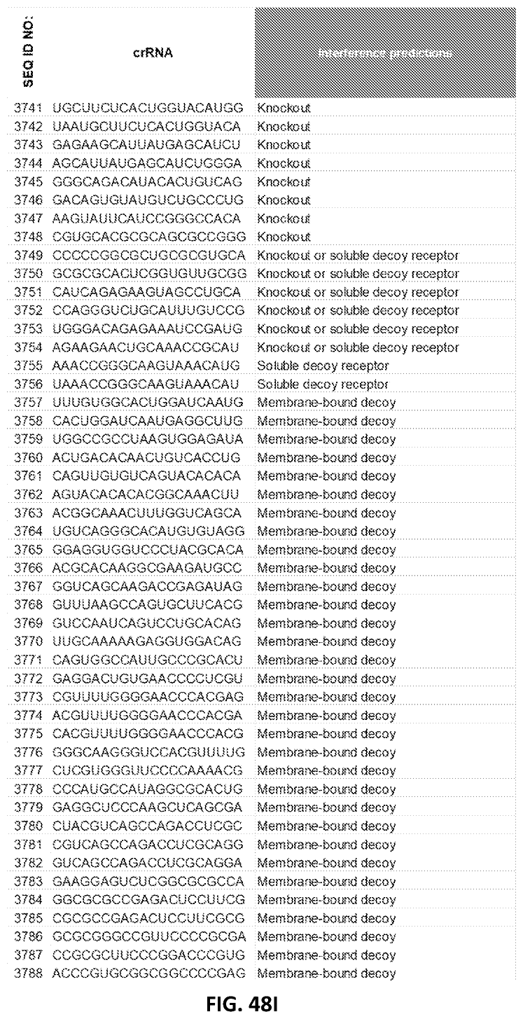

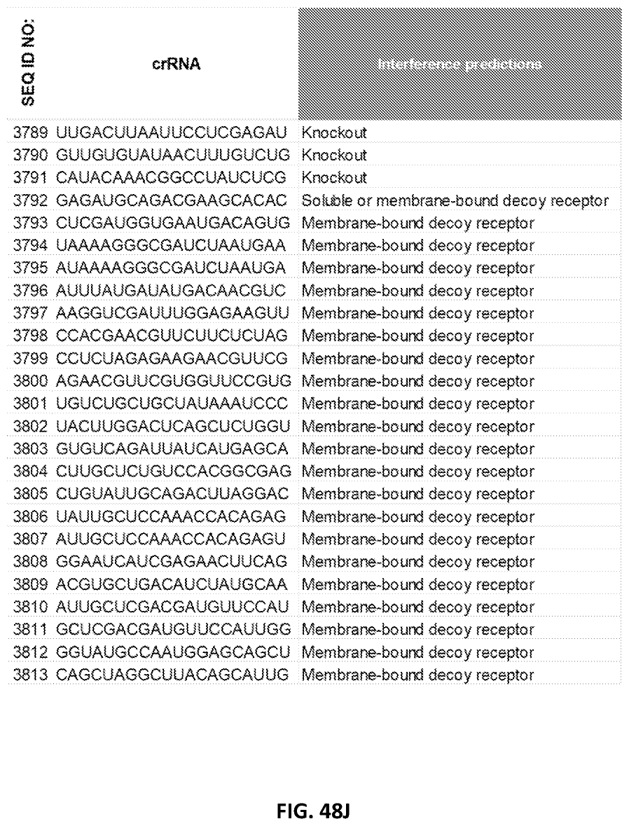

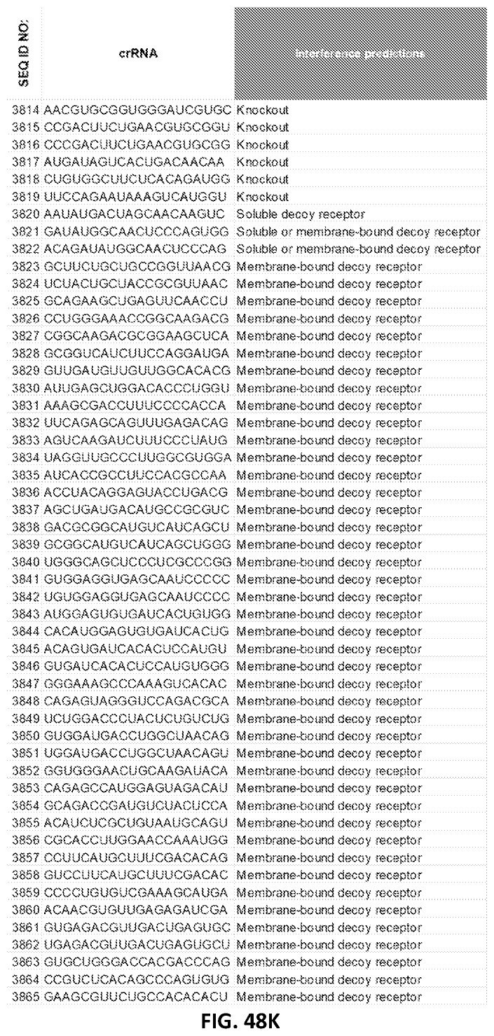

Having designed and analyzed numerous sgRNA candidates that were predicted to edit the human IL1 receptor to produce either a knockout or a dominant negative receptor (see FIG. 48A), the next step is to assess their ability to act in vitro. Of particular interest is whether the preferred (i.e., on-target) edit will be observed in edited cells.

To test this, 80 pmoles of sgRNA candidates (SEQ ID NOs: 3491-3513) are introduced into human THP-1 monocytes via electroporation as part of a ribonucleoprotein (i.e., pre-assembled with 25 pmoles or either wtCas9 or AR-Cas9 protein at room temperature for at least 5 min). In brief, around 600,000 cells resuspended in 20 μl PKM buffer (2.7 mM KCl, 137 mM NaCl, 10 mM Na2HPO4/1.8 mM NaH2PO4 (pH 7.4) and 50 mM D-Mannitol) are added to the pre-assembled Cas9 ribonucleoprotein complex, transferred into 16-well nucleocuvettes and electroporated with pulse code CM-137 using the 4D nucleofector (Lonza). Electroporated cells are kept in PKM buffer for about 10 min before transferring them into 6-well tissue culture plates with complete culture media. After 2-5 days in culture, cells are dissociated for genomic DNA extraction using the DNeasy Blood & Tissue Kit (Qiagen). sgRNA target regions are amplified by PCR and editing efficacy is then deduced from Sanger sequencing traces using Inference of CRISPR Edits (ICE v1.2). Conant, D. et al. (2022). The CRISPR Journal, 5 (1), 123-130 (2022).

It is anticipated that results of these studies will confirm the robustness of the in silico analysis and identify multiple candidates are indeed able to induce mutation in the IL1R1 gene at high efficacy. It is further expected that use of AR-Cas9 will improve editing efficacy (i.e., increase on-target editing) as compared to wtCas9.

### Example 15: In Vitro Delivery of IL1R1-Editing Guide RNA to Human Cells Via Lipid Nanoparticles

Because all validation experiments are to be conducted via electroporation of cell cultures to introduce RNA-dependent nucleases and sgRNAs, additional experiments will assess the suitability of using lipid nanoparticle (LNP) to deliver mRNA to human cells. 0.025 mg/mL of LNPs from PNI containing either 80 pmoles of sgRNA (SEQ ID NOs: 3491-3513) or 80 pmoles of Cas9 mRNA (alternatively, sgRNA may be pre-assembled with 25 pmoles or either wtCas9 or AR-Cas9 protein at room temperature for at least 5 min and encapsulated as an RNP) are exposed to THP-1 monocytes for 8 to 24 hours under typical cell culture conditions. Sanger sequencing as described in Example 14 will be used to validate editing. Upon confirmation of editing various functional assays as described in Example 13 will be performed.

### Example 16: In Vitro Delivery of IL1R1-Editing Guide RNA to Human Cells Via Adeno-Associated Virus

Because all validation experiments are to be conducted via electroporation of cell cultures to introduce RNA-dependent nucleases and sgRNAs, additional experiments will assess the suitability of using adeno-associated virus (AAV) vectors to deliver sgRNA to human cells.

TABLE 2
Exemplary AAV vectors for introduction of sgRNA to human cells.

AAVPrime ™ Adeno-Associated Virus - Serotype6 (AAV-6) Particles for sgRNA
(GeneCopoeia ™, catalog No. AA06-MCP001682-AD01-2-200-a, lot No. GC03182K2001)
AAVPrime ™ Adeno-Associated Virus - Serotype6 (AAV-6) Particles for sgRNA
(GeneCopoeia ™, catalog No. AA06-MCP001682-AD01-2-200-b, lot No. GC03202K2001)
AAVPrime ™ Adeno-Associated Virus - Serotype6 (AAV-6) Particles for sgRNA
(GeneCopoeia ™, catalog No. AA06-CCPCTR01-AD01-200, lot No. GC03112K2002)
AAVPrime ™ Adeno-Associated Virus - Serotype5 (AAV-5) Particles for sgRNA
(GeneCopoeia ™, catalogue No. AA05-MCP001682-AD01-2-200A, lot No. GC-
03182K2002)
AAVPrime ™ Adeno-Associated Virus - Serotype5 (AAV-5) Particles for sgRNA
(GeneCopoeia ™, catalogue No. AA05-MCP001682-AD01-2-200B, lot No.
GC03182K2003)
AAVPrime ™ Adeno-Associated Virus - Serotype5 (AAV-5) Particles for sgRNA
(GeneCopoeia ™, catalogue No. AA05-CCPCTR01-AD01-200, lot No. GC03032K2003)

Exemplary AAV vectors are shown in Table 2. After being prepared according to manufacturer specifications, such that one or more of the sgRNAs containing SEQ ID NOs: 3491-3513 are integrated into the viral vector, said vectors are incubated (MOI=0.1 to 10) with approximately 500,000 U2OS cells that have been stably transfected with Cas9 (see, e.g., Rojas-Fernandez, A., et al. (2015). Scientific Reports, 5 (1), 1-6.). At 2-7 days post-infection, Sanger sequencing as described in Example 14 will be used to validate editing. Upon confirmation of editing, various functional assays as described in Example 13 will be performed.

### Example 17: Validation of IL1Rap Editing in Human Cells

Having designed and analyzed numerous sgRNA candidates that were predicted to edit the human IL1 receptor accessory protein to produce either a knockout or a dominant negative receptor (see FIG. 48B), the next step is to assess their ability to act in vitro. Of particular interest is whether the preferred (i.e., on-target) edit will be observed in edited cells.

To test this, 80 pmoles of sgRNA candidates (SEQ ID NOs: 3514-3543) are introduced into human THP-1 monocytes via electroporation as part of a ribonucleoprotein as previously described in Example 14.

It is anticipated that results of these studies will confirm the robustness of the in silico analysis and identify multiple candidates are indeed able to induce mutation in the IL1RAP gene at high efficacy. It is further expected that use of AR-Cas9 will improve editing efficacy (i.e., increase on-target editing) as compared to wtCas9.

### Example 18: In Vitro Delivery of IL1Rap-Editing Guide RNA to Human Cells Via Lipid Nanoparticles

Because all validation experiments are to be conducted via electroporation of cell cultures to introduce RNA-dependent nucleases and sgRNAs, additional experiments will assess the suitability of using lipid nanoparticle (LNP) to deliver mRNA to human cells. LNPs containing either sgRNA (SEQ ID NOs: 3514-3543) or Cas9 mRNA are incubated with THP-1 monocytes, followed by validation of editing as described in Example 15. Upon confirmation of editing various functional assays as described in Example 13 will be performed.

### Example 19: In Vitro Delivery of IL1Rap-Editing Guide RNA to Human Cells Via Adeno-Associated Virus

Because all validation experiments are to be conducted via electroporation of cell cultures to introduce RNA-dependent nucleases and sgRNAs, additional experiments will assess the suitability of using adeno-associated virus (AAV) vectors to deliver sgRNA to human cells. Exemplary AAV vectors are shown in Table 2. After being prepared according to manufacturer specifications, such that one or more of the sgRNAs containing SEQ ID NOs: 3514-3543 are integrated into the viral vector, said vectors are introduced to human U2OS cells and editing is validated as previously described in Example 16. Upon confirmation of editing, various functional assays as described in Example 13 will be performed.

### Example 20: Validation of IL6R Editing in Human Cells

Having designed and analyzed numerous sgRNA candidates that were predicted to edit the human IL6 receptor protein to produce either a knockout or a dominant negative receptor (see FIG. 48C), the next step is to assess their ability to act in vitro. Of particular interest is whether the preferred (i.e., on-target) edit will be observed in edited cells.

To test this, 80 pmoles of sgRNA candidates (SEQ ID NOs: 3544-3566) are introduced into human THP-1 monocytes via electroporation as part of a ribonucleoprotein as previously described in Example 14.

It is anticipated that results of these studies will confirm the robustness of the in silico analysis and identify multiple candidates are indeed able to induce mutation in the IL6R gene at high efficacy. It is further expected that use of AR-Cas9 will improve editing efficacy (i.e., increase on-target editing) as compared to wtCas9.

### Example 21: In Vitro Delivery of IL6R-Editing Guide RNA to Human Cells Via Lipid Nanoparticles

Because all validation experiments are to be conducted via electroporation of cell cultures to introduce RNA-dependent nucleases and sgRNAs, additional experiments will assess the suitability of using lipid nanoparticle (LNP) to deliver mRNA to human cells. LNPs containing either sgRNA (SEQ ID NOs: 3544-3566) or Cas9 mRNA are incubated with THP-1 monocytes, followed by validation of editing as described in Example 15. Upon confirmation of editing various functional assays as described in Example 13 will be performed.

### Example 22: In Vitro Delivery of IL6R-Editing Guide RNA to Human Cells Via Adeno-Associated Virus

Because all validation experiments are to be conducted via electroporation of cell cultures to introduce RNA-dependent nucleases and sgRNAs, additional experiments will assess the suitability of using adeno-associated virus (AAV) vectors to deliver sgRNA to human cells. Exemplary AAV vectors are shown in Table 2. After being prepared according to manufacturer specifications, such that one or more of the sgRNAs containing SEQ ID NOs: 3544-3566 are integrated into the viral vector, said vectors are introduced to human U2OS cells and editing is validated as previously described in Example 16. Upon confirmation of editing, various functional assays as described in Example 13 will be performed.

### Example 23: Validation of IL6ST Editing in Human Cells

Having designed and analyzed numerous sgRNA candidates that were predicted to edit the human IL6 Cytokine Family Signal Transducer protein to produce either a knockout or a dominant negative receptor (see FIG. 48D), the next step is to assess their ability to act in vitro. Of particular interest is whether the preferred (i.e., on-target) edit will be observed in edited cells.

To test this, 80 pmoles of sgRNA candidates (SEQ ID NOs: 3567-3606) are introduced into human THP-1 monocytes via electroporation as part of a ribonucleoprotein as previously described in Example 14.

It is anticipated that results of these studies will confirm the robustness of the in silico analysis and identify multiple candidates are indeed able to induce mutation in the IL6ST gene at high efficacy. It is further expected that use of AR-Cas9 will improve editing efficacy (i.e., increase on-target editing) as compared to wtCas9.

### Example 24: In Vitro Delivery of IL6ST-Editing Guide RNA to Human Cells Via Lipid Nanoparticles

Because all validation experiments are to be conducted via electroporation of cell cultures to introduce RNA-dependent nucleases and sgRNAs, additional experiments will assess the suitability of using lipid nanoparticle (LNP) to deliver mRNA to human cells. LNPs containing either sgRNA (SEQ ID NOs: 3567-3606) or Cas9 mRNA are incubated with THP-1 monocytes, followed by validation of editing as described in Example 15. Upon confirmation of editing various functional assays as described in Example 13 will be performed.

### Example 25: In Vitro Delivery of IL6ST-Editing Guide RNA to Human Cells Via Adeno-Associated Virus

Because all validation experiments are to be conducted via electroporation of cell cultures to introduce RNA-dependent nucleases and sgRNAs, additional experiments will assess the suitability of using adeno-associated virus (AAV) vectors to deliver sgRNA to human cells. Exemplary AAV vectors are shown in Table 2. After being prepared according to manufacturer specifications, such that one or more of the sgRNAs containing SEQ ID NOs: 3567-3606 are integrated into the viral vector, said vectors are introduced to human U20S cells and editing is validated as previously described in Example 16. Upon confirmation of editing, various functional assays as described in Example 13 will be performed.

### Example 26: Validation of TNFRSF1A Editing in Human Cells

Having designed and analyzed numerous sgRNA candidates that were predicted to edit the human Tumor Necrosis Factor Receptor 1 protein to produce either a knockout or a dominant negative receptor (see FIG. 48E), the next step is to assess their ability to act in vitro. Of particular interest is whether the preferred (i.e., on-target) edit will be observed in edited cells.

To test this, 80 pmoles of sgRNA candidates (SEQ ID NOs: 3607-3647) are introduced into human THP-1 monocytes via electroporation as part of a ribonucleoprotein as previously described in Example 14.

It is anticipated that results of these studies will confirm the robustness of the in silico analysis and identify multiple candidates are indeed able to induce mutation in the TNFRSF1A gene at high efficacy. It is further expected that use of AR-Cas9 will improve editing efficacy (i.e., increase on-target editing) as compared to wtCas9.

### Example 27: In Vitro Delivery of TNFRSF1A-Editing Guide RNA to Human Cells Via Lipid Nanoparticles

Because all validation experiments are to be conducted via electroporation of cell cultures to introduce RNA-dependent nucleases and sgRNAs, additional experiments will assess the suitability of using lipid nanoparticle (LNP) to deliver mRNA to human cells. LNPs containing either sgRNA (SEQ ID NOs: 3607-3647) or Cas9 mRNA are incubated with THP-1 monocytes, followed by validation of editing as described in Example 15. Upon confirmation of editing various functional assays as described in Example 13 will be performed.

### Example 28: In Vitro Delivery of TNFRSF1A-Editing Guide RNA to Human Cells Via Adeno-Associated Virus

Because all validation experiments are to be conducted via electroporation of cell cultures to introduce RNA-dependent nucleases and sgRNAs, additional experiments will assess the suitability of using adeno-associated virus (AAV) vectors to deliver sgRNA to human cells. Exemplary AAV vectors are shown in Table 2. After being prepared according to manufacturer specifications, such that one or more of the sgRNAs containing SEQ ID NOs: 3607-3647 are integrated into the viral vector, said vectors are introduced to human U2OS cells and editing is validated as previously described in Example 16. Upon confirmation of editing, various functional assays as described in Example 13 will be performed.

### Example 29: Validation of TNFRSF1B Editing in Human Cells

Having designed and analyzed numerous sgRNA candidates that were predicted to edit the human Tumor Necrosis Factor Receptor 2 protein to produce either a knockout or a dominant negative receptor (see FIG. 48F), the next step is to assess their ability to act in vitro. Of particular interest is whether the preferred (i.e., on-target) edit will be observed in edited cells.

To test this, 80 pmoles of sgRNA candidates (SEQ ID NOs: 3648-3692) are introduced into human THP-1 monocytes via electroporation as part of a ribonucleoprotein as previously described in Example 14.

It is anticipated that results of these studies will confirm the robustness of the in silico analysis and identify multiple candidates are indeed able to induce mutation in the TNFRSF1B gene at high efficacy. It is further expected that use of AR-Cas9 will improve editing efficacy (i.e., increase on-target editing) as compared to wtCas9.

### Example 30: In Vitro Delivery of TNFRSF1B-Editing Guide RNA to Human Cells Via Lipid Nanoparticles

Because all validation experiments are to be conducted via electroporation of cell cultures to introduce RNA-dependent nucleases and sgRNAs, additional experiments will assess the suitability of using lipid nanoparticle (LNP) to deliver mRNA to human cells. LNPs containing either sgRNA (SEQ ID NOs: 3648-3692) or Cas9 mRNA are incubated with THP-1 monocytes, followed by validation of editing as described in Example 15. Upon confirmation of editing various functional assays as described in Example 13 will be performed.

### Example 31: In Vitro Delivery of TNFRSF1B-Editing Guide RNA to Human Cells Via Adeno-Associated Virus

Because all validation experiments are to be conducted via electroporation of cell cultures to introduce RNA-dependent nucleases and sgRNAs, additional experiments will assess the suitability of using adeno-associated virus (AAV) vectors to deliver sgRNA to human cells. Exemplary AAV vectors are shown in Table 2. After being prepared according to manufacturer specifications, such that one or more of the sgRNAs containing SEQ ID NOs: 3648-3692 are integrated into the viral vector, said vectors are introduced to human U2OS cells and editing is validated as previously described in Example 16. Upon confirmation of editing, various functional assays as described in Example 13 will be performed.

### Example 32: Validation of TNFRSF3 Editing in Human Cells

Having designed and analyzed numerous sgRNA candidates that were predicted to edit the human Lymphotoxin Beta Receptor protein to produce either a knockout or a dominant negative receptor (see FIG. 48G), the next step is to assess their ability to act in vitro. Of particular interest is whether the preferred (i.e., on-target) edit will be observed in edited cells.

To test this, 80 pmoles of sgRNA candidates (SEQ ID NOs: 3693-3713) are introduced into human THP-1 monocytes via electroporation as part of a ribonucleoprotein as previously described in Example 14.

It is anticipated that results of these studies will confirm the robustness of the in silico analysis and identify multiple candidates are indeed able to induce mutation in the TNFRSF3 gene at high efficacy. It is further expected that use of AR-Cas9 will improve editing efficacy (i.e., increase on-target editing) as compared to wtCas9.

### Example 33: In Vitro Delivery of TNFRSF3-Editing Guide RNA to Human Cells Via Lipid Nanoparticles

Because all validation experiments are to be conducted via electroporation of cell cultures to introduce RNA-dependent nucleases and sgRNAs, additional experiments will assess the suitability of using lipid nanoparticle (LNP) to deliver mRNA to human cells. LNPs containing either sgRNA (SEQ ID NOs: 3693-3713) or Cas9 mRNA are incubated with THP-1 monocytes, followed by validation of editing as described in Example 15. Upon confirmation of editing various functional assays as described in Example 13 will be performed.

### Example 34: In Vitro Delivery of TNFRSF3-Editing Guide RNA to Human Cells Via Adeno-Associated Virus

Because all validation experiments are to be conducted via electroporation of cell cultures to introduce RNA-dependent nucleases and sgRNAs, additional experiments will assess the suitability of using adeno-associated virus (AAV) vectors to deliver sgRNA to human cells. Exemplary AAV vectors are shown in Table 2. After being prepared according to manufacturer specifications, such that one or more of the sgRNAs containing SEQ ID NOs: 3693-3713 are integrated into the viral vector, said vectors are introduced to human U2OS cells and editing is validated as previously described in Example 16. Upon confirmation of editing, various functional assays as described in Example 13 will be performed.

### Example 35: Validation of TNFRSF4 Editing in Human Cells

Having designed and analyzed numerous sgRNA candidates that were predicted to edit the human OX40 Receptor protein to produce either a knockout or a dominant negative receptor (see FIG. 48H), the next step is to assess their ability to act in vitro. Of particular interest is whether the preferred (i.e., on-target) edit will be observed in edited cells.

To test this, 80 pmoles of sgRNA candidates (SEQ ID NOs: 3714-3740) are introduced into human THP-1 monocytes via electroporation as part of a ribonucleoprotein as previously described in Example 14.

It is anticipated that results of these studies will confirm the robustness of the in silico analysis and identify multiple candidates are indeed able to induce mutation in the TNFRSF4 gene at high efficacy. It is further expected that use of AR-Cas9 will improve editing efficacy (i.e., increase on-target editing) as compared to wtCas9.

### Example 36: In Vitro Delivery of TNFRSF4-Editing Guide RNA to Human Cells Via Lipid Nanoparticles

Because all validation experiments are to be conducted via electroporation of cell cultures to introduce RNA-dependent nucleases and sgRNAs, additional experiments will assess the suitability of using lipid nanoparticle (LNP) to deliver mRNA to human cells. LNPs containing either sgRNA (SEQ ID NOs: 3714-3740) or Cas9 mRNA are incubated with THP-1 monocytes, followed by validation of editing as described in Example 15. Upon confirmation of editing various functional assays as described in Example 13 will be performed.

### Example 37: In Vitro Delivery of TNFRSF4-Editing Guide RNA to Human Cells Via Adeno-Associated Virus

Because all validation experiments are to be conducted via electroporation of cell cultures to introduce RNA-dependent nucleases and sgRNAs, additional experiments will assess the suitability of using adeno-associated virus (AAV) vectors to deliver sgRNA to human cells. Exemplary AAV vectors are shown in Table 2. After being prepared according to manufacturer specifications, such that one or more of the sgRNAs containing SEQ ID NOs: 3714-3740 are integrated into the viral vector, said vectors are introduced to human U20S cells and editing is validated as previously described in Example 16. Upon confirmation of editing, various functional assays as described in Example 13 will be performed.

### Example 38: Validation of TNFRSF11A Editing in Human Cells

Having designed and analyzed numerous sgRNA candidates that were predicted to edit the human Tumor Necrosis Factor Receptor Superfamily Member 11A protein to produce either a knockout or a dominant negative receptor (see FIG. 48I), the next step is to assess their ability to act in vitro. Of particular interest is whether the preferred (i.e., on-target) edit will be observed in edited cells.

To test this, 80 pmoles of sgRNA candidates (SEQ ID NOs: 3741-3788) are introduced into human THP-1 monocytes via electroporation as part of a ribonucleoprotein as previously described in Example 14.

It is anticipated that results of these studies will confirm the robustness of the in silico analysis and identify multiple candidates are indeed able to induce mutation in the TNFRSF11A gene at high efficacy. It is further expected that use of AR-Cas9 will improve editing efficacy (i.e., increase on-target editing) as compared to wtCas9.

### Example 39: In Vitro Delivery of TNFRSF11A-Editing Guide RNA to Human Cells Via Lipid Nanoparticles

Because all validation experiments are to be conducted via electroporation of cell cultures to introduce RNA-dependent nucleases and sgRNAs, additional experiments will assess the suitability of using lipid nanoparticle (LNP) to deliver mRNA to human cells. LNPs containing either sgRNA (SEQ ID NOs: 3741-3788) or Cas9 mRNA are incubated with THP-1 monocytes, followed by validation of editing as described in Example 15. Upon confirmation of editing various functional assays as described in Example 13 will be performed.

### Example 40: In Vitro Delivery of TNFRSF11A-Editing Guide RNA to Human Cells Via Adeno-Associated Virus

Because all validation experiments are to be conducted via electroporation of cell cultures to introduce RNA-dependent nucleases and sgRNAs, additional experiments will assess the suitability of using adeno-associated virus (AAV) vectors to deliver sgRNA to human cells. Exemplary AAV vectors are shown in Table 2. After being prepared according to manufacturer specifications, such that one or more of the sgRNAs containing SEQ ID NOs: 3741-3788 are integrated into the viral vector, said vectors are introduced to human U2OS cells and editing is validated as previously described in Example 16. Upon confirmation of editing, various functional assays as described in Example 13 will be performed.

### Example 41: Validation of TGFBR1 Editing in Human Cells

Having designed and analyzed numerous sgRNA candidates that were predicted to edit the human Transforming Growth Factor Beta Receptor 1 protein to produce either a knockout or a dominant negative receptor (see FIG. 48J), the next step is to assess their ability to act in vitro. Of particular interest is whether the preferred (i.e., on-target) edit will be observed in edited cells.

To test this, 80 pmoles of sgRNA candidates (SEQ ID NOs: 3789-3813) are introduced into human THP-1 monocytes via electroporation as part of a ribonucleoprotein as previously described in Example 14.

It is anticipated that results of these studies will confirm the robustness of the in silico analysis and identify multiple candidates are indeed able to induce mutation in the TGFBR1 gene at high efficacy. It is further expected that use of AR-Cas9 will improve editing efficacy (i.e., increase on-target editing) as compared to wtCas9.

### Example 42: In Vitro Delivery of TGFBR1-Editing Guide RNA to Human Cells Via Lipid Nanoparticles

Because all validation experiments are to be conducted via electroporation of cell cultures to introduce RNA-dependent nucleases and sgRNAs, additional experiments will assess the suitability of using lipid nanoparticle (LNP) to deliver mRNA to human cells. LNPs containing either sgRNA (SEQ ID NOs: 3789-3813) or Cas9 mRNA are incubated with THP-1 monocytes, followed by validation of editing as described in Example 15. Upon confirmation of editing various functional assays as described in Example 13 will be performed.

### Example 43: In Vitro Delivery of TGFBR1-Editing Guide RNA to Human Cells Via Adeno-Associated Virus

Because all validation experiments are to be conducted via electroporation of cell cultures to introduce RNA-dependent nucleases and sgRNAs, additional experiments will assess the suitability of using adeno-associated virus (AAV) vectors to deliver sgRNA to human cells. Exemplary AAV vectors are shown in Table 2. After being prepared according to manufacturer specifications, such that one or more of the sgRNAs containing SEQ ID NOs: 3789-3813 are integrated into the viral vector, said vectors are introduced to human U2OS cells and editing is validated as previously described in Example 16. Upon confirmation of editing, various functional assays as described in Example 13 will be performed.

### Example 44: Validation of TGFBR2 Editing in Human Cells

Having designed and analyzed numerous sgRNA candidates that were predicted to edit the human Transforming Growth Factor Beta Receptor 2 protein to produce either a knockout or a dominant negative receptor (see FIG. 48K), the next step is to assess their ability to act in vitro. Of particular interest is whether the preferred (i.e., on-target) edit will be observed in edited cells.

To test this, 80 pmoles of sgRNA candidates (SEQ ID NOs: 3814-3865) are introduced into human THP-1 monocytes via electroporation as part of a ribonucleoprotein as previously described in Example 14.

It is anticipated that results of these studies will confirm the robustness of the in silico analysis and identify multiple candidates are indeed able to induce mutation in the TGFBR2 gene at high efficacy. It is further expected that use of AR-Cas9 will improve editing efficacy (i.e., increase on-target editing) as compared to wtCas9.

### Example 45: In Vitro Delivery of TGFBR2-Editing Guide RNA to Human Cells Via Lipid Nanoparticles

Because all validation experiments are to be conducted via electroporation of cell cultures to introduce RNA-dependent nucleases and sgRNAs, additional experiments will assess the suitability of using lipid nanoparticle (LNP) to deliver mRNA to human cells. LNPs containing either sgRNA (SEQ ID NOs: 3814-3865) or Cas9 mRNA are incubated with THP-1 monocytes, followed by validation of editing as described in Example 15. Upon confirmation of editing various functional assays as described in Example 13 will be performed.

### Example 46: In Vitro Delivery of TGFBR2-Editing Guide RNA to Human Cells Via Adeno-Associated Virus

Because all validation experiments are to be conducted via electroporation of cell cultures to introduce RNA-dependent nucleases and sgRNAs, additional experiments will assess the suitability of using adeno-associated virus (AAV) vectors to deliver sgRNA to human cells. Exemplary AAV vectors are shown in Table 2. After being prepared according to manufacturer specifications, such that one or more of the sgRNAs containing SEQ ID NOs: 3814-3865 are integrated into the viral vector, said vectors are introduced to human U2OS cells and editing is validated as previously described in Example 16. Upon confirmation of editing, various functional assays as described in Example 13 will be performed.

The examples set forth above are provided to give those of ordinary skill in the art a complete disclosure and description of how to make and use the embodiments of the compositions, systems and methods of the disclosure, and are not intended to limit the scope of what the inventors regard as their invention. Modifications of the above-described modes for carrying out the embodiments of the disclosure that are obvious to persons of skill in the art are intended to be within the scope of the following claims. All patents and publications mentioned in the specification are indicative of the levels of skill of those skilled in the art to which the disclosure pertains.

All headings and section designations are used for clarity and reference purposes only and are not to be considered limiting in any way. For example, those of skill in the art will appreciate the usefulness of combining various aspects from different headings and sections as appropriate according to the spirit and scope of the disclosure described herein.

It is to be understood that the methods described herein are not limited to the particular methodology, protocols, subjects, and sequencing techniques described herein and as such can vary. It is also to be understood that the terminology used herein is for the purpose of describing particular embodiments only, and is not intended to limit the scope of the methods and compositions described herein, which will be limited only by the appended claims. While some embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein can be employed in practicing the disclosure. It is intended that the following claims define the scope of the disclosure and that methods and structures within the scope of these claims and their equivalents be covered thereby.

The examples set forth above are provided to give those of ordinary skill in the art a complete disclosure and description of how to make and use the embodiments of the compositions, systems and methods of the disclosure, and are not intended to limit the scope of what the inventors regard as their invention. Modifications of the above-described modes for carrying out the embodiments of the disclosure that are obvious to persons of skill in the art are intended to be within the scope of the following claims. All patents and publications mentioned in the specification are indicative of the levels of skill of those skilled in the art to which the disclosure pertains.

All headings and section designations are used for clarity and reference purposes only and are not to be considered limiting in any way. For example, those of skill in the art will appreciate the usefulness of combining various aspects from different headings and sections as appropriate according to the spirit and scope of the disclosure described herein.

It is to be understood that the methods described herein are not limited to the particular methodology, protocols, subjects, and sequencing techniques described herein and as such can vary. It is also to be understood that the terminology used herein is for the purpose of describing particular embodiments only, and is not intended to limit the scope of the methods and compositions described herein, which will be limited only by the appended claims. While some embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein can be employed in practicing the disclosure. It is intended that the following claims define the scope of the disclosure and that methods and structures within the scope of these claims and their equivalents be covered thereby.

Several aspects are described with reference to example applications for illustration. Unless otherwise indicated, any embodiment can be combined with any other embodiment. It should be understood that numerous specific details, relationships, and methods are set forth to provide a full understanding of the features described herein. A skilled artisan, however, will readily recognize that the features described herein can be practiced without one or more of the specific details or with other methods. The features described herein are not limited by the illustrated ordering of acts or events, as some acts can occur in different orders and/or concurrently with other acts or events. Furthermore, not all illustrated acts or events are required to implement a methodology in accordance with the features described herein.

While some embodiments have been shown and described herein, it will be obvious to those skilled in the art that such embodiments are provided by way of example only. It is not intended that the disclosure be limited by the specific examples provided within the specification. While the disclosure has been described with reference to the aforementioned specification, the descriptions and illustrations of the embodiments herein are not meant to be construed in a limiting sense. Numerous variations, changes, and substitutions will now occur to those skilled in the art without departing from the disclosure.

Furthermore, it shall be understood that all aspects of the disclosure are not limited to the specific depictions, configurations or relative proportions set forth herein which depend upon a variety of conditions and variables. It should be understood that various alternatives to the embodiments of the disclosure described herein can be employed in practicing the disclosure. It is therefore contemplated that the disclosure shall also cover any such alternatives, modifications, variations or equivalents. It is intended that the following claims define the scope of the disclosure and that methods and structures within the scope of these claims and their equivalents be covered thereby.

All publications, patents, and patent applications herein are incorporated by reference to the same extent as if each individual publication, patent, or patent application was specifically and individually indicated to be incorporated by reference. In the event of a conflict between a term herein and a term in an incorporated reference, the term herein controls.

What is claimed is:

1. A pharmaceutical composition for treating a disorder having a symptom caused, at least in part, by intercellular signaling mediated through a transmembrane receptor, the composition comprising:
(i) an RNA-guided nuclease or a nucleic acid encoding an RNA-guided nuclease; and
(ii) at least one guide RNA or a nucleic acid encoding at least one guide RNA targeting a gene encoding the transmembrane receptor.

2. The pharmaceutical composition of claim 1, wherein the disorder is a musculoskeletal disorder.

3. The pharmaceutical composition of claim 2, wherein the musculoskeletal disorder is selected from the group consisting of Rheumatoid Arthritis, Gout, Osteoarthritis, Osteoporosis, Intervertebral disc disease (IVDD), Psoriatic arthritis (PsA), Arthritis, Polymyositis, Proliferative synovitis, Malignant bone neoplasm, Sarcoid Myopathy, Cortex Bone Disorders, Idiopathic Scoliosis, Tendinopathy, Myofibrillar Myopathy, Enthesis-Related Arthritis, Ankylosing spondylitis, Degenerative, polyarthritis, Arthropathy, Osteitis Deformans, Prolapsed Lumbar Disc, Polymyositis Ossificans, Idiopathic Polymyositis, Luft Disease, Adult-onset Still's Disease, Osteoarthrosis Deformans, and Bachet's Disease.

4. The pharmaceutical composition of claim 2, wherein the musculoskeletal disorder is selected from the group consisting of Rheumatoid arthritis, Idiopathic osteoporosis, Post-menopausal osteoporosis, Paget's disease, Osteoarthritis, Juvenile idiopathic arthritis (JIA), Still's disease, Ankylosing spondylitis, Polymyalgia rheumatica, Arthritis, Secondary malignant neoplasm of bone, Type II, Mucolipidosis, Sjogren's Syndrome, Psoriatic arthritis, Rheumatism, Castleman's disease, Degenerative Polyarthritis, Arthropathy, Bone neoplasm, Osteoporosis, Massive osteolysis, Bone fracture healing, Systemic sclerosis, Systemic Juvenile Idiopathic, Arthritis, and Synovitis.

5. The pharmaceutical composition of claim 2, wherein the musculoskeletal disorder is selected from the group consisting of Arthritis, Infectious Intermittent joint effusion, Ankylosing spondylitis, Arthritis, Osteoarthritis, Spondylarthrites, Plantar fasciitis, Degenerative polyarthritis, Hemophilic arthropathy, Inflammatory myopathy with abundant macrophages, Polymyositis, Tendinosis, Malignant Bone Neoplasm, Osteoporosis, Psoriatic arthritis, Rheumatism, Rheumatoid arthritis, Adult Still's disease, Juvenile arthritis, Early rheumatoid arthritis, Palindromic rheumatism, Gout, Infectious Arthritis, Myotonic dystrophy, Dermatomyositis, Hemophilic arthropathy, Osteopenia, Sjogren's syndrome, Juvenile Idiopathic Arthritis, Myasthenia gravis, Osteolysis, Inflammation, Degenerative polyarthritis, Osteitis deformans, Pigmented villonodular synovitis, or Hyperphosphatasemia with bone disease.

6. The pharmaceutical composition of claim 2, wherein the musculoskeletal disorder is selected from the group consisting of Loeys-Dietz Syndrome, Osteoarthrosis, Marfan Syndrome, Aortic aneurysm (familial thoracic 3), and a Craniofacial abnormality.

7. The pharmaceutical composition of claim 1, wherein the disorder is a neoplasia.

8. The pharmaceutical composition of claim 7, wherein the neoplasia is selected from the group consisting of Osteosarcoma, Colon Cancer, Metastasis (General), Lung Cancer, Multiple Myeloma. Breast Cancer, Solid Tumors, Lymphoma, Pancreatic Cancer, Stomach Cancer, Epithelial Ovarian Cancer, Mammary Neoplasms, Oropharyngeal Carcinoma, Renal Cell Carcinoma, Chondrosarcoma, Esophageal Neoplasms, B-Cell Lymphoma, Cutaneous Lymphoma T-Cell, Leukemia, Thyroid Carcinoma, Skin carcinogenesis, Cholectoral Cancer, Glioma, Liver Cancer, Melanoma, Neuroblastoma, Polycystic Ovary Syndrome, Glioblastoma, Prostate Cancer, Cervical Cancer, Ovarian Cancer, Bladder Cancer, Squamous cell carcinoma, Kaposi Sarcoma, Oral Cavity Cancer, Leiomyosarcoma, Malignant Peripheral Nerve Sheath Tumor, and Ewing's Sarcoma.

9. The pharmaceutical composition of claim 7, wherein the neoplasia is selected from the group consisting of Multiple myeloma, Lung Cancer, Stomach Cancer, Breast Cancer, Kidney Cancer, Neoplasm Metastasis, Colorectal Neoplasms, Ovarian cancer, Osteosarcoma, Cholangiocarcinoma, Leukemia, Prostate cancer, Plasmacytoma, Pancreatic cancer, Cervical cancer, Lymphoma, Liver neoplasms, Neuroblastoma, Melanoma, Mastocytosis, Endometrial cancer, Bladder Cancer, Squamous cell carcinoma, Gallbladder carcinoma, Adenocarcinoma, Thyroid Cancer, prostate neoplasms, stomach cancer, liver carcinoma, pituitary neoplasms, hepatoblastoma, multiple myeloma, hepatocellular adenoma, breast carcinoma, carcinogenesis, leukemia, colon carcinoma, melanoma, metastasis (general), lung cancer, pancreatic cancer, Kaposi sarcoma, medulloblastoma, carcinoma of bladder, squamous cell carcinoma, mast cell neoplasm, glioma, mastocytoma, brain neoplasms, ovarian neoplasms, bone neoplasm, rhabdomyosarcoma, solid tumors, metastatic kidney cancer, and metastatic renal cell carcinoma

10. The pharmaceutical composition of claim 7, wherein the neoplasia is selected from the group consisting of a Neoplasm, Glioblastoma, Astrocytoma, Adenocarcinoma, Osteosarcoma, Squamous cell carcinoma, Metastasis, Ameloblastoma, Cholangiocarcinoma, Choriocarcinoma, Ovarian cancer, Prostate cancer, Lung Cancer, Larynx Cancer, Breast Cancer, Lip and Oral Cavity Carcinoma, Squamous intraepithelial lesion, Neuroblastoma, Non-Hodgkin lymphomas, Malignant Neoplasms, Skin Neoplasms, Neuroblastoma, Neoplasm Metastasis, Colorectal Cancer, Fibrosarcoma, Myeloid Leukemia, Myelofibrosis, Hodgkin Lymphomas, Non-Small Cell Lung Carcinoma, Cervical Cancer, Liver cancer, Extrapulmonary small cell carcinoma, Basal cell carcinoma, Kidney cancer, Pancreatic carcinoma, Mesothelioma, Gastric cancer, Polycystic Ovary Syndrome, Chordoma, Cholangiocarcinoma, Malignant ascites, Nasopharyngeal carcinoma, Head and Neck Carcinoma, Cancer metastasis, Prostate carcinoma, Fibrosarcoma, Liver carcinoma, Carcinoma of bladder, T-Cell Lymphoblastic Leukemia, Bladder Neoplasm, melanoma, Leukemia, acute myeloid leukemia, Hepatocellular carcinoma, Colorectal cancer, a Lymphoma, Mycosis fungoides, and Sézary syndrome.

11. The pharmaceutical composition of claim 7, wherein the neoplasia is selected from the group consisting of Pancreatic cancer, Multiple self-healing squamous epithelioma (Ferguson-Smith disease), Pancreatic cancer, Gastrointestinal Stromal Tumors (GIST), Hereditary Nonpolyposis Colorectal Cancer (Lynch Syndrome), Metastasis, Colorectal Carcinoma, Bone neoplasms, Anaplastic carcinoma, Spindle-Cell carcinoma, Malignant Bone Neoplasm, Lung neoplasms, and Malignant brain neoplasm.

12. The pharmaceutical composition of claim 1, wherein the disorder is a neurological disorder.

13. The pharmaceutical composition of claim 12, wherein the neurological disorder is selected from the group consisting of Acute Thrombotic Stroke, Epilepsy, Multiple Sclerosis, and Alzheimer's Disease.

14. The pharmaceutical composition of claim 1, wherein the disorder is a cardiac disorder.

15. The pharmaceutical composition of claim 14, wherein the cardiac disorder is selected from the group consisting of Acute Myocardial Infarction, refractory heart failure, arrhythmias, pericarditis, myocarditis, sepsis-induced cardiomyopathy, Acute Decompensated Heart Failure, Refractory Idiopathic Pericarditis, Atherosclerosis, coronary artery disease, myocardial infarction, and cardiac remodeling.

16. The pharmaceutical composition of claim 14, wherein the cardiac disorder is atherosclerosis or abdominal aortic aneurism.

17. The pharmaceutical composition of claim 1, wherein the disorder is an inflammatory disorder.

18. The pharmaceutical composition of claim 17, wherein the inflammatory disorder is selected from the group consisting of Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), and Fibrosis.

19. The pharmaceutical composition of claim 17, wherein the inflammatory disorder is selected from the group consisting of a cytokine storm, acute local inflammation, inflammatory bowel disease, Crohn's disease, sepsis, Experimental sepsis, and Castleman's disease

20. The pharmaceutical composition of claim 1, wherein the disorder is a digestive disorder.

21. The pharmaceutical composition of claim 20, wherein the digestive disorder is selected from the group consisting of Inflammatory Bowel Disease (IBD), Gastroesophageal reflux disease (GERD), and Non-HP-associated Peptic Ulcer Disease.

22. The pharmaceutical composition of claim 1, wherein the disorder is a respiratory disorder.

23. The pharmaceutical composition of claim 22, wherein the respiratory disorder is Asthma or Pulmonary Fibrosis.

24. The pharmaceutical composition of claim 1, wherein the disorder is a renal, metabolic, or ophthalmic disorder.

25. The pharmaceutical composition of claim 24, wherein the renal, metabolic, or ophthalmic disorder is selected from the group consisting of Chronic Kidney Disease, Type 2 Diabetes, an Eye Disease, Uveitis, Scleritis, Sjogren asthenia, and dry eye.

26. The pharmaceutical composition of claim 1, wherein the disorder is an autoimmune disorder.

27. The pharmaceutical composition of claim 26, wherein the autoimmune disorder is Systemic Lupus Erythematosus.

28. The pharmaceutical composition of claim 1, wherein the disorder is acute synovitis, chronic synovitis, inflammatory arthritis, or immune-mediated arthritides.

29. The pharmaceutical composition of claim 1, wherein the disorder is an autoimmune or inflammatory disorder.

30. The pharmaceutical composition of claim 28, wherein the autoimmune or inflammatory disorder is selected from the group consisting of an Allergy, Asthma, Multiple sclerosis, Psoriasis, Inflammatory bowel disease, Autoimmune experimental uveitis, Colitis, Hypersensitivity reaction disease, Diabetes, Crohn's disease, Systemic lupus erythematosus, Pulmonary sarcoidosis, Leishmaniasis, Experimental autoimmune encephalomyelitis, HTLV-1-associated myelopathy, Tropical spastic paraparesis, Scleroderma, an Idiopathic inflammatory myopathy, polymyositis, and dermatomyositis.

31. The pharmaceutical composition of any one of claims 1-30, wherein the transmembrane receptor is an interleukin-1 receptor.

32. The pharmaceutical composition of claim 31, wherein the at least one guide RNA targets a gene selected from the group consisting of IL1R1, IL1RAP, IL1R5, IL1R6, IL1R7, and IL1R9.

33. The pharmaceutical composition of claim 31, wherein the at least one guide RNA targets an IL1R1 gene.

34. The pharmaceutical composition of claim 33, wherein the at least one guide RNA comprises a crRNA sequence selected from the group consisting of SEQ ID NOS: 680-811.

35. The pharmaceutical composition of claim 33, wherein the at least one guide RNA comprises a crRNA sequence selected from the group consisting of SEQ ID NOS: 825-892 and 3336-3405.

36. The pharmaceutical composition of claim 33, wherein the at least one guide RNA comprises a crRNA sequence selected from the group consisting of SEQ ID NOS: 893-967.

37. The pharmaceutical composition of claim 33, wherein the at least one guide RNA comprises a crRNA sequence selected from the group consisting of SEQ ID NOS: 968-1039.

38. The pharmaceutical composition of claim 33, wherein the at least one guide RNA comprises a crRNA sequence selected from the group consisting of SEQ ID NOS: 3491-3513.

39. The pharmaceutical composition of claim 31, wherein the at least one guide RNA targets an IL1RAP gene.

40. The pharmaceutical composition of claim 39, wherein the at least one guide RNA comprises a crRNA sequence selected from the group consisting of SEQ ID NOS: 1040-1194.

41. The pharmaceutical composition of claim 39, wherein the at least one guide RNA comprises a crRNA sequence selected from the group consisting of SEQ ID NOS: 1204-1271 and 3421-3490.

42. The pharmaceutical composition of claim 39, wherein the at least one guide RNA comprises a crRNA sequence selected from the group consisting of SEQ ID NOS: 1272-1348.

43. The pharmaceutical composition of claim 39, wherein the at least one guide RNA comprises a crRNA sequence selected from the group consisting of SEQ ID NOS: 1349-1424.

44. The pharmaceutical composition of claim 39, wherein the at least one guide RNA comprises a crRNA sequence selected from the group consisting of SEQ ID NOS: 3514-3543.

45. The pharmaceutical composition of any one of claims 1-30, wherein the transmembrane receptor is an interleukin-6 receptor.

46. The pharmaceutical composition of claim 45, wherein the at least one guide RNA targets an IL6R gene.

47. The pharmaceutical composition of claim 46, wherein the at least one guide RNA comprises a crRNA sequence selected from the group consisting of SEQ ID NOS: 1746-1920.

48. The pharmaceutical composition of claim 46, wherein the at least one guide RNA comprises a crRNA sequence selected from the group consisting of SEQ ID NOS: 3544-3566.

49. The pharmaceutical composition of claim 45, wherein the at least one guide RNA targets an IL6ST gene.

50. The pharmaceutical composition of claim 49, wherein the at least one guide RNA comprises a crRNA sequence selected from the group consisting of SEQ ID NOS: 1969-2176.

51. The pharmaceutical composition of claim 49, wherein the at least one guide RNA comprises a crRNA sequence selected from the group consisting of SEQ ID NOS: 3567-3606.

52. The pharmaceutical composition of any one of claims 1-30, wherein the transmembrane receptor is a tumor necrosis factor receptor.

53. The pharmaceutical composition of claim 52, wherein the at least one guide RNA targets a TNFRSF1A gene.

54. The pharmaceutical composition of claim 53, wherein the at least one guide RNA comprises a crRNA sequence selected from the group consisting of SEQ ID NOS: 2179-2383.

55. The pharmaceutical composition of claim 53, wherein the at least one guide RNA comprises a crRNA sequence selected from the group consisting of SEQ ID NOS: 3607-3647.

56. The pharmaceutical composition of claim 52, wherein the at least one guide RNA targets a TNFRSF1B gene.

57. The pharmaceutical composition of claim 56, wherein the at least one guide RNA comprises a crRNA sequence selected from the group consisting of SEQ ID NOS: 2396-2622.

58. The pharmaceutical composition of claim 56, wherein the at least one guide RNA comprises a crRNA sequence selected from the group consisting of SEQ ID NOS: 3648-3692.

59. The pharmaceutical composition of claim 52, wherein the at least one guide RNA targets a TNFRSF3 gene.

60. The pharmaceutical composition of claim 59, wherein the at least one guide RNA comprises a crRNA sequence selected from the group consisting of SEQ ID NOS: 2643-2852.

61. The pharmaceutical composition of claim 59, wherein the at least one guide RNA comprises a crRNA sequence selected from the group consisting of SEQ ID NOS: 3693-3713.

62. The pharmaceutical composition of claim 52, wherein the at least one guide RNA targets a TNFRSF4 gene.

63. The pharmaceutical composition of claim 62, wherein the at least one guide RNA comprises a crRNA sequence selected from the group consisting of SEQ ID NOS: 2867-3035.

64. The pharmaceutical composition of claim 62, wherein the at least one guide RNA comprises a crRNA sequence selected from the group consisting of SEQ ID NOS: 3714-3740.

65. The pharmaceutical composition of claim 52, wherein the at least one guide RNA targets a TNFRSF11A gene.

66. The pharmaceutical composition of claim 65, wherein the at least one guide RNA comprises a crRNA sequence selected from the group consisting of SEQ ID NOS: 3042-3331.

67. The pharmaceutical composition of claim 65, wherein the at least one guide RNA comprises a crRNA sequence selected from the group consisting of SEQ ID NOS: 3741-3788.

68. The pharmaceutical composition of any one of claims 1-30, wherein the transmembrane receptor is a transforming growth factor beta receptor.

69. The pharmaceutical composition of claim 68, wherein the at least one guide RNA targets a TGFBR1 gene.

70. The pharmaceutical composition of claim 69, wherein the at least one guide RNA comprises a crRNA sequence selected from the group consisting of SEQ ID NOS: 1425-1544.

71. The pharmaceutical composition of claim 69, wherein the at least one guide RNA comprises a crRNA sequence selected from the group consisting of SEQ ID NOS: 3789-3813.

72. The pharmaceutical composition of claim 68, wherein the at least one guide RNA targets a TGFBR2 gene.

73. The pharmaceutical composition of claim 72, wherein the at least one guide RNA comprises a crRNA sequence selected from the group consisting of SEQ ID NOS: 1547-1734.

74. The pharmaceutical composition of claim 72, wherein the at least one guide RNA comprises a crRNA sequence selected from the group consisting of SEQ ID NOS: 3814-3865.

75. The pharmaceutical composition of any one of claims 1-73, wherein the RNA-guided nuclease or a nucleic acid encoding an RNA-guided nuclease is the RNA-guided nuclease.

76. The pharmaceutical composition of any one of claims 1-73, wherein the RNA-guided nuclease or a nucleic acid encoding an RNA-guided nuclease is DNA encoding the RNA-guided nuclease.

77. The pharmaceutical composition of any one of claims 1-73, wherein the RNA-guided nuclease or a nucleic acid encoding an RNA-guided nuclease is mRNA encoding the RNA-guided nuclease.

78. The pharmaceutical composition of any one of claims 1-77, wherein the RNA-guided nuclease is a Cas protein.

79. The pharmaceutical composition of claim 78, wherein the Cas protein is a Cas9 protein.

80. The pharmaceutical composition of claim 78, wherein the Cas9 protein is an S. pyogenes Cas9 polypeptide.

81. The pharmaceutical composition of claim 78, wherein the Cas9 protein is selected from the group consisting of esCas9, hfCas9, peCas9, and ARCas9.

82. The pharmaceutical composition of any one of claims 1-81, wherein the at least one guide RNA or a nucleic acid encoding at least one guide RNA is the at least one guide RNA.

83. The pharmaceutical composition of any one of claims 1-81, wherein the at least one guide RNA or a nucleic acid encoding at least one guide RNA is DNA encoding the at least one guide RNA.

84. The pharmaceutical composition of any one of claims 1-81, comprising a nucleic acid encoding both the RNA-guided nuclease and the at least one guide RNA.

85. The pharmaceutical composition of any one of claims 1-84, wherein the at least one guide RNA is a single guide RNA (sgRNA).

86. The pharmaceutical composition of any one of claims 1-85, wherein the at least one guide RNA targets a human gene.

87. The pharmaceutical composition of any one of claims 1-85, wherein the at least one guide RNA targets a canine gene.

88. The pharmaceutical composition of any one of claims 1-85, wherein the at least one guide RNA targets an equine gene.

89. The pharmaceutical composition of any one of claims 1-85, wherein the at least one guide RNA targets a feline gene.

90. The pharmaceutical composition of any one of claims 1-85, wherein the at least one guide RNA targets a mammalian gene.

91. The pharmaceutical composition of any one of claims 1-90, wherein the composition comprises one or more viral vectors collectively comprising the (i) RNA-guided nuclease or a nucleic acid encoding an RNA-guided nuclease, and (ii) at least one guide RNA or a nucleic acid encoding at least one guide RNA targeting a gene encoding the transmembrane receptor.

92. The pharmaceutical composition of claim 91, wherein the one of more viral vectors comprise a recombinant virus selected from a retrovirus, an adenovirus, an adeno-associated virus, a lentivirus, and a herpes simplex virus-1.

93. The pharmaceutical composition of claim 91, wherein the one of more viral vectors comprise a recombinant adeno-associated virus (AAV).

94. The pharmaceutical composition of claim 93, wherein the recombinant AAV is of serotype 5 (AAV5).

95. The pharmaceutical composition of claim 93, wherein the recombinant AAV is of serotype 6 (AAV6).

96. The pharmaceutical composition of any one of claims 1-90, wherein the composition comprises one or more lipid nanoparticles (LNP) collectively comprising the (i) RNA-guided nuclease or a nucleic acid encoding an RNA-guided nuclease, and (ii) at least one guide RNA or a nucleic acid encoding at least one guide RNA targeting a gene encoding the transmembrane receptor.

97. The pharmaceutical composition of claim 96, wherein the one or more LNP comprises:
a first plurality of LNP encapsulating the RNA-guided nuclease or a nucleic acid encoding an RNA-guided nuclease; and
a second plurality of LNP encapsulating the at least one guide RNA or a nucleic acid encoding at least one guide RNA.

98. The pharmaceutical composition of claim 96, wherein the one or more LNP comprises a plurality of LNP encapsulating both the (i) RNA-guided nuclease or a nucleic acid encoding an RNA-guided nuclease, and (ii) at least one guide RNA or a nucleic acid encoding at least one guide RNA targeting a gene encoding the transmembrane receptor.

99. The pharmaceutical composition of any one of claims 96-98, wherein the one or more LNP comprises a component selected from the group consisting of 3-(didodecylamino)-N1,N1,4-tridodecyl-1-piperazineethanamine (KL10), N1-[2-(didodecylamino)ethyl]-N1,N4,N4-tridodecyl-1,4-piperazinediethanamine (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl 4-(dimethylamino) butanoate (DLin-MC3-DMA), 2,2-dilinoleyl-4-(2-dimethylaminoethyl)-[1,3]-dioxolane (DLin-KC2-DMA), 1,2-dioleyloxy-N,N-dimethylaminopropane (DODMA), 2-({8-[(3.beta.)-cholest-5-en-3-yloxy]octyl}oxy)-N,N-dimethyl-3-[(9Z,12Z)-octadeca-9,12-dien-1-yloxy]propan-1-amine (Octyl-CLinDMA), (2R)-2-({8-[(3.beta.)-cholest-5-en-3-yloxy]octyl}oxy)-N,N-dimethyl-3-[(9Z-,12Z)-octadeca-9,12-dien-1-yloxy]propan-1-amine (Octyl-CLinDMA (2R)), (2S)-2-({8-[(3.beta.)-cholest-5-en-3-yloxy]octyl}oxy)-N,N-dimethyl-3-[(9Z-,12Z)-octadeca-9,12-dien-1-yloxy]propan-1-amine (Octyl-CLinDMA (2S)), a lipid including a cyclic amine group, and a mixture thereof.

100. The pharmaceutical composition of any one of claims 96-99, wherein the LNP comprises a component selected from the group consisting of 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanolamine (DOPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin (SM), and a mixture thereof.

101. The pharmaceutical composition of any one of claims 96-296, wherein the LNP comprises a component selected from the group consisting of PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG-DLPE, PEG-DMPE, PEG-DPPC, PEG-DMA, a PEG-DSPE lipid, and a mixture thereof.

102. The pharmaceutical composition of any one of claims 96-100, wherein the LNP comprises a component selected from the group consisting of a cholesterol, fecosterol, stigmasterol, stigmastanol, sitosterol, β-sitosterol, lupeol, betulin, ursolic acid, oleanolic acid, campesterol, fucosterol, brassicasterol, ergosterol, 9,11-dehydroergosterol, tomatidine, tomatine, α-tocopherol, and a mixture thereof.

103. The pharmaceutical composition of any one of claims 1-90, wherein the composition comprises one or more liposomes collectively comprising the (i) RNA-guided nuclease or a nucleic acid encoding an RNA-guided nuclease, and (ii) at least one guide RNA or a nucleic acid encoding at least one guide RNA targeting a gene encoding the transmembrane receptor.

104. The pharmaceutical composition of claim 103, wherein the one or more liposomes comprises:
a first plurality of liposomes encapsulating the RNA-guided nuclease or a nucleic acid encoding an RNA-guided nuclease; and
a second plurality of liposomes encapsulating the at least one guide RNA or a nucleic acid encoding at least one guide RNA.

105. The pharmaceutical composition of claim 103, wherein the one or more liposomes comprises a plurality of liposomes encapsulating both the (i) RNA-guided nuclease or a nucleic acid encoding an RNA-guided nuclease, and (ii) at least one guide RNA or a nucleic acid encoding at least one guide RNA targeting a gene encoding the transmembrane receptor.

106. The pharmaceutical composition of any one of claims 1-90, wherein the composition comprises one or more virus-like particles collectively comprising the (i) RNA-guided nuclease or a nucleic acid encoding an RNA-guided nuclease, and (ii) at least one guide RNA or a nucleic acid encoding at least one guide RNA targeting a gene encoding the transmembrane receptor.

107. The pharmaceutical composition of claim 106, wherein the one or more virus-like particles comprises:
a first plurality of virus-like particles encapsulating the RNA-guided nuclease or a nucleic acid encoding an RNA-guided nuclease; and
a second plurality of virus-like particles encapsulating the at least one guide RNA or a nucleic acid encoding at least one guide RNA.

108. The pharmaceutical composition of claim 106, wherein the one or more virus-like particles comprises a plurality of virus-like particles encapsulating both the (i) RNA-guided nuclease or a nucleic acid encoding an RNA-guided nuclease, and (ii) at least one guide RNA or a nucleic acid encoding at least one guide RNA targeting a gene encoding the transmembrane receptor.

109. The pharmaceutical composition of any one of claims 1-108, wherein the composition is formulated for parenteral administration.

110. The pharmaceutical composition of any one of claims 1-108, wherein the composition is formulated for intra-articular injection within a joint of the subject.

111. The pharmaceutical composition of any one of claims 1-108, wherein the composition is formulated for intradiscal injection.

112. The pharmaceutical composition of any one of claims 1-108, wherein the composition is formulated for peridiscal injection.

113. The pharmaceutical composition of any one of claims 1-108, wherein the composition is formulated for intravertebral injection.

114. A method for treating a disorder having a symptom caused, at least in part, by intracellular signaling mediated through a transmembrane receptor in a subject in need thereof, comprising:
administering a therapeutically effective amount of a composition, wherein the composition comprises:
(i) an RNA-guided nuclease or a nucleic acid encoding an RNA-guided nuclease; and
(ii) at least one guide RNA or a nucleic acid encoding at least one guide RNA targeting a gene encoding the transmembrane receptor.

115. The method of claim 114, wherein the at least one guide RNA targets a gene selected from the group consisting of IL1R1, IL1RAP, IL1R5, IL1R6, IL1R7, and IL1R9.

116. The method of claim 114, wherein the at least one guide RNA targets an IL1R1 gene.

117. The method of claim 116, wherein the at least one guide RNA comprises a crRNA sequence selected from the group consisting of SEQ ID NOS: 680-758.

118. The method of claim 116, wherein the at least one guide RNA comprises a crRNA sequence selected from the group consisting of SEQ ID NOS: 825-864.

119. The method of claim 116, wherein the at least one guide RNA comprises a crRNA sequence selected from the group consisting of SEQ ID NOS: 893-922.

120. The method of claim 116, wherein the at least one guide RNA comprises a crRNA sequence selected from the group consisting of SEQ ID NOS: 968-997.

121. The method of claim 116, wherein the at least one guide RNA comprises a crRNA sequence selected from the group consisting of SEQ ID NOS: 3491-3503.

122. The method of claim 114, wherein the at least one guide RNA targets an IL1RAP gene.

123. The method of claim 122, wherein the at least one guide RNA comprises a crRNA sequence selected from the group consisting of SEQ ID NOS: 1040-1122.

124. The method of claim 122, wherein the at least one guide RNA comprises a crRNA sequence selected from the group consisting of SEQ ID NOS: 1204-1271 and 3421-3458.

125. The method of claim 122, wherein the at least one guide RNA comprises a crRNA sequence selected from the group consisting of SEQ ID NOS: 1272-1301.

126. The method of claim 122, wherein the at least one guide RNA comprises a crRNA sequence selected from the group consisting of SEQ ID NOS: 1349-1378.

127. The method of claim 122, wherein the at least one guide RNA comprises a crRNA sequence selected from the group consisting of SEQ ID NOS: 3514-3529.

128. The method of claim 114, wherein the transmembrane receptor is an interleukin-6 receptor.

129. The method of claim 128, wherein the at least one guide RNA targets an IL6R gene.

130. The method of claim 129, wherein the at least one guide RNA comprises a crRNA sequence selected from the group consisting of SEQ ID NOS: 1746-1834.

131. The method of claim 129, wherein the at least one guide RNA comprises a crRNA sequence selected from the group consisting of SEQ ID NOS: 3544-3553.

132. The method of claim 128, wherein the at least one guide RNA targets an IL6ST gene.

133. The method of claim 132, wherein the at least one guide RNA comprises a crRNA sequence selected from the group consisting of SEQ ID NOS: 1969-2037.

134. The method of claim 132, wherein the at least one guide RNA comprises a crRNA sequence selected from the group consisting of SEQ ID NOS: 3567-3574.

135. The method of claim 114, wherein the transmembrane receptor is a tumor necrosis factor receptor.

136. The method of claim 135, wherein the at least one guide RNA targets a TNFRSF1A gene.

137. The method of claim 136, wherein the at least one guide RNA comprises a crRNA sequence selected from the group consisting of SEQ ID NOS: 2179-2247.

138. The method of claim 136, wherein the at least one guide RNA comprises a crRNA sequence selected from the group consisting of SEQ ID NOS: 3607-3621.

139. The method of claim 135, wherein the at least one guide RNA targets a TNFRSF1B gene.

140. The method of claim 139, wherein the at least one guide RNA comprises a crRNA sequence selected from the group consisting of SEQ ID NOS: 2396-2482.

141. The method of claim 139, wherein the at least one guide RNA comprises a crRNA sequence selected from the group consisting of SEQ ID NOS: 3648-3673.

142. The method of claim 135, wherein the at least one guide RNA targets a TNFRSF3 gene.

143. The method of claim 142, wherein the at least one guide RNA comprises a crRNA sequence selected from the group consisting of SEQ ID NOS: 2643-2737.

144. The method of claim 142, wherein the at least one guide RNA comprises a crRNA sequence selected from the group consisting of SEQ ID NOS: 3693-3698.

145. The method of claim 135, wherein the at least one guide RNA targets a TNFRSF4 gene.

146. The method of claim 145, wherein the at least one guide RNA comprises a crRNA sequence selected from the group consisting of SEQ ID NOS: 2867-2950.

147. The method of claim 145, wherein the at least one guide RNA comprises a crRNA sequence selected from the group consisting of SEQ ID NOS: 3714-3734.

148. The method of claim 135, wherein the at least one guide RNA targets a TNFRSF11A gene.

149. The method of claim 148, wherein the at least one guide RNA comprises a crRNA sequence selected from the group consisting of SEQ ID NOS: 3042-3106.

150. The method of claim 148, wherein the at least one guide RNA comprises a crRNA sequence selected from the group consisting of SEQ ID NOS: 3741-3754.

151. The method of claim 114, wherein the transmembrane receptor is a transforming growth factor beta receptor.

152. The method of claim 151, wherein the at least one guide RNA targets a TGFBR1 gene.

153. The method of claim 152, wherein the at least one guide RNA comprises a crRNA sequence selected from the group consisting of SEQ ID NOS: 1425-1454.

154. The method of claim 152, wherein the at least one guide RNA comprises a crRNA sequence selected from the group consisting of SEQ ID NOS: 3789-3791.

155. The method of claim 151, wherein the at least one guide RNA targets a TGFBR2 gene.

156. The method of claim 155, wherein the at least one guide RNA comprises a crRNA sequence selected from the group consisting of SEQ ID NOS: 1547-1580.

157. The method of claim 155, wherein the at least one guide RNA comprises a crRNA sequence selected from the group consisting of SEQ ID NOS: 3814-3819.

158. The method of claim 114, wherein editing of a genomic copy of the gene for the transmembrane receptor by the RNA-guided nuclease and at least one guide RNA results in a genomic sequence encoding a membrane-bound form of the transmembrane receptor lacking signaling function.

159. The method of claim 158, wherein the at least one guide RNA targets a gene selected from the group consisting of IL1R1, IL1RAP, IL1R5, IL1R6, IL1R7, and IL1R9.

160. The method of claim 158, wherein the at least one guide RNA targets an IL1R1 gene.

161. The method of claim 160, wherein the at least one guide RNA comprises a crRNA sequence selected from the group consisting of SEQ ID NOS: 759-808.

162. The method of claim 160, wherein the at least one guide RNA comprises a crRNA sequence selected from the group consisting of SEQ ID NOS: 3336-3401.

163. The method of claim 160, wherein the at least one guide RNA comprises a crRNA sequence selected from the group consisting of SEQ ID NOS: 942-967.

164. The method of claim 160, wherein the at least one guide RNA comprises a crRNA sequence selected from the group consisting of SEQ ID NOS: 1010-1039.

165. The method of claim 160, wherein the at least one guide RNA comprises a crRNA sequence selected from the group consisting of SEQ ID NOS: 3505-3513.

166. The method of claim 158, wherein the at least one guide RNA targets an IL1RAP gene.

167. The method of claim 166, wherein the at least one guide RNA comprises a crRNA sequence selected from the group consisting of SEQ ID NOS: 1123-1180.

168. The method of claim 166, wherein the at least one guide RNA comprises a crRNA sequence selected from the group consisting of SEQ ID NOS: 3459-3485.

169. The method of claim 166, wherein the at least one guide RNA comprises a crRNA sequence selected from the group consisting of SEQ ID NOS: 1323-1348.

170. The method of claim 166, wherein the at least one guide RNA comprises a crRNA sequence selected from the group consisting of SEQ ID NOS: 1397-1424.

171. The method of claim 166, wherein the at least one guide RNA comprises a crRNA sequence selected from the group consisting of SEQ ID NOS: 3533-3543.

172. The method of claim 158, wherein the transmembrane receptor is an interleukin-6 receptor.

173. The method of claim 172, wherein the at least one guide RNA targets an IL6ST gene.

174. The method of claim 173, wherein the at least one guide RNA comprises a crRNA sequence selected from the group consisting of SEQ ID NOS: 2038-2102.

175. The method of claim 173, wherein the at least one guide RNA comprises a crRNA sequence selected from the group consisting of SEQ ID NOS: 3595-3606.

176. The method of claim 158, wherein the transmembrane receptor is a tumor necrosis factor receptor.

177. The method of claim 176, wherein the at least one guide RNA targets a TNFRSF1A gene.

178. The method of claim 177, wherein the at least one guide RNA comprises a crRNA sequence selected from the group consisting of SEQ ID NOS: 2248-2369.

179. The method of claim 177, wherein the at least one guide RNA comprises a crRNA sequence selected from the group consisting of SEQ ID NOS: 3623-3647.

180. The method of claim 176, wherein the at least one guide RNA targets a TNFRSF1B gene.

181. The method of claim 180, wherein the at least one guide RNA comprises a crRNA sequence selected from the group consisting of SEQ ID NOS: 2483-2574.

182. The method of claim 180, wherein the at least one guide RNA comprises a crRNA sequence selected from the group consisting of SEQ ID NOS: 3681-3692.

183. The method of claim 176, wherein the at least one guide RNA targets a TNFRSF3 gene.

184. The method of claim 183, wherein the at least one guide RNA comprises a crRNA sequence selected from the group consisting of SEQ ID NOS: 2738-2846.

185. The method of claim 183, wherein the at least one guide RNA comprises a crRNA sequence selected from the group consisting of SEQ ID NOS: 3703-3713.

186. The method of claim 176, wherein the at least one guide RNA targets a TNFRSF4 gene.

187. The method of claim 186, wherein the at least one guide RNA comprises a crRNA sequence selected from the group consisting of SEQ ID NOS: 2951-2981.

188. The method of claim 186, wherein the at least one guide RNA comprises a crRNA sequence selected from the group consisting of SEQ ID NOS: 3738-3740.

189. The method of claim 176, wherein the at least one guide RNA targets a TNFRSF11A gene.

190. The method of claim 189, wherein the at least one guide RNA comprises a crRNA sequence selected from the group consisting of SEQ ID NOS: 3107-3322.

191. The method of claim 189, wherein the at least one guide RNA comprises a crRNA sequence selected from the group consisting of SEQ ID NOS: 3757-3788.

192. The method of claim 158, wherein the transmembrane receptor is a transforming growth factor beta receptor.

193. The method of claim 192, wherein the at least one guide RNA targets a TGFBR1 gene.

194. The method of claim 193, wherein the at least one guide RNA comprises a crRNA sequence selected from the group consisting of SEQ ID NOS: 1455-1534.

195. The method of claim 193, wherein the at least one guide RNA comprises a crRNA sequence selected from the group consisting of SEQ ID NOS: 3792-3813.

196. The method of claim 192, wherein the at least one guide RNA targets a TGFBR2 gene.

197. The method of claim 196, wherein the at least one guide RNA comprises a crRNA sequence selected from the group consisting of SEQ ID NOS: 1581-1724.

198. The method of claim 196, wherein the at least one guide RNA comprises a crRNA sequence selected from the group consisting of SEQ ID NOS: 3821-3865.

199. The method of claim 114, wherein editing of a genomic copy of the gene for the transmembrane receptor by the RNA-guided nuclease and at least one guide RNA results in a genomic sequence encoding a soluble form of the transmembrane receptor.

200. The method of claim 199, wherein the at least one guide RNA targets a gene selected from the group consisting of IL1R1, IL1RAP, IL1R5, IL1R6, IL1R7, and IL1R9.

201. The method of claim 200, wherein the at least one guide RNA targets an IL1R1 gene.

202. The method of claim 201, wherein the at least one guide RNA comprises a crRNA sequence selected from the group consisting of SEQ ID NOS: 809-811.

203. The method of claim 201, wherein the at least one guide RNA comprises a crRNA sequence selected from the group consisting of SEQ ID NOS: 3402-3405.

204. The method of claim 201, wherein the at least one guide RNA comprises a crRNA sequence selected from the group consisting of SEQ ID NOS: 923-941.

205. The method of claim 201, wherein the at least one guide RNA comprises a crRNA sequence selected from the group consisting of SEQ ID NOS: 998-1009.

206. The method of claim 201, wherein the at least one guide RNA comprises a crRNA sequence of SEQ ID NO 3504.

207. The method of claim 200, wherein the at least one guide RNA targets an IL1RAP gene.

208. The method of claim 207, wherein the at least one guide RNA comprises a crRNA sequence selected from the group consisting of SEQ ID NOS: 1181-1194.

209. The method of claim 207, wherein the at least one guide RNA comprises a crRNA sequence selected from the group consisting of SEQ ID NOS: 3481-3490.

210. The method of claim 207, wherein the at least one guide RNA comprises a crRNA sequence selected from the group consisting of SEQ ID NOS: 1302-1322.

211. The method of claim 207, wherein the at least one guide RNA comprises a crRNA sequence selected from the group consisting of SEQ ID NOS: 1379-1396.

212. The method of claim 207, wherein the at least one guide RNA comprises a crRNA sequence selected from the group consisting of SEQ ID NOS: 3530-3532.

213. The method of claim 199, wherein the transmembrane receptor is an interleukin-6 receptor.

214. The method of claim 213, wherein the at least one guide RNA targets an IL6R gene.

215. The method of claim 214, wherein the at least one guide RNA comprises a crRNA sequence selected from the group consisting of SEQ ID NOS: 1835-1920.

216. The method of claim 214, wherein the at least one guide RNA comprises a crRNA sequence selected from the group consisting of SEQ ID NOS: 3554-3566.

217. The method of claim 213, wherein the at least one guide RNA targets an IL6ST gene.

218. The method of claim 217, wherein the at least one guide RNA comprises a crRNA sequence selected from the group consisting of SEQ ID NOS: 2103-2176.

219. The method of claim 217, wherein the at least one guide RNA comprises a crRNA sequence selected from the group consisting of SEQ ID NOS: 3575-3594.

220. The method of claim 199, wherein the transmembrane receptor is a tumor necrosis factor receptor.

221. The method of claim 220, wherein the at least one guide RNA targets a TNFRSF1A gene.

222. The method of claim 221, wherein the at least one guide RNA comprises a crRNA sequence selected from the group consisting of SEQ ID NOS: 2370-2383.

223. The method of claim 221, wherein the at least one guide RNA comprises a crRNA sequence selected from the group consisting of SEQ ID NOS: 3621-3622.

224. The method of claim 220, wherein the at least one guide RNA targets a TNFRSF1B gene.

225. The method of claim 224, wherein the at least one guide RNA comprises a crRNA sequence selected from the group consisting of SEQ ID NOS: 2575-2622.

226. The method of claim 224, wherein the at least one guide RNA comprises a crRNA sequence selected from the group consisting of SEQ ID NOS: 3664-3680.

227. The method of claim 220, wherein the at least one guide RNA targets a TNFRSF3 gene.

228. The method of claim 227, wherein the at least one guide RNA comprises a crRNA sequence selected from the group consisting of SEQ ID NOS: 2847-2852.

229. The method of claim 227, wherein the at least one guide RNA comprises a crRNA sequence selected from the group consisting of SEQ ID NOS: 3699-3702.

230. The method of claim 220, wherein the at least one guide RNA targets a TNFRSF4 gene.

231. The method of claim 230, wherein the at least one guide RNA comprises a crRNA sequence selected from the group consisting of SEQ ID NOS: 2982-3035.

232. The method of claim 230, wherein the at least one guide RNA comprises a crRNA sequence selected from the group consisting of SEQ ID NOS: 3730-3737.

233. The method of claim 220, wherein the at least one guide RNA targets a TNFRSF11A gene.

234. The method of claim 233, wherein the at least one guide RNA comprises a crRNA sequence selected from the group consisting of SEQ ID NOS: 3323-3331.

235. The method of claim 233, wherein the at least one guide RNA comprises a crRNA sequence selected from the group consisting of SEQ ID NOS: 3749-3756.

236. The method of claim 199, wherein the transmembrane receptor is a transforming growth factor beta receptor.

237. The method of claim 236, wherein the at least one guide RNA targets a TGFBR1 gene.

238. The method of claim 237, wherein the at least one guide RNA comprises a crRNA sequence selected from the group consisting of SEQ ID NOS: 1535-1544.

239. The method of claim 237, wherein the at least one guide RNA comprises a crRNA sequence of 3792.

240. The method of claim 236, wherein the at least one guide RNA targets a TGFBR2 gene.

241. The method of claim 240, wherein the at least one guide RNA comprises a crRNA sequence selected from the group consisting of SEQ ID NOS: 1725-1734.

242. The method of claim 240, wherein the at least one guide RNA comprises a crRNA sequence selected from the group consisting of SEQ ID NOS: 3820-3822.

243. The method of any one of claims 114-242, wherein the disorder is a musculoskeletal disorder.

244. The method of claim 243, wherein the musculoskeletal disorder is selected from the group consisting of Rheumatoid Arthritis, Gout, Osteoarthritis, Osteoporosis, Intervertebral disc disease (IVDD), Psoriatic arthritis (PsA), Arthritis, Polymyositis, Proliferative synovitis, Malignant bone neoplasm, Sarcoid Myopathy, Cortex Bone Disorders, Idiopathic Scoliosis, Tendinopathy, Myofibrillar Myopathy, Enthesis-Related Arthritis, Ankylosing spondylitis, Degenerative, polyarthritis, Arthropathy, Osteitis Deformans, Prolapsed Lumbar Disc, Polymyositis Ossificans, Idiopathic Polymyositis, Luft Disease, Adult-onset Still's Disease, Osteoarthrosis Deformans, and Bachet's Disease.

245. The method of claim 243, wherein the musculoskeletal disorder is selected from the group consisting of Rheumatoid arthritis, Idiopathic osteoporosis, Post-menopausal osteoporosis, Paget's disease, Osteoarthritis, Juvenile idiopathic arthritis (JIA), Still's disease, Ankylosing spondylitis, Polymyalgia rheumatica, Arthritis, Secondary malignant neoplasm of bone, Type II, Mucolipidosis, Sjogren's Syndrome, Psoriatic arthritis, Rheumatism, Castleman's disease, Degenerative Polyarthritis, Arthropathy, Bone neoplasm, Osteoporosis, Massive osteolysis, Bone fracture healing, Systemic sclerosis, Systemic Juvenile Idiopathic, Arthritis, and Synovitis.

246. The method of claim 243, wherein the musculoskeletal disorder is selected from the group consisting of Arthritis, Infectious Intermittent joint effusion, Ankylosing spondylitis, Arthritis, Osteoarthritis, Spondylarthrites, Plantar fasciitis, Degenerative polyarthritis, Hemophilic arthropathy, Inflammatory myopathy with abundant macrophages, Polymyositis, Tendinosis, Malignant Bone Neoplasm, Osteoporosis, Psoriatic arthritis, Rheumatism, Rheumatoid arthritis, Adult Still's disease, Juvenile arthritis, Early rheumatoid arthritis, Palindromic rheumatism, Gout, Infectious Arthritis, Myotonic dystrophy, Dermatomyositis, Hemophilic arthropathy, Osteopenia, Sjogren's syndrome, Juvenile Idiopathic Arthritis, Myasthenia gravis, Osteolysis, Inflammation, Degenerative polyarthritis, Osteitis deformans, Pigmented villonodular synovitis, or Hyperphosphatasemia with bone disease.

247. The method of claim 243, wherein the musculoskeletal disorder is selected from the group consisting of Loeys-Dietz Syndrome, Osteoarthrosis, Marfan Syndrome, Aortic aneurysm (familial thoracic 3), and a Craniofacial abnormality.

248. The method of any one of claims 114-242, wherein the disorder is a neoplasia.

249. The method of claim 248, wherein the neoplasia is selected from the group consisting of Osteosarcoma, Colon Cancer, Metastasis (General), Lung Cancer, Multiple Myeloma. Breast Cancer, Solid Tumors, Lymphoma, Pancreatic Cancer, Stomach Cancer, Epithelial Ovarian Cancer, Mammary Neoplasms, Oropharyngeal Carcinoma, Renal Cell Carcinoma, Chondrosarcoma, Esophageal Neoplasms, B-Cell Lymphoma, Cutaneous Lymphoma T-Cell, Leukemia, Thyroid Carcinoma, Skin carcinogenesis, Cholectoral Cancer, Glioma, Liver Cancer, Melanoma, Neuroblastoma, Polycystic Ovary Syndrome, Glioblastoma, Prostate Cancer, Cervical Cancer, Ovarian Cancer, Bladder Cancer, Squamous cell carcinoma, Kaposi Sarcoma, Oral Cavity Cancer, Leiomyosarcoma, Malignant Peripheral Nerve Sheath Tumor, and Ewing's Sarcoma.

250. The method of claim 248, wherein the neoplasia is selected from the group consisting of Multiple myeloma, Lung Cancer, Stomach Cancer, Breast Cancer, Kidney Cancer, Neoplasm Metastasis, Colorectal Neoplasms, Ovarian cancer, Osteosarcoma, Cholangiocarcinoma, Leukemia, Prostate cancer, Plasmacytoma, Pancreatic cancer, Cervical cancer, Lymphoma, Liver neoplasms, Neuroblastoma, Melanoma, Mastocytosis, Endometrial cancer, Bladder Cancer, Squamous cell carcinoma, Gallbladder carcinoma, Adenocarcinoma, Thyroid Cancer, prostate neoplasms, stomach cancer, liver carcinoma, pituitary neoplasms, hepatoblastoma, multiple myeloma, hepatocellular adenoma, breast carcinoma, carcinogenesis, leukemia, colon carcinoma, melanoma, metastasis (general), lung cancer, pancreatic cancer, Kaposi sarcoma, medulloblastoma, carcinoma of bladder, squamous cell carcinoma, mast cell neoplasm, glioma, mastocytoma, brain neoplasms, ovarian neoplasms, bone neoplasm, rhabdomyosarcoma, solid tumors, metastatic kidney cancer, and metastatic renal cell carcinoma

251. The method of claim 248, wherein the neoplasia is selected from the group consisting of a Neoplasm, Glioblastoma, Astrocytoma, Adenocarcinoma, Osteosarcoma, Squamous cell carcinoma, Metastasis, Ameloblastoma, Cholangiocarcinoma, Choriocarcinoma, Ovarian cancer, Prostate cancer, Lung Cancer, Larynx Cancer, Breast Cancer, Lip and Oral Cavity Carcinoma, Squamous intraepithelial lesion, Neuroblastoma, Non-Hodgkin lymphomas, Malignant Neoplasms, Skin Neoplasms, Neuroblastoma, Neoplasm Metastasis, Colorectal Cancer, Fibrosarcoma, Myeloid Leukemia, Myelofibrosis, Hodgkin Lymphomas, Non-Small Cell Lung Carcinoma, Cervical Cancer, Liver cancer, Extrapulmonary small cell carcinoma, Basal cell carcinoma, Kidney cancer, Pancreatic carcinoma, Mesothelioma, Gastric cancer, Polycystic Ovary Syndrome, Chordoma, Cholangiocarcinoma, Malignant ascites, Nasopharyngeal carcinoma, Head and Neck Carcinoma, Cancer metastasis, Prostate carcinoma, Fibrosarcoma, Liver carcinoma, Carcinoma of bladder, T-Cell Lymphoblastic Leukemia, Bladder Neoplasm, melanoma, Leukemia, acute myeloid leukemia, Hepatocellular carcinoma, Colorectal cancer, a Lymphoma, Mycosis fungoides, and Sézary syndrome.

252. The method of claim 248, wherein the neoplasia is selected from the group consisting of Pancreatic cancer, Multiple self-healing squamous epithelioma (Ferguson-Smith disease), Pancreatic cancer, Gastrointestinal Stromal Tumors (GIST), Hereditary Nonpolyposis Colorectal Cancer (Lynch Syndrome), Metastasis, Colorectal Carcinoma, Bone neoplasms, Anaplastic carcinoma, Spindle-Cell carcinoma, Malignant Bone Neoplasm, Lung neoplasms, and Malignant brain neoplasm.

253. The method of any one of claims 114-242, wherein the disorder is a neurological disorder.

254. The method of claim 253, wherein the neurological disorder is selected from the group consisting of Acute Thrombotic Stroke, Epilepsy, Multiple Sclerosis, and Alzheimer's Disease.

255. The method of any one of claims 114-242, wherein the disorder is a cardiac disorder.

256. The method of claim 255, wherein the cardiac disorder is selected from the group consisting of Acute Myocardial Infarction, refractory heart failure, arrhythmias, pericarditis, myocarditis, sepsis-induced cardiomyopathy, Acute Decompensated Heart Failure, Refractory Idiopathic Pericarditis, Atherosclerosis, coronary artery disease, myocardial infarction, and cardiac remodeling.

257. The method of claim 255, wherein the cardiac disorder is atherosclerosis or abdominal aortic aneurism.

258. The method of any one of claims 114-242, wherein the disorder is an inflammatory disorder.

259. The method of claim 258, wherein the inflammatory disorder is selected from the group consisting of Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), and Fibrosis.

260. The method of claim 258, wherein the inflammatory disorder is selected from the group consisting of a cytokine storm, acute local inflammation, inflammatory bowel disease, Crohn's disease, sepsis, Experimental sepsis, and Castleman's disease

261. The method of any one of claims 114-242, wherein the disorder is a digestive disorder.

262. The method of claim 261, wherein the digestive disorder is selected from the group consisting of Inflammatory Bowel Disease (IBD), Gastroesophageal reflux disease (GERD), and Non-HP-associated Peptic Ulcer Disease.

263. The method of any one of claims 114-242, wherein the disorder is a respiratory disorder.

264. The method of claim 263, wherein the respiratory disorder is Asthma or Pulmonary Fibrosis.

265. The method of any one of claims 114-242, wherein the disorder is a renal, metabolic, or ophthalmic disorder.

266. The method of claim 265, wherein the renal, metabolic, or ophthalmic disorder is selected from the group consisting of Chronic Kidney Disease, Type 2 Diabetes, an Eye Disease, Uveitis, Scleritis, Sjogren asthenia, and dry eye.

267. The method of any one of claims 114-242, wherein the disorder is an autoimmune disorder.

268. The method of claim 267, wherein the autoimmune disorder is Systemic Lupus Erythematosus.

269. The method of any one of claims 114-242, wherein the disorder is an autoimmune or inflammatory disorder.

270. The method of claim 269, wherein the autoimmune or inflammatory disorder is selected from the group consisting of an Allergy, Asthma, Multiple sclerosis, Psoriasis, Inflammatory bowel disease, Autoimmune experimental uveitis, Colitis, Hypersensitivity reaction disease, Diabetes, Crohn's disease, Systemic lupus erythematosus, Pulmonary sarcoidosis, Leishmaniasis, Experimental autoimmune encephalomyelitis, HTLV-1-associated myelopathy, Tropical spastic paraparesis, Scleroderma, an Idiopathic inflammatory myopathy, polymyositis, and dermatomyositis.

271. The method of any one of claims 114-270, wherein the RNA-guided nuclease or a nucleic acid encoding an RNA-guided nuclease is the RNA-guided nuclease.

272. The method of any one of claims 114-270, wherein the RNA-guided nuclease or a nucleic acid encoding an RNA-guided nuclease is DNA encoding the RNA-guided nuclease.

273. The method of any one of claims 114-270, wherein the RNA-guided nuclease or a nucleic acid encoding an RNA-guided nuclease is mRNA encoding the RNA-guided nuclease.

274. The method of any one of claims 114-273, wherein the RNA-guided nuclease is a Cas protein.

275. The method of claim 274, wherein the Cas protein is a Cas9 protein.

276. The method of claim 274, wherein the Cas9 protein is an S. pyogenes Cas9 polypeptide.

277. The method of claim 274, wherein the Cas9 protein is selected from the group consisting of esCas9, hfCas9, peCas9, and ARCas9.

278. The method of any one of claims 114-277, wherein the at least one guide RNA or a nucleic acid encoding at least one guide RNA is the at least one guide RNA.

279. The method of any one of claims 114-277, wherein the at least one guide RNA or a nucleic acid encoding at least one guide RNA is DNA encoding the at least one guide RNA.

280. The method of any one of claims 114-277, comprising a nucleic acid encoding both the RNA-guided nuclease and the at least one guide RNA.

281. The method of any one of claims 114-280, wherein the at least one guide RNA is a single guide RNA (sgRNA).

282. The method of any one of claims 114-281, wherein the at least one guide RNA targets a human gene.

283. The method of any one of claims 114-281, wherein the at least one guide RNA targets a canine gene.

284. The method of any one of claims 114-281, wherein the at least one guide RNA targets an equine gene.

285. The method of any one of claims 114-281, wherein the at least one guide RNA targets a feline gene.

286. The method of any one of claims 114-281, wherein the at least one guide RNA targets a mammalian gene.

287. The method of any one of claims 114-286, wherein the composition comprises one or more viral vectors collectively comprising the (i) RNA-guided nuclease or a nucleic acid encoding an RNA-guided nuclease, and (ii) at least one guide RNA or a nucleic acid encoding at least one guide RNA targeting a gene encoding the transmembrane receptor.

288. The method of claim 287, wherein the one of more viral vectors comprise a recombinant virus selected from a retrovirus, an adenovirus, an adeno-associated virus, a lentivirus, and a herpes simplex virus-1.

289. The method of claim 287, wherein the one of more viral vectors comprise a recombinant adeno-associated virus (AAV).

290. The method of claim 289, wherein the recombinant AAV is of serotype 5 (AAV5).

291. The method of claim 289, wherein the recombinant AAV is of serotype 6 (AAV6).

292. The method of any one of claims 114-286, wherein the composition comprises one or more lipid nanoparticles (LNP) collectively comprising the (i) RNA-guided nuclease or a nucleic acid encoding an RNA-guided nuclease, and (ii) at least one guide RNA or a nucleic acid encoding at least one guide RNA targeting a gene encoding the transmembrane receptor.

293. The method of claim 292, wherein the one or more LNP comprises:
a first plurality of LNP encapsulating the RNA-guided nuclease or a nucleic acid encoding an RNA-guided nuclease; and
a second plurality of LNP encapsulating the at least one guide RNA or a nucleic acid encoding at least one guide RNA.

294. The method of claim 292, wherein the one or more LNP comprises a plurality of LNP encapsulating both the (i) RNA-guided nuclease or a nucleic acid encoding an RNA-guided nuclease, and (ii) at least one guide RNA or a nucleic acid encoding at least one guide RNA targeting a gene encoding the transmembrane receptor.

295. The method of any one of claims 292-294, wherein the one or more LNP comprises a component selected from the group consisting of 3-(didodecylamino)-N1,N1,4-tridodecyl-1-piperazineethanamine (KL10), N1-[2-(didodecylamino)ethyl]-N1,N4,N4-tridodecyl-1,4-piperazinediethanamine (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl 4-(dimethylamino) butanoate (DLin-MC3-DMA), 2,2-dilinoleyl-4-(2-dimethylaminoethyl)-[1,3]-dioxolane (DLin-KC2-DMA), 1,2-dioleyloxy-N,N-dimethylaminopropane (DODMA), 2-({8-[(3.beta.)-cholest-5-en-3-yloxy]octyl}oxy)-N,N-dimethyl-3-[(9Z,12Z)--octadeca-9,12-dien-1-yloxy]propan-1-amine (Octyl-CLinDMA), (2R)-2-({8-[(3.beta.)-cholest-5-en-3-yloxy]octyl}oxy)-N,N-dimethyl-3-[(9Z-,12Z)-octadeca-9,12-dien-1-yloxy]propan-1-amine (Octyl-CLinDMA (2R)), (2S)-2-({8-[(3.beta.)-cholest-5-en-3-yloxy]octyl}oxy)-N,N-dimethyl-3-[(9Z-,12Z)-octadeca-9,12-dien-1-yloxy]propan-1-amine (Octyl-CLinDMA (2S)), a lipid including a cyclic amine group, and a mixture thereof.

296. The method of any one of claims 292-295, wherein the LNP comprises a component selected from the group consisting of 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanolamine (DOPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin (SM), and a mixture thereof.

297. The method of any one of claims 292-296, wherein the LNP comprises a component selected from the group consisting of PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG-DLPE, PEG-DMPE, PEG-DPPC, PEG-DMA, a PEG-DSPE lipid, and a mixture thereof.

298. The method of any one of claims 96-100, wherein the LNP comprises a component selected from the group consisting of a cholesterol, fecosterol, stigmasterol, stigmastanol, sitosterol, β-sitosterol, lupeol, betulin, ursolic acid, oleanolic acid, campesterol, fucosterol, brassicasterol, ergosterol, 9,11-dehydroergosterol, tomatidine, tomatine, α-tocopherol, and a mixture thereof.

299. The method of any one of claims 114-286, wherein the composition comprises one or more liposomes collectively comprising the (i) RNA-guided nuclease or a nucleic acid encoding an RNA-guided nuclease, and (ii) at least one guide RNA or a nucleic acid encoding at least one guide RNA targeting a gene encoding the transmembrane receptor.

300. The method of claim 299, wherein the one or more liposomes comprises:
a first plurality of liposomes encapsulating the RNA-guided nuclease or a nucleic acid encoding an RNA-guided nuclease; and
a second plurality of liposomes encapsulating the at least one guide RNA or a nucleic acid encoding at least one guide RNA.

301. The method of claim 299, wherein the one or more liposomes comprises a plurality of liposomes encapsulating both the (i) RNA-guided nuclease or a nucleic acid encoding an RNA-guided nuclease, and (ii) at least one guide RNA or a nucleic acid encoding at least one guide RNA targeting a gene encoding the transmembrane receptor.

302. The method of any one of claims 114-286, wherein the composition comprises one or more virus-like particles collectively comprising the (i) RNA-guided nuclease or a nucleic acid encoding an RNA-guided nuclease, and (ii) at least one guide RNA or a nucleic acid encoding at least one guide RNA targeting a gene encoding the transmembrane receptor.

303. The method of claim 302, wherein the one or more virus-like particles comprises:
a first plurality of virus-like particles encapsulating the RNA-guided nuclease or a nucleic acid encoding an RNA-guided nuclease; and
a second plurality of virus-like particles encapsulating the at least one guide RNA or a nucleic acid encoding at least one guide RNA.

304. The method of claim 302, wherein the one or more virus-like particles comprises a plurality of virus-like particles encapsulating both the (i) RNA-guided nuclease or a nucleic acid encoding an RNA-guided nuclease, and (ii) at least one guide RNA or a nucleic acid encoding at least one guide RNA targeting a gene encoding the transmembrane receptor.

305. The method of any one of claims 114-304, wherein the composition is formulated for parenteral administration.

306. The method of any one of claims 114-304, wherein the composition is formulated for intra-articular injection within a joint of the subject.

307. The method of any one of claims 114-304, wherein the composition is formulated for intradiscal injection.

308. The method of any one of claims 114-304, wherein the composition is formulated for peridiscal injection.

309. The method of any one of claims 114-304, wherein the composition is formulated for intravertebral injection.

310. The method of any one of claims 114-304, wherein the administering comprises parenteral administration.

311. The method of any one of claims 114-304, wherein the administering comprises intra-articular injection within a joint of the subject.

312. The method of any one of claims 114-304, wherein the administering comprises intradiscal injection.

313. The method of any one of claims 114-304, wherein the administering comprises peridiscal injection.

314. The method of any one of claims 114-304, wherein the administering comprises intravertebral injection.

315. The method of any one of claims 114-304, wherein the administering comprises intra-articular injection of the pharmaceutical composition into the joint of the subject.

316. The method of any one of claims 114-315, wherein the pharmaceutical composition is administered during surgery.

317. The method of any one of claims 114-316, wherein the pharmaceutical composition is administered after surgery.

318. The method of any one of claims 114-317, wherein the pharmaceutical composition is a controlled release pharmaceutical composition.
